(19) United States
(12) Patent Application Publication
(10) Pub. No.: US 2010/0132071 A1
(43) Pub. Date: May 27, 2010

(54) Plants Having Enhanced Yield-Related Traits And A Method For Making The Same

(75) Inventors: Yves Hatzfeld, Lille (FR); Ana Isabel Sanz Molinero, Gentbrugge (BE); Amber Shirley, Durham, NC (US); Lalitree Darnielle, Durham, NC (US); Valerie Frankard, Waterloo (BE); Steven Vandenabeele, Oudenaarde (BE); Bryan McKersie, Raleigh, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/598,055

(22) PCT Filed: May 2, 2008
(86) PCT No.: PCT/US08/05732

§ 371 Date: Oct. 29, 2009

Related U.S. Application Data
(60) Provisional application No. 60/916,575, filed on May 8, 2007.
(60) Provisional application No. 60/942,214, filed on Jun. 6, 2007.
(60) Provisional application No. 60/937,989, filed on Jun. 29, 2007.
(60) Provisional application No. 60/948,036, filed on Jul. 5, 2007.

(30) Foreign Application Priority Data
May 3, 2007 (EP) 07107448.8
May 29, 2007 (EP) 07109052.6
May 29, 2007 (EP) 07109068.2
Jun. 11, 2007 (EP) 07109961.8
Jun. 19, 2007 (EP) 07110548.0
Jun. 19, 2007 (EP) 07110557.1

(51) Int. Cl. C12N 15/82; C12N 15/29; C07K 14/415; A01H 5/00; A01H 5/10

(52) U.S. Cl. 800/290; 800/278; 536/23.6; 530/370; 800/298; 435/320.1; 800/320.2; 800/320.1; 800/320; 800/320.3

(57) ABSTRACT

The present invention relates generally to the field of molecular biology and concerns a method for enhancing yield-related traits and/or improving various plant growth characteristics by modulating expression in a plant of a nucleic acid encoding a GRP (Growth Regulating Protein). The GRP is selected from a LOB-domain comprising protein (LOB: Lateral Organ Boundaries), herein abbreviated as LBD polypeptide, a JMJC (JUMONJI-C) polypeptide, a CKI (Casein Kinase I) polypeptide, a bHLH11-like (basic Helix-Loop-Helix 11) protein, a plant homeodomain finger-homeodomain (PHDf-HD) polypeptide, an ASR (abscisic acid-, stress-, and ripening-induced) polypeptide and/or a Squamosa promoter binding protein-like 1 1 (SPL11) transcription factor polypeptide. The present invention also concerns plants having modulated expression of a nucleic acid encoding a GRP, which plants have improved growth characteristics relative to corresponding wild type plants or other control plants. The invention also provides novel GRP nucleic acids and GRP polypeptides as well as constructs useful in the methods of the invention.

The present invention relates generally to the field of molecular biology and concerns a method for enhancing yield-related traits and/or improving various plant growth characteristics by modulating expression in a plant of a nucleic acid encoding a GRP (Growth Regulating Protein). The GRP is selected from a LOB-domain comprising protein (LOB: Lateral Organ Boundaries), herein abbreviated as LBD polypeptide, a JMJC (JUMONJI-C) polypeptide, a CKI (Casein Kinase I) polypeptide, a bHLH11-like (basic Helix-Loop-Helix 11) protein, a plant homeodomain finger-homeodomain (PHDf-HD) polypeptide, an ASR (abscisic acid-, stress-, and ripening-induced) polypeptide and/or a Squamosa promoter binding protein-like 11 (SPL11) transcription factor polypeptide. The present invention also concerns plants having modulated expression of a nucleic acid encoding a GRP, which plants have improved growth characteristics relative to corresponding wild type plants or other control plants. The invention also provides novel GRP nucleic acids and GRP polypeptides as well as constructs useful in the methods of the invention.

The ever-increasing world population and the dwindling supply of arable land available for agriculture fuels research towards increasing the efficiency of agriculture; Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigor has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

Surprisingly, it has now been found that modulating expression of a nucleic acid encoding a GRP polypeptide selected from a LOB-domain comprising protein (LOB: Lateral Organ Boundaries), herein abbreviated as LBD polypeptide, a JMJC (JUMONJI-C) polypeptide, a casein kinase polypeptide, a plant homeodomain finger-homeodomain (PHDf-HD) polypeptide, a bHLH11-like (basic Helix-Loop-Helix 11) protein, an ASR (abscisic acid-, stress-, and ripening-induced) polypeptide and/or a Squamosa promoter binding protein-like 11 (SPL11) transcription factor polypeptide gives plants having enhanced yield-related traits and/or improved various plant growth characteristics relative to control plants.

According one embodiment, there is provided a method for improving or enhancing yield related traits and/or improving various plant growth characteristics of a plant relative to control plants, comprising modulating expression of a nucleic acid encoding a GRP polypeptide selected from a LOB-domain comprising protein (LOB: Lateral Organ Boundaries), herein abbreviated as LBD polypeptide, a JMJC (JUMONJI-C) polypeptide, a casein kinase polypeptide, a plant homeodomain finger-homeodomain (PHDf-HD) polypeptide, a bHLH11-like (basic Helix-Loop-Helix 11) protein, an ASR (abscisic acid-, stress-, and ripening-induced) polypeptide and/or a Squamosa promoter binding protein-like 11 (SPL11) transcription factor polypeptide in a plant.

### BACKGROUND

### 1. LOB-Domain Comprising Protein (LOB: Lateral Organ Boundaries)

LBD proteins (or LOB domain proteins) all share a conserved domain in the N-terminal region, known as the LOB domain. LOB domain proteins (Shuai et al., Plant Physiol. 129, 747-761, 2002) are found in various plant species and constitute a large gene family: Arabidopsis is reported to possess more than 40 genes encoding LOB domain proteins, at least 35 genes are found in rice and at least 15 genes in maize. LBD polypeptides may be regulators of transcription factors (among which KNOX transcription factors) and are postulated to play a role in tassel and ear branching in maize (Bortiri et al., Plant Cell 18, 574-587, 2006), formation of adventitious roots (Liu et al., Plant J. 43, 47-56, 2005; Inukai et al., Plant Cell 17, 1387-1396, 2005), proliferation of the female gametophyte (Evans et al., Plant Cell 19, 46-62, 2007), proximal-distal patterning in petals (Chalfun-Junior et al, Plant Mol. Biol. 57, 559-575, 2005); leaf morphology and venation (Iwakawa et al., Plant Cell Physiol. 43, 467-478, 2002). Yang et al. (Molecular Phylogenetics and Evolution 39, 248-262, 2006) discriminated three classes of LBD proteins in rice, based on the classification of Iwakawa et al. (2005) and Shuai et al. (2002). Modulation of class I LOB gene expression (up or downregulation of expression) often results in pleiotropic effects, leading to abnormal plant shape and infertility. For example, US20060218674 discloses a method for increasing the size of the endosperm in a plant seed by expressing a class I LOB polypeptide, however the size of the embryo decreased proportionally.

Surprisingly, it has now been found that modulating expression of a nucleic acid encoding a LBD polypeptide gives plants having enhanced yield-related traits, in particular increased biomass and seed yield, relative to control plants.

According one embodiment, there is provided a method for improving yield related traits of a plant relative to control plants, comprising modulating expression of a nucleic acid encoding a LBD polypeptide in a plant. The improved yield related traits comprised increased biomass and increased seed yield.

### II. JMJC (JUMONJI-C) Polypeptide

The first reported JUMONJI (which means cruciform in Japanese) gene was identified by gene trapping in mice where it plays an essential role in the development of multiple tissues. To date JUMONJI polypeptides constitute a distinct class of proteins found in prokaryotes as well as in eukaryotes including bacteria, fungi and plants.

Most members of the JUMONJI polypeptide family are characterized by the presence of a JmjC or JUMONJI-C domain. It is hypothesized that during evolution ancient proteins comprising only on JmjC domain acquired additional domains broadening the spectrum of JMJC polypeptides found in nature. Of particular interest are those JMJC polypeptides that have acquired conserved domains involved in DNA, RNA, and protein binding such as zinc fingers, FY-rich, RING finger protein and F-box domains, suggesting that polypeptides of the JUMONJI family may regulate transcription, chromatin function and/or protein turnover. Accordingly many JUMONJI proteins in animals have been reported to affect development by controlling gene expression and chromatin activity. For example a mouse JUMONJI gene acts to repress cyclin D1 in embryos and this activity is required for normal cardiogenesis (Toyoda et al. 2003 Dev Cell. 5(1):85-97.).

Much progress has been made on the understanding of the mode of action of jmjC domain containing proteins. Crucial to their biological function may be some of the recently revealed enzymatic activities in JMJC polypeptides, for example JHDM1, a JMJC polypeptide of human origin (Tsukada, Nature. 2006; 439(7078):811-6) has histone dimethylase activity, and asparaginyl hydroxylase activity has been reported in FIH (Factor Inhibiting HIF-1alpha), a transcription factor involved in cellular response to hypoxia, (Linke et al. (2004) J. Biol. Chem., Vol. 279, 14391-14397).

JmjC domains typically have structures resembling those found in some metalloenzymes. Recent structural analysis of the JmjC domain present in the JUMONJI protein FIH revealed the amino acid residues involved in binding to the cofactors 2-oxoglutarate and iron Fe (II) (Dann et al; Proc Natl Acad Sci USA. 2002; 99(24): 15351-15356). FIH has the HXD/E iron-binding motif characteristic of most of the 2-oxoglutarate oxygenases. In addition and consistent with the hydrolase activity, FIH secondary structure is composed of a beta-strand jellyroll core that surrounds the Fe(II)-binding site. These features are conserved amongst JmjC domain containing proteins (Trewick et al. EMBO Rep. 2005 (4):315-20).

In plants, two JMJC polypeptides are reportedly involved in the control of flowering time. They both act as repressors of flowering pathways in Arabidopsis thaliana (Noh et al. Plant Cell. 2004. 16(10):2601-13). Enzymatic activity for the plant proteins has not yet been experimentally determined.

Surprisingly, it has now been found that modulating expression in a plant of a nucleic acid encoding a JUMONJI-C or JMJC polypeptide gives plants having enhanced yield-related traits, in particular increased yield relative to control plants.

According to one embodiment, there is provided a method for improving yield-related traits of a plant relative to control plants, comprising modulating expression of a nucleic acid encoding a JMJC polypeptide in a plant. The improved yield related traits comprise one or more of increased total seed weight per plant, thousand-kernel (1000-seed) weight, seed filling rate, harvest index, early vigour, and root/shoot index, both under optimal growth conditions and suboptimal, mild drought conditions.

### III. Casein Kinase I

Protein kinases represent a superfamily, and the members of this superfamily catalyze the reversible transfer of a phosphate group of ATP to serine, threonine, and tyrosine amino acid side chains on target polypeptides. In particular, the Casein kinase 1 (CKI) family (EC 2.7.11.1) of protein kinases function as regulators of signal transduction pathways in most eukaryotic organisms. In yeast CKI is involved in the regulation of DNA repair and cell cycle progression (Hoekstra M F et al., Science 253: Brockman J L et al., Proc. Natl Acad. Sci. USA 89: 9454-9458, 1992; Dhillon N and Hoekstra M F, EMBO J. 13: 2777-2788, 1994). Casein kinase I proteins are monomeric serine/threonine type protein kinases that contain a highly conserved central kinase domain. Members of this family have divergent N-terminal and C-terminal extensions. The N-terminal region is responsible for substrate recognition and the C-terminal extension is important for the interaction of the kinase with substrates. The C-terminal extension also is thought to be important for mediating regulation through auto-phosphorylation (Gross and Anderson, 1 998 Cell Signal 10:699-71 1; Craves and Roach, 1 995, J Biol Chem 270:21689-21694).

In plants several CKI protein family members have been cloned and characterised biochemically (Klimczak and Cashmore A R, 1993. Biochem. J. 293: 283-288; Liu et al. 2003, Plant Journal. 36, 189-202; Lee et al. 2005 Plant Cell. 17(10): 2817-2831. The Arabidopsis genome was found to contain at least 14 Casein Kinase I-like (CKL) genes. Within the conserved kinase domains, the polypeptides shared 89% sequence similarity at the amino acid level. The 14 Arabidopsis CKL isoforms have been further classified in three groups based on the subcellular localization. Group 1 localized predominantly at the cell periphery; group 2 in the nucleus group 3 in the cytoplasm. A Nicotiana tabacum CKI has been localized to plasmodesmata and propose to play a role in cell to cell communication (Lee et al. 2005). Other proposed roles for plant CKI is signal truduction in response to environmental stimuli, root development and plant hormone sensitivity (Liu et al.).

Surprisingly, it has now been found that modulating expression of a nucleic acid encoding a CKI polypeptide gives plants having enhanced yield-related traits relative to control plants.

According to one embodiment, there is provided a method for improving yield related traits of a plant relative to control plants, comprising modulating expression of a nucleic acid encoding a CKI polypeptide in a plant. The improved yield related traits comprised one or more of increased biomass, increased emergency vigour, and increased seed yield.

### IV. Plant Homeodomain Finger-Homeodomain (PHDf-HD) Polypeptide

Transcription factors are usually defined as proteins that show sequence-specific DNA binding affinity and that are capable of activating and/or repressing transcription. The Arabidopsis thaliana genome codes for at least 1533 transcriptional regulators, accounting for ˜5.9% of its estimated total number of genes (Riechmann et al. (2000) Science 290: 2105-2109). The Database of Rice Transcription Factors (DRIP) is a collection of known and predicted transcription factors of Oryza sativa L. ssp. indica and Oryza sativa L. ssp. japonica, and currently contains 2,025 putative transcription factors (TF) gene models in indica and 2,384 in japonica, distributed in 63 families (Gao et al. (2006) Bioinformatics 2006, 22(10):1286-7).

One of these families is the superfamily of homeodomain (HD) transcription factors involved in many aspects of developmental processes. HD transcription factors are characterized by the presence of a homeodomain (HD), which is a 60-amino acid DNA-binding domain (30). Arabidopsis thaliana and rice contain approximately 100 HD transcription factors, which can be further classified into subfamilies based on amino acid sequence identity (Richardt at al. (2007) Plant Phys 143(4): 1452-1466). Some of these subfamilies are characterized by the presence of additional conserved domains that facilitate DNA binding and/or protein-protein interactions.

One of these domains is the PHD finger, named plant homeodomain finger (PHDf) due to its association on a same polypeptide with a DNA-binding HD, that was originally identified by amino acid sequence identity between a maize homeobox transcription factor ZmHOX1a (Bellman & Werr (1992) EMBO J. 11:3367-3374) and its Arabidopsis relative ATHAT3.1 (Schindler et al. (1993) Plant J 4: 137-150). The PHDf is a Cys₄-His-Cys₃ zinc-finger-like motif capable of chelating two zinc ions. PHDfs are found in nuclear proteins and are thought to be involved in chromatin-mediated transcriptional regulation (Halbach et al., (2000) Nucleic Acid Res 28(18): 3542-3550).

Transcriptional factors combining a PHDf and a HD are therefore named PHDf-HDs (Halbach at al. (2000) supra). In plants, such PHDf-HDs are further characterized by the presence of a leucine zipper (ZIP) upstream of the PHDf. Both domains (the ZIP and the PHDf) together form a highly conserved 180 amino acid region called the ZIP/PHDf domain (Halbach at al. (2000) supra).

Transgenic tobacco plants strongly overexpressing either of two maize PHDf-HD polypeptides (ZmHOX1a or ZmHOX1b) using a cauliflower mosaic virus 35S promoter combined with an omega enhancer, showed identical morphological changes: size reduction, adventitious root formation, and homeotic floral transformations (Uberlacker at al. (1996) Plant Cell 8: 349-362). Transgenic rice and tobacco plants strongly overexpressing Oryza sativa NAZI PHDF-HD polypeptide using cauliflower mosaic virus 35S promoter showed no abnormal growth or phenotypic change compared to wild types (Ito at al. (2004) Gene 331: 9-15).

In U.S. Pat. No. 7,196,245, an Arabidopsis thaliana PHDf-HD polypeptide (identified as G416) was transformed into Arabidopsis, and shown to promote early flowering in the transgenic plants compared to control plants, with no impact on seed yield.

Surprisingly, it has now been found that modulating, preferably increasing, expression of a nucleic acid sequence encoding a PHDf-HD polypeptide gives plants having enhanced yield-related traits, preferably enhanced seed yield-related traits, relative to control plants.

According one embodiment, there is provided a method for enhancing yield-related traits, preferably enhancing seed yield-related traits, in plants relative to control plants, comprising modulating, preferably increasing, expression of a nucleic acid sequence encoding a PHDf-HD polypeptide in a plant. The enhanced yield-related traits, preferably enhanced seed yield-related traits, comprise one or more of: increased number of primary panicles, increased total seed yield per plant, increased number of (filled) seeds, increased thousand kernel weight (TKW), increased harvest index.

V. bHLH11-Like (Basic Helix-Loop-Helix 11) Protein

Transcription factors are usually defined as proteins that show sequence-specific DNA binding and that are capable of activating and/or repressing transcription. The basic Helix-Loop-Helix transcription factor family is one of the largest families of transcription factors that have been characterised in Arabidopsis thaliana (Toledo-Ortiz et al., Plant Cell 15, 1749-1770, 2003; Bailey et al., Plant Cell 15, 2497-2501, 2003) and in rice (Li et at Plant Physiol. 141, 1167-1184, 2006). The distinguishing characteristic of the bHLH transcription factor family is the presence of a bipartite domain consisting of approximately 60 amino acids. This bipartite domain is comprised of a DNA-binding basic region, which binds to a consensus hexanucleotide E-box and two α-helices separated by a variable loop region, located C-terminally of the basic domain. The two α-helices promote dimerisation, allowing the formation of homo- and heterodimers between different family members. While the bHLH domain is evolutionarily conserved, there is little sequence similarity between clades beyond the domain. Li et al. (2006) classify the rice and Arabidopsis bHLH transcription factors into 22 subfamilies, based on the sequence of the bHLH domains.

Little is known about the function of bHLH11-like polypeptides in plants. So far, only one bHLH11-like polypeptide, OsPTF1 from rice, has been characterised. OsPTF1 is reported to be involved in tolerance to phosphate starvation (Yi et al., Plant Physiol. 138, 2087-2096). Rice plants overexpressing this gene under control of the 358 promoter did not show any different phenotype compared to control plants when grown under normal conditions, but under conditions of phosphate limitation, the plants had an improved phosphate uptake. Under phosphate limitation, the transgenic plants showed an increase in biomass, phosphate content, increased tillering and increased seed yield.

Surprisingly, it has now been found that modulating expression I a plant of a nucleic acid encoding a bHLH11-like polypeptide gives plants having enhanced yield-related traits, in particular increased yield relative to control plants. These effects were shown under growth conditions where phosphate was not limiting.

According one embodiment, there is provided a method for improving yield related traits of a plant relative to control plants, comprising modulating expression of a nucleic acid encoding a bHLH11-like polypeptide in a plant. The improved yield related traits comprised increased seed yield.

### VI. ASR (Abscisic Acid-, Stress-, and Ripening-Induced) Protein

ASR (abscisic acid-, stress-, and ripening-induced) polypeptides were first identified in tomato (Iusem et al 1993 Plant Physiol 102: 1353-1354) as small highly charged hydrophyllic proteins localized to the nuclei of the cells and bound to chromatin. The Asr gene family encoding ASR polypeptides is found widespread in higher plants and ASR homologs have cloned from a large number of dicot and monocot plants (Carrai et al. 2004 Trends Plant Sci 9: 57-59). Most Asr genes are up-regulated under different environmental stress conditions, during fruit ripening, and upon cellular treatment with the hormone ABA. ASR polypeptides show a high a degree of sequence conservation (Frankel et al. 2006. Gene Pages 74-83). All known Asr genes contain two highly conserved regions. The first region contains a stretch of His residues at the N-terminus, possessing sequence-specific Zn²⁺-dependent DNA binding activity (Kalifa et al., 2004a Biochem J 381: 373-378). The second region is a large part of the C-terminal sequence, often containing a nuclear localization signal NLS (Cakir et al., 2003 Plant Cell 15: 2165-2180). ASR1 protein of tomato is an intrinsically unstructured protein which upon binding of zinc ions, becomes ordered (folded) and forms dimers (Goidgur et al. Plant Physiol. 2007 February; 143(2):617-28)

A putative role of ASR polypeptides in regulation of gene transcription has been suggested. Reportedly, yeast-one-hybrid experiments revealed that a grape ASR binds to the promoter of a hexose transporter gene (VvHT1). Consistently, a role of Asr1 in the control of hexose uptake in heterotrophic organs such as potato tubers has been suggested (Frankel et al. Plant Mol. Biol. 2007 March; 63(5):719-30). Zinc-dependent DNA binding activity of a protein member of the ASR family has been reported (Kalifa et al. 2004 Biochem J. 2004 Jul. 15; 381(Pt 2):373-8). DNA- and zinc-binding domains of ASR1 protein have been mapped (Rom et al. 2006. Biochimie. 88(6):621-8; Goldgur et al. 2007. Plant Physiol. February; 143(2):617-28).

The use of ASR polypeptides to improve agronomic traits in plants has been disclosed, as methods to enhance tolerance of plant to particular abiotic stresses. For example, overexpression in Arabidopsis (Arabidopsis thaliana) of the ASR1 ortholog LLA23 gene from lily (Lilium longiflorum) increases the plant tolerance to drought and salinity (Yang et al. 2005. Plant Physiol 139: 836-846). Further, U.S. Pat. No. 7,154,025 discloses methods for increasing resistance to water deficit stress by increasing amount of ASR proteins in a plant.

Surprisingly, it has now been found that modulating expression in a plant of a nucleic acid encoding an ASR polypeptide gives plants having enhanced yield-related traits under non-stress growth conditions, relative to control plants. According to a first embodiment, the present invention provides a method for enhancing yield-related traits under non-stress growth conditions in plants relative to control plants, comprising modulating expression in a plant of a nucleic acid encoding an ASR polypeptide.

### VII. Squamosa Promoter Binding Protein-Like 11 (SPL11)

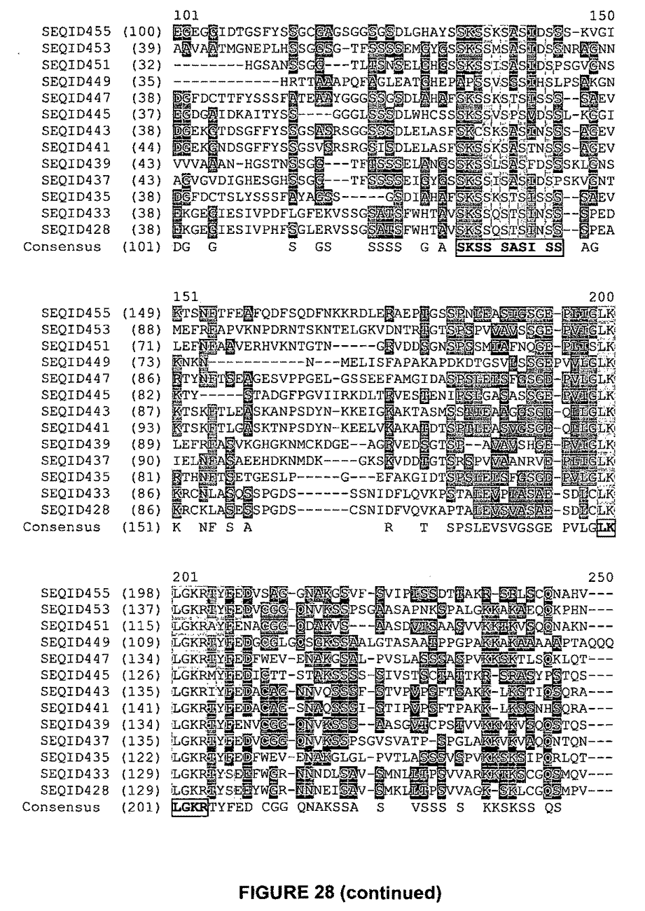

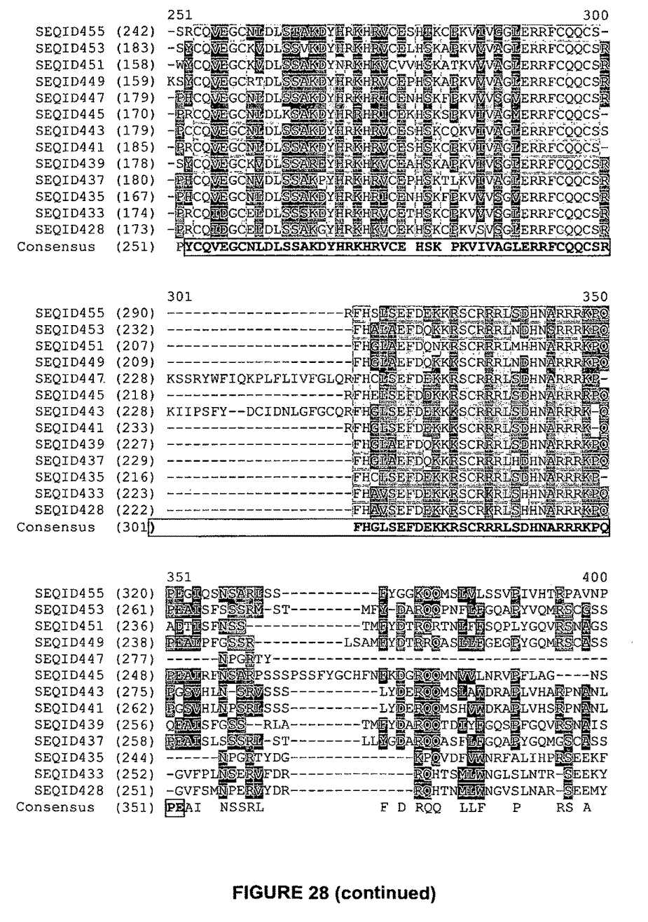

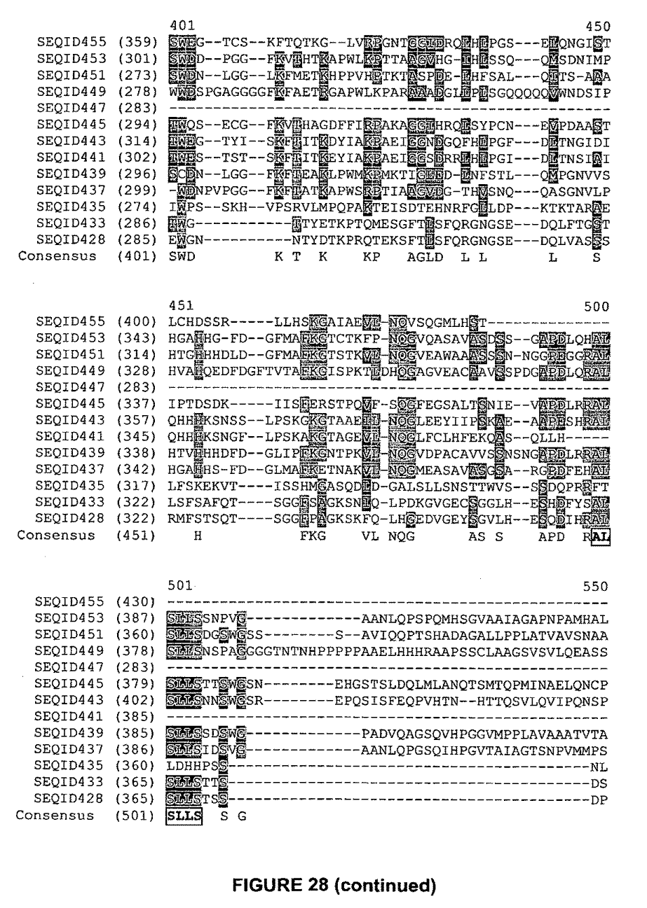

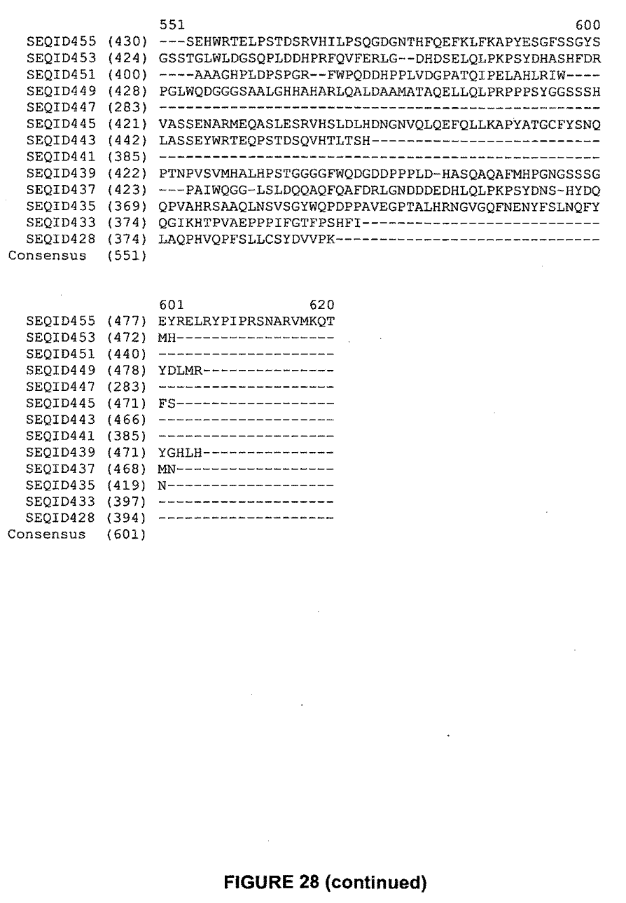

Transcription factor polypeptides are usually defined as proteins that show sequence-specific DNA binding affinity and that are capable of activating and/or repressing transcription. The Squamosa promoter binding protein-like (SPL) transcription factor polypeptides are structurally diverse proteins that share a highly conserved DNA binding domain (DBD) of about 80 amino acid residues in length (Klein et al. (1996) Mol Gen Genet. 259: 7-16; Cardon et al. (1999) Gene 237: 91-104). The SPL transcription factor DNA consensus sequence binding site in the promoter of target genes is 5′-TNCGTACAA-3′ where N represents any base. Within the SPL DBD are ten conserved cysteine (Cys) or histidine (His) residues (see FIG. 28) of which eight are zinc coordinating residues binding two zinc ions necessary for the formation of SPL specific zinc finger tertiary structure (Yamasaki et al. (2004) J Mol Biol 337: 49-63). A second conserved feature within the SPL DBD is a bipartite nuclear localisation signal. Outside of the DBD, a micro RNA (miRNA) target motif, specifically targeted by miR156 family of miRNAs is found in most of the nucleic acid sequences encoding SPL transcription factor polypeptides (either in the coding region, or the 3′ UTR) across the plant kingdom (Rhoades et al. (2002) Cell 110: 513-520). miRNAs control SPL gene expression post-transcriptionally by targeting SPL encoding mRNAs for degradation or by translational repression.

The Arabidopsis genome codes for at least 1533 transcriptional regulators, accounting for ˜5.9% of its estimated total number of genes (Riechmann et al. (2000) Science 290: 2105-2109). The authors report 16 SPL transcription factor polypeptides in Arabidopsis thaliana, with little sequence similarity among themselves (apart from the abovementioned features), the size of the deduced SPL polypeptide ranging from 131 to 927 amino acids. Nevertheless, pairs of SPL transcription factor polypeptides sharing higher sequence homology were detected within the SPL family of this plant (Cardon et al, (1999)).

The SPL transcription factor polypeptides (only found in plants) characterized to date have been shown to function in plant development, in particular in flower development. Transgenic plants overexpressing an SPL3 transcription factor polypeptide were reported to flower earlier (Cardon et al. (1997) Plant J 12: 367-377). In European patent application EP1033405, the nucleic acid and deduced polypeptide sequences of the SPL11 transcription factor polypeptide are disclosed.

Surprisingly, it has now been found that modulating expression I a plant of a nucleic acid encoding a SPL11 polypeptide gives plants having enhanced yield-related traits, in particular increased yield relative to control plants.

According to one embodiment, there is provided a method for enhancing yield related traits of a plant relative to control plants, comprising modulating expression of a nucleic acid encoding a SPL11 polypeptide in a plant. The enhanced yield related traits comprise one or more of increased emergence vigour (improved seedling early vigour), total seed yield (seed weight), seed fill rate (seed filling rate), number of filled seeds, number of flowers (seeds) per panicle, harvest index and thousand-kernel (1000-seed) weight, such increase occurring both under optimal and suboptimal growth conditions, preferably mild drought conditions.

According to one embodiment, there is provided novel SPL11 nucleic acids and SPL11 polypeptides as well as constructs comprising SPL11-encoding nucleic acids, useful in performing the methods of the invention

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequences)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thy | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tm=79.8+18.5(log₁₀[Na⁺]^{a})+0.58(% G/C^{b})+11.8(% G/C^{b})²−820/L^{c}

3) oligo-DNA or oligo-RNA^{d} hybrids:

For <20 nucleotides: Tₘ=2(lₙ)

For 20-35 nucleotides: Tₘ=22+1.46(lₙ)

^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; lₙ=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, |
| | 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco | U.S. Pat. No. 4,962,028 |
| small | |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 Jan; 27(2): 237-48 |
| Arabidopsis PHT1 | Kovama et al., 2005; Mudge et al. (2002, Plant J. 31: 341) |
| Medicago phosphate transporter | Xiao et al., 2006 |
| Arabidopsis Pyk10 | Nitz et al. (2001) Plant Sci 161(2): 337-346 |
| root-expressible genes | Tingey et al., EMBO J. 6: 1, 1987. |
| tobacco auxin-inducible gene | Van der Zaal et al., Plant Mol. Biol. 16, 983, 1991. |
| β-tubulin | Oppenheimer, et al., Gene 63: 87, 1988. |
| tobacco root-specific genes | Conkling, et al., Plant Physiol. 93: 1203, 1990. |
| B. napus G1-3b gene | U.S. Pat. No. 5,401,836 |
| SbPRP1 | Suzuki et al., Plant Mol. Biol. 21: 109-119, 1993. |
| LRX1 | Baumberger et al. 2001, Genes & Dev. 15: 1128 |
| BTG-26 Brassica napus | US 20050044585 |
| LeAMT1 (tomato) | Lauter et al. (1996, PNAS 3: 8139) |
| The LeNRT1-1 (tomato) | Lauter et al. (1996, PNAS 3: 8139) |
| class I patatin gene (potato) | Liu et al., Plant Mol. Biol. 153: 386-395, 1991. |
| KDC1 (Daucus carota) | Downey et al. (2000, J. Biol. Chem. 275: 39420) |
| TobRB7 gene | W Song (1997) PhD Thesis, North Carolina State |
| | University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. plumbaginifolia) | Quesada et al. (1997, Plant Mol. Biol. 34: 265) |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2d, 2e, 2f. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22. 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW glutenin-1 | Mol Gen Genet 216: 81-90, 1989; NAR 17: 461-2, 1989 |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98: 1253-62, 1999; Plant J 4: 343-55, 1993; |
| | Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, 1996 |
| rice α-globulin REB/OHP-1 | Nakase et al. Plant Mol. Biol. 33: 513-522, 1997 |
| rice ADP-glucose pyrophosphorylase | Trans Res 6: 157-68, 1997 |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW and | Colot et al. (1989) Mol Gen Genet 216: 81-90, |
| HMW glutenin-1 | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, | Cho et al. (1999) Theor Appl Genet 98: 1253-62; |
| hordein | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin | Wu et al, (1998) Plant Cell Physiol 39(8) 885-889 |
| NRP33 | |
| rice globulin Glb-1 | Wu et al. (1998) Plant Cell Physiol 39(8) 885-889 |
| rice globulin | Nakase et al. (1997) Plant Molec Biol 33: 513-522 |
| REB/OHP-1 | |
| rice ADP-glucose | Russell et al. (1997) Trans Res 6: 157-68 |
| pyrophosphorylase | |
| maize ESR gene | Opsahl-Ferstad et al. (1997) Plant J 12: 235-46 |
| family | |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, |
| | 93: 8117-8122, 1996 |
| KNOX | Postma-Haarsma et al, Plant Mol. Biol. |
| | 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase (Amy32b) | Lanahan et al, Plant Cell 4: 203-211, 1992; |
| | Skriver et al, Proc Natl Acad Sci USA 88: |
| | 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g.

TABLE 2g
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, from | Sato et al. (1996) |
| | embryo globular stage to | Proc. Natl. Acad. Sci. |
| | seedling stage | USA, 93: 8117-8122 |
| Rice | Meristem specific | BAD87835.1 |
| metallothionein | | |
| WAK1 & WAK 2 | Shoot and root apical | Wagner & Kohorn |
| | meristems, and in expanding | (2001) Plant Cell |
| | leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required, In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al, (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22287; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette for example the naturally occurring combination of the natural promoter of the nucleic acid sequences with the corresponding nucleic acid sequence encoding a polypeptide useful in the methods of the present invention, as defined above becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term ‘introduction’ or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Bid 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan at al. (Plant Mol Biol 22 (3): 491-506, 1993), Hiei at al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in; Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et at, (2000) Nat Biotechnol 18: 455-457; reviewed by Stemple (2004) Nat Rev Genet. 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; Iida and Terada (2004) Curr Opin Biotech 15(2): 132-8), and approaches exist that are generally applicable regardless of the target organism (Miller et al, Nature Biotechnol. 25, 778-785, 2007).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium app., Amaranthus spp., Ammophila arenaria, Ananas comosus, Annona app., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum app., Carex elata, Carica papaya, Carissa macrocarpa, Carya spp., Carthamus tinctorius, Castanea spp., Ceiba pentandra, Cichorium endivia, Cinnamomum spp., Citrullus ianatus, Citrus spp., Cocos spp., Coffea spp., Colocasia esculenta, Cola spp., Corchorus sp., Coriandrum sativum, Corylus spp., Crataegus spp., Crocus sativus, Cucurbita app., Cucumis app., Cynara spp., Daucus carols, Desmodium app., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Sofa hispida or Sofa max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma app., Malus spp., Malpighia emarginata, Mammea americana, Mangifera indica, Manihot spp., Manilkara zapota, Medicago sativa, Melilotus spp., Mentha spp., Miscanthus sinensis, Momordica app., Morus nigra, Musa spp., Nicotiana spp., Olea spp., Opuntia spp., Ornithopus spp., Oryza spp. (e.g. Oryza sativa, Oryza latifolia), Panicum millaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis app., Pinus app., Pistacia vera, Pisum spp., Poa spp., Populus spp., Prosopis app., Prunus spp., Psidium spp., Punica granatum, Pyrus communis, Quercus app., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solarium integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale timpaui, Triticum spp. (e.g. Triticum aestivum, Triticum durum, Triticum turgidum, Triticum hybemum, Triticum macha, Triticum sativum, Triticum monococcum or Triticum vulgare), Tropaeolum minus, Tropaeolum majus, Vaccinium spp., Vida spp., Vigna spp., Viola odorata, Vitis spp., Zea mays, Zizania palustris, Ziziphus spp., amongst others.

### DETAILED DESCRIPTION OF THE INVENTION

### I. LOB-Domain Comprising Protein (LOB: Lateral Organ Boundaries)

Surprisingly, it has now been found that modulating expression in a plant of a nucleic acid encoding a LBD polypeptide gives plants having enhanced yield-related traits relative to control plants. According to a first embodiment, the present invention provides a method for enhancing yield-related traits in plants relative to control plants, comprising modulating expression in a plant of a nucleic acid encoding a LBD polypeptide.

A preferred method for modulating (preferably, increasing) expression of a nucleic acid encoding a LBD polypeptide is by introducing and expressing in a plant a nucleic acid encoding a LBD polypeptide.

Any reference hereinafter to a “protein useful in the methods of the invention” is taken to mean a LBD polypeptide as defined herein. Any reference hereinafter to a “nucleic acid useful in the methods of the invention” is taken to mean a nucleic acid capable of encoding such a LBD polypeptide. The nucleic acid to be introduced into a plant (and therefore useful in performing the methods of the invention) is any nucleic acid encoding the type of protein which will now be described, hereafter also named “LSO nucleic acid” or “LBD gene”.

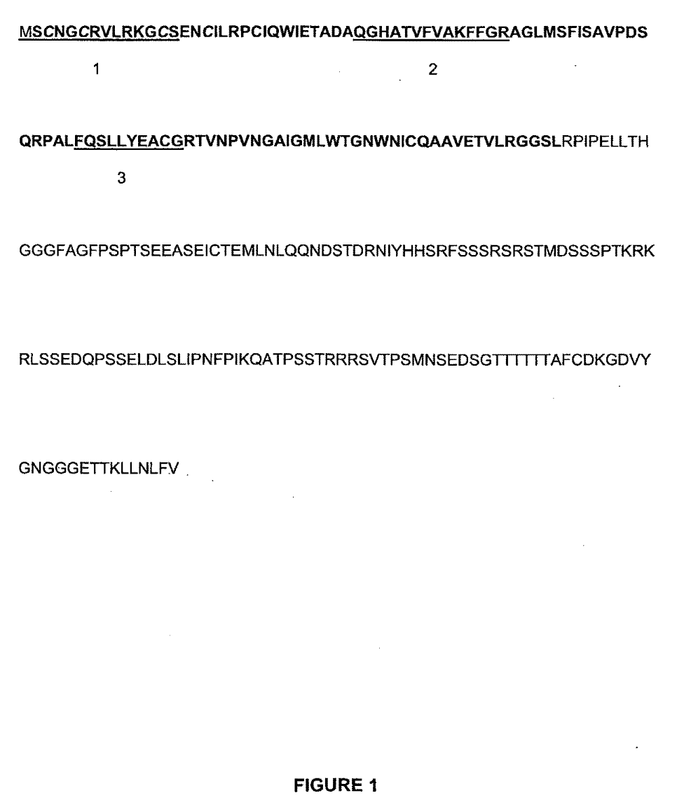

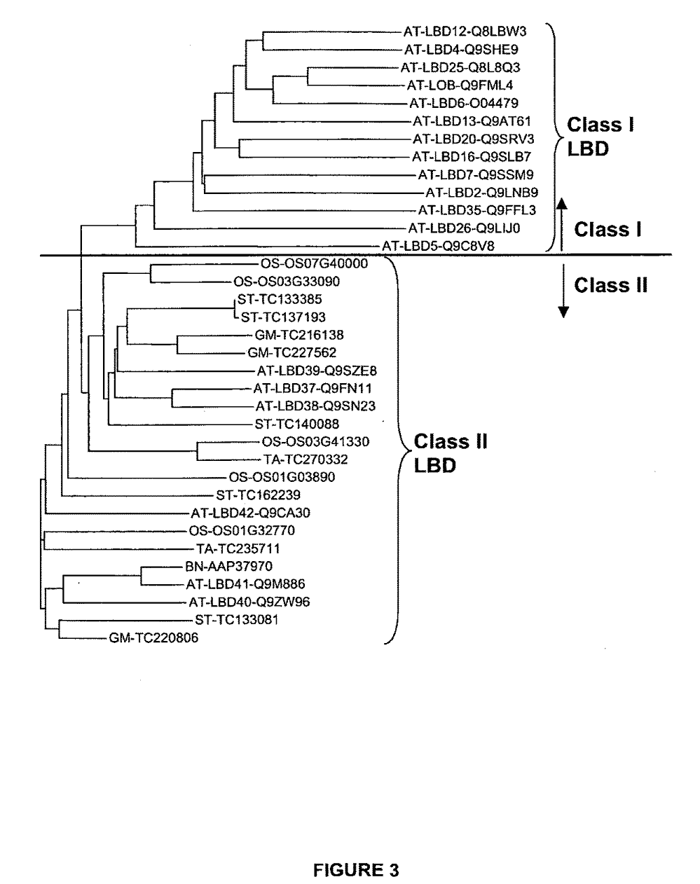

A “LBD polypeptide” as defined herein refers to any polypeptide comprising a DUF260 domain (Pfam accession number PF03195, Interpro accession number IPR004883, see FIG. 1). Preferably, the LBD polypeptide sequence, when used in the construction of a phylogenetic tree such as the one depicted in FIG. 3 (or as defined by Yang et al., 2006, using the HKY method (Hasegawa et al., J. Mol. Evol. 22, 160-174, 1985)), clusters with the group of class II LOB domain polypeptides comprising the amino acid sequence represented by SEQ ID NO: 2 rather than with any other group.

Further preferably, the LBD protein comprises at least one of the following conserved motifs:

Motif 1: MSCNGCRXLRKGCX (SEQ ID NO: 5); wherein X on position 8 may be any amino acid, but preferably V or I and wherein X on position 14 may be any amino acid, but preferably is one of S, G or N.
Motif 2: QXXATXFXAKFXGR (SEQ ID NO: 6), wherein X on position 2 may be any amino acid, but preferably one of A, 5, or G; wherein X on position 3 may be any amino acid, but preferably one of N, Q, or H; wherein X on position 6 may be any amino acid, but preferably one of V, L, or I; wherein X on position 8 may be any amino acid, but preferably one of L, V, A, or I; and wherein X on position 12 may be any amino acid, but preferably one of Y or F.
Motif 3: FXSLLXEAXG (SEQ ID NO: 7); wherein X on position 2 may be any amino acid, but preferably one of R, S, K, or Q; wherein X on position 6 may be any amino acid, but preferably one of Y, H, or F; and wherein X on position 9 may be any amino acid, but preferably C or A.

Further preferably, the LBD protein comprises more than seven, more preferably at least nine Cys residues, and does not comprise the DP(V/I)YG signature (SEQ ID NO: 8).

The DUF260 domain is characterised by the presence of a C-x(2)-C-x(6)-C-x(3)-C motif (SEQ ID NO: 9), wherein X may be any amino acid.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004), or Pfam (Bateman et al., Nucleic Acids Research 30(1): 276-280 (2002). A set of tools for in silica analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, LBD polypeptides (at least in their native form), as transcription factors, typically have DNA binding activity. Tools and techniques for measuring DNA binding activity are well known in the art and include for example gel retardation assays. Experimental approaches for characterising activity of transcription factors may be found for example in Sambrook et al. (2001) Molecular Cloning: a laboratory manual, 3rd Edition Cold Spring Harbor Laboratory Press, CSH, New York, or in Volumes 1 and 2 of Ausubel et al. (yearly updated), Current Protocols in Molecular Biology, Current Protocols.

The present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any LBD-encoding nucleic acid or LBD polypeptide as defined herein.

Examples of nucleic acids encoding LBD polypeptides are given in Table A1 of Example 1 herein. Such nucleic acids are useful in performing the methods of the invention. The amino acid sequences given in Table A1 of Example 1 are example sequences of orthologues and paralogues of the LBD polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A1 of Example 1, the terms “homologue” and “derivative” being as defined herein. Also useful in the methods of the invention are nucleic acids encoding homologues and derivatives of orthologues or paralogues of any one of the amino acid sequences given in Table A1 of Example 1. Homologues and derivatives useful in the methods of the present invention have substantially the same biological and functional activity as the unmodified protein from which they are derived.

Further nucleic acid variants useful in practising the methods of the invention include portions of nucleic acids encoding LBD polypeptides, nucleic acids hybridising to nucleic acids encoding LBD polypeptides, splice variants of nucleic acids encoding LBD polypeptides, allelic variants of nucleic acids encoding LBD polypeptides and variants of nucleic acids encoding LBD polypeptides obtained by gene shuffling. The terms hybridising sequence, splice variant, allelic variant and gene shuffling are as described herein.

Advantageously, the present invention provides hitherto unknown LBD nucleic acid and polypeptide sequences.

According to a further embodiment of the present invention, there is provided an isolated nucleic acid molecule comprising:
(i) a nucleic acid represented by SEQ ID NO: 69;
(ii) a nucleic acid or fragment thereof that is complementary to any one of the SEQ ID NOs given in (i);
(iii) a nucleic acid encoding a LBD polypeptide having, in increasing order of preference, at least 60%, 65%, 70%, 75%, 80%, 85%, 90%, 95%, 96%, 97%, 98%, 99% or more sequence identity to SEQ ID NO: 70;
(iv) a nucleic acid capable of hybridizing under stringent conditions to any one of the nucleic acids given in (i), (ii) or (iii) above.

According to a further embodiment of the present invention, there is therefore provided an isolated polypeptide comprising:
(i) an amino acid sequence having, in increasing order of preference, at least 60%, 65%, 70%, 75%, 80%, 85%, 90%, 95%, 96%, 97%, 98%, 99% or 100% sequence identity to the amino acid sequence given in SEQ ID NO: 70.
(ii) derivatives of any of the amino acid sequences given in (i).

Nucleic acids encoding LBD polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A1 of Example 1, or a portion of a nucleic acid encoding an orthologue, paralogue or homologue of any of the amino acid sequences given in Table A1 of Example 1.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a LBD polypeptide as defined herein, and have substantially the same biological activity as the amino acid sequences given in Table A1 of Example 1. Preferably, the portion is a portion of any one of the nucleic acids given in Table A1 of Example 1, or is a portion of a nucleic acid encoding an orthologue or paralogue of any one of the amino acid sequences given in Table A1 of Example 1. Preferably the portion is at least 500, 550, 600, 650, 700, 750, 800, 850, 900, 950, 1000-consecutive nucleotides in length, the consecutive nucleotides being of any one of the nucleic acid sequences given in Table A1 of Example 1, or of a nucleic acid encoding an orthologue or paralogue of any one of the amino acid sequences given in Table A1 of Example 1. Most preferably the portion is a portion of the nucleic acid of SEQ ID NO: 1. Preferably, the portion encodes an amino acid sequence which when used in the construction of a phylogenetic tree such as the one depicted in FIG. 3 (or as defined by Yang et al., 2006, using the HKY method (Hasegawa et al., J. Mol. Evol. 22, 160-174, 1985)), clusters with the group of class II LOB domain polypeptides comprising the amino acid sequence represented by SEQ ID NO: 2 rather than with any other group.

Another nucleic acid variant useful in the methods of the invention is a nucleic acid capable of hybridising, under reduced stringency conditions, preferably under stringent conditions, with a nucleic acid encoding a LBD polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A1 of Example 1, or comprising introducing and expressing in a plant a nucleic acid capable of hybridising to a nucleic acid encoding an orthologue, paralogue or homologue of any of the nucleic acid sequences given in Table A1 of Example 1.

Hybridising sequences useful in the methods of the invention encode a LBD polypeptide as defined herein, and have substantially the same biological activity as the amino acid sequences given in Table A1 of Example 1. Preferably, the hybridising sequence is capable of hybridising to any one of the nucleic acids given in Table A1 of Example 1, or to a portion of any of these sequences, a portion being as defined above, or wherein the hybridising sequence is capable of hybridising to a nucleic acid encoding an orthologue or paralogue of any one of the amino acid sequences given in Table A1 of Example 1. Most preferably, the hybridising sequence is capable of hybridising to a nucleic acid as represented by SEQ ID NO: 1 or to a portion thereof.

Preferably, the hybridising sequence encodes an amino acid sequence which when used in the construction of a phylogenetic tree such as the one depicted in FIG. 3 (or as defined by Yang at al., 2006, using the HKY method (Hasegawa et al., J. MeI. Evol. 22, 160-174, 1985)), clusters with the group of class II LOB domain polypeptides comprising the amino acid sequence represented by SEQ ID NO: 2 rather than with any other group.

Another nucleic acid variant useful in the methods of the invention is a splice variant encoding a LBD polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A1 of Example 1, or a splice variant of a nucleic acid encoding an orthologue, paralogue or homologue of any of the amino acid sequences given in Table A1 of Example 1.

Preferred splice variants are splice variants of a nucleic acid represented by SEQ ID NO: 1, or a splice variant of a nucleic acid encoding an orthologue or paralogue of SEQ ID NO: 2. Preferably, the amino acid sequence encoded by the splice variant, when used in the construction of a phylogenetic tree such as the one depicted in FIG. 3 (or as defined by Yang at al., 2006, using the HKY method (Hasegawa et al., J. Mol. Evol. 22, 160-174, 1985)), clusters with the group of class II LOB domain polypeptides comprising the amino acid sequence represented by SEQ ID NO: 2 rather than with any other group.

Another nucleic acid variant useful in performing the methods of the invention is an allelic variant of a nucleic acid encoding a LBD polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table A1 of Example 1, or comprising introducing and expressing in a plant an allelic variant of a nucleic acid encoding an orthologue, paralogue or homologue of any of the amino acid sequences given in Table A1 of Example 1.

The allelic variants useful in the methods of the present invention have substantially the same biological activity as the LBD polypeptide of SEQ ID NO: 2 and any of the amino acids depicted in Table A1 of Example 1. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant, when used in the construction of a phylogenetic tree such as the one depicted in FIG. 3 (or as defined by Yang et al., 2006, using the HKY method (Hasegawa et al., J. Mol. Evol. 22, 160-174, 1985)), clusters with the group of class II LOB domain polypeptides comprising the amino acid sequence represented by SEQ ID NO: 2 rather than with any other group.

Gene shuffling or directed evolution may also be used to generate variants of nucleic acids encoding LBD polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table A1 of Example 1, or comprising introducing and expressing in a plant a variant of a nucleic acid encoding an orthologue, paralogue or homologue of any of the amino acid sequences given in Table A1 of Example 1, which variant nucleic acid is obtained by gene shuffling.

Preferably, the amino acid sequence encoded by the variant nucleic acid obtained by gene shuffling, when used in the construction of a phylogenetic tree such as the one depicted in FIG. 3 (or as defined by Yang et al., 2006, using the HKY method (Hasegawa et al., J. Mol. Evol. 22, 160-174, 1985)), clusters with the group of class II LOB domain polypeptides comprising the amino acid sequence represented by SEQ ID NO: 2 rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding LBD polypeptides may be derived from any natural or artificial source. The nucleic acid may be modified from its native form in composition and/or genomic environment through deliberate human manipulation. Preferably the LBD polypeptide-encoding nucleic acid is from a plant, further preferably from a dicotyledonous plant, more preferably from the family Brassicaceae, most preferably the nucleic acid is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such aboveground harvestable parts are shoot biomass and seeds, and performance of the methods of the invention results in plants having increased shoot biomass and seed yield relative to the shoot biomass and seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially shoot biomass yield and seed yield of plants, relative to control plants, which method comprises modulating expression, preferably increasing expression, in a plant of a nucleic acid encoding a LBD polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a LBD polypeptide as defined herein. In particular, an increase in growth rate was observed during the early growth stages of the plant (early vigour).

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a LBD polypeptide.

Performance of the methods of the invention gives plants grown under conditions of nutrient deficiency, particularly under conditions of nitrogen deficiency, increased early vigour and increased yield relative to control plants grown under comparable conditions. Therefore, according to the present invention, there is provided a method for increasing early vigour and yield in plants grown under conditions of nutrient deficiency, which method comprises increasing expression in a plant of a nucleic acid encoding a LBD polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a LBD polypeptide as defined above.

The invention also provides genetic constructs and vectors to facilitate introduction and/or expression in plants of nucleic acids encoding LBD polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a LBD polypeptide as defined above;
(b) one or more control sequences capable of driving expression of the nucleic acid sequence of (a); and optionally
(c) a transcription termination sequence.

Preferably, the nucleic acid encoding a LBD polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the LBD polypeptide-encoding nucleic acid represented by SEQ ID NO: 1, nor is the applicability of the invention restricted to expression of a LBD polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 10, most preferably the constitutive promoter is as represented by SEQ ID NO: 10. See the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits relative to control plants, comprising introduction and expression in a plant of any nucleic acid encoding a LBD polypeptide as defined hereinabove.

More specifically, the present invention provides a method for the production of transgenic plants having increased enhanced yield-related traits, particularly increased shoot biomass and increased seed yield, which method comprises:
(i) introducing and expressing in a plant or plant cell a LBD polypeptide-encoding nucleic acid; and
(ii) cultivating the plant cell under conditions promoting plant growth and development.

The nucleic acid of (i) may be any of the nucleic acids capable of encoding a LBD polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hagen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a LBD polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a LBD polypeptide is by introducing and expressing in a plant a nucleic acid encoding a LBD polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding LBD polypeptides as described herein and use of these LBD polypeptides in enhancing any of the aforementioned yield-related traits in plants.

Nucleic acids encoding LBD polypeptide described herein, or the LBD polypeptides themselves, may find use in breeding programmes in which a DNA marker is identified which may be genetically linked to a LBD polypeptide-encoding gene. The nucleic acids/genes, or the LAD polypeptides themselves may be used to define a molecular marker. This DNA or protein marker may then be used in breeding programmes to select plants having enhanced yield-related traits as defined hereinabove in the methods of the invention.

Allelic variants of a LBD polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding LAD polypeptides may also be used as probes for genetically and physically mapping the genes that they are a part of, and as markers for traits linked to those genes. Such information may be useful in plant breeding in order to develop lines with desired phenotypes. Such use of LAD polypeptide-encoding nucleic acids requires only a nucleic acid sequence of at least 15 nucleotides in length. The LAD polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the LBD-encoding nucleic acids. The resulting banding patterns may then be subjected to genetic analyses using computer programs such as MapMaker (Lander at al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the LBD polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Lean et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### II. JMJC (JUMONJI-C) Polypeptide

Surprisingly, it has now been found that modulating expression in a plant of a nucleic acid encoding a JMJC polypeptide gives plants having enhanced yield-related traits relative to control plants. According to a first embodiment, the present invention provides a method for enhancing yield-related traits in plants relative to control plants, comprising modulating expression in a plant of a nucleic acid encoding a JMJC polypeptide.

A preferred method for modulating (preferably, increasing) expression of a nucleic acid encoding a JMJC polypeptide is by introducing and expressing in a plant a nucleic acid encoding a JMJC polypeptide.

Any reference hereinafter to a “protein useful in the methods of the invention” is taken to mean a JMJC polypeptide as defined herein. Any reference hereinafter to a “nucleic acid useful in the methods of the invention” is taken to mean a nucleic acid capable of encoding such a JMJC polypeptide. The nucleic acid to be introduced into a plant (and therefore useful in performing the methods of the invention) is any nucleic acid encoding the type of protein, which will now be described, hereafter also named “JMJC nucleic acid” or “JMJC gene”.

A JMJC polypeptide as defined herein refers to a polypeptide comprising at least a JmjC domain.

The JmjC domain is a conserved sequence found in proteins of prokaryotic and eukaryotic organisms. JmjC domains are in average 111 amino acids long. Typically the length of a JmjC domain can range between 25 to 200 amino acids though shorter and longer versions maybe possible. JmjC domains are predicted to be metalloenzymes that adopt the cupin fold, and are candidates for enzymes that regulate chromatin remodelling (Clissold et al. Trends Biochem Sci. 2001 January; 26(1):7-9).

JMJC polypeptides can be found in specialized databases such as Pfam, (Finn et al. Nucleic Acids Research (2006) Database Issue 34:D247-D251). Pfam compiles a large collection of multiple sequence alignments and hidden Markov models (HMM) covering many common protein domains and families and is available through the Sanger Institute in the United Kingdom.

The gathering cutoff threshold of the JmjC domain in the Pfam HMM_fs model is of 16.0 and of −8.0 in the HMM_ls model. Trusted matches as considered in the Pfam database are those scoring higher than the gathering cut-off threshold. However potential matches, comprising true JmjC domains, may still fall under the gathering cut-off. Preferably a JMJC polypeptide is a protein having one or more domains in their sequence that exceed the gathering cutoff of the Pfam protein domain family PF02373, jumonji, jmjC,

Alternatively, a JmjC domain in a polypeptide may be identified by performing a sequence comparison with known polypeptides comprising a JmjC domain and establishing the similarity in the region of the JmjC domain. The sequences may be aligned using any of the methods well known in the art such as Blast algorithms and the probability for the alignment to occur with a given sequence is taken as basis for identifying similar polypeptides. A parameter that is typically used to represent such probability in pair wise comparisons is called e-value. The e-value describes how often a given score is expected to occur random; The e-value cut-off may be as high as 1.0. Typically alignments with high likelihood to occur have an e-value lower than 0.1, 0.01, 0.001, 1.e-05, 1.e-10, 1.e-15, 1.e-20, 1.e-25, 1.e-50, 1.e-75, 1.e-100 and 1.e-200. Preferably JMJC polypeptides of the invention comprise a sequence having in increasing order of preference an e-value lower than 0.1, 0.01, 0.001, 1.e-05, 1.e-10, 1.e-15, 1.e-20, 1.e-25, 1.e-50, 1.e-75, 1.e-100 and 1.e-200 for an alignment with JmjC domain found in a known JMJC polypeptide.

Examples of JMJC polypeptides are given in Table B1. The amino acid coordinates of the JmjC domains as present in representative JMJC polypeptides of dicotyledonous origin is given in Table B4. An example of JmjC domain is given in SEQ ID NO: 78.

The sequence identity between JmjC domains is typically low, in average 23% and maybe as low as 10%. Preferred JMJC polypeptides of the inventions are those having in increasing order of preference at least 50%, 55%, 60%, 65%, 70%, 75%, 80%, 85%, 90%, 95%, or more sequence identity to SEQ ID NO: 78 or to any of the JmjC domains comprised in the JMJC polypeptides represented by SEQ ID NO: 84; SEQ ID NO: 86; SEQ ID NO: 96; SEQ ID NO: 98; SEQ ID NO: 104; SEQ ID NO: 108; SEQ ID NO: 110; SEQ ID NO: 112; SEQ ID NO: 114; SEQ ID NO: 116; SEQ ID NO: 118; SEQ ID NO: 120; SEQ ID NO: 122; SEQ ID NO: 124; SEQ ID NO: 128; SEQ ID NO: 130; SEQ ID NO: 132; and SEQ ID NO: 134, whose amino acid coordinates are given in Table B4.

In addition to the JmjC domain, JMJC polypeptides may optionally comprise other highly conserved sequence motifs, such as those represented in SEQ ID NO: 79, SEQ ID NO: 80, SEQ ID NO: 81, which are found in SEQ ID NO: 74. Further, the polypeptides of the invention may comprise a conserved sequence found in oxygenases which has been involved in the coordination of iron cations, herein represented by HXD(V) or EXnH (SEQ ID NO: 82), wherein “X” represents any amino acid and “n” represents the number of X-residues ranging between 1-5, both included.

A preferred JMJC polypeptide of the invention refers to any polypeptide comprising a JmjC domain and optionally having in increasing order of preference at least 50%, 55%, 60%, 65%, 70%, 75%, 80%, 85%, 90%, 95% or more sequence identity to any one or more of the conserved sequence motifs as represented by SEQ ID NO: 79, SEQ ID NO: 80, SEQ ID NO: 81 and SEQ ID NO: 82.

Evidence of domain swapping in the JUMONJI family of proteins has been reported, (Balciunas and Ronne. Trends Biochem Sci 2000; 25:274-276). Accordingly, in addition to the JmjC domain, JMJC polypeptides typically contain one or more different kinds of other known conserved domains. Relevant to the polypeptides of the invention are JMJC polypeptides that in addition to the JmjC domain comprise other conserved domains, which are presumably involved in DNA binding and transcription activities such as zinc fingers and/or in protein interaction and protein turnover, such as F-box and zinc finger-RING-type domains.

Therefore the JMJC polypeptide useful in the methods of the invention comprises a JmjC domain and optionally one or more of the following conserved domains: JmJN (pfam accession number PF02375); C5HC2 zinc finger (pfam accession number: PF02928), FY-rich domain, N-terminal (InterPro accession number: IPR003888) and FY-rich domain, C-terminal (InterPro accession number: IPR003889), a Zinc finger FYVE/PHD-Zn type (InterPro accession number: IPR011011), a Zinc finger C₂H₂ type (pfam accession number: PF00096), a Zinc finger —RING type (zf-C3HC4) (pfam accession number PF00097) and an F-box domain (pfam accession number: PF00646).

Preferably the JMJC polypeptide of the invention comprises a sequence having in increasing order of preference of 50%, 60%, 70%, 75%, 60%, 85%, 90%, 92%, 94%, 96%, 98% or more sequence identity to SEQ ID NO: 74, SEQ ID NO: 86, SEQ ID NO: 94, SEQ ID NO: 104, SEQ ID NO: 122, SEQ ID NO: 134, SEQ ID NO: 136, SEQ ID NO: 140, SEQ ID NO: 142 and SEQ ID NO: 148.

Preferably, the polypeptide sequence which when used in the construction of a phylogenetic tree, such as the one depicted in FIG. 8, clusters with the group of JMJC polypeptides (Takeuchi et al. Dev Dyn. 2006 235(9): 2449-59) comprising the amino acid sequence represented by SEQ ID NO: 74 rather than with any other group.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004), or Pfam (Bateman et al., Nucleic Acids Research 30(1): 276-280 (2002). A set of tools for in silica analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mal Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, JMJC polypeptides may typically have protein hydroxylases activity, in particular peptide-aspartate beta-dioxygenase (PABD) activity (EC 1.14.11.16). The reaction catalyzed is: peptide L.-aspartate+2-oxoglutarate+O(2)<=>peptide 3-hydroxy-L-aspartate+succinate+CO(2). The cofactors in the reaction are iron and 2-Oxogluatarate. Tools and techniques for measuring PABD activity are well known in the art (see for example Lavaissiere et al. J Clin Invest. 1996; 98(6):1313-23; Linke et al. J Biol. Chem. 2004; 279(14):14391-7; Lee, et al. J. Biol. Chem. 278:7558-7563; 2003; Lando et al. Genes Dev. 16:1466-1471; 2002). Iron (11)12-oxoglutarate (2-OG)-dependent oxygenases catalyse oxidative reactions in various metabolic reactions. Recently it has been proposed that JMJC polypeptides can also have histone demethylase activity (Trewick et al. 2005).

The present invention is illustrated by transforming plants with the nucleic acid sequence represented by SEQ ID NO: 73, encoding the polypeptide sequence of SEQ ID NO: 74. However, performance of the invention is not restricted to these sequences; the methods of the invention may advantageously be performed using any JMJC-encoding nucleic acid or JMJC polypeptide as defined herein.

Examples of nucleic acids encoding JMJC polypeptides are given in Table B1 of Example 12 herein. Such nucleic acids are useful in performing the methods of the invention. The amino acid sequences given in Table B1 of Example 12 are example sequences of orthologues and paralogues of the JMJC polypeptide represented by SEQ ID NO: 74, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table 81 of Example 12) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 73 or SEQ ID NO: 74, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table B1 of Example 12, the terms “homologue” and “derivative” being as defined herein. Also useful in the methods of the invention are nucleic acids encoding homologues and derivatives of orthologues or paralogues of any one of the amino acid sequences given in Table B1 of Example 12. Homologues and derivatives useful in the methods of the present invention have substantially the same biological and functional activity as the unmodified protein from which they are derived.

Further nucleic acid variants useful in practising the methods of the invention include portions of nucleic acids encoding JMJC polypeptides, nucleic acids hybridising to nucleic acids encoding JMJC polypeptides, splice variants of nucleic acids encoding JMJC polypeptides, allelic variants of nucleic acids encoding JMJC polypeptides and variants of nucleic acids encoding JMJC polypeptides obtained by gene shuffling. The terms hybridising sequence, splice variant, allelic variant and gene shuffling are as described herein.

Nucleic acids encoding JMJC polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table B1 of Example 12, or a portion of a nucleic acid encoding an orthologue, paralogue or homologue of any of the amino acid sequences given in Table B1 of Example 12.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a JMJC polypeptide as defined herein, and have substantially the same biological activity as the amino acid sequences given in Table B1 of Example 12. Preferably, the portion is a portion of any one of the nucleic acids given in Table B1 of Example 12, or is a portion of a nucleic acid encoding an orthologue or paralogue of any one of the amino acid sequences given in Table B1 of Example 12. Preferably the portion is at least 100, 200, 500, 550, 600, 650, 700, 750, 800, 850, 900, 950, 1000, 1500, 2000, 2500, 3000, 3500, 4000, 4500, 5000 consecutive nucleotides in length, the consecutive nucleotides being of any one of the nucleic acid sequences given in Table B1 of Example 12, or of a nucleic acid encoding an orthologue or paralogue of any one of the amino acid sequences given in Table B1 of Example 12. Most preferably the portion is a portion of the nucleic acid of SEQ ID NO: 73. Preferably, the portion encodes an amino acid sequence which when used in the construction of a phylogenetic tree, such as the one depicted in FIG. 8, clusters with the group of JMJC polypeptides (Takeuchi et al. rev Dyn. 2006 235(9):2449-59) comprising the amino acid sequence represented by SEQ ID NO: 74 rather than with any other group.

Another nucleic acid variant useful in the methods of the invention is a nucleic acid capable of hybridising, under reduced stringency conditions, preferably under stringent conditions, with a nucleic acid encoding a JMJC polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table B1 of Example 12, or comprising introducing and expressing in a plant a nucleic acid capable of hybridising to a nucleic acid encoding an orthologue, paralogue or homologue of any of the nucleic acid sequences given in Table B1 of Example 12.

Hybridising sequences useful in the methods of the invention encode a JMJC polypeptide as defined herein, and have substantially the same biological activity as the amino acid sequences given in Table B1 of Example 12. Preferably, the hybridising sequence is capable of hybridising to any one of the nucleic acids given in Table B1 of Example 12, or to a portion of any of these sequences, a portion being as defined above; or wherein the hybridising sequence is capable of hybridising to a nucleic acid encoding an orthologue or paralogue of any one of the amino acid sequences given in Table B1 of Example 12. Most preferably, the hybridising sequence is capable of hybridising to a nucleic acid as represented by SEQ ID NO: 73 or to a portion thereof.

Preferably, the hybridising sequence encodes an amino acid sequence which when used in the construction of a phylogenetic tree, such as the one depicted in FIG. 8, clusters with the group of JMJC polypeptides (Takeuchi et al. Dev Dyn. 2006 235(9):2449-59) comprising the amino acid sequence represented by SEQ ID NO: 74 rather than with any other group.

Another nucleic acid variant useful in the methods of the invention is a splice variant encoding a JMJC polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table B1 of Example 12, or a splice variant of a nucleic acid encoding an orthologue, paralogue or homologue of any of the amino acid sequences given in Table B1 of Example 12. An example of a spliced variant of the gene encoding SEQ ID NO: 74 is represented by SEQ ID NO: 73 and SEQ ID NO: 83 (encoding SEQ ID NO: 84).

Preferred splice variants are splice variants of a nucleic acid represented by SEQ ID NO: 73, or a splice variant of a nucleic acid encoding an orthologue or paralogue of SEQ ID NO: 74. Preferably, the amino acid sequence encoded by the splice variant, when used in the construction of a phylogenetic tree, such as the one depicted in FIG. 8, clusters with the group of JMJC polypeptides (Takeuchi et al. Dev Dyn. 2006 235(9):2449-59) comprising the amino acid sequence represented by SEQ ID NO: 74 any other group.

Another nucleic acid variant useful in performing the methods of the invention is an allelic variant of a nucleic acid encoding a JMJC polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table B1 of Example 12, or comprising introducing and expressing in a plant an allelic variant of a nucleic acid encoding an orthologue, paralogue or homologue of any of the amino acid sequences given in Table B1 of Example 12.

The allelic variants useful in the methods of the present invention have substantially the same biological activity as the JMJC polypeptide of SEQ ID NO: 74 and any of the amino acids depicted in Table B1 of Example 12. Allelic variants exist in nature, and encompassed within the methods of the present invention is the use of these natural alleles. Preferably, the allelic variant is an allelic variant of SEQ ID NO: 73 or an allelic variant of a nucleic acid encoding an orthologue or paralogue of SEQ ID NO: 74. Preferably, the amino acid sequence encoded by the allelic variant, when used in the construction of a phylogenetic tree, such as the one depicted in FIG. 8, clusters with the JMJC polypeptides (Takeuchi et al. Dev Dyn. 2006 235(9):2449-59) comprising the amino acid sequence represented by SEQ ID NO: 74 rather than with any other group.

Gene shuffling or directed evolution may also be used to generate variants of nucleic acids encoding JMJC polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table B1 of Example 12, or comprising introducing and expressing in a plant a variant of a nucleic acid encoding an orthologue, paralogue or homologue of any of the amino acid sequences given in Table B1 of Example 12, which variant nucleic acid is obtained by gene shuffling.

Advantageously, the present invention provides hitherto unknown JMJ nucleic acid and polypeptide sequences.

According to a further embodiment of the present invention, there is provided an isolated nucleic acid molecule comprising:
(i) a nucleic acid represented by SEQ ID NO: 169;
(ii) a nucleic acid or fragment thereof that is complementary to SEQ ID NO: 169;
(iii) a nucleic acid encoding an JMJC polypeptide having, in increasing order of preference, at least 70%, 75%, 80%, 85%, 90%, 95%, 96%, 97%, 98%, 99% or more sequence identity to the amino acid sequence given in SEQ ID NO: 170;
(iv) a nucleic acid capable of hybridizing under stringent conditions to any one of the nucleic acids given in (i), (ii) or (iii) above.

According to a further embodiment of the present invention, there is therefore provided an isolated polypeptide comprising:
(i) an amino acid sequence having, in increasing order of preference, at least 70%, 75%, 80%, 85%, 90%, 95%, 96%, 97%, 98%, 99% or 100% or more sequence identity to the amino acid sequence given in SEQ ID NO: 170;
(ii) derivatives of any of the amino acid sequences given in (i).

Preferably, the amino acid sequence encoded by the variant nucleic acid obtained by gene shuffling, when used in the construction of a phylogenetic tree such as the one depicted in FIG. 8, clusters with the group of JMJC polypeptides (Takeuchi et al. Dev Dyn. 2006 235(9):2449-59) comprising the amino acid sequence represented by SEQ ID NO: 74 rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding JMJC polypeptides may be derived from any natural or artificial source. The nucleic acid may be modified from its native form in composition and/or genomic environment through deliberate human manipulation. Preferably the JMJC polypeptide-encoding nucleic acid is from a plant, further preferably from a dicotyledonous plant, more preferably from the family Brassicaceae, most preferably the nucleic acid is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased yield relative to the yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a JMJC polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a JMJC polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a JMJC polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a JMJC polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a JMJC polypeptide as defined above.

The invention also provides genetic constructs and vectors to facilitate introduction and/or expression in plants of nucleic acids encoding JMJC polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a JMJC polypeptide as defined above;
(b) one or more control sequences capable of driving expression of the nucleic acid sequence of (a); and optionally
(c) a transcription termination sequence.

Preferably, the nucleic acid encoding a JMJC polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the JMJC polypeptide-encoding nucleic acid represented by SEQ ID NO: 73, nor is the applicability of the invention restricted to expression of a JMJC polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 75, most preferably the constitutive promoter is as represented by SEQ ID NO: 75. See the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits relative to control plants, comprising introduction and expression in a plant of any nucleic acid encoding a JMJC polypeptide as defined hereinabove.

More specifically, the present invention provides a method for the production of transgenic plants having increased enhanced yield-related traits, particularly increased (seed) yield, which method comprises:
(i) introducing and expressing in a plant or plant cell a JMJC polypeptide-encoding nucleic acid; and
(ii) cultivating the plant cell under conditions promoting plant growth and development.

The nucleic acid of (i) may be any of the nucleic acids capable of encoding a JMJC polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a JMJC polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a JMJC polypeptide is by introducing and expressing in a plant a nucleic acid encoding a JMJC polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding JMJC polypeptides as described herein and use of these JMJC polypeptides in enhancing any of the aforementioned yield-related traits in plants.

Nucleic acids encoding JMJC polypeptides described herein, or the JMJC polypeptides themselves, may find use in breeding programmes in which a DNA marker is identified which may be genetically linked to a JMJC polypeptide-encoding gene. The nucleic acids/genes, or the JMJC polypeptides themselves may be used to define a molecular marker. This DNA or protein marker may then be used in breeding programmes to select plants having enhanced yield-related traits as defined hereinabove in the methods of the invention.

Allelic variants of a JMJC polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding JMJC polypeptides may also be used as probes for genetically and physically mapping the genes that they are a part of, and as markers for traits linked to those genes. Such information may be useful in plant breeding in order to develop lines with desired phenotypes. Such use of JMJC polypeptide-encoding nucleic acids requires only a nucleic acid sequence of at least 15 nucleotides in length. The JMJC polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the JMJC-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the JMJC polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al, (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### III. CKI (Casein Kinase I) Polypeptide

Surprisingly, it has now been found that modulating expression in a plant of a nucleic acid encoding a CKI polypeptide gives plants having enhanced yield-related traits relative to control plants, According to a first embodiment, the present invention provides a method for enhancing yield-related traits in plants relative to control plants, comprising modulating expression in a plant of a nucleic acid encoding a CKI polypeptide.

A preferred method for modulating (preferably, increasing) expression of a nucleic acid encoding a CKI polypeptide is by introducing and expressing in a plant a nucleic acid encoding a CKI polypeptide.

Any reference hereinafter to a “protein useful in the methods of the invention” is taken to mean a CKI polypeptide as defined herein. Any reference hereinafter to a “nucleic acid useful in the methods of the invention” is taken to mean a nucleic acid capable of encoding such a CKI polypeptide. The nucleic acid to be introduced into a plant (and therefore useful in performing the methods of the invention) is any nucleic acid encoding the type of protein which will now be described, hereafter also named “CKI nucleic acid” or “CKI gene”.

A “CKI polypeptide” as defined herein refers the proteins represented by SEQ ID NO: 174 and to homologues (orthologues and paralogues) thereof. Preferably, the homologues of SEQ ID NO: 174 have a casein kinase domain.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hula et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, CKI polypeptides, as far as SEQ ID NO: 174 and its homologues are concerned, the CKI proteins useful in the methods in the methods of the present invention typically have kinase activity. Methods for measuring kinase activity are known in the art.

The present invention is illustrated by transforming plants with the nucleic acid sequence represented by SEQ ID NO: 173, encoding the polypeptide sequence of SEQ ID NO: 174 respectively. However, performance of the invention is not restricted to these sequences; the methods of the invention may advantageously be performed using any CKI-encoding nucleic acid or CKI polypeptide as defined herein.

Examples of nucleic acids encoding CKI polypeptides may be found in databases known in the art. Such nucleic acids are useful in performing the methods of the invention. Orthologues and paralogues, the terms “orthologues” and “paralogues” being as defined herein, may readily be identified by performing a so-called reciprocal blast search. Typically, this involves a first BLAST involving BLASTing a query sequence (for example using SEQ ID NO: 174) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 173 or SEQ ID NO: 174, the second BLAST would therefore be against Nicotiana tabacum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants encoding homologues and derivatives of SEQ ID NO: 174 may also be useful in practising the methods of the invention, the terms “homologue” and “derivative” being as defined herein. Also useful in the methods of the invention are nucleic acids encoding homologues and derivatives of orthologues or paralogues of SEQ ID NO: 174. Homologues and derivatives useful in the methods of the present invention have substantially the same biological and functional activity as the unmodified protein from which they are derived.

Further nucleic acid variants useful in practising the methods of the invention include portions of nucleic acids encoding CKI polypeptides, nucleic acids hybridising to nucleic acids encoding CKI polypeptides, splice variants of nucleic acids encoding CKI polypeptides, allelic variants of nucleic acids encoding CKI polypeptides and variants of nucleic acids encoding CKI polypeptides obtained by gene shuffling. The terms hybridising sequence, splice variant, allelic variant and gene shuffling are as described herein.

Nucleic acids encoding CKI polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of SEQ ID NO: 173, or a portion of a nucleic acid encoding an orthologue, paralogue or homologue of SEQ ID NO: 174.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a CKI polypeptide as defined herein, and have substantially the same biological activity as the amino acid sequences given in SEQ ID NO: 174. Preferably, the portion is a portion of any one of the nucleic acids given in SEQ ID NO: 173, or is a portion of a nucleic acid encoding an orthologue or paralogue of any one of the amino acid sequences given in SEQ ID NO: 173. Preferably the portion is at least 500, 550, 600, 650, 700, 750, 800, 850, 900, 950, 1000, 1200, 1300, 1400 consecutive nucleotides in length, the consecutive nucleotides being of SEQ ID NO: 173, or of a nucleic acid encoding an orthologue or paralogue of SEQ ID NO: 174. Most preferably the portion is a portion of the nucleic acid of SEQ ID NO: 173.

Another nucleic acid variant useful in the methods of the invention is a nucleic acid capable of hybridising, under reduced stringency conditions, preferably under stringent conditions, with a nucleic acid encoding a CKI polypeptide as defined herein, or with a portion as defined herein.

According to the present invention, there is provided a method for enhancing yield-related traits in plants, comprising introducing and expressing in a plant a nucleic acid capable of hybridizing to SEQ ID NO: 173, or comprising introducing and expressing in a plant a nucleic acid capable of hybridising to a nucleic acid encoding an orthologue, paralogue or homologue of SEQ ID NO: 173.

Hybridising sequences useful in the methods of the invention encode a CKI polypeptide as defined herein, having substantially the same biological activity as the amino acid sequences given in SEQ ID NO: 174. Preferably, the hybridising sequence is capable of hybridising to SEQ ID NO: 173, or to a portion of any of these sequences, a portion being as defined above, or the hybridising sequence is capable of hybridising to a nucleic acid encoding an orthologue or paralogue of SEQ ID NO: 174.

Another nucleic acid variant useful in the methods of the invention is a splice variant encoding a CKI polypeptide as defined hereinabove, a splice variant being as defined herein.

According to the present invention, there is provided a method for enhancing yield-related traits in plants, comprising introducing and expressing in a plant a splice variant of SEQ ID NO: 173, or a splice variant of a nucleic acid encoding an orthologue, paralogue or homologue of SEQ ID NO: 174.

Another nucleic acid variant useful in performing the methods of the invention is an allelic variant of a nucleic acid encoding a CKI polypeptide as defined hereinabove, an allelic variant being as defined herein.

According to the present invention, there is provided a method for enhancing yield-related traits in plants, comprising introducing and expressing in a plant an allelic variant of SEQ ID NO: 173, or comprising introducing and expressing in a plant an allelic variant of a nucleic acid encoding an orthologue, paralogue or homologue of the amino acid sequences represented by SEQ ID NO: 174.

The allelic variants useful in the methods of the present invention have substantially the same biological activity as the CKI polypeptide of SEQ ID NO: 174. Allelic variants exist in nature, and encompassed within the methods of the present invention is the use of these natural alleles. Gene shuffling or directed evolution may also be used to generate variants of nucleic acids encoding CKI polypeptides as defined above; the term “gene shuffling” being as defined herein.

According to the present invention, there is provided a method for enhancing yield-related traits in plants, comprising introducing and expressing in a plant a variant of SEQ ID NO: 173, or comprising introducing and expressing in a plant a variant of a nucleic acid encoding an orthologue, paralogue or homologue of SEQ ID NO: 174, which variant nucleic acid is obtained by gene shuffling.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding CKI polypeptides may be derived from any natural or artificial source. The nucleic acid may be modified from its native form in composition and/or genomic environment through deliberate human manipulation. Preferably the CKI polypeptide-encoding nucleic acid is from a plant. In the case of SEQ ID NO: 173, the CKI polypeptide encoding nucleic acid is preferably from a dicotyledonous plant, more preferably from the family Solanaceae, most preferably the nucleic acid is from Nicotiana tabacum.

Performance of the methods of the invention gives plants having enhanced yield-related traits. In particular performance of the methods of the invention gives plants having increased early vigour and increased yield, especially increased biomass and increased seed yield relative to control plants. The terms “yield” and “seed yield” are described in more detail in the “definitions” section herein.

Reference herein to enhanced yield-related traits is taken to mean an increase in early vigour and/or in biomass (weight) of one or more parts of a plant, which may include aboveground (harvestable) parts and/or (harvestable) parts below ground. In particular, such harvestable parts are biomass and/or seeds, and performance of the methods of the invention results in plants having increased early vigour, biomass and/or seed yield relative to the early vigour, biomass or seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially biomass and/or seed yield of plants, relative to control plants, which method comprises modulating expression, preferably increasing expression, in a plant of a nucleic acid encoding a CKI polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a CKI polypeptide as defined herein. In a particular embodiment, performance of the methods of the present invention gives plants with increased early vigour.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield and/or increased early vigour, relative to control plants grown under comparable conditions. Therefore, according to the present invention, there is provided a method for increasing yield and/or early vigour in plants grown under non-stress conditions or under mild drought conditions, which method comprises increasing expression in a plant of a nucleic acid encoding a CKI polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a CKI polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a CKI polypeptide as defined above.

The invention also provides genetic constructs and vectors to facilitate introduction and/or expression in plants of nucleic acids encoding CKI polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a CKI polypeptide as defined above;
(b) one or more control sequences capable of driving expression of the nucleic acid sequence of (a); and optionally
(c) a transcription termination sequence.

Preferably, the nucleic acid encoding a CKI polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A seed specific promoter is particularly useful in the methods of the invention, preferably, the promoter is an embryo specific promoter. See the “Definitions” section herein for definitions of the various promoter types. Also useful in the methods of the invention is a constitutive promoter.

It should be clear that the applicability of the present invention is not restricted to the CKI polypeptide-encoding nucleic acid represented by SEQ ID NO: 173, nor is the applicability of the invention restricted to expression of a CKI polypeptide-encoding nucleic acid when driven by a seed specific promoter, or when driven by a constitutive promoter.

The seed specific promoter is preferably a WSI18 promoter, preferably a WSI18 promoter from rice. Further preferably the seed specific promoter is represented by a nucleic acid sequence substantially similar to SEQ ID NO: 175, most preferably the seed specific promoter is as represented by SEQ ID NO: 175. See the “Definitions” section herein for further examples of seed specific promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a CKI polypeptide as defined hereinabove.

More specifically, the present invention provides a method for the production of transgenic plants having increased enhanced yield-related traits, particularly increased early vigour and/or increased yield, which method comprises:
(i) introducing and expressing in a plant or plant cell a CKI polypeptide-encoding nucleic acid; and
(ii) cultivating the plant cell under conditions promoting plant growth and development.

The nucleic acid of (i) may be any of the nucleic acids capable of encoding a CKI polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a CKI polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a CKI polypeptide is by introducing and expressing in a plant a nucleic acid encoding a CKI polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding CKI polypeptides as described herein and use of these CKI polypeptides in enhancing any of the aforementioned yield-related traits in plants.

Nucleic acids encoding CKI polypeptide described herein, or the CKI polypeptides themselves, may find use in breeding programmes in which a DNA marker is identified which may be genetically linked to a CKI polypeptide-encoding gene. The nucleic acids/genes, or the CKI polypeptides themselves may be used to define a molecular marker. This DNA or protein marker may then be used in breeding programmes to select plants having enhanced yield-related traits as defined hereinabove in the methods of the invention.

Allelic variants of a CKI polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding CKI polypeptides may also be used as probes for genetically and physically mapping the genes that they are a part of, and as markers for traits linked to those genes. Such information may be useful in plant breeding in order to develop lines with desired phenotypes. Such use of CKI polypeptide-encoding nucleic acids requires only a nucleic acid sequence of at least 15 nucleotides in length. The CKI polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the CKI-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the CKI polypeptide-encoding nucleic acid in the genetic map previously obtained using this population (Botstein et al. (1980) Am. J. Hum. Genet, 32:314-331).

The production and use of plant gene-derived probes for use in genetic mapping is described in Bernatzky and Tanksley (1986) Plant Mal. Biol, Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Lean et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### IV. Plant Homeodomain Finger-Homeodomain (PHDf-HD) Polypeptide

Surprisingly, it has now been found that modulating, preferably increasing, expression in a plant of a nucleic acid sequence encoding a PHDf-HD polypeptide gives plants having enhanced yield-related traits, preferably enhanced seed yield-related traits, relative to control plants. According to a first embodiment, the present invention provides a method for enhancing yield-related traits, preferably enhancing seed yield-related traits, in plants relative to control plants, comprising modulating, preferably increasing, expression in a plant of a nucleic acid sequence encoding a PHDf-HD polypeptide.

A preferred method for modulating, preferably increasing, expression of a nucleic acid sequence encoding a PHDf-HD polypeptide is by introducing and expressing in a plant a nucleic acid sequence encoding a PHDf-HD polypeptide.

Any reference hereinafter to a “protein useful in the methods of the invention” is taken to mean a PHDf-HD polypeptide as defined herein. Any reference hereinafter to a “nucleic acid sequence useful in the methods of the invention” is taken to mean a nucleic acid sequence capable of encoding such a PHDf-HD polypeptide. The nucleic acid sequence to be introduced into a plant (and therefore useful in performing the methods of the invention) is any nucleic acid sequence encoding the type of polypeptide, which will now be described, hereafter also named “PHDf-HD nucleic acid sequence” or “PHDf-HD gene”.

A “PHDf-HD polypeptide” as defined herein refers to any polypeptide comprising: (i) a domain having at least 25%, 30%, 35%, 40%, 45%, 50%, 55%, 60%, 65%, 70%, 75%, 80%, 85%, 90%, 95%, 98%, 99% or more amino acid sequence identity to a leucine zipper/plant homeodomain finger (ZIP/PHDf) domain as represented by SEQ ID NO: 233; and (ii) a domain having at least 25%, 30%, 35%, 40%, 45%, 50%, 55%, 60%, 65%, 70%, 75%, 80%, 85%, 90%, 95%, 98%, 99% or more amino acid sequence identity to a homeodomain (HD) as represented by SEQ ID NO: 234.

Alternatively or additionally, a “PHDf-HD polypeptide” as defined herein refers to any polypeptide comprising: (i) a PHD domain as represented by PFAM00628; and (ii) an HD as represented by PFAM00046.

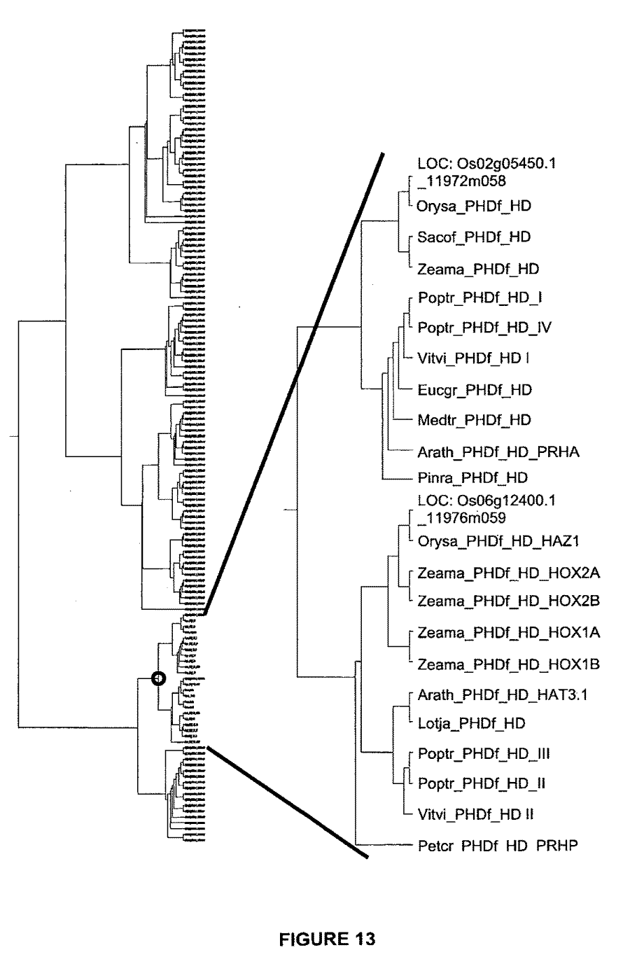

Alternatively or additionally, a “PHDf-HD polypeptide” as defined herein refers to any polypeptide sequence which when used in the construction of a HD phylogenetic tree, such as the one depicted in FIG. 13, clusters with the PHDf-HD group of polypeptides comprising the polypeptide sequence as represented by SEQ ID NO: 180, rather than with any other HD group.

Alternatively or additionally, a “PHDf-HD polypeptide” as defined herein refers to any polypeptide having in increasing order of preference at least 25%, 30%, 35%, 40%, 45%, 50%, 55%, 60%, 65%, 70%, 75%, 80%, 85%, 90%, 95%, 98%, 99% or more amino acid sequence identity to the PHDf-HD polypeptide as represented by SEQ ID NO: 180 or to any of the polypeptide sequences given in Table D1 herein.

The term “domain” and “motif” is defined in the “definitions” section herein. Specialist databases exist for the identification of domains, for example, SMART (Schultz et al. (1998) Proc. Natl. Acad. Sci, USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hula et al., Nucl. Acids. Res. 32:D134-D137, (2004), or Pfam (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment. Analysis of the polypeptide sequence of SEQ ID NO: 180 is presented below in Examples 30 and 32 herein. For example, a PHDf-HD polypeptide as represented by SEQ ID NO: 180 comprises a PHD finger with a Pfam entry PF00628, and a HD with Pfam entry PF00046.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or polypeptide sequence or over selected domains or conserved motif(s), using the programs mentioned above using the default parameters. Example 31 herein describes in Table D2 the percentage identity between the ZIP/PHDf as represented by SEQ ID NO: 233 and ZIP/PHDf of the PHDf-HD polypeptides listed in Table D1, and in Table D3 the percentage identity between the HD as represented by SEQ ID NO: 234 and the HD of the PHDf-HD polypeptides listed in Table D1 of Example 29.

Furthermore, amino acid sequences enriched in basic (Lys and Arg) or acidic (Glu and Asp) amino acids, respectively called basic and acidic stretches, may also readily be identified simply by eye inspection (FIG. 17). Alternatively primary amino acid composition (in %) may be calculated to determine if a polypeptide domain is rich in specific amino acids using software programs from the ExPASy server, in particular the ProtParam tool (Gasteiger E et al. (2003) ExPASy: the proteomics server for in-depth protein knowledge and analysis. Nucleic Acids Res 31:3784-3788). The composition of the protein of interest may then be compared to the average amino acid composition (in %) in the Swiss-Prot Protein Sequence data bank.

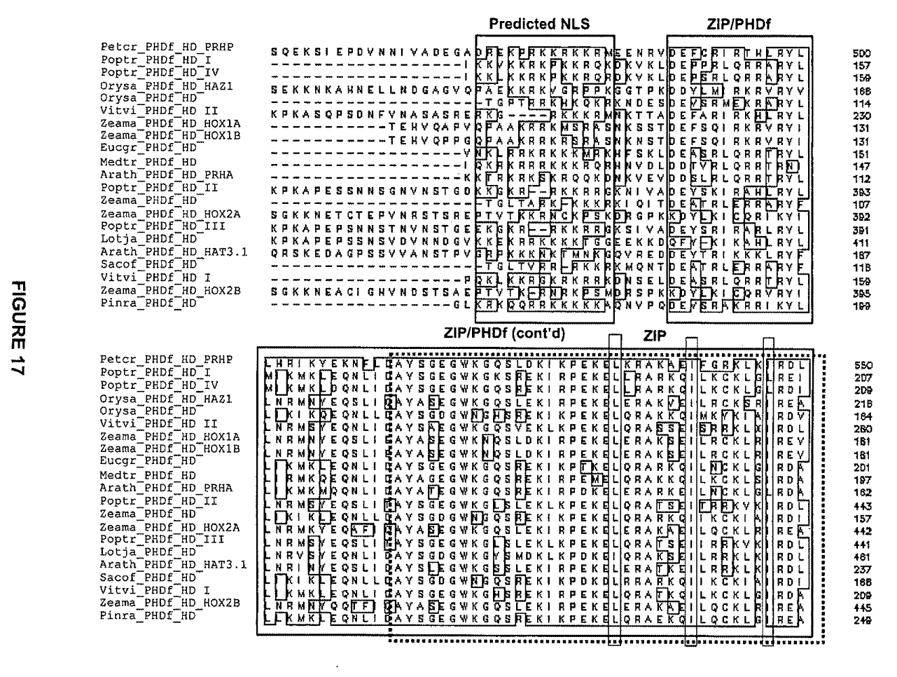

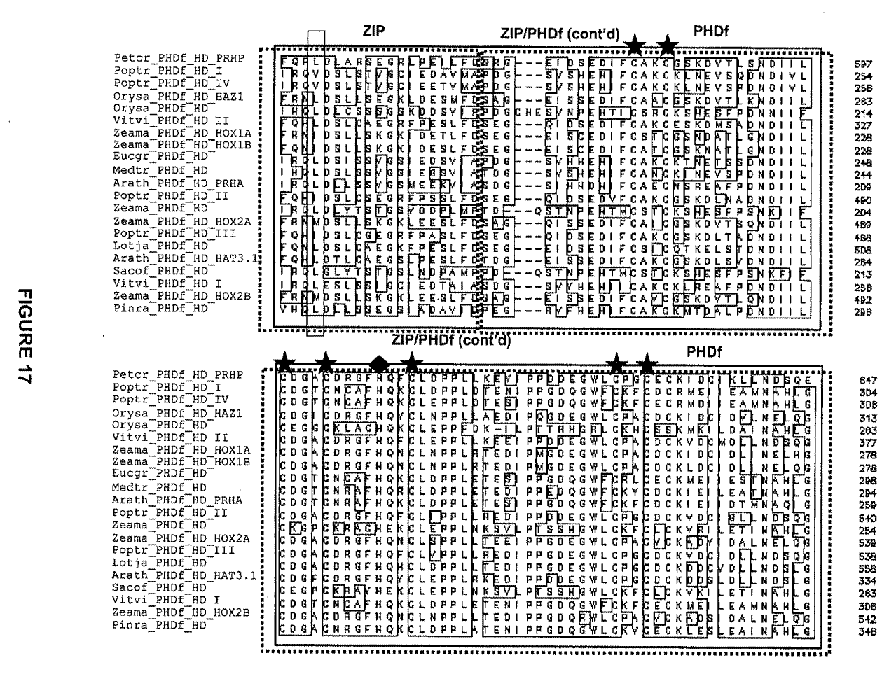

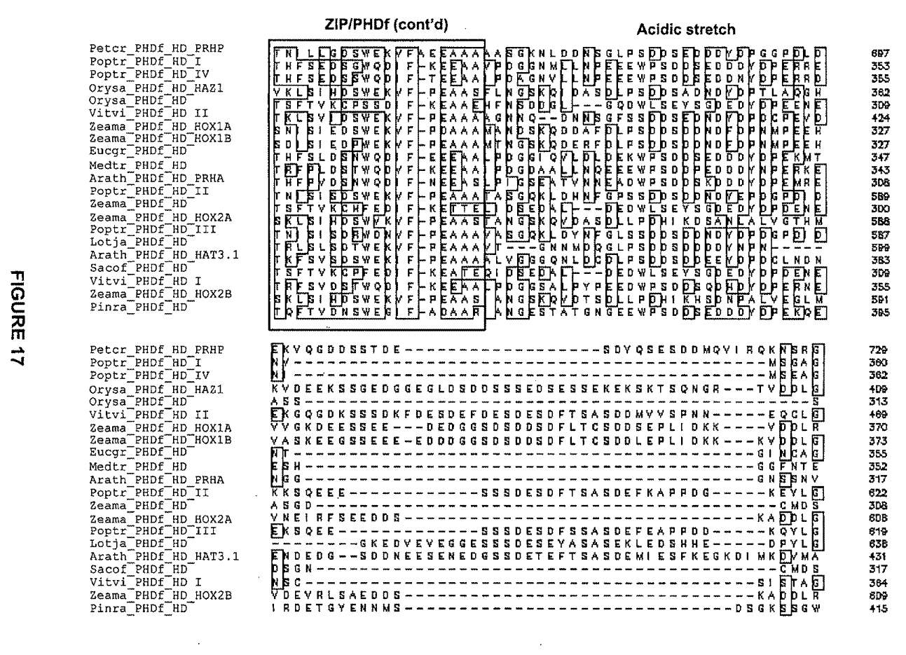

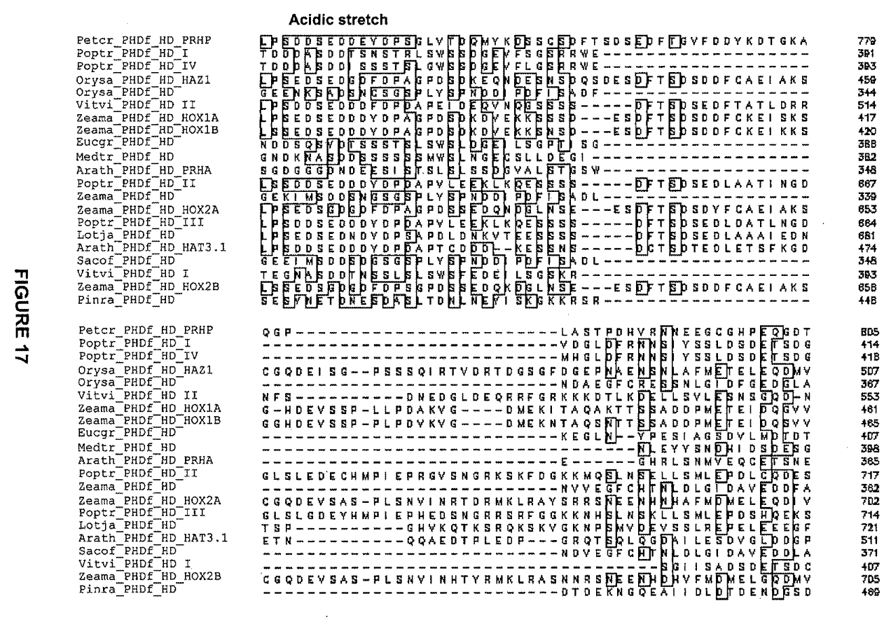

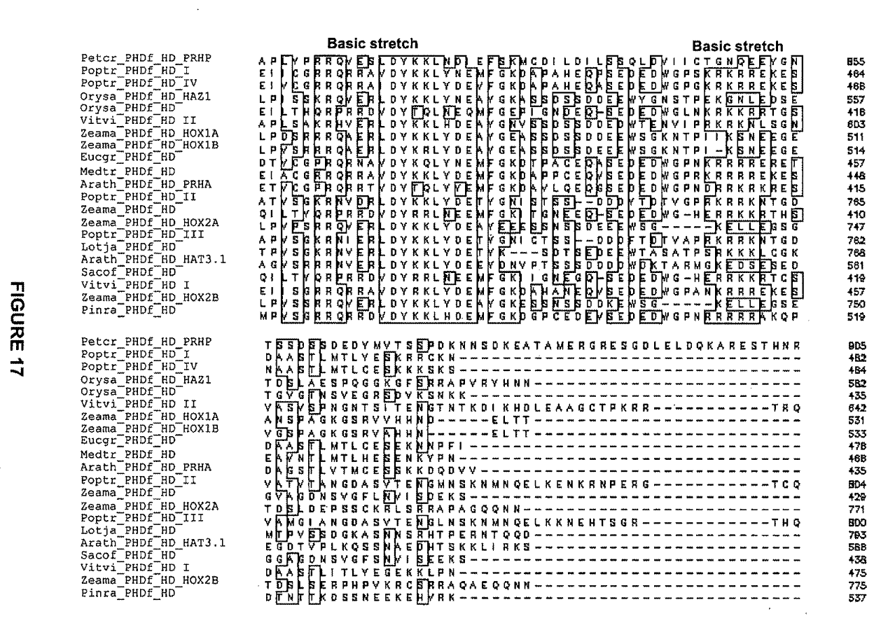

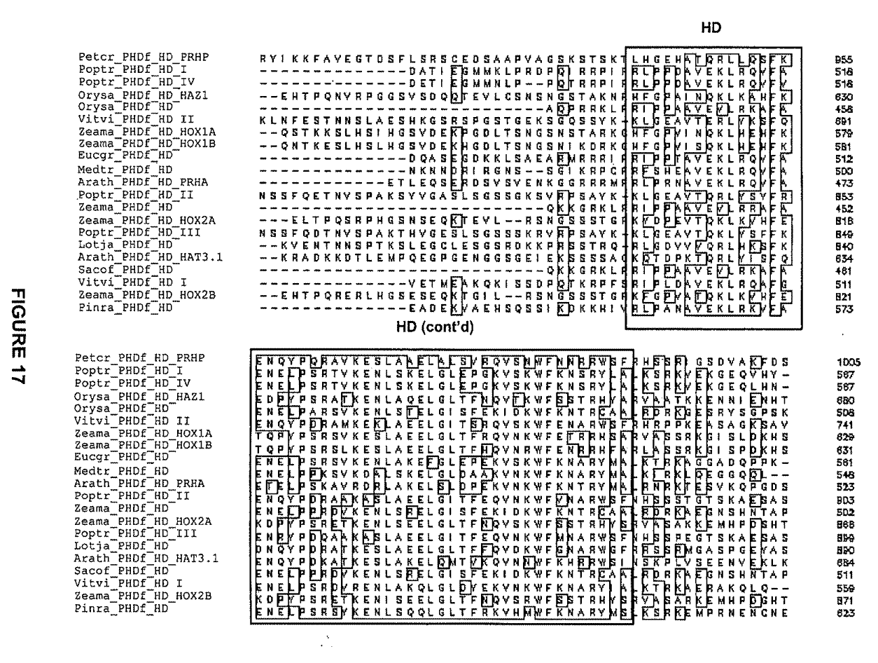

Coiled coils are important to identify for protein-protein interactions, such as oligomerization, either of identical proteins, of proteins of the same family, or of unrelated proteins. A PHDf-HD polypeptide presents at least one predicted coiled coil region. Recently much progress has been made in computational prediction of coiled coils from sequence data. Among algorithms well known to a person skilled in the art are available at the ExPASy Proteomics tools COILS, PAIRCOIL, PAIRCOIL2, MULTICOIL, or MARCOIL, hosted by the Swiss Institute for Bioinformatics. In Example 33 and FIG. 16, are shown respectively the numerical and graphical results of SEQ ID NO: 180 as produced by the COILS algorithm analysis. Two N-terminal predicted coiled coil domains are identified in a PHDf-HD polypeptide sequence as represented by SEQ ID NO: 180, with a strong probability. A C-terminal coiled coil is also predicted, with a lower probability.

The task of protein subcellular localisation prediction is important and well studied. Knowing a protein's localisation helps elucidate its function. Experimental methods for protein localization range from immunolocalization to tagging of proteins using green fluorescent protein (GFP). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP and others. The identification of subcellular localisation of the polypeptide of the invention is shown in Example 34. In particular SEQ ID NO: 180 of the present invention is assigned to the nuclear compartment of eucaryotic cells.

Furthermore, PHDf-HD polypeptides useful in the methods of the present invention (at least in their native form) typically, but not necessarily, have transcriptional regulatory activity and capacity to interact with other proteins. Therefore, PHDf-HD polypeptides with reduced transcriptional regulatory activity, without transcriptional regulatory activity, with reduced protein-protein interaction capacity, or with no protein-protein interaction capacity, may equally be useful in the methods of the present invention. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). To determine the DNA binding activity of PHDf-HD polypeptides, several assays are available, such as DNA binding gel-shift assays (or gel retardation assays; Korfhage et al. (1994) Plant C 6: 695-708), in vitro DNA binding assays (Schindler et al. (1993) Plant J 4(1): 137-150), or transcriptional activation of PHDf-HD polypeptides in yeast, animal and plant cells (Halbach et al. (2000) Nucleic Acid Res 28(18): 3542-3550). Specific DNA binding sequences can be determined using the random oligonucleotide selection technique (Viola & Gonzalez (May 26, 2007) Biochemistry).

The present invention is illustrated by transforming plants with the nucleic acid sequence represented by SEQ ID NO: 179, encoding the polypeptide sequence of SEQ ID NO: 180. However, performance of the invention is not restricted to these sequences; the methods of the invention may advantageously be performed using any nucleic acid sequence encoding PHDf-HD or PHDf-HD polypeptide as defined herein.

Examples of nucleic acid sequences encoding PHDf-HD polypeptides are given in Table D1 of Example 29 herein. Such nucleic acid sequences are useful in performing the methods of the invention. The polypeptide sequences given in Table D1 of Example 29 are example sequences of orthologues and paralogues of the PHDf-HD polypeptide represented by SEQ ID NO: 180, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table D1 of Example 29) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 179 or SEQ ID NO: 180, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

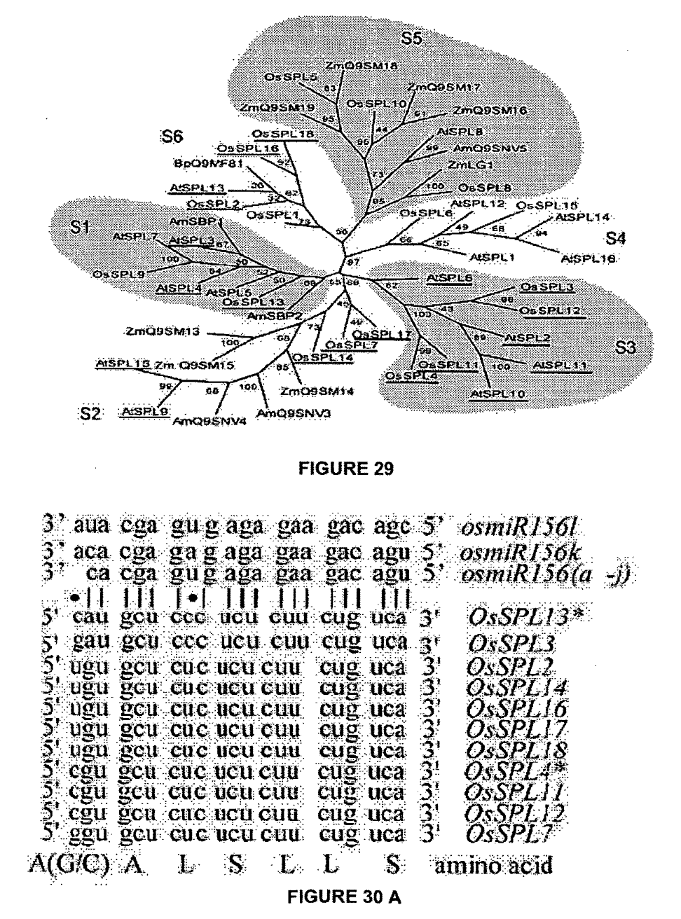

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues. Any sequence clustering within the group comprising SEQ ID NO: 180 (encircled in FIG. 29) would be considered to fall within the aforementioned definition of a PHDf-HD polypeptide, and would be considered suitable for use in the methods of the invention.

Nucleic acid variants may also be useful in practising the methods of the invention. Examples of such variants include nucleic acid sequences encoding homologues and derivatives of any one of the polypeptide sequences given in Table D1 of Example 29, the terms “homologue” and “derivative” being as defined herein. Also useful in the methods of the invention are nucleic acid sequences encoding homologues and derivatives of orthologues or paralogues of any one of the polypeptide sequences given in Table D1 of Example 29. Homologues and derivatives useful in the methods of the present invention have substantially the same biological and functional activity as the unmodified protein from which they are derived.

Further nucleic acid variants useful in practising the methods of the invention include portions of nucleic acid sequences encoding PHDf-HD polypeptides, nucleic acid sequences hybridising to nucleic acid sequences encoding PHDf-HD polypeptides, splice variants of nucleic acid sequences encoding PHDf-HD polypeptides, allelic variants of nucleic acid sequences encoding PHDf-HD polypeptides and variants of nucleic acid sequences encoding PHDf-HD polypeptides obtained by gene shuffling. The terms hybridising sequence, splice variant, allelic variant and gene shuffling are as described herein.

Nucleic acid sequences encoding PHDf-HD polypeptides need not be full-length nucleic acid sequences, since performance of the methods of the invention does not rely on the use of full-length nucleic acid sequences. According to the present invention, there is provided a method for enhancing yield-related traits, preferably enhancing seed yield-related traits, in plants, comprising introducing and expressing in a plant a portion of any one of the nucleic acid sequences given in Table D1 of Example 29, or a portion of a nucleic acid sequence encoding an orthologue, paralogue or homologue of any of the polypeptide sequences given in Table D1 of Example 29.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a PHDf-HD polypeptide as defined herein, and have substantially the same biological activity as the polypeptide sequences given in Table D1 of Example 29. Preferably, the portion is a portion of any one of the nucleic acid sequences given in Table D1 of Example 29, or is a portion of a nucleic acid sequence encoding an orthologue or paralogue of any one of the polypeptide sequences given in Table D1 of Example 29. Preferably the portion is, in increasing order of preference at least 600, 800, 1000, 1200, 1400, 1600, 1800, 2000, 2200, 2400, 2600, 2800 consecutive nucleotides in length, the consecutive nucleotides being of any one of the nucleic acid sequences given in Table D1 of Example 29, or of a nucleic acid sequence encoding an orthologue or paralogue of any one of the polypeptide sequences given in Table D1 of Example 29. Most preferably the portion is a portion of the nucleic acid sequence of SEQ ID NO: 179. Preferably, the portion encodes a polypeptide sequence which when used in the construction of a HID phylogenetic tree, such as the one depicted in FIG. 13, clusters with the group of PHDf-HD polypeptides comprising the polypeptide sequence represented by SEQ ID NO: 180 rather than with any other HD group.

Another nucleic acid sequence variant useful in the methods of the invention is a nucleic acid sequence capable of hybridising, under reduced stringency conditions, preferably under stringent conditions, with a nucleic acid sequence encoding a PHDf-HD polypeptide as defined herein, or with a portion as defined herein.

According to the present invention, there is provided a method for enhancing yield-related traits in plants, preferably enhancing seed yield-related traits, comprising introducing and expressing in a plant a nucleic acid sequence capable of hybridizing to any one of the nucleic acid sequences given in Table D1 of Example 29, or comprising introducing and expressing in a plant a nucleic acid sequence capable of hybridising to a nucleic acid sequence encoding an orthologue, paralogue or homologue of any of the nucleic acid sequences given in Table D1 of Example 29.

Hybridising sequences useful in the methods of the invention encode a PHDf-HD polypeptide as defined herein, and have substantially the same biological activity as the polypeptide sequences given in Table D1 of Example 29. Preferably, the hybridising sequence is capable of hybridising to any one of the nucleic acid sequences given in Table D1 of Example 29, or to a portion of any of these sequences, a portion being as defined above, or wherein the hybridising sequence is capable of hybridising to a nucleic acid sequence encoding an orthologue or paralogue of any one of the polypeptide sequences given in Table D1 of Example 29. Most preferably, the hybridising sequence is capable of hybridising to a nucleic acid sequence as represented by SEQ ID NO: 179 or to a portion thereof.

Preferably, the hybridising sequence encodes a polypeptide sequence which when used in the construction of a HD phylogenetic tree, such as the one depicted in FIG. 13, clusters with the group of PHDf-HD polypeptides comprising the polypeptide sequence represented by SEQ ID NO: 180 rather than with any other HO group.

Another nucleic acid sequence variant useful in the methods of the invention is a splice variant encoding a PHDf-HD polypeptide as defined hereinabove, a splice variant being as defined herein.

According to the present invention, there is provided a method for enhancing yield-related traits in plants, preferably enhancing seed yield-related traits, comprising introducing and expressing in a plant a splice variant of any one of the nucleic acid sequences given in Table D1 of Example 29, or a splice variant of a nucleic acid sequence encoding an orthologue, paralogue or homologue of any of the polypeptide sequences given in Table D1 of Example 29.

Preferred splice variants are splice variants of a nucleic acid sequence represented by SEQ ID NO: 179, or a splice variant of a nucleic acid sequence encoding an orthologue or paralogue of SEQ ID NO: 180. Preferably, the polypeptide sequence encoded by the splice variant, when used in the construction of a HD phylogenetic tree, such as the one depicted in FIG. 13, clusters with the group of PHDf-HD polypeptides comprising the polypeptide sequence represented by SEQ ID NO: 180 rather than with any other HD group.

Another nucleic acid sequence variant useful in performing the methods of the invention is an allelic variant of a nucleic acid sequence encoding a PHDf-HD polypeptide as defined hereinabove, an allelic variant being as defined herein.

According to the present invention, there is provided a method for enhancing yield-related traits in plants, preferably enhancing seed yield-related traits, comprising introducing and expressing in a plant an allelic variant of any one of the nucleic acid sequences given in Table D1 of Example 29, or comprising introducing and expressing in a plant an allelic variant of a nucleic acid sequence encoding an orthologue, paralogue or homologue of any of the polypeptide sequences given in Table D1 of Example 29.

The allelic variants useful in the methods of the present invention have substantially the same biological activity as the polypeptide of SEQ ID NO: 180 and any of the polypeptide sequences depicted in Table D1 of Example 29. Allelic variants exist in nature, and encompassed within the methods of the present invention is the use of these natural alleles. Preferably, the allelic variant is an allelic variant of SEQ ID NO: 179 or an allelic variant of a nucleic acid sequence encoding an orthologue or paralogue of SEQ ID NO: 180. Preferably, the polypeptide sequence encoded by the allelic variant, when used in the construction of a HD phylogenetic tree, such as the one depicted in FIG. 13, clusters with the PHDf-HD polypeptides comprising the polypeptide sequence represented by SEQ ID NO: 180 rather than with any other HD group.

Gene shuffling or directed evolution may also be used to generate variants of nucleic acid sequences encoding PHDf-HD polypeptides as defined above; the term “gene shuffling” being as defined herein.

According to the present invention, there is provided a method for enhancing yield-related traits in plants, preferably enhancing seed yield-related traits, comprising introducing and expressing in a plant a variant of any one of the nucleic acid sequences given in Table D1 of Example 29, or comprising introducing and expressing in a plant a variant of a nucleic acid sequence encoding an orthologue, paralogue or homologue of any of the polypeptide sequences given in Table D1 of Example 29, which variant nucleic acid sequence is obtained by gene shuffling.

Advantageously, the present invention provides hitherto unknown PHDf-HD nucleic acid and polypeptide sequences.

According to a further embodiment of the present invention, there is provided an isolated nucleic acid molecule comprising:
(i) a nucleic acid represented by SEQ ID NO: 242;
(ii) a nucleic acid or fragment thereof that is complementary to SEQ ID NO: 242;
(iii) a nucleic acid encoding an JMJC polypeptide having, in increasing order of preference, at least 70%, 75%, 80%, 85%, 90%, 95%, 96%, 97%, 98%, 99% or more sequence identity to the amino acid sequence given in SEQ ID NO: 243;
(iv) a nucleic acid capable of hybridizing under stringent conditions to any one of the nucleic acids given in (i), (ii) or (iii) above.

According to a further embodiment of the present invention, there is therefore provided an isolated polypeptide comprising:
(i) a nucleic acid represented by SEQ ID NO: 242;
(ii) a nucleic acid or fragment thereof that is complementary to SEQ ID NO: 242;
(iii) a nucleic acid encoding an JMJC polypeptide having, in increasing order of preference, at least 70%, 75%, 80%, 85%, 90%, 95%, 96%, 97%, 98%, 99% or more sequence identity to the amino acid sequence given in SEQ ID NO: 243;
(iv) a nucleic acid capable of hybridizing under stringent conditions to any one of the nucleic acids given in (i), (ii) or (iii) above.

Preferably, the polypeptide sequence encoded by the variant nucleic acid sequence obtained by gene shuffling, when used in the construction of a HD phylogenetic tree, such as the one depicted in FIG. 13, clusters with the group of PHDf-HD polypeptides comprising the polypeptide sequence represented by SEQ ID NO: 180 rather than with any other HD group.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding PHDf-HD polypeptides may be derived from any natural or artificial source. The nucleic acid sequence may be modified from its native form in composition and/or genomic environment through deliberate human manipulation. Preferably the PHDf-HD polypeptide-encoding nucleic acid sequence is from a plant, further preferably from a monocotyledonous plant, more preferably from the family Poaceae, most preferably the nucleic acid sequence is from Oryza sativa.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating, preferably increasing, expression in a plant of a nucleic acid sequence encoding a PHDf-HD polypeptide as defined herein.

Since the transgenic plants according to the present invention have enhanced yield-related traits, preferably enhanced seed yield-related traits, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced (early) vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and 1-90 (time taken for plants to reach 90% of their maximal size), amongst others. The growth rate as defined herein is not taken to mean earlier flowering.

According to a preferred feature of the present invention, performance of the methods of the invention gives plants having an increased growth rate relative to control plants. Therefore, according to the present invention, there is provided a method for increasing the growth rate of plants, which method comprises modulating, preferably increasing, expression in a plant of a nucleic acid sequence encoding a PHDf-HD polypeptide as defined herein.

Enhanced yield-related traits, preferably enhanced seed yield-related traits, occur whether the plant is under non-stress conditions or whether the plant is exposed to various stresses compared to control plants grown under comparable conditions. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes, and insects. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions having enhanced yield-related traits, preferably enhanced seed yield-related traits, relative to control plants grown under comparable conditions. Therefore, according to the present invention, there is provided a method for enhancing yield-related traits in plants grown under non-stress conditions or under mild drought conditions, which method comprises modulating, preferably increasing, expression in a plant of a nucleic acid sequence encoding a PHDf-HD polypeptide. Plants with optimal growth conditions, cultivated under non-stress conditions, typically yield in increasing order of preference at least 90%, 87%, 85%, 83%, 80%, 77% or 75% of the average production of such plant in a given environment. Average production may be calculated on harvest and/or season basis on a given location. Persons skilled in the art are aware of average yield productions of a crop.

Performance of the methods according to the present invention results in plants grown under abiotic stress conditions having enhanced yield-related traits, preferably enhanced seed yield-related traits, relative to control plants grown under comparable stress conditions. As reported in Wang at al (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani at al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, the exemplification of the present invention with drought stress should not be seen as a limitation to drought stress, but more as a screen to indicate the involvement of PHDf-HD polypeptides as defined above, in enhancing yield-related traits, preferably enhancing seed yield-related traits, relative to control plants grown in comparable stress conditions, in abiotic stresses in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl₂, CaCl₂, amongst others.

Performance of the methods of the invention gives plants having enhanced yield-related traits, preferably enhanced seed yield-related traits, under abiotic stress conditions relative to control plants grown in comparable stress conditions. Therefore, according to the present invention, there is provided a method for enhancing yield-related traits, preferably enhancing seed yield-related traits, in plants grown under abiotic stress conditions, which method comprises modulating, preferably increasing, expression in a plant of a nucleic acid sequence encoding a PHDf-HD polypeptide. According to one aspect of the invention, the abiotic stress is an osmotic stress, selected from one or more of the following: water stress, salt stress, oxidative stress and ionic stress.

Another example of abiotic environmental stress is the reduced availability of one or more nutrients that need to be assimilated by the plants for growth and development. Because of the strong influence of nutrition utilization efficiency on plant yield and product quality, a huge amount of fertilizer is poured onto fields to optimize plant growth and quality. Productivity of plants ordinarily is limited by three primary nutrients, phosphorous, potassium and nitrogen, which is usually the rate-limiting element in plant growth of these three. Therefore the major nutritional element required for plant growth is nitrogen (N). It is a constituent of numerous important compounds found in living cells, including amino acids, proteins (enzymes), nucleic acids, and chlorophyll. 1.5% to 2% of plant dry matter is nitrogen and approximately 16% of total plant protein. Thus, nitrogen availability is a major limiting factor for crop plant growth and production (Frink et al. (1999) Proc Natl Acad Sci USA 96(4): 1175-1180), and has as well a major impact on protein accumulation and amino acid composition. Therefore, of great interest are crop plants with enhanced yield-related traits, preferably enhanced seed yield-related traits, when grown under nitrogen-limiting conditions.

Performance of the methods of the invention gives plants grown under conditions of nutrient deficiency, particularly under conditions of nitrogen deficiency, enhanced yield-related traits, preferably enhanced seed yield-related traits, relative to control plants grown under comparable conditions. Therefore, according to the present invention, there is provided a method for enhancing yield-related traits, preferably enhancing seed yield-related traits, in plants grown under conditions of nutrient deficiency, which method comprises modulating, preferably increasing, expression in a plant of a nucleic acid sequence encoding a PHDf-HD polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) or cells thereof obtainable by the methods according to the present invention. The plants or parts thereof or cells thereof comprise a nucleic acid transgene encoding a PHDf-HD polypeptide as defined above.

The invention also provides genetic constructs and vectors to facilitate introduction and/or modulated, preferably increased, expression in plants of nucleic acid sequences encoding PHDf-HD polypeptides. The gene constructs may be inserted into vectors, which may be commercially available, suitable for transforming into plants and for expression of the gene of interest in the transformed cells. The invention also provides use of a gene construct as defined herein in the methods of the invention.

More specifically, the present invention provides a construct comprising:
(a) a nucleic acid sequence encoding a PHDf-HD polypeptide as defined above;
(b) one or more control sequences capable of modulating, preferably increasing, expression of the nucleic acid sequence of (a); and optionally
(c) a transcription termination sequence.

Preferably, the nucleic acid sequence encoding a PHDf-HD polypeptide is as defined above. The term “control sequence” and “termination sequence” are as defined herein.

Preferably, one of the control sequences of a construct is a constitutive promoter isolated from a plant genome. An example of a plant constitutive promoter is a GOS2 promoter, preferably a rice GOS2 promoter, more preferably a GOS2 promoter as represented by SEQ ID NO: 235.

Plants are transformed with a vector comprising any of the nucleic acid sequences described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to modulate, preferably increase, expression of the nucleic acid sequence. A constitutive promoter is particularly useful in the methods, preferably a constitutive promoter isolated from a plant genome. The plant constitutive promoter drives expression of a coding sequence at a level that is in all instances below that obtained under the control of a 35S CaMV promoter.

Other organ-specific promoters, for example for preferred expression in leaves, stems, tubers, meristems, seeds (embryo and/or endosperm), are useful in performing the methods of the invention. See the “Definitions” section herein for definitions of the various promoter types.

It should be clear that the applicability of the present invention is not restricted to the PHDf-HD polypeptide-encoding nucleic acid sequence represented by SEQ ID NO: 179, nor is the applicability of the invention restricted to expression of a PHDf-HD polypeptide-encoding nucleic acid sequence when driven by a constitutive promoter.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1

For the detection of the successful transfer of the nucleic acid sequences as used in the methods of the invention and/or selection of transgenic plants comprising these nucleic acid sequences, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits, preferably enhanced seed yield-related traits, relative to control plants, comprising introduction and expression in a plant of any nucleic acid sequence encoding a PHDf-HD polypeptide as defined hereinabove.

More specifically, the present invention provides a method for the production of transgenic plants having enhanced yield-related traits, preferably enhanced seed yield-related traits, relative to control plants, which method comprises:
(i) introducing and expressing in a plant, plant part, or plant cell a nucleic acid sequence encoding a PHDf-HD polypeptide, under the control of plant constitutive promoter; and
(ii) cultivating the plant cell under conditions promoting plant growth and development.

The nucleic acid sequence of (i) may be any of the nucleic acid sequences capable of encoding a PHDf-HD polypeptide as defined herein.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hagen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a PHDf-HD polypeptide as defined hereinabove, operably linked to a plant constitutive promoter. Preferred host cells according to the invention are plant cells. Host plants for the nucleic acid sequences or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants, which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant comprising an isolated nucleic acid sequence encoding a PHDf-HD (as defined hereinabove) operably linked to a plant constitutive promoter, such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acid sequences or genes, or gene products, are well documented in the art and examples are provided in the definitions section.

As mentioned above, a preferred method for modulating, preferably increasing, expression of a nucleic acid sequence encoding a PHDf-HD polypeptide is by introducing and expressing in a plant a nucleic acid sequence encoding a PHDf-HD polypeptide; however the effects of performing the method, i.e. enhancing yield-related traits, may also be achieved using other well known techniques, including but not limited to T-DNA activation tagging, TILLING, homologous recombination. A description of these techniques is provided in the definitions section.

The present invention also encompasses use of nucleic acid sequences encoding PHDf-HD polypeptides as described herein and use of these PHDf-HD polypeptides in enhancing any of the aforementioned yield-related traits, preferably seed yield-related traits, in plants.

Nucleic acid sequences encoding PHDf-HD polypeptide described herein, or the PHDf-HD polypeptides themselves, may find use in breeding programmes in which a DNA marker is identified that may be genetically linked to a PHDf-HD polypeptide-encoding gene. The genes/nucleic acid sequences, or the PHDf-HD polypeptides themselves may be used to define a molecular marker. This DNA or protein marker may then be used in breeding programmes to select plants having enhanced yield-related traits, preferably enhanced seed yield-related traits, as defined hereinabove in the methods of the invention.

Allelic variants of a gene/nucleic acid sequence encoding a PHDf-HD polypeptide may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give enhanced yield-related traits. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acid sequences encoding PHDf-HD polypeptides may also be used as probes for genetically and physically mapping the genes that they are a part of, and as markers for traits linked to those genes. Such information may be useful in plant breeding in order to develop lines with desired phenotypes. Such use of nucleic acid sequences encoding a PHDf-HD polypeptide requires only a nucleic acid sequence of at least 15 nucleotides in length. The nucleic acid sequences encoding a PHDf-HD polypeptide may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acid sequences encoding a PHDf-HD polypeptide. The resulting banding patterns may then be subjected to genetic analyses using computer programs such as MapMaker (Lander et al. (1987) Genomics 1: 174-181) in order to construct a genetic map. In addition, the nucleic acid sequences may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid sequence encoding a PHDf-HD polypeptide in the genetic map previously obtained using this population (Botstein et al. (1980) Am. J. Hum. Genet. 32: 314-331).

The production and use of plant gene-derived probes for use in genetic mapping is described in Bernatzky and Tanksley (1986) Plant Mal. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acid sequences. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic acid sequence Res. 18:3671), Radiation Hybrid Mapping (Walter et al. (1997) Nat. Genet. 7:22-28) and Happy Mapping (Dear and Cook (1989) Nucleic acid sequence Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, preferably enhanced seed yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### V. bHLH11-Like (Basic Helix-Loop-Helix 11)

Surprisingly, it has now been found that modulating expression in a plant of a nucleic acid encoding a bHLH11-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding a bHLH11-like polypeptide.

A preferred method for modulating (preferably, increasing) expression of a nucleic acid encoding a bHLH11-like polypeptide is by introducing and expressing in a plant a nucleic acid encoding a bHLH11-like polypeptide.

Any reference hereinafter to a “protein useful in the methods of the invention” is taken to mean a bHLH11-like polypeptide as defined herein. Any reference hereinafter to a “nucleic acid useful in the methods of the invention” is taken to mean a nucleic acid capable of encoding such a bHLH11-like polypeptide. The nucleic acid to be introduced into a plant (and therefore useful in performing the methods of the invention) is any nucleic acid encoding the type of protein which will now be described, hereafter also named “bHLH11-like nucleic acid” or “bHLH11-like gene”.

A “bHLH11-like polypeptide” as defined herein refers to any polypeptide comprising a basic domain followed by a HLH domain (HMMPFam PF00010, ProfileScan PS50888, SMART SM00353) thereby forming a basic helix-loop-helix domain (bHLH) (Interpro IPR001092). Preferably, the bHLH11-like polypeptide comprises at least one, preferably two, more preferably three, most preferably four or more of the following motifs:

### Motif 1 (SEQ ID NO: 246): (E/D)(D/S/E)(F/M)(L/F)(D/E/Q/L)(Q/H/E)

### Motif 2 (SEQ ID NO: 247): RA(R/I/Q)RG(Q/H)ATDPHSIAER

### Motif 3 (SEQ ID NO: 248): (M/IN/L)(K/R)(A/S/Q/D/N)LQ(E/DA)LVP

### Motif 4 (SEQ ID NO: 249): (M/I)(UI)DEI(IN/L)(D/E/G)Y(V/UI)(K/R)FL(Q/R)LQ(V/I)K

### Motif 5 (SEQ ID NO: 250): (V/I)LSMSR(UV)G

### Motif 6 (SEQ ID NO: 251): V(AN/L/I)(K/R)(L/M)(M/L)(E/D)(E/D/S/K/T)(D/N/S)(MN/I)(G/T/I)XAMQ(Y/L/F)L

wherein X can be any amino acid, but preferably one of S, T, A, M, K, N

### Motif 7 (SEQ ID NO: 252): (M/V)(P/S)(I/V)(S/T/A)LA

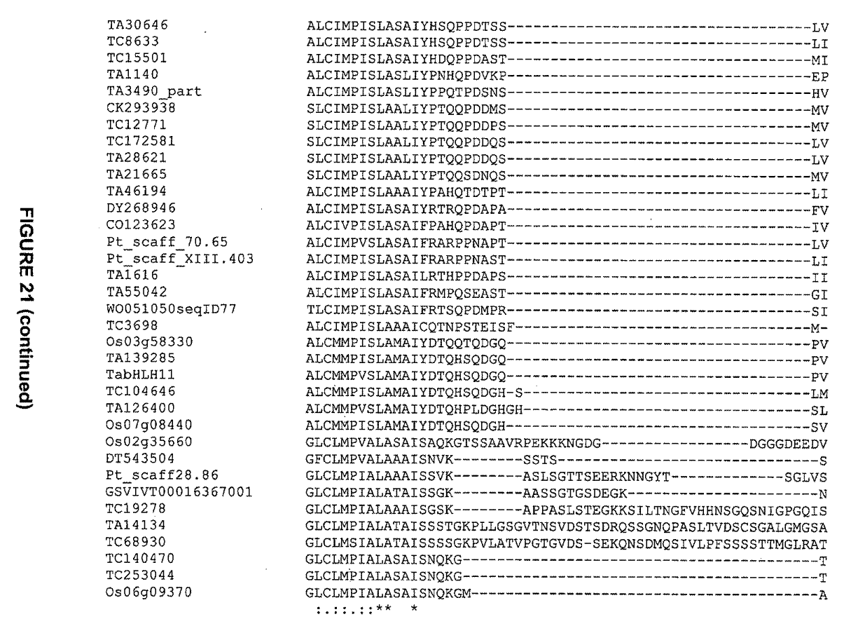

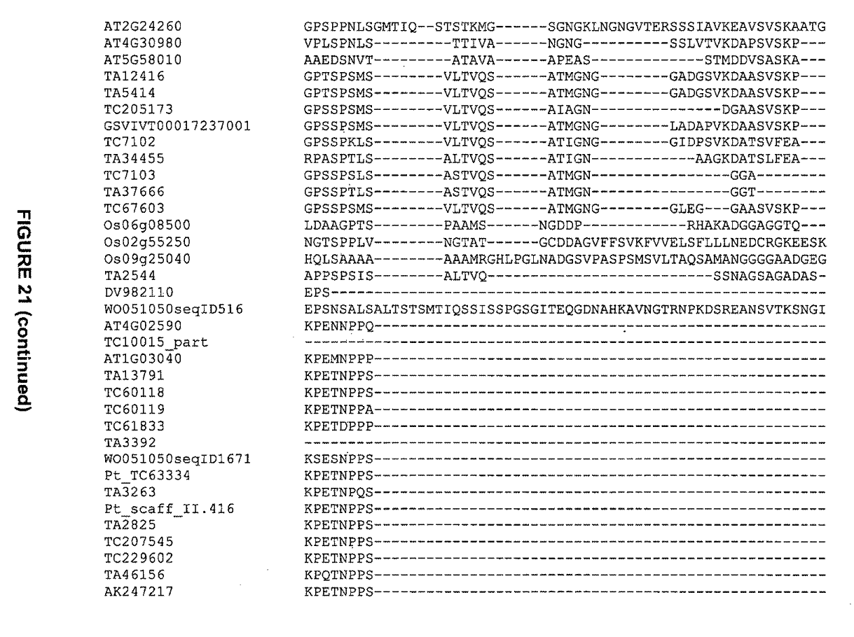

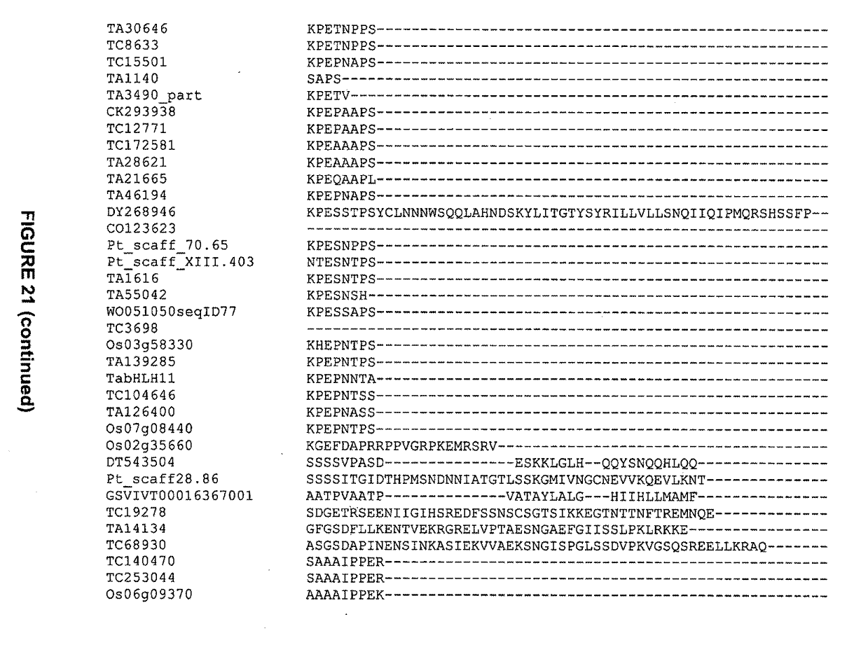

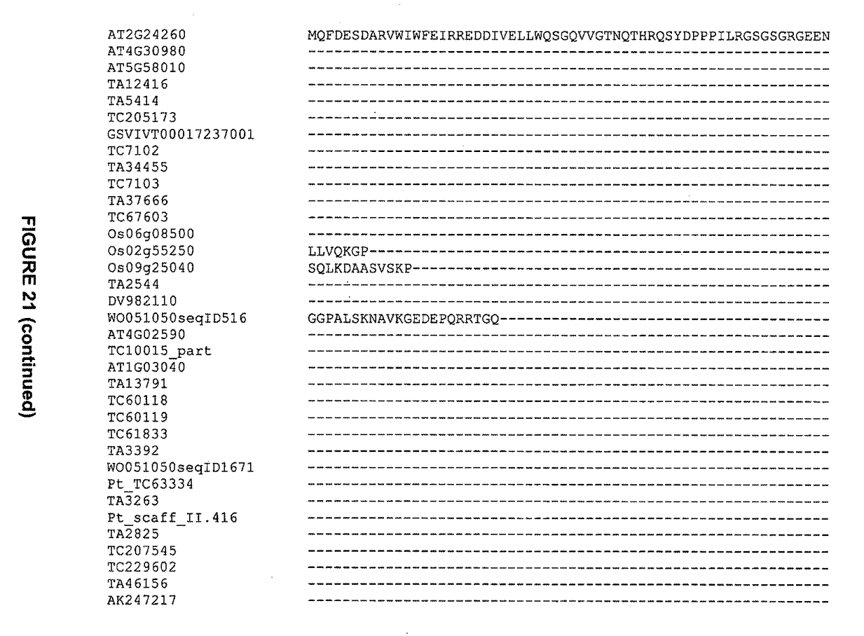

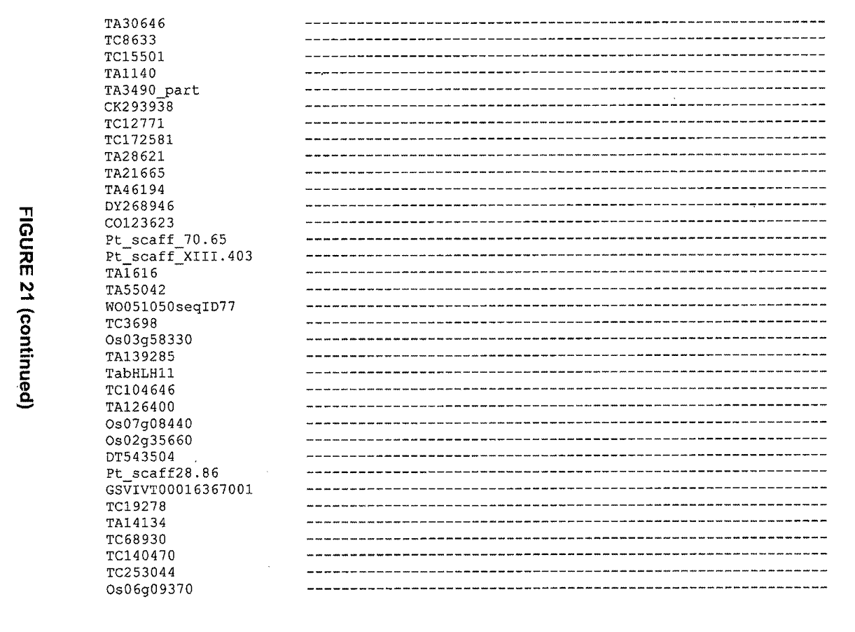

Alternatively, the homologue of a bHLH11-like protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 245, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered. The sequence conservation is much higher in the region of the bHLH domain (see Table E3 in Example 45 and FIG. 21). Therefore the bHLH domain is a good criterion for the defining the group of bHLH11-like proteins. Preferably, the bHLH11-like polypeptide comprises the sequence of Motif 8 (SEQ ID NO: 253): SIAERLRRERIAERMRALQELVPNTNKTDRAVMLDEILDYVKFLRLQVKVL,

or a sequence that has, in increasing order of preference, at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 253. The HLH domain as determined by SMART spans residue 132 to 181 in SEQ ID NO: 245 and is comprised in Motif 8.

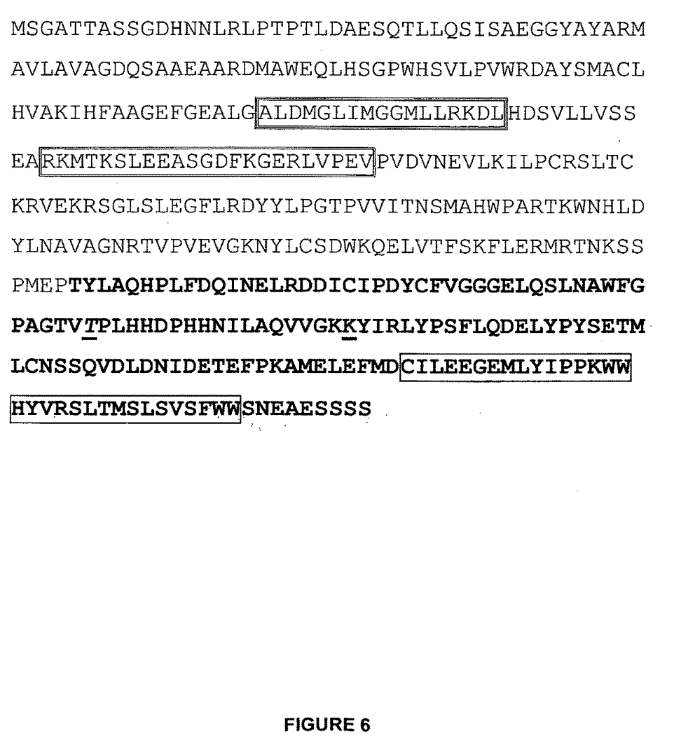

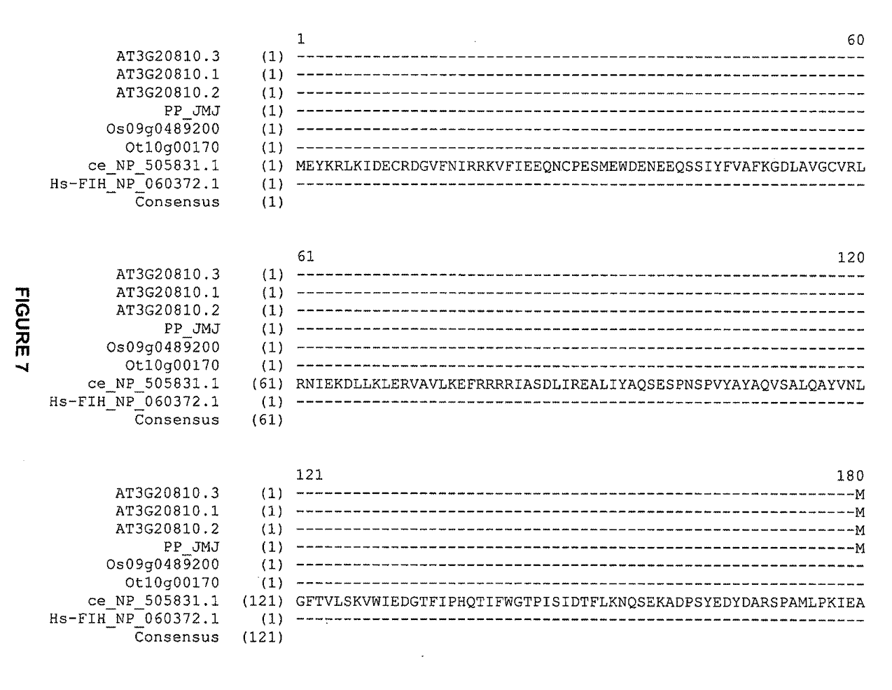

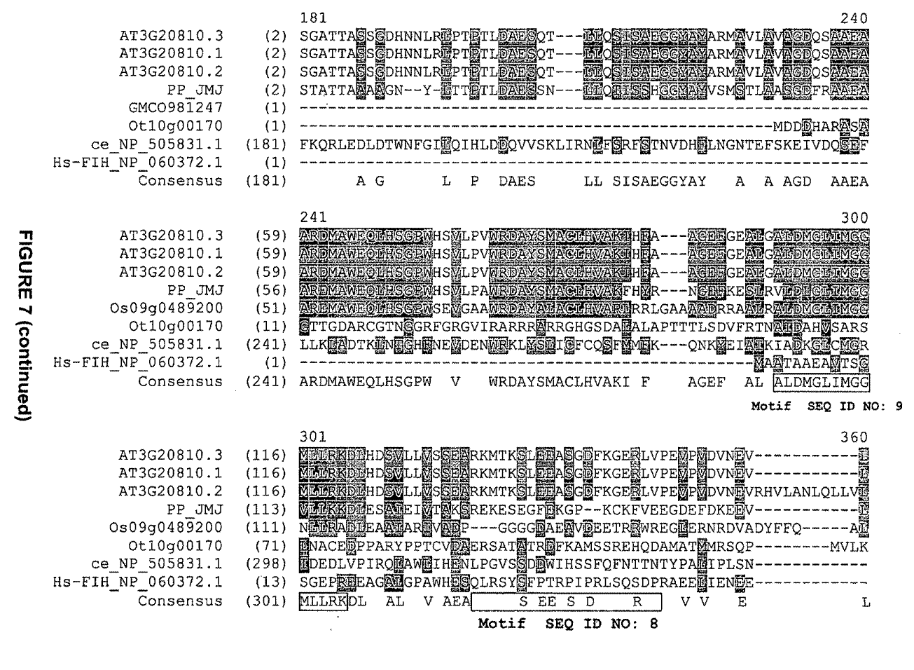

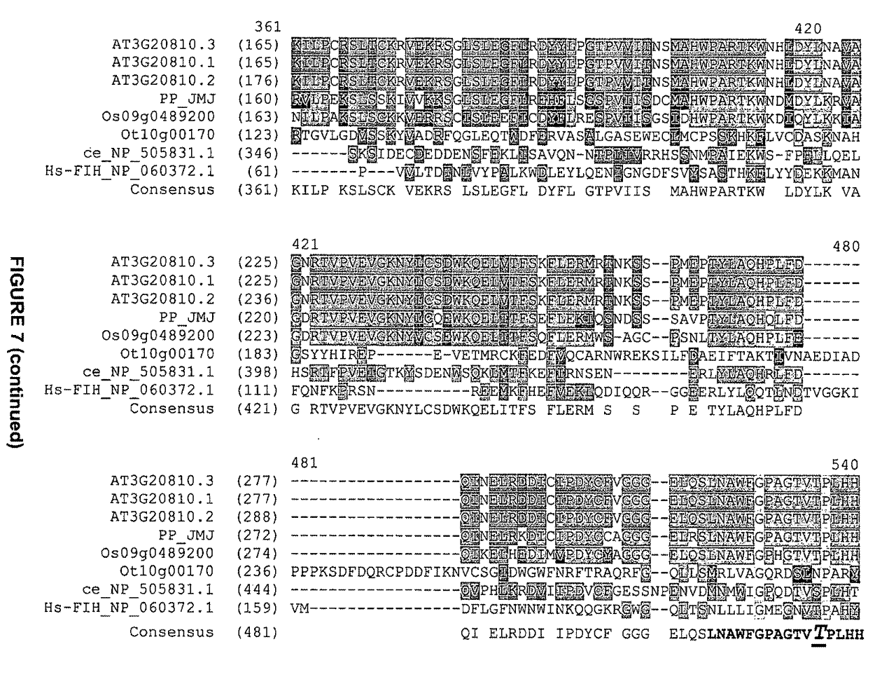

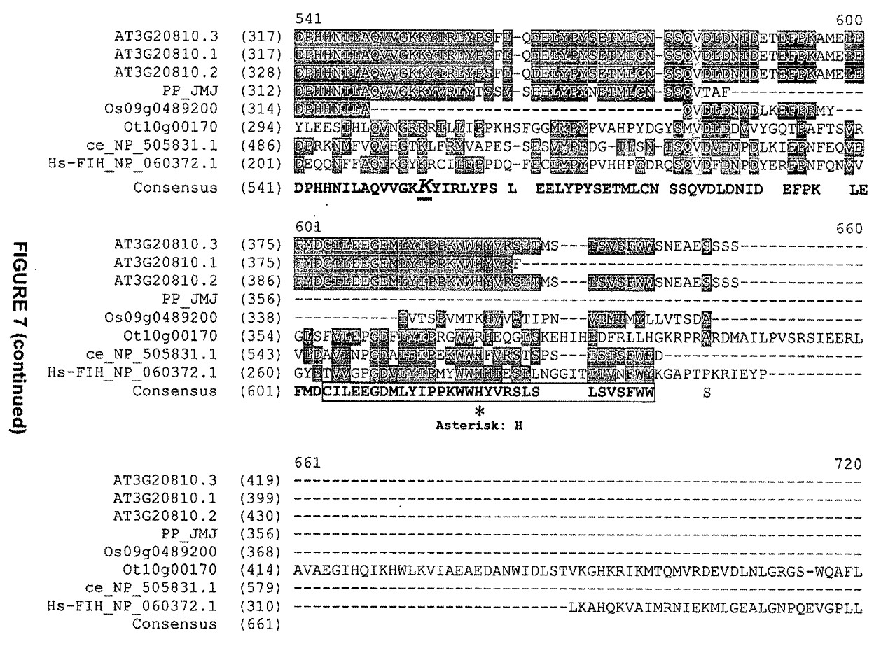

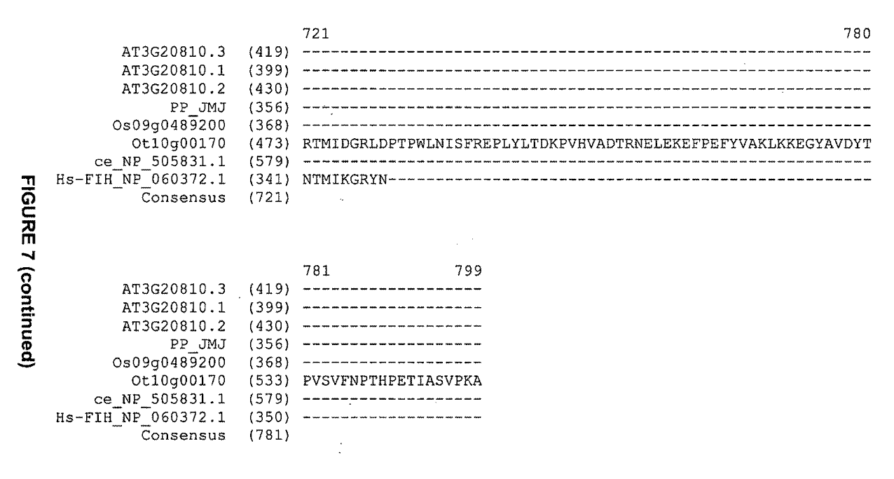

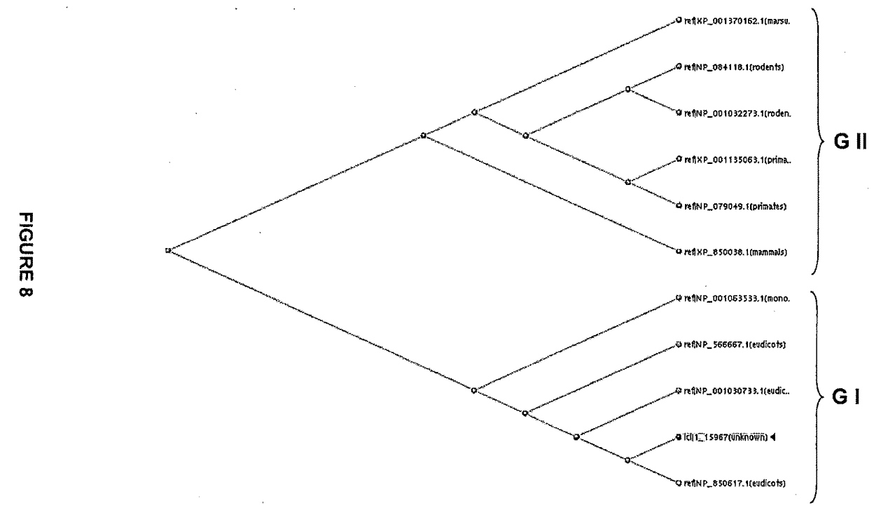

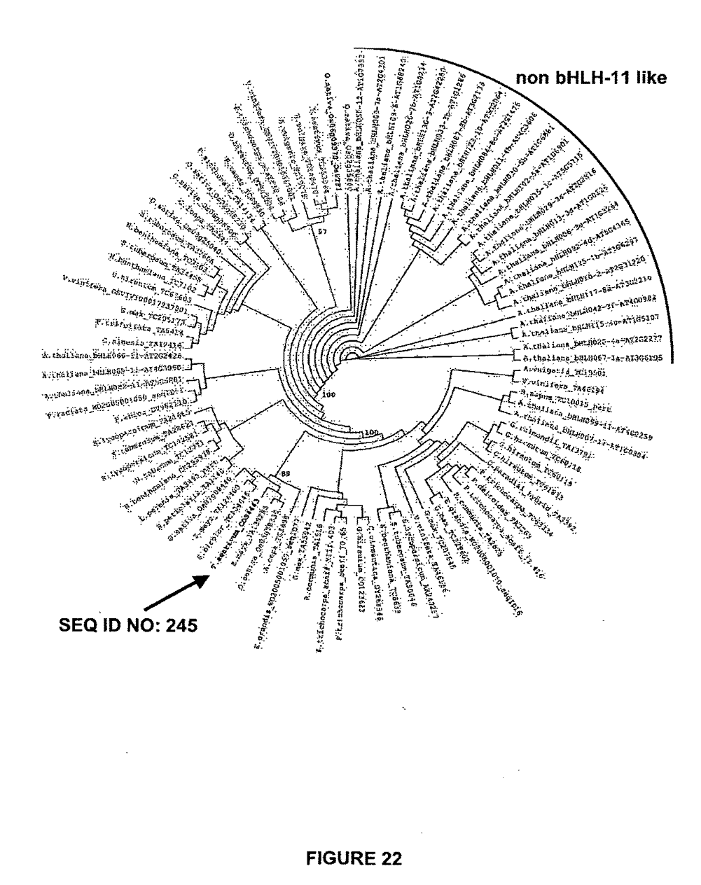

Preferably, the polypeptide sequence which when used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

Furthermore, bHLH11-like polypeptides (at least in their native form) typically have DNA binding activity. Tools and techniques for measuring DNA binding activity are well known in the art. In addition, as shown in the present invention, a bHLH11-like protein, such as SEQ ID NO: 245, when overexpressed in rice, gives plants having enhanced yield-related traits, in particular increased fill rate. Further details are provided in Examples section.

The present invention is illustrated by transforming plants with the nucleic acid sequence represented by SEQ ID NO: 244, encoding the polypeptide sequence of SEQ ID NO: 245. However, performance of the invention is not restricted to these sequences; the methods of the invention may advantageously be performed using any bHLH11-like-encoding nucleic acid or bHLH11-like polypeptide as defined herein.

Examples of nucleic acids encoding bHLH11-like polypeptides are given in Table E1 of Example 43 herein. Such nucleic acids are useful in performing the methods of the invention. The amino acid sequences given in Table E1 of Example 43 are example sequences of orthologues and paralogues of the bHLH11-like polypeptide represented by SEQ ID NO: 245, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table E1 of Example 43) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 244 or SEQ ID NO: 245, the second BLAST would therefore be against Triticum aestivum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table E1 of Example 43, the terms “homologue” and “derivative” being as defined herein. Also useful in the methods of the invention are nucleic acids encoding homologues and derivatives of orthologues or paralogues of any one of the amino acid sequences given in Table E1 of Example 43. Homologues and derivatives useful in the methods of the present invention have substantially the same biological and functional activity as the unmodified protein from which they are derived.

Further nucleic acid variants useful in practising the methods of the invention include portions of nucleic acids encoding bHLH11-like polypeptides, nucleic acids hybridising to nucleic acids encoding bHLH11-like polypeptides, splice variants of nucleic acids encoding bHLH11-like polypeptides, allelic variants of nucleic acids encoding bHLH11-like polypeptides and variants of nucleic acids encoding bHLH11-like polypeptides obtained by gene shuffling. The terms hybridising sequence, splice variant, allelic variant and gene shuffling are as described herein.

Nucleic acids encoding bHLH11-like polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table E1 of Example 43, or a portion of a nucleic acid encoding an orthologue, paralogue or homologue of any of the amino acid sequences given in Table E1 of Example 43.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a bHLH11-like polypeptide as defined herein, and have substantially the same biological activity as the amino acid sequences given in Table E1 of Example 43. Preferably, the portion is a portion of any one of the nucleic acids given in Table E1 of Example 43, or is a portion of a nucleic acid encoding an orthologue or paralogue of any one of the amino acid sequences given in Table E1 of Example 43. Preferably the portion is at least 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500 consecutive nucleotides in length, the consecutive nucleotides being of any one of the nucleic acid sequences given in Table E1 of Example 43, or of a nucleic acid encoding an orthologue or paralogue of any one of the amino acid sequences given in Table E1 of Example 43. Most preferably the portion is a portion of the nucleic acid of SEQ ID NO: 244. Preferably, the portion encodes a fragment of an amino acid sequence which, when used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

Another nucleic acid variant useful in the methods of the invention is a nucleic acid capable of hybridising, under reduced stringency conditions, preferably under stringent conditions, with a nucleic acid encoding a bHLH11-like polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table E1 of Example 43, or comprising introducing and expressing in a plant a nucleic acid capable of hybridising to a nucleic acid encoding an orthologue, paralogue or homologue of any of the nucleic acid sequences given in Table E1 of Example 43.

Hybridising sequences useful in the methods of the invention encode a bHLH11-like polypeptide as defined herein, having substantially the same biological activity as the amino acid sequences given in Table E1 of Example 43. Preferably, the hybridising sequence is capable of hybridising to the complement of any one of the nucleic acids given in Table E1 of Example 43, or to a portion of any of these sequences, a portion being as defined above, or the hybridising sequence is capable of hybridising to the complement of a nucleic acid encoding an orthologue or paralogue of any one of the amino acid sequences given in Table E1 of Example 43. Most preferably, the hybridising sequence is capable of hybridising to the complement of a nucleic acid as represented by SEQ ID NO: 244 or to a portion thereof.

Preferably, the hybridising sequence encodes a polypeptide with an amino acid sequence which, when full-length and used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

Another nucleic acid variant useful in the methods of the invention is a splice variant encoding a bHLH11-like polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table E1 of Example 43, or a splice variant of a nucleic acid encoding an orthologue, paralogue or homologue of any of the amino acid sequences given in Table E1 of Example 43.

Preferred splice variants are splice variants of a nucleic acid represented by SEQ ID NO: 244, or a splice variant of a nucleic acid encoding an orthologue or paralogue of SEQ ID NO: 245. Preferably, the amino acid sequence encoded by the splice variant, when used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

Another nucleic acid variant useful in performing the methods of the invention is an allelic variant of a nucleic acid encoding a bHLH11-like polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table E1 of Example 43, or comprising introducing and expressing in a plant an allelic variant of a nucleic acid encoding an orthologue, paralogue or homologue of any of the amino acid sequences given in Table E1 of Example 43.

The polypeptides encoded by allelic variants useful in the methods of the present invention have substantially the same biological activity as the bHLH11-like polypeptide of SEQ ID NO: 244 and any of the amino acids depicted in Table E1 of Example 43. Allelic variants exist in nature, and encompassed within the methods of the present invention is the use of these natural alleles. Preferably, the allelic variant is an allelic variant of SEQ ID NO: 244 or an allelic variant of a nucleic acid encoding an orthologue or paralogue of SEQ ID NO: 245. Preferably, the amino acid sequence encoded by the allelic variant, when used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

Gene shuffling or directed evolution may also be used to generate variants of nucleic acids encoding bHLH11-like polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table E1 of Example 43, or comprising introducing and expressing in a plant a variant of a nucleic acid encoding an orthologue, paralogue or homologue of any of the amino acid sequences given in Table E1 of Example 43, which variant nucleic acid is obtained by gene shuffling.

Preferably, the amino acid sequence encoded by the variant nucleic acid obtained by gene shuffling, when used in the construction of a phylogenetic tree, such as the one depicted in FIG. 22, clusters within the group of bHLH11-like proteins, rather than with other bHLH proteins. Similarly, the bHLH11-like protein of choice will cluster within subgroup C when a tree is constructed according to FIG. 6 in Li et al. (2006), rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding bHLH11-like polypeptides may be derived from any natural or artificial source. The nucleic acid may be modified from its native form in composition and/or genomic environment through deliberate human manipulation. Preferably the bHLH11-like polypeptide-encoding nucleic acid is from a plant, further preferably from a monocotyledonous plant, more preferably from the family Poaceae, most preferably the nucleic acid is from Triticum aestivurn.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression in a plant of a nucleic acid encoding a bHLH11-like polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and 1-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a bHLH11-like polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%; 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a bHLH11-like polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid encoding a bHLH11-like polypeptide, provided that the nutrient deficiency is not a phosphate deficiency. Nutrient deficiency may result from a lack of nutrients such as nitrogen, phosphates and other phosphorous-containing compounds, potassium, calcium, cadmium, magnesium, manganese, iron and boron, amongst others. However, the term “nutrient deficiency” as used in the context of the present invention does not encompass a deficiency in phosphate.

The present invention encompasses plants or parts thereof (including seeds) obtainable by the methods according to the present invention. The plants or parts thereof comprise a nucleic acid transgene encoding a bHLH11-like polypeptide as defined above.

The invention also provides genetic constructs and vectors to facilitate introduction and/or expression in plants of nucleic acids encoding bHLH11-like polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a bHLH11-like polypeptide as defined above;
(b) one or more control sequences capable of driving expression of the nucleic acid sequence of (a); and optionally
(c) a transcription termination sequence.

Preferably, the nucleic acid encoding a bHLH11-like polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence, but preferably the promoter is of plant origin. A constitutive promoter is particularly useful in the methods. Preferably the constitutive promoter is also a ubiquitous promoter of medium strength. See the “Definitions” section herein for definitions of the various promoter types.

It should be clear that the applicability of the present invention is not restricted to the bHLH11-like polypeptide-encoding nucleic acid represented by SEQ ID NO: 1, nor is the applicability of the invention restricted to expression of a bHLH11-like polypeptide-encoding nucleic acid when driven by a constitutive promoter.

The constitutive promoter is preferably a medium strength promoter, such as a GOS2 promoter, preferably the promoter is a GOS2 promoter from rice. Further preferably the constitutive promoter is represented by a nucleic acid sequence substantially similar to SEQ ID NO: 256, most preferably the constitutive promoter is as represented by SEQ ID NO: 256. See the “Definitions” section herein for further examples of constitutive promoters.

Optionally, one or more terminator sequences may be used in the construct introduced into a plant. Preferably, the construct comprises an expression cassette comprising the GOS2 promoter substantially similar to SEQ ID NO: 256 and the nucleic acid encoding the bHLH11-like polypeptide.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a bHLH11-like polypeptide as defined hereinabove.

More specifically, the present invention provides a method for the production of transgenic plants having increased enhanced yield-related traits, particularly increased (seed) yield, which method comprises:
(i) introducing and expressing in a plant or plant cell a bHLH11-like polypeptide-encoding nucleic acid; and
(ii) cultivating the plant cell under conditions promoting plant growth and development.

The nucleic acid of (i) may be any of the nucleic acids capable of encoding a bHLH11-like polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a bHLH11-like polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs, which harvestable parts comprise a recombinant nucleic acid encoding a bHLH11-like polypeptide. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a bHLH11-like polypeptide is by introducing and expressing in a plant a nucleic acid encoding a bHLH11-like polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding bHLH11-like polypeptides as described herein and use of these bHLH11-like polypeptides in enhancing any of the aforementioned yield-related traits in plants.

Nucleic acids encoding bHLH11-like polypeptide described herein, or the bHLH11-like polypeptides themselves, may find use in breeding programmes in which a DNA marker is identified which may be genetically linked to a bHLH11-like polypeptide-encoding gene. The nucleic acids/genes, or the bHLH11-like polypeptides themselves may be used to define a molecular marker. This DNA or protein marker may then be used in breeding programmes to select plants having enhanced yield-related traits as defined hereinabove in the methods of the invention.

Allelic variants of a bHLH11-like polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding bHLH11-like polypeptides may also be used as probes for genetically and physically mapping the genes that they are a part of, and as markers for traits linked to those genes. Such information may be useful in plant breeding in order to develop lines with desired phenotypes. Such use of bHLH11-like polypeptide-encoding nucleic acids requires only a nucleic acid sequence of at least 15 nucleotides in length. The bHLH11-like polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the bHLH11-like-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the bHLH11-like polypeptide-encoding nucleic acid in the genetic map previously obtained using this population (Botstein at al. (1980) Am. J. Hum. Genet. 32:314-331).

The production and use of plant gene-derived probes for use in genetic mapping is described in Bernatzky and Tanksley (1986) Plant Mal. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel at al. in: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield at al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### VI. ASR (Abscisic Acid-, Stress-, and Ripening-Induced) Polypeptide

A preferred method for modulating (preferably, increasing) expression of a nucleic acid encoding an ASR polypeptide is by introducing and expressing in a plant a nucleic acid encoding an ASR polypeptide.

Any reference hereinafter to a “protein useful in the methods of the invention” is taken to mean 3D an ASR polypeptide as defined herein. Any reference hereinafter to a “nucleic acid useful in the methods of the invention” is taken to mean a nucleic acid capable of encoding such an ASR polypeptide. The nucleic acid to be introduced into a plant (and therefore useful in performing the methods of the invention) is any nucleic acid encoding the type of protein which will now be described, hereafter also named “ASR nucleic acid” or “ASR gene”.

A “ASR polypeptide” as defined herein refers the proteins represented by SEQ ID NO: 397 and to homologues (orthologues and paralogues) thereof.

Preferably, the homologues of SEQ ID NO: 397 have a ABA WDS domain (Pfam entry PF02496).

Further Preferably ASR polypeptides of the invention are those having in increasing order of preference at least 50%, 55%, 60%, 65%, 70%, 75%, 80%, 83%, 85%, 87%, 90%, 91%, 92%, 93%, 94%, 95%, 96%, 97%, 98%, 99%, or more sequence identity to any of the polypeptides given in Table Ft The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, ASR polypeptides (at least in their native form), as far as SEQ ID NO: 397 and its homologues are concerned, typically have the capability to increase salt stress resistance of plants. Tools and techniques for expressing the ASR in plants and testing for increased salt stress resistance are well known in the art.

The present invention is illustrated by transforming plants with the nucleic acid sequence represented by SEQ ID NO: 396, encoding the polypeptide sequence of SEQ ID NO: 397. However, performance of the invention is not restricted to these sequences; the methods of the invention may advantageously be performed using any ASR-encoding nucleic acid or ASR polypeptide as defined herein.

Examples of nucleic acids encoding ASR polypeptides may be found in databases known in the art. Such nucleic acids are useful in performing the methods of the invention. Orthologues and paralogues, the terms “orthologues” and “paralogues” being as defined herein, may readily be identified by performing a so-called reciprocal blast search. Typically, this involves a first BLAST involving BLASTing a query sequence (for example using SEQ ID NO: 397) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 396 or SEQ ID NO: 397, the second BLAST would therefore be against Oryza sativa sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants encoding homologues and derivatives of SEQ ID NO: 397 may also be useful in practising the methods of the invention, the terms “homologue” and “derivative” being as defined herein. Also useful in the methods of the invention are nucleic acids encoding homologues and derivatives of orthologues or paralogues of SEQ ID NO: 397. Homologues and derivatives useful in the methods of the present invention have substantially the same biological and functional activity as the unmodified protein from which they are derived.

Further nucleic acid variants useful in practising the methods of the invention include portions of nucleic acids encoding ASR polypeptides, nucleic acids hybridising to nucleic acids encoding ASR polypeptides, splice variants of nucleic acids encoding ASR polypeptides, allelic variants of nucleic acids encoding ASR polypeptides and variants of nucleic acids encoding ASR polypeptides obtained by gene shuffling. The terms hybridising sequence, splice variant, allelic variant and gene shuffling are as described herein.

Nucleic acids encoding ASR polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of SEQ ID NO: 396, or a portion of a nucleic acid encoding an orthologue, paralogue or homologue of SEQ ID NO: 397.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode an ASR polypeptide as defined herein, and have substantially the same biological activity as the amino acid sequences given in SEQ ID NO: 397. Preferably, the portion is a portion of any one of the nucleic acids given in SEQ ID NO: 396, or is a portion of a nucleic acid encoding an orthologue or paralogue of any one of the amino acid sequences given in SEQ ID NO: 396. Preferably the portion is at least 400, 450, 500, 550, 600, 650, 700, 750 consecutive nucleotides in length, the consecutive nucleotides being of SEQ ID NO: 396, or of a nucleic acid encoding an orthologue or paralogue of SEQ ID NO: 397. Most preferably the portion is a portion of the nucleic acid of SEQ ID NO: 396.

Another nucleic acid variant useful in the methods of the invention is a nucleic acid capable of hybridising, under reduced stringency conditions, preferably under stringent conditions, with a nucleic acid encoding an ASR polypeptide as defined herein, or with a portion as defined herein.

According to the present invention, there is provided a method for enhancing yield-related traits in plants, comprising introducing and expressing in a plant a nucleic acid capable of hybridizing to SEQ ID NO: 396, or comprising introducing and expressing in a plant a nucleic acid capable of hybridising to a nucleic acid encoding an orthologue, paralogue or homologue of SEQ ID NO: 396.

Hybridising sequences useful in the methods of the invention encode an ASR polypeptide as defined herein, having substantially the same biological activity as the amino acid sequences given in SEQ ID NO: 397. Preferably, the hybridising sequence is capable of hybridising to SEQ ID NO: 396, or to a portion of any of these sequences, a portion being as defined above, or the hybridising sequence is capable of hybridising to a nucleic acid encoding an orthologue or paralogue of SEQ ID NO: 397.

Another nucleic acid variant useful in the methods of the invention is a splice variant encoding an ASR polypeptide as defined hereinabove, a splice variant being as defined herein.

According to the present invention, there is provided a method for enhancing yield-related traits in plants, comprising introducing and expressing in a plant a splice variant of SEQ ID NO: 396, or a splice variant of a nucleic acid encoding an orthologue, paralogue or homologue of SEQ ID NO: 397.

Another nucleic acid variant useful in performing the methods of the invention is an allelic variant of a nucleic acid encoding an ASR polypeptide as defined hereinabove, an allelic variant being as defined herein.

According to the present invention, there is provided a method for enhancing yield-related traits in plants, comprising introducing and expressing in a plant an allelic variant of SEQ ID NO: 396, or comprising introducing and expressing in a plant an allelic variant of a nucleic acid encoding an orthologue, paralogue or homologue of the amino acid sequences represented by SEQ ID NO: 397.

The allelic variants useful in the methods of the present invention have substantially the same biological activity as the ASR polypeptide of SEQ ID NO: 397. Allelic variants exist in nature, and encompassed within the methods of the present invention is the use of these natural alleles. Gene shuffling or directed evolution may also be used to generate variants of nucleic acids encoding ASR polypeptides as defined above; the term “gene shuffling” being as defined herein.

According to the present invention, there is provided a method for enhancing yield-related traits in plants, comprising introducing and expressing in a plant a variant of SEQ ID NO: 396, or comprising introducing and expressing in a plant a variant of a nucleic acid encoding an orthologue, paralogue or homologue of SEQ ID NO: 397, which variant nucleic acid is obtained by gene shuffling.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding ASR polypeptides may be derived from any natural or artificial source. The nucleic acid may be modified from its native form in composition and/or genomic environment through deliberate human manipulation. Preferably the ASR polypeptide-encoding nucleic acid is from a plant. In the case of SEQ ID NO: 396, the ASR polypeptide encoding nucleic acid is preferably from a monocotyledonous plant, more preferably from the family Poaceae, most preferably the nucleic acid is from Oryza sativa.

The invention also provides hitherto unknown ASR-encoding nucleic acids and ASR polypeptides.

According to a further embodiment of the present invention, there is therefore provided an isolated nucleic acid molecule selected from:
(i) a nucleic acid represented by any one of SEQ ID NO: 401, 403, 405, 407, 409, 411, 413, 415 and 417;
(ii) the complement of a nucleic acid represented by any one of SEQ ID NO: 401, 403, 405, 407, 409, 411, 413, 415 and 417;
(iii) a nucleic acid encoding an ASR polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by any one of SEQ ID NO: 402, 404, 406, 408, 410, 412, 414, 416 and 418.

According to a further embodiment of the present invention, there is also provided an isolated polypeptide selected from:
(i) an amino acid sequence represented by any one of SEQ ID NO: 402, 404, 406, 408, 410, 412, 414, 416 and 418;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented any one of SEQ ID NO: 402, 404, 406, 408, 410, 412, 414, 416 and 418.
(iii) derivatives of any of the amino acid sequences given in (i) or (ii) above.

Performance of the methods of the invention gives plants having enhanced yield-related traits. In particular performance of the methods of the invention gives plants having increased early vigour and increased yield, especially increased biomass and increased seed yield relative to control plants. The terms “yield” and “seed yield” are described in more detail in the “definitions” section herein.

Reference herein to enhanced yield-related traits is taken to mean an increase in early vigour and/or in biomass (weight) of one or more parts of a plant, which may include aboveground (harvestable) parts and/or (harvestable) parts below ground. In particular, such harvestable parts are biomass and/or seeds, and performance of the methods of the invention results in plants having increased early vigour, biomass and/or seed yield relative to the early vigour, biomass or seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially biomass and/or seed yield of plants, relative to control plants, which method comprises modulating expression, preferably increasing expression, in a plant of a nucleic acid encoding an ASR polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding an ASR polypeptide as defined herein. In a particular embodiment, performance of the methods of the present invention gives plants with increased early vigour.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield and/or increased early vigour, relative to control plants grown under comparable conditions. Therefore, according to the present invention, there is provided a method for increasing yield and/or early vigour in plants grown under non-stress conditions or under mild drought conditions, which method comprises increasing expression in a plant of a nucleic acid encoding an ASR polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding an ASR polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding an ASR polypeptide as defined above.

The invention also provides genetic constructs and vectors to facilitate introduction and/or expression in plants of nucleic acids encoding ASR polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding an ASR polypeptide as defined above;
(b) one or more control sequences capable of driving expression of the nucleic acid sequence of (a); and optionally
(c) a transcription termination sequence.

Preferably, the nucleic acid encoding an ASR polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods of the invention. See the “Definitions” section herein for definitions of the various promoter types.

It should be clear that the applicability of the present invention is not restricted to the ASR polypeptide-encoding nucleic acid represented by SEQ ID NO: 396, nor is the applicability of the invention restricted to expression of an ASR polypeptide-encoding nucleic acid when driven by a constitutive specific promoter.

The constitutive promoter is preferably a GOS2 promoter, preferably a GOS2 promoter from rice. Further preferably the constitutive promoter is represented by a nucleic acid sequence substantially similar to SEQ ID NO: 398, most preferably the constitutive promoter is as represented by SEQ ID NO: 398. See the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′ UTR and/or 5′ UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene, Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding an ASR polypeptide as defined hereinabove.

More specifically, the present invention provides a method for the production of transgenic plants having increased enhanced yield-related traits, particularly increased early vigour and/or increased yield, which method comprises:
(i) introducing and expressing in a plant or plant cell an ASR polypeptide-encoding nucleic acid; and
(ii) cultivating the plant cell under conditions promoting plant growth and development.

The nucleic acid of (i) may be any of the nucleic acids capable of encoding an ASR polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hagen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding an ASR polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding an ASR polypeptide is by introducing and expressing in a plant a nucleic acid encoding an ASR polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding ASR polypeptides as described herein and use of these ASR polypeptides in enhancing any of the aforementioned yield-related traits in plants.

Nucleic acids encoding ASR polypeptide described herein, or the ASR polypeptides themselves, may find use in breeding programmes in which a DNA marker is identified which may be genetically linked to an ASR polypeptide-encoding gene. The nucleic acids/genes, or the ASR polypeptides themselves may be used to define a molecular marker. This DNA or protein marker may then be used in breeding programmes to select plants having enhanced yield-related traits as defined hereinabove in the methods of the invention.

Allelic variants of an ASR polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding ASR polypeptides may also be used as probes for genetically and physically mapping the genes that they are a part of, and as markers for traits linked to those genes. Such information may be useful in plant breeding in order to develop lines with desired phenotypes. Such use of ASR polypeptide-encoding nucleic acids requires only a nucleic acid sequence of at least 15 nucleotides in length. The ASR polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the ASR-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the ASR polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### VII. Squamosa Promoter Binding Protein-Like 11 (SPL11) Transcription Factor Polypeptide

Surprisingly, it has now been found that modulating expression in a plant of a nucleic acid encoding a SPL11 polypeptide gives plants having enhanced yield-related traits relative to control plants. According to a first embodiment, the present invention provides a method for enhancing yield-related traits in plants relative to control plants, comprising modulating expression in a plant of a nucleic acid encoding a SPL11 polypeptide.

The present invention also provides hitherto unknown SPL11-encoding nucleic acids and SPL11 polypeptides. These sequences also being useful in performing the methods of the invention.

Therefore according to a further embodiment of the present invention there is provided an isolated nucleic acid molecule comprising:
(i) a nucleic acid represented by SEQ ID NO: 448;
(ii) the complement of a nucleic acid represented by SEQ ID NO: 448;
(iii) a nucleic acid encoding a SPL11 polypeptide having, in increasing order of preference, at least 70%, 75%, 80%, 85%, 90%, 95%, 96%, 97%, 98%, 99% or more sequence identity to the amino acid sequence represented by SEQ ID NO: 449, and having in increasing order of preference at least 70%, 75%, 80%, 85%, 90%, 95%, 96%, 97%, 98%, 99% or more sequence identity to SEQ ID NO: 465: SYCOVEGCRTDLSSAKDYHRKHRVCEPHSKAPKVVVAGLERRFC QQCSRFHGLAEFDQKKKSCRRRLNDHNARRRKPQPEAL (which represents the SBP domain in SEQ ID NO: 449);
(iv) a nucleic acid hybridising under stringent conditions to SEQ ID NO: 448.

Furthermore, there is also provided an isolated polypeptide comprising:
(i) an amino acid sequence represented by SEQ ID NO: 449;
(ii) an amino acid sequence having, in increasing order of preference, at least 70%, 75%, 80%, 85%, 90%, 95%, 96%, 97%, 98%, 99% or more sequence identity to the amino acid sequence represented by SEQ ID NO: 449, and having in increasing order of preference at least 70%, 75%, 80%, 85%, 90%, 95%, 96%, 97%, 98%, 99% or more sequence identity to SEQ ID NO: 465: SYCQVEGCRTDLSSAKDYHRKHRVCEPHSKAPKVVVAGLERRFCQQCSRFHG LAEFDQKKKSCRRRLNDHNARRRKPQPEAL (which represents the SBP domain in SEQ ID NO: 449).
(iii) derivatives of any of the amino acid sequences given in (i) or (ii) above.

A preferred method for modulating (preferably, increasing) expression of a nucleic acid encoding a SPL11 polypeptide is by introducing and expressing in a plant a nucleic acid encoding a SPL11 polypeptide.

Any reference hereinafter to a “protein useful in the methods of the invention” is taken to mean a SPL11 polypeptide as defined herein. Any reference hereinafter to a “nucleic acid useful in the methods of the invention” is taken to mean a nucleic acid capable of encoding such a SPL11 polypeptide. The nucleic acid to be introduced into a plant (and therefore useful in performing the methods of the invention) is any nucleic acid encoding the type of protein which will now be described, herein also named “SPL11 nucleic acid” or “SPL11 gene”.

A SPL11 polypeptide as defined herein refers to a polypeptide comprising a Squamosa Binding Protein (SBP) domain, such domain having in increasing order of preference at least 70%, 75%, 80%, 85%, 90%, 95%, 97% or more sequence identity to any one of the SBP domains as represented by SEQ ID NO: 456 to SEQ ID NO: 468 or SEQ ID NO: 478.

An “SPL11 polypeptide” as defined herein comprises the protein represented by SEQ ID NO: 448 which identical to SEQ ID NO: 172 and to homologues (orthologues and paralogues) thereof. Preferably, the homologues of SEQ ID NO: 172 have a DNA binding domain. SPL11 polypeptides can be found in specialized databases such as Pfam, (Finn et al. Nucleic Acids Research (2006) Database Issue 34:D247-D251). Pfam compiles a large collection of multiple sequence alignments and hidden Markov models (HMM) covering many common protein domains and families and is available through the Sanger Institute in the United Kingdom.

The gathering cutoff threshold of the SBP domain in the Pfam HMM_fs and Pfam HMM_ls models is 25.0. Trusted matches as considered in the Pfam database are those sequences scoring higher than the gathering cut-off threshold. However potential matches, comprising true SBP domains, may still fall under the gathering cut-off. Preferably a SPL11 polypeptide is a protein having one or more domains in their sequence that exceed the gathering cutoff of the Pfam protein domain family PF03110, known as SBP domain.

Alternatively, a SBP domain in a polypeptide may be identified by performing a sequence comparison with known polypeptides comprising a SBP domain and establishing the similarity in the region of the SBP domain. The sequences may be aligned using any of the methods well known in the art such as Blast algorithms. The probability for the alignment to occur with a given sequence is taken as basis for identifying similar polypeptides. A parameter that is typically used to represent such probability is called e-value. The E-value is a measure of the reliability of the S score. The S score is a measure of the similarity of the query to the sequence shown. The e-value describes how often a given S score is expected to occur at random. The e-value cut-off may be as high as 1.0. The typical threshold for a good e-value from a BLAST search output using an SPL11 polypeptide as query sequence can is lower than e⁵(=10⁻⁵), 1.e⁻¹⁵, 1.e⁻¹⁵, 1.e⁻²⁰, 1.e⁻²⁵, 1.e⁻⁵⁵, 1.e⁻⁷⁵, 1.e⁻¹⁰⁰, 1.e⁻²⁰⁰, 1.e⁻³⁰⁰, 1.e⁻⁴⁰⁰, 1.e⁻⁵⁰⁰, 1.e⁻⁶⁰⁰, 1.e⁻⁷⁰⁰ and 1.e⁻⁸⁰⁰. Preferably SPL11 polypeptides of the invention comprise a sequence having in increasing order of preference an e-value lower than e⁵(=10⁻⁵), 1.e⁻²⁰, 1.e⁻²⁵, 1.e⁻⁵⁰, 1.e⁻⁷⁵, 1.e⁻¹⁰⁰, 1.e⁻²⁰⁰, 1.e³⁰⁰, 1.e⁻⁴⁰⁰, 1.e⁻⁵⁰⁰, 1.e⁻⁶⁰⁰, 1.e⁻⁷⁰⁰ and 1.e⁻⁸⁰⁰ in an alignment with a SBP domain found in a known SPL11 polypeptide.

Examples of SPL11 polypeptides useful in the methods of the invention are given in Table G1. The amino acid coordinates of the SBP domain in the representative SPL11 protein of Table G1 are given in Table G4 and the domain sequence is represented by SEQ ID NO: 456 to SEQ ID NO: 468. A consensus sequence of the SBP domains present in SPL11 polypeptides is given in SEQ ID NO: 478.

Preferred SPL11 polypeptides of the invention are those having in increasing order of preference at least 70%, 75%, 80%, 85%, 90%, 95%, or more sequence identity to any of the polypeptides given in Table G2.

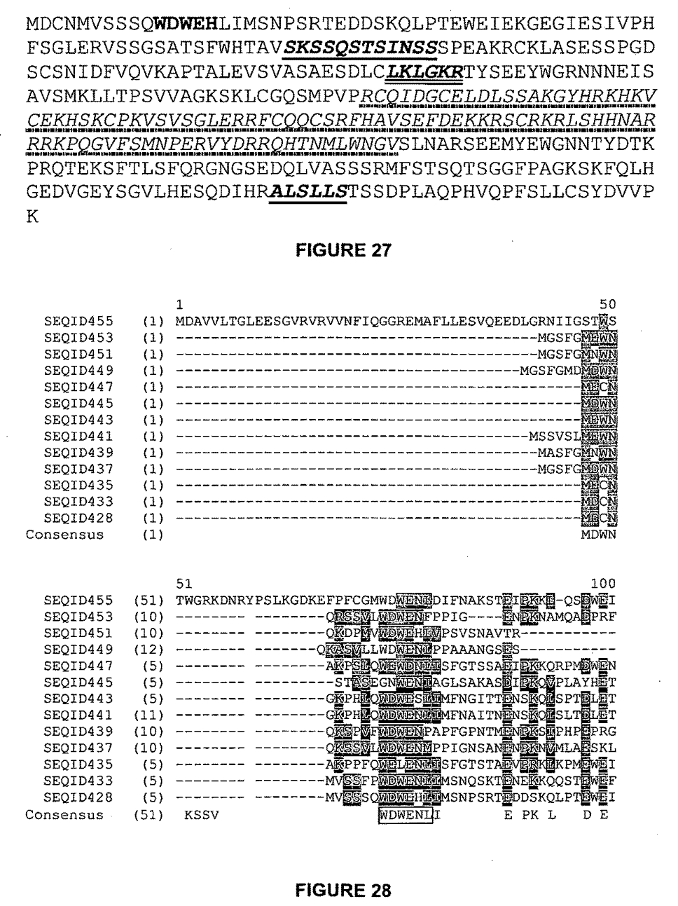

Typically SPL11 polypeptides may comprise in addition to the SBP domain one or more of the following conserved motifs at conserved positions in the sequence relative to the SBP domain: (i) Motif 1 as represented by SEQ ID NO: 469, (ii) Motif 2 is a serine rich region typically found at the N-terminal end of the SBP domain and can be represented by SEQ ID NO: 470; (iii) Motif 3 as presented by SEQ ID: 471 which is typically encoded by nucleotides comprised within the SPL11 polynucleotide region targeted by members of the miR156 microRNA family; (iv) Motif 4 as represented by SEQ ID NO: 472. FIG. 27 shows the conserved motifs and their relative position in the SPL11 polypeptide sequence represented by SEQ ID NO: 428.

Therefore, preferred SPL11 polypeptides useful in the method of the invention, comprise in addition to the SBP domain any one or more of the following conserved motifs:
(i) Motif 1 as represented by SEQ ID NO: 469 wherein any conservative amino acid substitution and/or 1 or 2 non conservative substitutions are allowed,
(ii) Motif 2 as represented by SEQ ID NO: 470 wherein any amino acid substitution is allowed, provided that at least 4 amino acids have a polar side chain, preferably serine or threonine, and provided that this motif is located at the N-terminal end of the SBP domain;
(iii) Motif 3 as represented by SEQ ID: 471, wherein 1 or 2 mismatches are allowed;
(iv) Motif 4 as represented by SEQ ID: 472, wherein 1, 2 or 3 mismatches are allowed.

Examples of conservative amino acid substitutions are given in the background section. Typically amino acids comprised in polypeptides are alpha amino acids having an amine and a carboxyl group attached to the same carbon, the alpha carbon, which amino acid molecules often comprise a side chain attached to the alpha carbon. Table 3 shows the classification of amino acids based on the physical and biochemical properties of the side chain.

TABLE 3

Classification of amino acids according to the
side chain properties.

| | | | | | |
|---|---|---|---|---|---|
| | | | Side chain | Side chain acidity | Hydropathy |
| Amino Acid | 3-Letter | 1-Letter | polarity | or basicity | index |

| | | | | | |
|---|---|---|---|---|---|
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | polar | neutral | 2.5 |
| Glutamic acid | Glu | E | polar | acidic | −3.5 |
| Glutamine | Gln | Q | polar | neutral | −3.5 |
| Histidine | His | H | polar | basic | −3.2 |
| Lysine | Lys | K | polar | basic | −3.9 |
| Serine | Ser | S | polar | neutral | −0.8 |
| Threonine | Thr | T | polar | neutral | −0.7 |
| Tyrosine | Tyr | Y | polar | neutral | −1.3 |
| Alanine | Ala | A | nonpolar | neutral | 1.8 |
| Glycine | Gly | G | nonpolar | neutral | −0.4 |
| Isoleucine | Ile | I | nonpolar | neutral | 4.5 |
| Leucine | Leu | L | nonpolar | neutral | 3.8 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Tryptophan | Trp | W | nonpolar | neutral | −0.9 |
| Valine | Val | V | nonpolar | neutral | 4.2 |

Examples of SPL11 polypeptides comprising one or more of the conserved motifs Motif 1 to Motif 4 are given in Example 62. FIG. 28 shows the position of the conserved motifs in those SPL11 polypeptides.

Preferably, the SPL11 polypeptide of the invention when used in the construction of a phylogenetic tree, such as the one depicted in FIG. 29, clusters within the 33 group comprising the amino acid sequence represented by SEQ ID NO: 428 (named AtSPL11 in FIG. 29) rather than with any other group.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244, interPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hub et al., Nucl. Acids. Res. 32:D134-D137, (2004), or Pram (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, SPL11 polypeptides (at least in their native form) may typically have DNA binding activity, in particular they may bind DNA fragments comprising the SBP domain DNA binding box as represented by SEQ ID NO: 49. Typically the SBP domain DNA binding box is found in gene promoters of plant origin such as the SQUAMOSA gene. Fragments comprising the SBP domain DNA binding box are preferably more than 10, 15, 20, 25, 100, 200, 500, 1000, 2000 base pairs long. Methods to determine DNA binding of SBP domain containing proteins are applicable to SPL11 polypeptides and are known in the art (Klein at al. Mol Gen Genet. 1996, 15; 250(1):7-16; Yamasaki et al. 2004 J Mol. Biol. 2004, 12; 337(1):49-63).

Preferred SPL11 polypeptides of the invention are those having DNA binding activity, more preferably those binding a DNA fragment comprising SEQ ID NO: 475.

The present invention is illustrated by transforming plants with the nucleic acid sequence represented by SEQ ID NO: 427, encoding the polypeptide sequence of SEQ ID NO: 428. However, performance of the invention is not restricted to these sequences; the methods of the invention may advantageously be performed using any SPL11 encoding nucleic acid or SPL11 polypeptide as defined herein.

Examples of nucleic acids encoding SPL11 polypeptides are given in Table G1 of Example 62 herein. Such nucleic acids are useful in performing the methods of the invention. The amino acid sequences given in Table G1 of Example 62 are example sequences of orthologues and paralogues of the SPL11 polypeptide represented by SEQ ID NO: 428, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table G1 of Example 62) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 427 or SEQ ID NO: 428, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table G1 of Example 62, the terms “homologue” and “derivative” being as defined herein. Also useful in the methods of the invention are nucleic acids encoding homologues and derivatives of orthologues or paralogues of any one of the amino acid sequences given in Table G1 of Example 62. Homologues and derivatives useful in the methods of the present invention have substantially the same biological and functional activity as the unmodified protein from which they are derived.

Further nucleic acid variants useful in practising the methods of the invention include portions of nucleic acids encoding SPL11 polypeptides, nucleic acids hybridising to nucleic acids encoding SPL11 polypeptides, splice variants of nucleic acids encoding SPL11 polypeptides, allelic variants of nucleic acids encoding SPL11 polypeptides and variants of nucleic acids encoding SPL11 polypeptides obtained by gene shuffling. The terms hybridising sequence, splice variant, allelic variant and gene shuffling are as described herein.

A preferred nucleic acid variant useful in practising the methods of the invention is a nucleic acid encoding a SPL11 polypeptide, which is microRNA insensitive, further preferably the nucleic acid is insensitive to microRNAs belonging to the miR156 family. MicroRNAs target nucleic acids (RNA in particular) for destruction typically causing a reduction or inhibition of the accumulation of the targeted RNA. Targeting requires hybridisation between the microRNA and the targeted nucleic acid (RNA) in a very specific region, called the miR (microRNA) target site, which comprises a sequence complementary to a portion of the mature microRNA gene. Typically microRNA insensitive nucleic acids comprise a sequence having in increasing order of preference 1, 2, 3, 4, 5 or more mismatches in an alignment to the relevant microRNA molecule in the miR target site. MicroRNA insensitive nucleic acids may accumulated in a cell to levels in increasing order of preference 5, 10, 20, 30, 40, 50 times or higher than the corresponding nucleic acid targeted by the relevant MicroRNA, which typically comprises 100% sequence complementary in the miR target site.

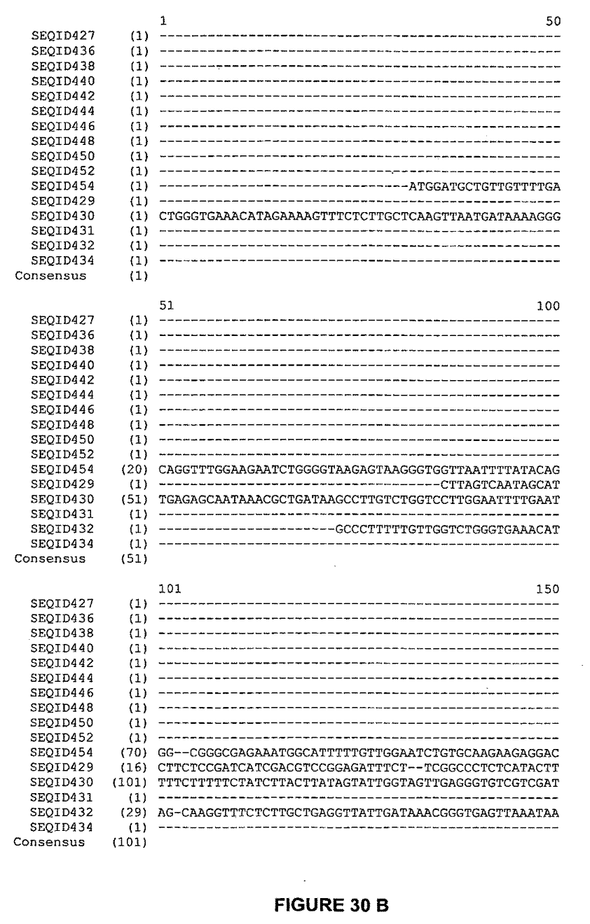

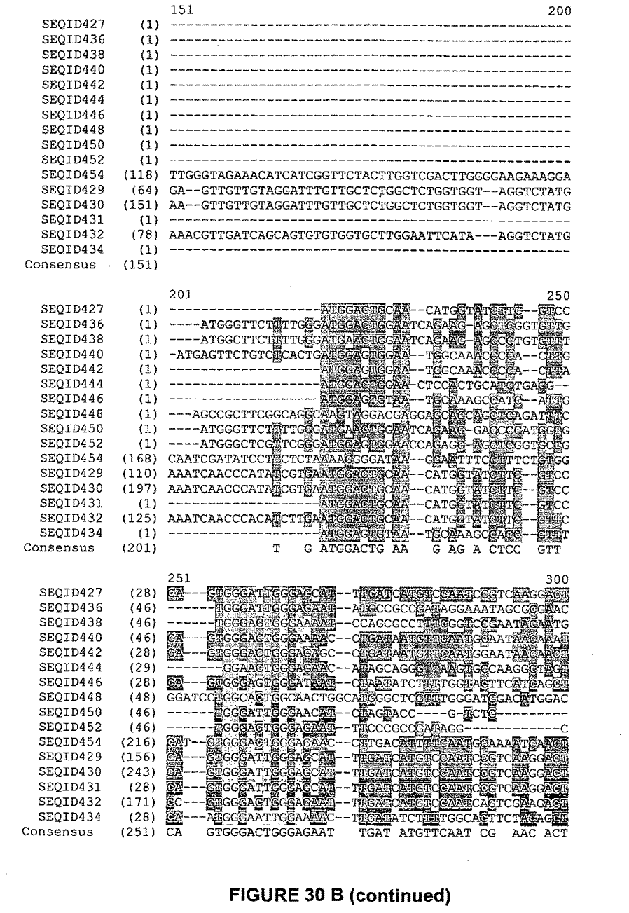

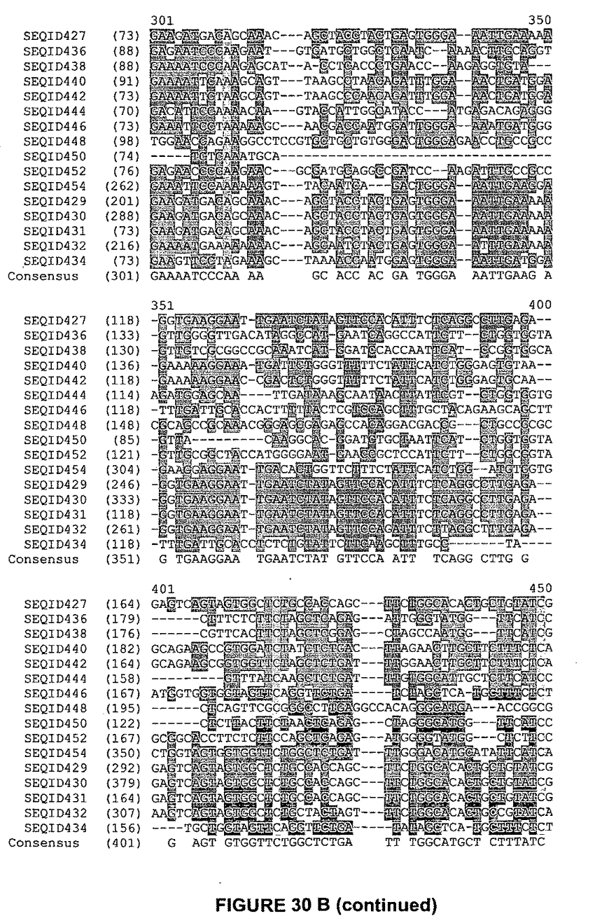

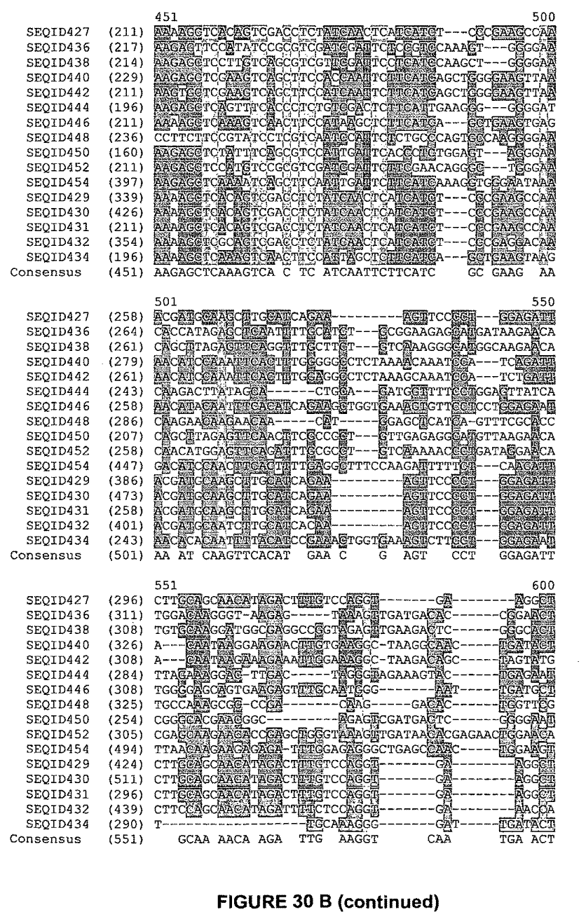

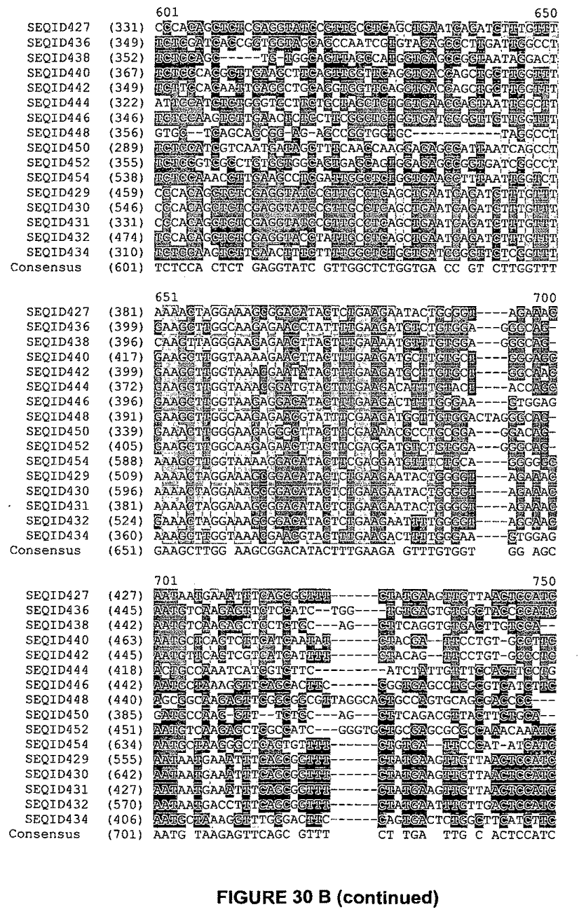

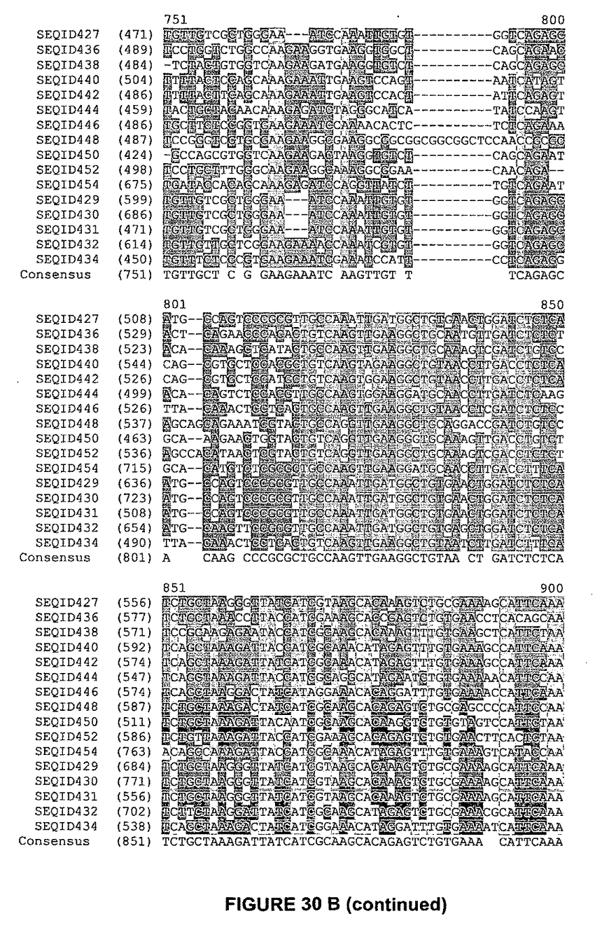

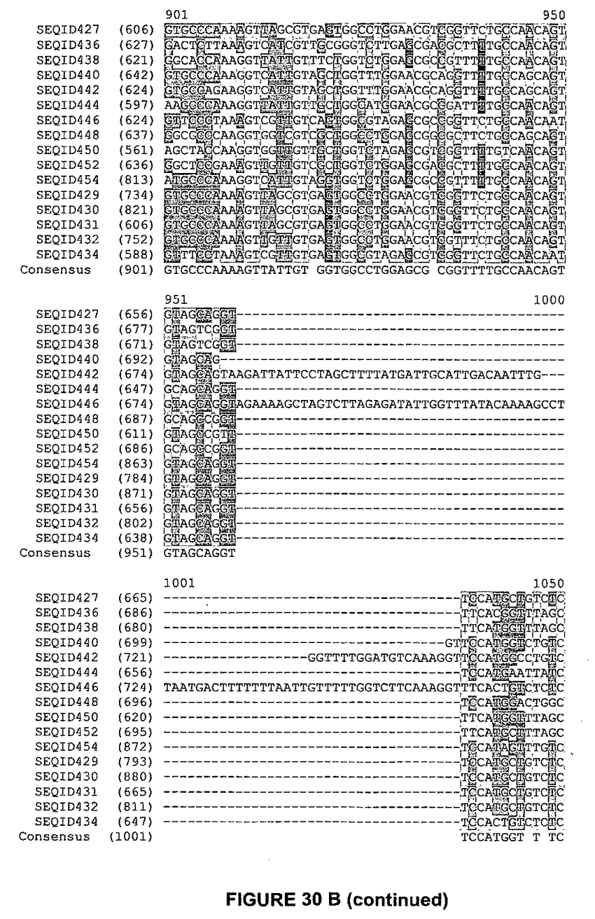

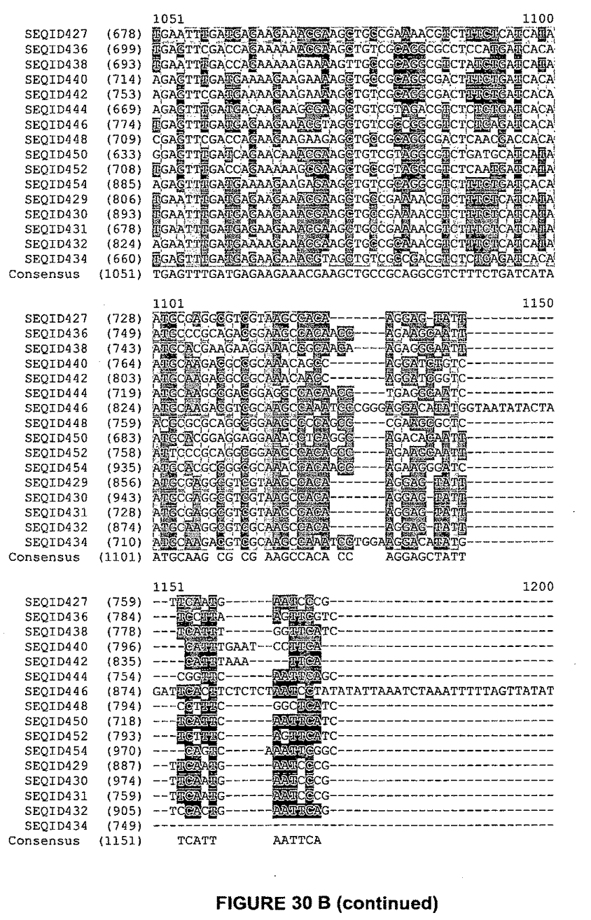

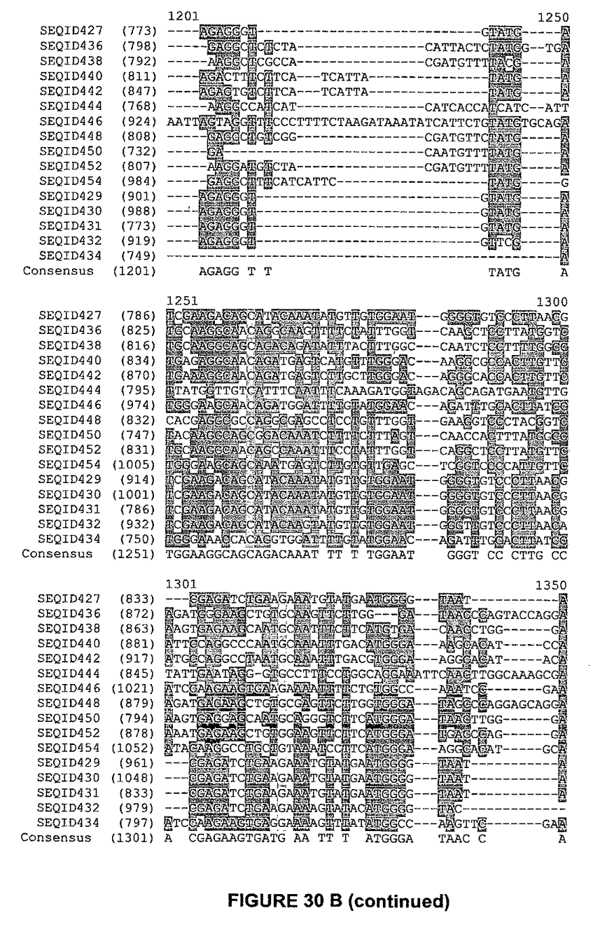

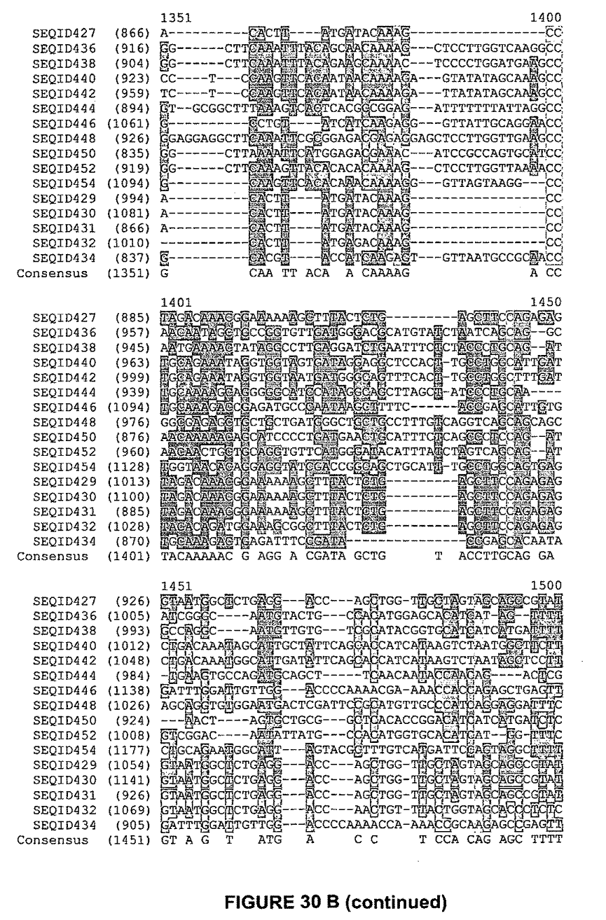

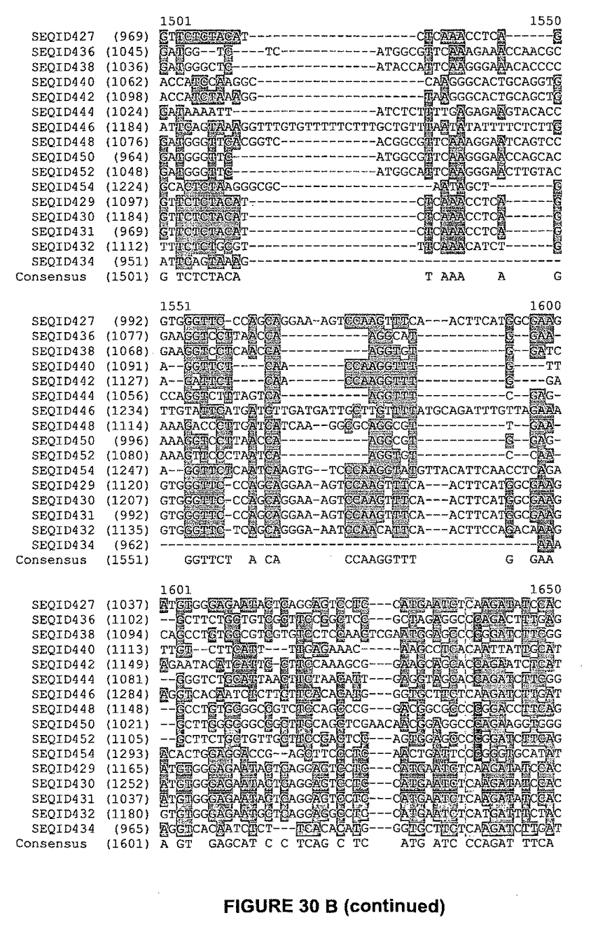

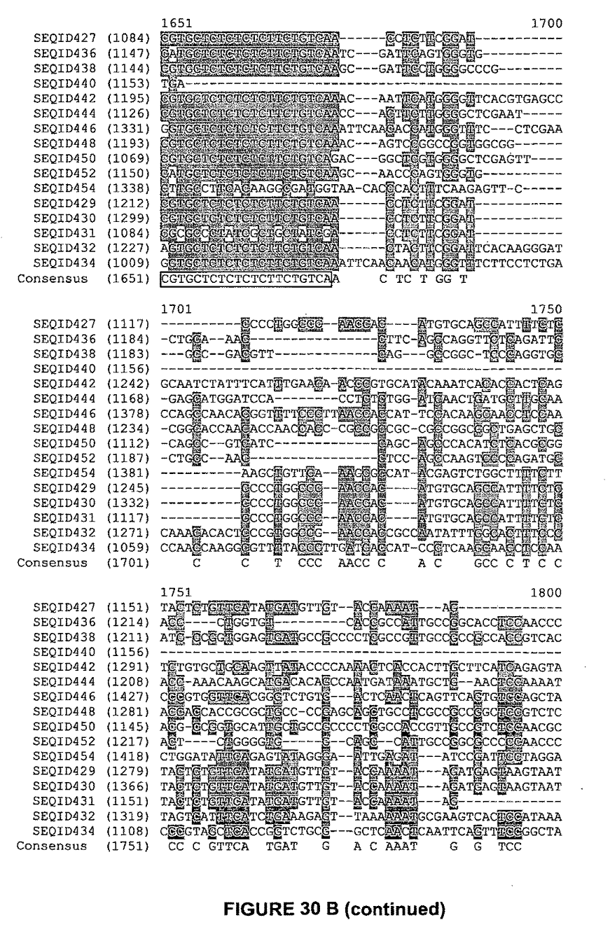

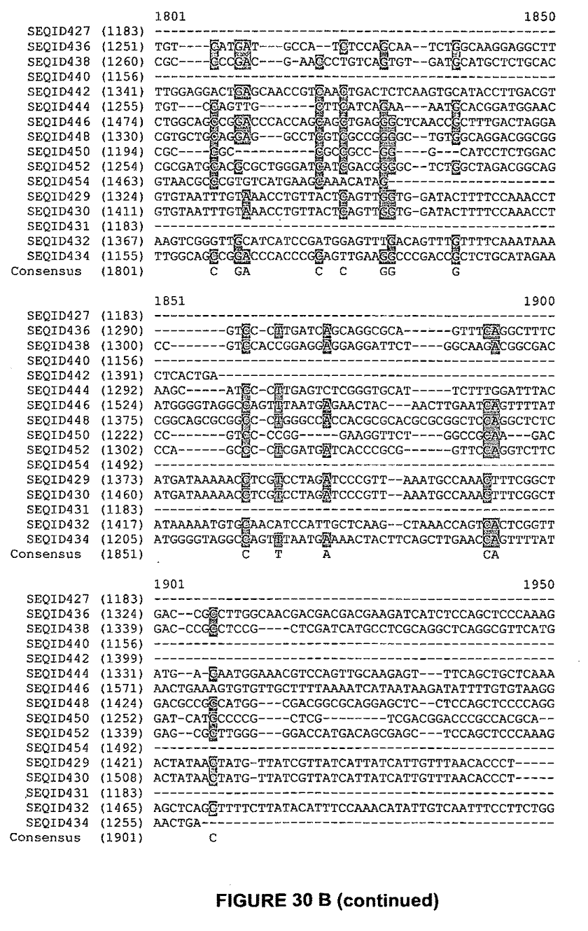

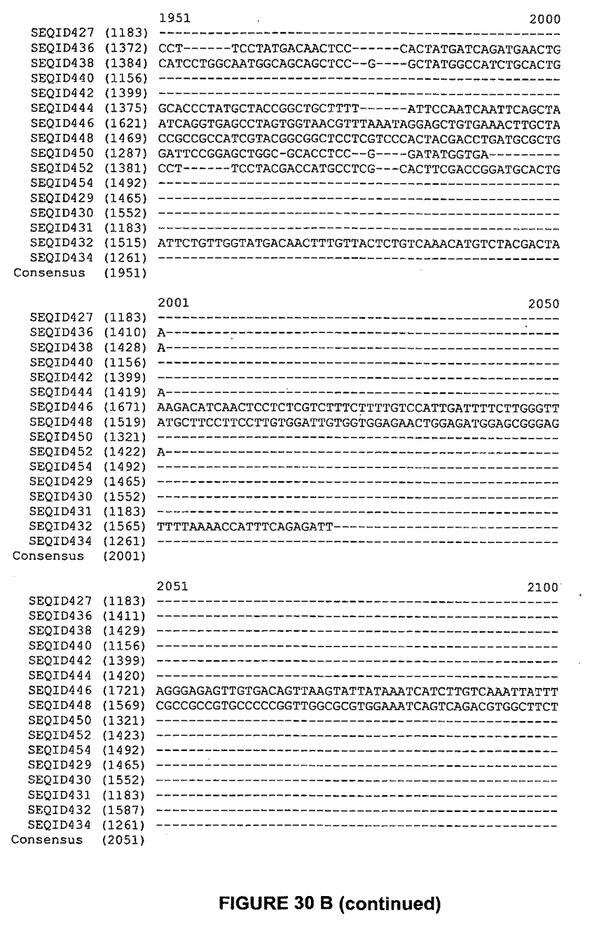

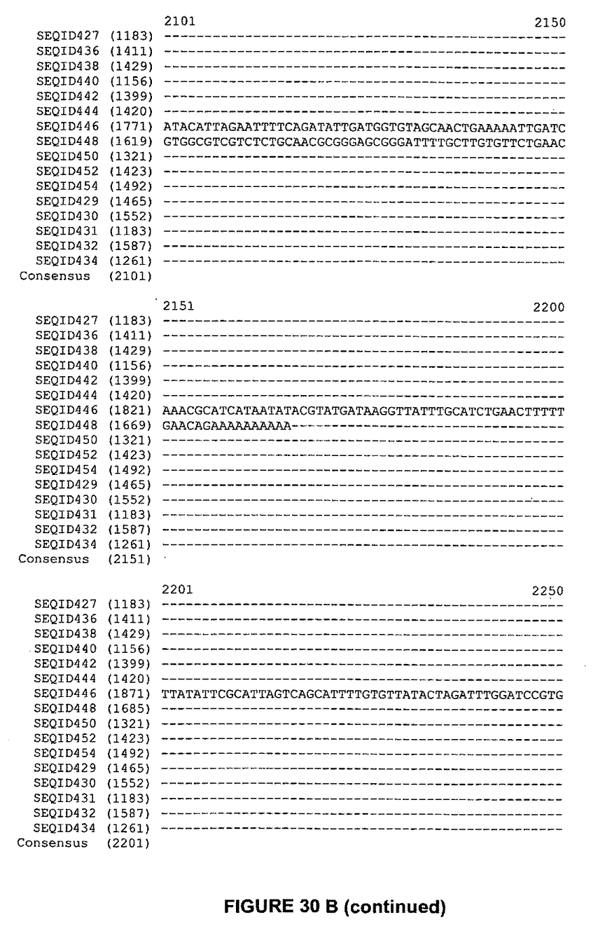

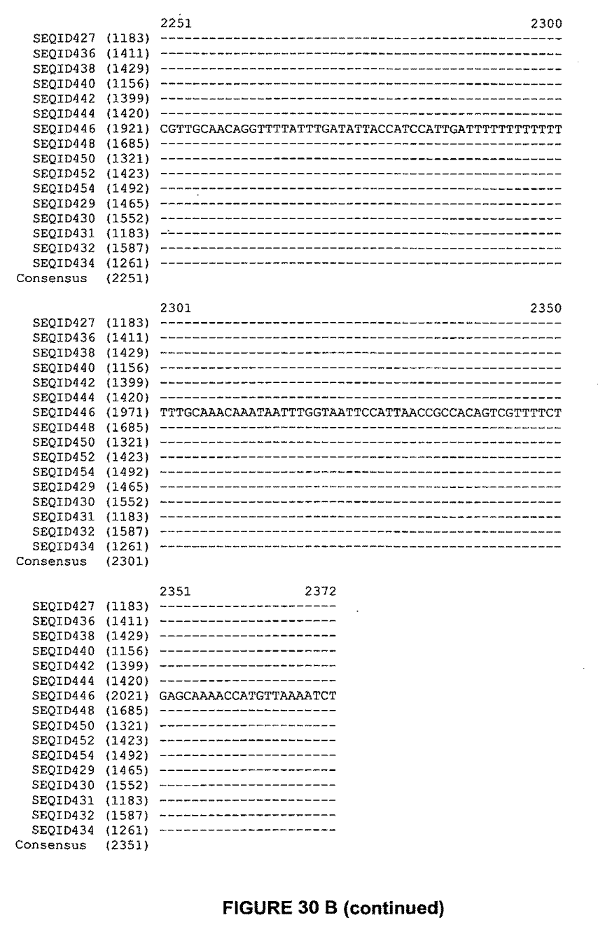

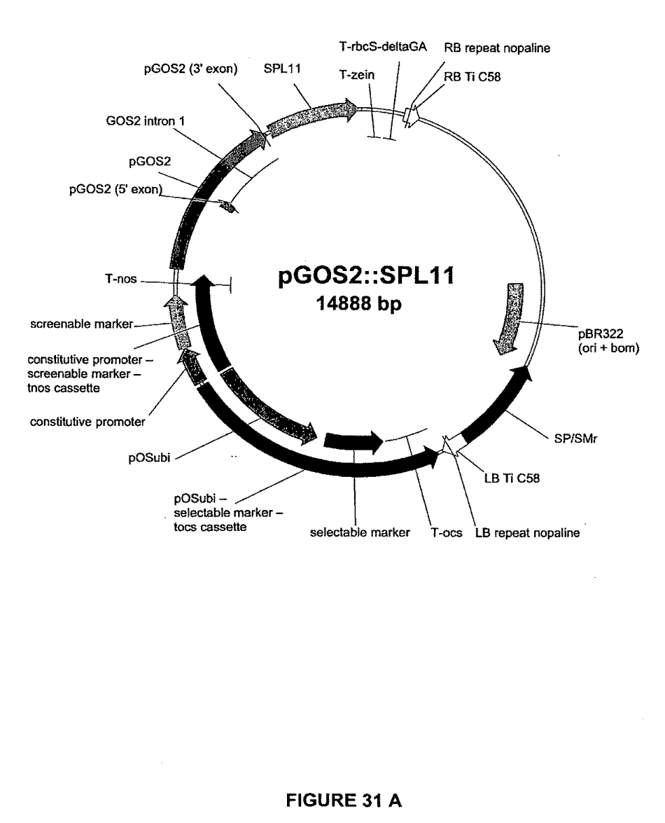

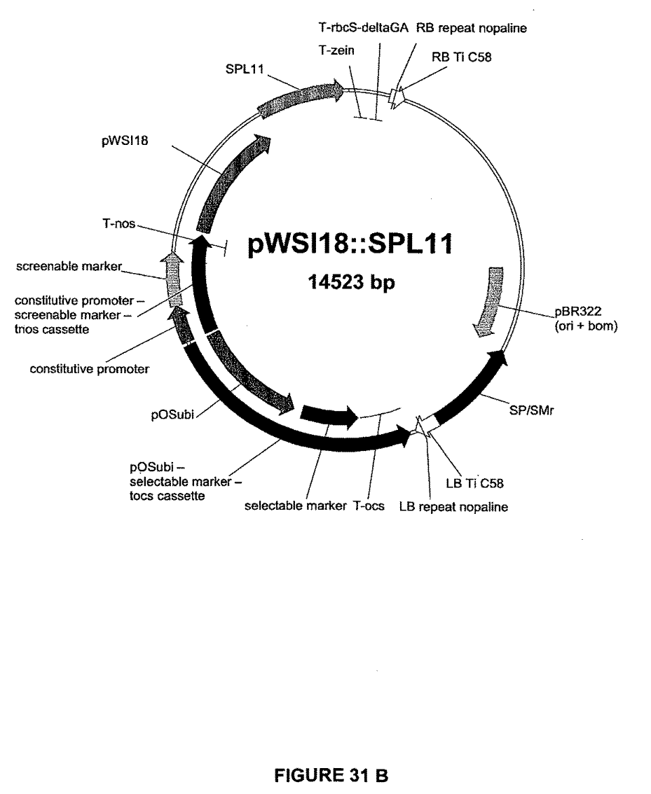

The miR156 family has been described earlier and a compilation of the microRNAs including the miR156 family members can be found at miRBase database (Griffiths-Jones et al. 2006 Nucleic Acids Research, 2006, Vol. 34, Database issue 0140-D144). The miRBase database is maintained by the The Wellcome Trust Sanger Institute, in Cambridge, UK. The miR156 target site in a SPL11 nucleic acid is complementary to the mature sequence of miR156 microRNAs. An example of mature sequences of the miR156 family members from rice and their corresponding target sites on rice SPL nucleic acids is given in FIG. 30 A. FIG. 31B shows an alignment of representative SPL11 nucleic acids (in DNA form), in which the target site of miR156 in the corresponding ribonucleic acids is indicated. An example of a miR156 insensitive SPL11 nucleic acid encoding SEQ ID NO: 428 is represented by SEQ ID NO: 431. Further examples of miR156 insensitive SPL11 nucleic acids are represented by SEQ ID 440 and SEQ ID NO: 454, the latter lacking the miR156 target site.

Preferably a SPL11 nucleic acid useful in the methods of the invention has 1, 2, 3, 4 or more mismatches in the miR156 target site or lack the target site of the miR156 gene. Examples of such SPL11 nucleic acids are given in FIG. 31B. Further preferably is a nucleic acid as represented by SEQ ID NO: 431, SEQ ID 440 and SEQ ID NO: 454.

Nucleic acids encoding SPL11 polypeptides need not be full-length nucleic adds, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table G1 of Example 62, or a portion of a nucleic acid encoding an orthologue, paralogue or homologue of any of the amino acid sequences given in Table G1 of Example 62.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a SPL11 polypeptide as defined herein, and have substantially the same biological activity as the amino acid sequences given in Table G1 of Example 62. Preferably, the portion is a portion of any one of the nucleic acids given in Table G1 of Example 62, or is a portion of a nucleic acid encoding an orthologue or paralogue of any one of the amino acid sequences given in Table G1 of Example 62. Preferably the portion is at least 70, 100, 200, 500, 550, 600, 650, 700, 750, 800, 850, 900, 950, 1000, 1500, 2000, 2500, 3000, 3500, 4000, 4500, 5000 consecutive nucleotides in length, the consecutive nucleotides being of any one of the nucleic acid sequences given in Table G1 of Example 62, or of a nucleic acid encoding an orthologue or paralogue of any one of the amino acid sequences given in Table G1 of Example 62. Preferably the portion encodes at least an SBP domain. Most preferably the portion is a portion of the nucleic acid of SEQ ID NO: 427. Preferably, the portion encodes an amino acid sequence which when used in the construction of a phylogenetic tree, such as the one depicted in FIG. 29, clusters with the group of SPL11 polypeptides comprising the amino acid sequence represented by SEQ ID NO: 428 (AtSPL11) rather than with any other group.

Another nucleic acid variant useful in the methods of the invention is a nucleic acid capable of hybridising, under reduced stringency conditions, preferably under stringent conditions, with a nucleic acid encoding a SPL11 polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table G1 of Example 62, or comprising introducing and expressing in a plant a nucleic acid capable of hybridising to a nucleic acid encoding an orthologue, paralogue or homologue of any of the nucleic acid sequences given in Table G1 of Example 62.

Hybridising sequences useful in the methods of the invention encode a SPL11 polypeptide as defined herein, and have substantially the same biological activity as the amino acid sequences given in Table G1 of Example 62. Preferably, the hybridising sequence is capable of hybridising to any one of the nucleic acids given in Table G1 of Example 62, or to a portion of any of these sequences, a portion being as defined above, or wherein the hybridising sequence is capable of hybridising to a nucleic acid encoding an orthologue or paralogue of any one of the amino acid sequences given in Table G1 of Example 62. Most preferably, the hybridising sequence is capable of hybridising to a nucleic acid as represented by SEQ ID NO: 427 or to a portion thereof.

Preferably, the hybridising sequence encodes an amino acid sequence which when used in the construction of a phylogenetic tree, such as the one depicted in FIG. 29, clusters with the group of SPL11 polypeptides comprising the amino acid sequence represented by SEQ ID NO: 428 (AtSPL11) rather than with any other group.

Another nucleic acid variant useful in the methods of the invention is a splice variant encoding a SPL11 polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table G1 of Example 62, or a splice variant of a nucleic acid encoding an orthologue, paralogue or homologue of any of the amino acid sequences given in Table G1 of Example 62. Examples of spliced variants of the gene encoding SEQ ID NO: 428 are represented by SEQ ID NO: 427; SEQ ID NO: 429 and SEQ ID NO: 430.

Preferred splice variants are splice variants of a nucleic acid represented by SEQ ID NO: 427, or a splice variant of a nucleic acid encoding an orthologue or paralogue of SEQ ID NO: 428. Preferably, the amino acid sequence encoded by the splice variant, when used in the construction of a phylogenetic tree, such as the one depicted in FIG. 29, clusters with the group of SPL11 polypeptides comprising the amino acid sequence represented by SEQ ID NO: 428 (AtSPL11) rather than with any other group.

Another nucleic acid variant useful in performing the methods of the invention is an allelic variant of a nucleic acid encoding a SPL11 polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table G1 of Example 62, or comprising introducing and expressing in a plant an allelic variant of a nucleic acid encoding an orthologue, paralogue or homologue of any of the amino acid sequences given in Table G1 of Example 62.

The allelic variants useful in the methods of the present invention have substantially the same biological activity as the SPL11 polypeptide of SEQ ID NO: 428 and any of the amino acids depicted in Table G1 of Example 62. Allelic variants exist in nature, and encompassed within the methods of the present invention is the use of these natural alleles. Preferably, the allelic variant is an allelic variant of SEQ ID. NO: 427 or an allelic variant of a nucleic acid encoding an orthologue or paralogue of SEQ ID NO: 428. Preferably, the amino acid sequence encoded by the allelic variant, when used in the construction of a phylogenetic tree, such as the one depicted in FIG. 29, clusters with the SPL11 polypeptides comprising the amino acid sequence represented by SEQ ID NO: 428 (AtSPL11) rather than with any other group.

Gene shuffling or directed evolution may also be used to generate variants of nucleic acids encoding SPL11 polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table G1 of Example 62, or comprising introducing and expressing in a plant a variant of a nucleic acid encoding an orthologue, paralogue or homologue of any of the amino acid sequences given in Table G1 of Example 62, which variant nucleic acid is obtained by gene shuffling.

Preferably, the amino acid sequence encoded by the variant nucleic acid obtained by gene shuffling, when used in the construction of a phylogenetic tree such as the one depicted in FIG. 29, clusters with the group of SPL11 polypeptides comprising the amino acid sequence represented by SEQ ID NO: 428 (AtSPL11) rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding SPL11 polypeptides may be derived from any natural or artificial source. The nucleic acid may be modified from its native form in composition and/or genomic environment through deliberate human manipulation. Preferably the SPL11 polypeptide-encoding nucleic acid is from a plant, further preferably from a dicotyledonous plant, more preferably from the family Brassicaceae, most preferably the nucleic acid is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased yield relative to the yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a SPL11 polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a SPL11 polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang at al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a SPL11 polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a SPLIT polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a SPL11 polypeptide as defined above.

The invention also provides genetic constructs and vectors to facilitate introduction and/or expression in plants of nucleic acids encoding SPL11 polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a SPL11 polypeptide as defined above;
(b) one or more control sequences capable of driving expression of the nucleic acid sequence of (a); and optionally
(c) a transcription termination sequence.

Preferably, the nucleic acid encoding a SPL11 polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the SPL11 polypeptide-encoding nucleic acid represented by SEQ ID NO: 427, nor is the applicability of the invention restricted to expression of a SPL11 polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 476, most preferably the constitutive promoter is as represented by SEQ ID NO: 476. See the “Definitions” section herein for further examples of constitutive promoters.

In an alternative embodiment, a seed specific promoter is used. The seed specific promoter is preferably ABA (abcisic acid) inducible, it is preferably the WSI18 promoter, preferably the WSI18 from rice. Further preferably the constitutive promoter is represented by a nucleic acid sequence substantially similar to SEQ ID NO: 477. See the “Definitions” section herein for further examples of seed specific promoters.

It should be clear that the promoters useful in the methods of the invention are not limited to those specified in the abovementioned embodiments.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits relative to control plants, comprising introduction and expression in a plant of any nucleic acid encoding a SPL11 polypeptide as defined hereinabove.

More specifically, the present invention provides a method for the production of transgenic plants having increased enhanced yield-related traits, particularly increased (seed) yield, which method comprises:
(i) introducing and expressing in a plant or plant cell a SPL11 polypeptide-encoding nucleic acid; and
(ii) cultivating the plant cell under conditions promoting plant growth and development.

The nucleic acid of (i) may be any of the nucleic acids capable of encoding a SPL11 polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/6r phenotypic characteristic(s) as those produced by the parent in the methods according to the invention.

The invention also includes host cells containing an isolated nucleic acid encoding a SPL11 polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a SPL11 polypeptide is by introducing and expressing in a plant a nucleic acid encoding a SPL11 polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding SPL11 polypeptides as described herein and use of these SPL11 polypeptides in enhancing any of the aforementioned yield-related traits in plants.

Nucleic acids encoding SPL11 polypeptides described herein, or the SPL11 polypeptides themselves, may find use in breeding programmes in which a DNA marker is identified, which may be genetically linked to a SPL11 polypeptide-encoding gene. The nucleic acids/genes, or the SPL11 polypeptides themselves may be used to define a molecular marker. This DNA or protein marker may then be used in breeding programmes to select plants having enhanced yield-related traits as defined hereinabove in the methods of the invention.

Allelic variants of a SPL11 polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding SPL11 polypeptides may also be used as probes for genetically and physically mapping the genes that they are a part of, and as markers for traits linked to those genes. Such information may be useful in plant breeding in order to develop lines with desired phenotypes. Such use of SPL11 polypeptide-encoding nucleic acids requires only a nucleic acid sequence of at least 15 nucleotides in length. The SPL11 polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the SPL11-encoding nucleic acids. The resulting banding patterns may then be subjected to genetic analyses using computer programs such as MapMaker (Lander et. al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the SPL11 polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant MeI. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

The present invention will now be described in reference to the following items:
1. A method for enhancing yield-related traits in plants relative to control plants, comprising modulating expression in a plant of a nucleic acid encoding an LBD polypeptide, wherein said LBD polypeptide comprises a DUF206 domain.
2. Method according to item 1, wherein said LBD polypeptide comprises one or more of the following motifs:

| | |
|---|---|
| (i) Motif 1: MSCNGCRXLRKGCX, | (SEQ ID NO: 5) |
| (ii) Motif 2: QXXATXFXAKFXGR, | (SEQ ID NO: 6) |
| (iii) Motif 3: FXSLLXEAXG | (SEQ ID NO: 7) |

3. Method according to item 1 or 2, wherein said modulated expression is effected by introducing and expressing in a plant a nucleic acid encoding a LBD polypeptide.
4. Method according to any preceding item, wherein said nucleic acid encoding a LBD polypeptide encodes any one of the proteins listed in Table A1 or is a portion of such a nucleic acid, or a nucleic acid capable of hybridising with such a nucleic acid.
5. Method according to any preceding item, wherein said nucleic acid sequence encodes an orthologue or paralogue of any of the proteins given in Table A1.
6. Method according to any preceding item, wherein said enhanced yield-related traits comprise increased yield, preferably increased biomass and/or increased seed yield relative to control plants.
7. Method according to any one of items 1 to 6, wherein said enhanced yield-related traits are obtained under non-stress conditions.
8. Method according to any one of items 1 to 6, wherein said enhanced yield-related traits are obtained under conditions of nitrogen deficiency.
9. Method according to any one of items 3 to 8, wherein said nucleic acid is operably linked to a constitutive promoter, preferably to a GOS2 promoter, most preferably to a GOS2 promoter from rice.
10. Method according to any preceding item, wherein said nucleic acid encoding a LBD polypeptide is of plant origin, preferably from a dicotyledonous plant, further preferably from the family Brassicaceae, more preferably from the genus Arabidopsis, most preferably from Arabidopsis thaliana.
11. An isolated nucleic acid molecule comprising any one of the following features:
   (i) a nucleic acid represented by SEQ ID NO: 69;
   (ii) a nucleic acid or fragment thereof that is complementary to any one of the SEQ ID NOs given in (i);
   (iii) a nucleic acid encoding a LBD polypeptide having, in increasing order of preference, at least 60%, 65%, 70%, 75%, 80%, 85%, 90%, 95%, 96%, 97%, 98%, 99% or more sequence identity to SEQ ID NO: 70;
   (iv) a nucleic acid capable of hybridizing under stringent conditions to any one of the nucleic acids given in (i), (ii) or (iii) above.
12. An isolated polypeptide comprising:
   (i) an amino acid sequence having, in increasing order of preference, at least 60%, 65%, 70%, 75%, 80%, 85%, 90%, 95%, 96%, 97%, 98%, 99% or 100% sequence identity to the amino acid sequence given in SEQ ID NO: 70.
   (ii) derivatives of any of the amino acid sequences given in (I).
13. Plant or part thereof, including seeds, obtainable by a method according to any preceding item, wherein said plant or part thereof comprises a recombinant nucleic acid encoding an LBD polypeptide.
14. Construct comprising:
   (i) nucleic acid encoding an LBD polypeptide as defined in items 1, 2 or 12;
   (ii) one or more control sequences capable of driving expression of the nucleic acid sequence of (a); and optionally
   (iii) a transcription termination sequence.
15. Construct according to item 14, wherein one of said control sequences is a constitutive promoter, preferably a GOS2 promoter, most preferably a GOS2 promoter from rice.
16. Use of a construct according to item 14 or 15 in a method for making plants having increased yield, particularly increased biomass and/or increased seed yield relative to control plants.
17. Plant, plant part or plant cell transformed with a construct according to item 14 or 15.
18. Method for the production of a transgenic plant having increased yield, particularly increased biomass and/or increased seed yield relative to control plants, comprising:
   (i) introducing and expressing in a plant a nucleic acid encoding an LBD polypeptide as defined in item 1, 2 or 12; and
   (ii) cultivating the plant cell under conditions promoting plant growth and development.
19. Transgenic plant having increased yield, particularly increased biomass and/or increased seed yield, relative to control plants, resulting from increased expression of a nucleic acid encoding an LBD polypeptide as defined in item 1, 2 or 12, or a transgenic plant cell derived from said transgenic plant.
20. Transgenic plant according to item 13, 17 or 19, or a transgenic plant cell derived thereof, wherein said plant is a crop plant or a monocot or a cereal, such as rice, maize, wheat, barley, millet, rye, triticale, sorghum and oats.
21. Harvestable parts of a plant according to item 19, wherein said harvestable parts are preferably shoot biomass and/or seeds.
22. Products derived from a plant according to item 19 and/or from harvestable parts of a plant according to item 20.
23. Use of a nucleic acid encoding an LBD polypeptide in increasing yield, particularly in increasing seed yield and/or shoot biomass in plants, relative to control plants.
24. A method for enhancing yield-related traits in plants relative to control plants, comprising modulating expression in a plant of a nucleic acid encoding a JMJC polypeptide, wherein said JMJC polypeptide comprises a JmjC domain.
25. Method according to item 24 wherein said JmjC domain is represented by a sequence having in increasing order of preference at least 50%, 55%, 60%, 65%, 70%, 75%, 80%, 85%, 90%, 95%, or more sequence identity to:
   (i) SEQ ID NO: 78; and/or
   (ii) one of the JmjC domains comprised in the JMJC polypeptides represented by SEQ ID NO: 84; SEQ ID NO: 86; SEQ ID NO: 96; SEQ ID NO: 98; SEQ ID NO: 104; SEQ ID NO: 108; SEQ ID NO: 110; SEQ ID NO: 112; SEQ ID NO: 114; SEQ ID NO: 116; SEQ ID NO: 118; SEQ ID NO: 120; SEQ ID NO: 122; SEQ ID NO: 124; SEQ ID NO: 128; SEQ ID NO: 130; SEQ ID NO: 132; and SEQ ID NO: 134, whose amino acid coordinates are given in Table B4
26. Method according to item 24 and item 25, wherein said JMJC polypeptide comprising a motif having in increasing order of preference at least 50%, 55%, 60%, 65%, 70%, 75%, 80%, 85%, 90%, 95% or more sequence identity to any of:
   (i) SEQ ID NO: 79,
   (ii) SEQ ID NO: 80,
   (iii) SEQ ID NO: 81,
   (iv) SEQ ID NO: 82;
27. Method according to item 24 or 26, wherein said modulated expression is effected by introducing and expressing in a plant a nucleic acid encoding a JMJC polypeptide.
28. Method according to any one of items 24 to 27, wherein said modulating expression is an increase in the expression.
29. Method according to any one of items 24 to 28, wherein said nucleic acid encoding a JMJC polypeptide encodes any one of the proteins listed in Table B1 or is a portion of such a nucleic acid, or a nucleic acid capable of hybridising with such a nucleic acid.
30. Method according to one of items 24 to 29, wherein said nucleic acid sequence encodes an orthologue or paralogue of any of the proteins given in Table B1.
31. Method according to item 30, wherein said nucleic acid encodes SEQ ID NO: 74.
32. Method according to any one of items 24 to 31, wherein said enhanced yield-related traits comprise increased yield, preferably increased harvest index and/or seed yield relative to control plants.
33. Method according to any one of items 24 to 32, wherein said enhanced yield-related traits comprise plant early vigour relative to control plants.
34. Method according to any one of items 24 to 33, wherein said enhanced yield-related traits are obtained under non-stress conditions.
35. Method according to any one of items 24 to 33, wherein said enhanced yield-related traits are obtained under mild drought stress growth conditions.
36. Method according to any one of items 24 to 33, wherein said enhanced yield-related traits are obtained under growth conditions of nitrogen deficiency.
37. Method according to any one of items 27 to 36, wherein said nucleic acid is operably linked to a constitutive promoter, preferably to a GOS2 promoter, most preferably to a GOS2 promoter from rice.
38. Method according to any one of items 24 to 37, wherein said nucleic acid encoding a JMJC polypeptide is of plant origin, preferably from a dicotyledonous plant, further preferably from the family Brassicaceae, more preferably from the genus Arabidopsis, most preferably from Arabidopsis thaliana.
39. Plant or part thereof, including seeds, obtainable by a method according to any one of items 24 to 38, wherein said plant or part thereof comprises a recombinant nucleic acid encoding a JMJC polypeptide.
40. Construct comprising:
   (i) nucleic acid encoding a JMJC polypeptide as defined in items 24 to 26;
   (ii) one or more control sequences capable of driving expression of the nucleic acid sequence of (i); and optionally
   (iii) a transcription termination sequence.
41. Construct according to item 40, wherein one of said control sequences is a constitutive promoter, preferably a GOS2 promoter, most preferably a GOS2 promoter from rice.
42. Use of a construct according to item 40 or 41 in a method for making plants having increased yield related traits, particularly plant early vigour and/or increased seed yield relative to control plants.
43. Plant, plant part or plant cell transformed with a construct according to item 40 or 41.
44. Method for the production of a transgenic plant having increased yield related traits, particularly plant early vigour and/or increased seed yield relative to control plants, comprising:
   (i) introducing and expressing in a plant a nucleic acid encoding a JMJC polypeptide as defined in item 24 to 26; and
   (ii) cultivating the plant cell under conditions promoting plant growth and development.
45. Transgenic plant having increased yield, particularly increased plant seedling vigour and/or increased seed yield, relative to control plants, resulting from increased expression of a nucleic acid encoding a JMJC polypeptide as defined in item 24 to 26, or a transgenic plant cell derived from said transgenic plant.
46. Transgenic plant according to item 39, 43 or 45, or a transgenic plant cell derived thereof, wherein said plant is a crop plant or a monocot or a cereal, such as rice, maize, wheat, barley, millet, rye, triticale, sorghum and oats.
47. Harvestable parts of a plant according to item 46, wherein said harvestable parts are preferably shoot biomass and/or seeds.
48. Products derived from a plant according to item 46 and/or from harvestable parts of a plant according to item 47.
49. Use of a nucleic acid encoding a JMJC polypeptide in increasing yield, particularly in increasing seed yield and/or shoot biomass in plants, relative to control plants.
50. An isolated nucleic acid molecule comprising any one of the following features:
   (i) a nucleic acid represented by SEQ ID NO: 169;
   (ii) a nucleic acid or fragment thereof that is complementary to SEQ ID NO: 169;
   (iii) a nucleic acid encoding an JMJC polypeptide having, in increasing order of preference, at least 70%, 75%, 80%, 85%, 90%, 95%, 96%, 97%, 98%, 99% or more sequence identity to the amino acid sequence given in SEQ ID NO: 170;
   (iv) a nucleic acid capable of hybridizing under stringent conditions to any one of the nucleic acids given in (i), (ii) or (iii) above.
51. An isolated polypeptide molecule comprising:
   (i) an amino acid sequence having, in increasing order of preference, at least 70%, 75%, 80%, 85%, 90%, 95%, 96%, 97%, 98%, 99% or 100% or more sequence identity to the amino acid sequence given in SEQ ID NO: 170;
   (ii) derivatives of any of the amino acid sequences given in (i).
52. A method for enhancing yield-related traits in plants relative to control plants, comprising modulating expression in a plant of a nucleic acid encoding a Casein kinase I (CKI) wherein said CKI is selected from SEQ ID NO: 174 or an orthologue or paralogue thereof.
53. Method according to item 52, wherein said modulated expression is effected by introducing and expressing in a plant a nucleic acid encoding said CKI polypeptide.
54. Method according to item 52 or 53, wherein said nucleic acid encoding said CKI polypeptide is a portion of SEQ ID NO: 173, or a nucleic acid capable of hybridising with such a nucleic acid.
55. Method according to any one of items 52 to 54, wherein said nucleic acid sequence encodes SEQ ID NO: 174.
56. Method according to any one of items 52 to 55, wherein said enhanced yield-related traits comprise increased early vigour and/or increased yield, preferably increased biomass and/or increased seed yield relative to control plants.
57. Method according to any one of items 52 to 56, wherein said enhanced yield-related traits are obtained under non-stress conditions.
58. Method according to any one of items 52 to 56, wherein said enhanced yield-related traits are obtained under abiotic stress conditions.
59. Method according to item 58, wherein said abiotic stress conditions are selected from one or more of: conditions of drought stress, conditions of salt stress, and conditions of nitrogen deficiency.
60. Method according to any one of items 53 to 59, wherein said nucleic acid is operably linked to a seed-specific promoter.
61. Method according to any one of items 53 to 60, wherein said seed-specific promoter is a WSI18 promoter, preferably to a WSI18 promoter from rice.
62. Method according to any one of items 52 to 61, wherein said nucleic acid encoding a CKI polypeptide is of plant origin.
63. Method according to any one of items 52 to 62, wherein said plant origin is from preferably a dicotyledonous plant, further preferably from the family Solanaceae, more preferably from the genus Nicotiana tabacum.
64. Plant or part thereof, including seeds, obtainable by a method according to any one of items 52 to 63, wherein said plant or part thereof comprises a recombinant nucleic acid encoding a CKI polypeptide.
65. Construct comprising:
   (i) nucleic acid encoding a CKI polypeptide as defined in item 52;
   (ii) one or more control sequences capable of driving expression of the nucleic acid sequence of (a); and optionally
   (iii) a transcription termination sequence.
66. Construct according to item 65, wherein one of said control sequences is a seed specific promoter, preferably a WSI18 promoter.
67. Construct according to item 65, wherein one of said seed specific promoter is a WSI18 promoter, most preferably a WSI18 promoter from rice.
68. Use of a construct according to any of items 65 to 67 in a method for making plants having increased yield-related traits, particularly increased early vigour, increased biomass and/or increased seed yield relative to control plants.
69. Plant, plant part or plant cell transformed with a construct according to any of items 65 to 67.
70. Method for the production of a transgenic plant having increased yield, particularly increased biomass and/or increased seed yield relative to control plants, comprising:
   (i) introducing and expressing in a plant a nucleic acid encoding a CKI polypeptide as defined in item 52; and
   (ii) cultivating the plant cell under conditions promoting plant growth and development.
71. Transgenic plant having increased yield-related traits, particularly increased early vigour, increased biomass and/or increased seed yield, relative to control plants, resulting from increased expression of a nucleic acid encoding a CKI polypeptide as defined in item 52, or a transgenic plant cell derived from said transgenic plant.
72. Transgenic plant according to item 64, 69 or 71, or a transgenic plant cell derived thereof, wherein said plant is a crop plant or a monocot or a cereal, such as rice, maize, wheat, barley, millet, rye, triticale, sorghum and oats.
73. Harvestable parts of a plant according to item 72, wherein said harvestable parts are preferably shoot biomass and/or seeds.
74. Products derived from a plant according to item 72 and/or from harvestable parts of a plant according to item 73.
75. Use of a nucleic acid encoding a CKI polypeptide in increasing yield-related traits, particularly in increasing one or more of early vigour, seed yield and shoot biomass in plants, relative to control plants.
76. A method for enhancing yield-related traits, preferably enhancing seed-yield related-traits, in plants relative to control plants, comprising modulating, preferably increasing, expression in a plant of a nucleic acid sequence encoding a plant homeodomain finger-homeodomain (PHDf-HD) polypeptide, which PHDf-HD polypeptide comprises: (i) a domain having at least 25%, 30%, 35%, 40%, 45%, 50%, 55%, 60%, 65%, 70%, 75%, 80%, 85%, 90%, 95%, 98%, 99% or more amino acid sequence identity to a leucine zipper/plant homeodomain finger (ZIP/PHDf) domain as represented by SEQ ID NO: 233; and (ii) a domain having at least 25%, 30%, 35%, 40%, 45%, 50%, 55%, 60%, 65%, 70%, 75%, 80%, 85%, 90%, 95%, 98%, 99% or more amino acid sequence identity to a homeodomain (HD) as represented by SEQ ID NO: 234, and optionally selecting for plants having enhanced yield-related traits.
77. Method according to item 76, wherein said PHDf-HD polypeptide comprises: (i) a PHD domain as represented by PFAM00628; and (ii) an HD as represented by PFAM00046.
78. Method according to item 76 or 77, wherein said PHDf-HD polypeptide, when used in the construction of a HD phylogenetic tree, such as the one depicted in FIG. 13, clusters with the PHDf-HD group of polypeptides comprising the polypeptide sequence as represented by SEQ ID NO: 180, rather than with any other HD group.
79. Method according to any one of items 76 to 78, wherein said PHDf-HD polypeptide has in increasing order of preference at least 30%, 35%, 40%, 45%, 50%, 55%, 60%, 65%, 70%, 75%, 80%, 85%, 90%, 95%, 98%, 99% or more sequence identity to the PHDf-HD polypeptide as represented by SEQ ID NO: 180 or to any of the polypeptide sequences given in Table D1 herein.
80. Method according to any one of items 76 to 79, wherein said nucleic acid sequence encoding a PHDf-HD polypeptide is represented by any one of the nucleic acid sequence SEQ ID NOs given in Table D1 or a portion thereof, or a sequence capable of hybridising with any one of the nucleic acid sequences SEQ ID NOs given in Table D1.
81. Method according to any one of items 76 to 80, wherein said nucleic acid sequence encodes an orthologue or paralogue of any of the SEQ ID NOs given in Table D1.
82. Method according to any one of items 76 to 81, wherein said modulated, preferably increased, expression is effected by any one or more of: T-DNA activation tagging, TILLING, or homologous recombination.
83. Method according to any one of items 76 to 82, wherein said increased expression is effected by introducing and expressing in a plant a nucleic acid sequence encoding a PHDf-HD polypeptide.
84. Method according to any one of items 76 to 83, wherein said yield-related traits are seed yield-related traits, comprising one or more of: (i) increased number of primary panicle; (ii) increased total seed weight per plant; (iii) increased number of (filled) seeds; (iv) increased TKW; or (v) increased harvest index.
85. Method according to any one of items 76 to 84, wherein said nucleic acid sequence is operably linked to a constitutive promoter, preferably to a plant constitutive promoter, more preferably to a GOS2 promoter, most preferably to a GOS2 promoter from rice.
86. Method according to any one of items 76 to 85, wherein said nucleic acid sequence encoding a PHDf-HD polypeptide is of plant origin, preferably from a monocotyledonous plant, further preferably from the family Poacae more preferably from the genus Oryza, most preferably from Oryza sativa.
87. Plants, parts thereof (including seeds), or plant cells obtainable by a method according to any one of items 76 to 86, wherein said plant, part or cell thereof comprises an isolated nucleic acid transgene encoding a PHDf-HD polypeptide operably linked to a plant constitutive promoter.
88. Construct comprising:
   (i) A nucleic acid sequence encoding a PHDf-HD polypeptide as defined in any one of items 76 to 81;
   (ii) one or more control sequences capable of driving expression of the nucleic acid sequence of (i); and optionally
   (iii) a transcription termination sequence.
   wherein at least one of the control sequences is a plant constitutive promoter, preferably a GOS2 promoter.
89. Use of a construct according to items 87 in a method for making plants having enhanced yield-related traits relative to control plants, which enhanced yield-related traits, preferably enhanced seed yield-related traits, are one or more of: (i) increased number of primary panicle; (ii) increased total seed weight per plant; (iii) increased number of (filled) seeds; (iv) increased TKW; or (v) increased harvest index.
90. Plant, plant part or plant cell transformed with a construct according to item 87 or 88.
91. Method for the production of transgenic plants having enhanced yield-related traits relative to control plants, comprising:
   (i) introducing and expressing in a plant, plant part, or plant cell, a nucleic acid sequence encoding a PHDf-HD polypeptide as defined in any one of items 76 to 81, under the control of plant constitutive promoter; and
   (ii) cultivating the plant cell under conditions promoting plant growth and development.
92. Transgenic plant having enhanced yield-related traits, preferably enhanced seed yield-related traits, relative to control plants, resulting from modulated, preferably increased, expression of a nucleic acid sequence encoding a PHDf-HD polypeptide as defined in any one of items 76 to 81, operably linked to a plant constitutive promoter, or a transgenic plant cell derived from said transgenic plant.
93. Transgenic plant according to item 87, 90 or 92, wherein said plant is a crop plant or a monocot or a cereal, such as rice, maize, wheat, barley, millet, rye, triticale, sorghum and oats, or a transgenic plant cell derived from said transgenic plant.
94. Harvestable parts comprising a nucleic acid sequence encoding a PHDf-HD polypeptide of a plant according to item 93, wherein said harvestable parts are preferably seeds.
95. Products derived from a plant according to item 93 and/or from harvestable parts of a plant according to item 94.
96. Use of a nucleic acid sequence encoding a PHDf-HD polypeptide as defined in any one of items 76 to 81 in enhancing yield-related traits in plants, preferably in enhancing seed yield-related traits, comprising one or more of: (i) increased number of primary panicles; (ii) increased total seed weight per plant; (iii) increased number of (filled) seeds; (iv) increased TKW; or (v) increased harvest index.
97. An isolated nucleic acid molecule comprising any one of the following features:
   (i) a nucleic acid represented by SEQ ID NO: 242;
   (ii) a nucleic acid or fragment thereof that is complementary to SEQ ID NO: 242;
   (iii) a nucleic acid encoding an JMJC polypeptide having, in increasing order of preference, at least 70%, 75%, 80%, 85%, 90%, 95%, 96%, 97%, 98%, 99% or more sequence identity to the amino acid sequence given in SEQ ID NO: 243;
   (iv) a nucleic acid capable of hybridizing under stringent conditions to any one of the nucleic acids given in (i), (ii) or (iii) above.
98. An isolated polypeptide molecule comprising:
   (i) an amino acid sequence having, in increasing order of preference, at least 70%, 75%, 80%, 85%, 90%, 95%, 96%, 97%, 98%, 99% or 100% or more sequence identity to the amino acid sequence given in SEQ ID NO: 243;
   (ii) derivatives of any of the amino acid sequences given in (i).
99. A method for enhancing yield-related traits in plants relative to control plants, comprising modulating expression in a plant of a nucleic acid encoding a bHLH11-like polypeptide, wherein said bHLH-11 like polypeptide comprises a Helix-Loop-Helix domain.
100. Method according to item 99, wherein said bHLH11-like polypeptide comprises one or more of the following motifs:
   (i) Motif 1 (SEQ ID NO: 246);
   (ii) Motif 2 (SEQ ID NO: 247);
   (iii) Motif 3 (SEQ ID NO: 248);
   (iv) Motif 4 (SEQ ID NO: 249);
   (v) Motif 5 (SEQ ID NO: 250);
   (vi) Motif 6 (SEQ ID NO: 251);
   (vii) Motif 7 (SEQ ID NO: 252).
101. Method according to item 99 or 100, wherein said modulated expression is effected by introducing and expressing in a plant a nucleic acid encoding a bHLH11-like polypeptide.
102. Method according to any one of items 99 to 101, wherein said nucleic acid encoding a bHLH11-like polypeptide encodes any one of the proteins listed in Table E1 or is a portion of such a nucleic acid, or a nucleic acid capable of hybridising with such a nucleic acid.
103. Method according to any one of items 99 to 102, wherein said nucleic acid sequence encodes an orthologue or paralogue of any of the proteins given in Table E1.
104. Method according to any one of items 99 to 103, wherein said enhanced yield-related traits comprise increased yield, preferably increased seed yield relative to control plants.
105. Method according to any one of items 99 to 104, wherein said enhanced yield-related traits are obtained under non-stress conditions.
106. Method according to any one of items 101 to 105, wherein said nucleic acid is operably linked to a constitutive promoter, preferably to a GOS2 promoter, most preferably to a GOS2 promoter from rice.
107. Method according to any one of items 99 to 106, wherein said nucleic acid encoding a bHLH11-like polypeptide is of plant origin, preferably from a monocotyledonous plant, further preferably from the family Poaceae, more preferably from the genus Triticum, most preferably from Triticum aestivum.
108. Plant or part thereof, including seeds, obtainable by a method according to any one of items 99 to 107, wherein said plant or part thereof comprises a recombinant nucleic acid encoding a bHLH11-like polypeptide.
109. Construct comprising:
   (i) nucleic acid encoding a bHLH11-like polypeptide as defined in items 99 or 100;
   (ii) one or more control sequences capable of driving expression of the nucleic acid sequence of (i); and optionally
   (iii) a transcription termination sequence.
110. Construct according to item 109, wherein one of said control sequences is a constitutive promoter, preferably a GOS2 promoter, most preferably a GOS2 promoter from rice.
111. Use of a construct according to item 109 or 110 in a method for making plants having increased yield, particularly increased seed yield relative to control plants.
112. Plant, plant part or plant cell transformed with a construct according to item 109 or 110.
113. Method for the production of a transgenic plant having increased yield, particularly increased seed yield relative to control plants, comprising:
   (i) introducing and expressing in a plant a nucleic acid encoding a bHLH11-like polypeptide as defined in item 99 or 100; and
   (ii) cultivating the plant cell under conditions promoting plant growth and development.
114. Transgenic plant having increased yield, particularly increased seed yield, relative to control plants, resulting from modulated expression of a nucleic acid encoding a bHLH11-like polypeptide as defined in item 99 or 100, or a transgenic plant cell derived from said transgenic plant.
115. Transgenic plant according to item 108, 112 or 114, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
116. Harvestable parts of a plant according to item 115, wherein said harvestable parts are preferably seeds.
117. Products derived from a plant according to item 115 and/or from harvestable parts of a plant according to item 116.
118. Use of a nucleic acid encoding a bHLH11-like polypeptide in increasing yield, particularly in increasing seed yield in plants, relative to control plants.
119. A method for enhancing yield-related traits in plants relative to control plants, comprising modulating expression in a plant of a nucleic acid encoding an ASR, wherein said ASR is represented by SEQ ID NO: 397 or an orthologue or paralogue thereof.
120. Method according to item 119, wherein said modulated expression is effected by introducing and expressing in a plant a nucleic acid encoding said ASR polypeptide.
121. Method according to item 119 or 120, wherein said nucleic acid encoding said ASR polypeptide is a portion of SEQ ID NO: 396, or a nucleic acid capable of hybridising with such a nucleic acid.
122. Method according to any one of items 119 to 121, wherein said nucleic acid sequence encodes SEQ ID NO: 397, or an orthologue or paralogue thereof.
123. Method according to any one of items 119 to 122, wherein said enhanced yield-related traits comprise increased yield, preferably increased seed yield relative to control plants.
124. Method according to any one of items 119 to 123, wherein said enhanced yield-related traits are obtained under non-stress conditions.
125. Method according to any one of items 120 to 124, wherein said nucleic acid is operably linked to a constitutive promoter, preferably to a GOS2 promoter, most preferably to a GOS2 promoter from rice.
126. Method according to any one of items 119 to 125, wherein said nucleic acid encoding an ASR polypeptide is of plant origin, preferably from a dicotyledonous plant, further preferably from the family Poaceae, more preferably from the genus Oryza, most preferably from Oryza sativa.
127. Plant or part thereof, including seeds, obtainable by a method according to any one of items 119 to 126, wherein said plant or part thereof comprises a recombinant nucleic acid encoding an ASR polypeptide.
128. Construct comprising:
   (i) nucleic acid encoding an ASR polypeptide as defined in item 119 or any one of SEQ ID NO: 401, 403, 405, 407, 409, 411, 413, 415 and 417;
   (ii) one or more control sequences capable of driving expression of the nucleic acid sequence of (a); and optionally
   (iii) a transcription termination sequence.
129. Construct according to item 128, wherein one of said control sequences is a constitutive promoter, preferably a GOS2 promoter, most preferably a GOS2 promoter from rice.
130. Use of a construct according to any of items 128 or 129 in a method for making plants having increased yield-related traits, particularly increased seed yield relative to control plants.
131. Plant, plant part or plant cell transformed with a construct according to any of items 128 or 129.
132. Method for the production of a transgenic plant having increased yield, particularly increased biomass and/or increased seed yield relative to control plants, comprising:
   (i) introducing and expressing in a plant a nucleic acid encoding an ASR polypeptide as defined in item 119; and
   (ii) cultivating the plant cell under conditions promoting plant growth and development.
133. Transgenic plant having increased yield-related traits, particularly increased seed yield, relative to control plants, resulting from increased expression of a nucleic acid encoding an ASR polypeptide as defined in item 119, or a transgenic plant cell derived from said transgenic plant.
134. Transgenic plant according to item 127, 131 or 133, or a transgenic plant cell derived thereof, wherein said plant is a crop plant or a monocot or a cereal, such as rice, maize, wheat, barley, millet, rye, triticale, sorghum and oats.
135. Harvestable parts of a plant according to item 134, wherein said harvestable parts are preferably seeds.
136. Products derived from a plant according to item 134 and/or from harvestable parts of a plant according to item 135.
137. Use of a nucleic acid encoding an ASR polypeptide in increasing yield-related traits, particularly in increasing seed yield in plants, relative to control plants.
138. An isolated nucleic acid molecule comprising any one of the following features:
   (i) a nucleic acid represented by any one of SEQ ID NO: 401, 403, 405, 407, 409, 411, 413, 415 and 417;
   (ii) the complement of a nucleic acid represented by any one of SEQ ID NO: 401, 403, 405, 407, 409, 411, 413, 415 and 417;
   (iii) a nucleic acid encoding an ASR polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by any one of SEQ ID NO: 402, 404, 406, 408, 410, 412, 414, 416 and 418.
139. An isolated polypeptide comprising:
   (i) an amino acid sequence represented by any one of SEQ ID NO: 402, 404, 406, 408, 410, 412, 414, 416 and 418;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented any one of SEQ ID NO: 402, 404, 406, 408, 410, 412, 414, 416 and 418.
   (iii) derivatives of any of the amino acid sequences given in (i) or (ii) above.
140. A method for enhancing yield-related traits in plants relative to control plants, comprising modulating expression in a plant of a nucleic acid encoding a SPL11 polypeptide, wherein said SPL11 polypeptide comprises a SBP domain having in increasing order of preference at least 70%, 75%, 80%, 85%, 90%, 95%, 97% or more sequence identity to any one of SEQ ID NO: 456 to SEQ ID NO: 468 and SEQ ID NO: 478.
141. Method according to item 140 wherein said SPL11 polypeptide in addition to the SBP domain comprises any one or more of the following conserved motifs:
   (i) Motif 1 as represented by SEQ ID NO: 469 wherein any conservative amino acid substitution and/or 1 or 2 non conservative substitution are allowed;
   (ii) Motif 2 as represented by SEQ ID NO: 470 wherein any change is allowed, provided that at least 4 amino acids have a polar side chain, preferably serine or threonine, and provided that the domain is located at the N-terminal end of the SBP domain;
   (iii) Motif 3 as represented by SEQ ID: 471 wherein 1 or 2 mismatches are allowed;
   (iv) Motif 4 as represented by SEQ ID: 472 wherein 1, 2 or 3 mismatches are allowed.
142. Method according to item 140 or 141, wherein said modulated expression is effected by introducing and expressing in a plant a nucleic acid encoding a SPL11 polypeptide as defined in item 140 or 141.
143. Method according to any of items 140 to 142, wherein said modulated expression is increased expression.
144. Method according to any of items 140 to 143, wherein said nucleic acid encoding a SPL11 polypeptide encodes any one of the proteins listed in Table 01 or is a portion of such a nucleic acid, or a nucleic acid capable of hybridising with such a nucleic acid.
145. Method according to any of items 140 to 144, wherein said nucleic acid sequence encodes an orthologue or paralogue of any of the proteins given in Table G1.
146. Method according to item 145, wherein said nucleic acid encodes SEQ ID NO: 428.
147. Method according to any of items 140 to 146, wherein said enhanced yield-related traits comprise increased yield, preferably increased total seed weight, number of filled seeds, number of seeds or florets per panicle, thousand-kernel weight, seed filling rate, and/or harvest index relative to control plants.
148. Method according to any of items 140 to 147, wherein said enhanced yield-related traits comprise plant (seedling) early vigour relative to control plants.
149. Method according to any one of items 140 to 147, wherein said enhanced yield-related traits are obtained under non-stress conditions.
150. Method according to any one of items 140 to 147, wherein said enhanced yield-related traits are obtained under mild drought stress growth conditions.
151. Method according to any one of items 140 to 147, wherein said enhanced yield-related traits are obtained under growth conditions of nitrogen deficiency.
152. Method according to any one of items 140 to 151, wherein said nucleic acid is operably linked to a constitutive promoter, preferably to a GOS2 promoter, most preferably to a GOS2 promoter from rice.
153. Method according to any one of items 140 to 151, wherein said nucleic acid is operably linked to a seed specific promoter, preferably to a WSI18 promoter, most preferably to a WSI18 promoter from rice.
154 Method according to any of items 140 to 153, wherein said nucleic acid encoding a SPL11 polypeptide is of plant origin, preferably from a dicotyledonous plant, further preferably from the family Brassicaceae, more preferably from the genus Arabidopsis, most preferably from Arabidopsis thaliana.
155. Plant or part thereof, including seeds, obtainable by a method according to any one of items 140 to 154, wherein said plant or part thereof comprises a recombinant nucleic acid encoding a SPL11 polypeptide.
156. An isolated nucleic acid molecule comprising:
   (i) a nucleic acid represented by SEQ ID NO: 448;
   (ii) the complement of a nucleic acid represented by SEQ ID NO: 448;
   (iii) a nucleic acid encoding a SPL11 polypeptide having, in increasing order of preference, at least 70%, 75%, 80%, 85%, 90%, 95%, 96%, 97%, 98%, 99% or more sequence identity to the amino acid sequence represented by SEQ ID NO: 449, and having in increasing order of preference at least 70%, 75%, 80%, 85%, 90%, 95%, 96%, 97%, 98%, 99% or more sequence identity to SEQ ID NO: 465: SYCQVEGCRTDLSSAKDYHRKHRVCEPHSKAPKVVVAGLERRFCQQCSRFHG LAEFDQKKKSCRRRLNDHNARRRKPQPEAL;
   (iv) a nucleic acid hybridising under stringent conditions to SEQ ID NO: 448.
157. An isolated polypeptide comprising:
   (i) an amino acid sequence represented by SEQ ID NO: 449;
   (ii) an amino acid sequence having, in increasing order of preference, at least 70%, 75%, 80%, 85%, 90%, 95%, 96%, 97%, 98%, 99% or more sequence identity to the amino acid sequence represented by SEQ ID NO: 449, and having in increasing order of preference at least 70%, 75%, 80%, 85%, 90%, 95%, 96%, 97%, 98%, 99% or more sequence identity to SEQ ID NO: 465:

SYCQVEGCRTDLSSAKDYHRKHRVCEPHSKAPKVVVAGLERRFCQQCSRF
HGLAEFDQKKKSCRRRLNDHNARRRKPQPEAL.

(iii) derivatives of any of the amino acid sequences given in (i) or (ii) above.
158. Construct comprising:
   (i) nucleic acid encoding a SPL11 polypeptide;
   (ii) one or more control sequences capable of driving expression of the nucleic acid sequence of (i); and optionally
   (iii) a transcription termination sequence
159. Construct according to item 158 wherein said nucleic acid encoding a SPL11 polypeptide is a nucleic acid according to item 156.
160. Construct according to item 158 or 159 wherein one of said control sequences is a constitutive promoter, preferably a GOS2 promoter, most preferably a GOS2 promoter from rice.
161. Construct according to item 158 or 159 wherein one of said control sequences is a seed specific promoter, preferably a WSI18 promoter, most preferably a WSI18 promoter from rice.
162. Use of a construct according to items 158 to 161 in a method for making plants having increased yield related traits; particularly increased seed yield relative to control plants and/or plant or seedling early vigour.
163. Plant, plant part or plant cell transformed with a construct according to items 158 to 161.
164. Method for the production of a transgenic plant having increased yield related traits, particularly increased seed yield and/or plant or seedling early vigour relative to control plants, comprising:
   (i) introducing and expressing in a plant a nucleic acid encoding a SPL11 polypeptide; and
   (ii) cultivating the plant cell under conditions promoting plant growth and development.
165. Transgenic plant having increased yield, particularly increased plant seedling vigour and/or increased seed yield, relative to control plants, resulting from increased expression of a nucleic acid encoding a SPL11 polypeptide, or a transgenic plant cell derived from said transgenic plant.
166. Transgenic plant according to item 155, 163 or 165, or a transgenic plant cell derived thereof, wherein said plant is a dicot crop plant such as soybean, cotton or canola or a monocot crop plant or a cereal, such as rice, maize, wheat, barley, millet, rye, triticale, sorghum and oats.
167. Harvestable parts of a plant according to item 166, wherein said harvestable parts are preferably shoot biomass, flowers and/or seeds.
168. Products derived from a plant according to item 166 and/or from harvestable parts of a plant according to item 167.
169. Use of a nucleic acid encoding a SPL11 polypeptide in increasing yield, particularly in increasing seed yield and/or shoot biomass in plants, relative to control plants.

### DESCRIPTION OF FIGURES

The present invention will now be described with reference to the following figures in which:

### I. LOB-Domain Comprising Protein (LOB: Lateral Organ Boundaries)

FIG. 1 represents the sequence of SEQ ID NO: 2, with the DUF260 domain shown in bold, the conserved motifs 1, 2 and 3 are underlined and the conserved Cys residues (motif of SEQ ID NO: 9) are shown in italics.

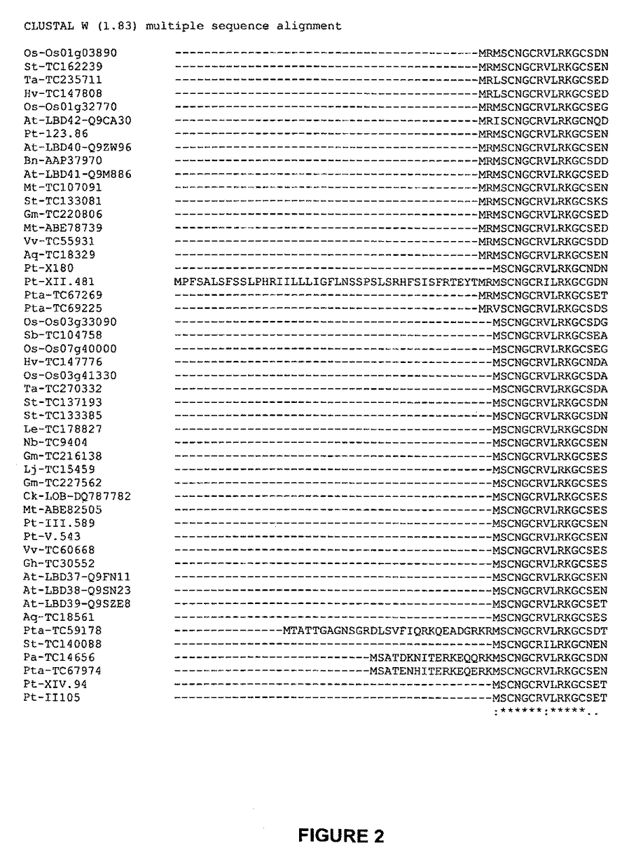

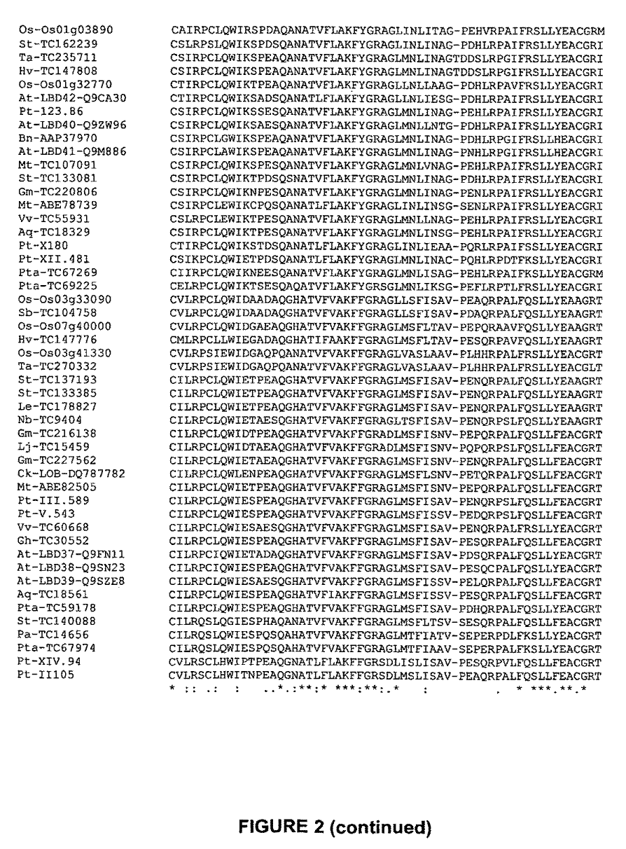

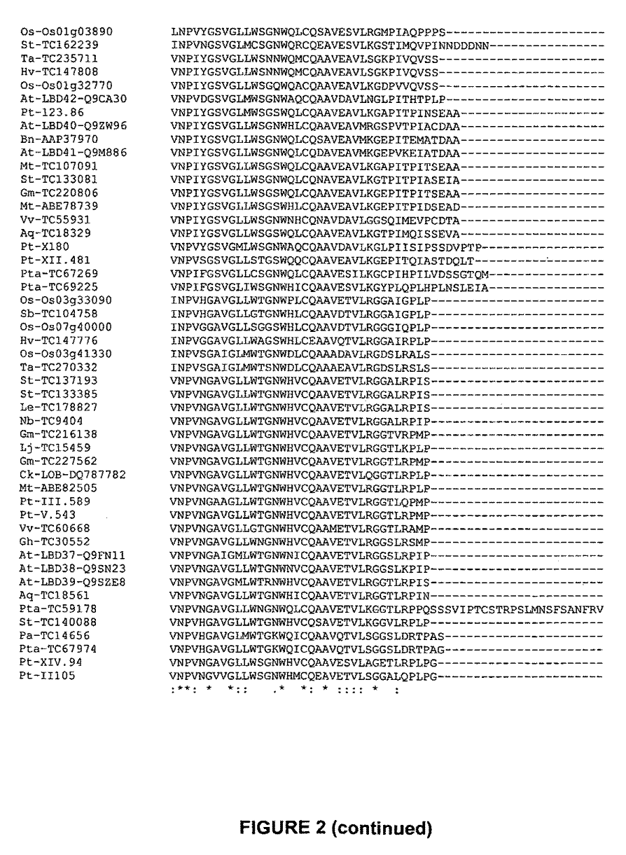

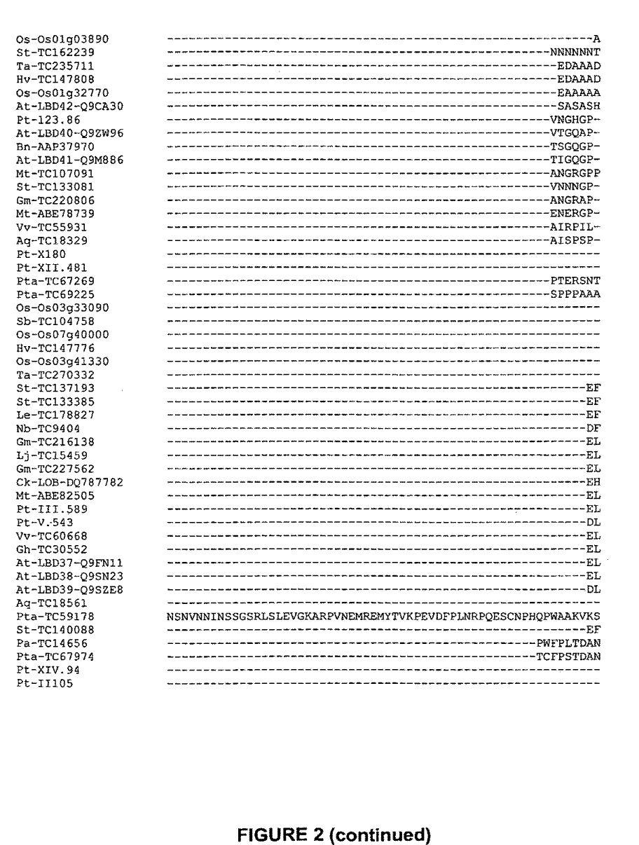

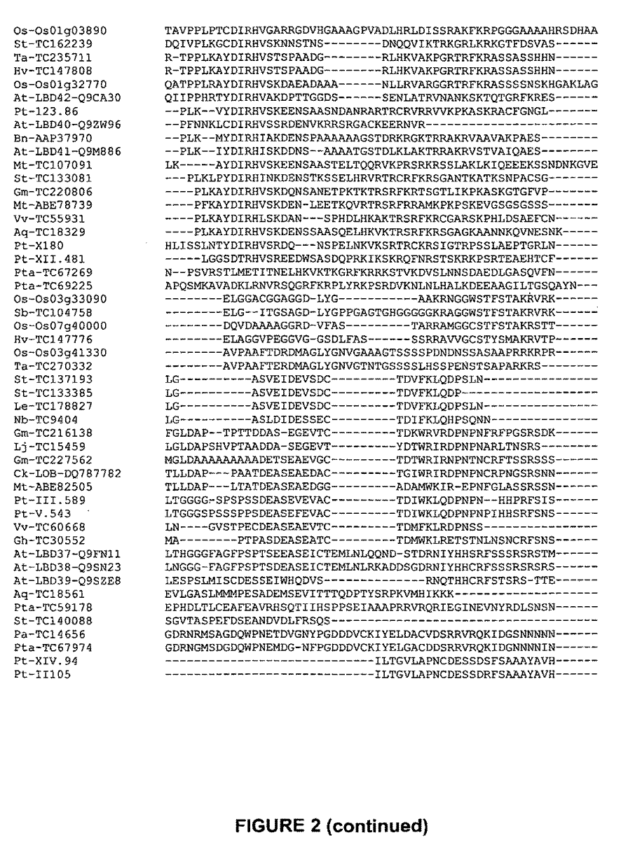

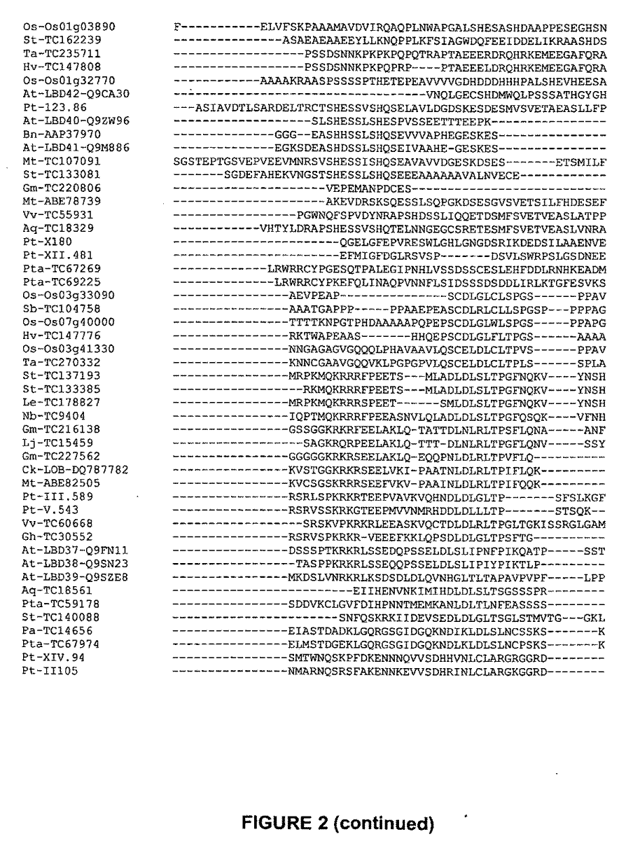

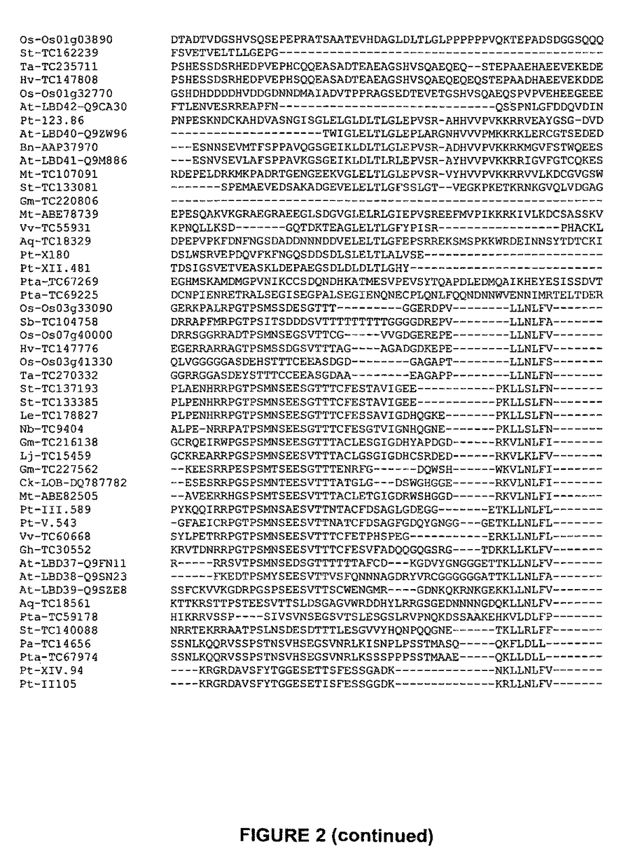

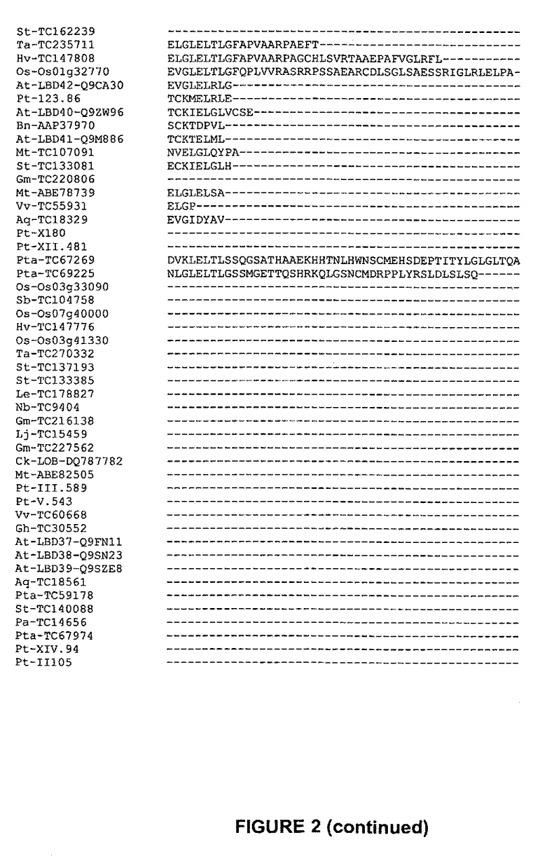

FIG. 2 represents a multiple alignment of sequences of various LBD proteins useful in the methods of the present invention.

FIG. 3 shows a phylogenetic tree of class II and class I LBD proteins. The sequence of SEQ ID NO: 2 is represented by AtLBD37.

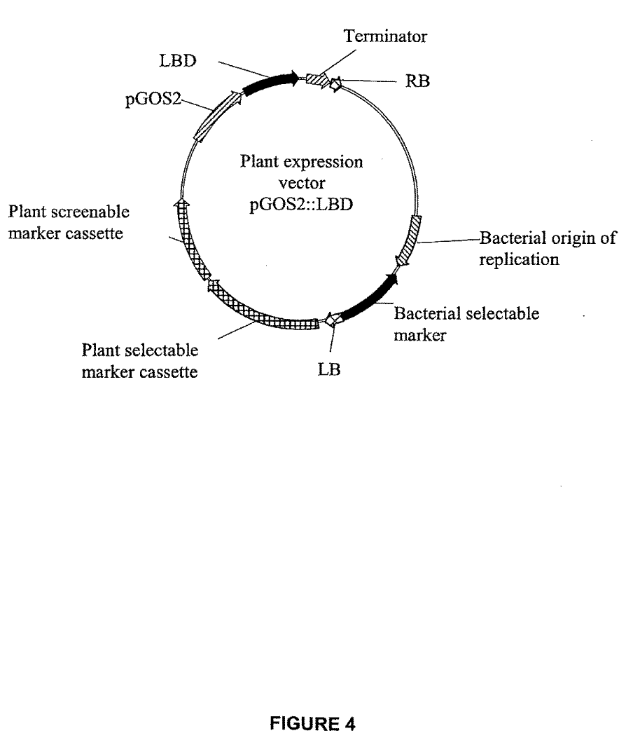

FIG. 4 represents the binary vector for increased expression in Oryza sativa of a LBD-encoding nucleic acid under the control of a rice GOS2 promoter (pGOS2)

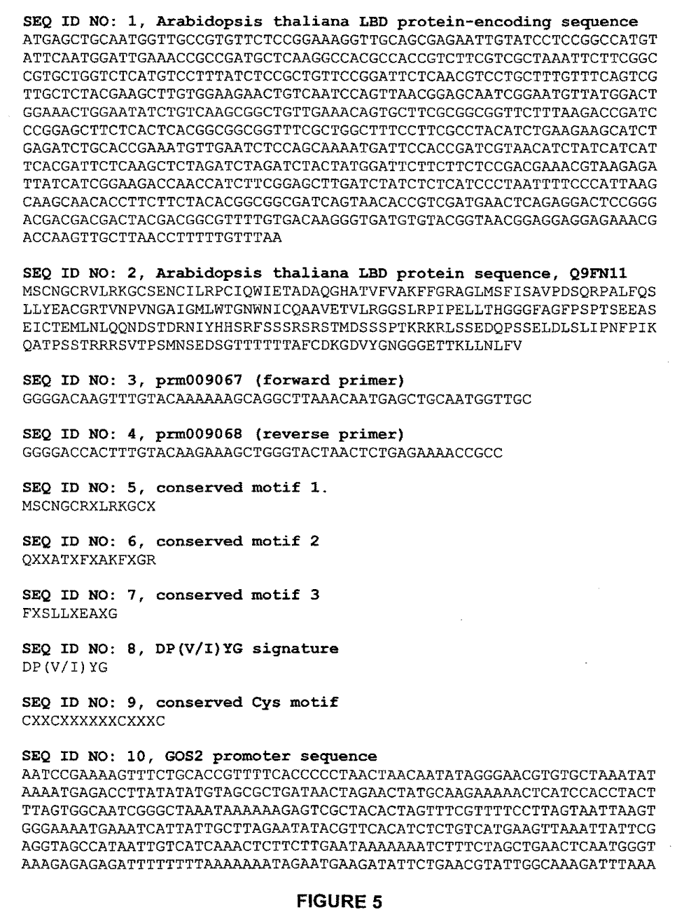

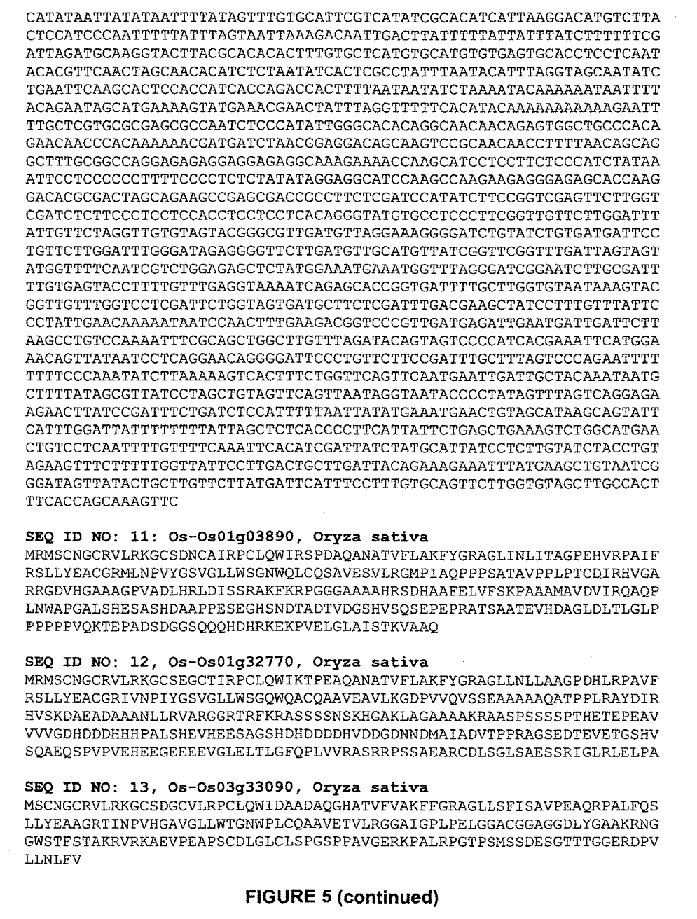

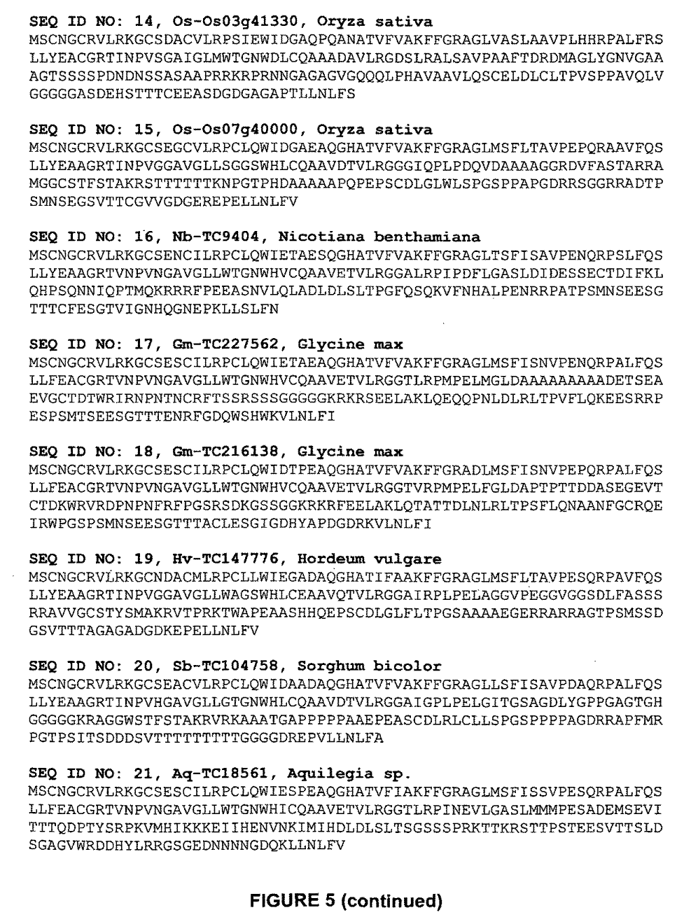

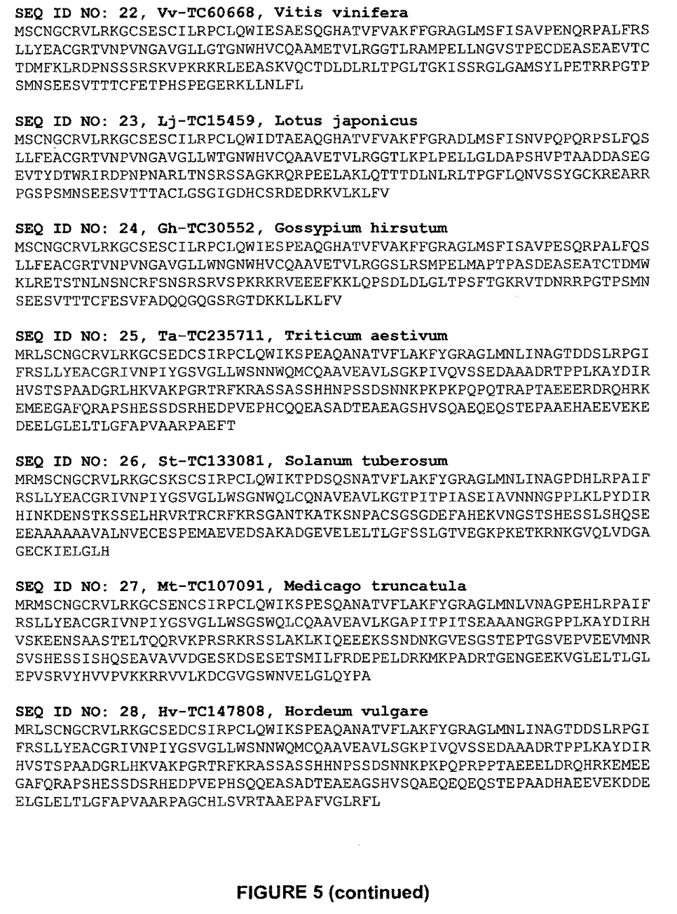

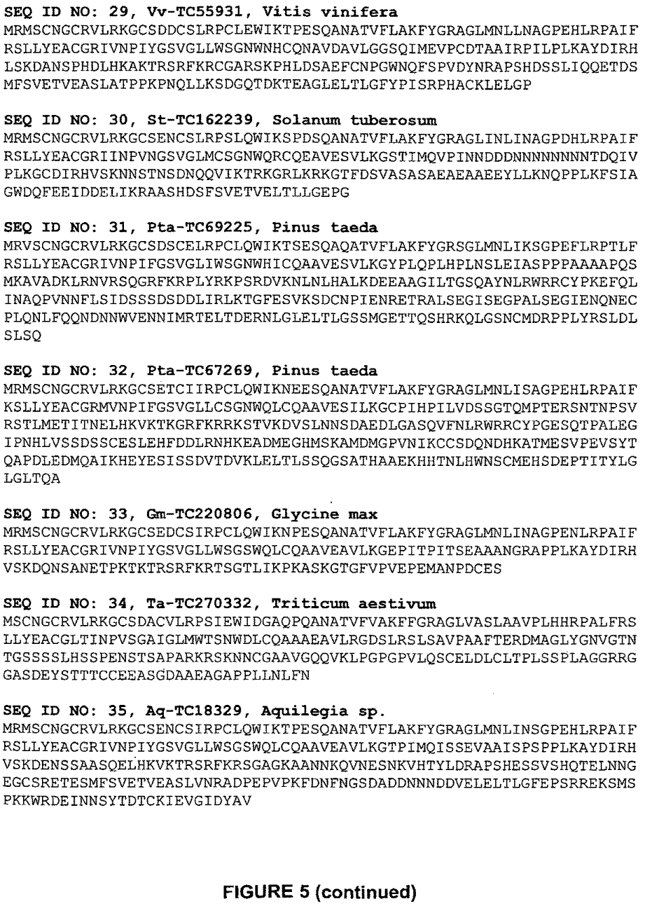

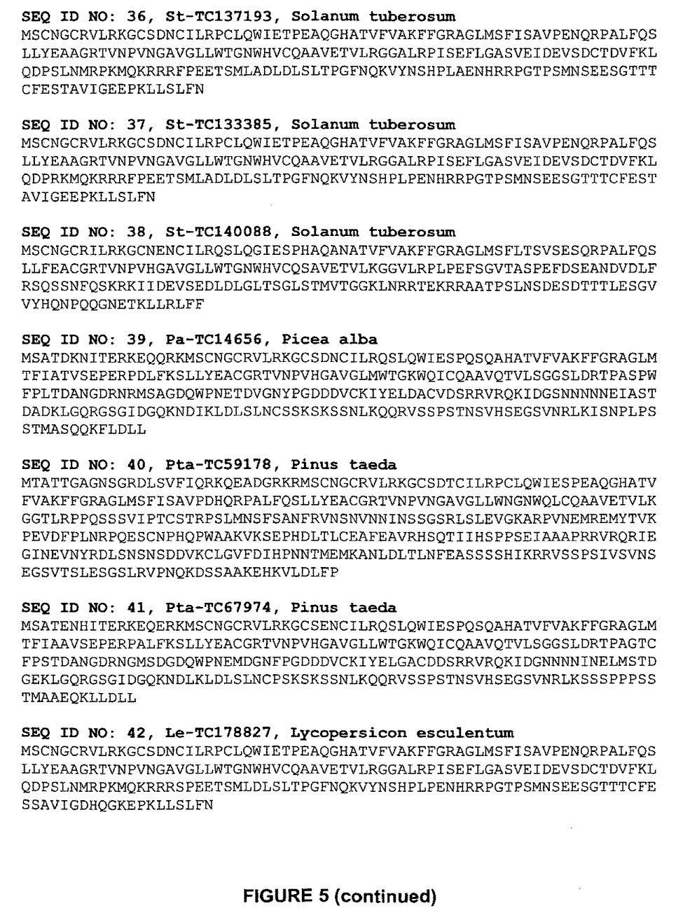

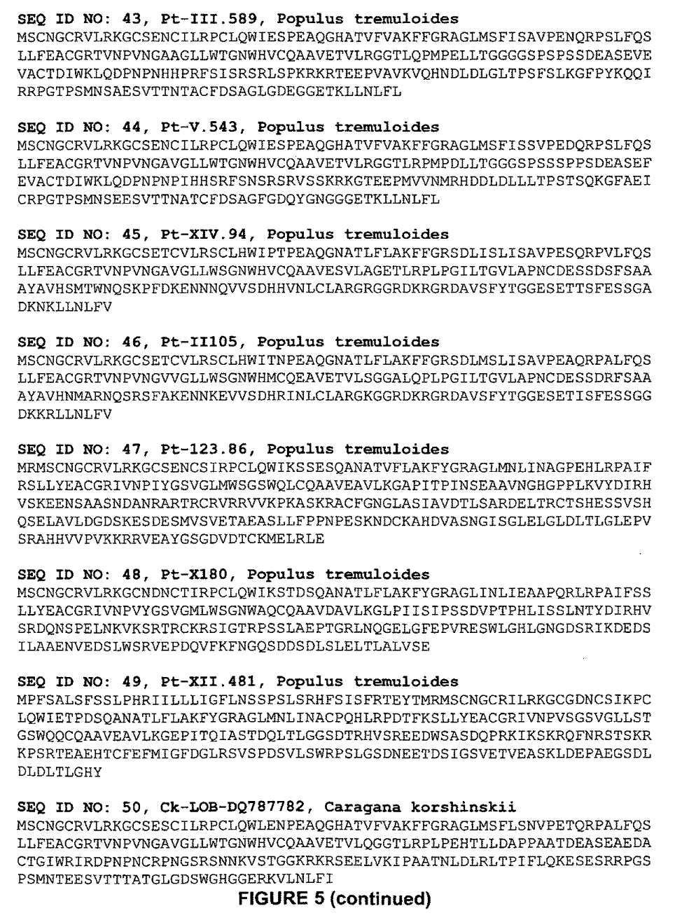

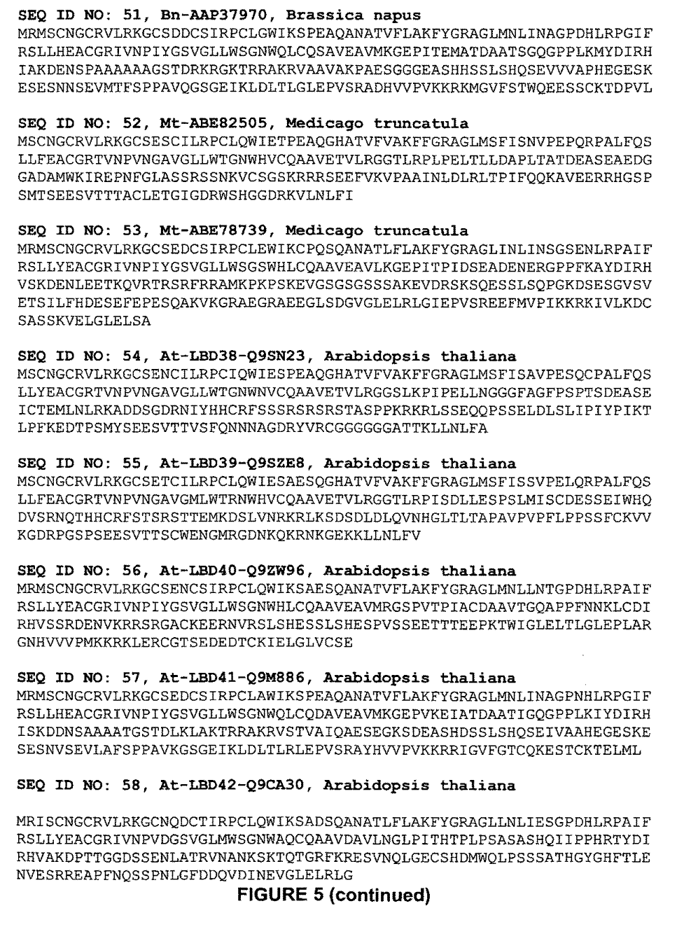

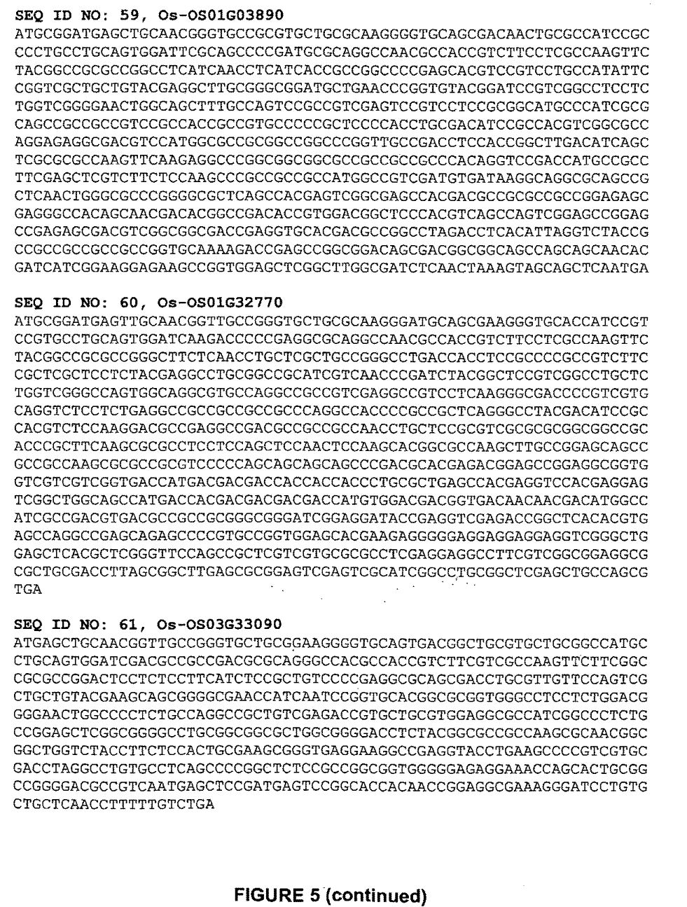

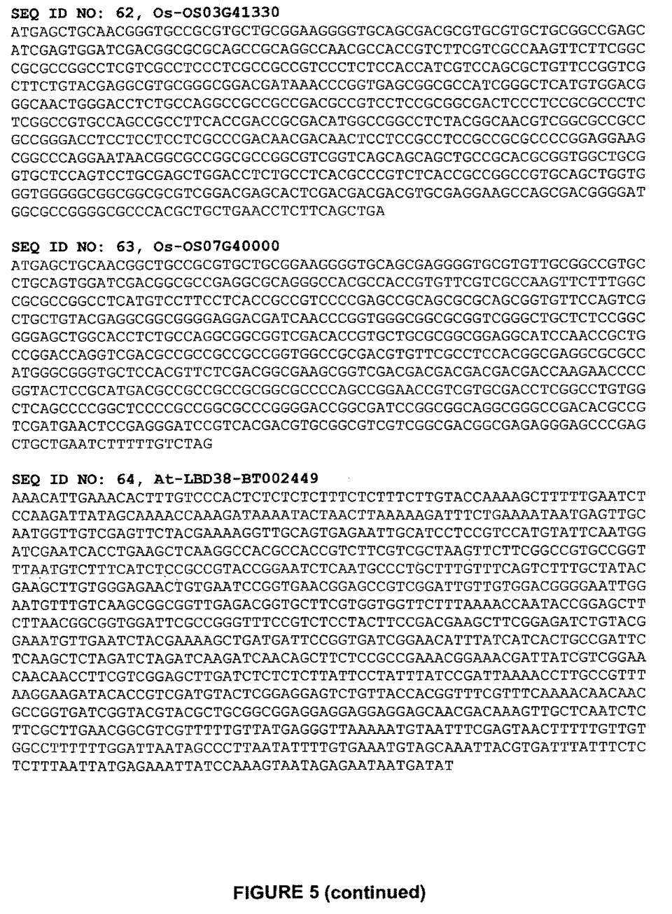

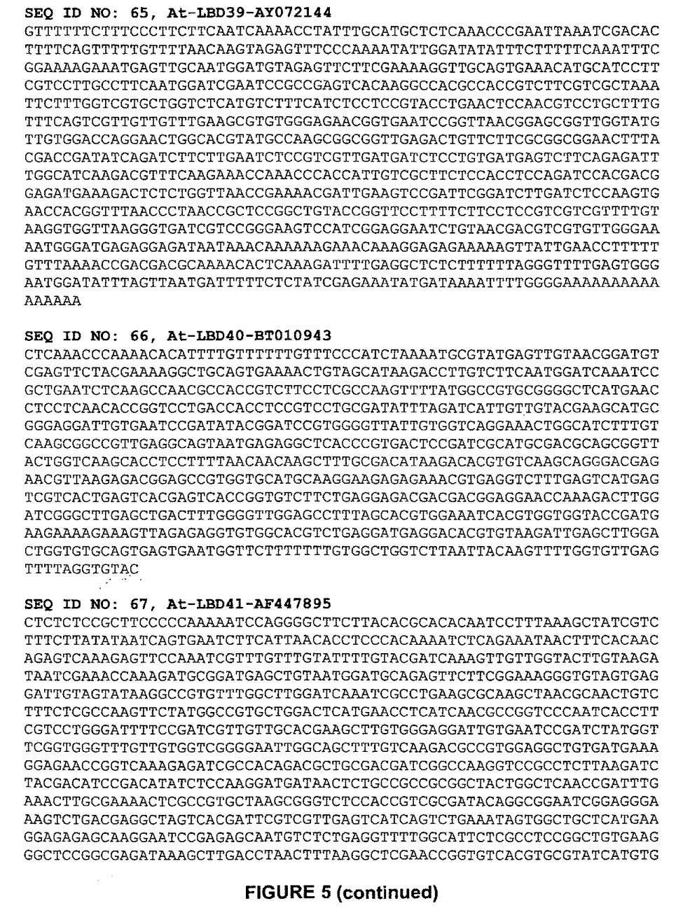

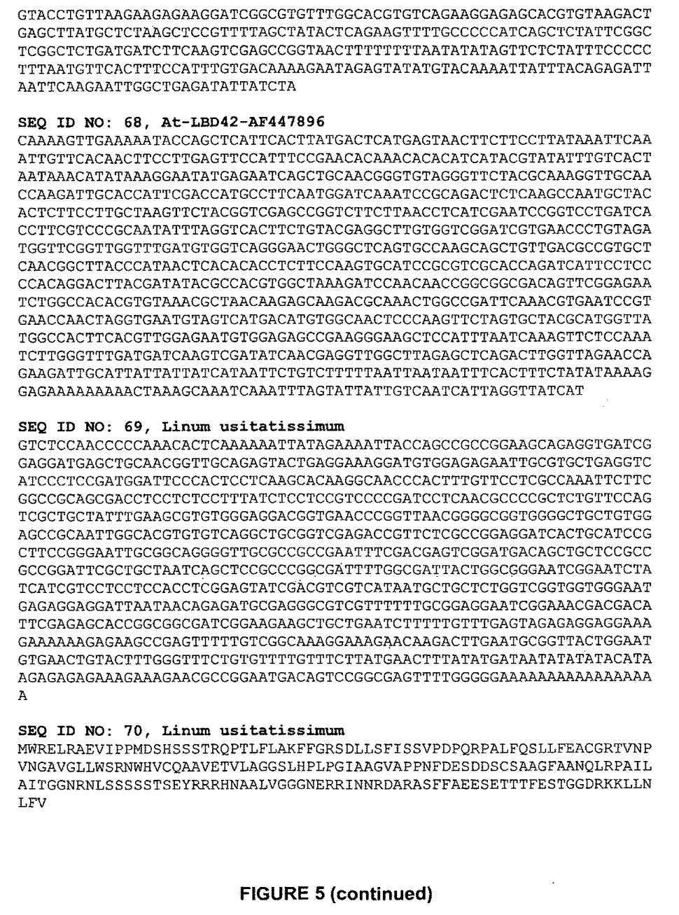

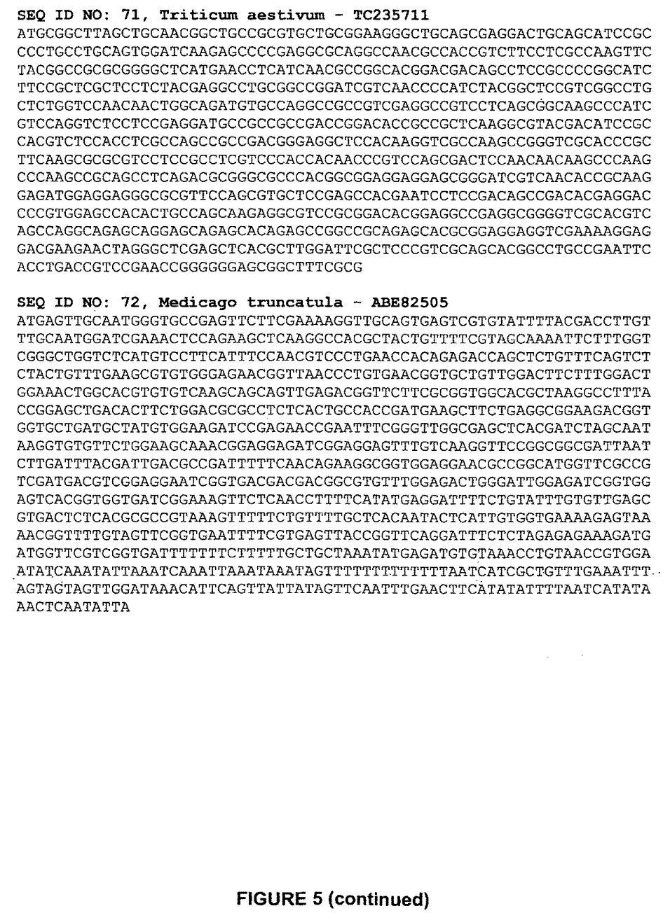

FIG. 5 details examples of LBD sequences useful in performing the methods according to the present invention.

### II. JMJC (JUMONJI-C) Polypeptide

FIG. 6 represents the amino acid sequence and domain structure of SEQ ID NO: 74. Conserved motifs and domains are indicated. JmjC domain is indicated in bold. Conserved motifs SEQ ID NO: 79, SEQ ID NO: 80 and SEQ ID NO: 81 are indicated by single, double and triple lined rectangles respectively. Underlined are the T (Theonine) and K (Lysine) amino acid residues that typically participate in 2-Oxogutarate coordination. Capital H indicates the Histidine amino acid residue coordinating the iron ion.

FIG. 7 represents a multiple alignment of JMJC proteins. The origin of the protein is indicated by the first two alphanumeric digits in the name, At: Arabidopsis thaliana, Pp: Populus trichocarpa, Os: Oryza sativa, Ot: streococcus tauri, Ce: Caenorhabditis elegans, Hs: Homo sapiens. Position of the conserved amino acid residues and domains corresponding to those described in FIG. 6 is indicated. A consensus sequence is given. Highly conserved amino acids in the consensus sequences are given; empty blank spaces represent any amino acid.

FIG. 8 shows a phylogenetic tree of JMJC polypeptides. The arrow shows SEQ ID NO: 74. Other JMJC polypeptides are named using the Genbank accession number. Group I (G I) comprises proteins of plant origin; Group II (GII) comprises proteins of non-plant origin.

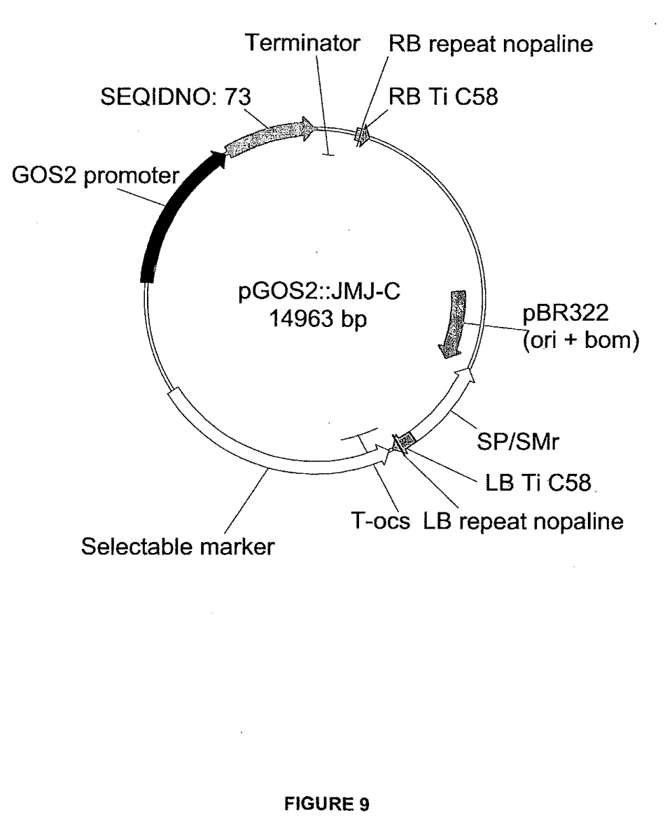

FIG. 9 represents the binary vector for increased expression in Oryza sativa of SEQ ID NO: 73 under the control of a rice GOS2 promoter (pGOS2).

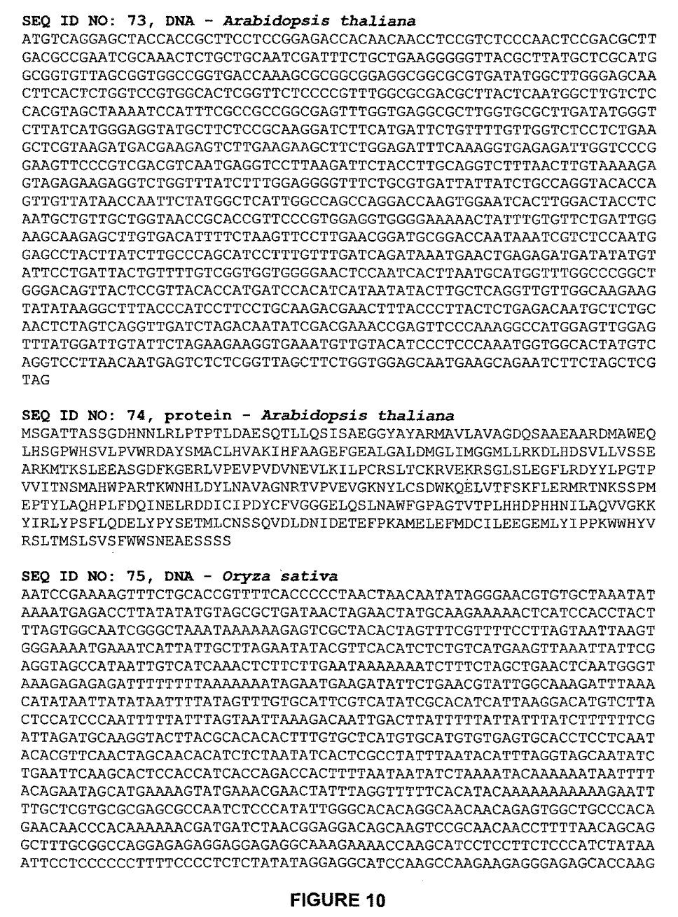

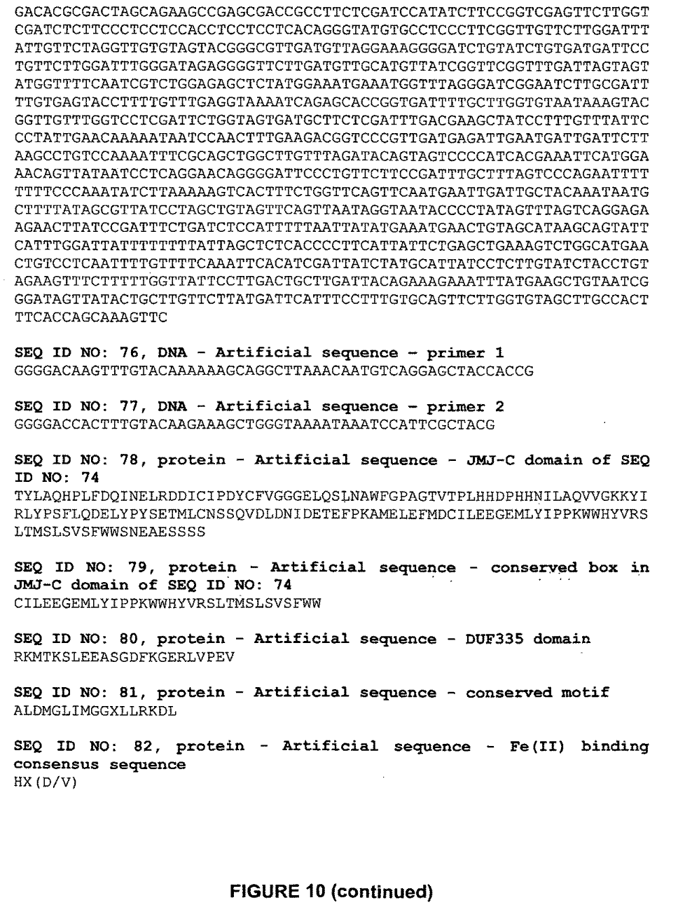

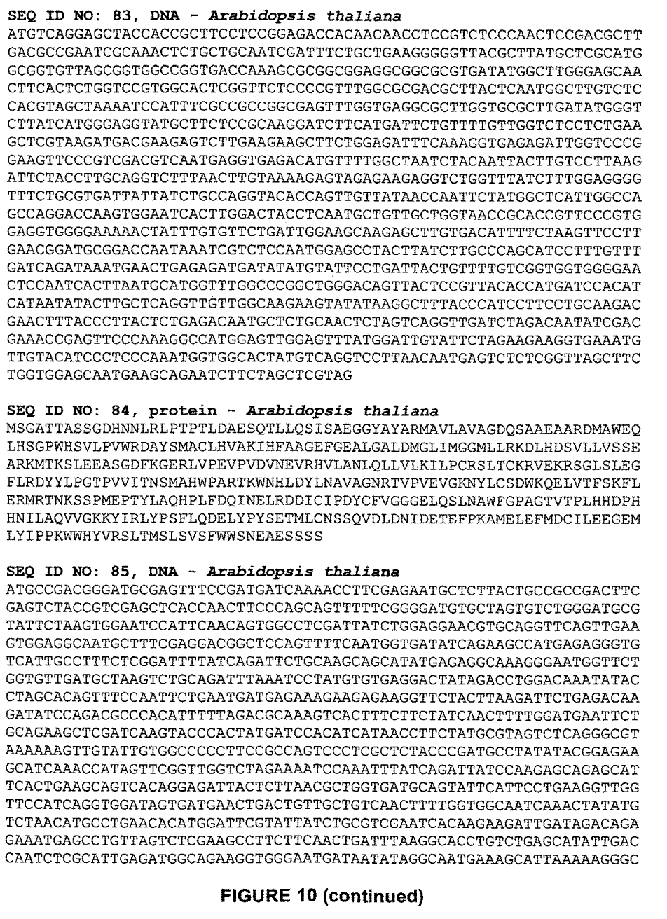

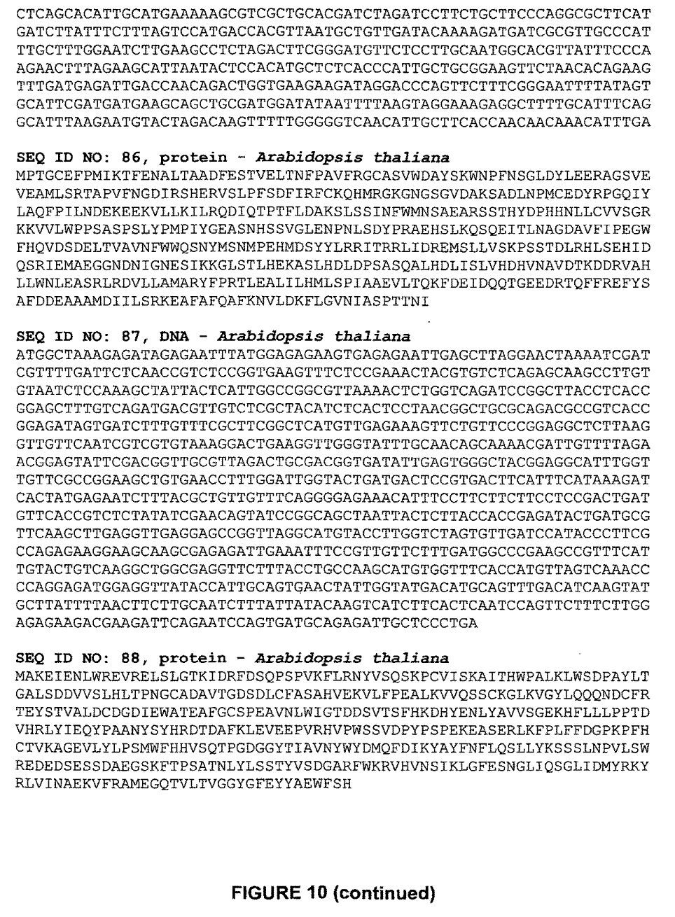

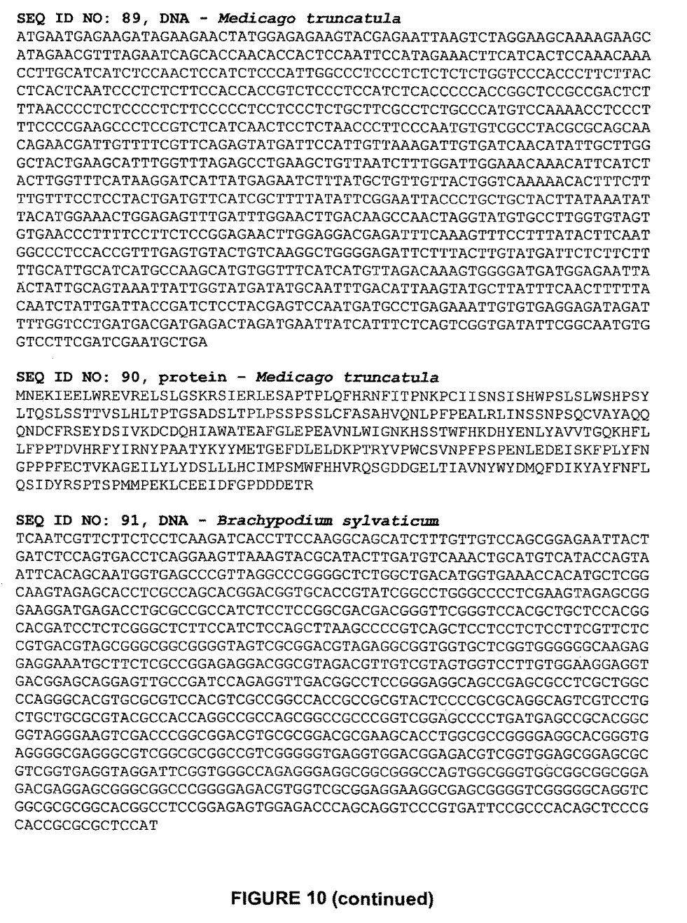

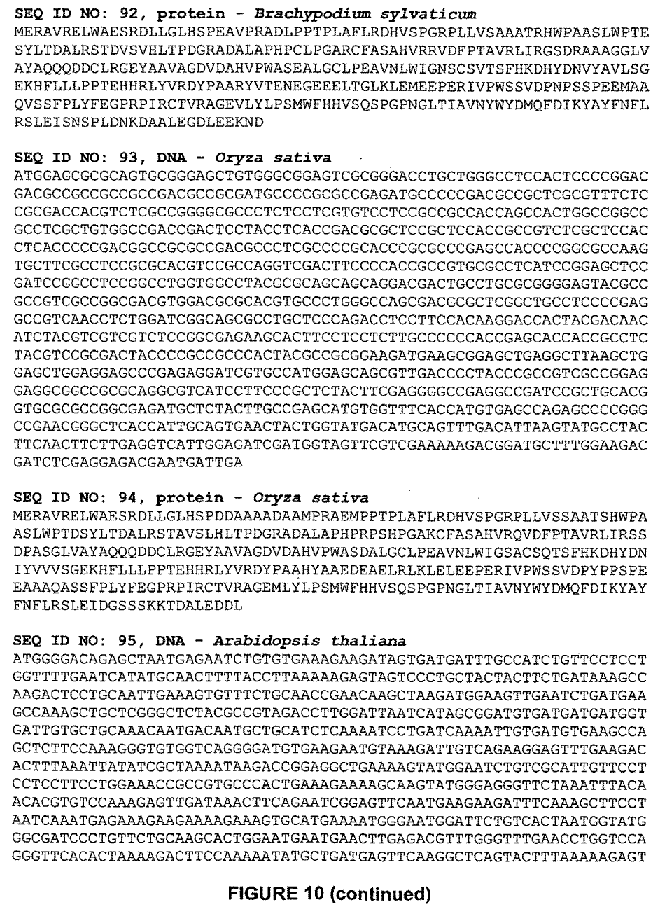

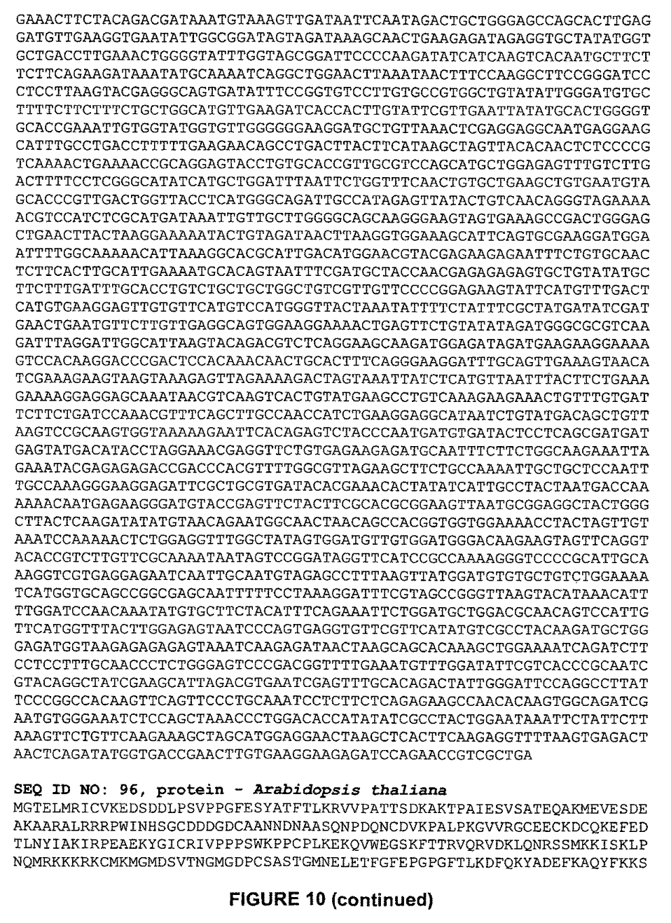

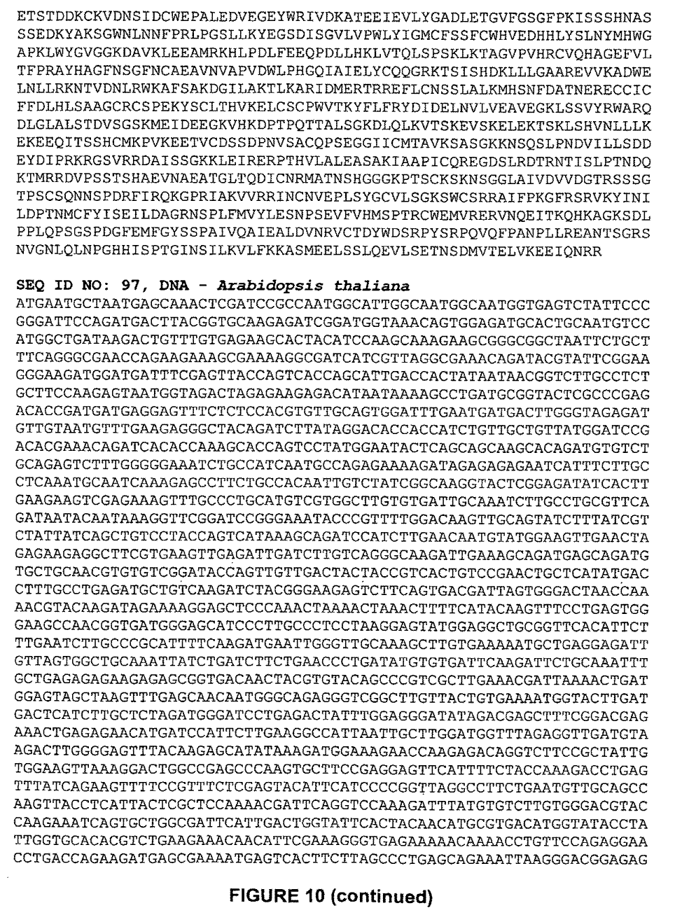

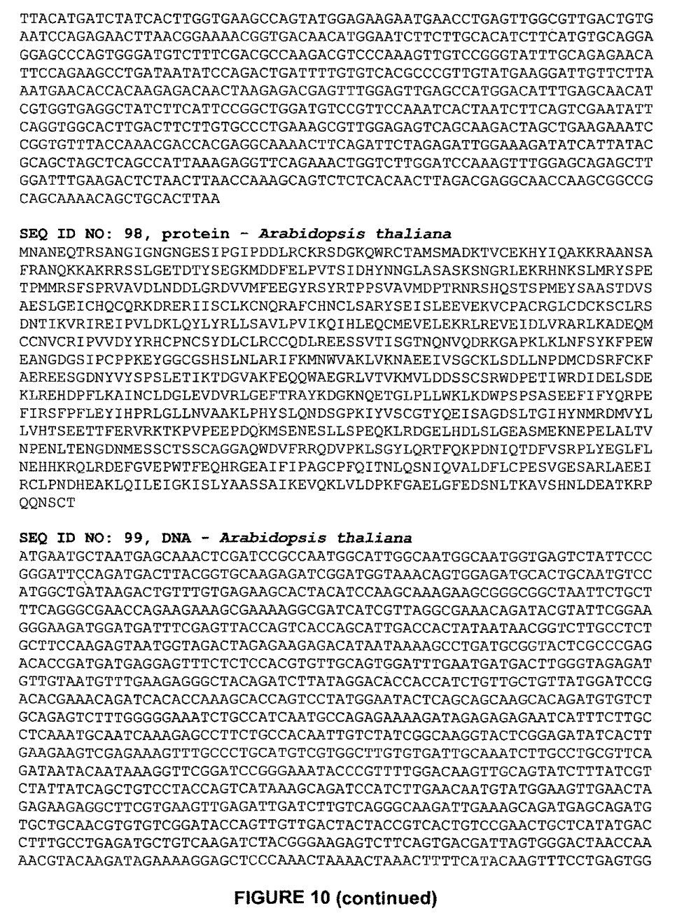

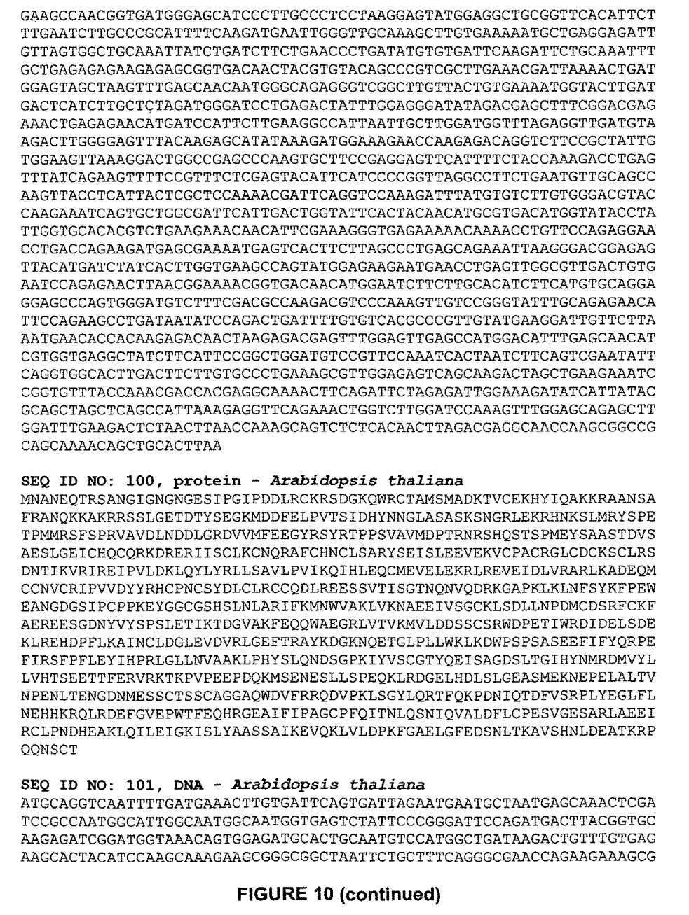

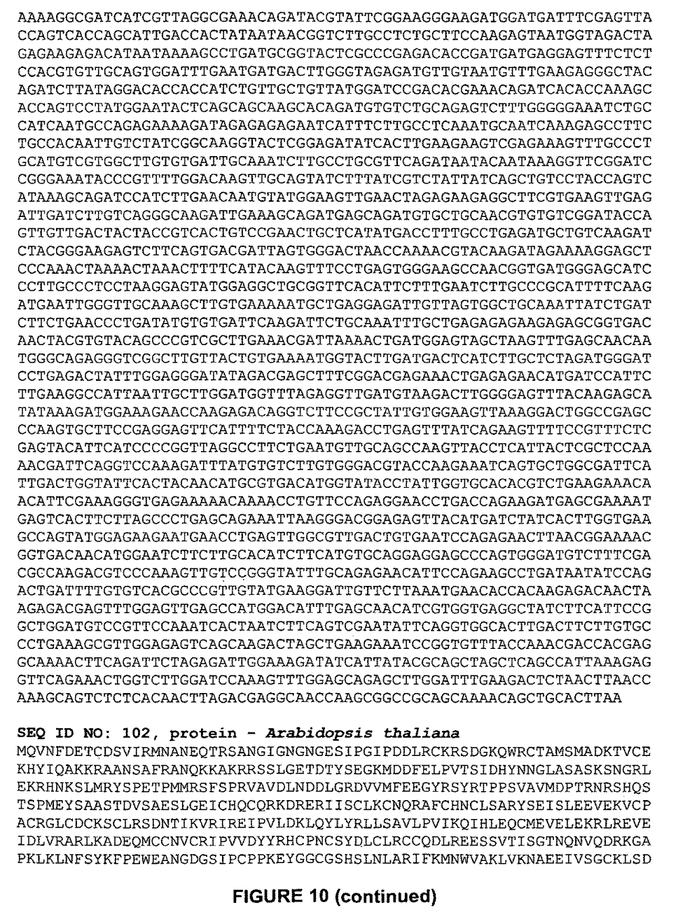

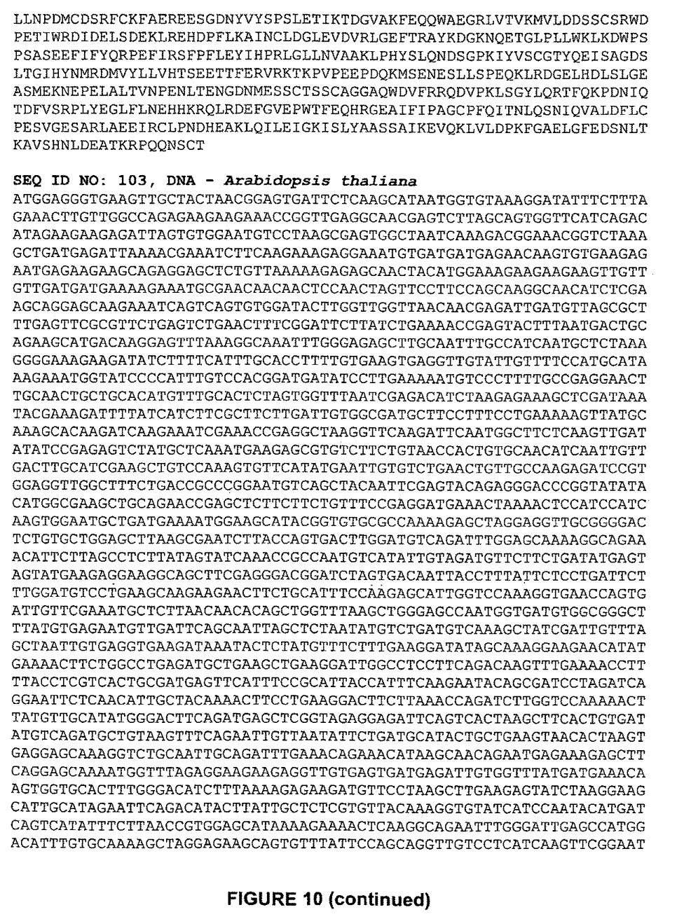

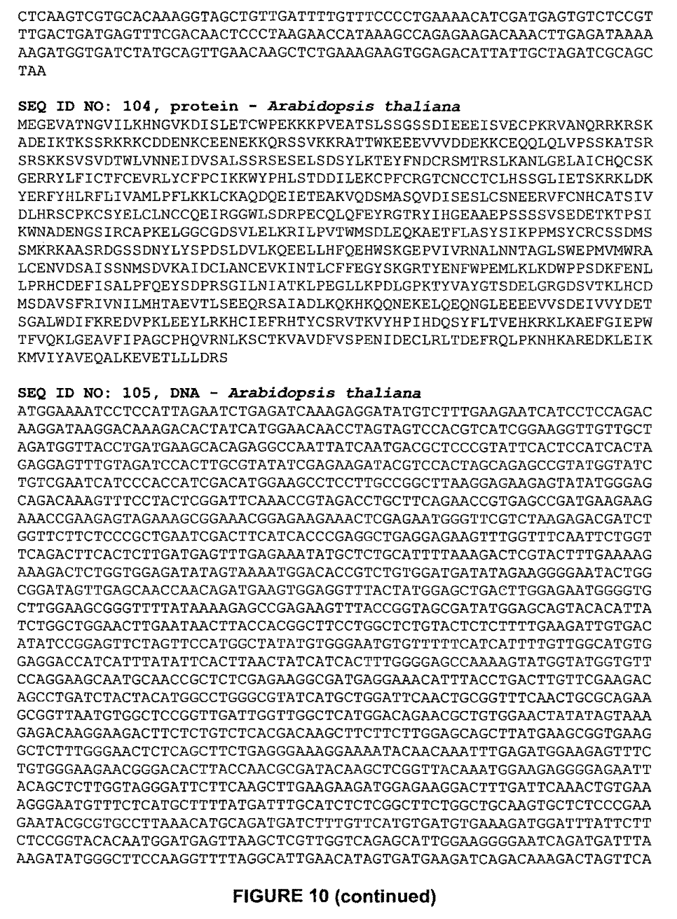

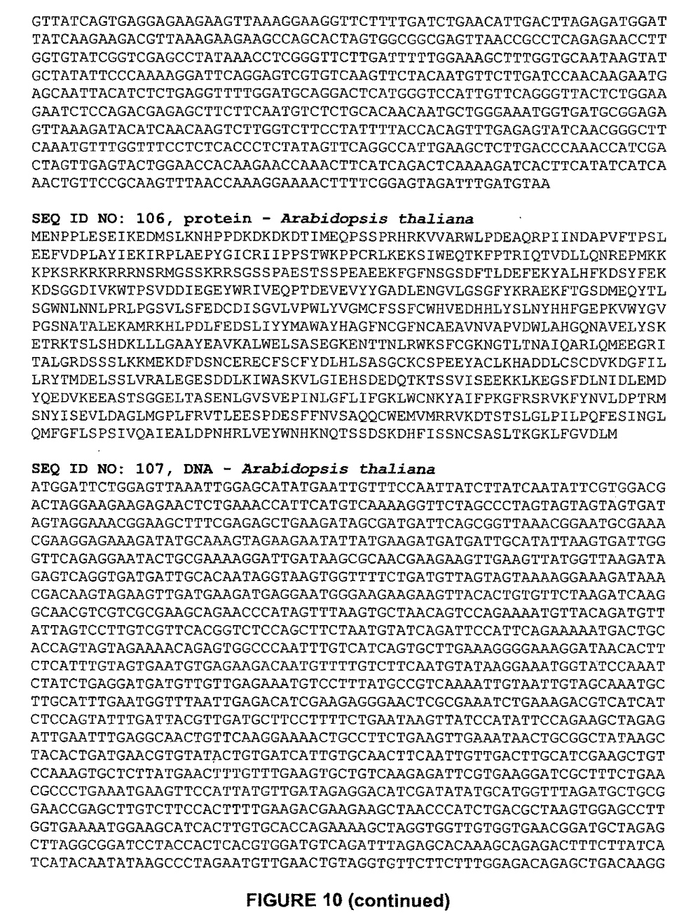

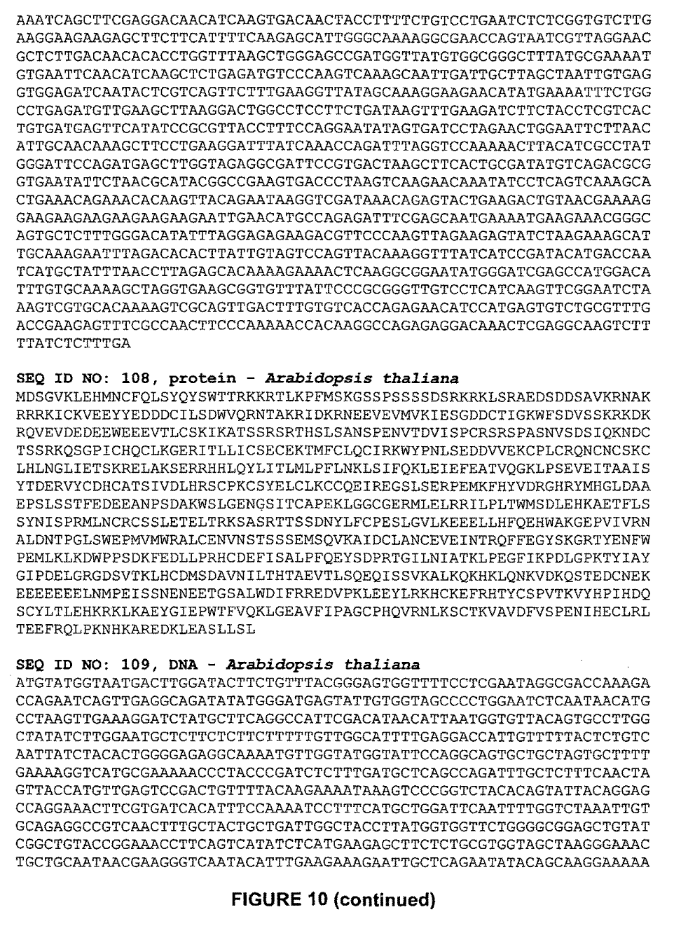

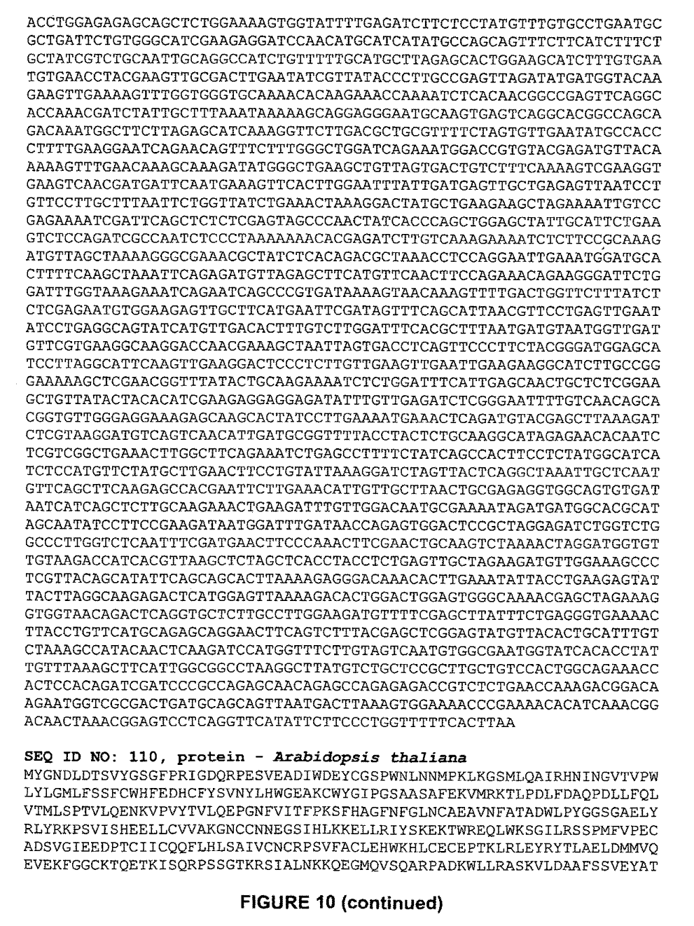

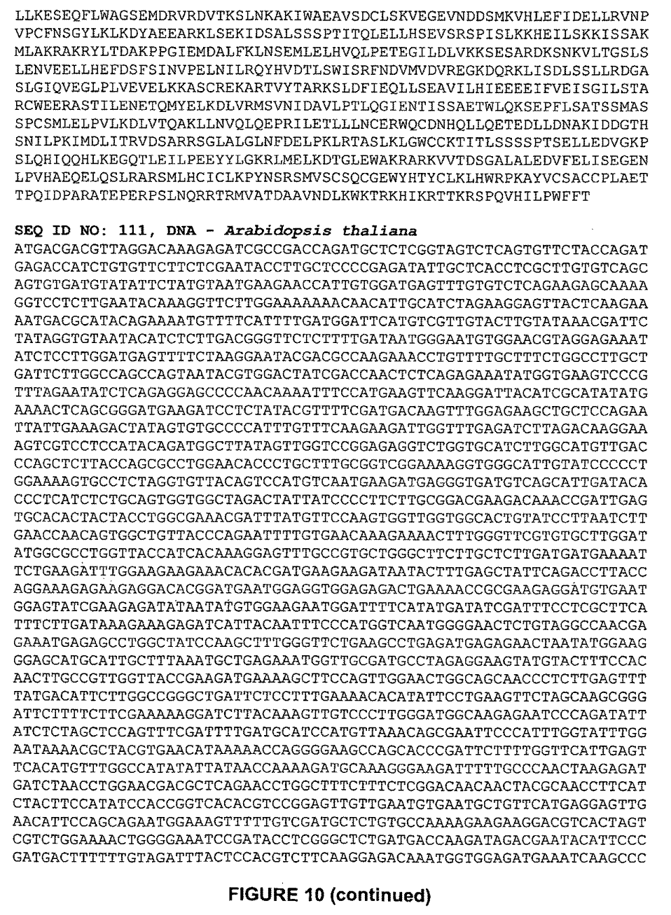

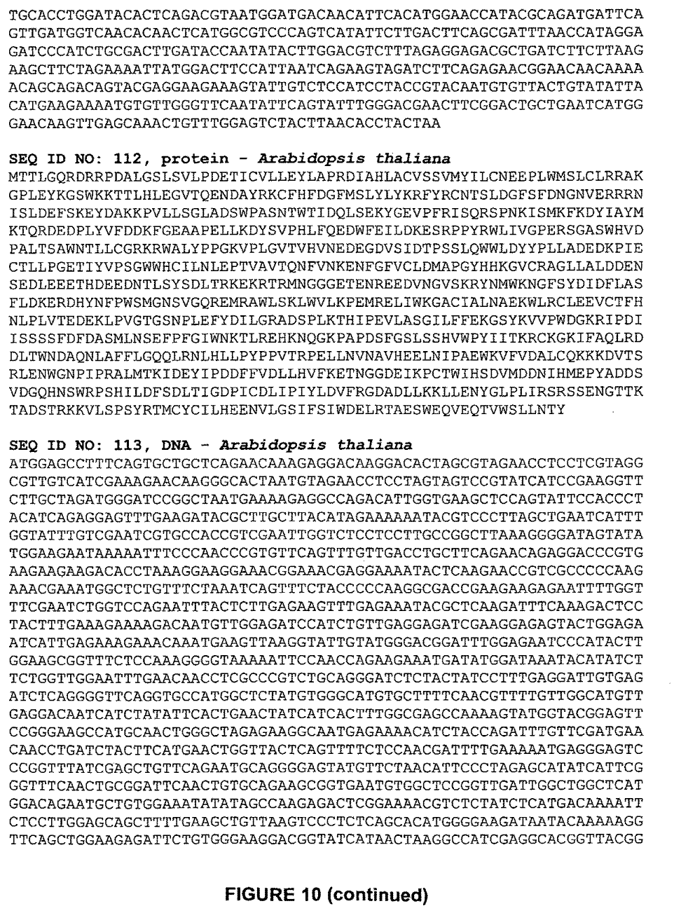

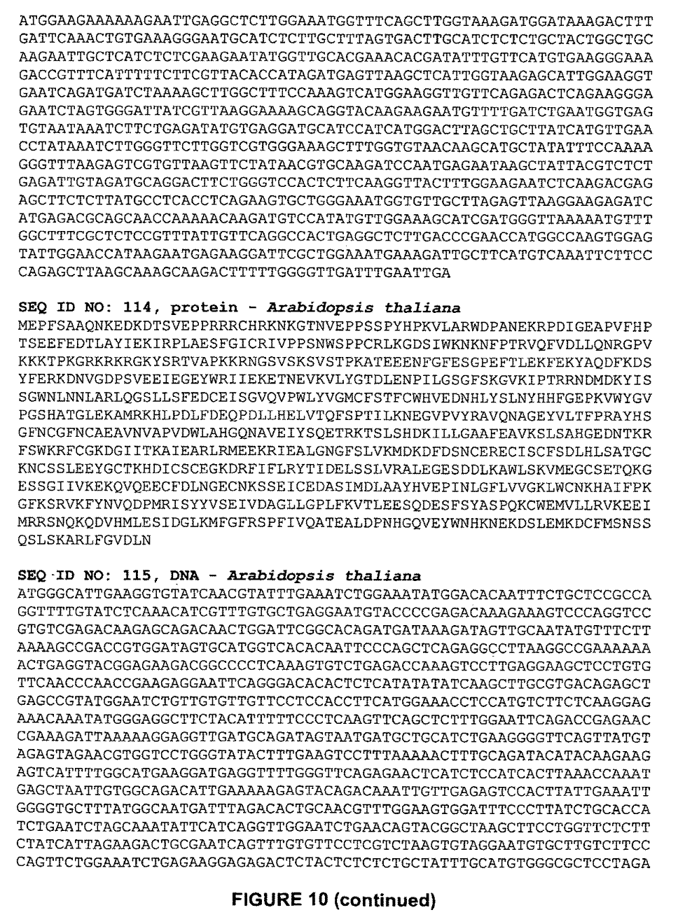

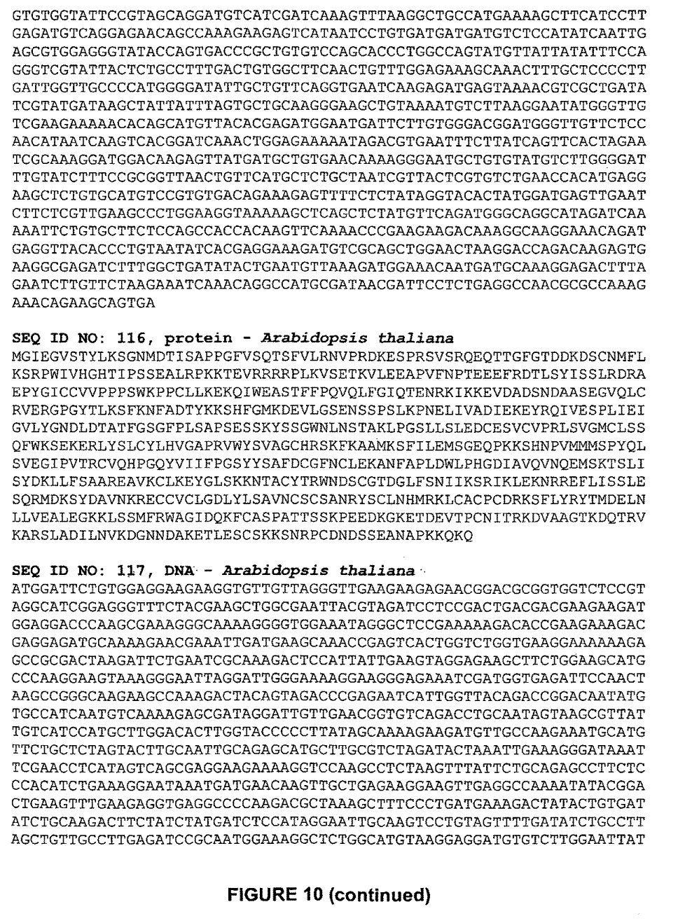

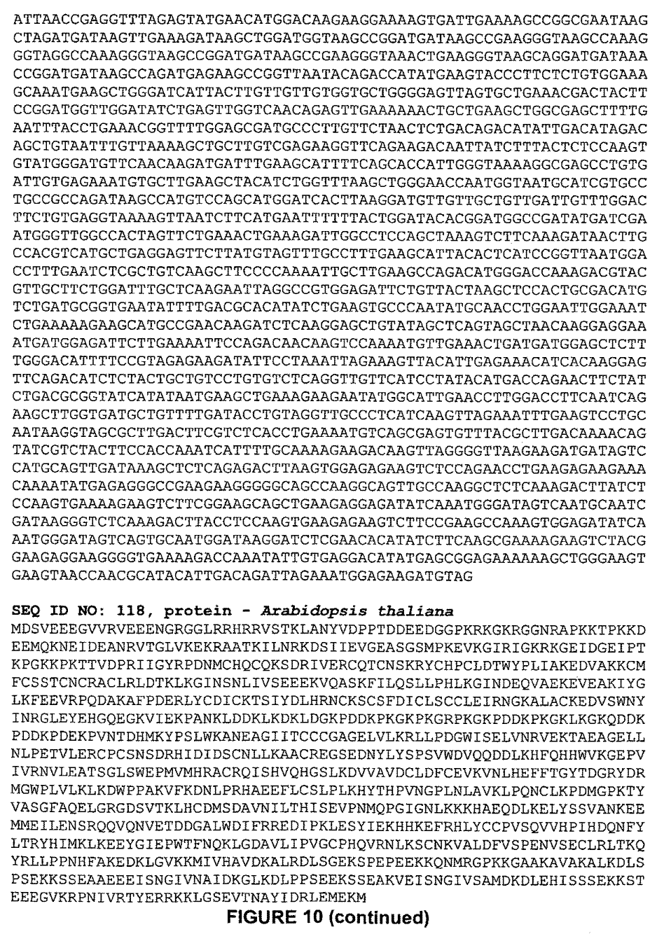

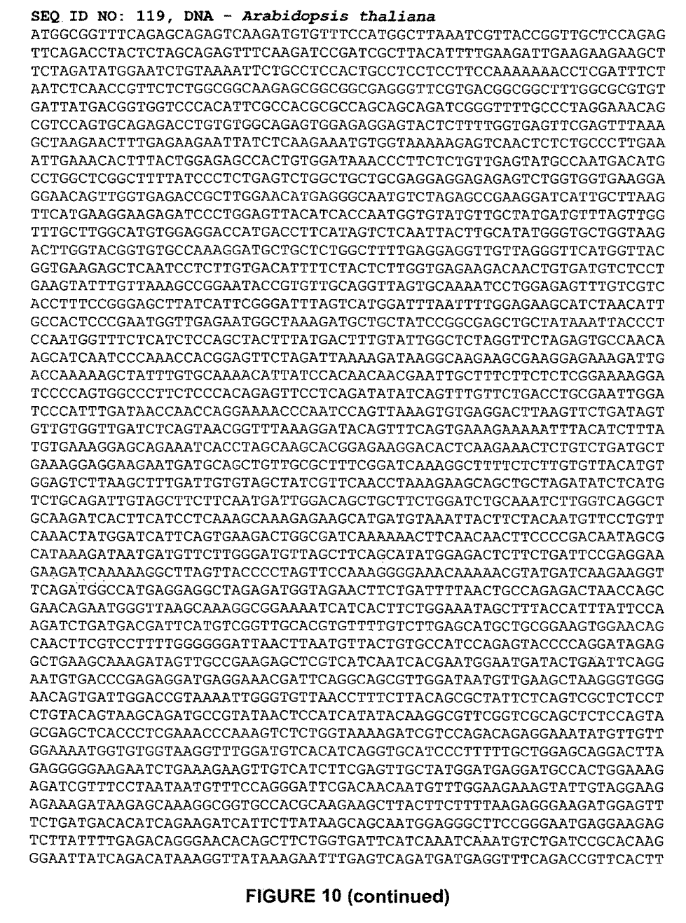

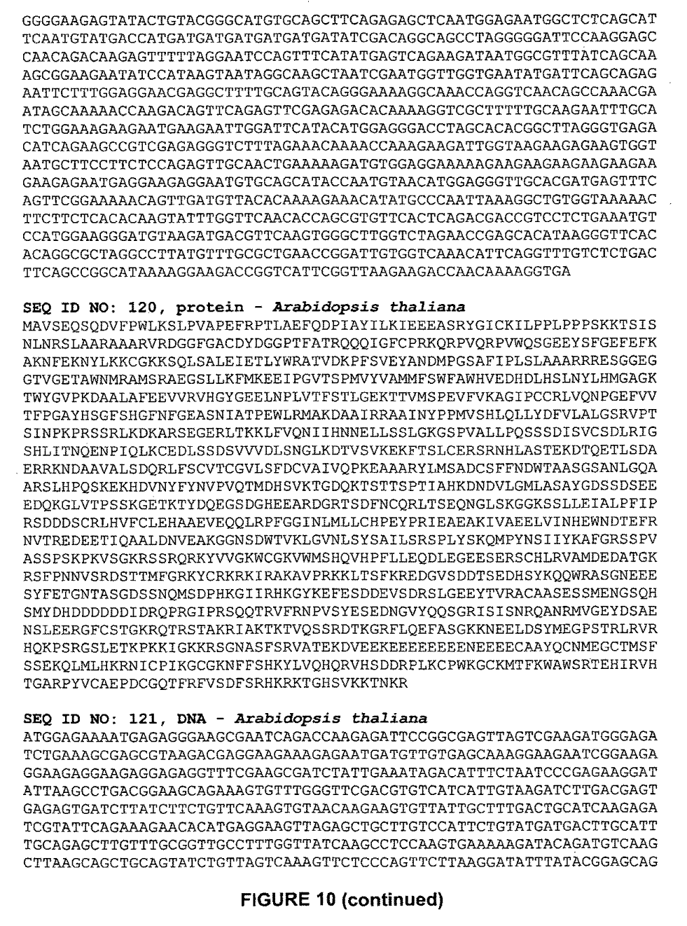

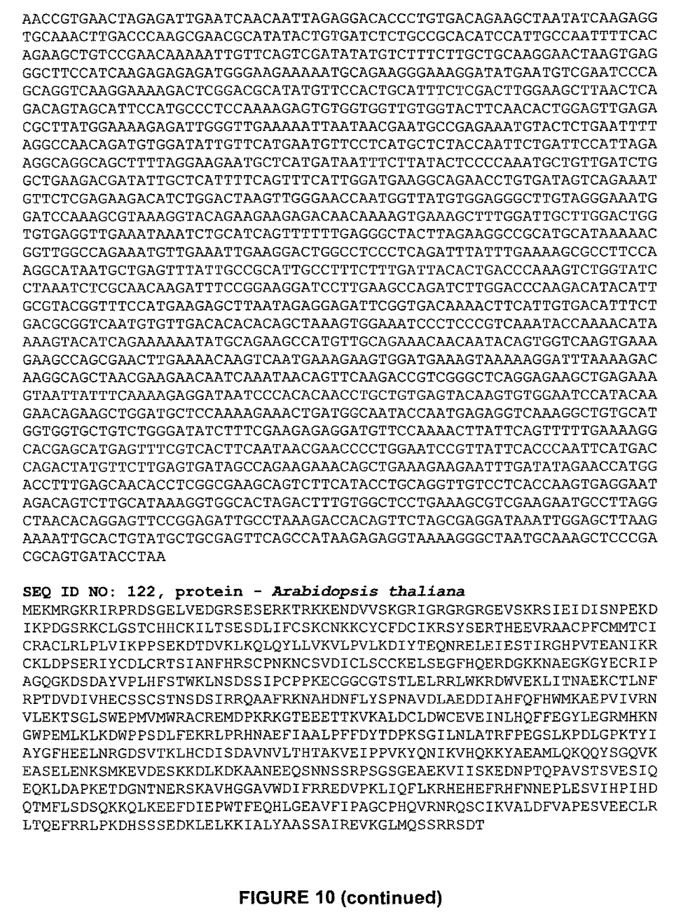

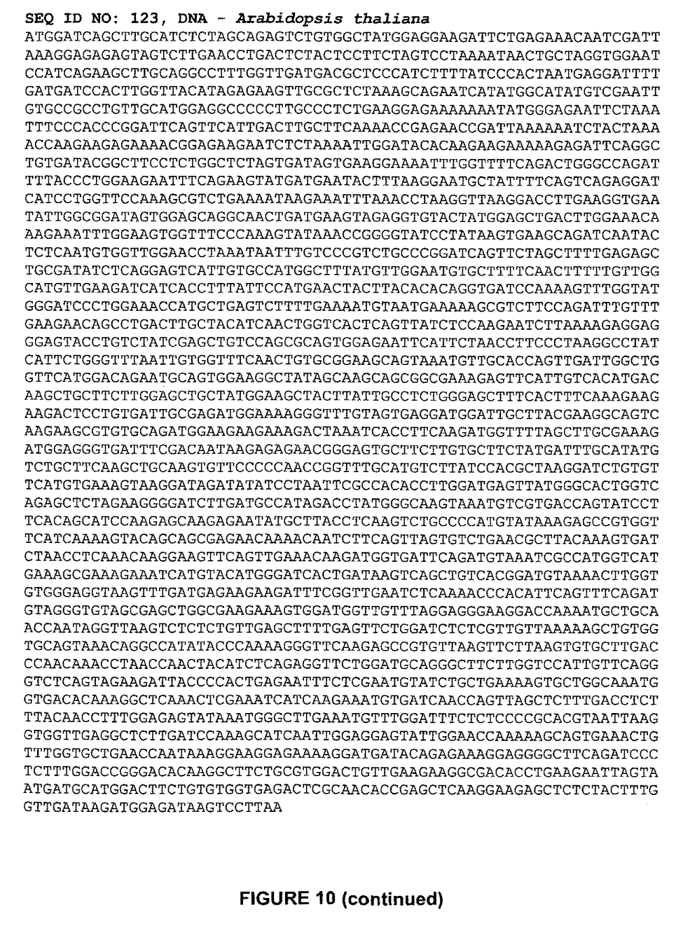

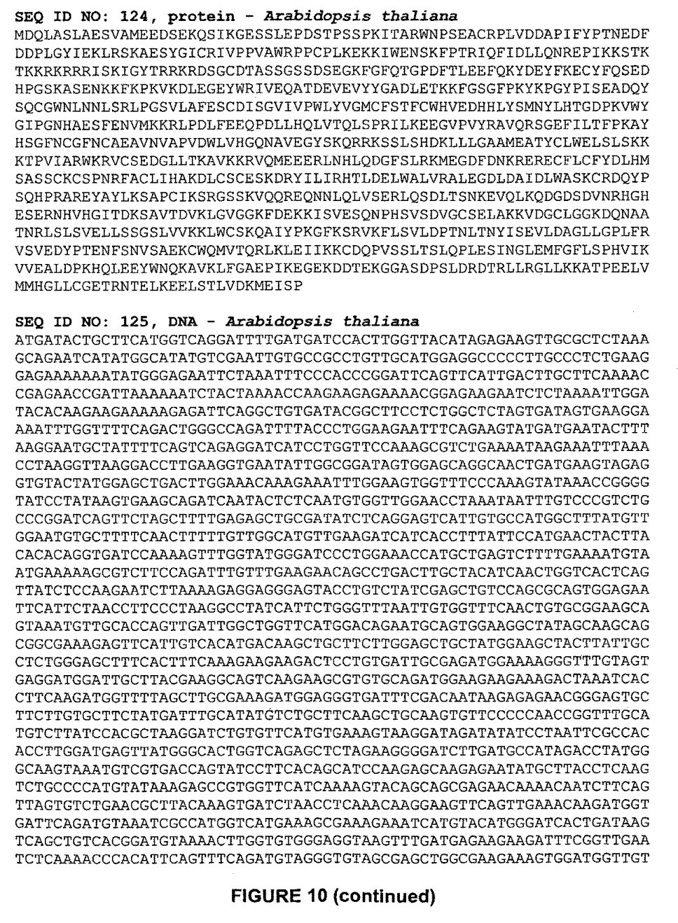

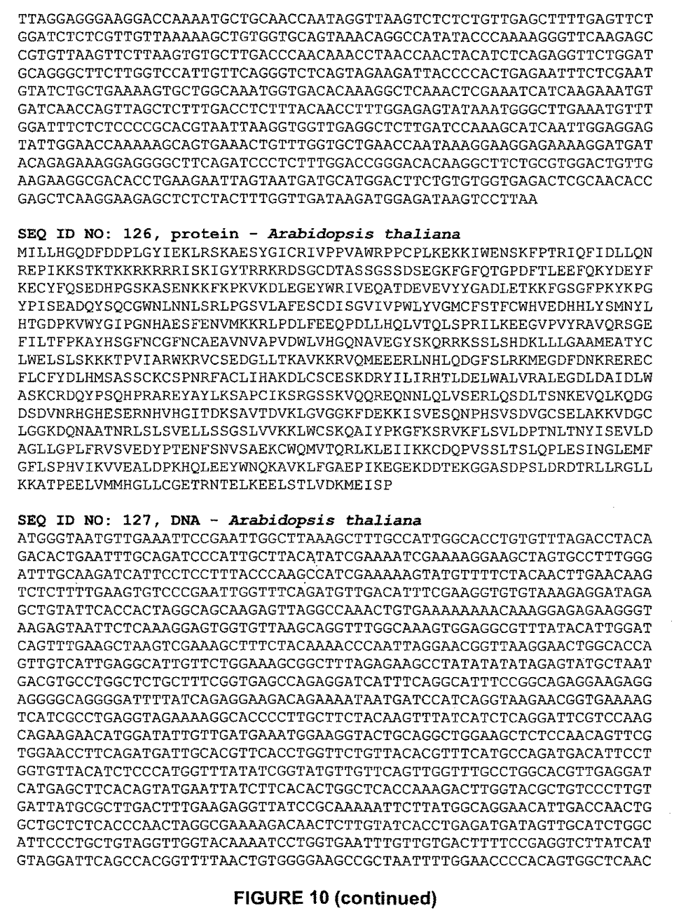

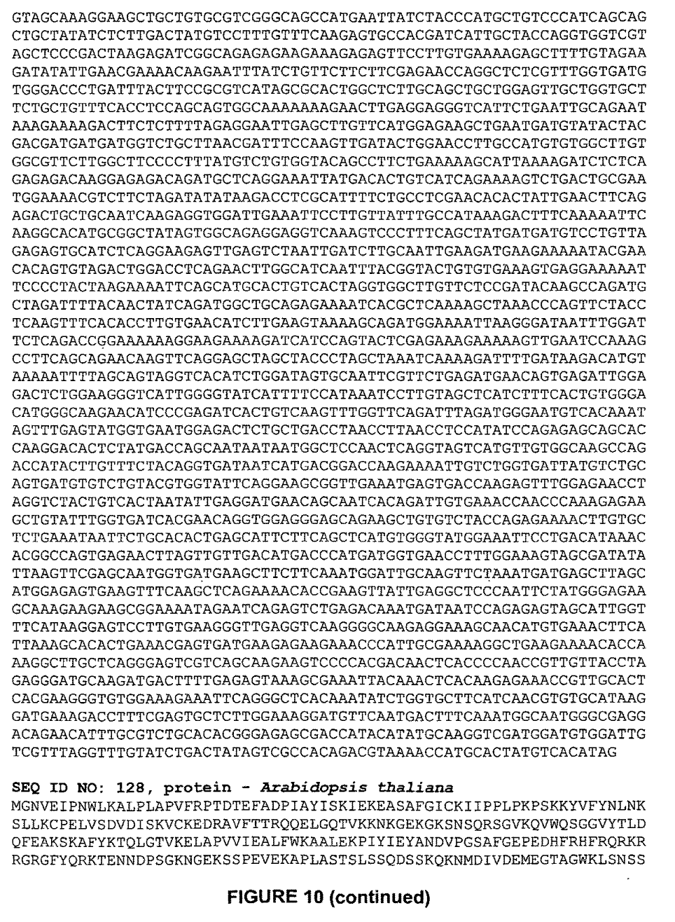

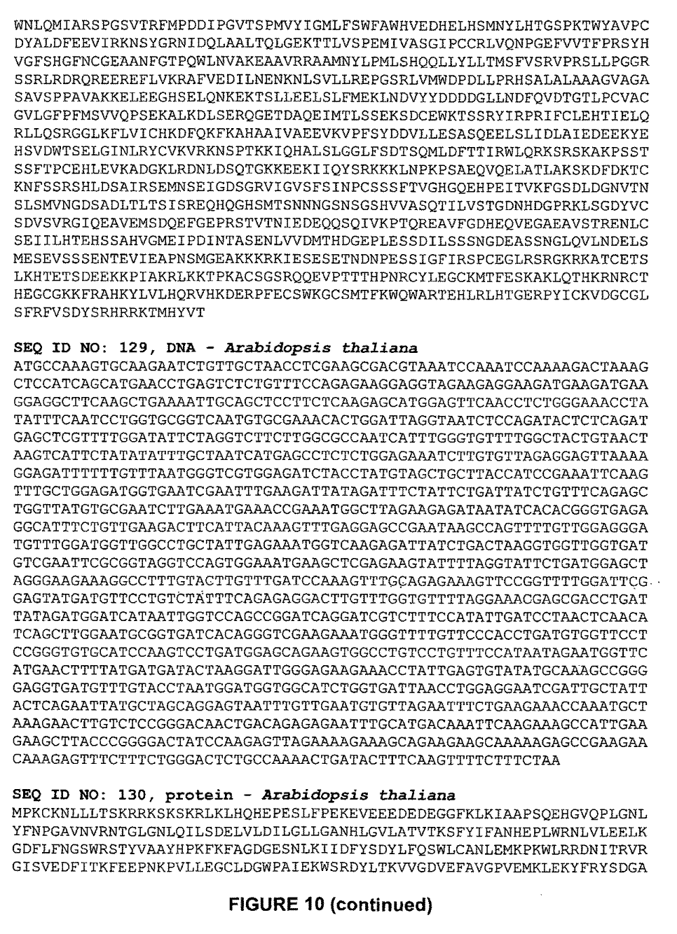

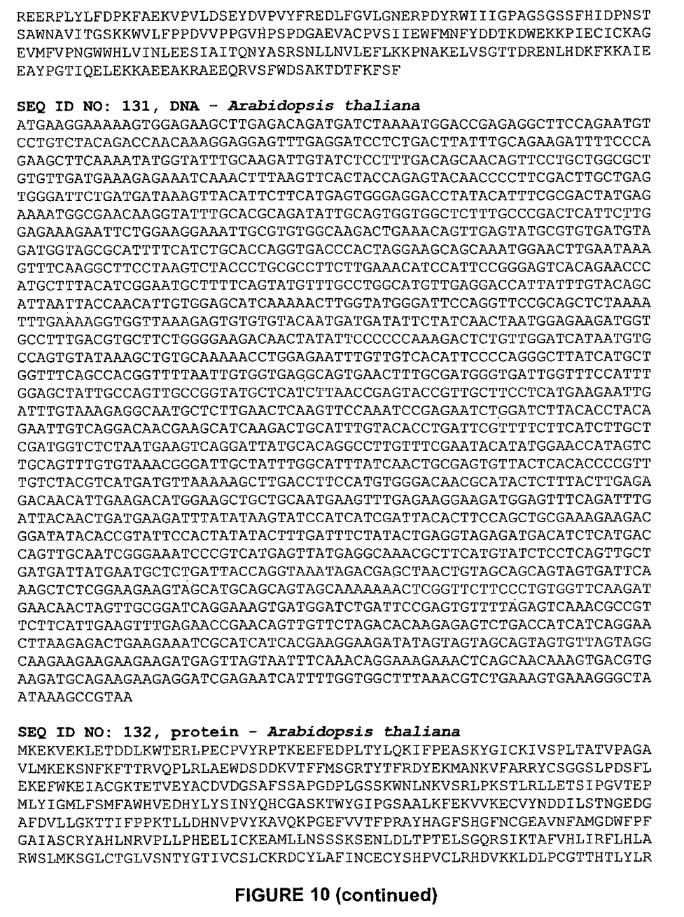

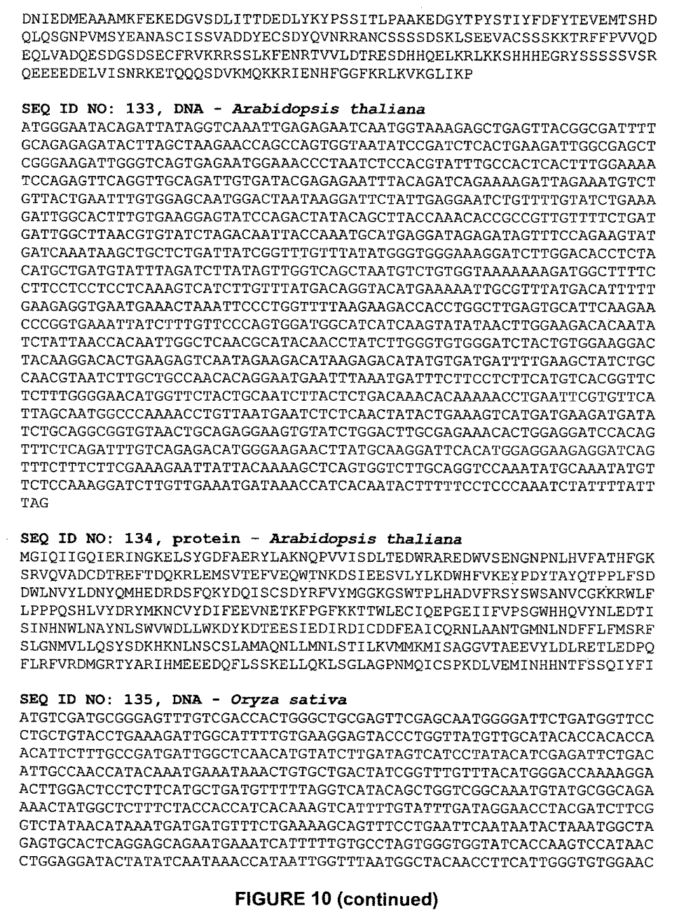

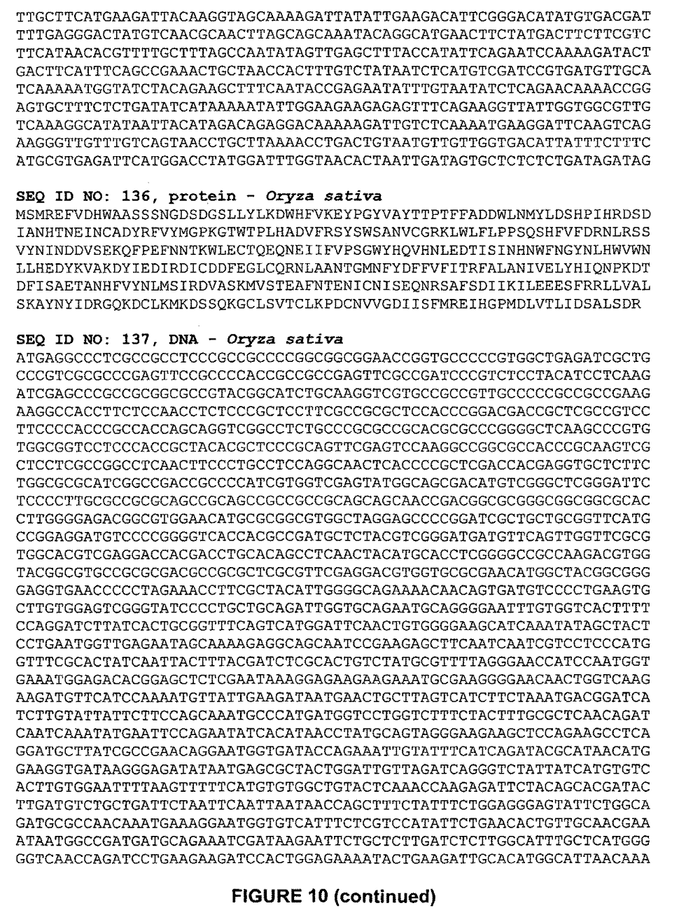

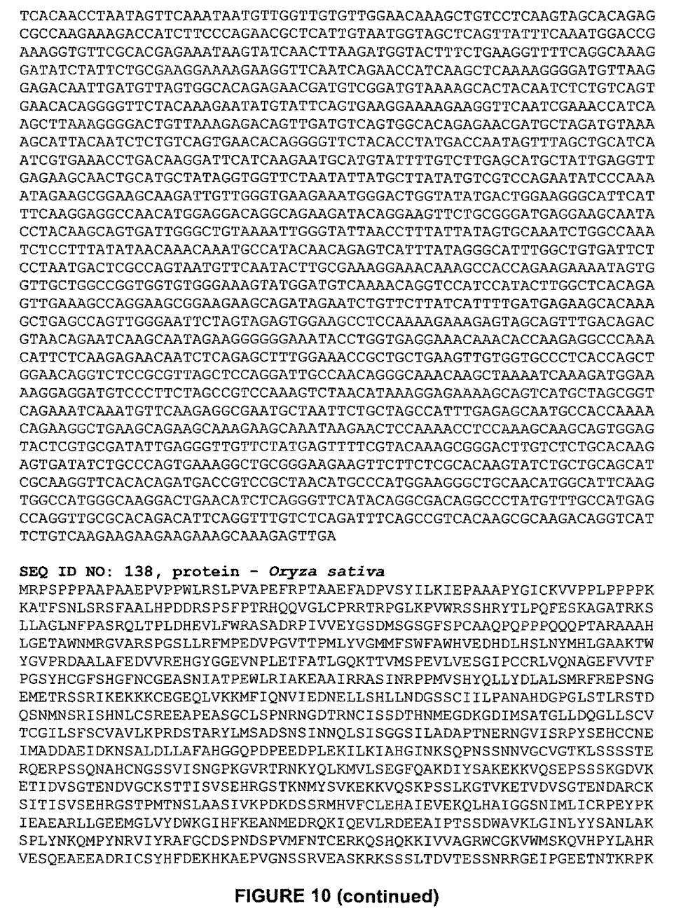

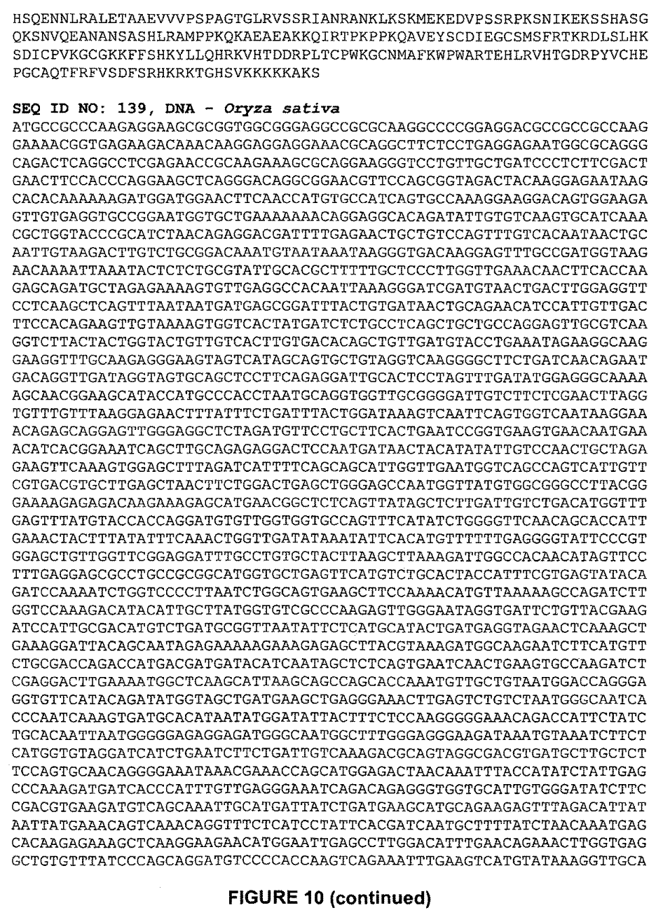

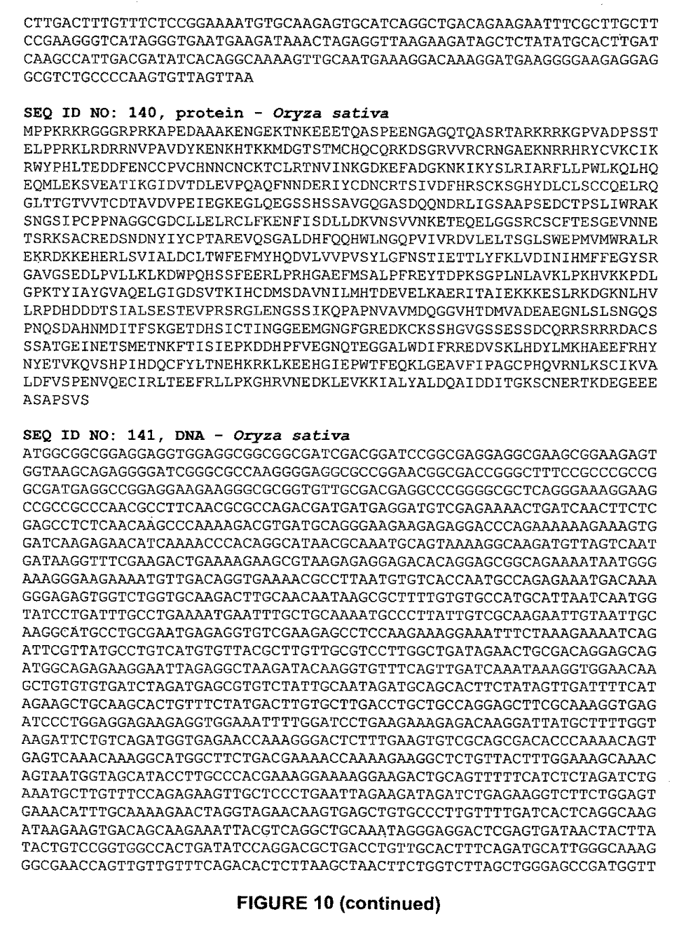

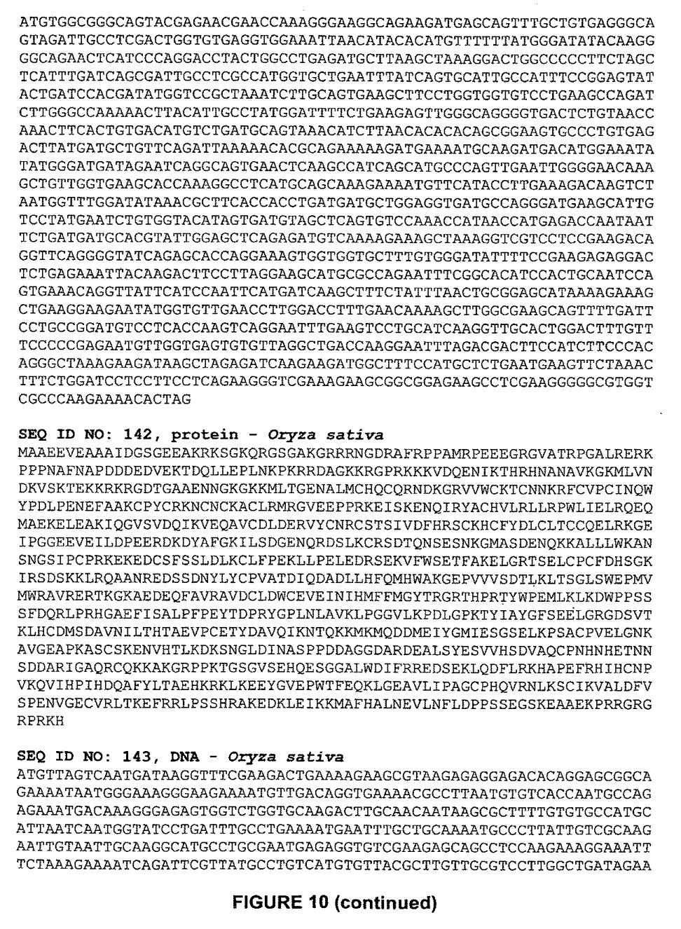

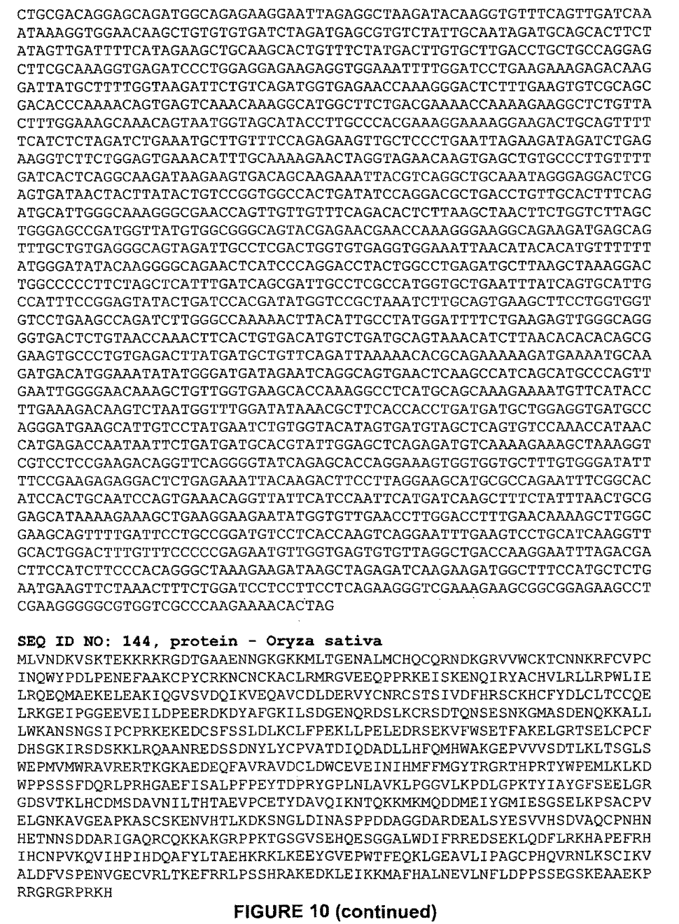

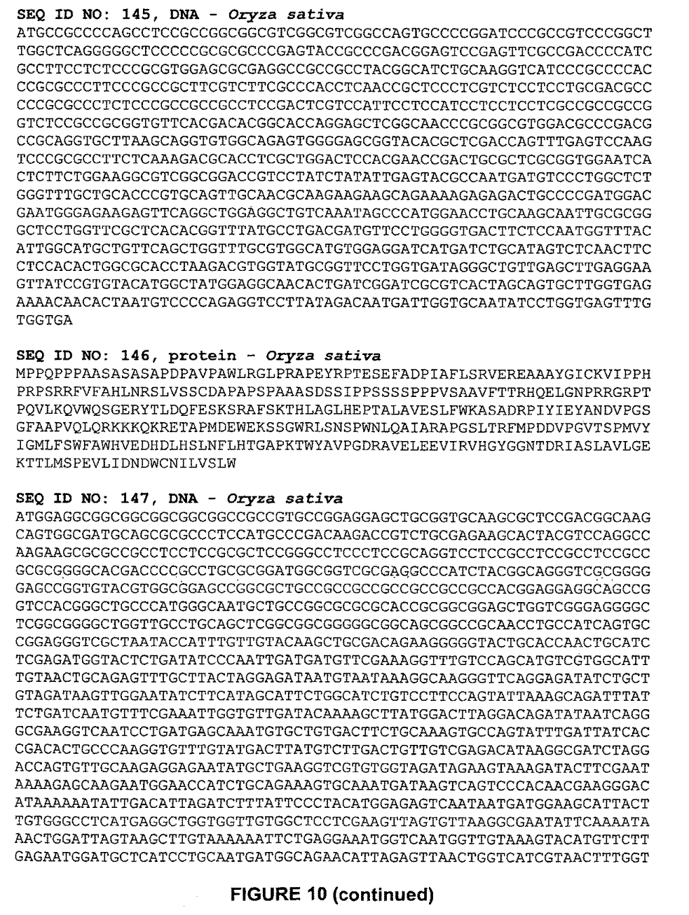

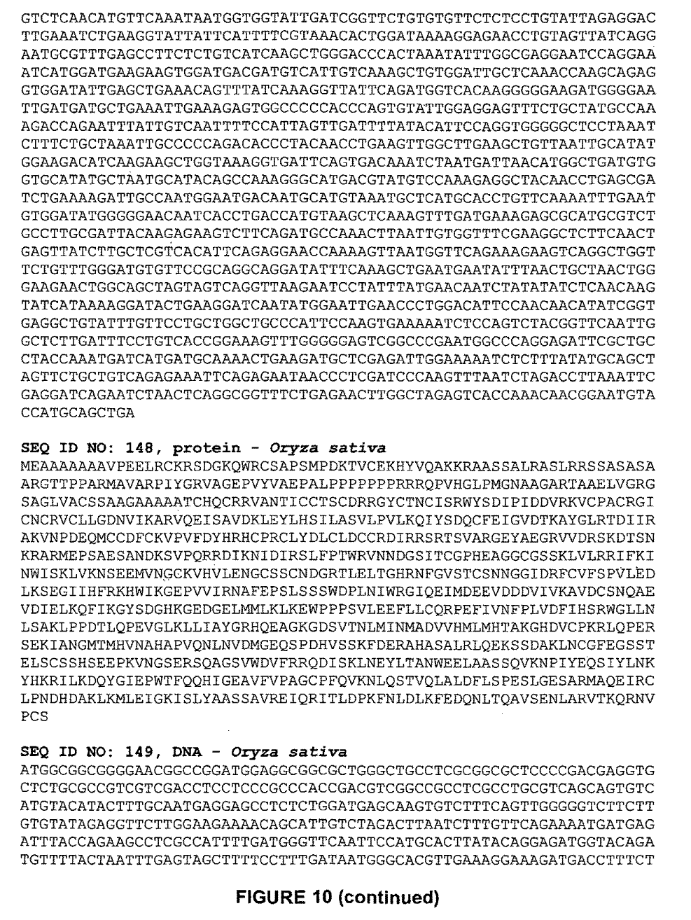

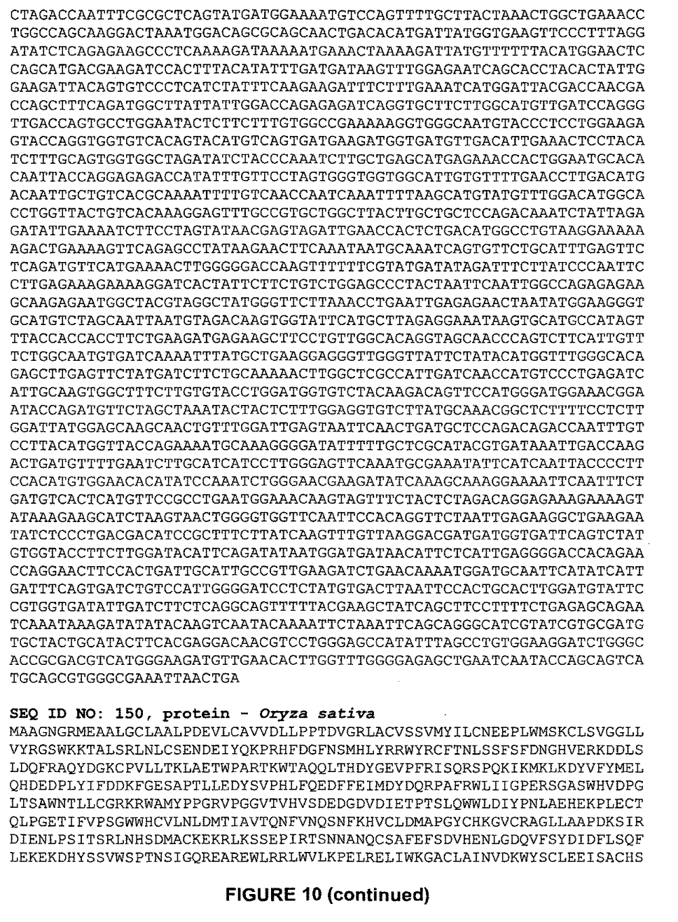

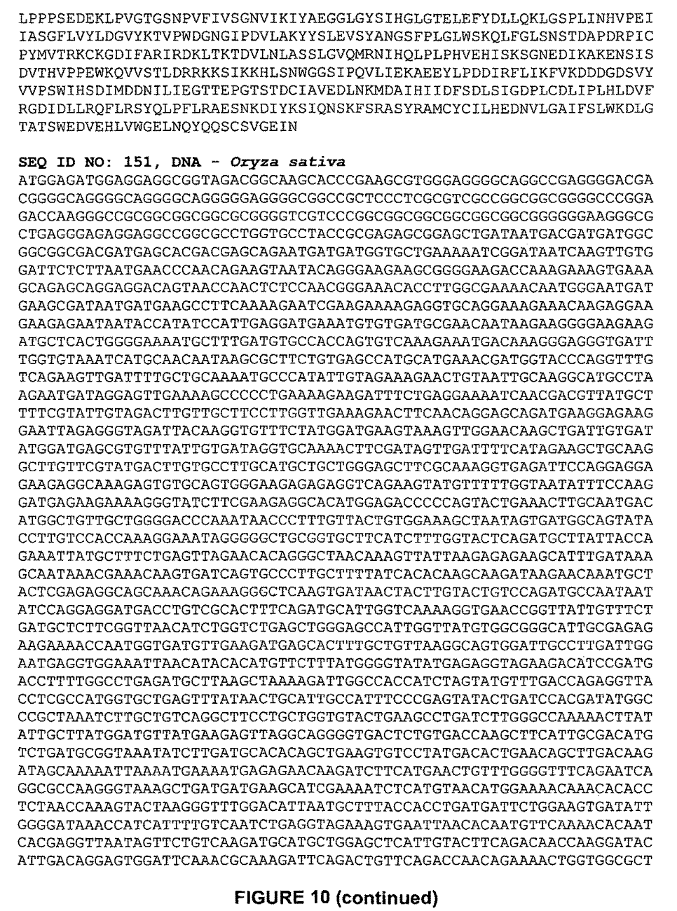

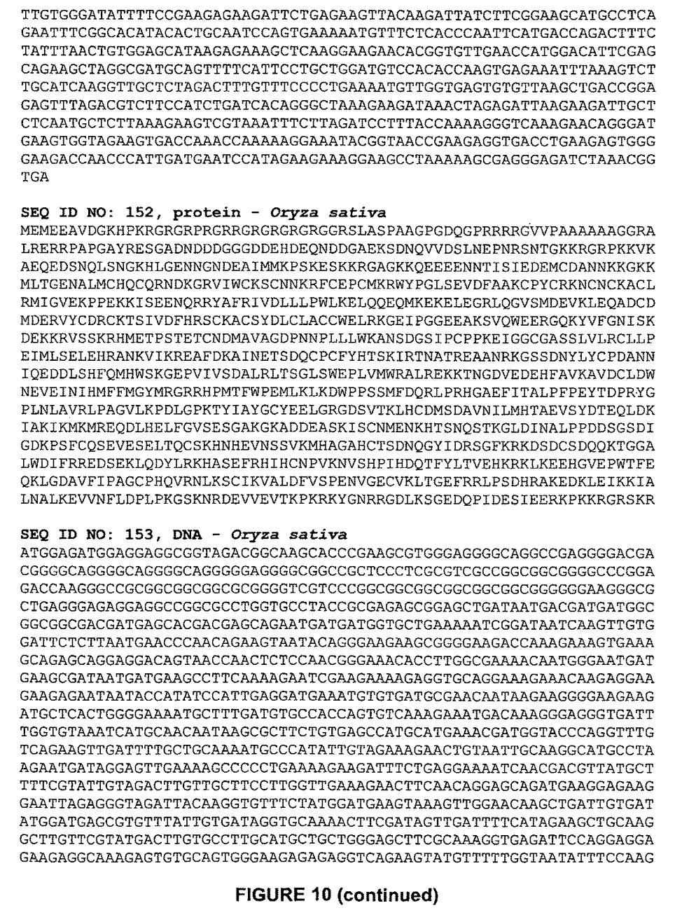

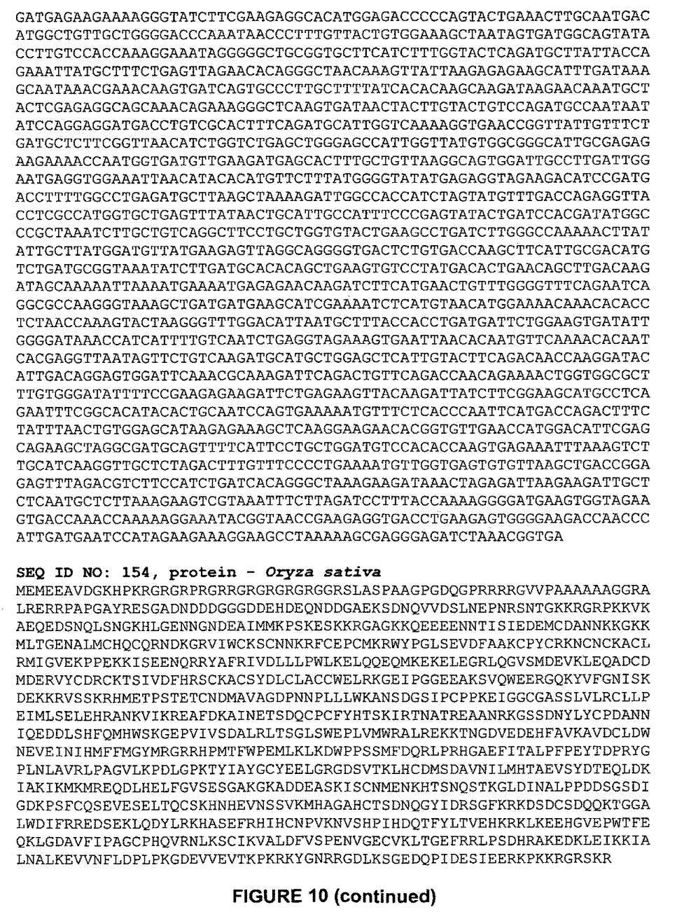

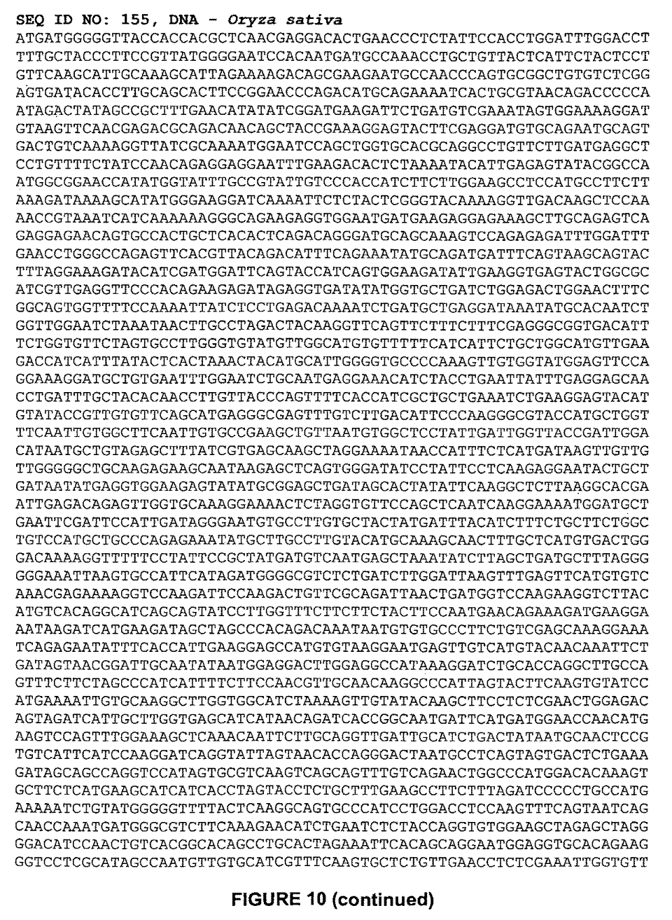

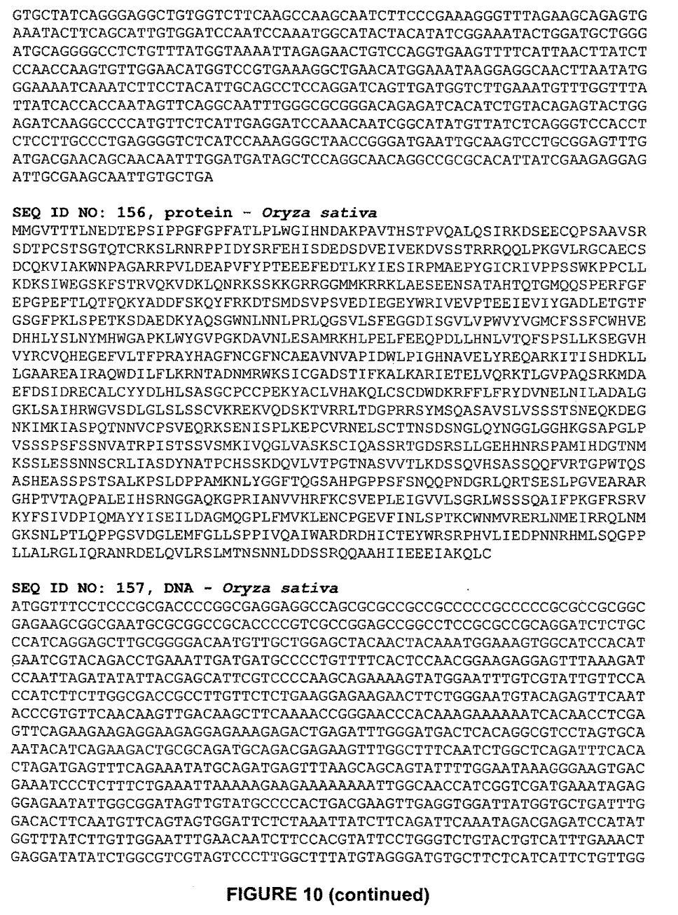

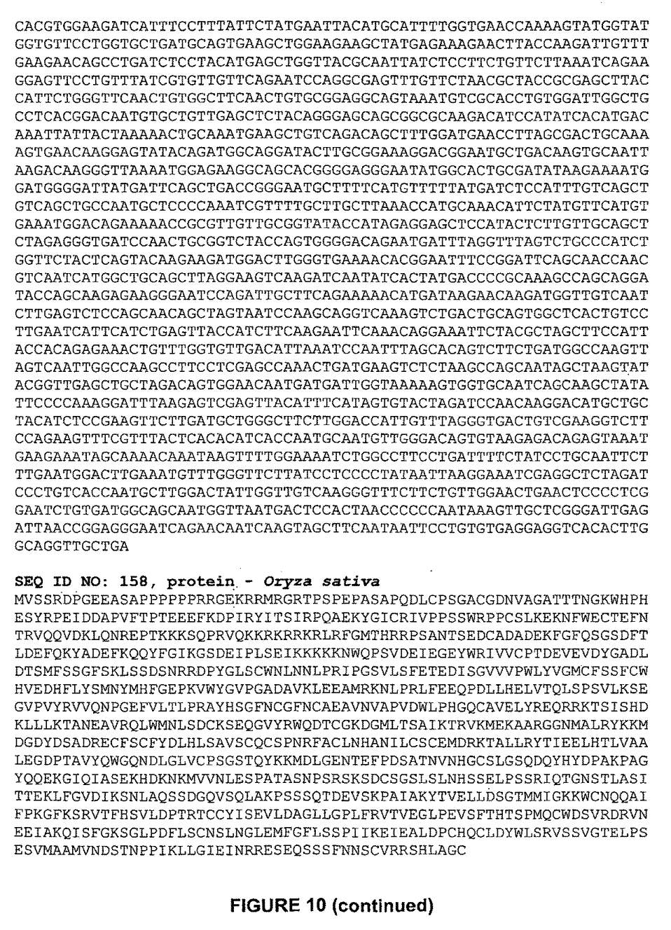

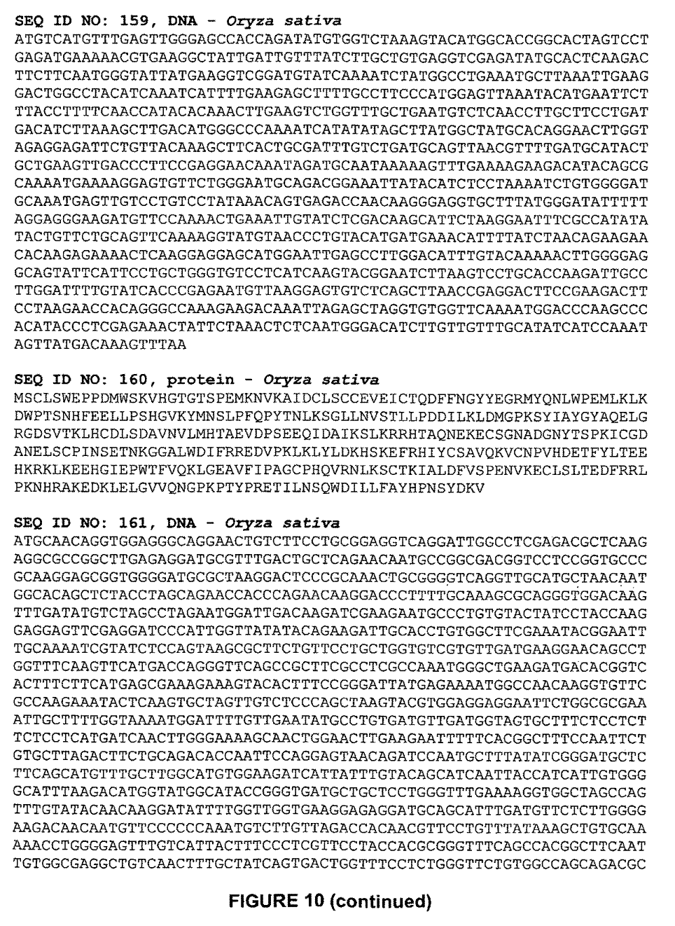

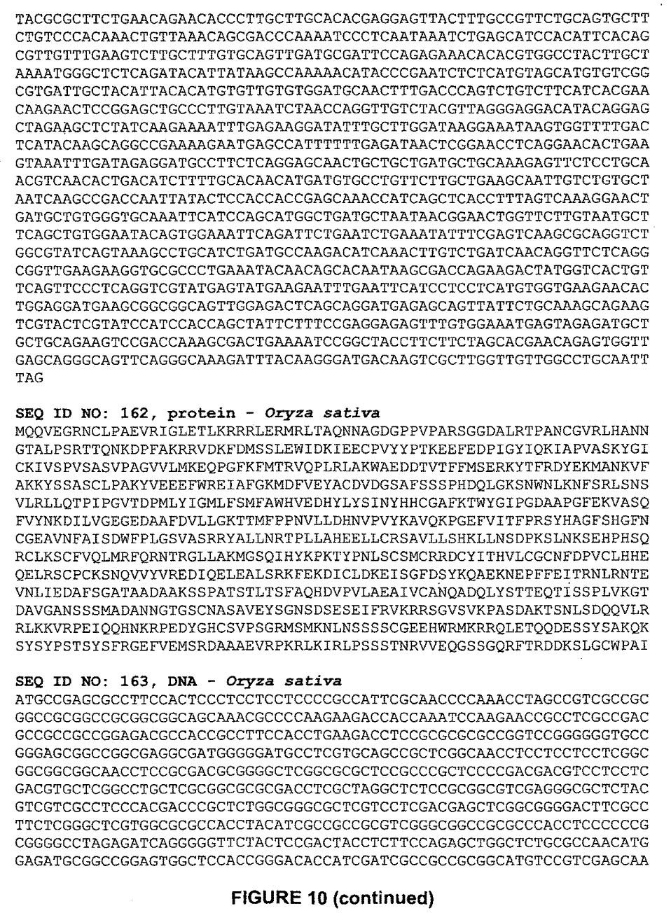

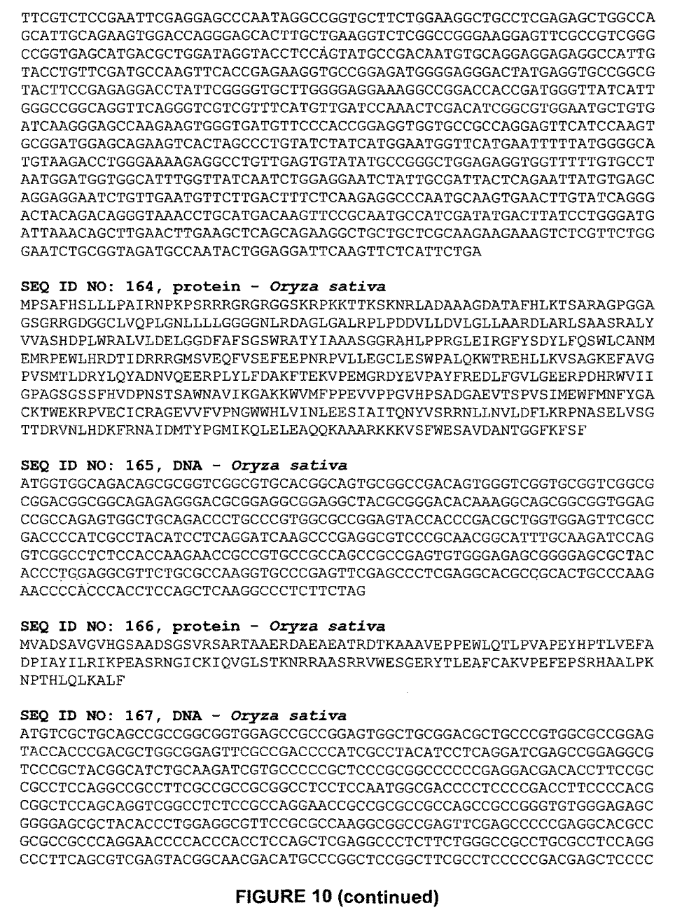

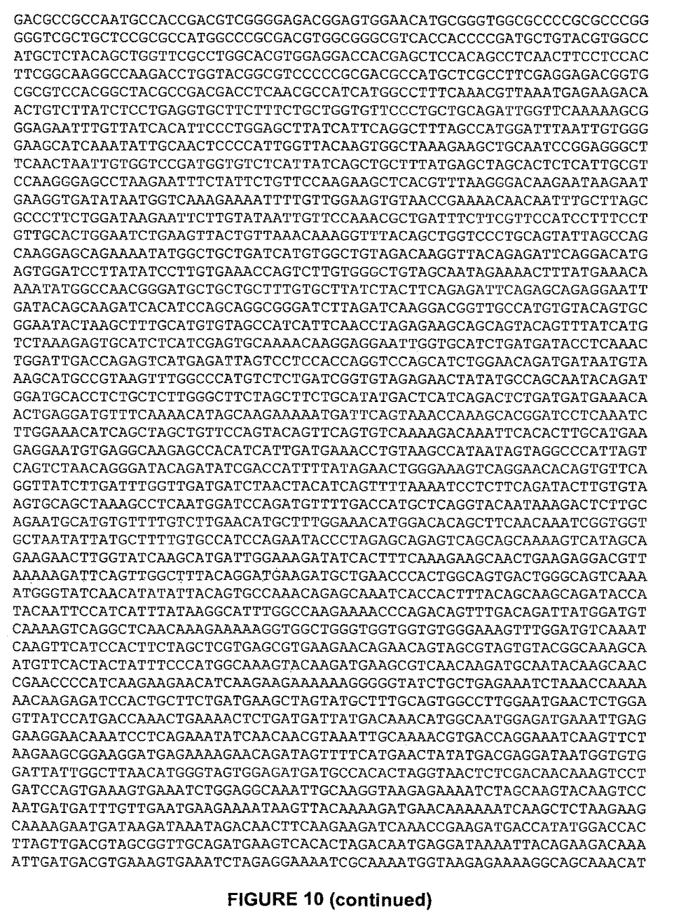

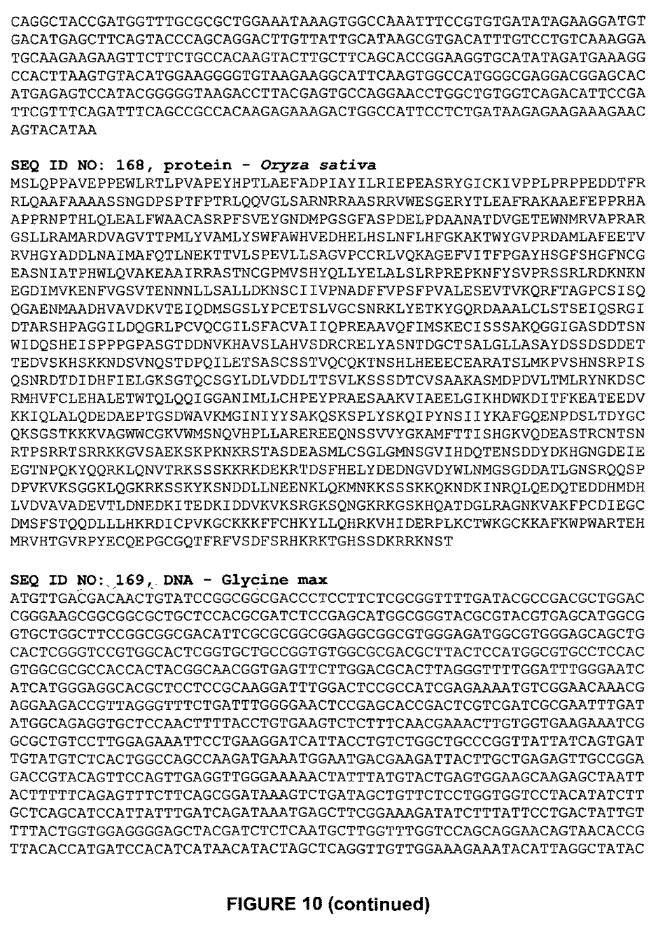

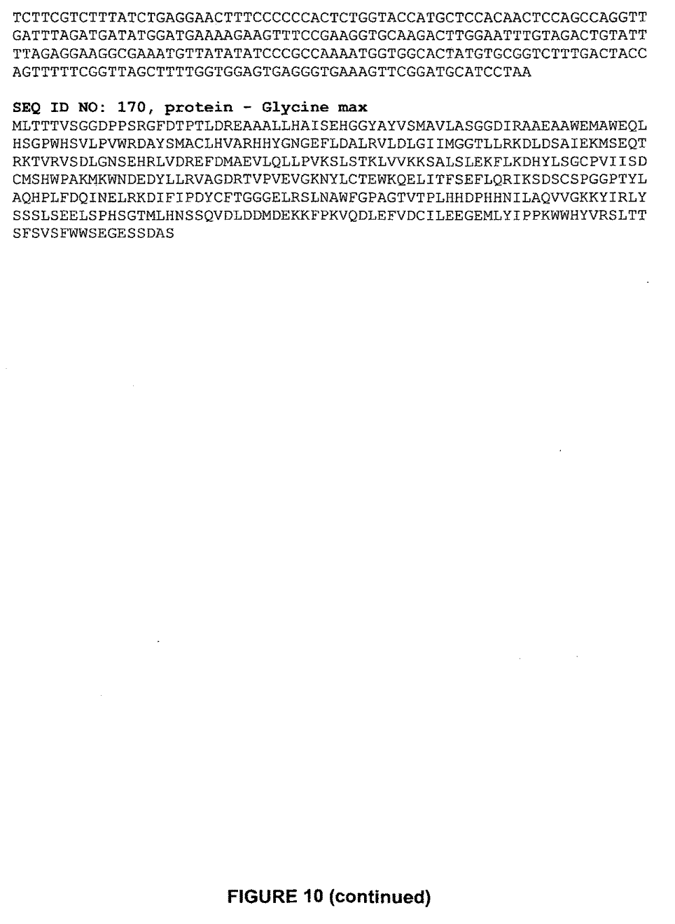

FIG. 10 details examples of JMJC sequences useful in performing the methods according to the present invention.

### III. Casein Kinase I

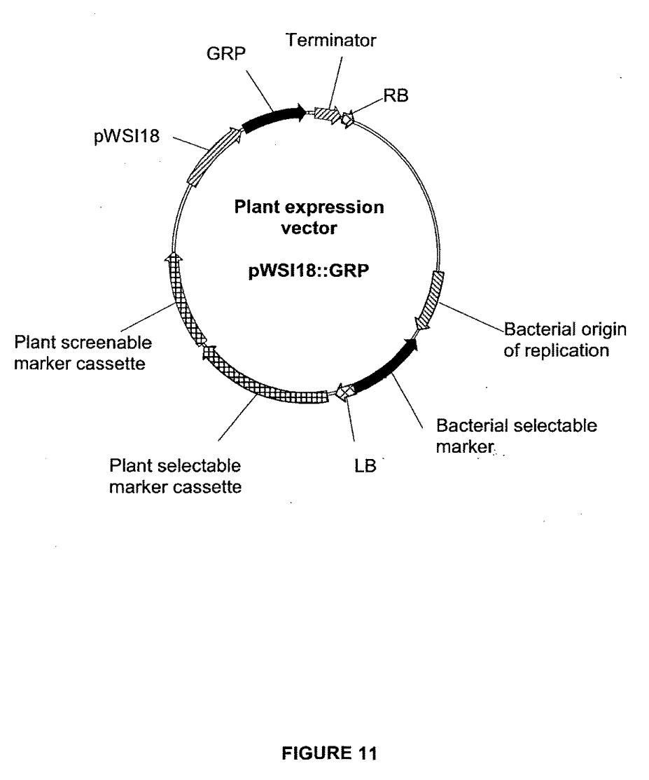

FIG. 11 represents the binary vector for increased expression in Oryza sativa of a GRP-encoding nucleic acid under the control of a rice WSI18 promoter (pWSI18::GRP)

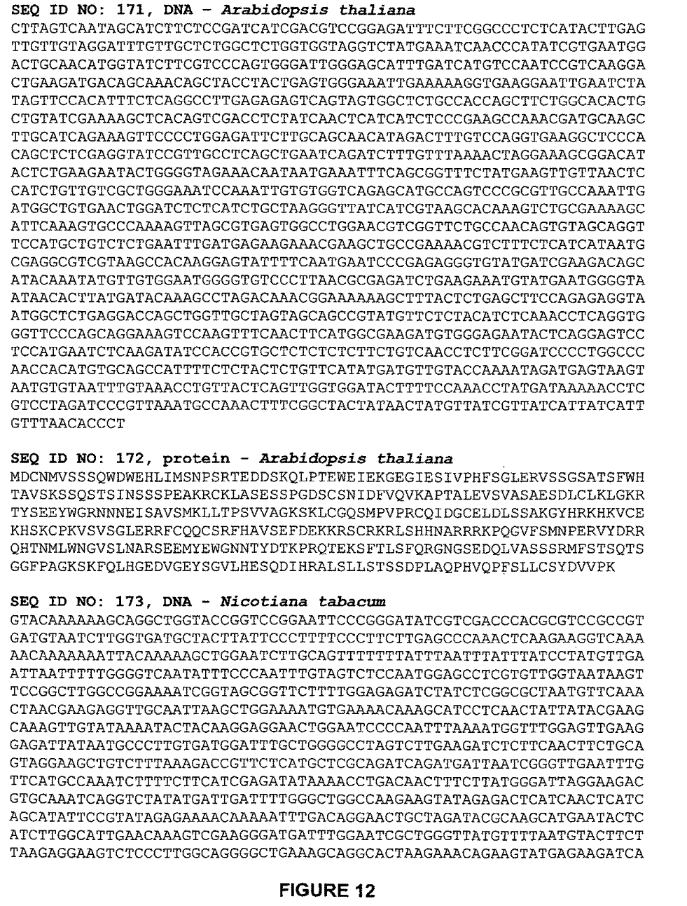

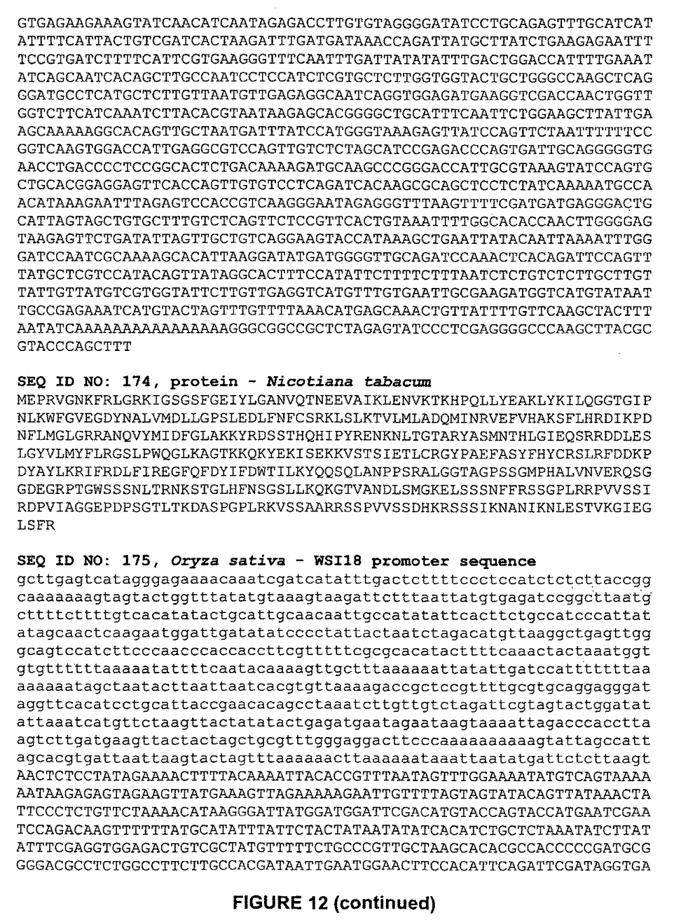

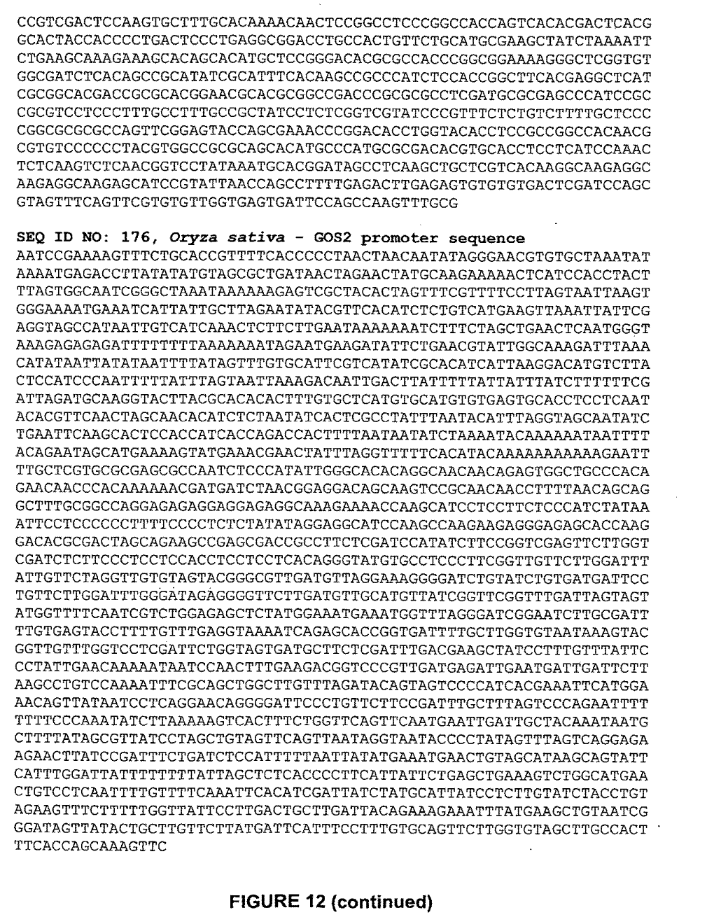

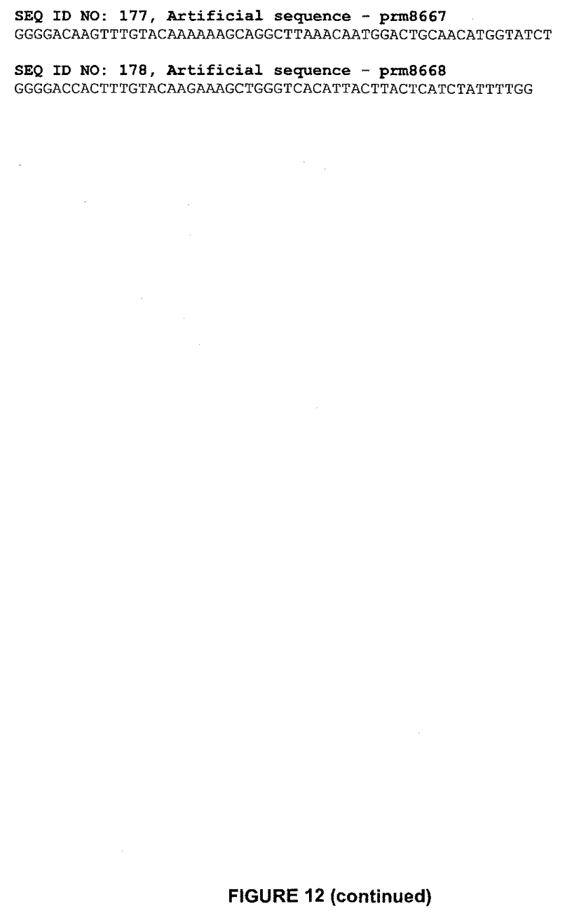

FIG. 12 details examples of GRP sequences useful in performing the methods according to the present invention.

### IV. Plant Homeodomain Finger-Homeodomain (PHDf-HD) Polypeptide

FIG. 13 represents a neighbour-joining tree constructed after an alignment of all the transcription factors belonging to the HD family (downloaded from the riceTFDB database hosted at the server of the University of Potsdam) and all of the polypeptide sequences of Table D1 (when full length), performed using the Clustal algorithm (1.83) of progressive alignment, using default values. The group of interest, comprising the two rice paralogs (SEQ ID NO: 180 or Os02g05450.1, and SEQ ID NO: 202 or Os06g12400.1) has been circled. Any polypeptide falling within this HD group (after a new multiple alignment step as described hereinabove) is considered to be useful in performing the methods of the invention as described herein.

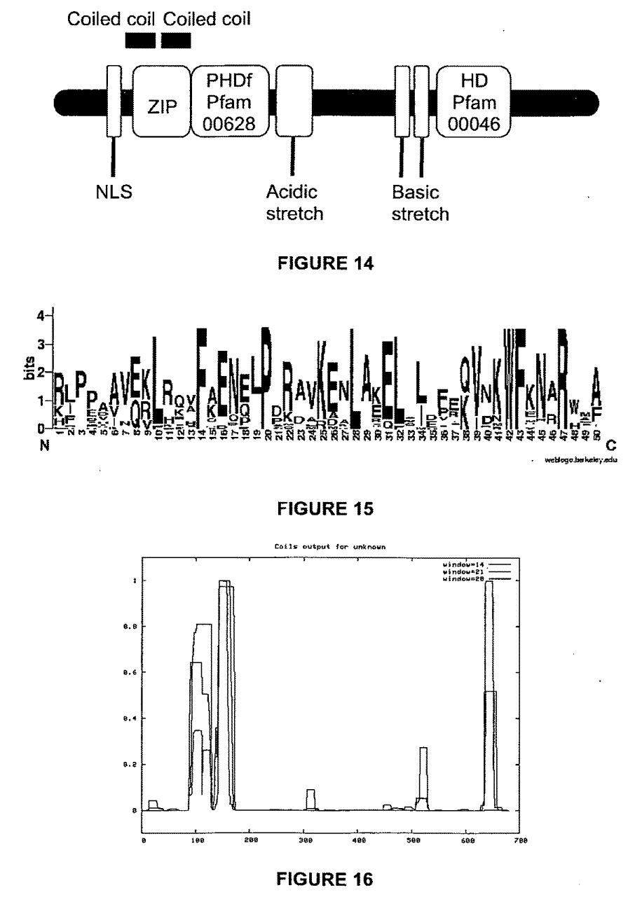

FIG. 14 represents a cartoon of a PHDf-HD polypeptide as represented by SEQ ID NO: 180, which comprises one or more of the following features: a predicted nuclear localisation signal (NLS), a leucine zipper (ZIP), a PHD finger (PHDf, PfamOD628), an acidic stretch, two basic stretches, a homeodomain (HD, Pfam00046).

FIG. 15 shows the sequence logo of the homeodomain (HID) of the PHDf-HD polypeptides of Table D1, where the overall height of the stack indicates the sequence conservation at that position, while the height of symbols within the stack indicates the relative frequency of each amino or nucleic acid at that position. The HD as represented by SEQ ID NO: 234, and comprised in SEQ ID NO: 180, is in accordance with the sequence logo as represented in this figure.

FIG. 16 shows the graphical output of the COILS algorithm predicting two coiled coil domains in the N-terminal half of the polypeptide as represented by SEQ ID NO: 180. The X axis represents the amino acid residue coordinates, the Y axis the probability (ranging from 0 to 1) that a coiled coil domain is present, and the three lines, the three windows (14, 21, 28) examined.

FIG. 17 shows a CLUSTAL W (1; 83) multiple sequence alignment of PHDf-HD polypeptides from Table D1 (when full length), where a number of features are identified. From the N-terminus to the C-terminus of the polypeptides are: (i) a predicted nuclear localisation signal (NLS); (ii) a leucine zipper (ZIP), with four heptads (boxed, in which usually a leucine (occasionally an isoleucine, a valine, or a methionine) appears every seventh amino acid); (iii) a PHD finger (PHDf), with the typical C4HC3 (four cysteines, one histidine, three cysteines) with a characteristic cysteine spacing; (iv) an acidic stretch (rich in acidic amino acids D and E); (v) basic stretches (rich in basic amino acids K and R); (vi) a homeodomain (HD).

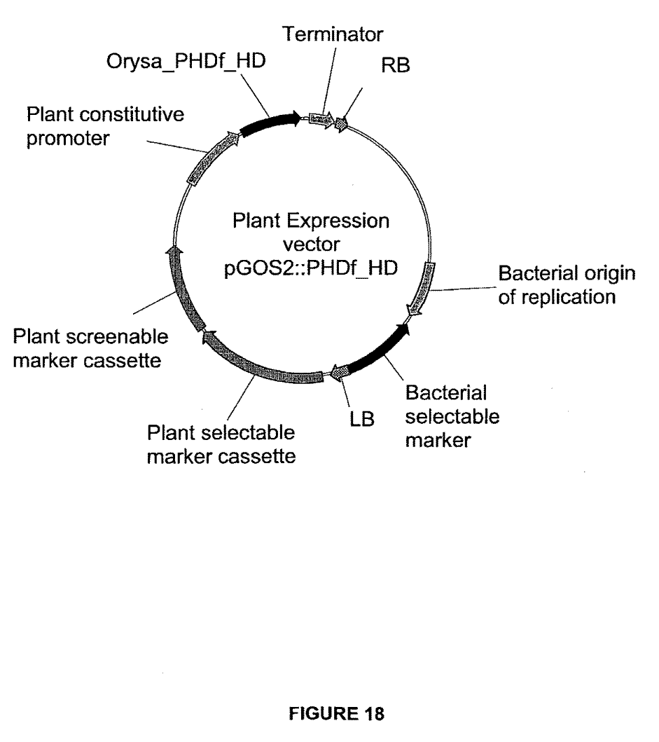

FIG. 18 shows the binary vector for modulated, preferably increased, expression in Oryza sativa of a nucleic acid sequence encoding a PHDf-HD polypeptide under the control of a rice GOS2 promoter (pGOS2)

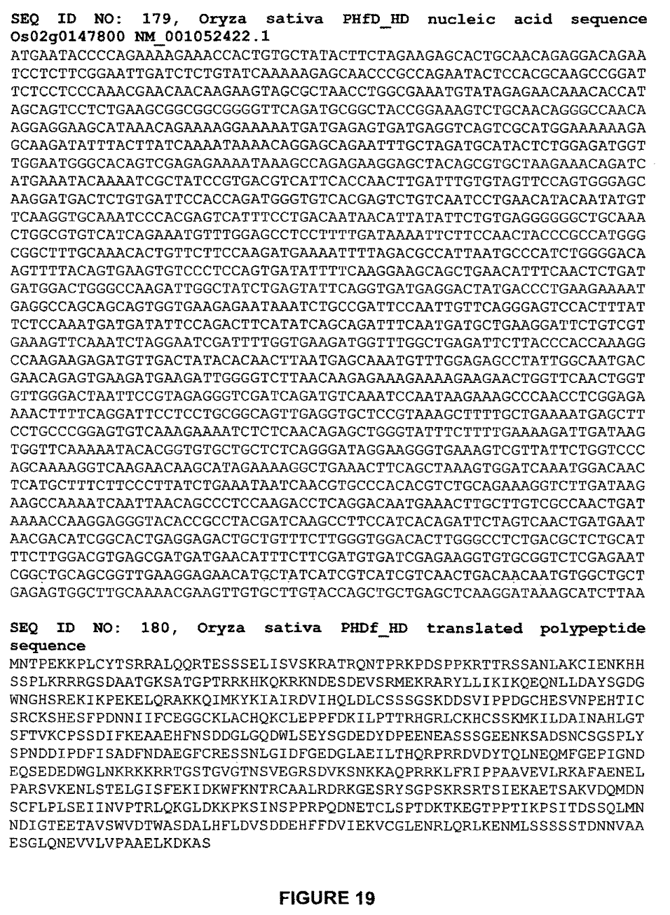

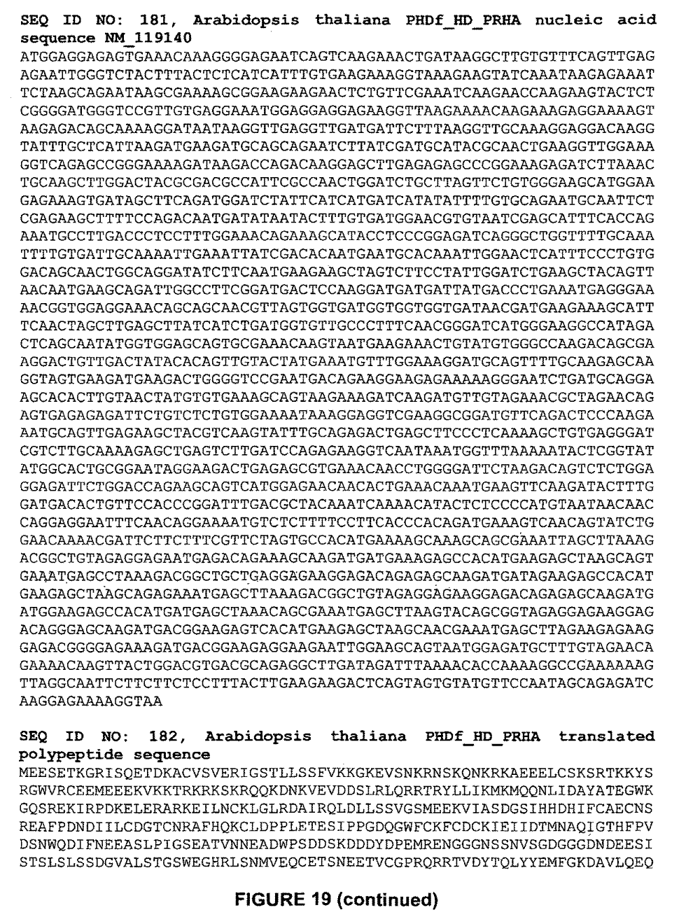

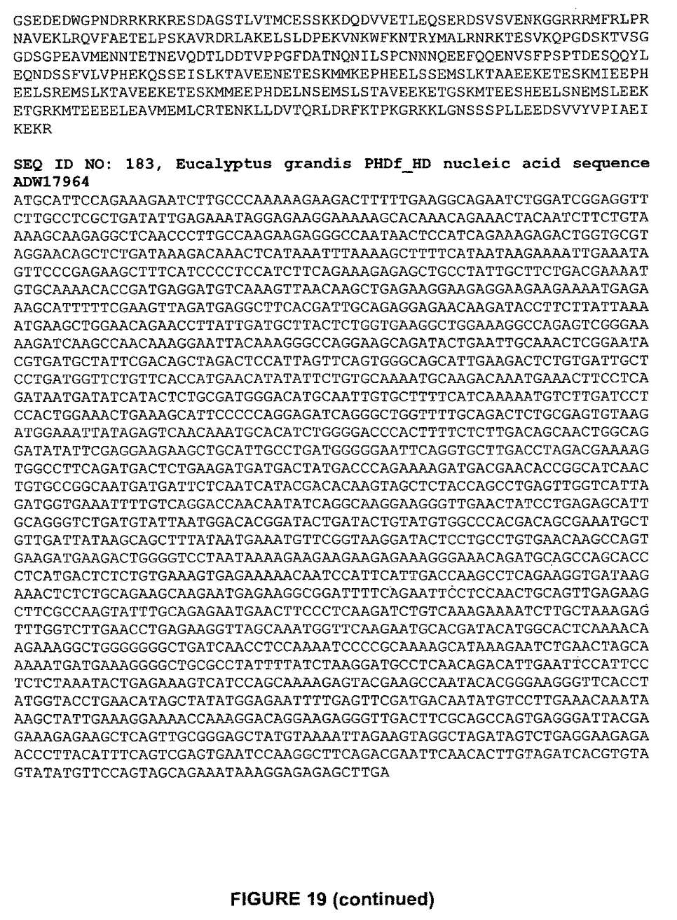

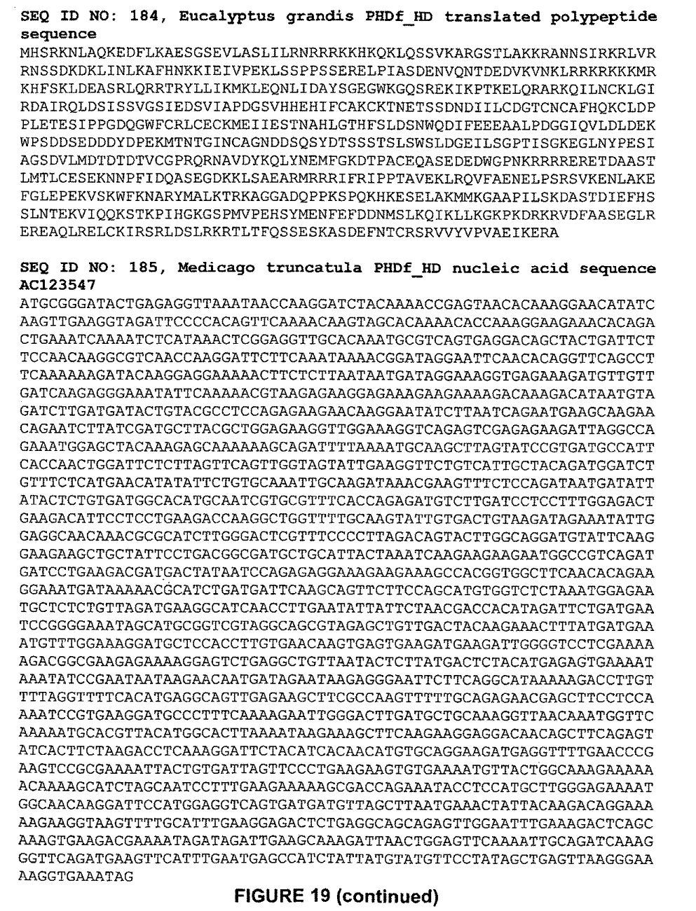

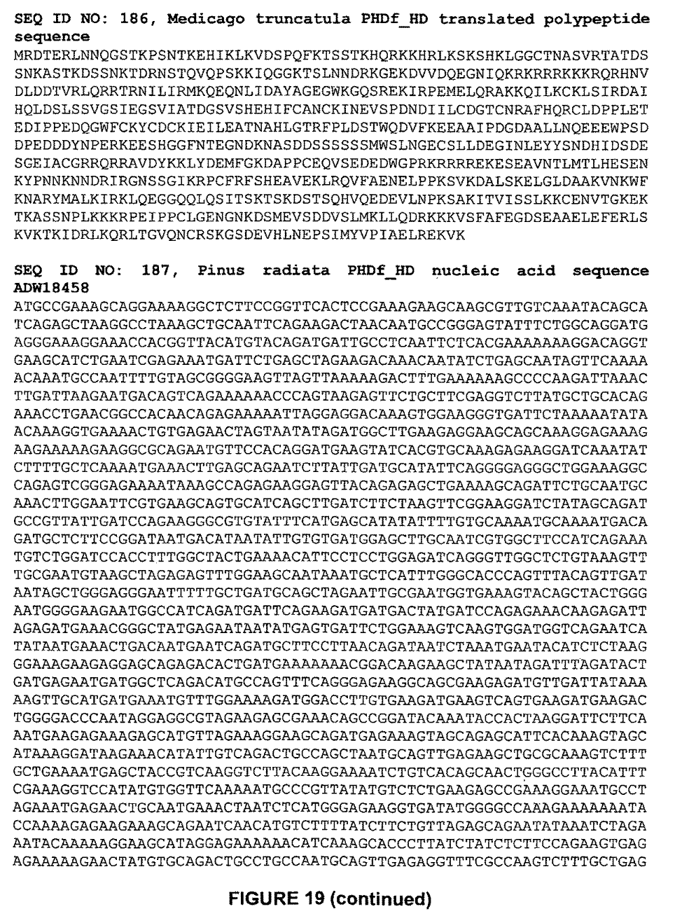

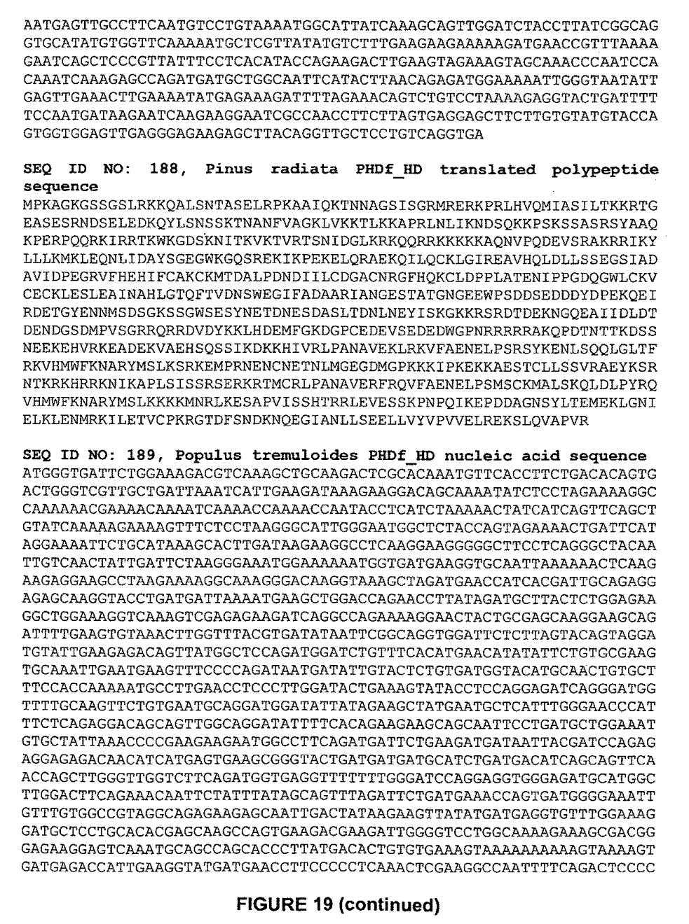

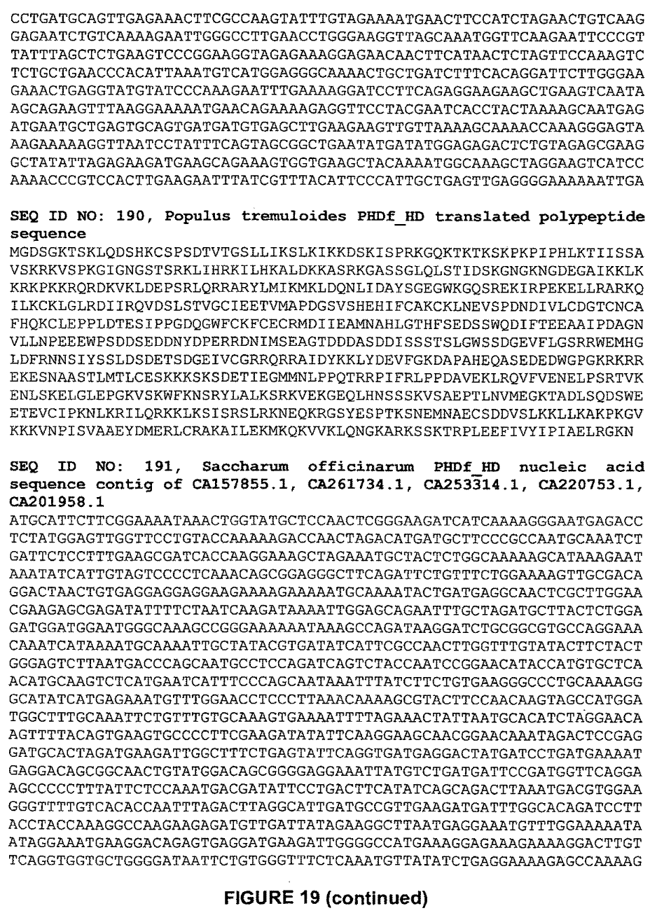

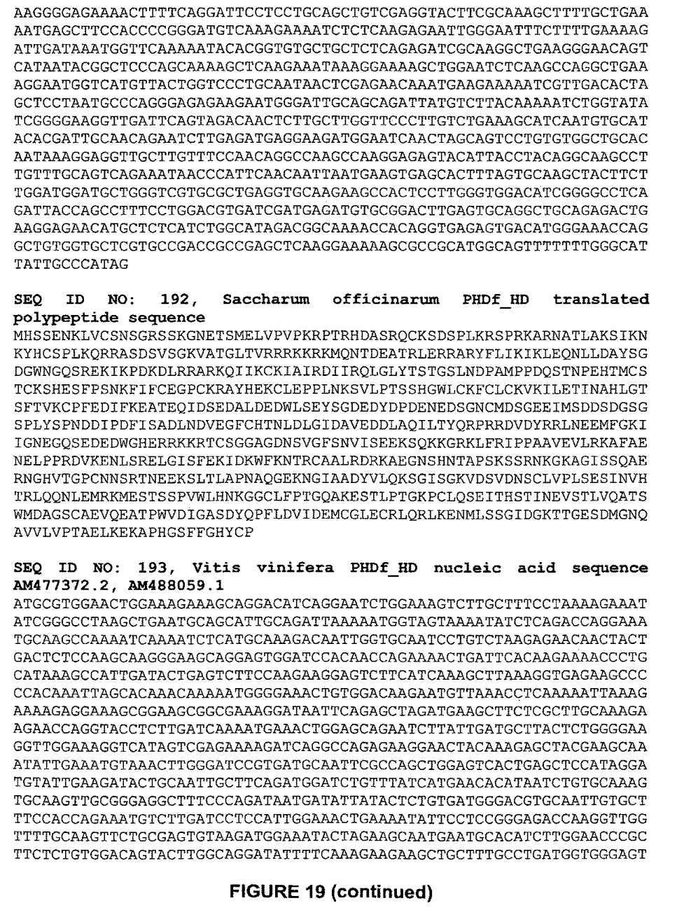

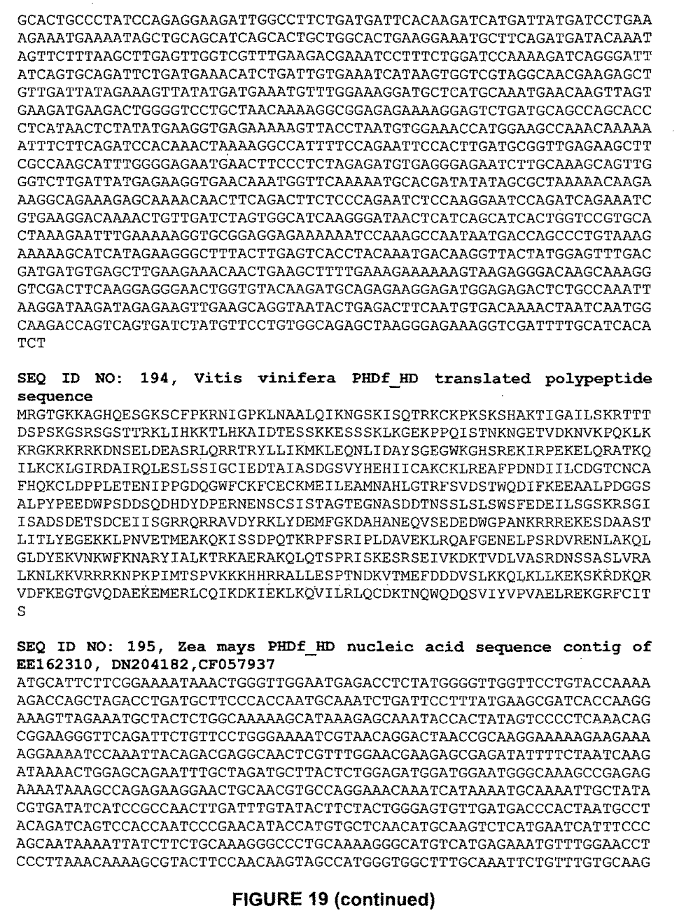

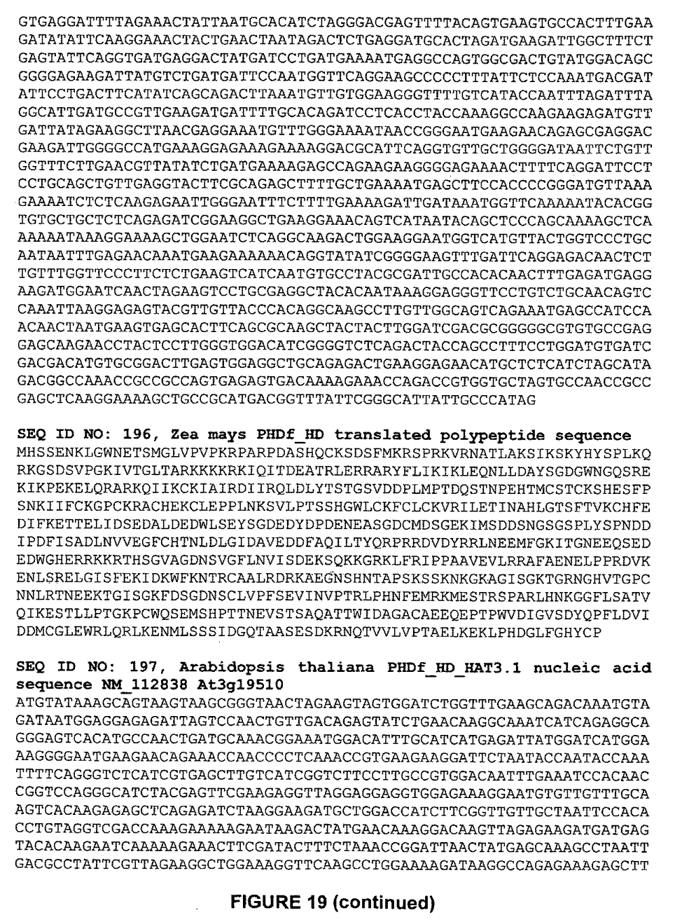

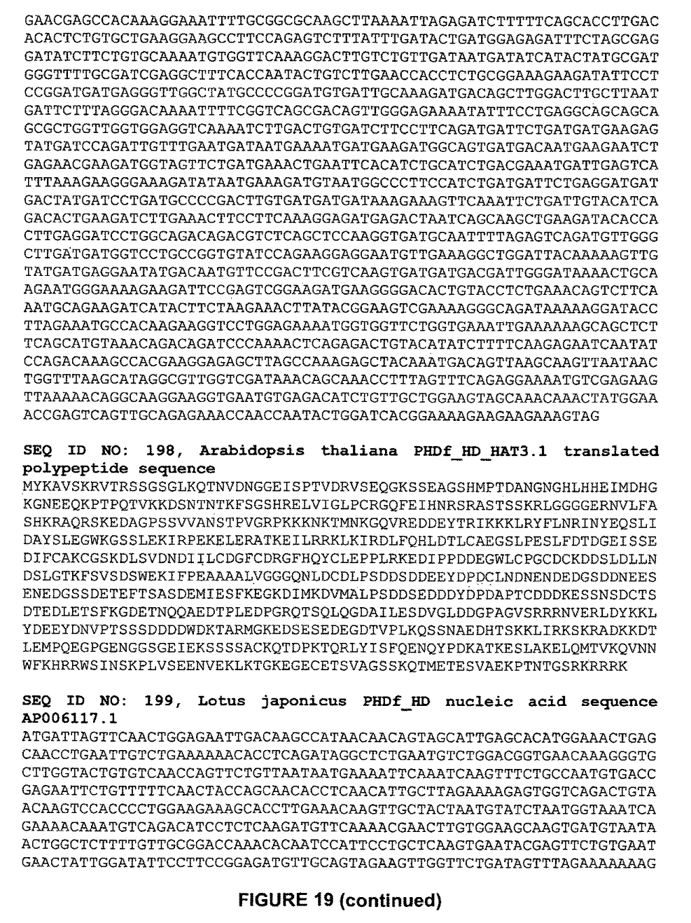

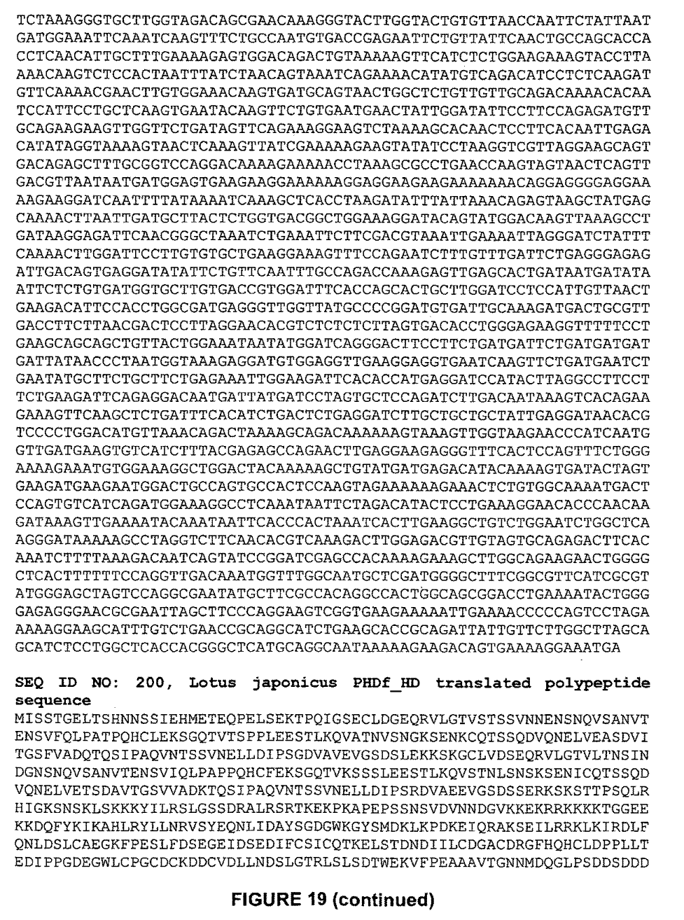

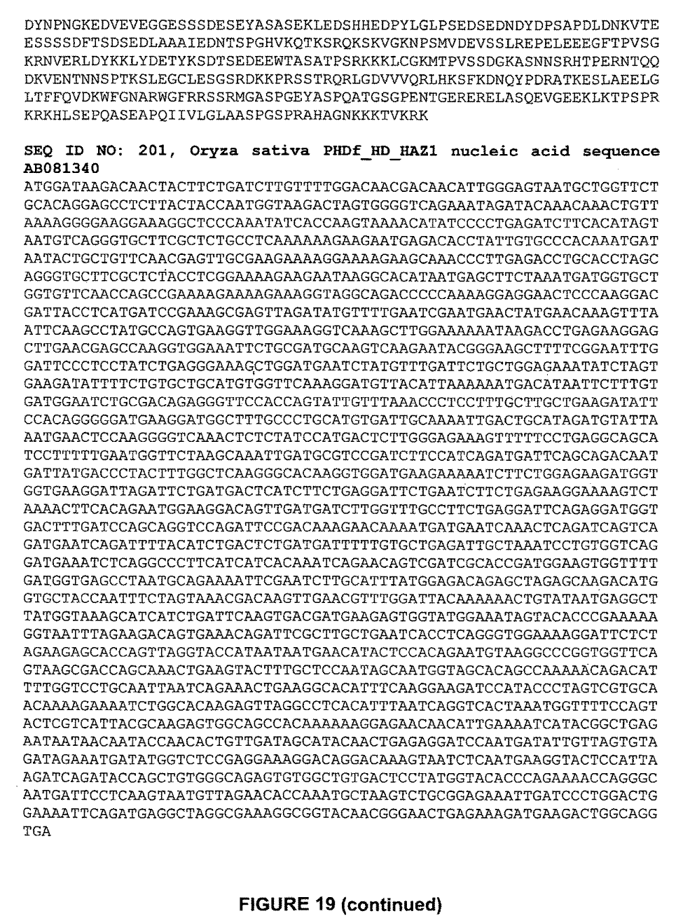

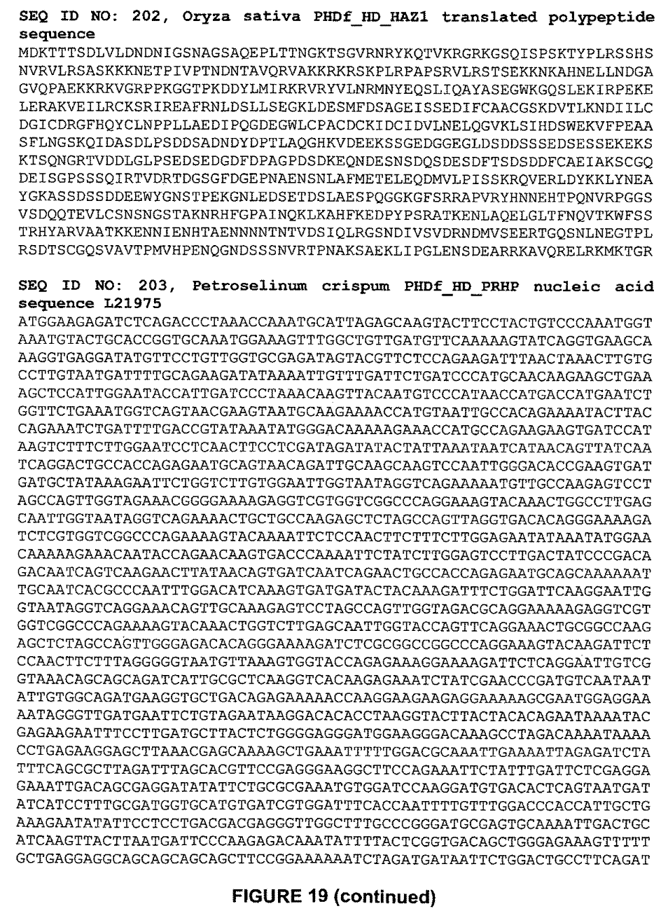

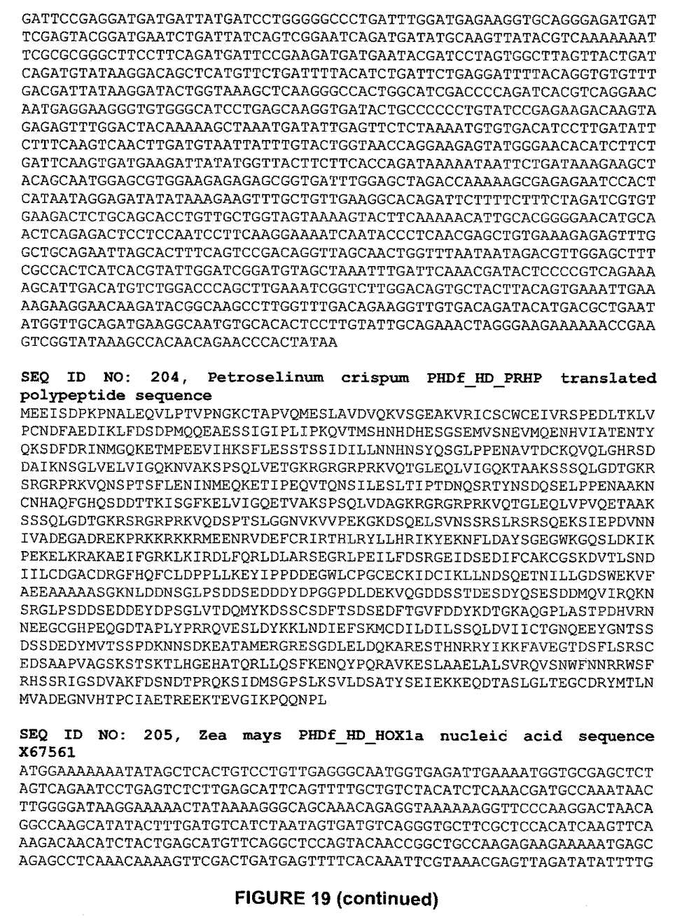

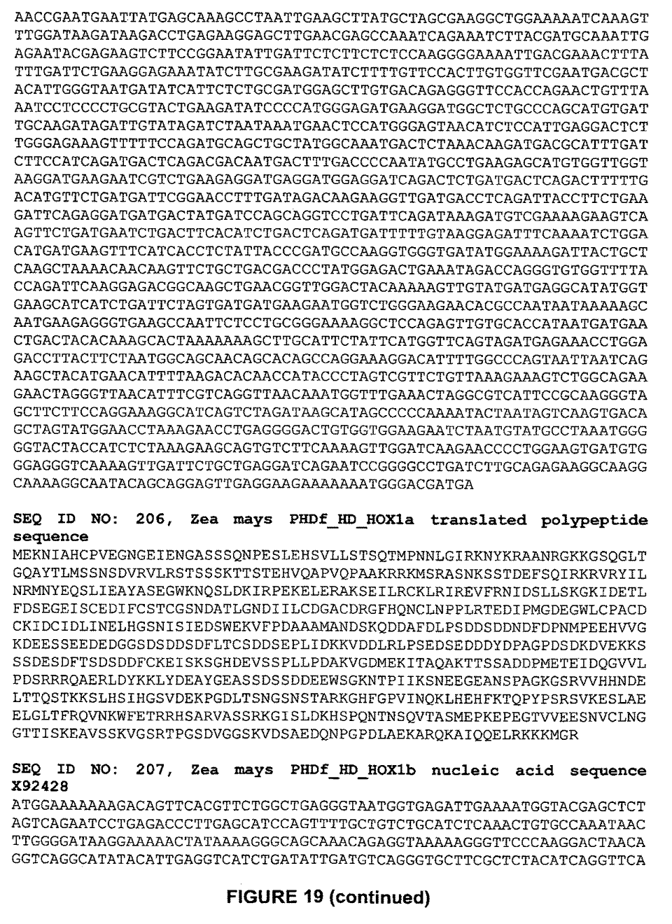

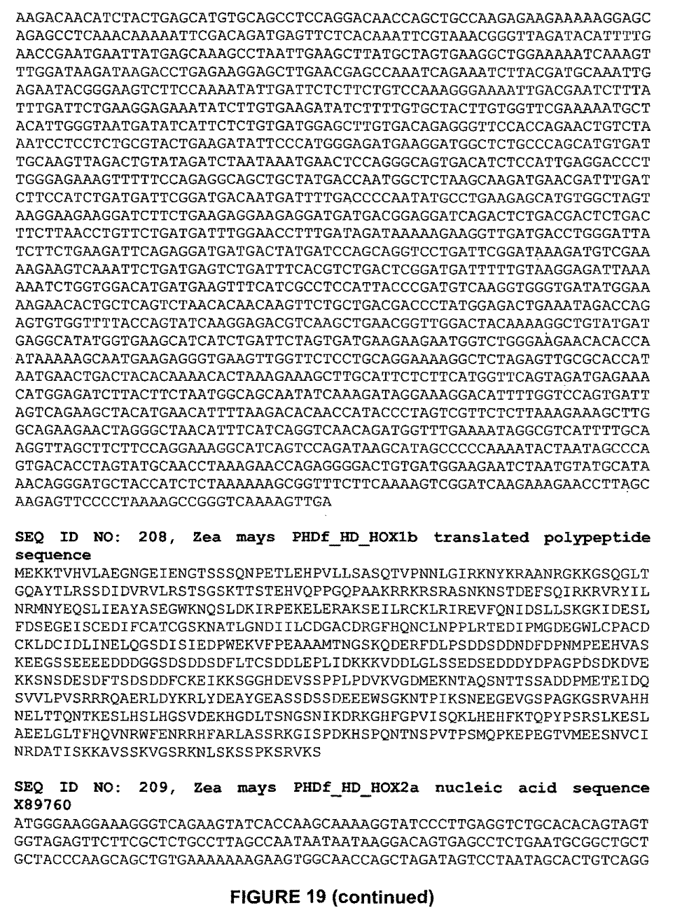

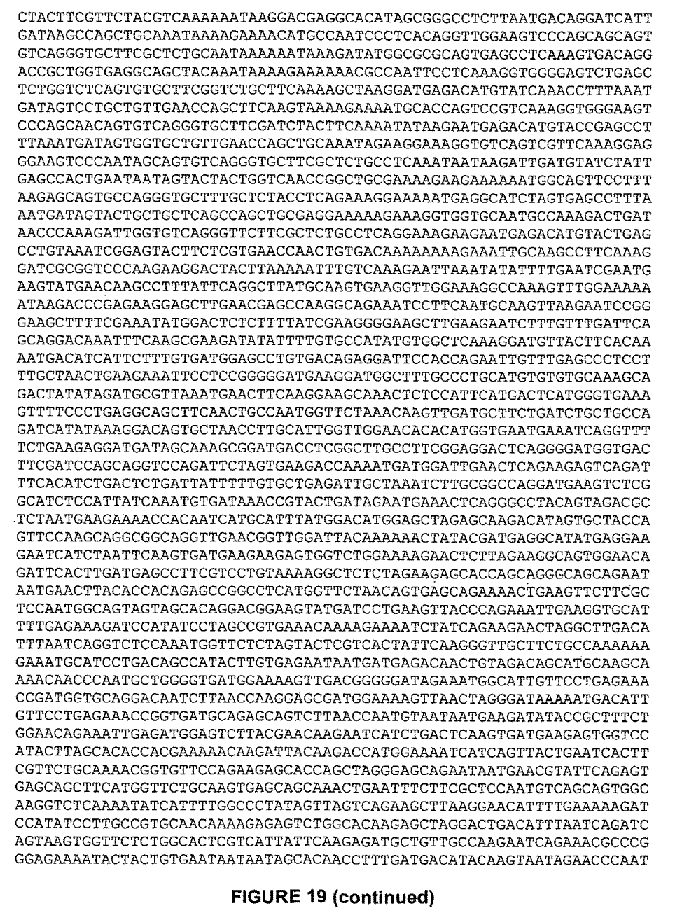

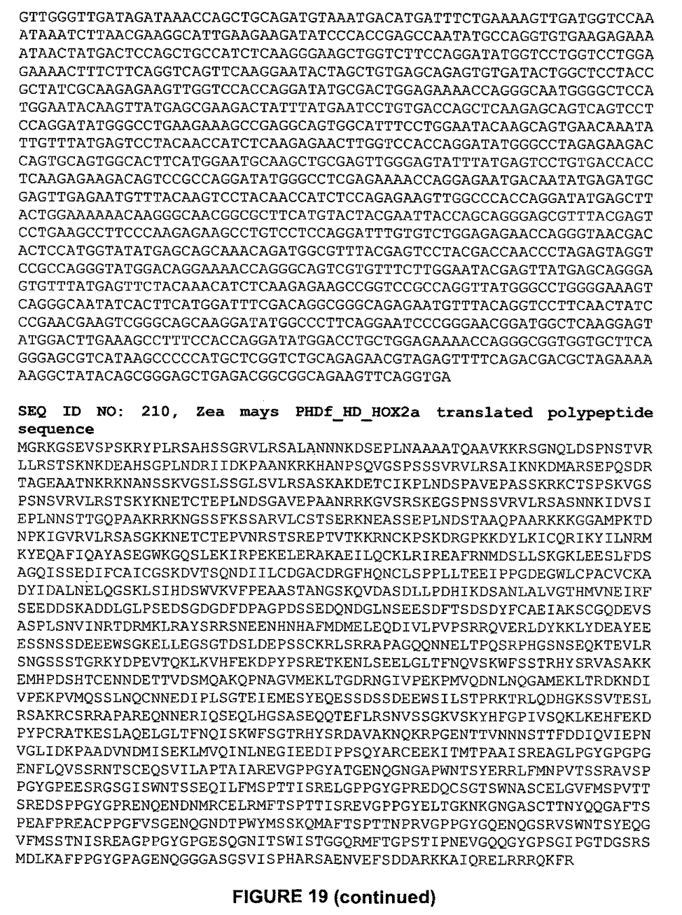

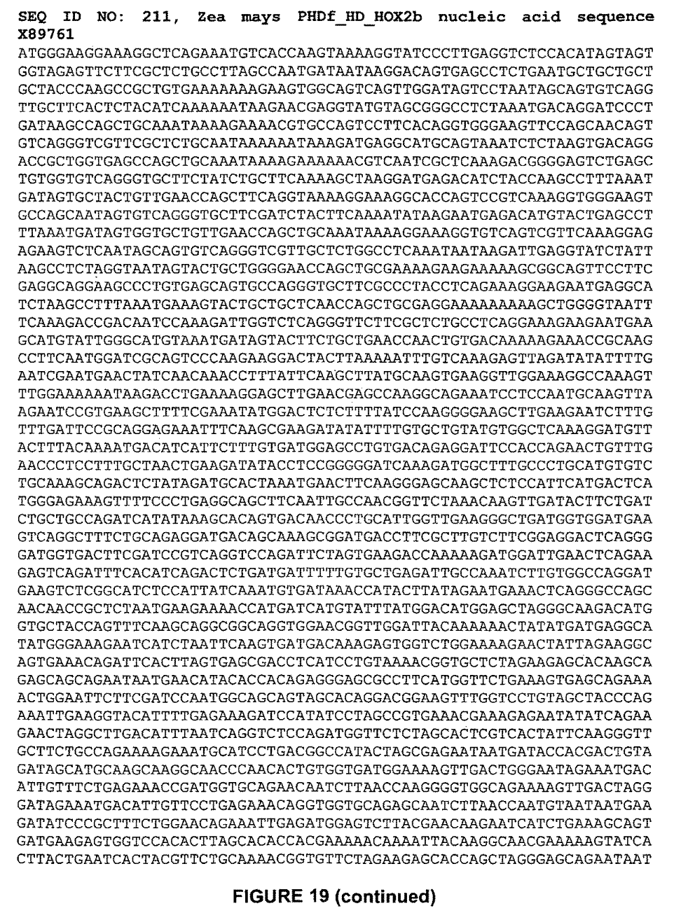

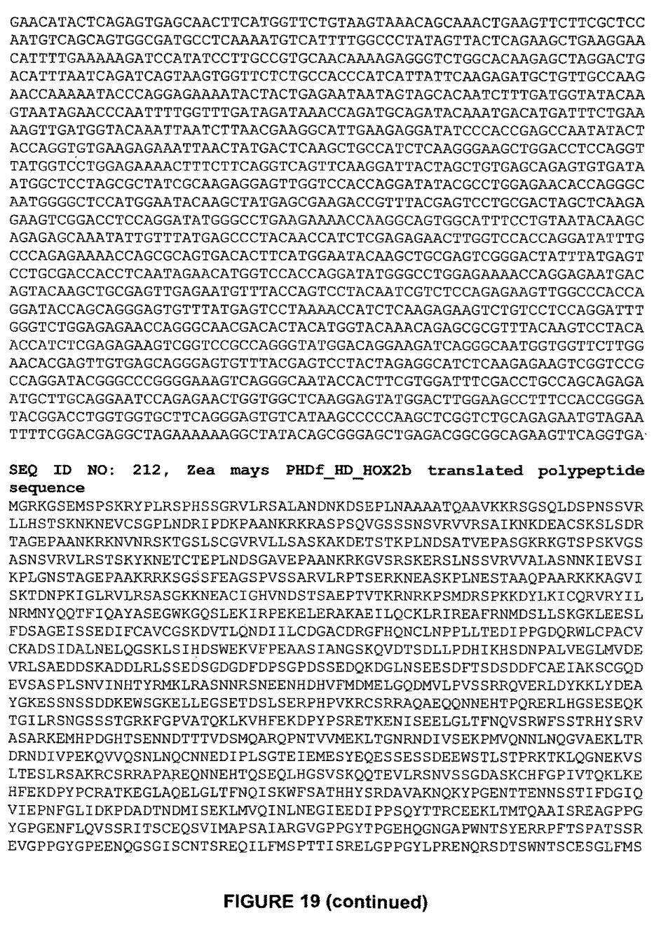

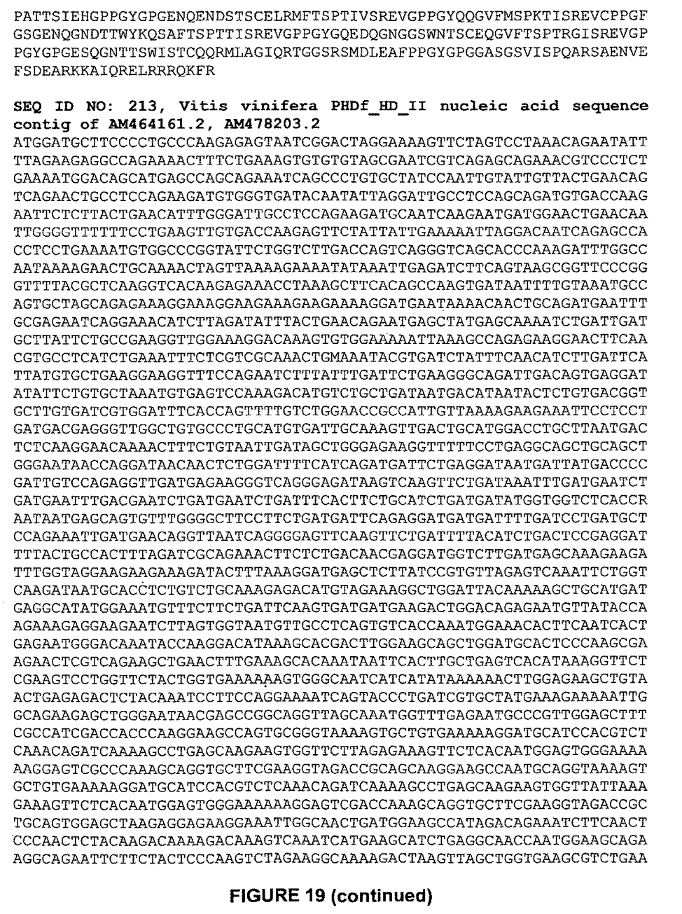

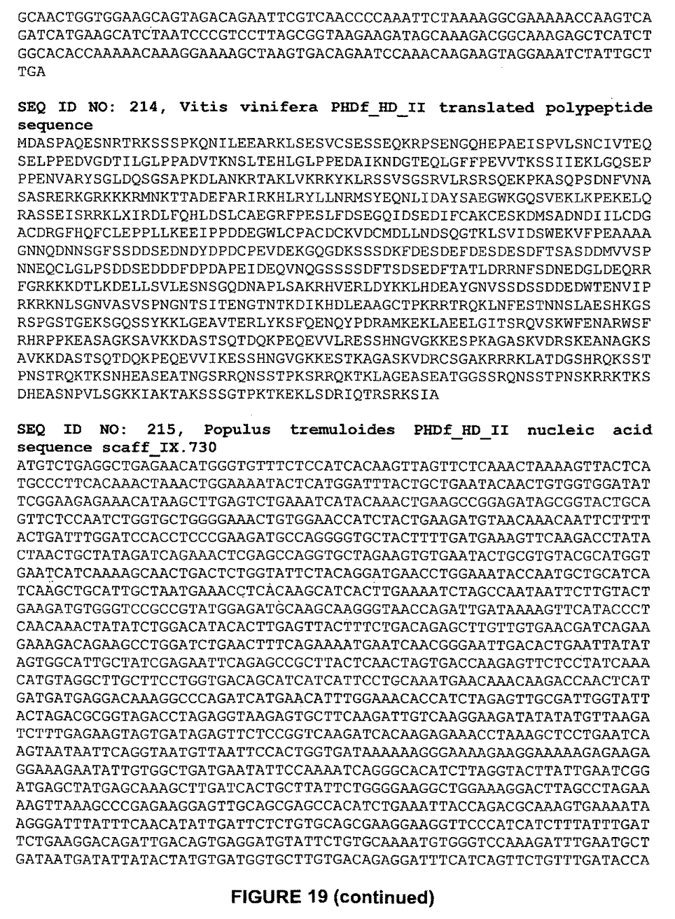

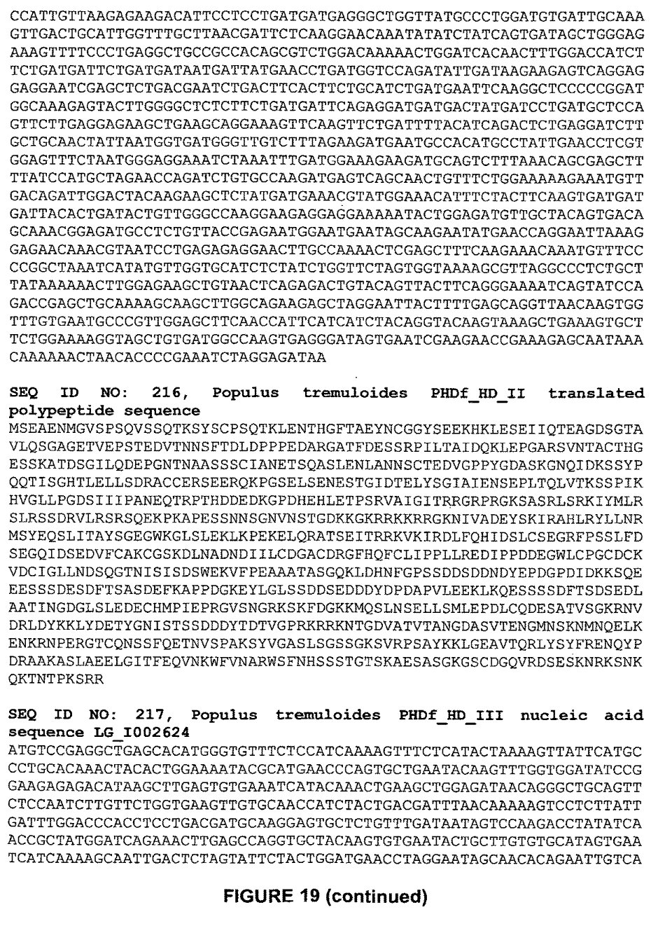

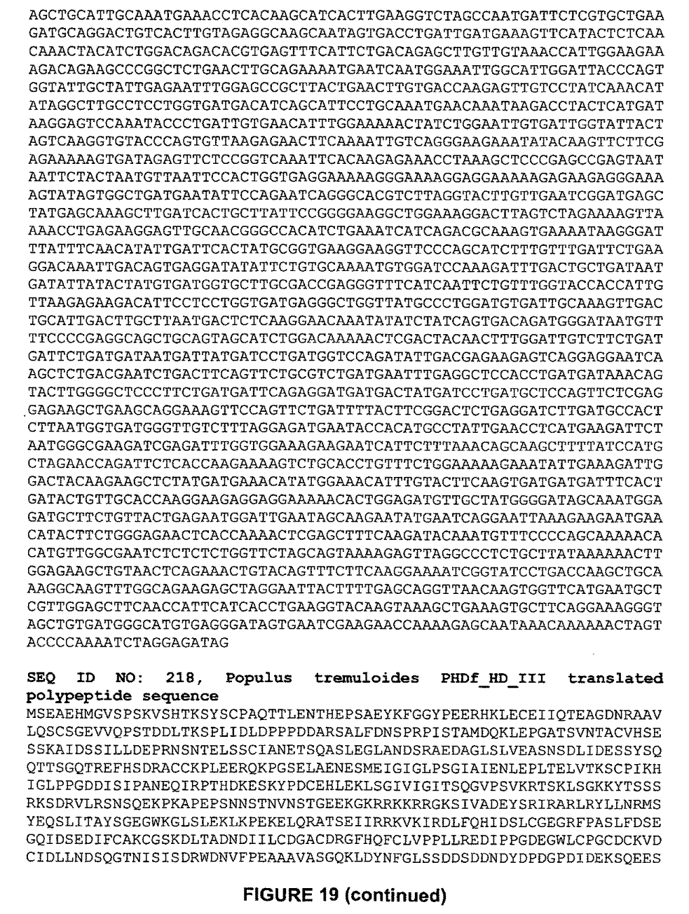

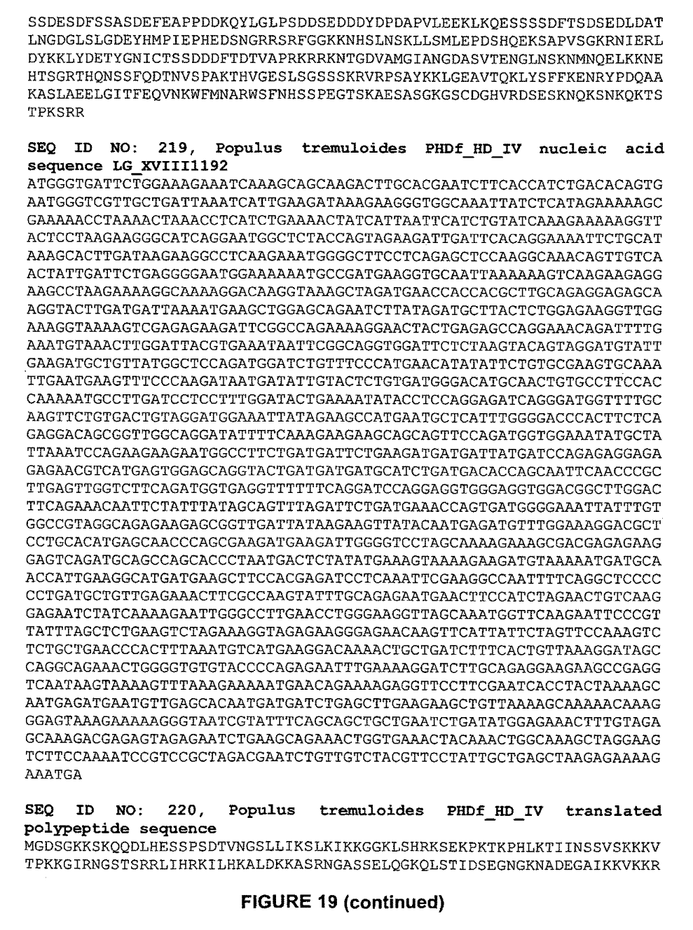

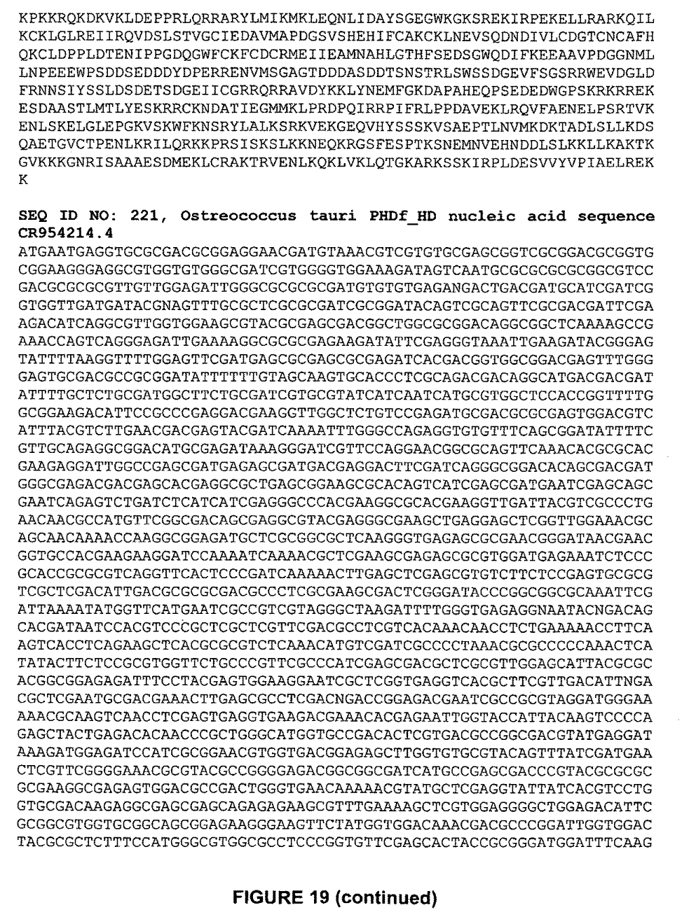

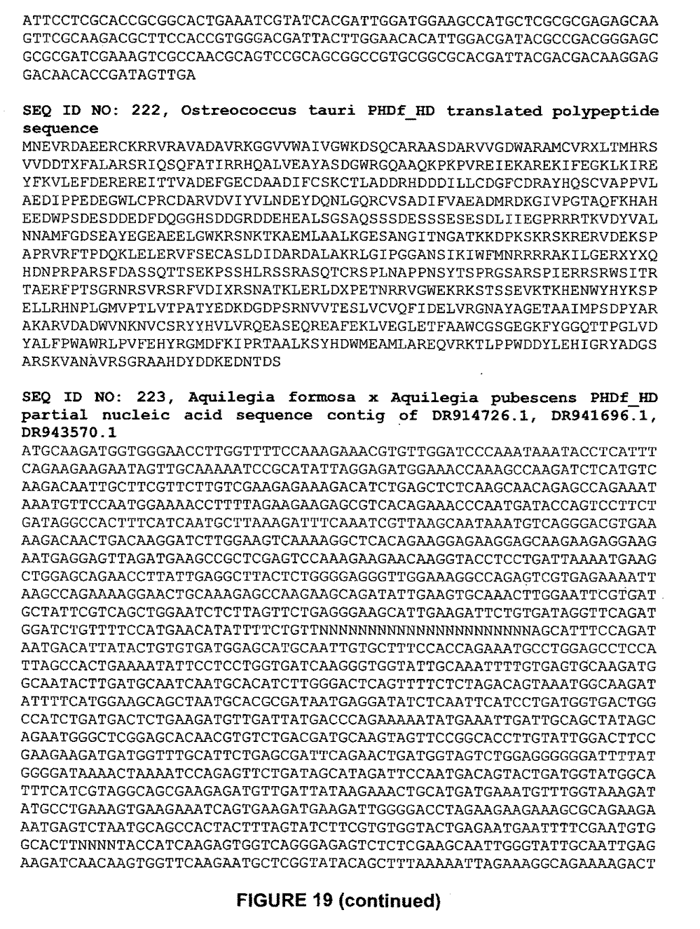

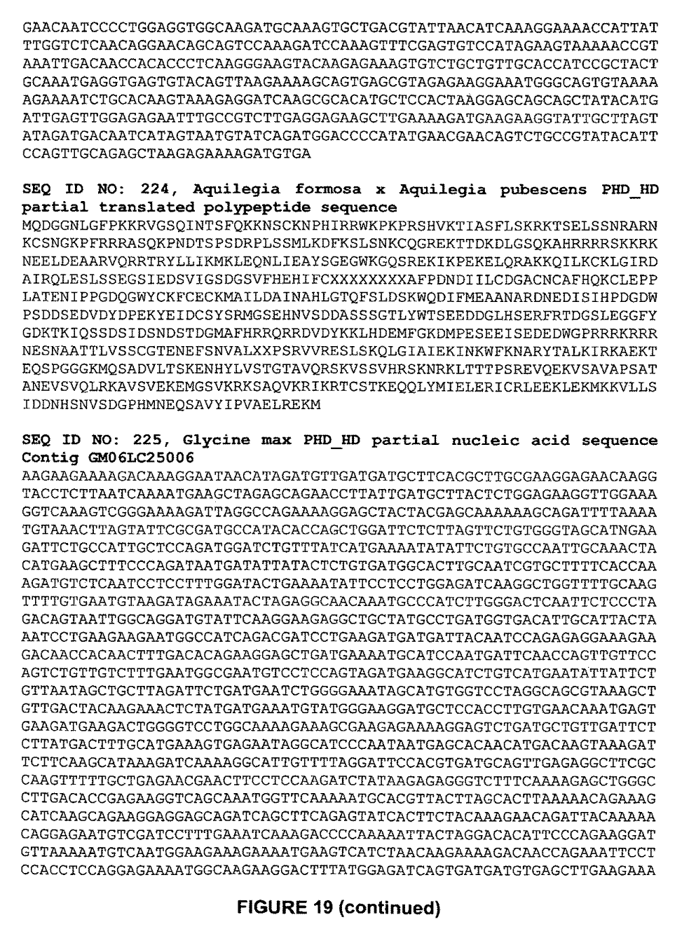

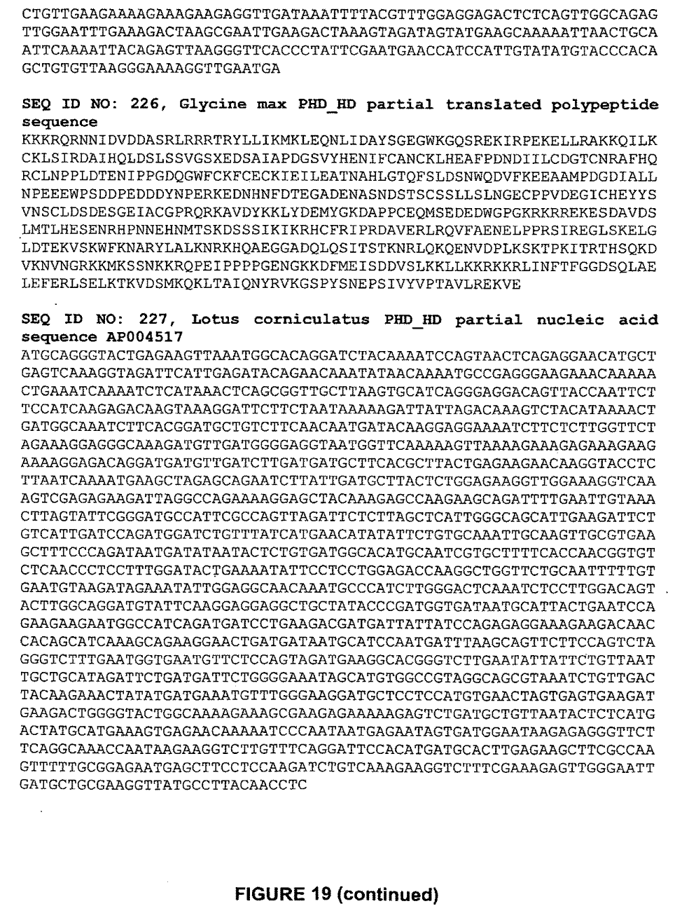

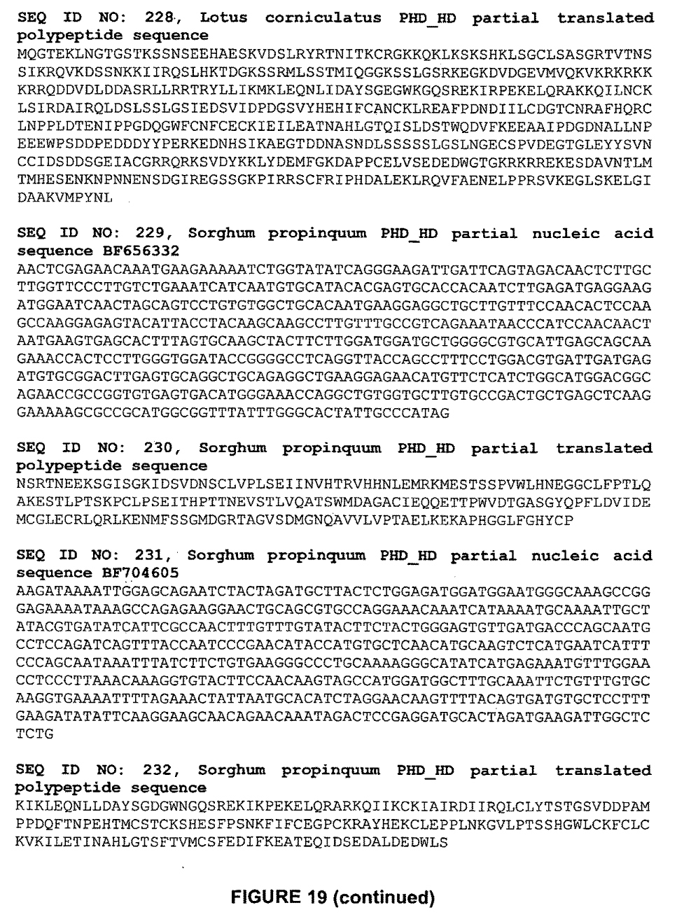

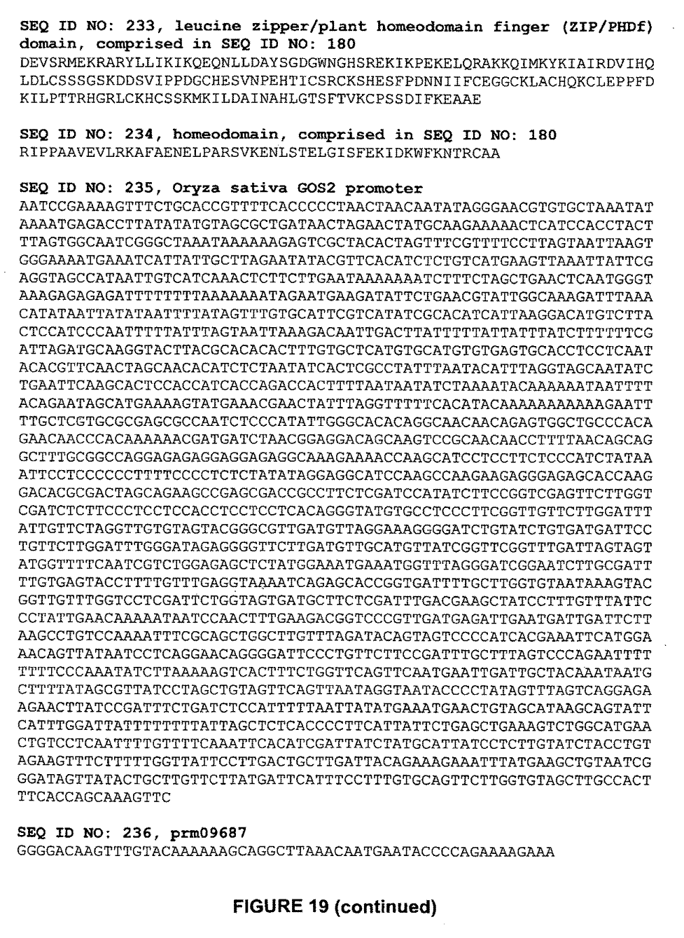

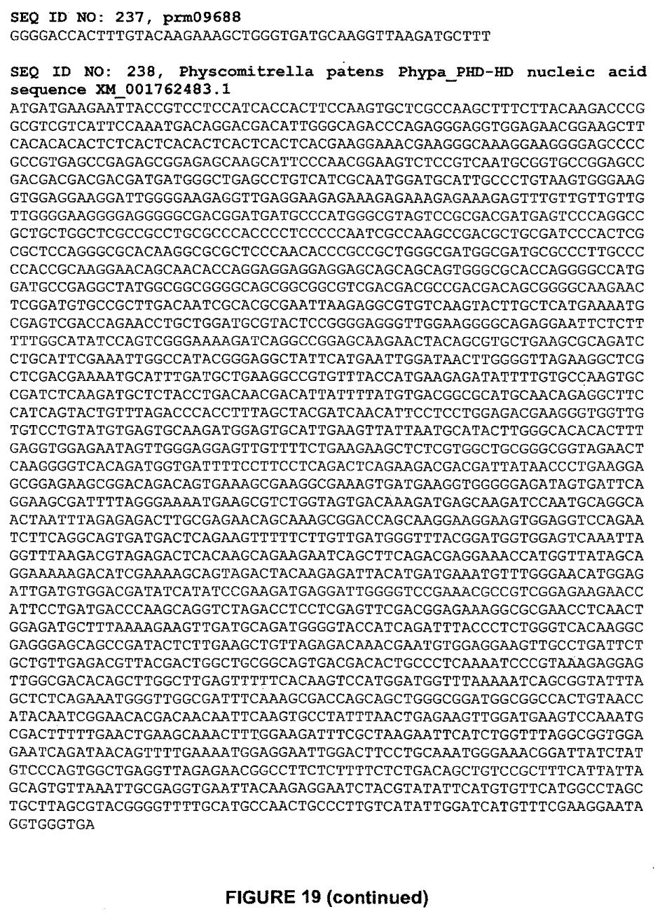

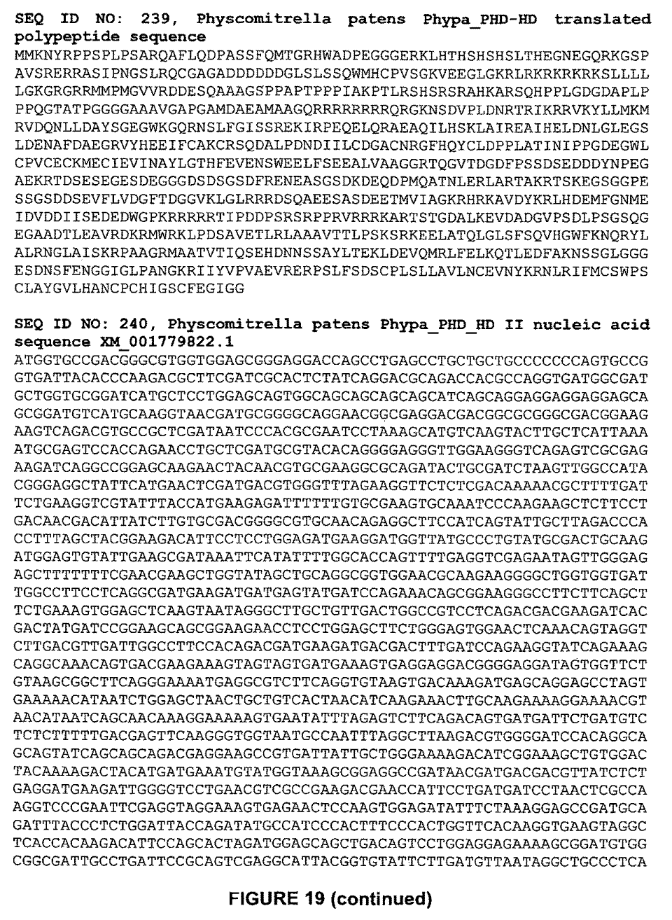

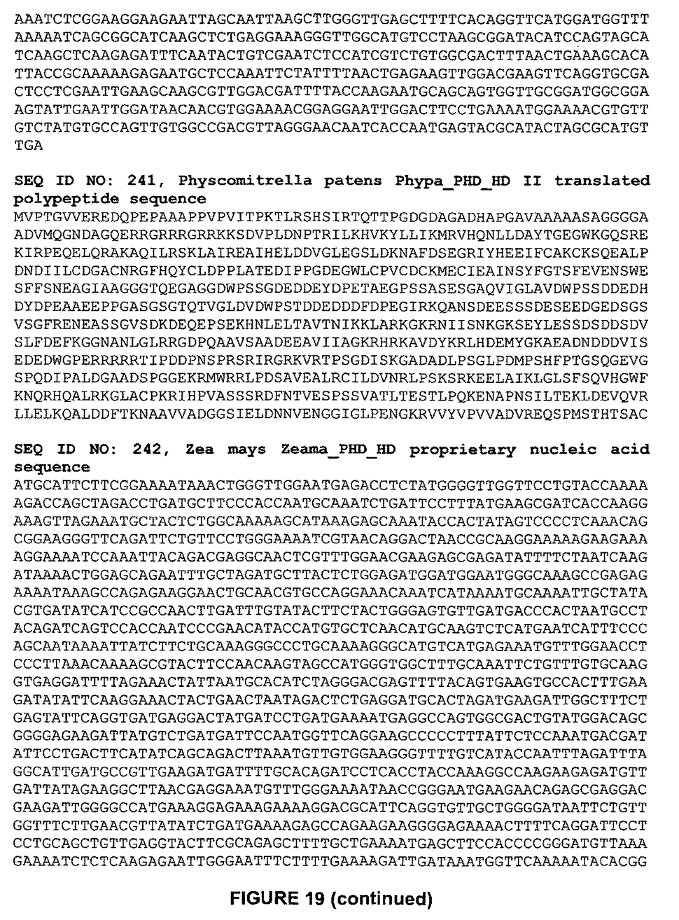

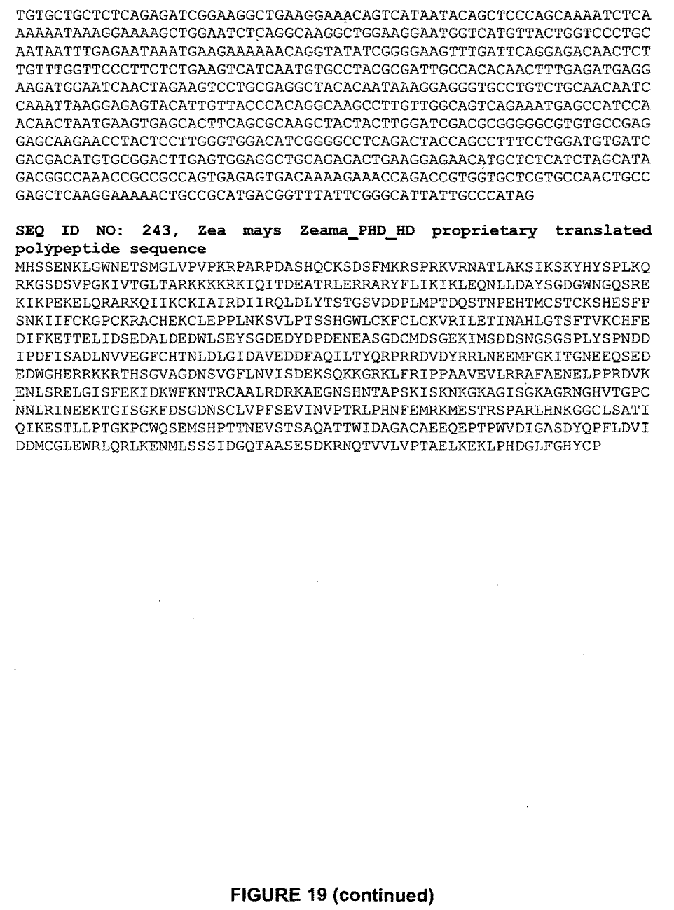

FIG. 19 details examples of a PHDf-HD sequences useful in performing the methods according to the present invention.

V. bHLH11-Like (Basic Helix-Loop-Helix 11) Protein

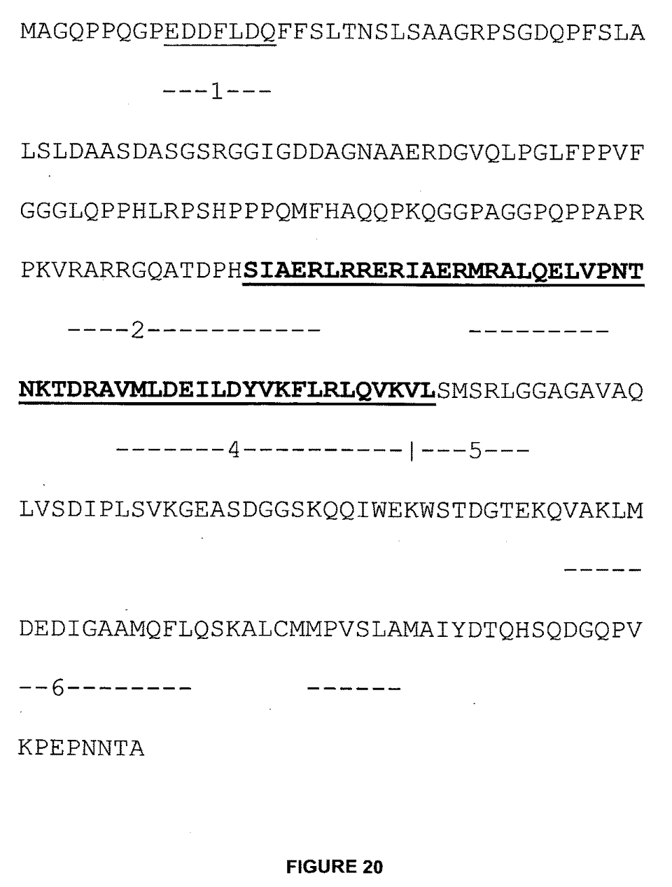

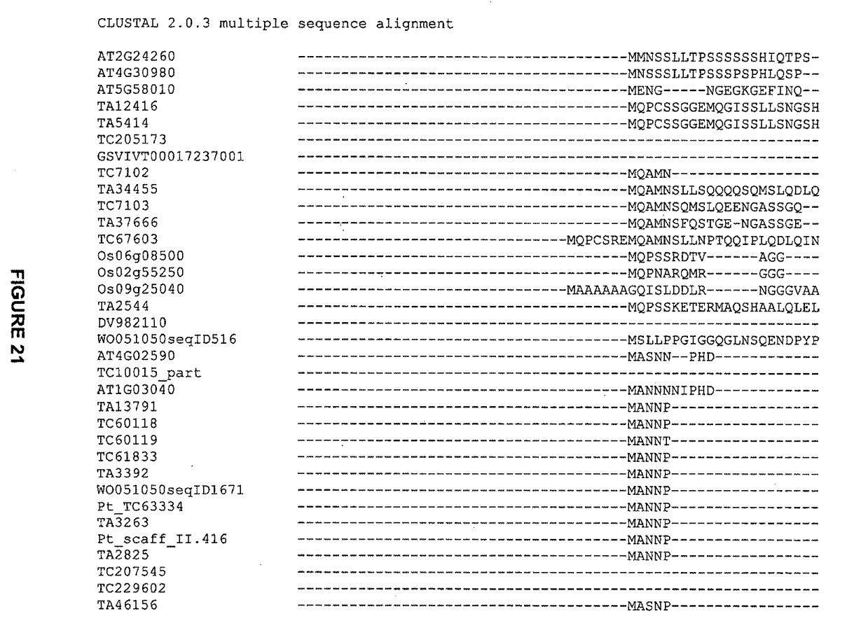

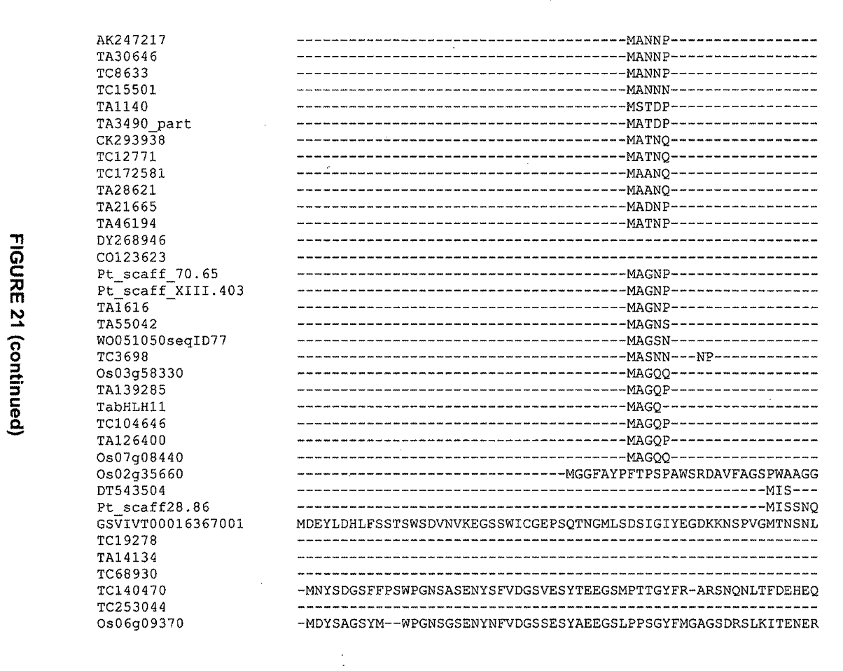

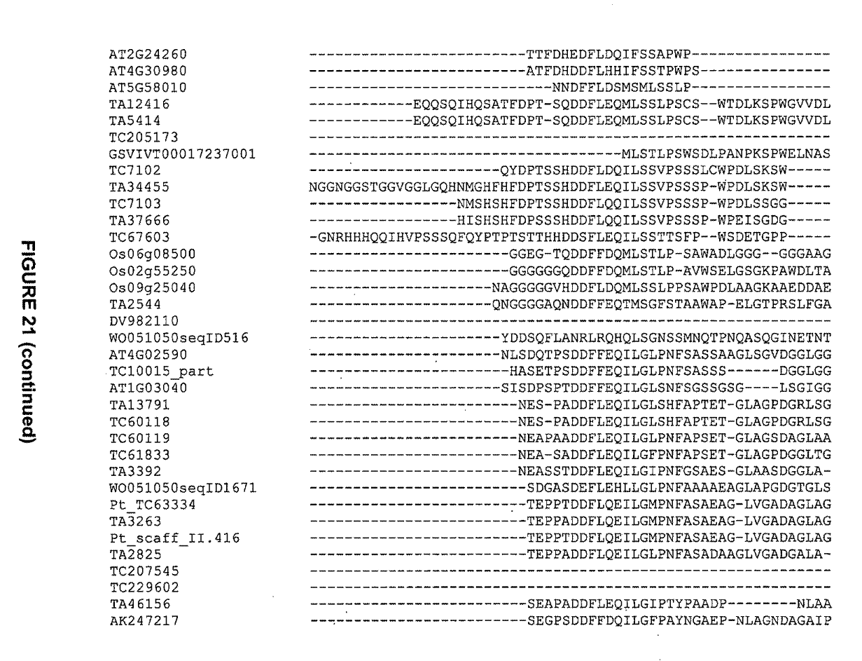

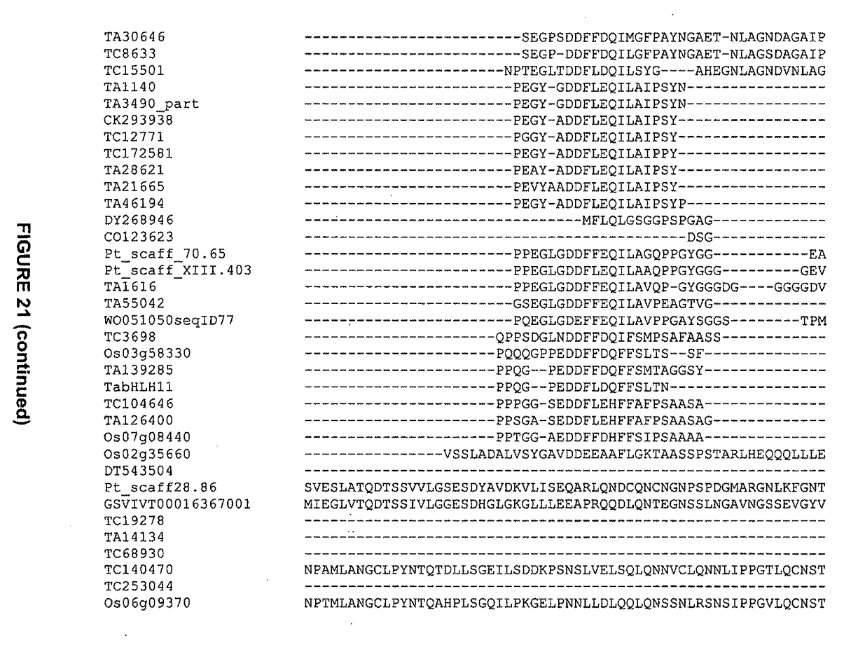

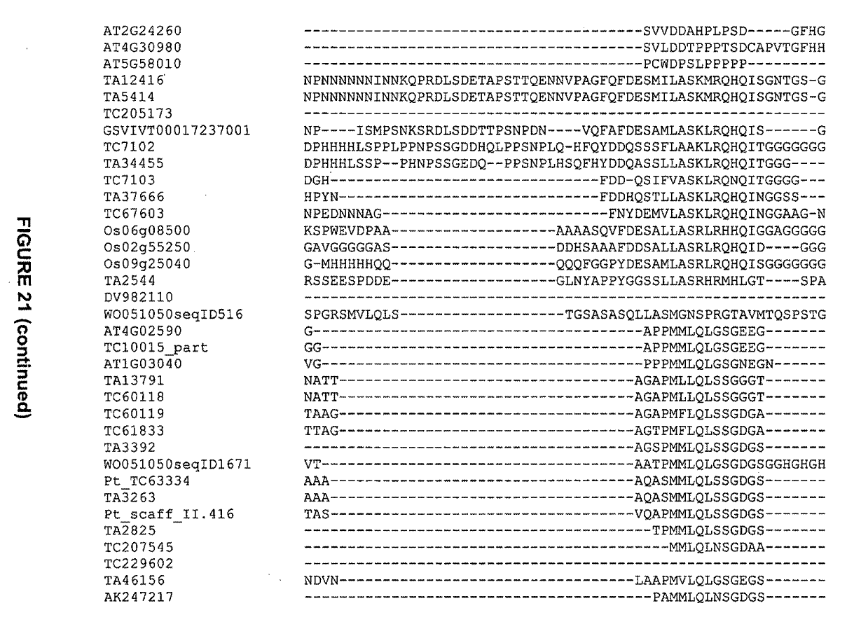

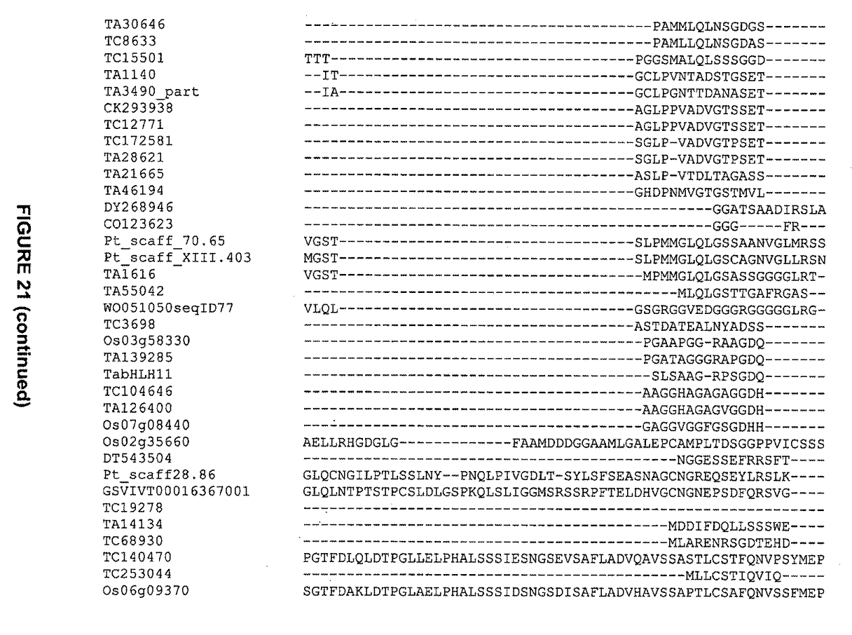

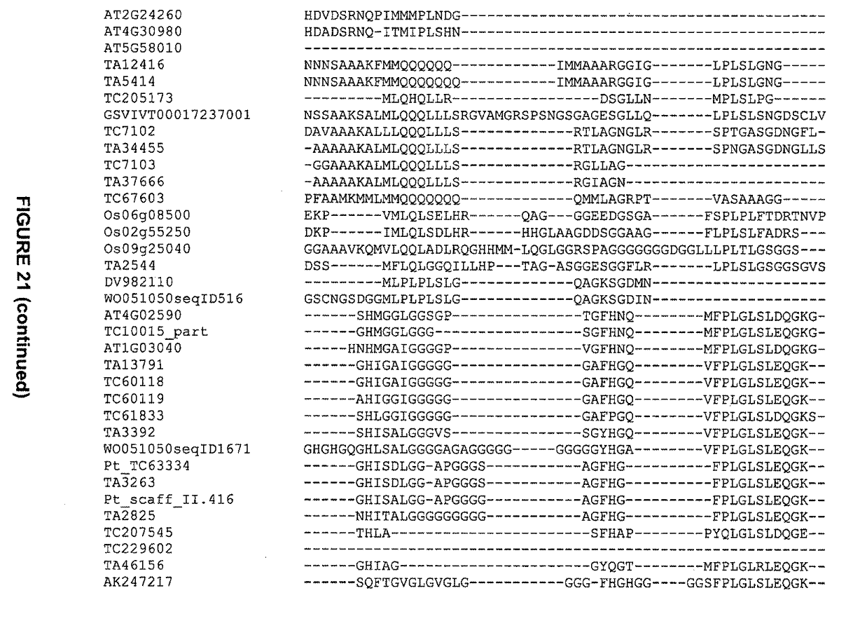

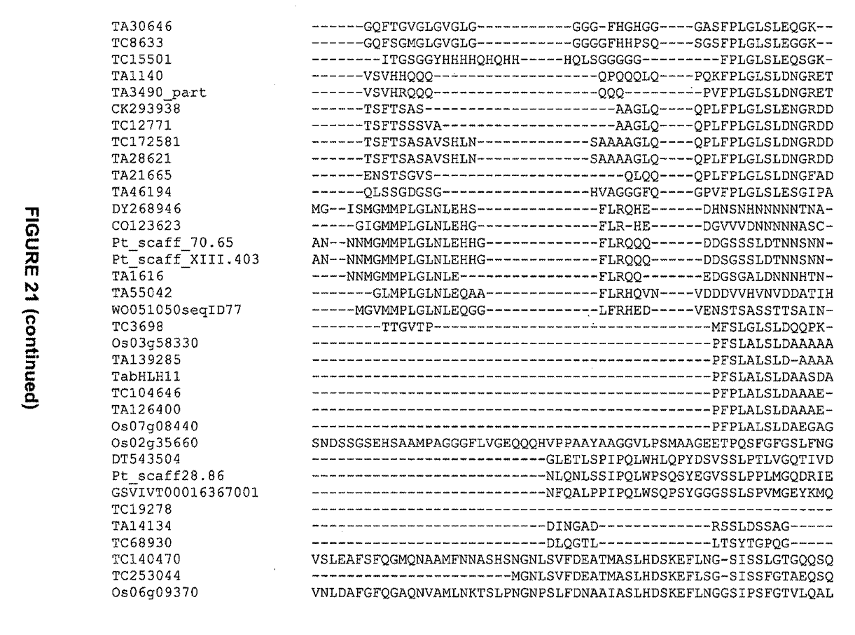

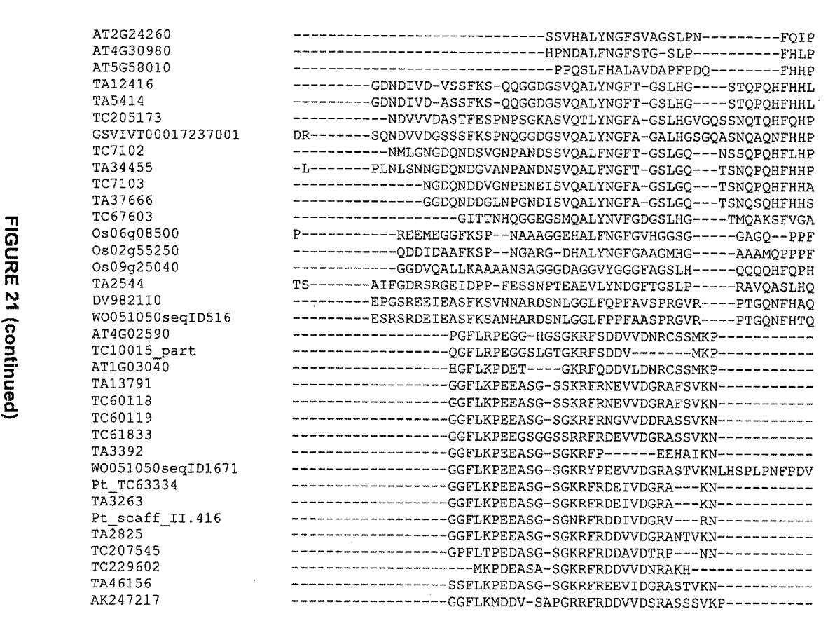

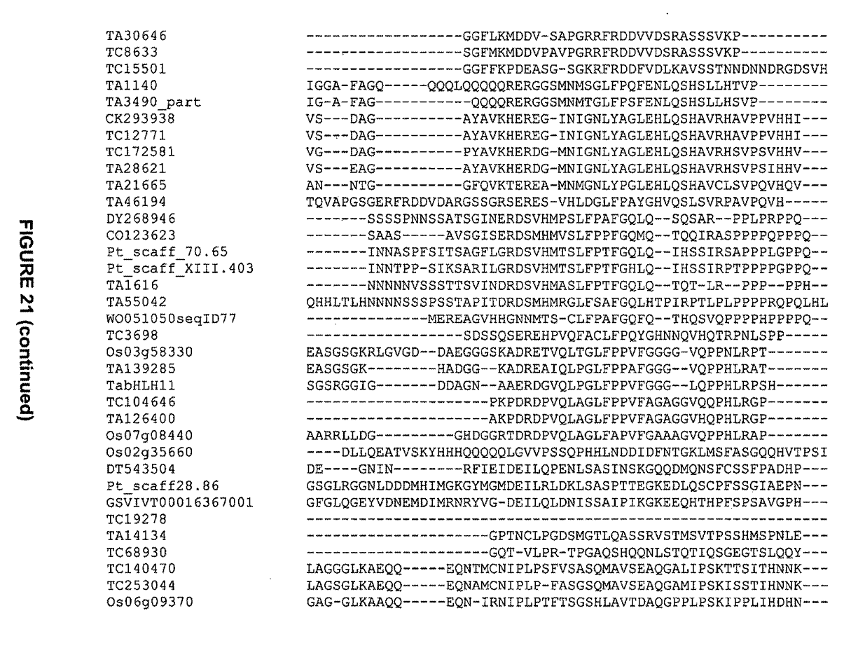

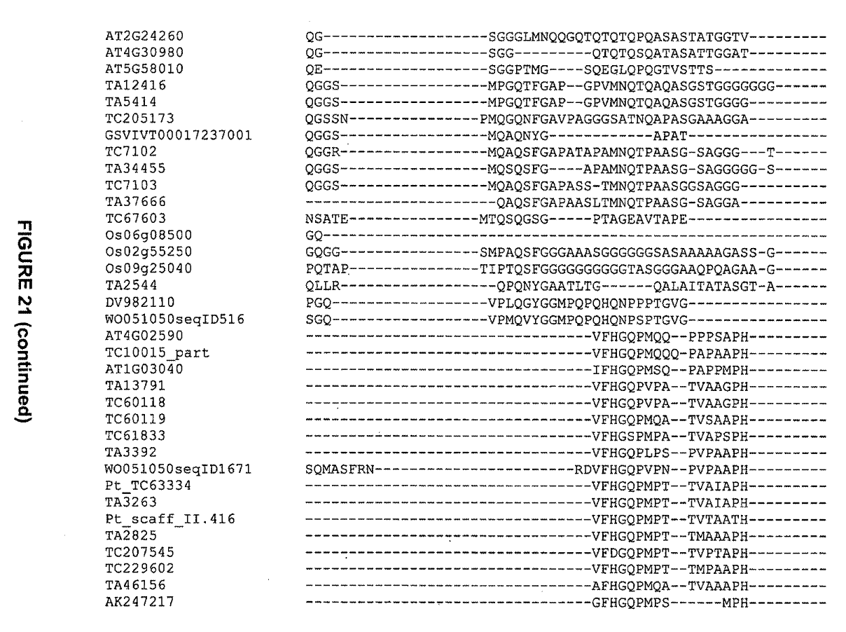

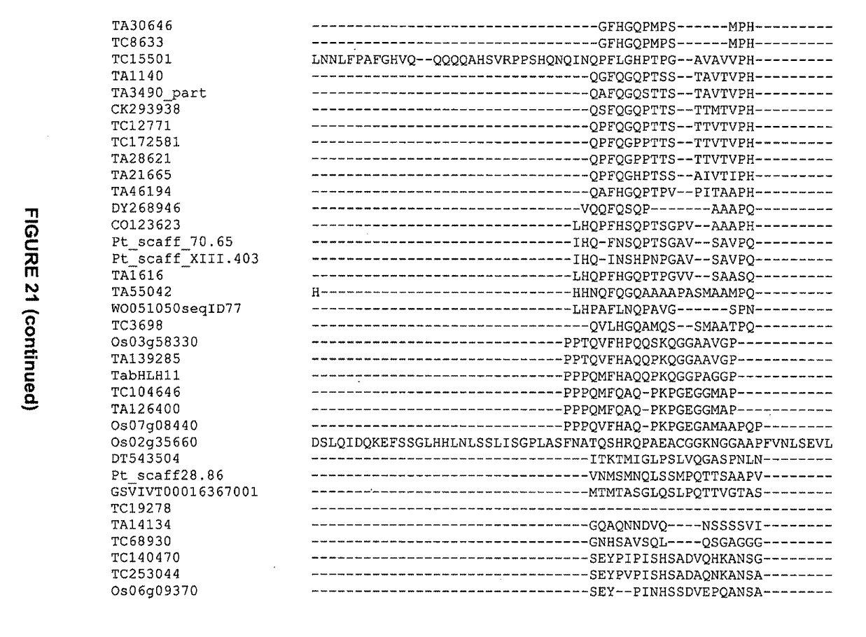

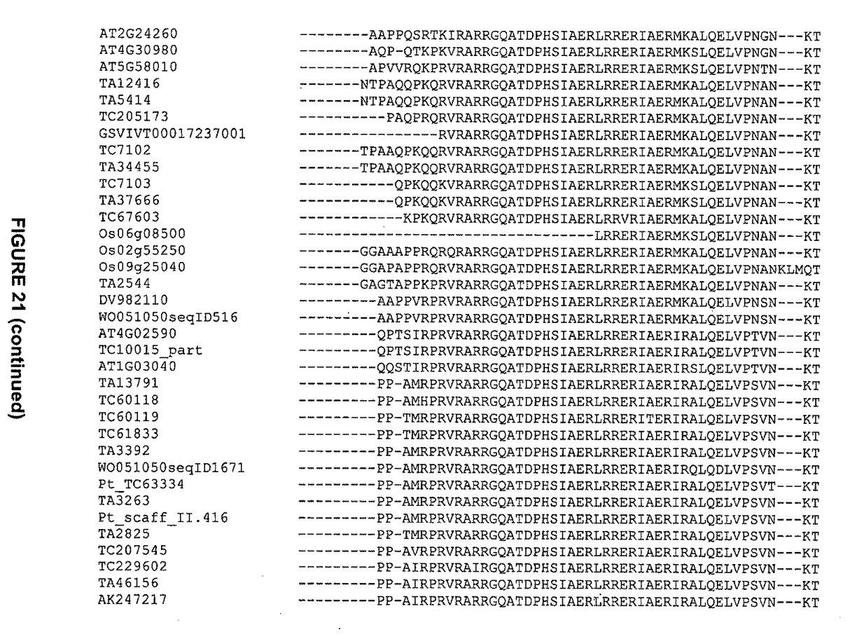

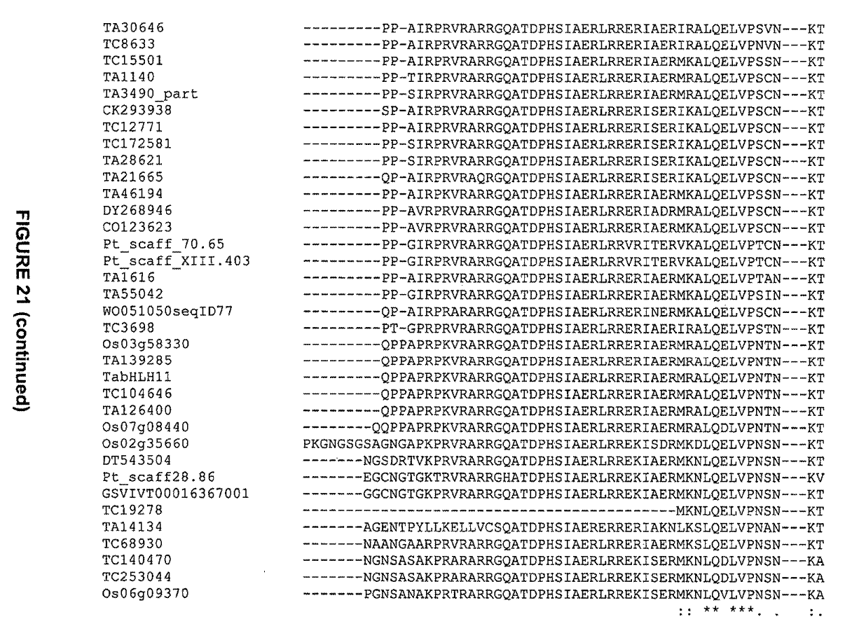

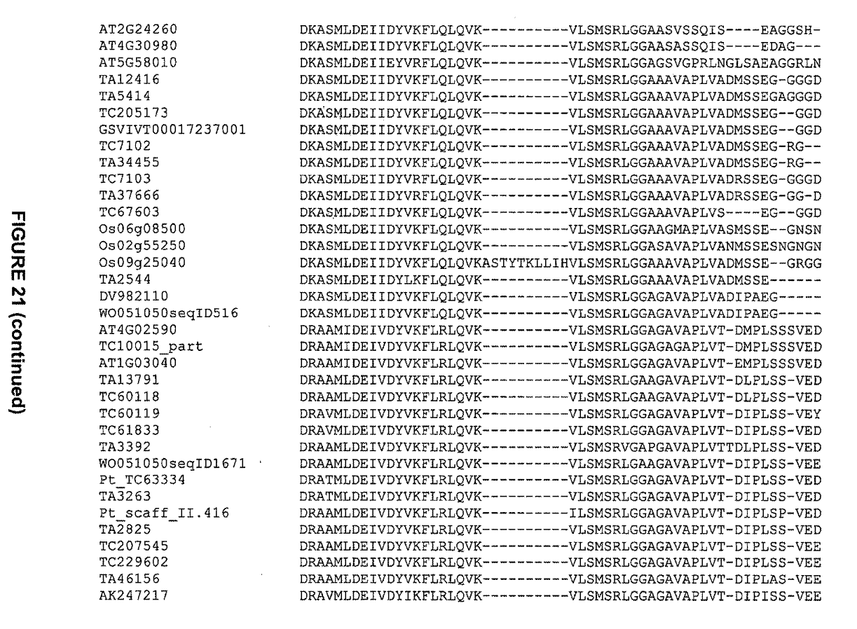

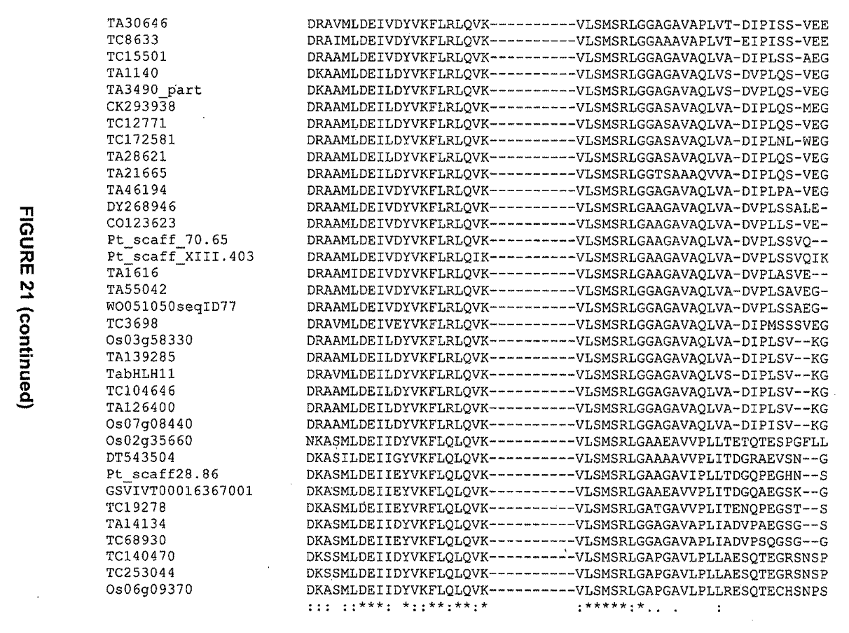

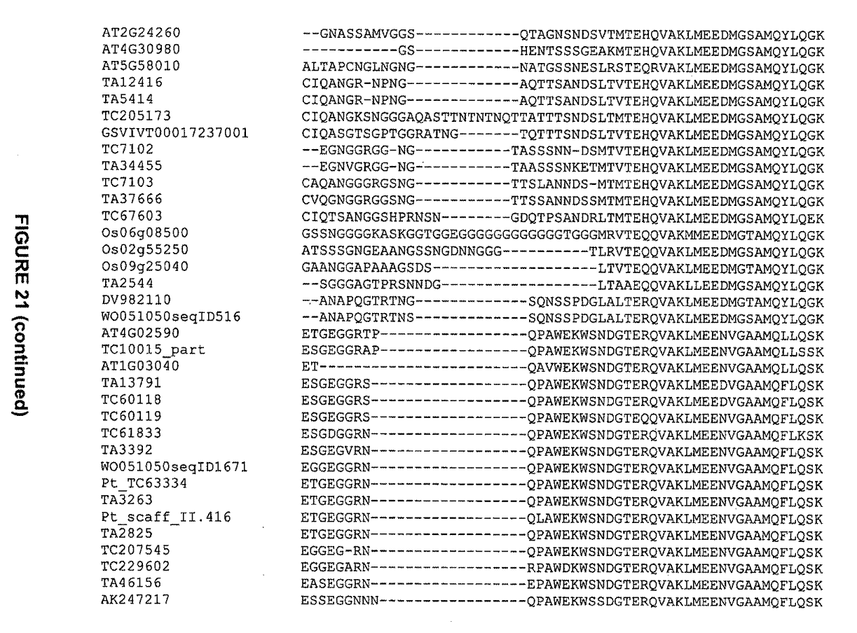

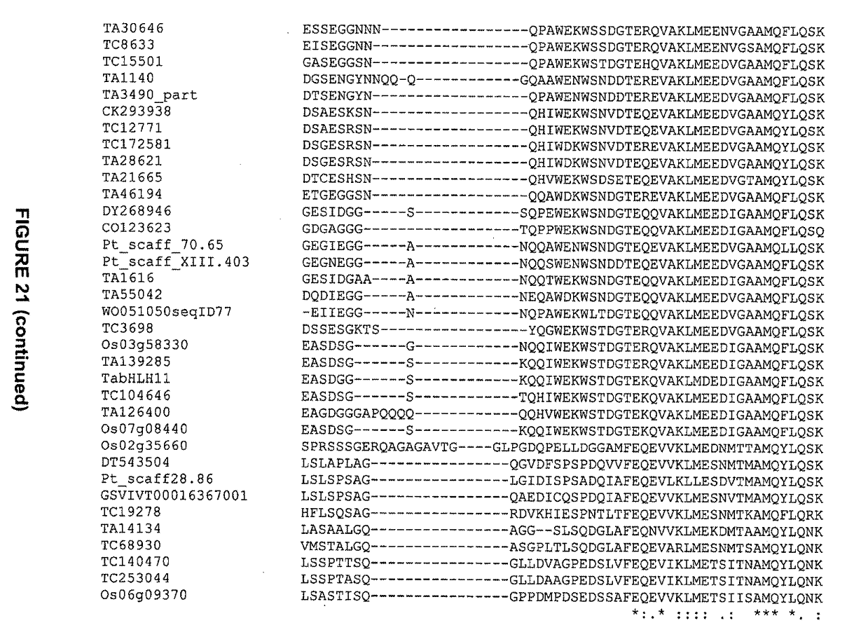

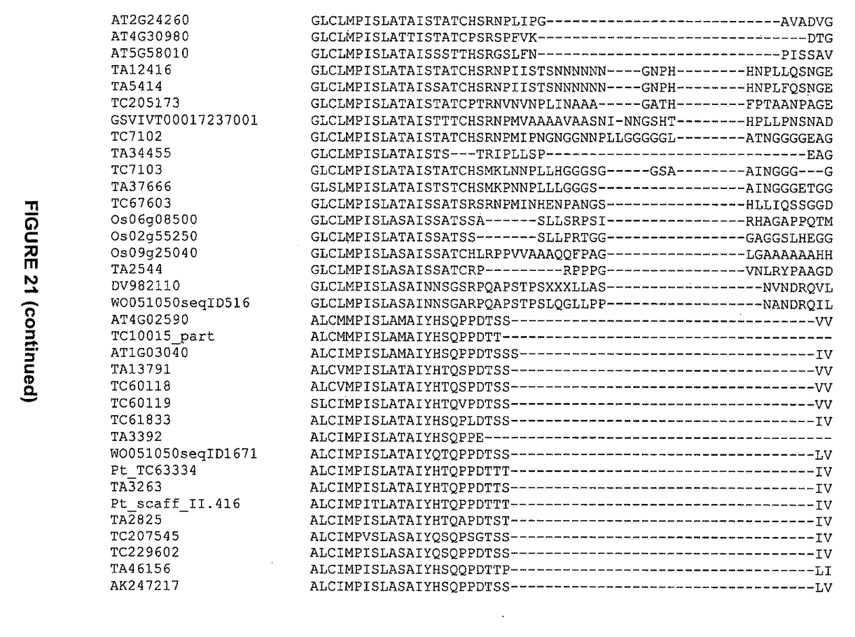

FIG. 20 represents the domain structure of SEQ ID NO: 245 with the conserved motifs indicated by underlining and their number. The HLH domain (motif 8) as determined by SMART is shown in bold underlined.

FIG. 21 represents a multiple alignment of various bHLH11-like proteins. A dot indicates conserved residues, a colon indicates highly conserved residues and an asterisk stands for perfectly conserved residues. The highest degree of sequence conservation is found in the region of the bHLH domain. The C-terminal part of AT2G24260 that extends beyond the other proteins in the alignment is deleted.

FIG. 22 Circular cladogram of selected bHLH proteins. bHLH11-like proteins and one Arabidopsis protein representing each of the other classes defined by Heim 2003 were used. The alignment was generated using “CLUSTALX”, and a neighbour-joining tree was calculated. The circular cladogram was drawn using Dendroscope (Huson et al. BMC Bioinformatics 2007). Bootstrap results for 100 replicates is indicated for some major nodes; the boxed bootstrap value shows that the group of bHLH11-like proteins is clearly delineated from the other bHLH proteins.

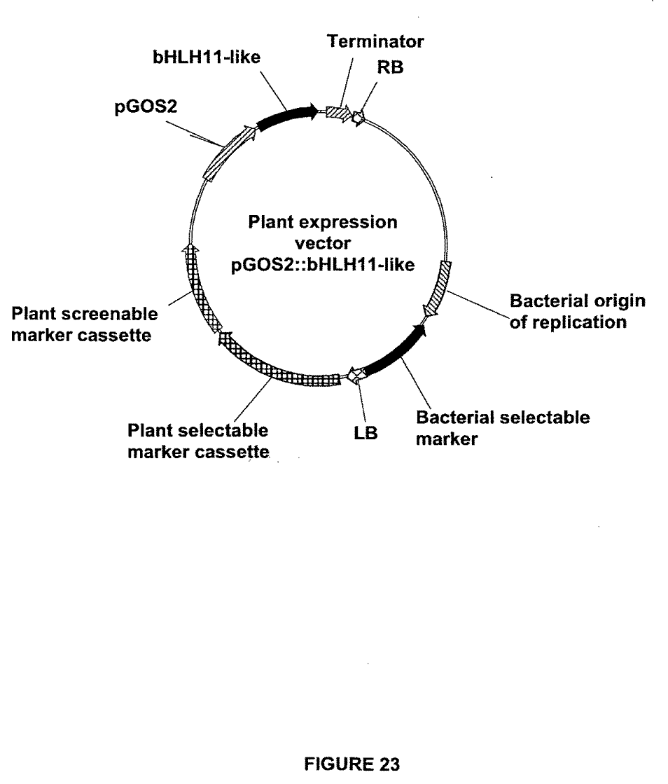

FIG. 23 represents the binary vector for increased expression in Oryza sativa of a bHLH11-like-encoding nucleic acid under the control of a rice GOS2 promoter (pGOS2).

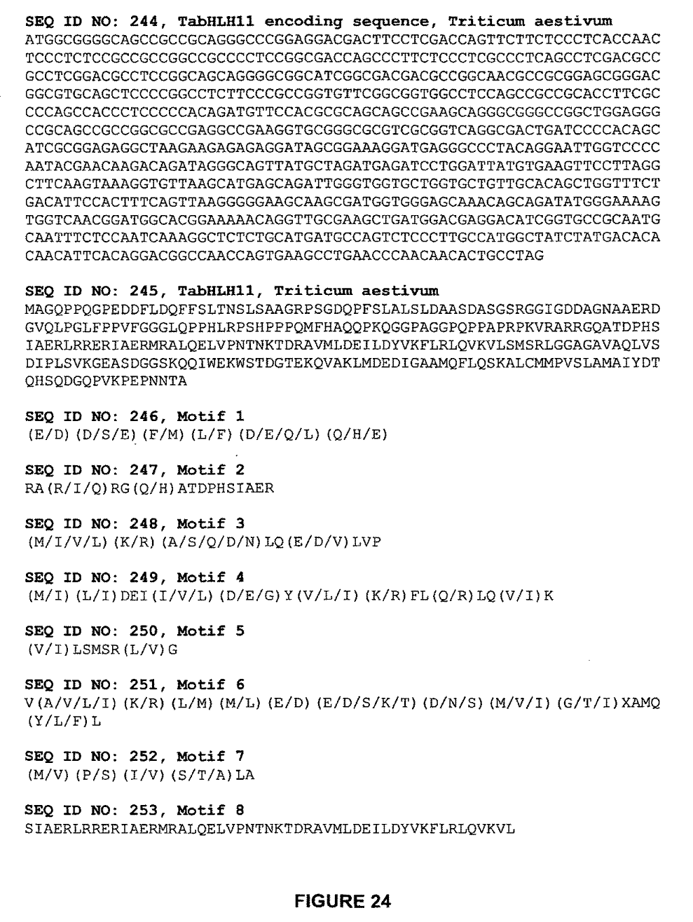

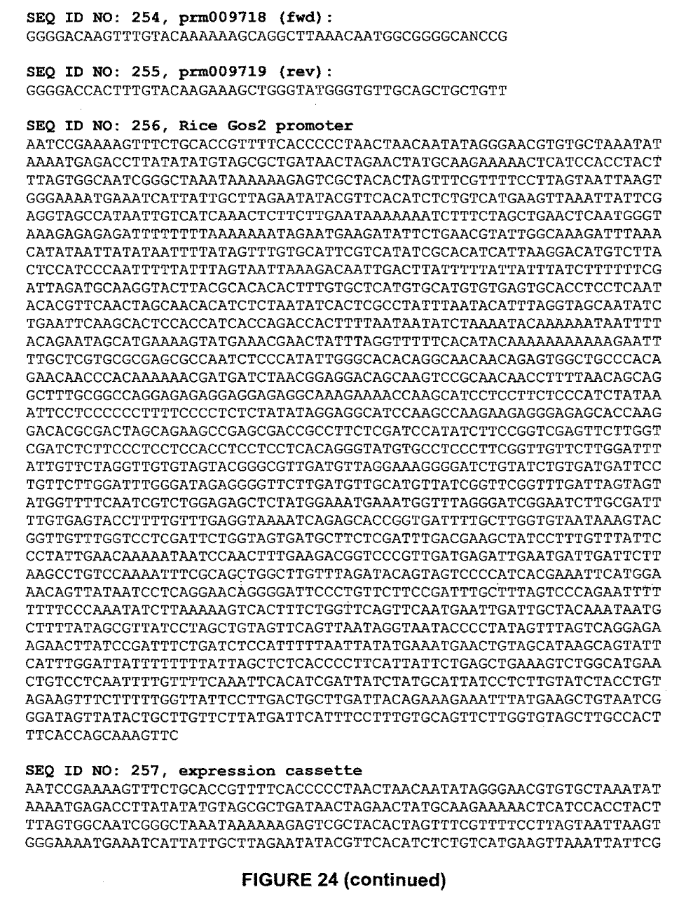

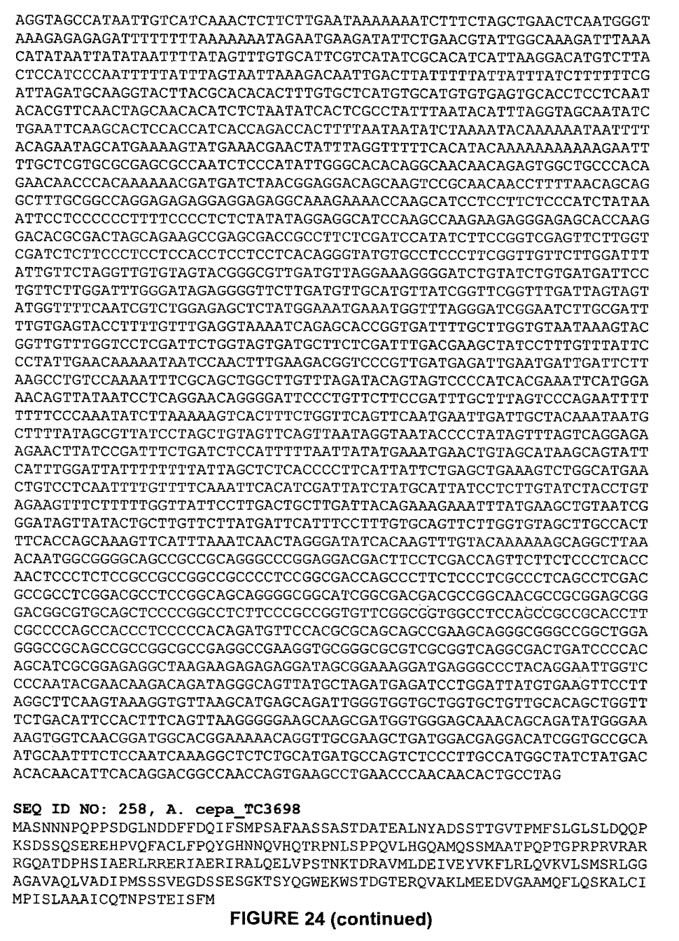

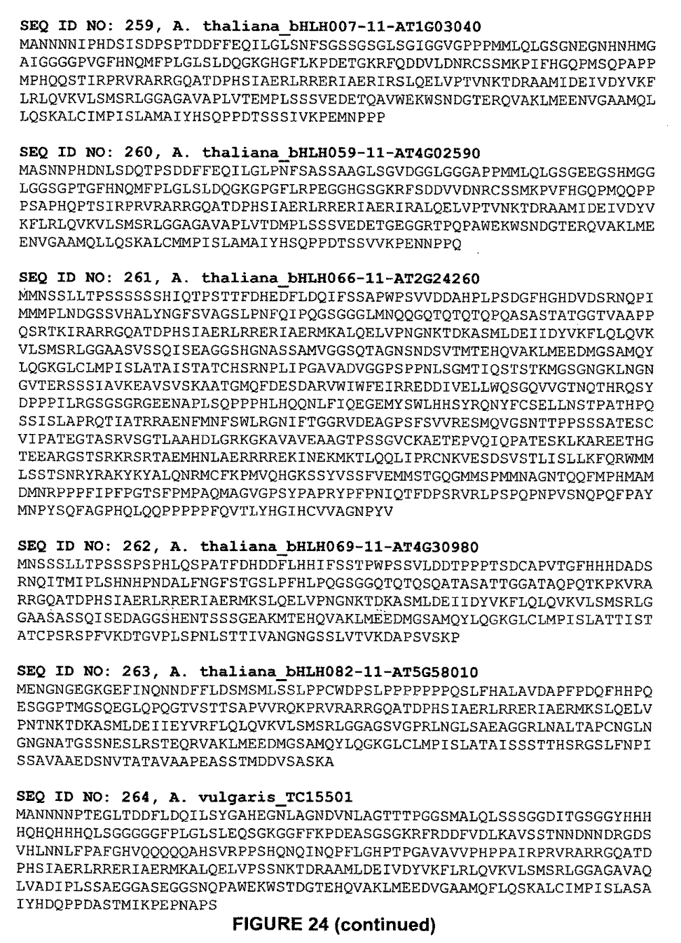

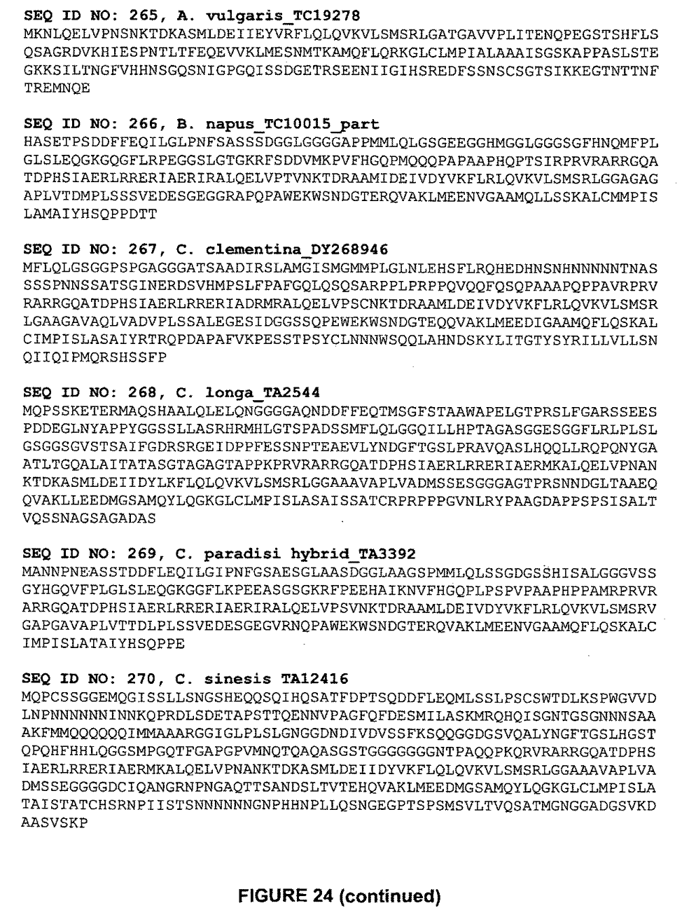

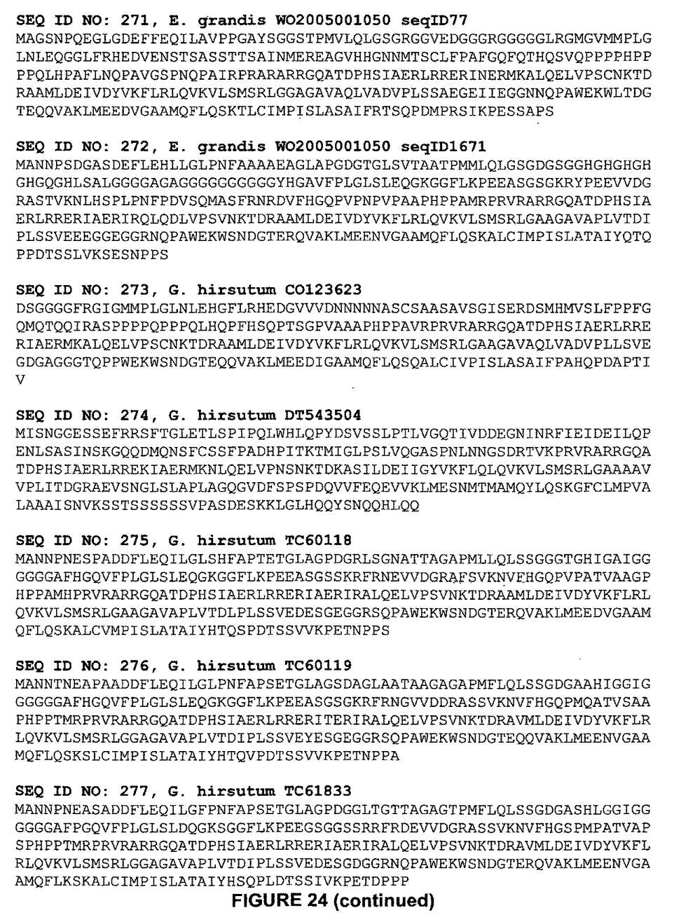

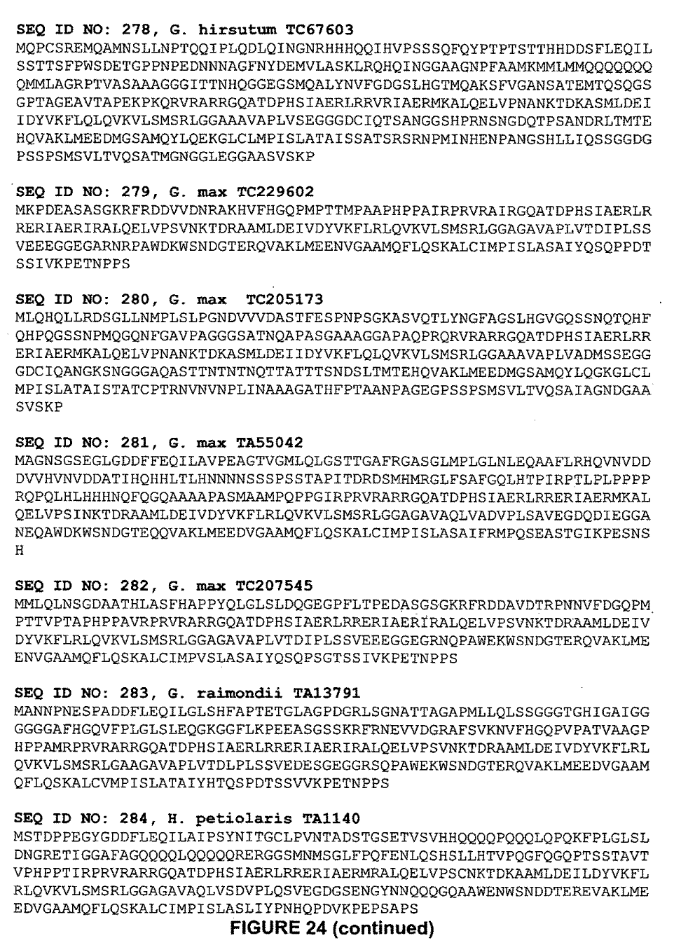

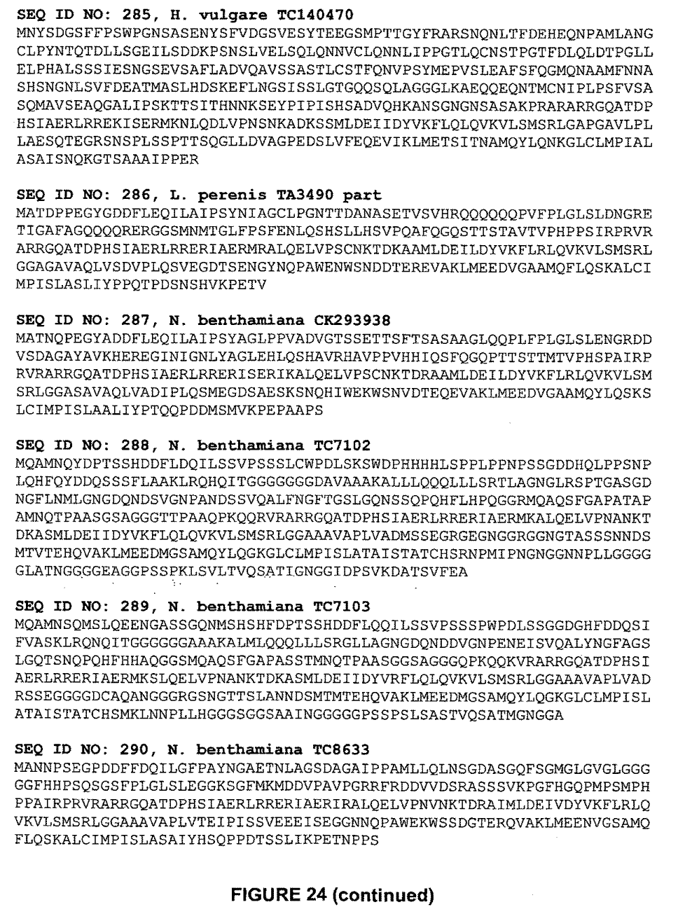

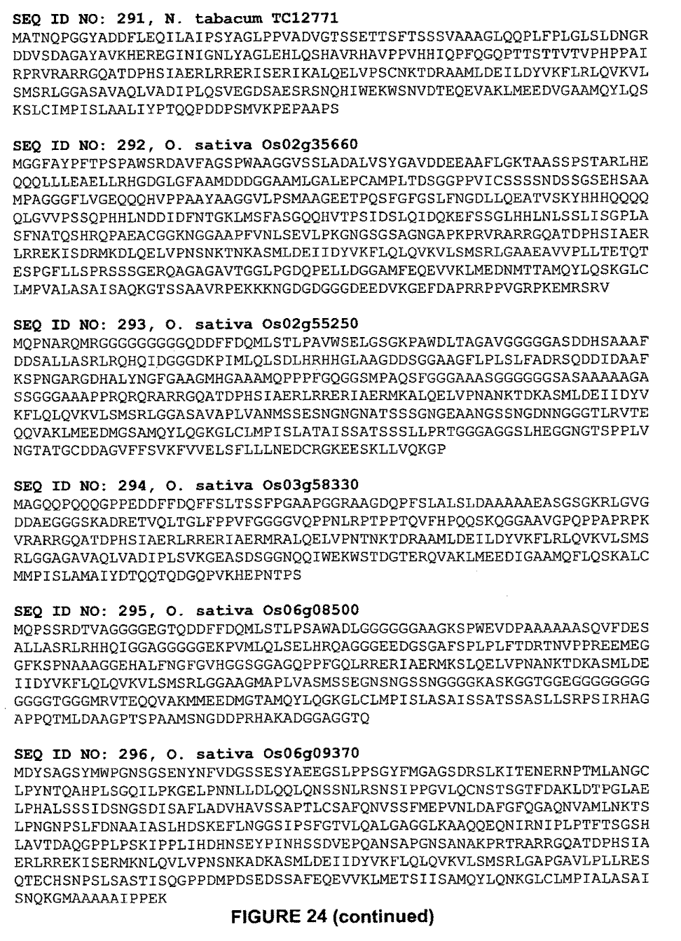

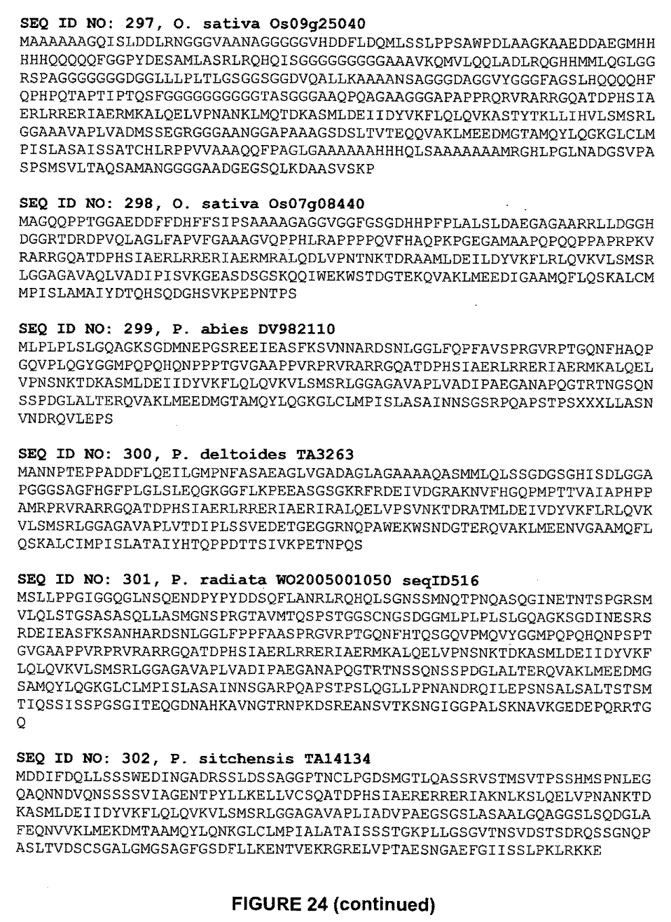

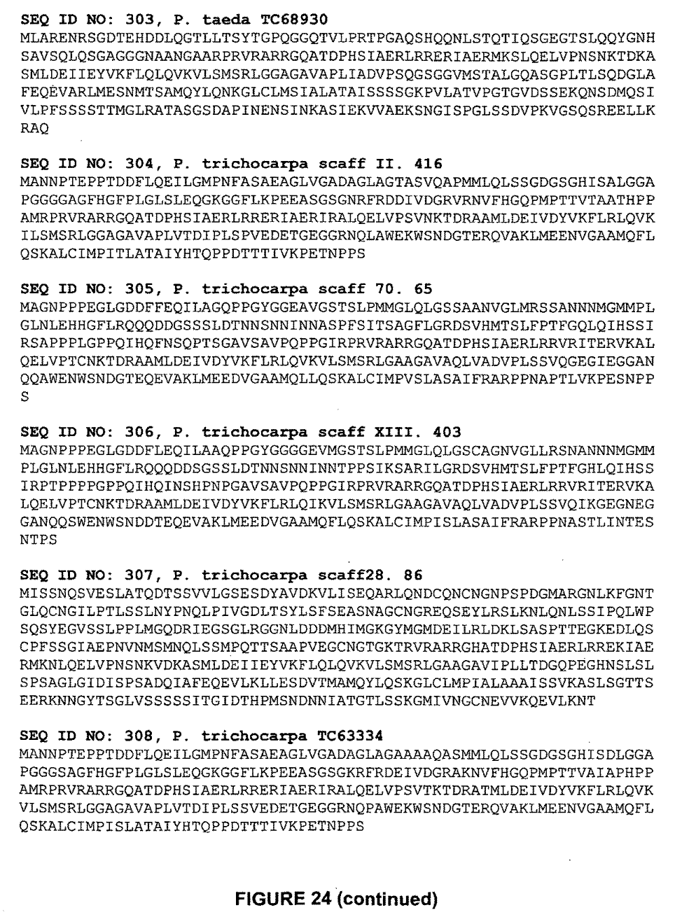

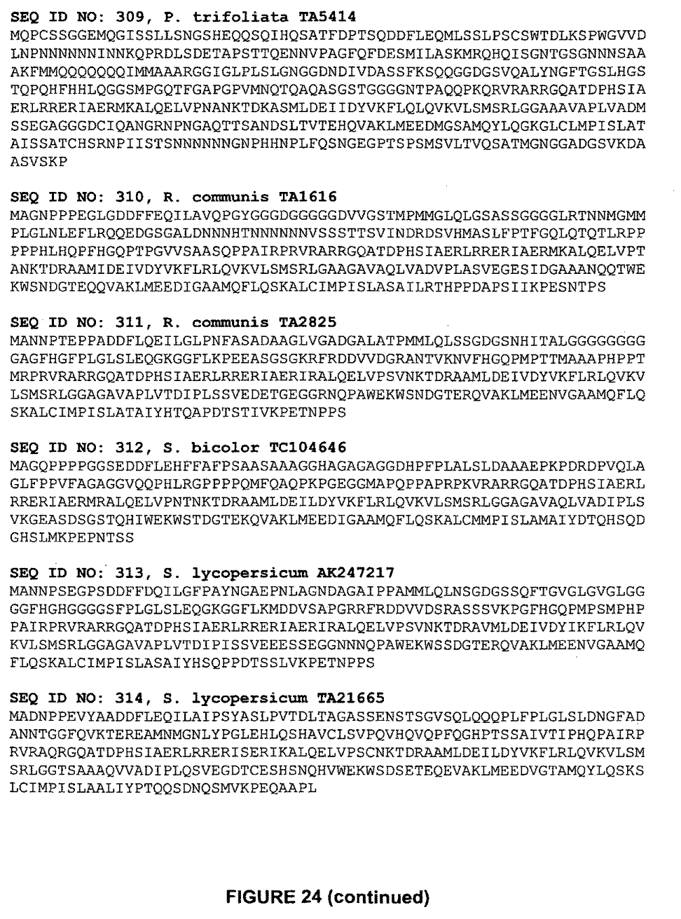

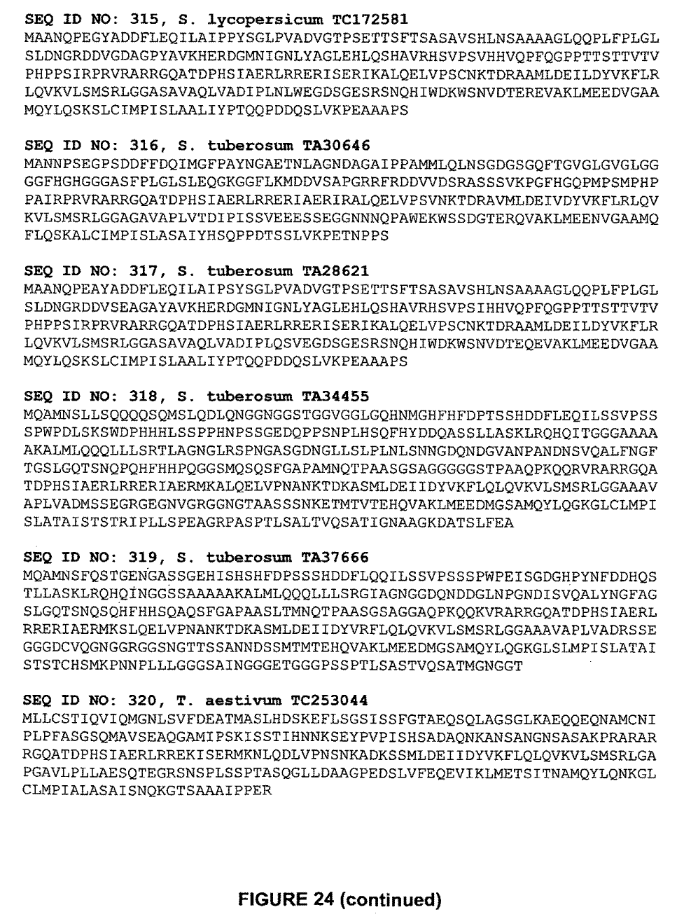

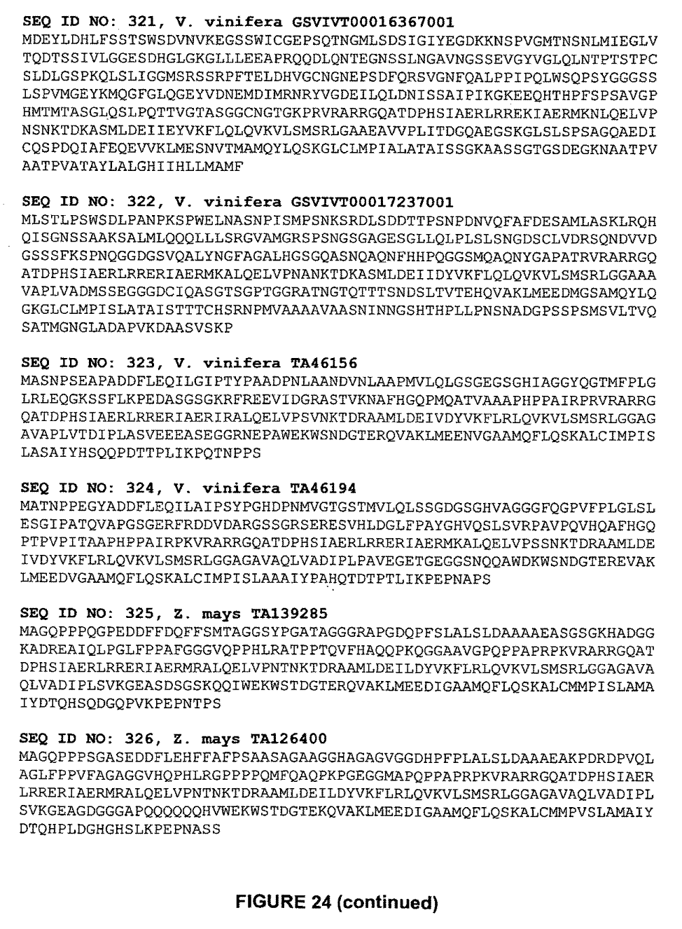

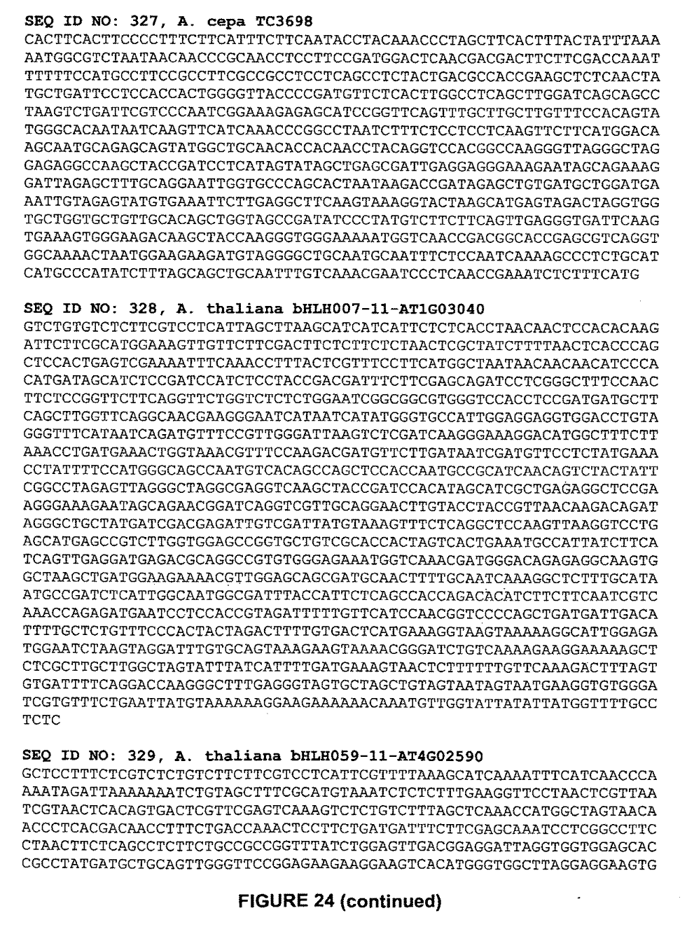

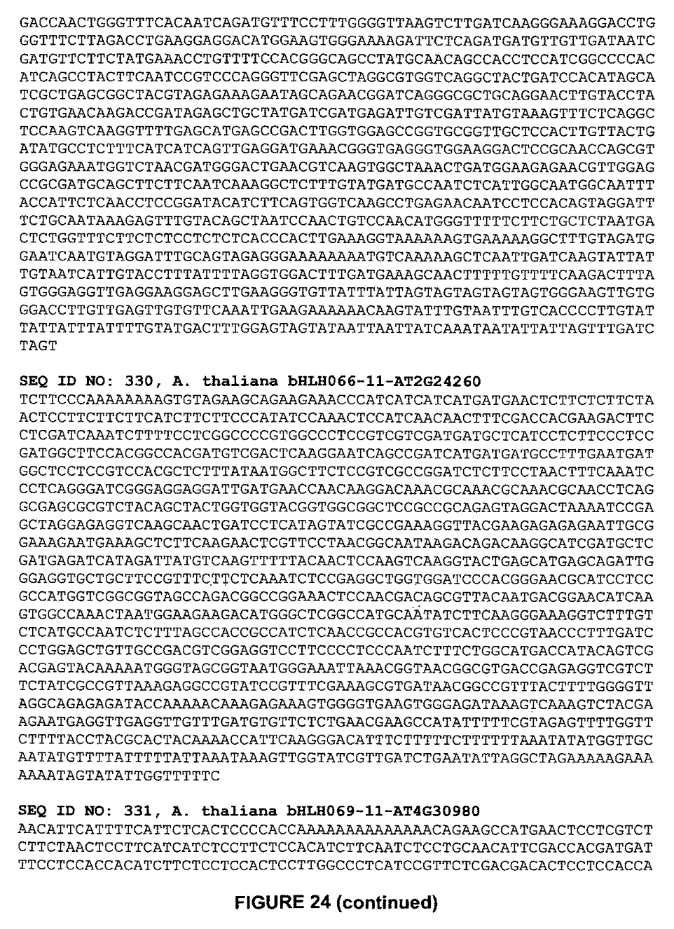

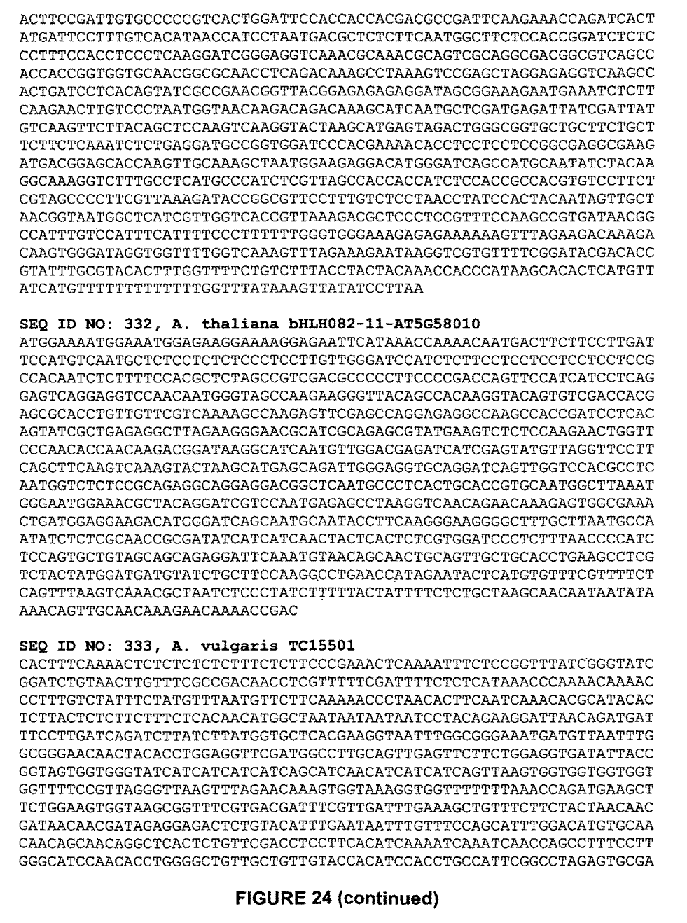

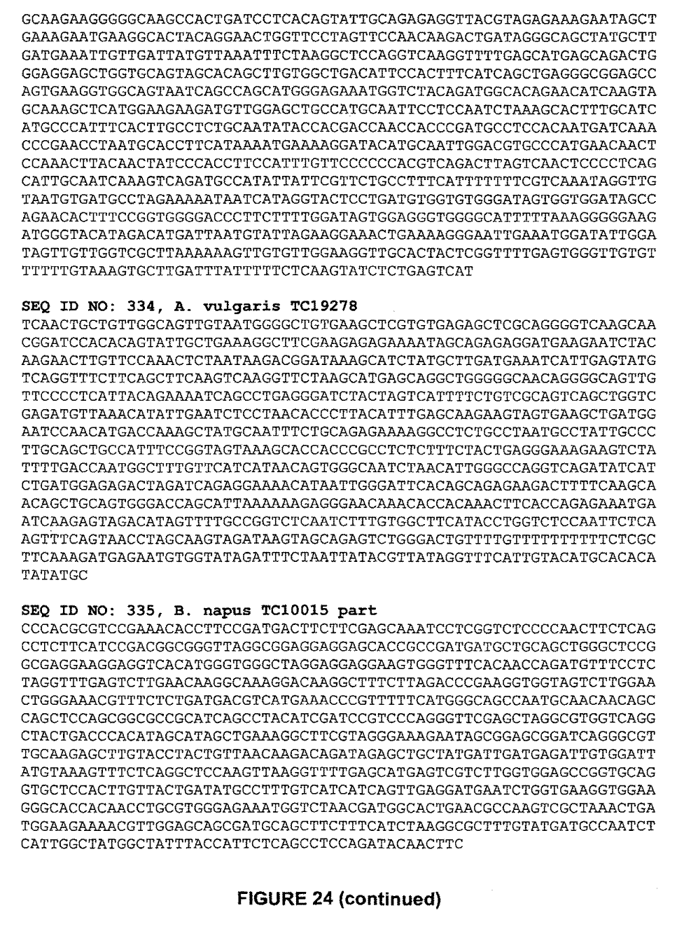

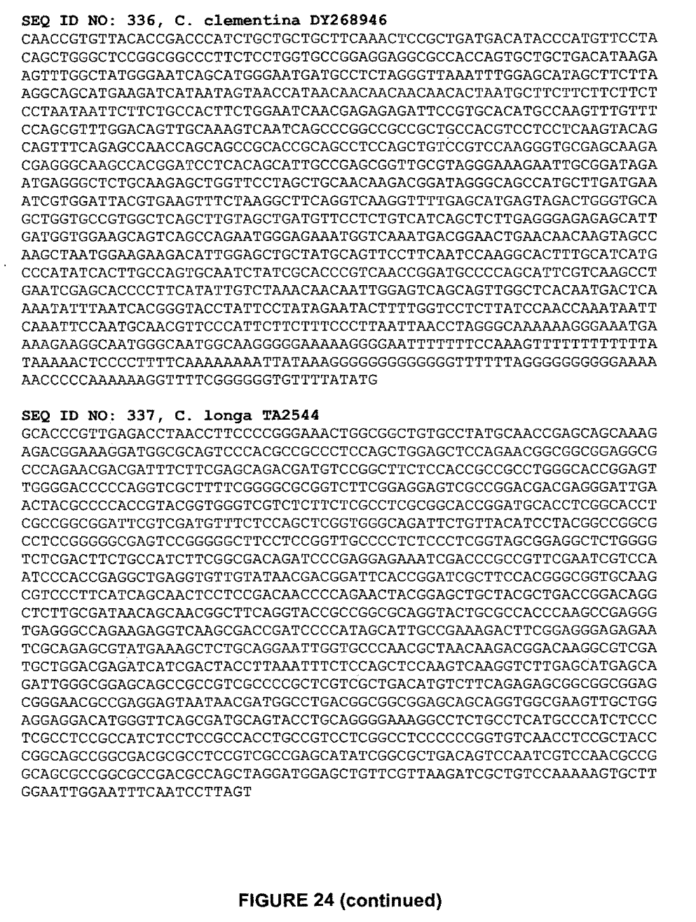

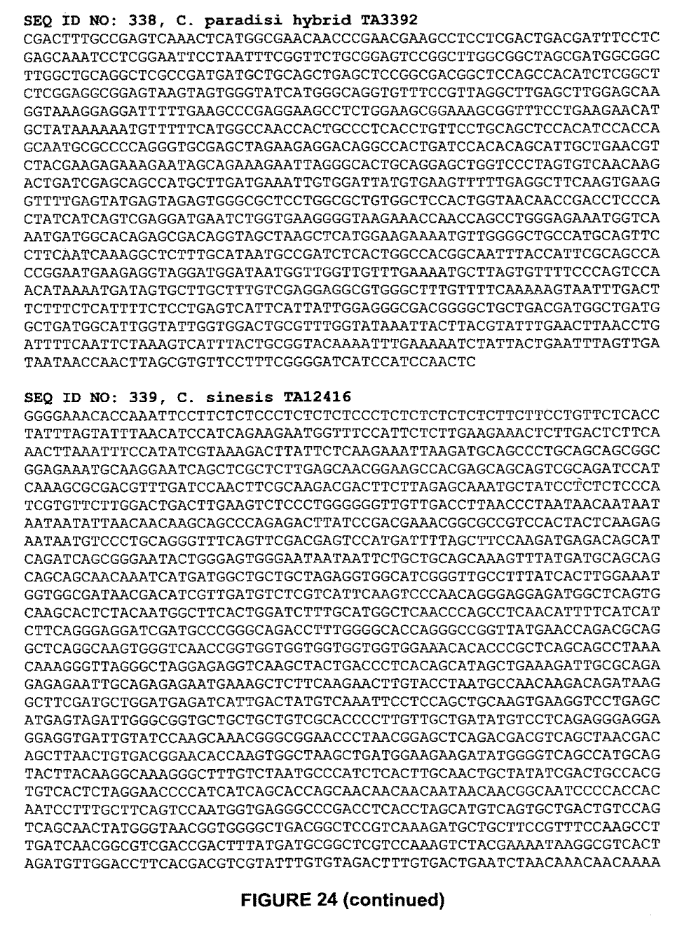

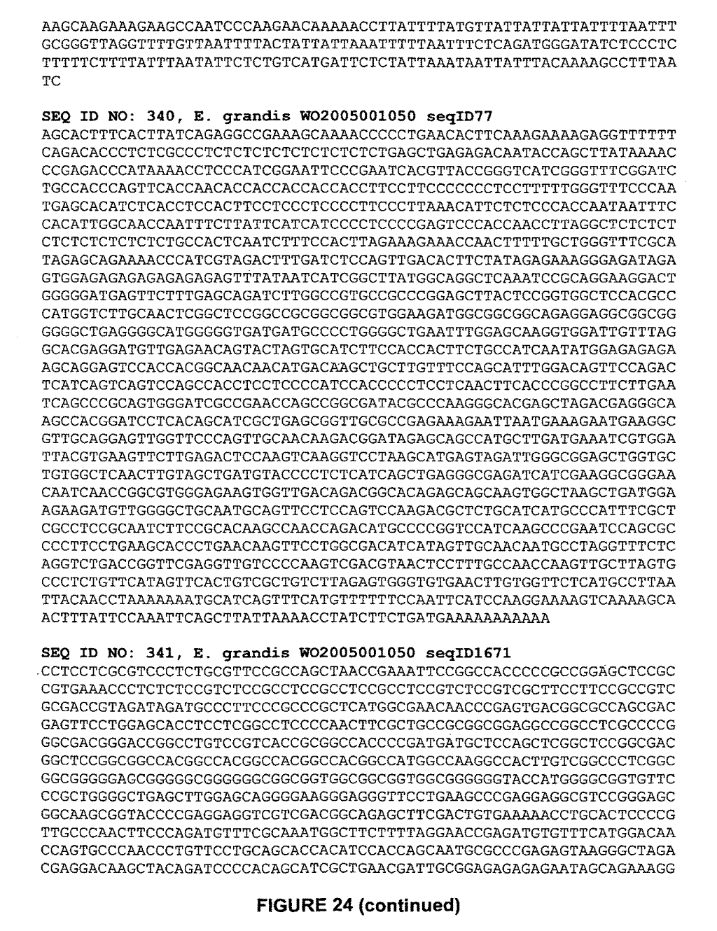

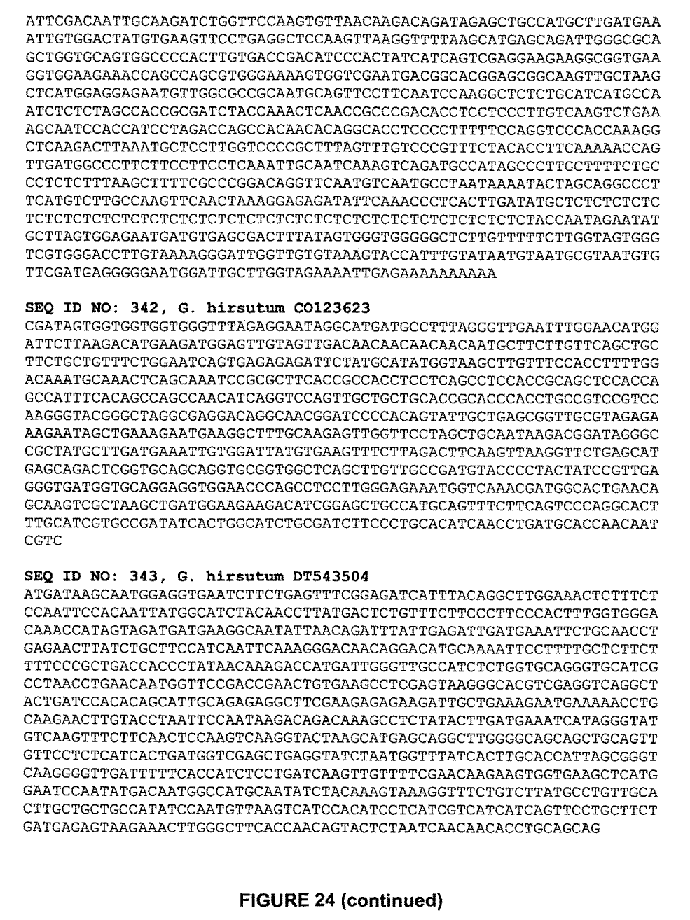

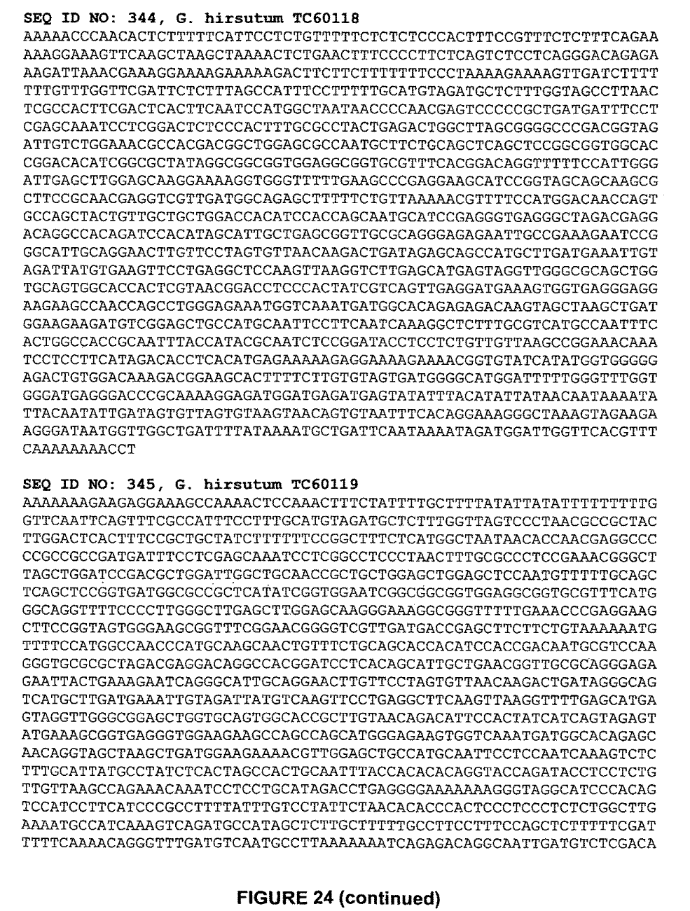

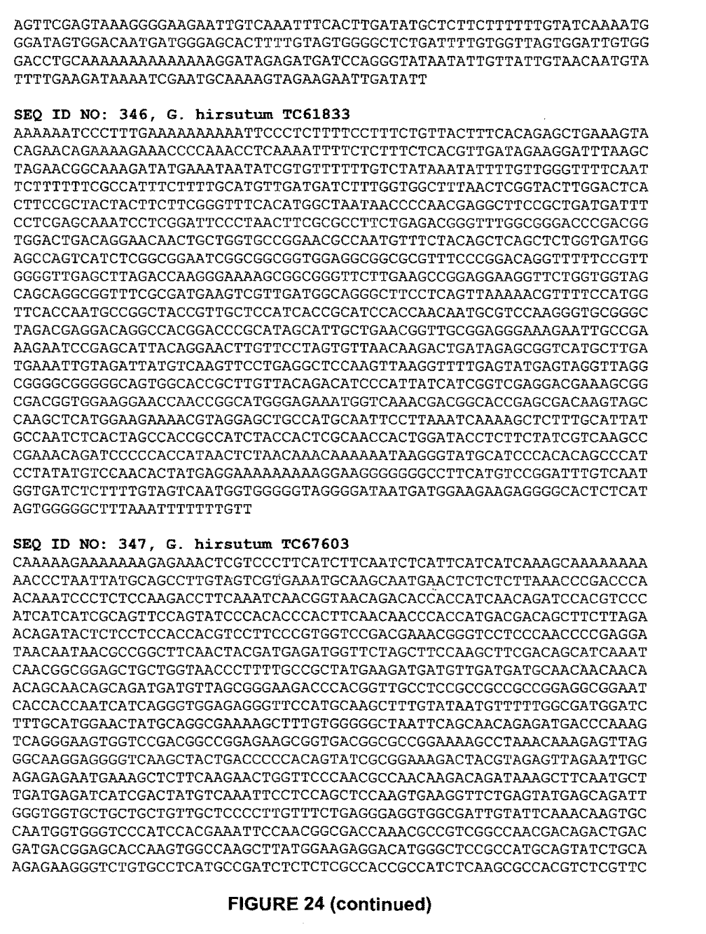

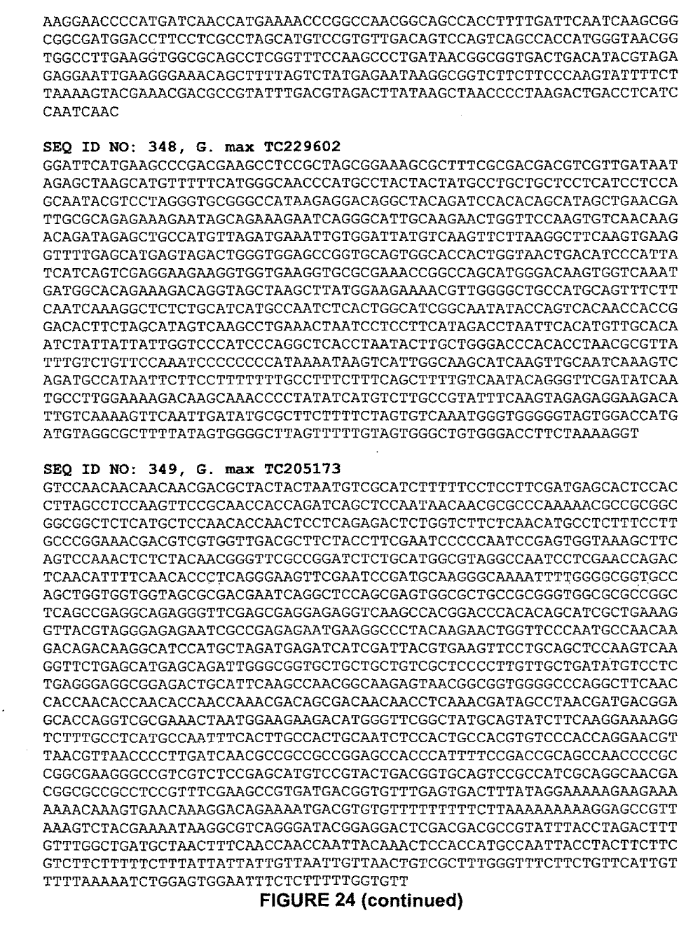

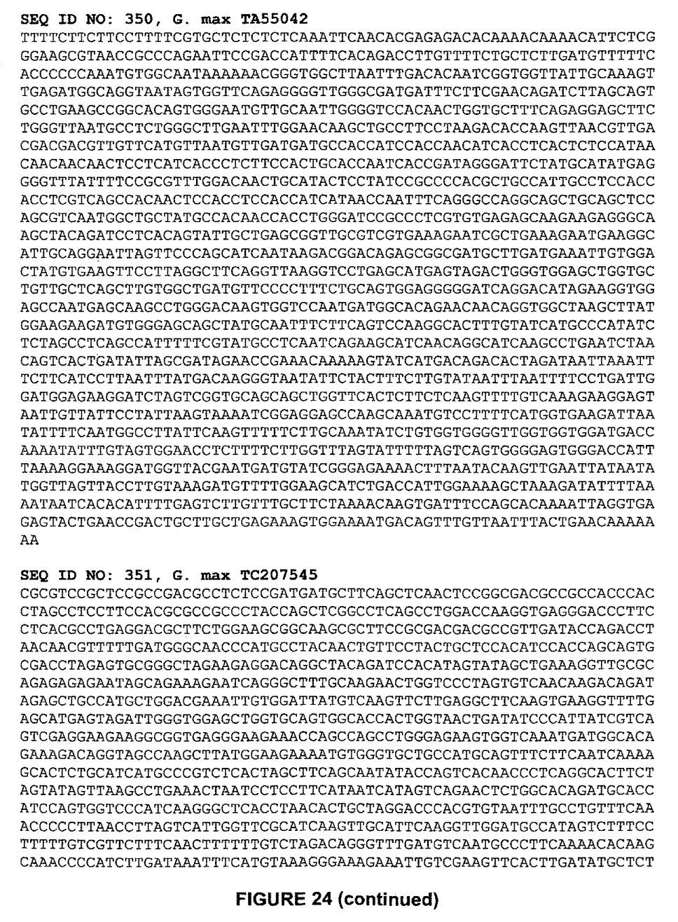

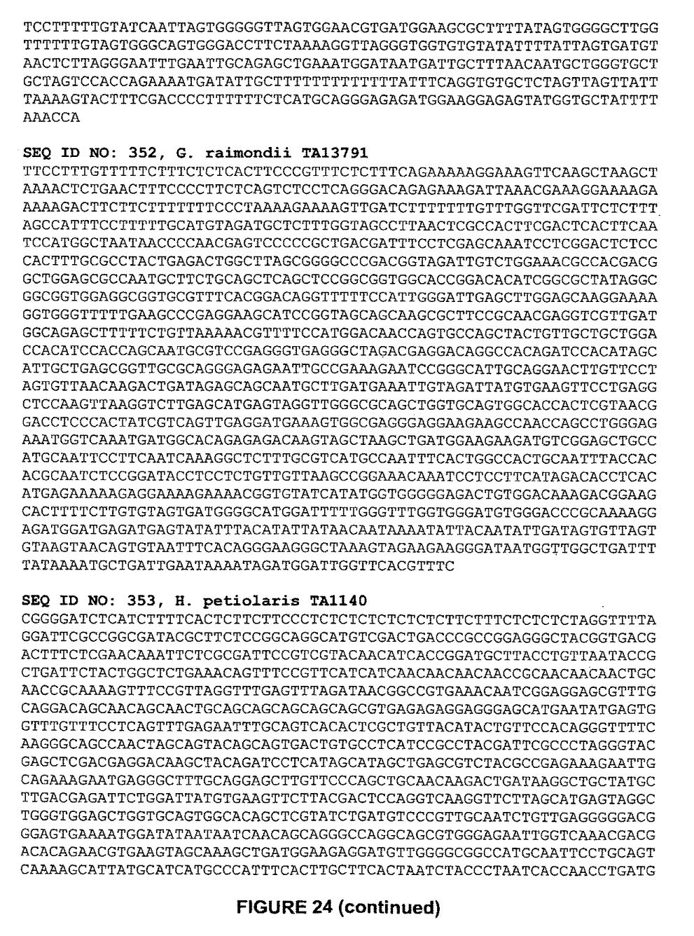

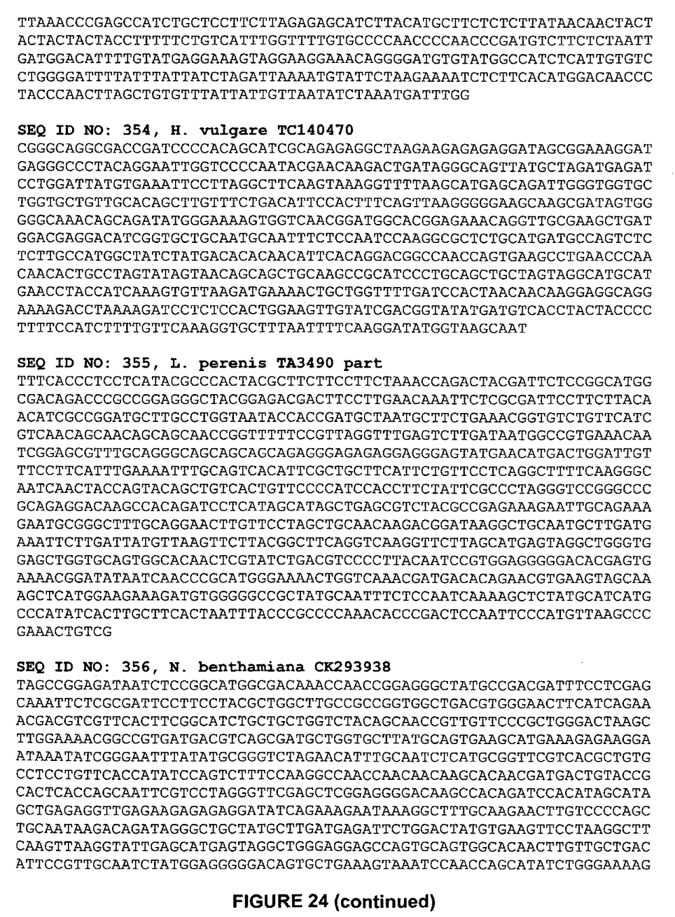

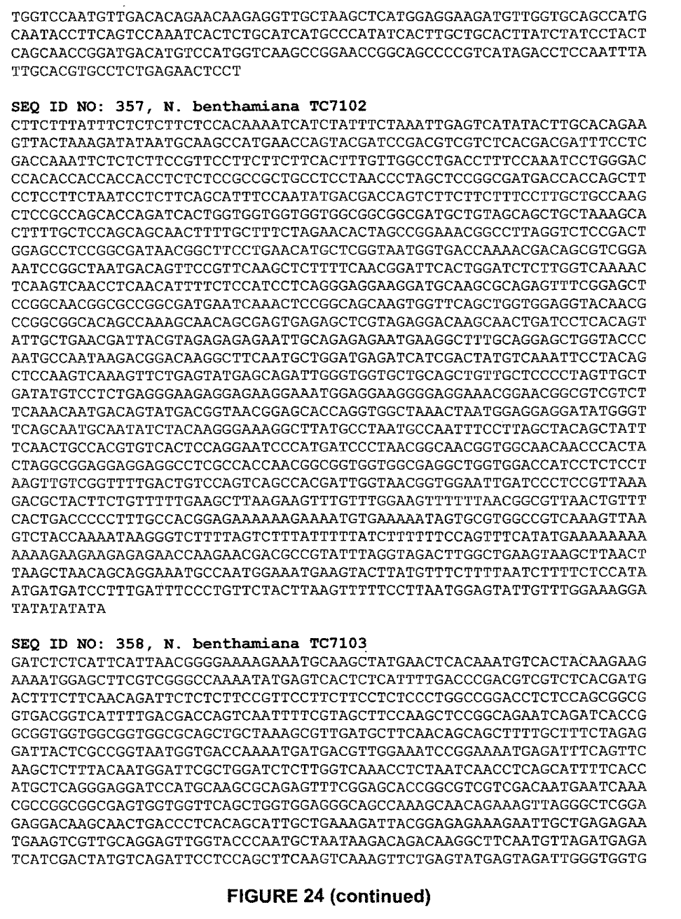

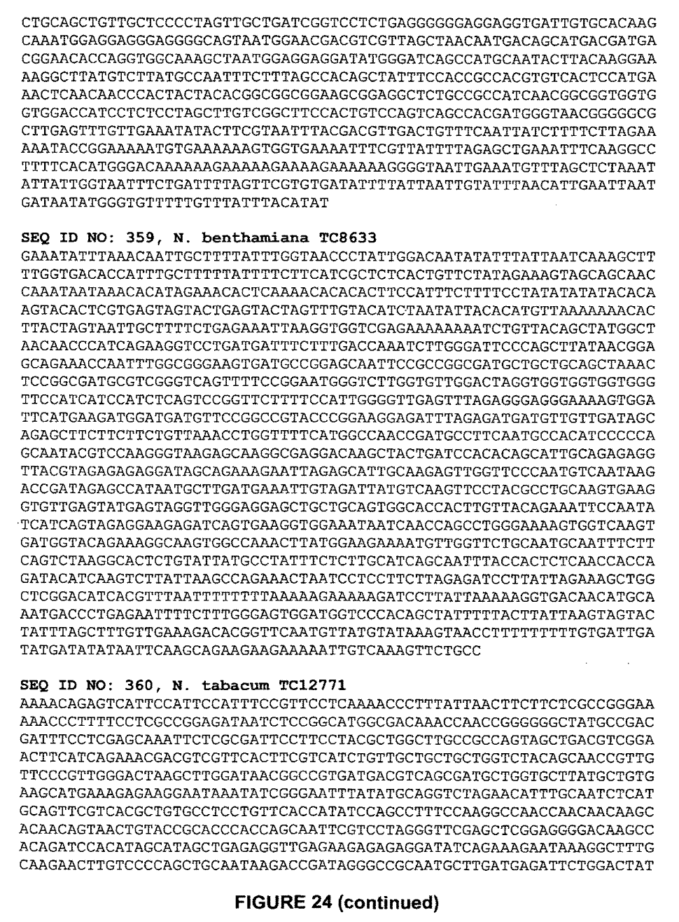

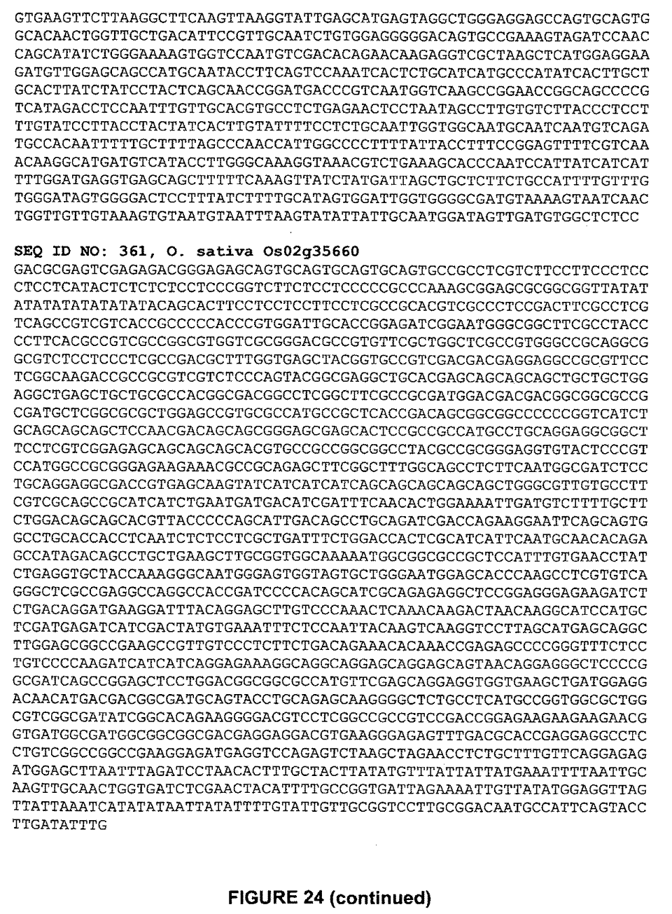

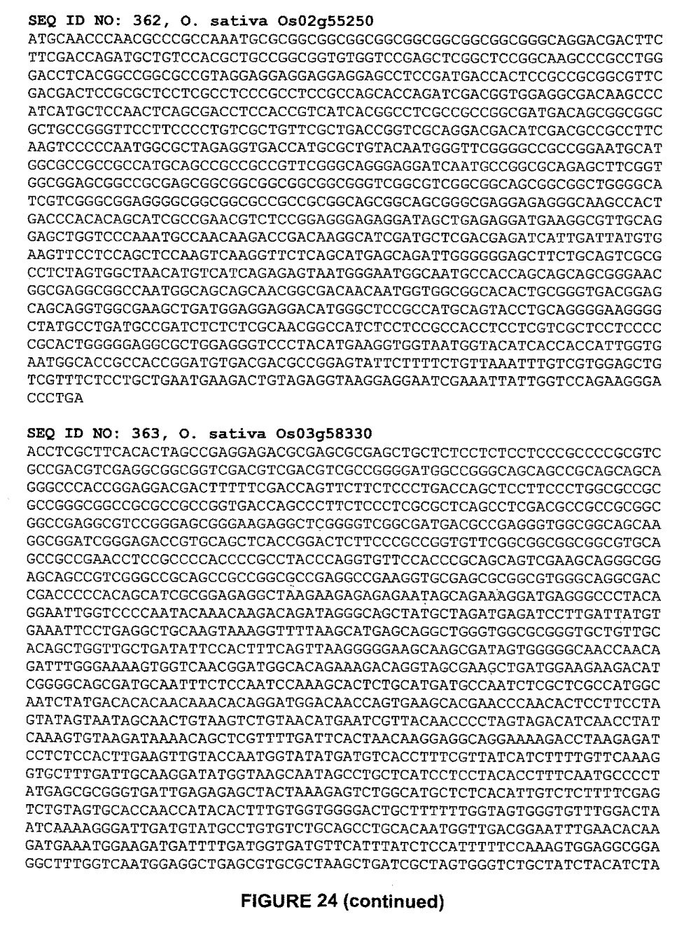

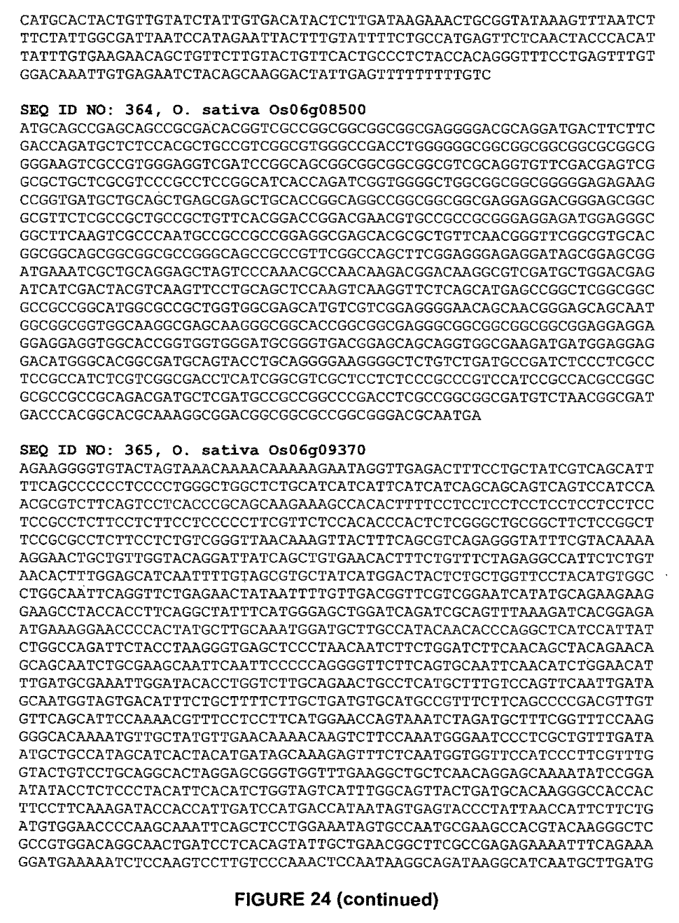

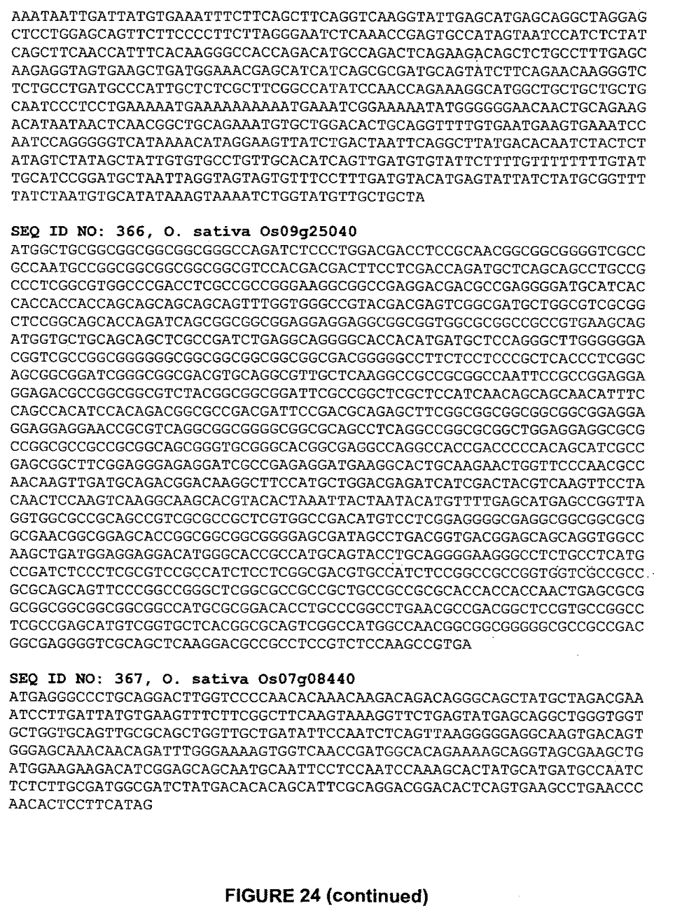

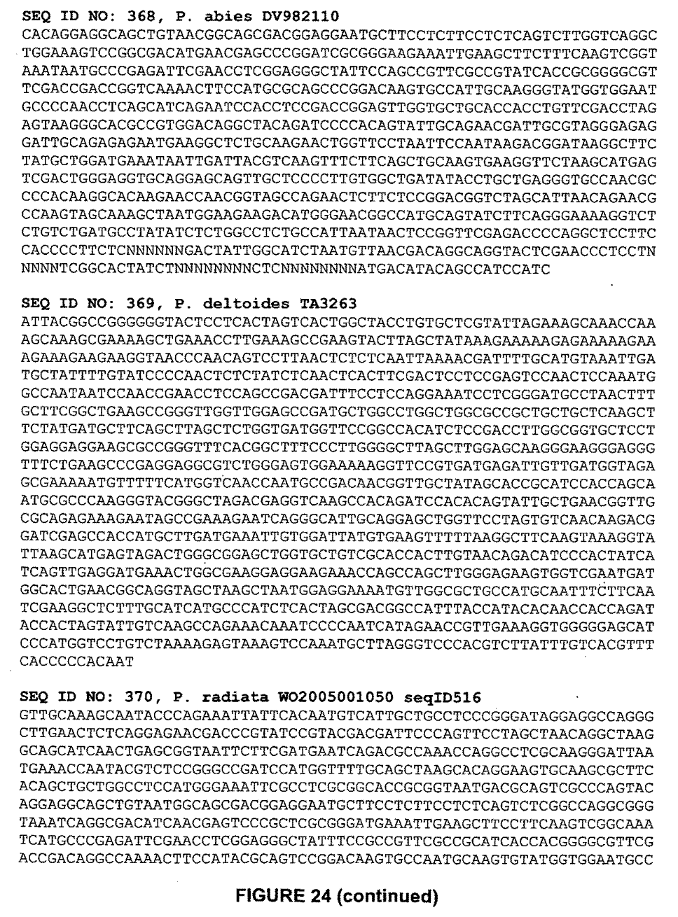

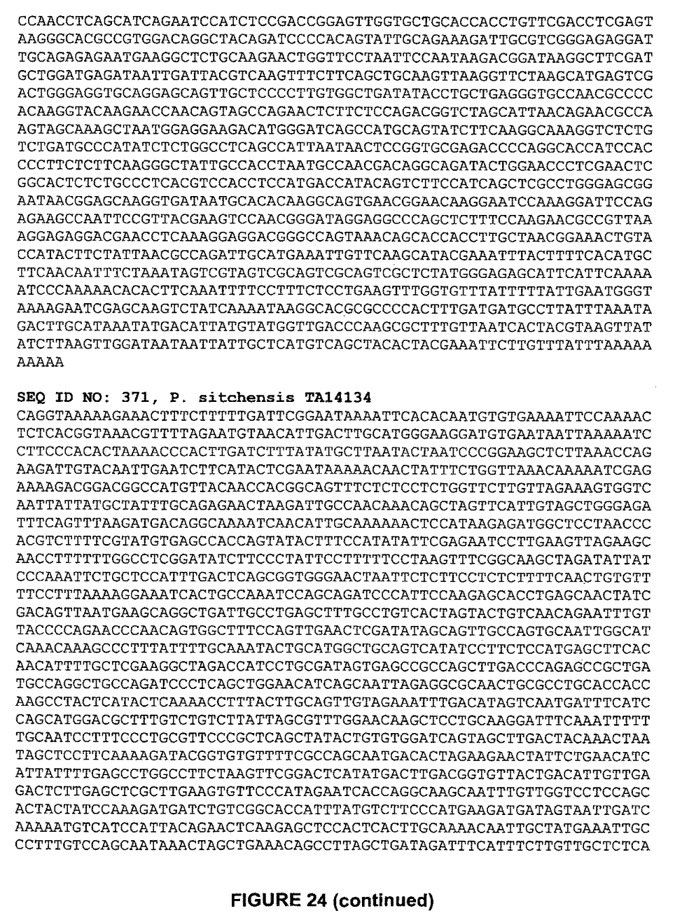

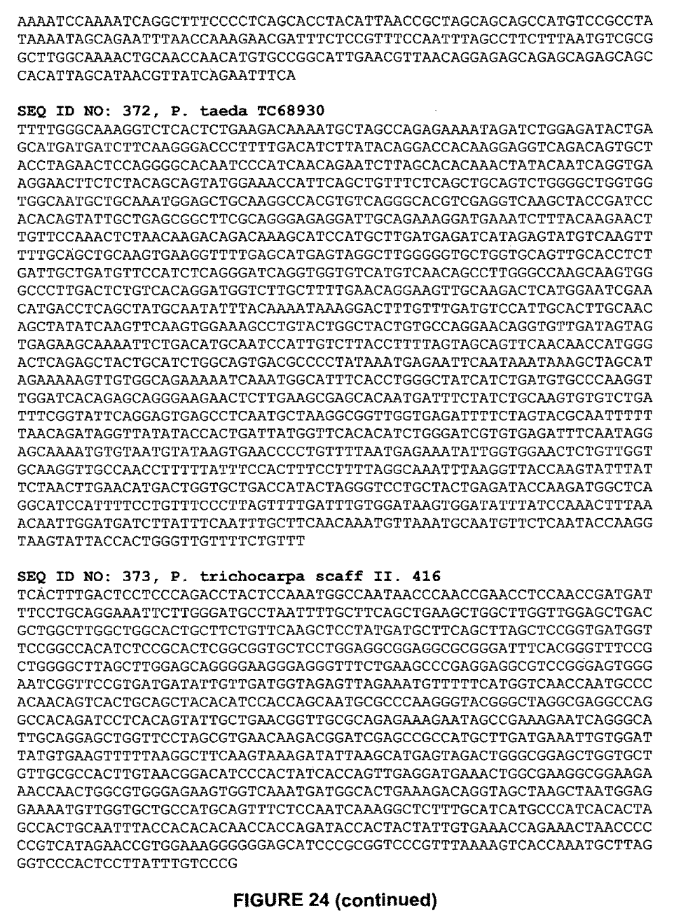

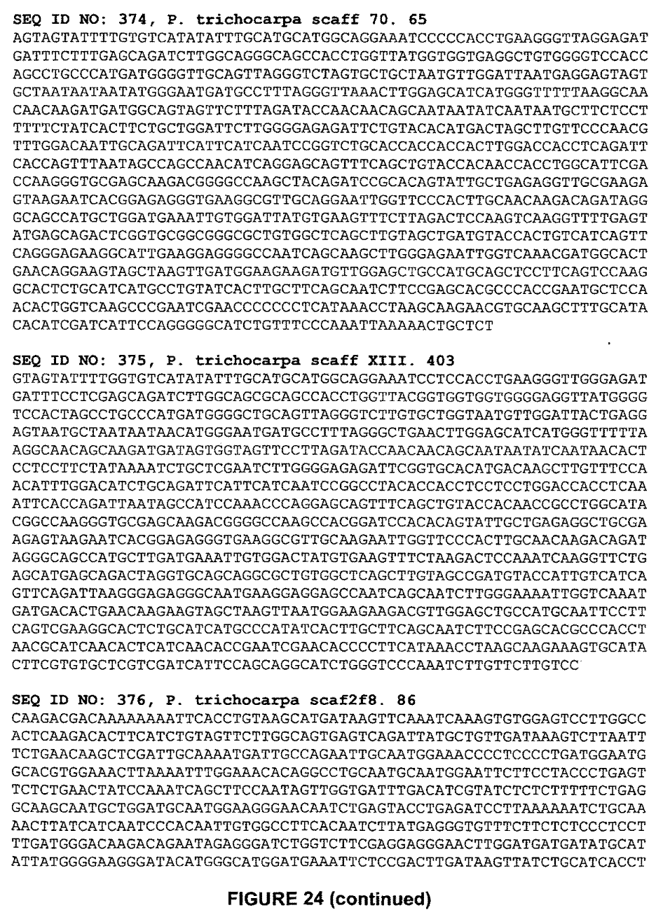

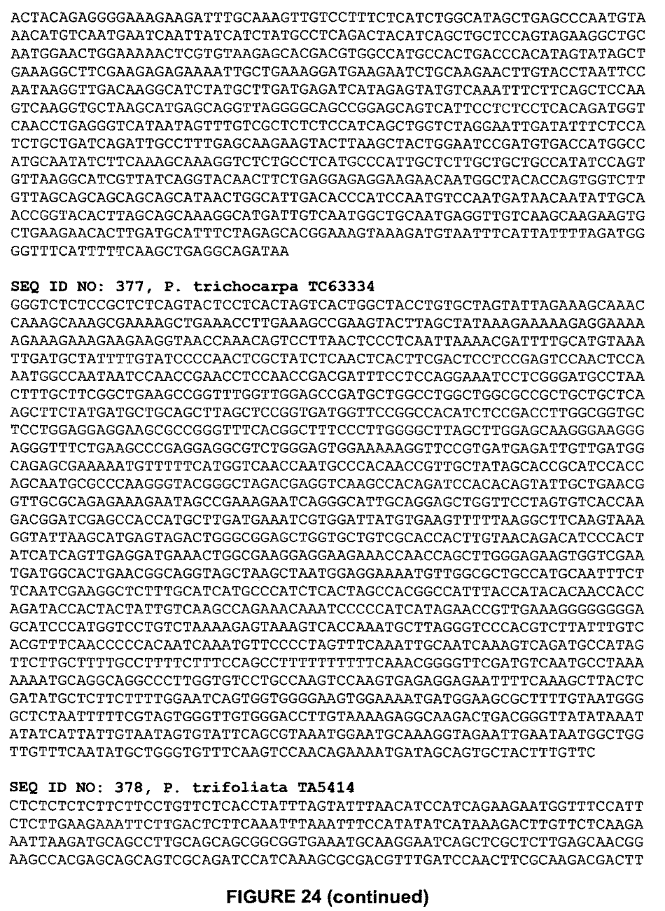

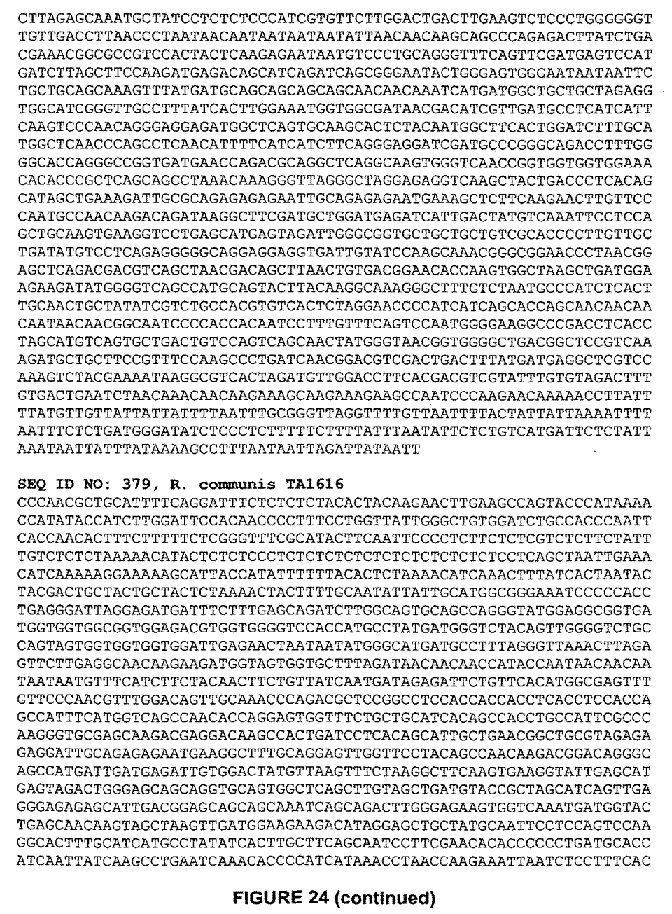

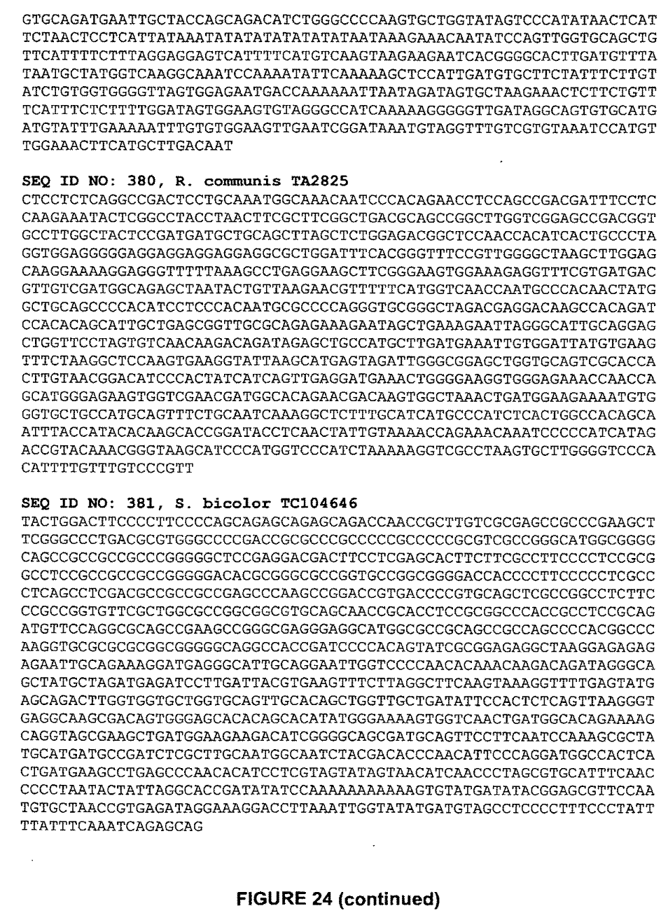

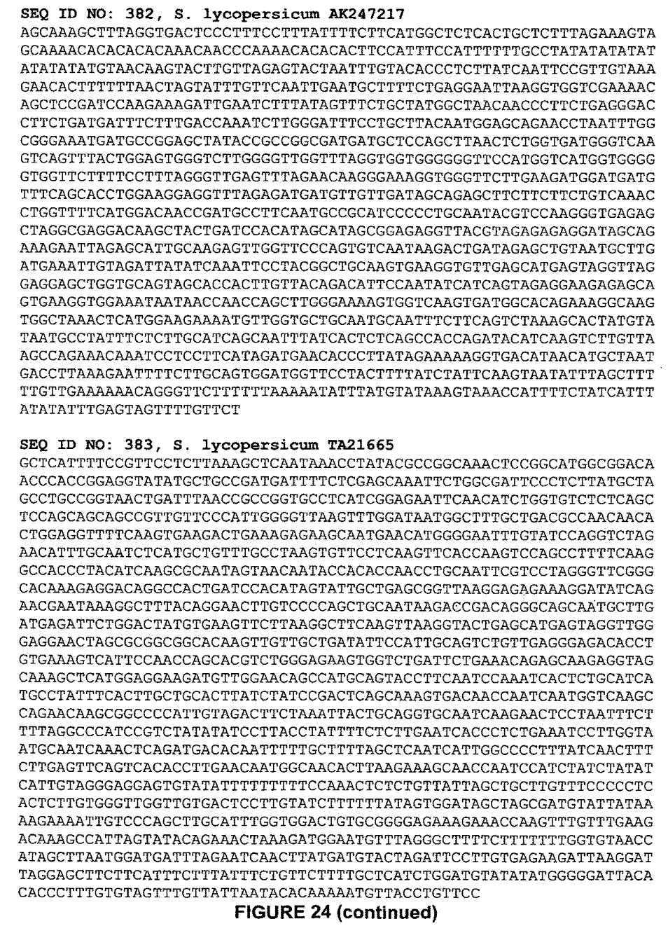

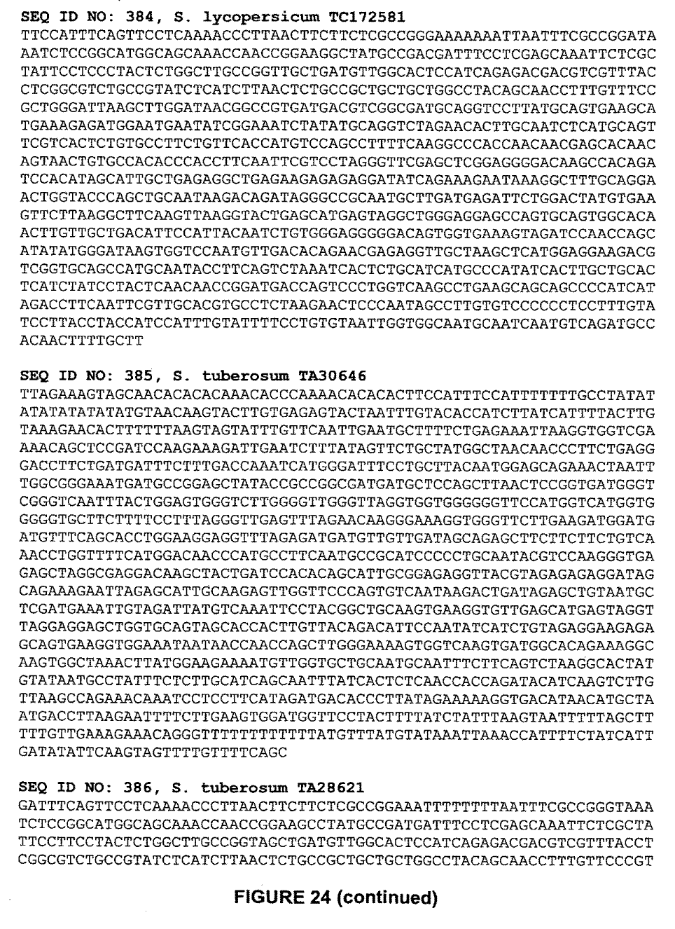

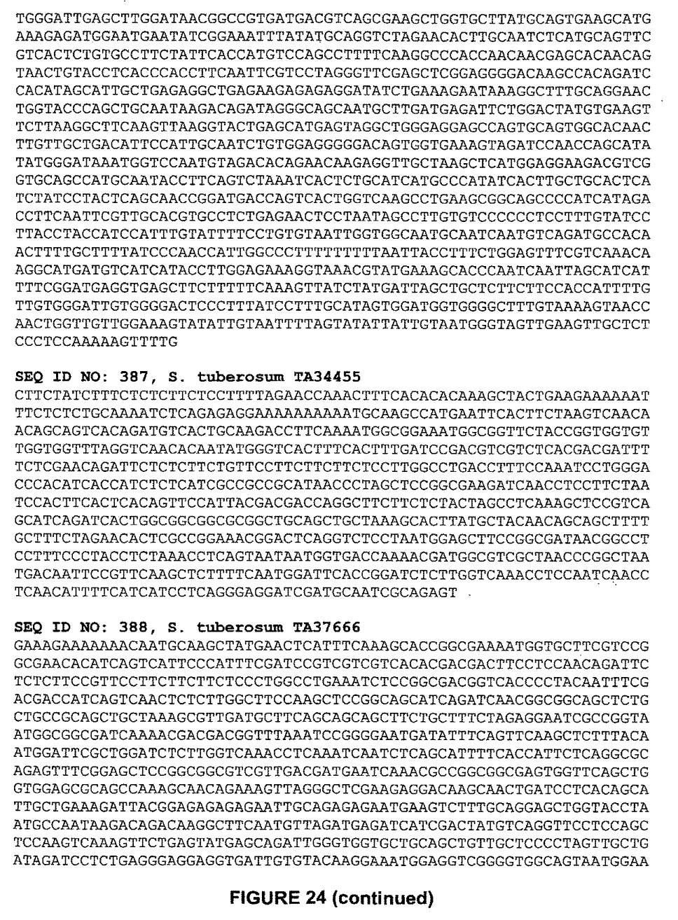

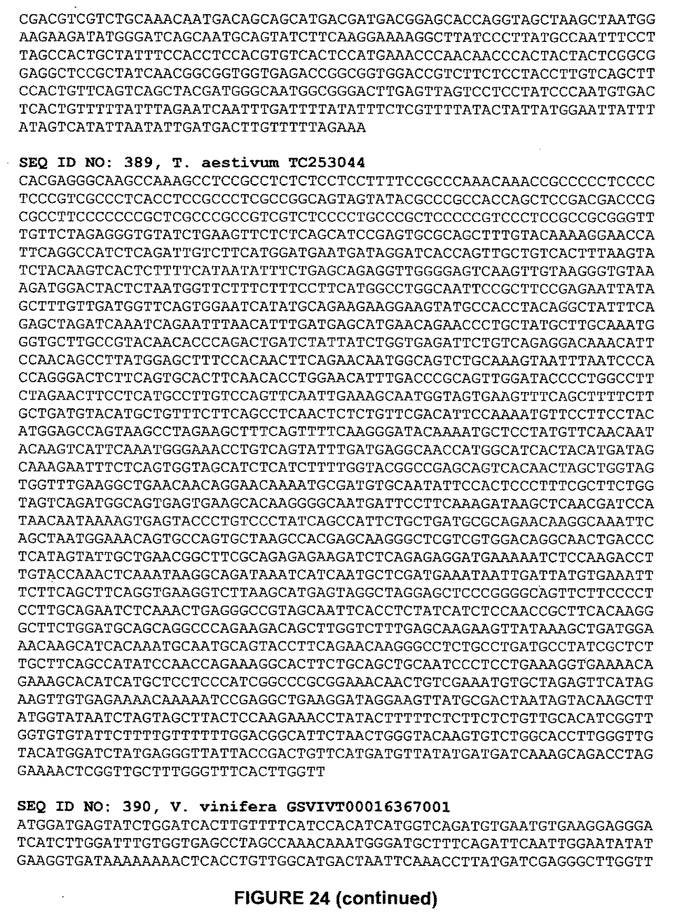

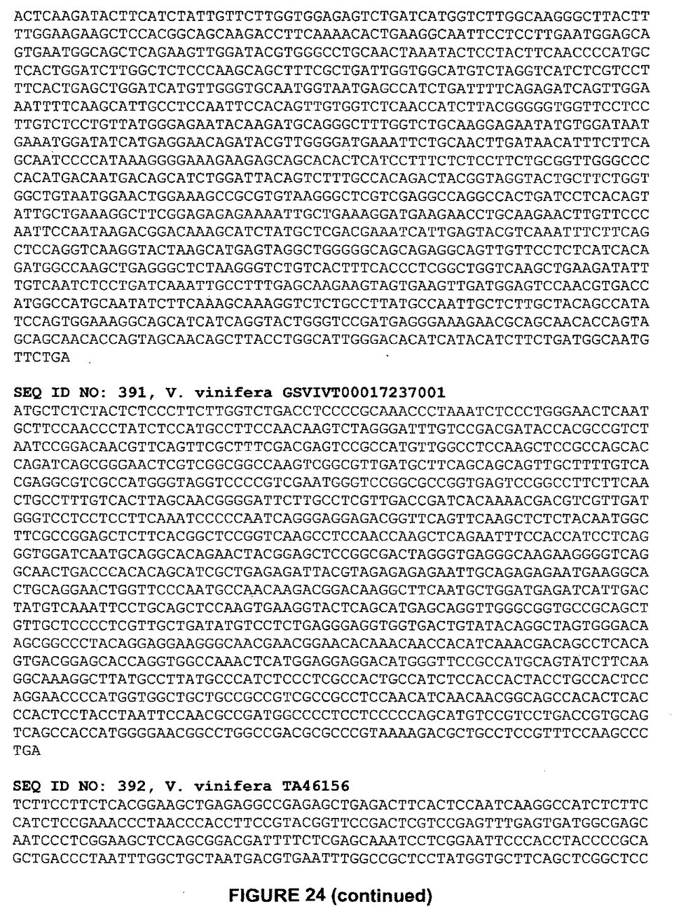

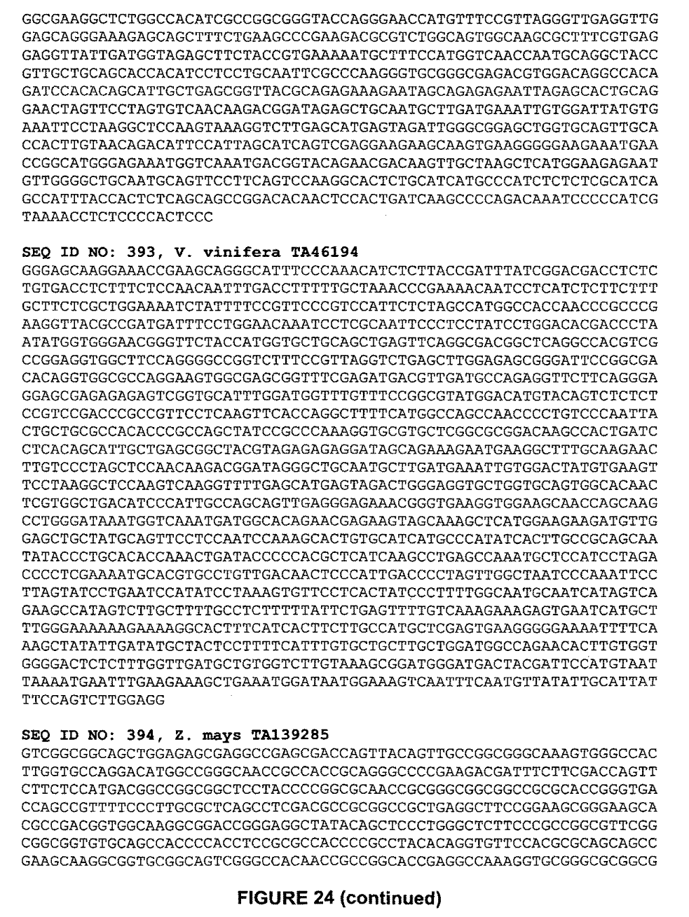

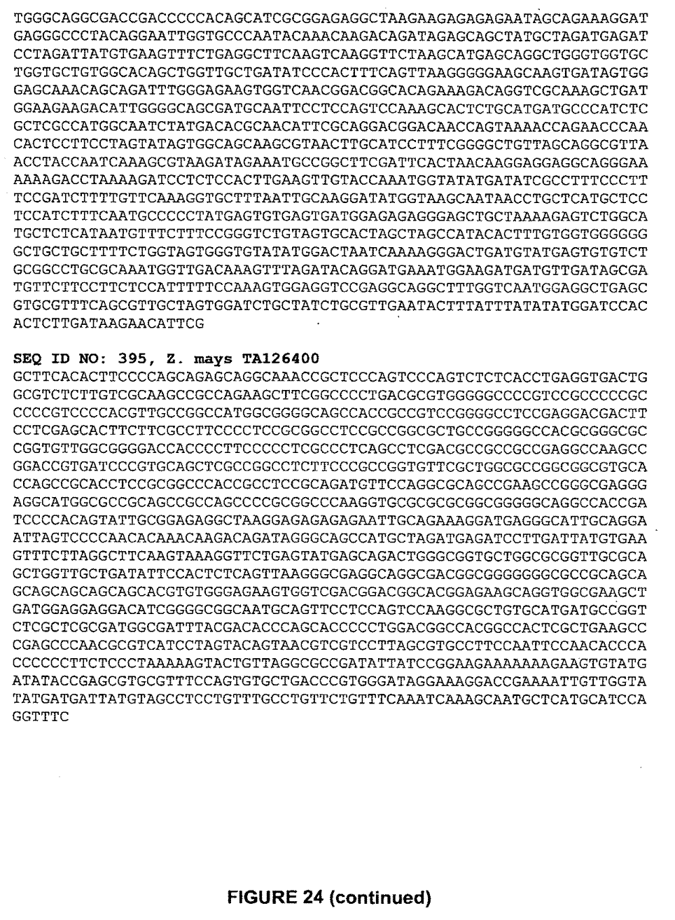

FIG. 24 details examples of bHLH11-like sequences useful in performing the methods according to the present invention.

### VI. ASR (Abscisic Acid-, Stress-, and Ripening-Induced) Protein

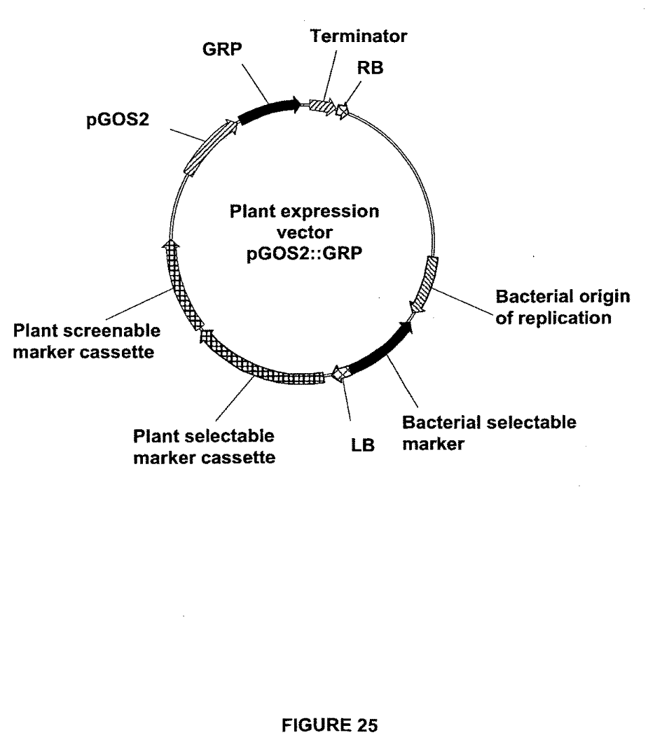

FIG. 25 represents the binary vector for increased expression in Oryza sativa of a GRP-encoding nucleic acid under the control of a rice GOS2 promoter (pGOS2::GRP).

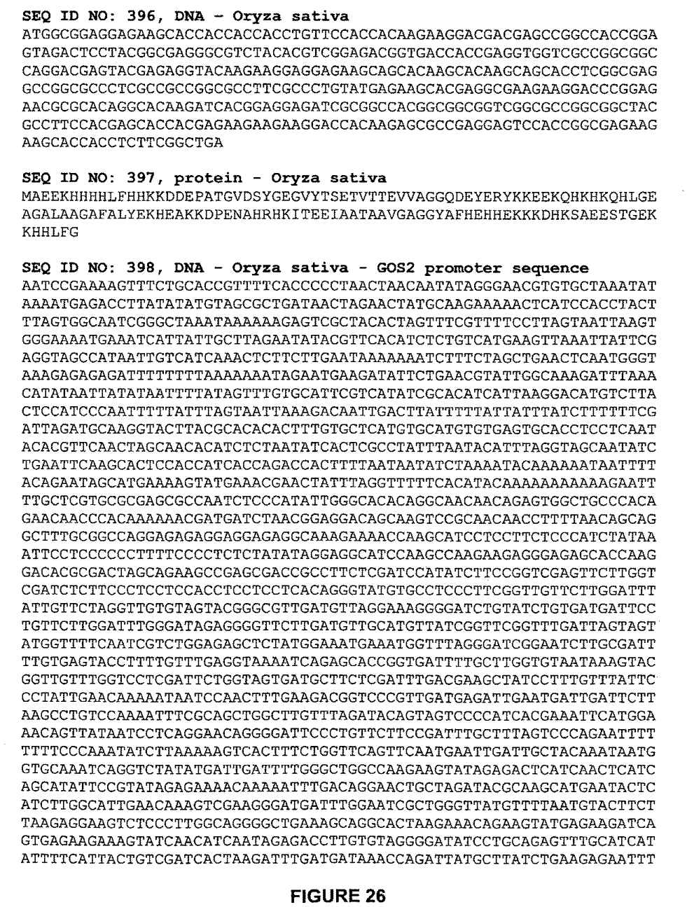

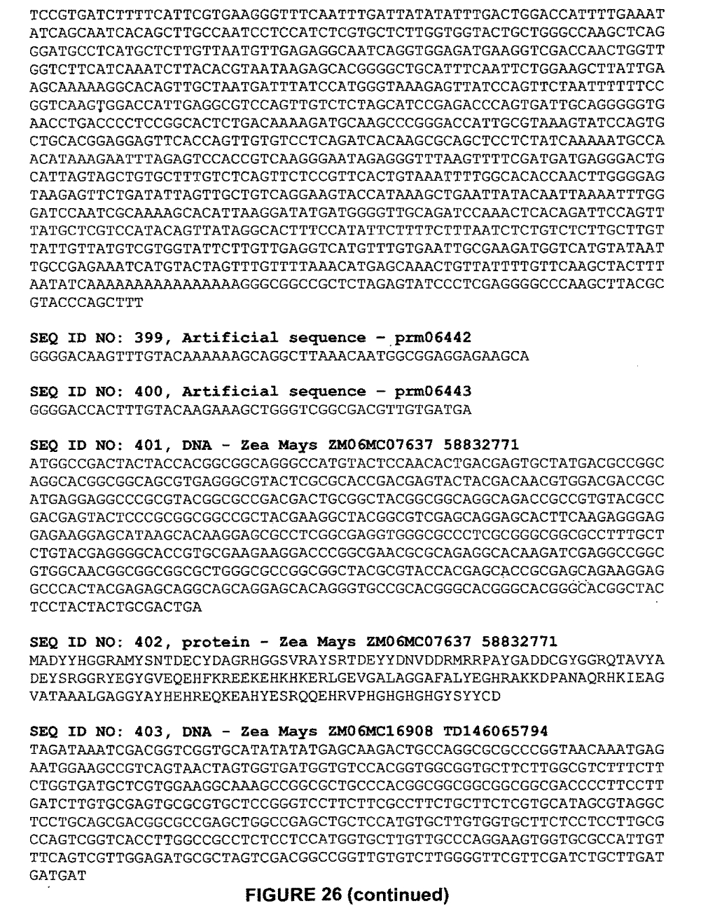

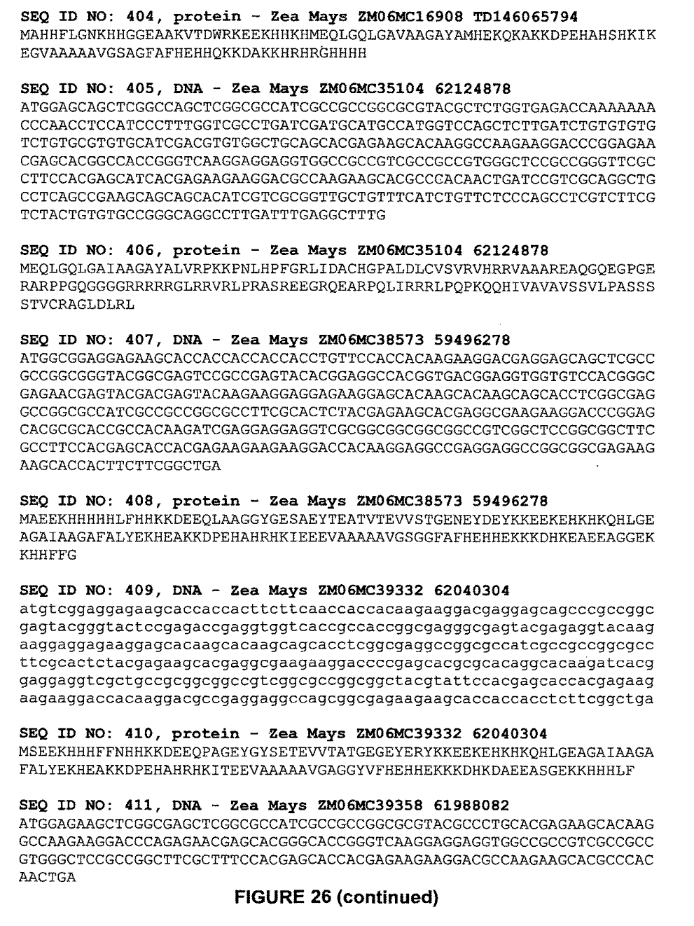

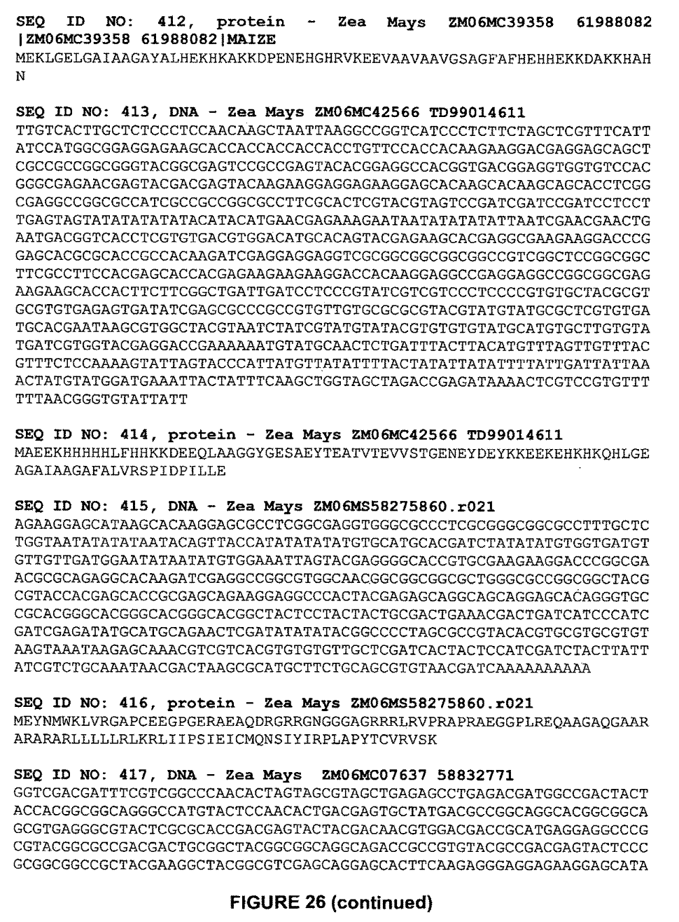

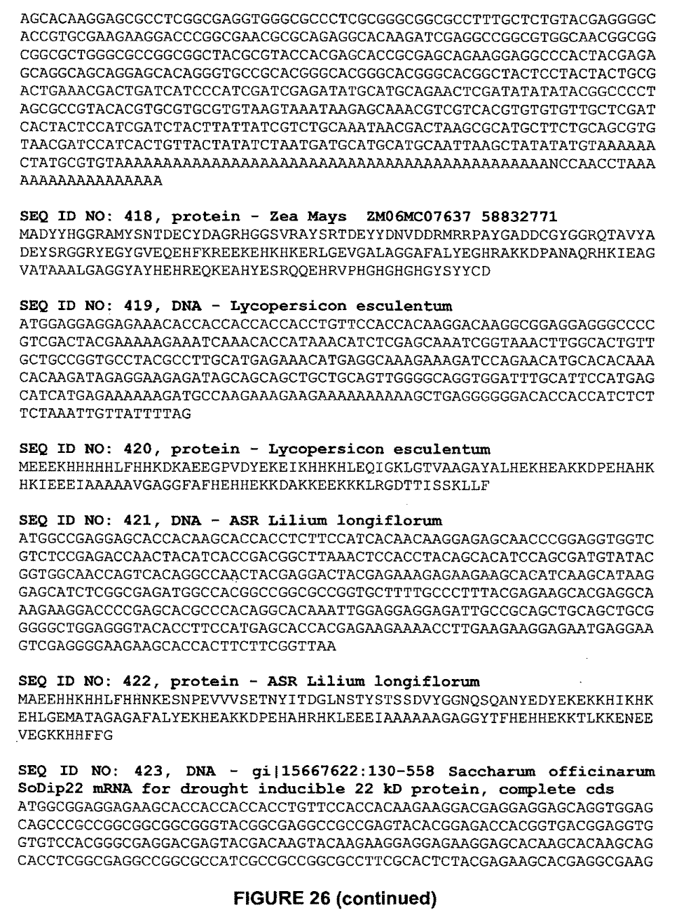

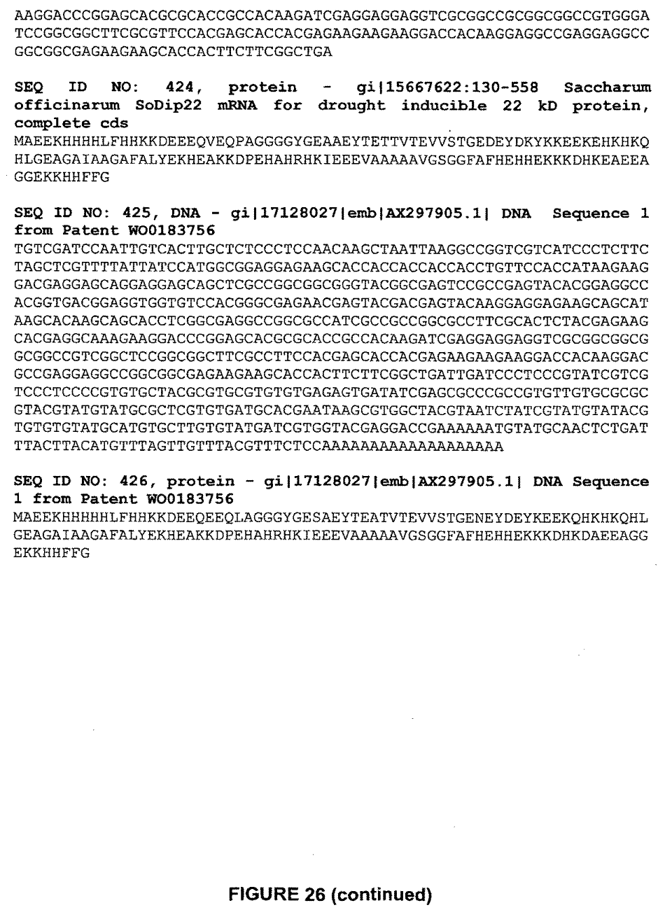

FIG. 26 details examples of GRP sequences useful in performing the methods according to the present invention.

### VII. Squamosa Promoter Binding Protein-Like 11 (SPL11)

FIG. 27 represents the amino acid sequence and domain structure of SEQ ID NO: 428. Conserved motifs and domains are indicated. SBP domain is underlined by an interrupted line; Motif 1 is indicated in bold; Motif 2 is indicated in bold and underlined; Motif 3 is bold capital letters and underlined and Motif 4 is in bold and underlined with a double line.

FIG. 28 represents a multiple sequence alignment of SPL11 polypeptides. Position of the conserved amino acid residues and domains corresponding to those described in FIG. 27 is indicated. A consensus sequence representative of SPL11 polypeptides is given. In the consensus sequence the highly conserved amino acids are provided and empty blank spaces in between represent any amino acid.

FIG. 29 shows a phylogenetic tree of SPL11 polypeptides. The phylogenetic tree is as present in Xie et al. Plant Physiology, 2006, Vol. 142, pp. 280-293, which is incorporated by reference herein as if fully set forth. SPL11 polypeptides cluster in the same group as AtSPL11 (identical to SEQ ID NO: 2) within the class named Class S3.

FIG. 30 provides a sequence analysis of rice miR156 genes (OsmiR156) and their targeted sequences in rice SPL polypeptides (FIG. 30 A) as well as a multiple alignment of SPL11 nucleic acids (FIG. 30 B). FIG. 30 A shows a sequence alignment of OsmiR156 mature sequences with complementary sequences of OsSPL genes. The conserved amino acid sequence encoded by the target sequences is shown at the bottom. The dots between miR156 and targeted OsSPL sequences indicate mismatches. FIG. 4B shows a multiple alignment of SPL11 nucleic acids where in highly conserved nucleic acid residues are indicated in the consensus sequence. The position of the highly conserved miR156 target site is indicated in bold over the consensus sequence. SEQ ID NO: 431, SEQ ID NO: 440 and SEQ ID NO: 454 representatives of SPL11 nucleic acids which are miR156 insensitive do not have the conserved miR156 target site or show a great divergence in their sequence at that position.

FIG. 31 represents the binary vector for increased expression in Oryza sativa of SEQ ID NO: 427 under the control of a rice GOS2 promoter (pGOS2) (FIG. 31A) or under a rice WSI 18 promoter (FIG. 318).

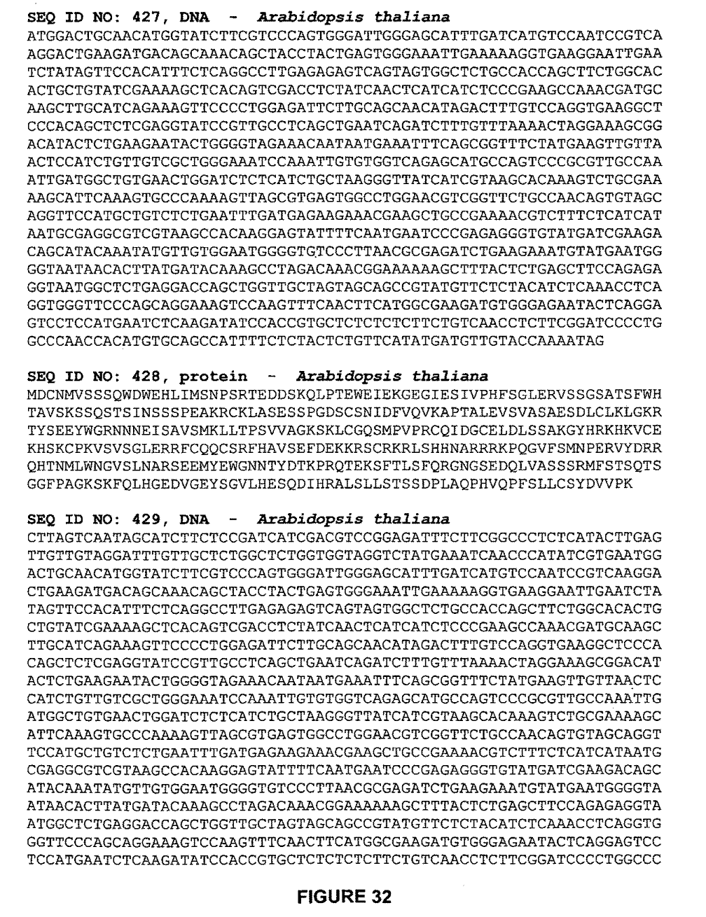

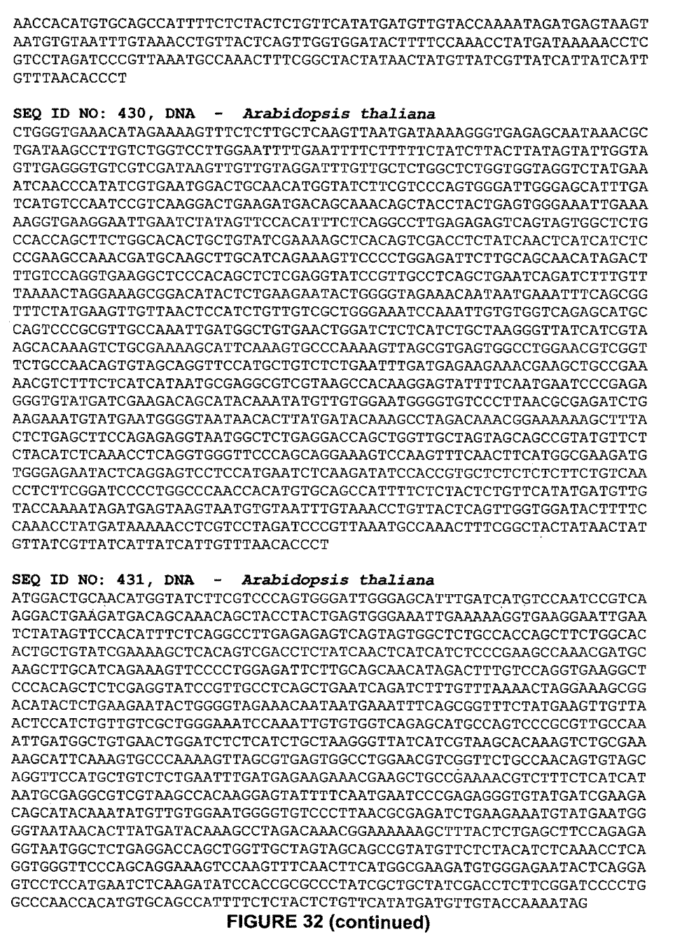

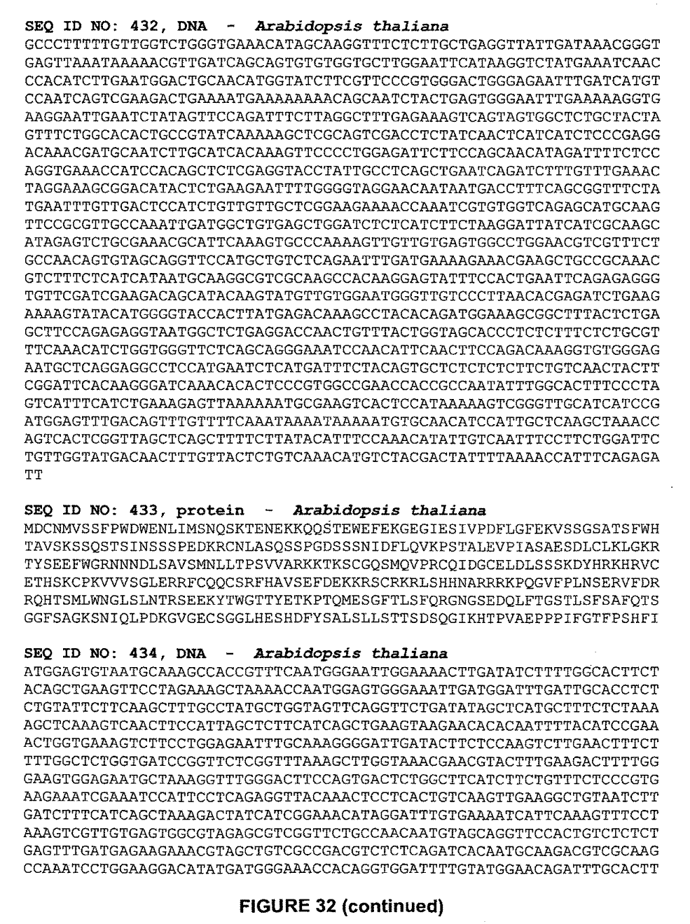

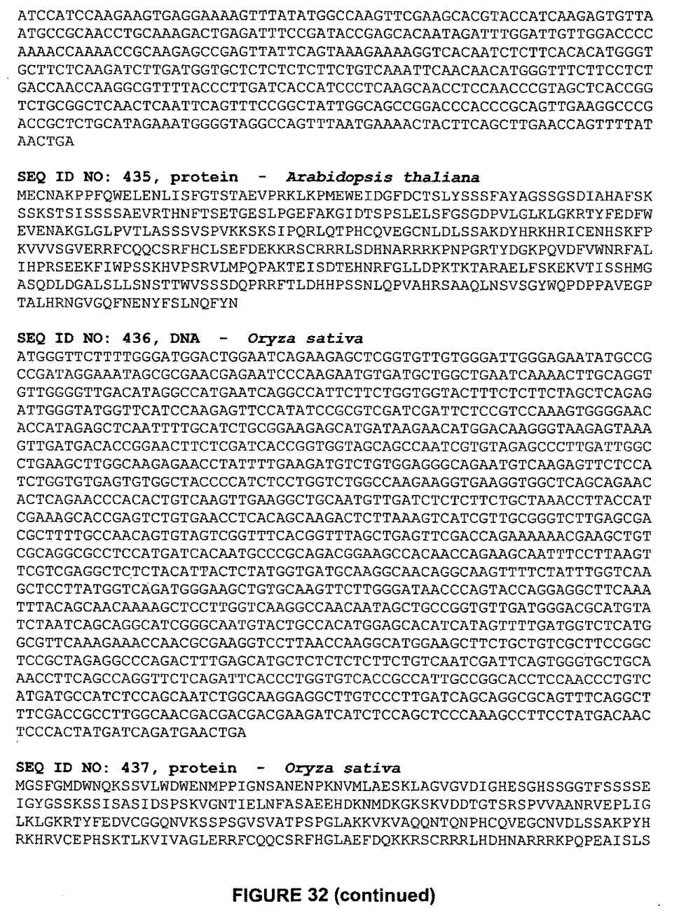

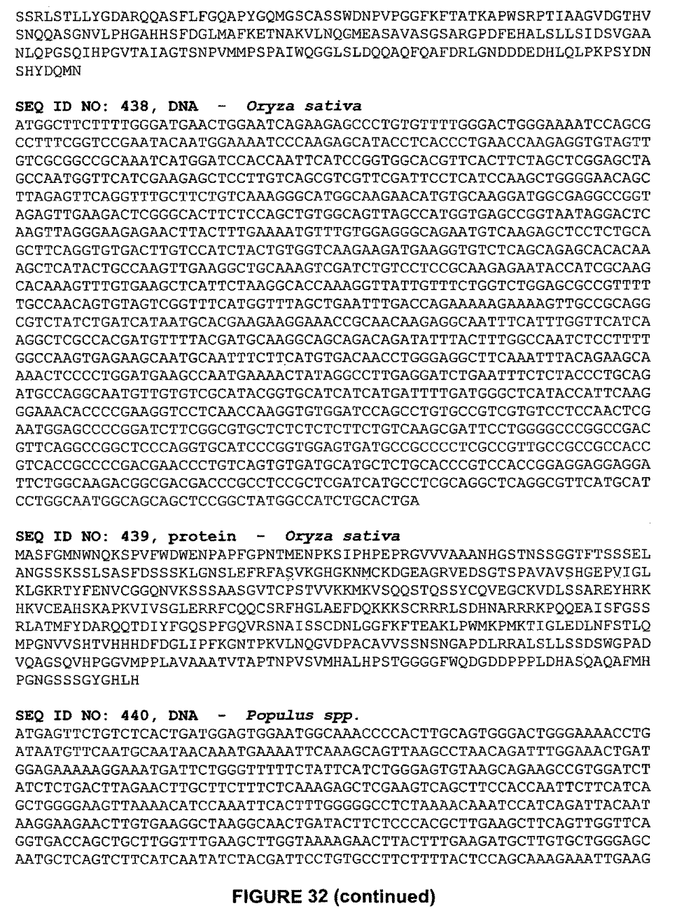

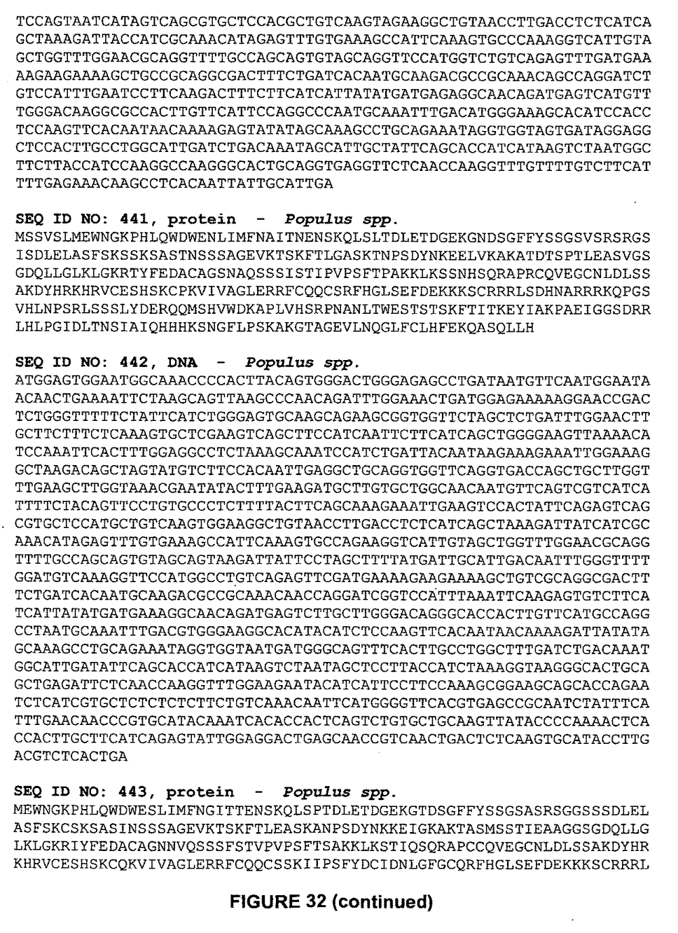

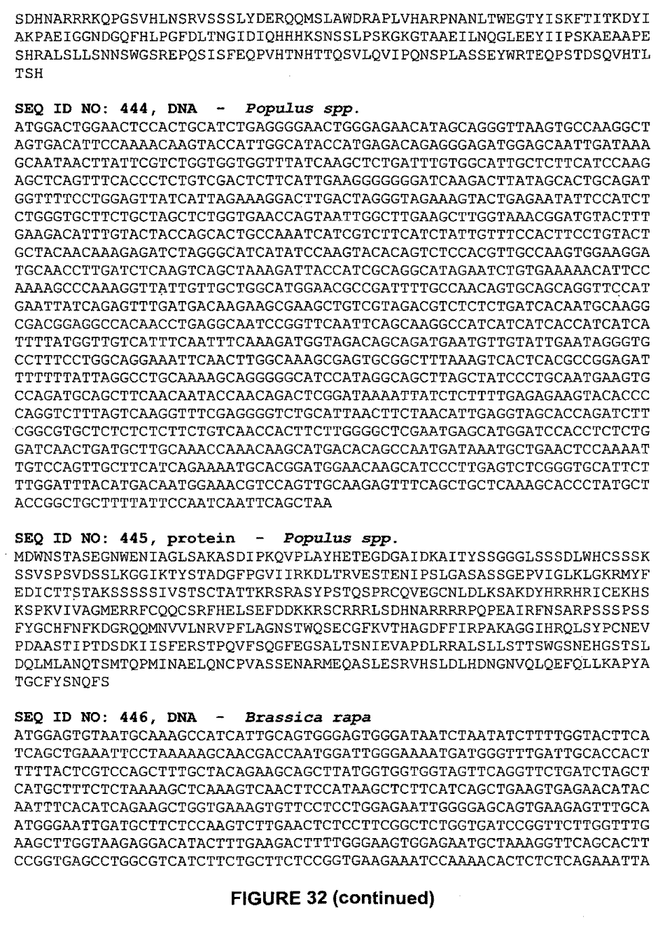

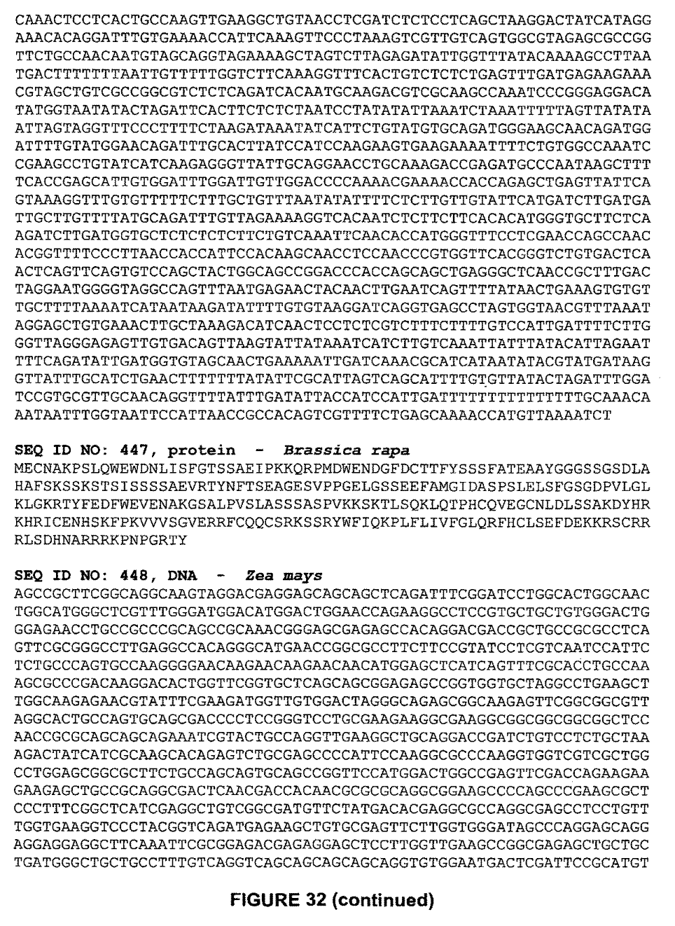

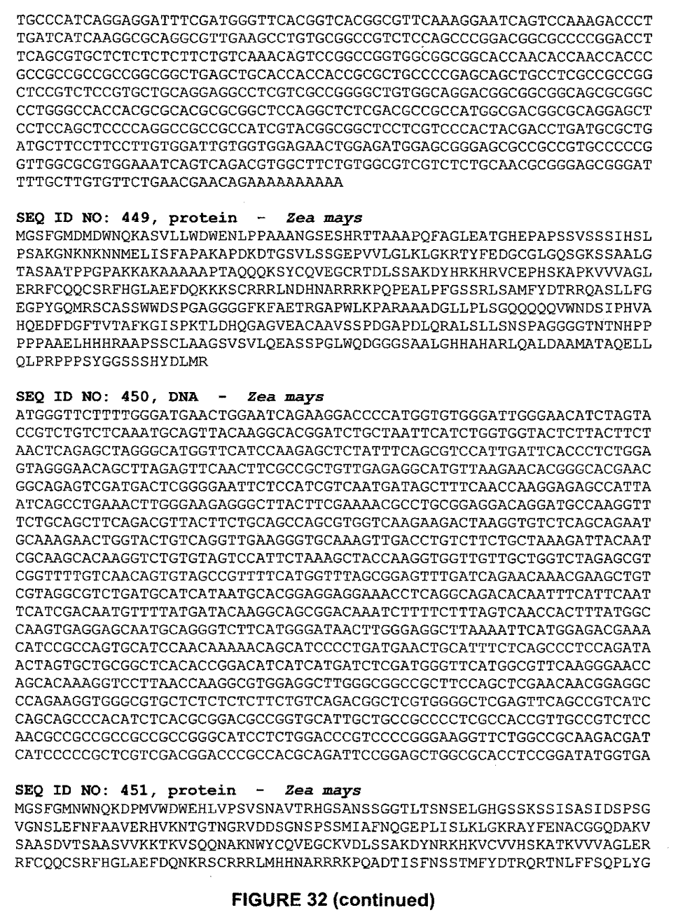

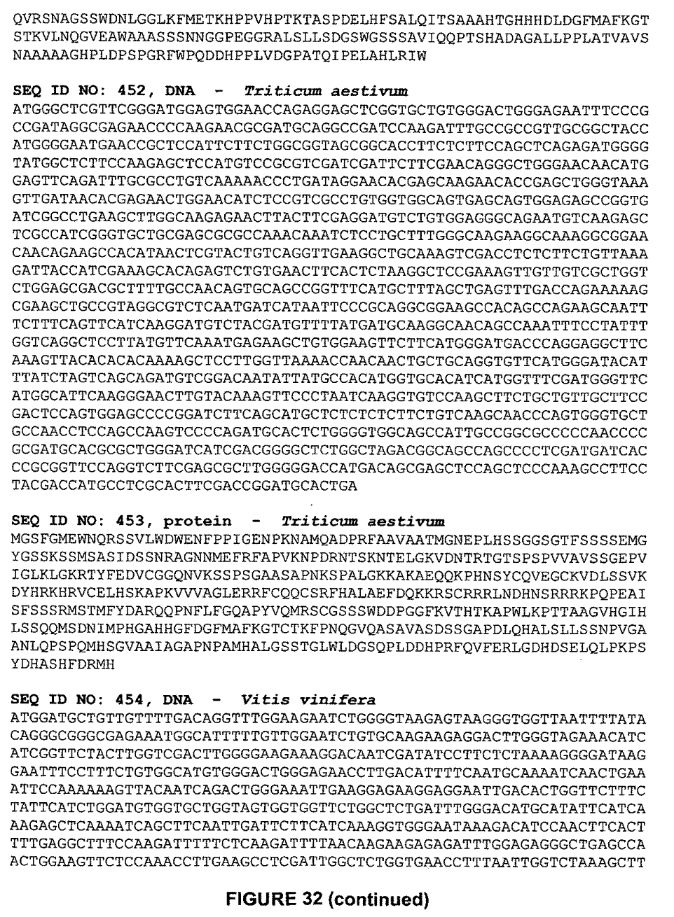

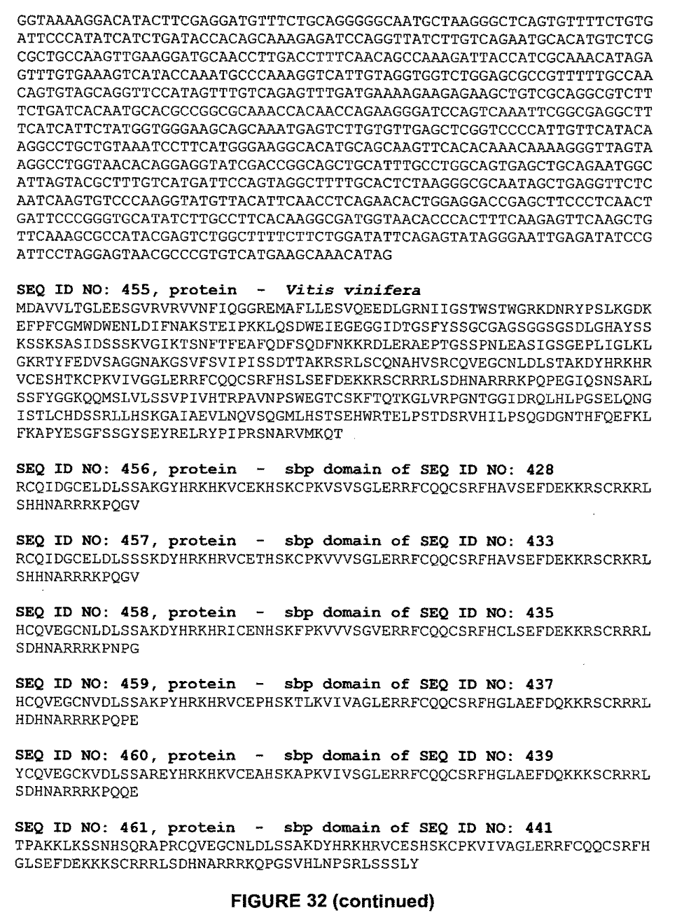

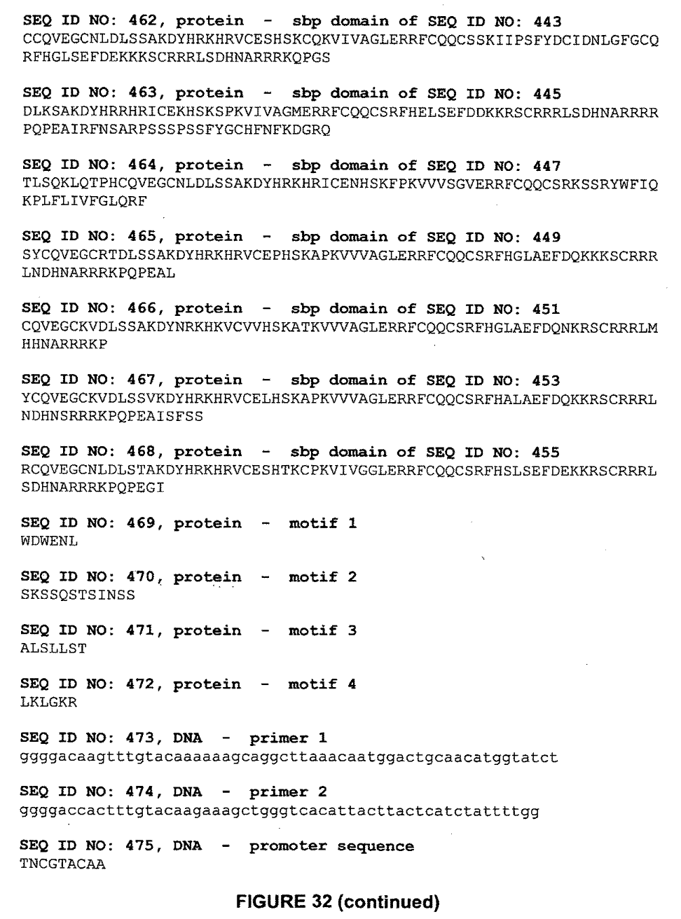

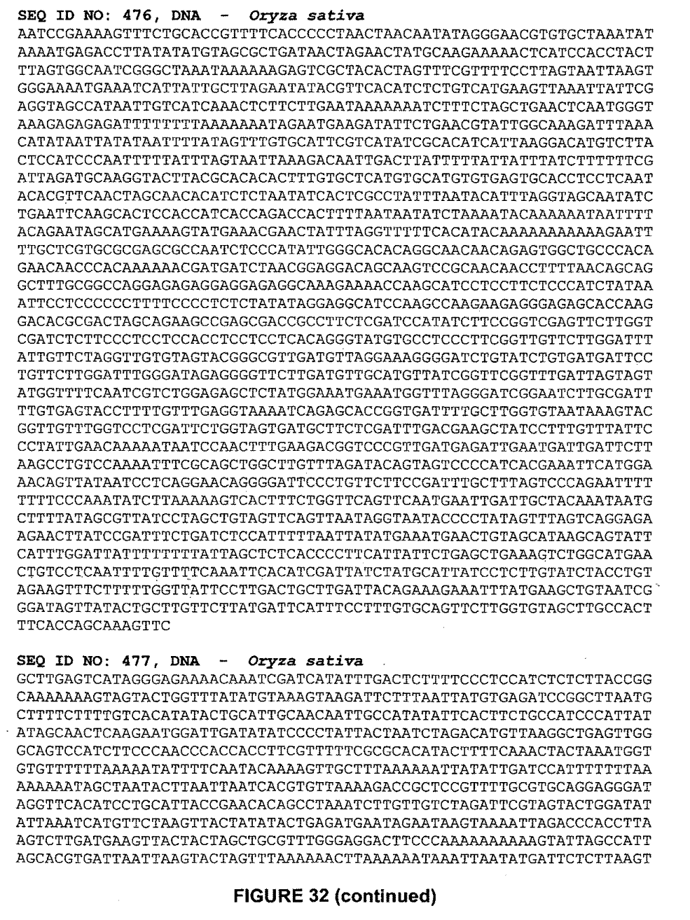

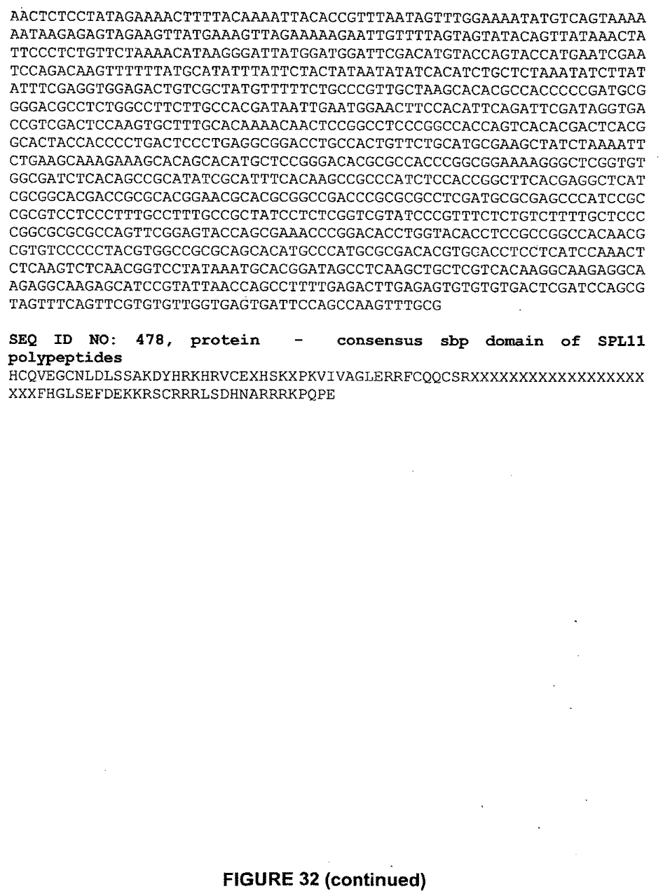

FIG. 32 details examples of SPL11 sequences useful in performing the methods according to the present invention.

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in Sambrook (2001) Molecular Cloning: a laboratory manual, 3rd Edition Cold Spring Harbor Laboratory Press, CSH, New York, or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### I. LOB-Domain Comprising Protein (LOB: Lateral Organ Boundaries)

### Example 1

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program was used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length.

Table A1 provides a list of nucleic acid sequences related to the LBD nucleic acid sequence used in the methods of the present invention.

TABLE A1
Examples of LBD polypeptides: add DNA sequences of rice,
corn, wheat, canola, potato, soy, Arabidopsis

| | Nucleic acid | Protein |
|---|---|---|
| Plant Source* | SEQ ID NO: | SEQ ID NO: |
| Arabidopsis thaliana LBD protein | 1 | 2 |
| Os01g03890, Oryza sativa | 59 | 11 |
| Os01g32770, Oryza sativa | 60 | 12 |
| Os03g33090, Oryza sativa | 61 | 13 |
| Os03g41330, Oryza sativa | 62 | 14 |
| Os07g40000, Oryza sativa | 63 | 15 |
| TC9404, Nicotiana benthamiana | | 16 |
| TC227562, Glycine max | | 17 |
| TC216138, Glycine max | | 18 |
| TC147776, Hordeum vulgare | | 19 |
| TC104758, Sorghum bicolor | | 20 |
| TC18561, Aquilegia sp. | | 21 |
| TC60668, Vitis vinifera | | 22 |
| TC15459, Lotus japonicus | | 23 |
| TC30552, Gossypium hirsutum | | 24 |
| TC235711, Triticum aestivum | 71 | 25 |
| TC133081, Solanum tuberosum | | 26 |
| TC107091, Medicago truncatula | | 27 |
| TC147808, Hordeum vulgare | | 28 |
| TC55931, Vitis vinifera | | 29 |
| TC162239, Solanum tuberosum | | 30 |
| TC69225, Pinus taeda | | 31 |
| TC67269, Pinus taeda | | 32 |
| TC220806, Glycine max | | 33 |
| TC270332, Triticum aestivum | | 34 |
| TC18329, Aquilegia sp. | | 35 |
| TC137193, Solanum tuberosum | | 36 |
| TC133385, Solanum tuberosum | | 37 |
| TC140088, Solanum tuberosum | | 38 |
| TC14656, Picea alba | | 39 |
| TC59178, Pinus taeda | | 40 |
| TC67974, Pinus taeda | | 41 |
| TC178827, Lycopersicon esculentum | | 42 |
| Pt-III.589, Populus tremuloides | | 43 |
| Pt-V.543, Populus tremuloides | | 44 |
| Pt-XIV.94, Populus tremuloides | | 45 |
| Pt-II105, Populus tremuloides | | 46 |
| Pt-123.86, Populus tremuloides | | 47 |
| Pt-X180, Populus tremuloides | | 48 |
| Pt-XII.481, Populus tremuloides | | 49 |
| DQ787782, Caragana korshinskii | | 50 |
| AAP37970, Brassica napus | | 51 |
| ABE82505, Medicago truncatula | 72 | 52 |
| ABE78739, Medicago truncatula | | 53 |
| Q9SN23, Arabidopsis thaliana | 64 | 54 |
| Q9SZE8, Arabidopsis thaliana | 65 | 55 |
| Q9ZW96, Arabidopsis thaliana | 66 | 56 |
| Q9M886, Arabidopsis thaliana | 67 | 57 |
| Q9CA30, Arabidopsis thaliana | 68 | 58 |
| Ls_LBD, Linum usitatissimum | 69 | 70 |

*GenBank or SwissProt database accession numbers are provided where available, TC codes are from TIGR.

### Example 2

### Alignment of LBD Polypeptide Sequences

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chema et al. (2003). Nucleic Acids Res 31:3497-3500). Default values were for the gap open penalty of 10, for the gap extension penalty of 0,1 and the selected weight matrix is Blosum 62. Minor manual editing was done to further optimise the alignment. Sequence conservation among LBD polypeptides was essentially in the N-terminal DUF260 domain of the polypeptides, the C-terminal region usually being more variable in sequence length and composition. The LBD polypeptides are aligned in FIG. 2.

A phylogenetic tree of LBD polypeptides (FIG. 3) was constructed using a neighbour-joining clustering algorithm as provided in the AlignX programme from the Vector NTI (Invitrogen). The sequences of class I LBD proteins used in the construction of the tree are publicly available and are indicated with their GenBank or SwissProt accession numbers.

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B for the global similarity and identity over the full length of the polypeptide sequences. Percentage identity is given above the diagonal in bold and percentage similarity is given below the diagonal (normal face).

The percentage identity between the LBD polypeptide sequences useful in performing the methods of the invention can be as low as 30.2% amino acid identity compared to SEQ ID NO: 2 (represented by At-LBD37, row 44). The % identity will likely be higher when only the sequences of the DUF206 domain are compared. To identify the DUF260 domain (as delineated in FIG. 1) in other LBD proteins, the multiple alignment of FIG. 2 may be used.

TABLE A2
MatGAT results for global similarity and identity over the full length of the LBD
polypeptide sequences.

| | 1 | 2 | 3 | 4 | 5 | 6 | 7 | 8 | 9 | 10 | 11 | 12 |
|---|---|---|---|---|---|---|---|---|---|---|---|---|
| 1. Os-Os01g03890 | | 43.2 | 35.8 | 34.5 | 34.4 | 32.9 | 33.0 | 35.5 | 35.2 | 34.5 | 31.5 | 32.2 |
| 2. Os-Os01g32770 | 55.5 | | 31.0 | 31.1 | 31.5 | 30.3 | 32.4 | 30.7 | 30.6 | 30.9 | 29.8 | 31.2 |
| 3. Os-Os03g33090 | 46.9 | 40.3 | | 48.3 | 65.0 | 56.7 | 53.9 | 52.1 | 66.7 | 71.3 | 51.5 | 55.0 |
| 4. Os-Os03g41330 | 46.3 | 43.0 | 60.3 | | 48.3 | 43.0 | 43.2 | 41.2 | 46.4 | 48.6 | 40.2 | 45.1 |
| 5. Os-Os07g40000 | 45.9 | 40.9 | 73.0 | 59.9 | | 53.0 | 50.6 | 49.6 | 68.0 | 63.1 | 47.6 | 52.3 |
| 6. Nb-TC9404 | 45.9 | 42.4 | 66.5 | 60.3 | 67.7 | | 60.7 | 60.3 | 51.7 | 51.5 | 56.7 | 66.5 |
| 7. Gm-TC227562 | 45.0 | 44.5 | 65.2 | 59.1 | 65.2 | 75.2 | | 73.7 | 50.6 | 49.4 | 55.2 | 64.8 |
| 8. Gm-TC216138 | 47.2 | 41.5 | 63.3 | 58.8 | 62.1 | 70.4 | 79.6 | | 50.0 | 48.8 | 53.3 | 65.3 |
| 9. Hv-TC147776 | 45.9 | 41.5 | 76.1 | 58.6 | 79.6 | 69.2 | 67.4 | 63.8 | | 58.7 | 47.3 | 52.2 |
| 10. Sb-TC104758 | 45.3 | 40.6 | 77.0 | 59.6 | 71.1 | 63.0 | 62.6 | 58.8 | 68.5 | | 49.0 | 51.7 |
| 11. Aq-TC18561 | 47.2 | 43.3 | 61.5 | 56.9 | 62.3 | 67.5 | 69.7 | 63.8 | 67.1 | 61.7 | | 58.1 |
| 12. Vv-TC60668 | 45.0 | 43.3 | 65.4 | 59.1 | 66.7 | 79.4 | 77.4 | 77.1 | 66.2 | 64.7 | 72.7 | |
| 13. Lj-TC15459 | 45.6 | 40.9 | 64.1 | 59.1 | 64.1 | 72.2 | 81.4 | 88.8 | 67.5 | 61.6 | 70.5 | 79.3 |
| 14. Gh-TC30552 | 46.3 | 43.0 | 66.8 | 63.4 | 64.7 | 75.9 | 78.4 | 75.4 | 66.8 | 64.3 | 72.8 | 82.8 |
| 15. Ta-TC235711 | 56.7 | 61.5 | 42.9 | 43.2 | 46.3 | 46.7 | 46.3 | 47.4 | 47.0 | 44.6 | 48.4 | 45.6 |
| 16. St-TC133081 | 59.0 | 55.8 | 47.4 | 48.5 | 47.1 | 50.7 | 49.6 | 47.4 | 49.3 | 48.9 | 50.4 | 50.0 |
| 17. Mt-TC107091 | 58.6 | 60.0 | 45.5 | 46.2 | 43.5 | 46.5 | 46.2 | 47.2 | 42.5 | 44.9 | 49.2 | 49.2 |
| 18. Hv-TC147808 | 59.0 | 64.8 | 42.1 | 42.1 | 44.7 | 44.4 | 46.7 | 46.0 | 45.0 | 44.7 | 44.4 | 45.0 |
| 19. Vv-TC55931 | 57.0 | 55.2 | 49.4 | 47.8 | 46.2 | 53.0 | 51.4 | 49.8 | 48.2 | 46.2 | 53.8 | 51.8 |
| 20. St-TC162239 | 49.5 | 48.2 | 50.4 | 52.6 | 51.7 | 54.7 | 54.7 | 55.0 | 46.6 | 51.1 | 57.7 | 56.8 |
| 21. Pta-TC69225 | 53.0 | 50.0 | 38.9 | 41.6 | 40.4 | 44.9 | 44.9 | 43.1 | 43.1 | 41.9 | 44.9 | 44.6 |
| 22. Pta-TC67269 | 51.8 | 52.7 | 39.6 | 41.4 | 39.3 | 42.6 | 42.9 | 41.4 | 43.2 | 40.8 | 44.0 | 42.6 |
| 23. Gm-TC220806 | 45.9 | 46.1 | 56.4 | 49.1 | 51.3 | 54.5 | 52.6 | 51.7 | 52.3 | 48.5 | 52.4 | 53.5 |
| 24. Ta-TC270332 | 41.4 | 40.6 | 56.5 | 84.5 | 59.1 | 61.3 | 59.1 | 57.1 | 60.0 | 57.9 | 59.7 | 58.7 |
| 25. Aq-TC18329 | 62.5 | 61.5 | 46.6 | 47.9 | 44.1 | 50.0 | 50.7 | 47.6 | 44.8 | 45.2 | 51.4 | 49.7 |
| 26. St-TC137193 | 45.6 | 42.1 | 68.7 | 57.3 | 65.9 | 89.3 | 72.6 | 70.4 | 68.9 | 61.3 | 74.0 | 77.6 |
| 27. St-TC133385 | 45.6 | 41.8 | 70.8 | 56.9 | 66.4 | 87.9 | 71.3 | 68.3 | 66.2 | 62.1 | 70.6 | 76.3 |
| 28. St-TC140088 | 44.3 | 42.1 | 64.5 | 51.3 | 61.5 | 71.0 | 68.3 | 65.8 | 65.8 | 60.9 | 68.4 | 70.2 |
| 29. Pa-TC14656 | 48.5 | 45.2 | 47.7 | 46.9 | 48.4 | 51.6 | 52.7 | 52.7 | 47.3 | 48.0 | 53.4 | 53.8 |
| 30. Pta-TC59178 | 47.7 | 45.2 | 40.5 | 40.2 | 43.5 | 44.1 | 43.3 | 44.6 | 44.1 | 40.5 | 46.8 | 46.6 |
| 31. Pta-TC67974 | 48.2 | 45.2 | 50.0 | 48.2 | 46.4 | 52.5 | 52.2 | 54.3 | 48.6 | 49.3 | 54.0 | 54.3 |
| 32. Le-TC178827 | 45.9 | 41.8 | 68.3 | 57.8 | 66.8 | 89.7 | 73.9 | 71.7 | 67.6 | 62.1 | 73.6 | 78.1 |
| 33. Pt-III.589 | 45.0 | 43.9 | 63.0 | 60.1 | 62.6 | 73.5 | 76.5 | 79.6 | 68.5 | 64.3 | 69.7 | 79.0 |
| 34. Pt-V.543 | 48.2 | 43.9 | 64.5 | 60.3 | 60.7 | 74.0 | 76.4 | 77.7 | 66.5 | 63.6 | 68.6 | 77.3 |
| 35. Pt-XIV.94 | 41.7 | 39.4 | 64.4 | 56.0 | 58.0 | 63.4 | 61.3 | 58.8 | 61.3 | 55.7 | 64.9 | 64.5 |
| 36. Pt-II105 | 43.0 | 39.1 | 63.9 | 54.7 | 58.0 | 60.7 | 63.0 | 58.8 | 65.3 | 56.6 | 63.6 | 65.8 |
| 37. Pt-123.86 | 57.7 | 57.9 | 47.6 | 46.3 | 46.3 | 46.6 | 48.6 | 47.6 | 43.6 | 45.9 | 47.3 | 45.9 |
| 38. Pt-X180 | 52.1 | 49.4 | 50.2 | 51.0 | 49.8 | 54.8 | 55.6 | 55.2 | 48.5 | 50.2 | 56.0 | 57.7 |
| 39. Pt-XII.481 | 51.5 | 47.3 | 43.6 | 45.8 | 44.7 | 48.4 | 46.5 | 49.5 | 46.5 | 46.9 | 46.9 | 50.9 |
| 40. Ck-LOB-DQ787782 | 48.5 | 43.3 | 66.7 | 59.5 | 60.6 | 73.2 | 82.3 | 82.1 | 65.8 | 61.7 | 71.4 | 77.5 |
| 41. Bn-AAP37970 | 56.0 | 56.1 | 48.9 | 49.6 | 46.2 | 49.6 | 50.4 | 51.9 | 50.0 | 47.0 | 50.0 | 51.5 |
| 42. Mt-ABE82505 | 47.2 | 44.2 | 65.2 | 62.2 | 65.2 | 72.5 | 79.8 | 80.8 | 64.8 | 64.3 | 71.7 | 76.8 |
| 43. Mt-ABE78739 | 54.7 | 56.7 | 45.7 | 48.9 | 44.6 | 50.7 | 50.4 | 49.6 | 47.1 | 47.8 | 51.1 | 50.4 |
| 44. At-LBD37-Q9FN11 | 45.0 | 46.1 | 61.6 | 56.4 | 64.0 | 68.8 | 68.8 | 69.6 | 64.0 | 63.6 | 64.0 | 68.8 |
| 45. At-LBD38-Q9SN23 | 45.9 | 45.2 | 61.9 | 55.5 | 63.2 | 61.9 | 70.4 | 70.4 | 62.8 | 63.6 | 67.6 | 70.9 |
| 46. At-LBD39-Q9SZE8 | 45.0 | 41.8 | 61.3 | 56.7 | 63.3 | 65.8 | 70.8 | 67.9 | 60.0 | 62.5 | 70.4 | 71.7 |
| 47. At-LBD40-Q9ZW96 | 53.1 | 49.1 | 54.5 | 54.1 | 55.8 | 55.8 | 54.9 | 52.5 | 53.2 | 56.2 | 60.9 | 56.7 |
| 48. At-LBD41-Q9M886 | 52.8 | 56.7 | 44.9 | 49.0 | 47.5 | 49.8 | 50.6 | 47.9 | 49.8 | 47.1 | 51.0 | 50.2 |
| 49. At-LBD42-Q9CA30 | 52.4 | 49.4 | 54.1 | 50.2 | 51.9 | 58.4 | 57.5 | 52.9 | 54.1 | 52.8 | 55.4 | 55.4 |
| | 13 | 14 | 15 | 16 | 17 | 18 | 19 | 20 | 21 | 22 | 23 | 24 |
| 1. Os-Os01g03890 | 33.7 | 33.0 | 42.5 | 46.6 | 42.9 | 43.5 | 42.8 | 37.7 | 36.0 | 36.8 | 38.1 | 32.2 |
| 2. Os-Os01g32770 | 30.1 | 32.4 | 52.2 | 46.5 | 47.1 | 54.4 | 41.7 | 37.0 | 37.1 | 38.2 | 39.7 | 30.9 |
| 3. Os-Os03g33090 | 54.0 | 54.5 | 32.4 | 37.0 | 34.4 | 31.3 | 37.2 | 37.6 | 30.2 | 28.3 | 41.3 | 47.4 |
| 4. Os-Os03g41330 | 41.2 | 44.1 | 32.1 | 33.1 | 30.2 | 31.4 | 36.4 | 37.0 | 29.9 | 28.4 | 35.5 | 78.7 |
| 5. Os-Os07g40000 | 50.0 | 51.7 | 34.7 | 35.8 | 33.9 | 33.2 | 35.2 | 36.5 | 28.7 | 28.3 | 38.7 | 46.3 |
| 6. Nb-TC9404 | 60.8 | 63.7 | 33.7 | 35.8 | 33.2 | 32.1 | 38.7 | 38.1 | 32.3 | 29.8 | 39.6 | 44.0 |
| 7. Gm-TC227562 | 72.4 | 67.6 | 33.1 | 34.7 | 33.2 | 33.1 | 36.6 | 36.2 | 32.3 | 30.7 | 39.3 | 42.8 |
| 8. Gm-TC216138 | 81.5 | 66.5 | 34.4 | 34.2 | 33.2 | 33.2 | 37.0 | 35.7 | 31.1 | 30.7 | 37.6 | 42.3 |
| 9. Hv-TC147776 | 50.8 | 51.9 | 34.9 | 35.4 | 31.6 | 32.6 | 36.8 | 34.6 | 29.3 | 28.6 | 37.9 | 45.4 |
| 10. Sb-TC104758 | 49.4 | 51.3 | 33.8 | 34.5 | 32.5 | 33.1 | 35.3 | 38.0 | 29.6 | 30.1 | 37.4 | 46.9 |
| 11. Aq-TC18561 | 54.8 | 58.5 | 33.4 | 35.0 | 34.2 | 32.1 | 37.5 | 37.6 | 32.3 | 30.8 | 37.3 | 41.1 |
| 12. Vv-TC60668 | 65.6 | 68.3 | 33.1 | 35.6 | 35.9 | 32.1 | 36.2 | 38.5 | 33.3 | 32.0 | 39.6 | 43.5 |
| 13. Lj-TC15459 | | 64.9 | 34.5 | 33.2 | 33.8 | 33.4 | 36.0 | 36.5 | 34.1 | 29.4 | 36.8 | 39.6 |
| 14. Gh-TC30552 | 76.8 | | 36.2 | 35.4 | 33.9 | 34.1 | 37.5 | 36.9 | 32.0 | 30.7 | 40.1 | 44.2 |
| 15. Ta-TC235711 | 47.4 | 49.5 | | 45.5 | 49.5 | 89.9 | 49.1 | 41.3 | 37.8 | 36.4 | 46.0 | 31.8 |
| 16. St-TC133081 | 50.0 | 50.4 | 57.5 | | 56.6 | 47.4 | 52.5 | 45.4 | 37.4 | 40.1 | 50.0 | 32.5 |
| 17. Mt-TC107091 | 48.2 | 47.5 | 61.5 | 66.4 | | 50.5 | 48.8 | 41.6 | 36.4 | 39.5 | 51.7 | 31.2 |
| 18. Hv-TC147808 | 45.4 | 47.7 | 92.4 | 59.6 | 63.2 | | 48.3 | 39.0 | 37.8 | 39.1 | 43.7 | 31.8 |
| 19. Vv-TC55931 | 51.8 | 51.4 | 60.3 | 63.1 | 61.1 | 57.9 | | 47.0 | 35.8 | 38.1 | 51.2 | 36.4 |
| 20. St-TC162239 | 54.9 | 55.1 | 55.7 | 58.8 | 52.8 | 52.3 | 63.2 | | 32.9 | 36.0 | 48.7 | 37.7 |
| 21. Pta-TC69225 | 46.4 | 44.0 | 50.9 | 51.5 | 53.0 | 51.2 | 47.9 | 43.1 | | 44.5 | 32.9 | 27.8 |
| 22. Pta-TC67269 | 42.3 | 44.3 | 50.9 | 54.8 | 52.4 | 52.4 | 50.0 | 46.4 | 63.1 | | 35.3 | 28.6 |
| 23. Gm-TC220806 | 51.1 | 54.7 | 51.6 | 55.8 | 55.5 | 49.0 | 57.3 | 60.7 | 40.1 | 42.0 | | 35.8 |
| 24. Ta-TC270332 | 54.9 | 61.2 | 44.3 | 45.3 | 44.2 | 42.7 | 47.8 | 50.9 | 41.0 | 40.2 | 51.7 | |
| 25. Aq-TC18329 | 49.7 | 51.0 | 63.8 | 70.0 | 70.8 | 65.2 | 65.9 | 55.5 | 52.1 | 53.6 | 54.1 | 45.9 |
| 26. St-TC137193 | 71.3 | 74.6 | 46.0 | 48.9 | 48.5 | 45.4 | 52.6 | 52.1 | 43.1 | 41.7 | 57.1 | 58.7 |
| 27. St-TC133385 | 70.0 | 73.3 | 43.9 | 48.9 | 46.5 | 43.0 | 52.6 | 53.0 | 41.9 | 41.4 | 57.1 | 57.0 |
| 28. St-TC140088 | 66.7 | 70.3 | 45.3 | 47.4 | 46.8 | 43.0 | 53.0 | 52.6 | 42.5 | 41.1 | 55.3 | 52.2 |
| 29. Pa-TC14656 | 51.6 | 53.4 | 50.2 | 49.8 | 48.2 | 50.0 | 46.6 | 52.3 | 47.3 | 46.1 | 44.8 | 44.8 |
| 30. Pta-TC59178 | 44.4 | 45.7 | 41.6 | 44.9 | 46.6 | 44.9 | 44.4 | 42.7 | 47.4 | 49.6 | 39.1 | 40.2 |
| 31. Pta-TC67974 | 50.7 | 54.3 | 49.1 | 50.0 | 48.2 | 47.0 | 47.8 | 51.8 | 46.4 | 48.2 | 46.7 | 47.8 |
| 32. Le-TC178827 | 72.6 | 75.4 | 46.7 | 48.9 | 48.2 | 43.7 | 53.0 | 52.1 | 42.5 | 43.2 | 56.9 | 58.3 |
| 33. Pt-III.589 | 78.6 | 81.9 | 50.5 | 51.5 | 48.5 | 47.0 | 55.3 | 57.1 | 44.3 | 44.6 | 53.4 | 58.0 |
| 34. Pt-V.543 | 76.9 | 79.8 | 49.1 | 52.2 | 49.2 | 48.0 | 53.0 | 56.2 | 45.2 | 45.5 | 53.3 | 57.0 |
| 35. Pt-XIV.94 | 59.9 | 62.5 | 44.9 | 45.6 | 42.5 | 44.0 | 48.2 | 51.7 | 41.6 | 41.4 | 56.3 | 57.8 |
| 36. Pt-II105 | 61.6 | 62.5 | 43.9 | 47.4 | 43.5 | 42.1 | 47.4 | 55.1 | 38.9 | 40.8 | 56.7 | 55.2 |
| 37. Pt-123.86 | 47.6 | 48.3 | 56.1 | 67.2 | 75.4 | 55.3 | 61.8 | 54.7 | 53.3 | 51.5 | 55.1 | 43.9 |
| 38. Pt-X180 | 56.0 | 56.8 | 53.0 | 56.6 | 55.1 | 51.0 | 66.4 | 59.8 | 48.8 | 47.6 | 60.2 | 49.8 |
| 39. Pt-XII.481 | 48.4 | 50.2 | 52.3 | 58.0 | 53.8 | 49.7 | 58.2 | 54.6 | 45.5 | 49.1 | 51.3 | 44.0 |
| 40. Ck-LOB-DQ787782 | 80.2 | 79.3 | 49.1 | 51.8 | 46.5 | 47.0 | 53.0 | 54.3 | 43.7 | 44.9 | 53.2 | 58.0 |
| 41. Bn-AAP37970 | 47.0 | 51.1 | 58.9 | 65.7 | 67.4 | 56.0 | 60.2 | 57.6 | 47.6 | 48.5 | 56.8 | 47.3 |
| 42. Mt-ABE82505 | 78.9 | 77.7 | 50.2 | 50.7 | 47.8 | 48.7 | 54.2 | 54.3 | 44.9 | 45.8 | 52.8 | 61.8 |
| 43. Mt-ABE78739 | 53.6 | 50.4 | 59.2 | 66.9 | 71.8 | 60.3 | 64.4 | 54.0 | 52.4 | 49.7 | 56.5 | 46.4 |
| 44. At-LBD37-Q9FN11 | 67.6 | 75.2 | 48.8 | 54.0 | 49.5 | 47.0 | 54.9 | 55.6 | 45.2 | 44.6 | 52.0 | 56.4 |
| 45. At-LBD38-Q9SN23 | 63.6 | 73.7 | 48.4 | 51.1 | 48.8 | 47.0 | 51.8 | 55.1 | 46.4 | 46.1 | 52.2 | 53.0 |
| 46. At-LBD39-Q9SZE8 | 73.8 | 70.0 | 48.8 | 50.4 | 47.8 | 47.4 | 52.6 | 53.8 | 45.2 | 43.8 | 51.7 | 61.7 |
| 47. At-LBD40-Q9ZW96 | 53.2 | 56.2 | 54.4 | 61.7 | 62.1 | 51.3 | 60.9 | 61.5 | 48.5 | 48.2 | 62.2 | 52.8 |
| 48. At-LBD41-Q9M886 | 49.8 | 52.1 | 56.8 | 67.5 | 67.4 | 57.0 | 61.2 | 58.9 | 49.1 | 49.1 | 57.4 | 46.0 |
| 49. At-LBD42-Q9CA30 | 56.1 | 54.5 | 54.0 | 54.7 | 54.5 | 51.3 | 60.5 | 64.5 | 47.0 | 45.8 | 62.7 | 51.1 |
| | 25 | 26 | 27 | 28 | 29 | 30 | 31 | 32 | 33 | 34 | 35 | 36 |
| 1. Os-Os01g03890 | 44.0 | 32.9 | 33.6 | 32.2 | 32.4 | 32.5 | 32.1 | 33.5 | 33.7 | 33.7 | 33.2 | 33.2 |
| 2. Os-Os01g32770 | 46.3 | 30.0 | 29.4 | 29.7 | 28.1 | 29.4 | 30.4 | 30.9 | 30.6 | 29.1 | 30.0 | 31.5 |
| 3. Os-Os03g33090 | 33.3 | 61.0 | 60.2 | 50.0 | 35.7 | 33.9 | 38.7 | 60.0 | 52.9 | 52.9 | 50.2 | 51.7 |
| 4. Os-Os03g41330 | 31.7 | 44.0 | 44.0 | 39.0 | 36.7 | 30.2 | 36.1 | 44.7 | 41.0 | 41.0 | 41.0 | 39.7 |
| 5. Os-Os07g40000 | 34.1 | 52.6 | 52.5 | 46.8 | 35.3 | 33.3 | 37.5 | 51.9 | 49.6 | 48.6 | 46.5 | 46.0 |
| 6. Nb-TC9404 | 36.6 | 83.2 | 82.3 | 57.5 | 38.9 | 36.9 | 41.9 | 83.2 | 64.2 | 64.9 | 50.2 | 49.1 |
| 7. Gm-TC227562 | 35.2 | 61.1 | 60.3 | 51.0 | 39.8 | 36.6 | 41.1 | 62.1 | 65.3 | 64.5 | 48.5 | 48.5 |
| 8. Gm-TC216138 | 34.1 | 61.4 | 60.2 | 51.7 | 38.2 | 34.9 | 40.2 | 61.7 | 67.2 | 66.8 | 47.5 | 46.9 |
| 9. Hv-TC147776 | 32.8 | 54.2 | 54.6 | 50.2 | 33.8 | 32.7 | 35.7 | 53.6 | 50.0 | 49.8 | 46.2 | 47.3 |
| 10. Sb-TC104758 | 31.4 | 51.7 | 51.5 | 46.7 | 35.6 | 33.1 | 37.5 | 49.0 | 52.5 | 50.2 | 43.8 | 45.1 |
| 11. Aq-TC18561 | 35.5 | 59.3 | 58.8 | 52.8 | 38.3 | 36.4 | 40.5 | 58.0 | 56.8 | 59.4 | 52.6 | 48.5 |
| 12. Vv-TC60668 | 35.1 | 67.1 | 67.2 | 54.5 | 39.6 | 36.6 | 42.3 | 66.5 | 69.4 | 66.7 | 50.9 | 50.4 |
| 13. Lj-TC15459 | 33.4 | 59.7 | 59.2 | 51.0 | 37.4 | 37.6 | 39.2 | 60.7 | 65.6 | 64.9 | 47.9 | 47.9 |
| 14. Gh-TC30552 | 35.2 | 63.9 | 63.4 | 57.3 | 39.4 | 37.2 | 41.6 | 64.2 | 71.4 | 70.9 | 50.0 | 50.0 |
| 15. Ta-TC235711 | 50.3 | 33.8 | 32.8 | 32.4 | 29.5 | 31.0 | 30.8 | 34.5 | 36.2 | 34.8 | 33.4 | 34.5 |
| 16. St-TC133081 | 57.4 | 33.2 | 34.7 | 32.5 | 30.9 | 32.1 | 32.6 | 35.4 | 36.4 | 36.0 | 35.0 | 34.2 |
| 17. Mt-TC107091 | 56.8 | 34.2 | 33.6 | 33.1 | 31.5 | 32.0 | 31.0 | 33.9 | 33.8 | 35.2 | 29.6 | 30.8 |
| 18. Hv-TC147808 | 50.6 | 34.4 | 31.5 | 29.1 | 31.4 | 31.6 | 30.3 | 33.1 | 34.8 | 34.8 | 32.1 | 32.5 |
| 19. Vv-TC55931 | 56.3 | 37.5 | 39.2 | 36.6 | 33.8 | 31.2 | 34.9 | 38.4 | 37.3 | 35.5 | 36.8 | 35.9 |
| 20. St-TC162239 | 41.8 | 36.3 | 37.2 | 35.0 | 35.6 | 32.0 | 35.7 | 37.6 | 39.0 | 37.3 | 37.6 | 37.9 |
| 21. Pta-TC69225 | 36.9 | 30.5 | 31.6 | 29.6 | 30.5 | 30.7 | 31.2 | 29.9 | 31.7 | 31.8 | 31.1 | 30.2 |
| 22. Pta-TC67269 | 43.1 | 30.1 | 29.2 | 29.2 | 28.5 | 30.0 | 32.2 | 31.5 | 29.5 | 31.0 | 29.8 | 29.8 |
| 23. Gm-TC220806 | 50.0 | 43.4 | 42.1 | 38.4 | 34.7 | 28.1 | 35.5 | 42.8 | 39.6 | 38.5 | 40.5 | 41.9 |
| 24. Ta-TC270332 | 32.1 | 45.5 | 43.5 | 39.2 | 35.7 | 30.3 | 36.8 | 43.3 | 41.3 | 40.9 | 40.9 | 39.7 |
| 25. Aq-TC18329 | | 35.5 | 35.2 | 31.4 | 30.2 | 33.1 | 31.5 | 34.8 | 33.4 | 35.5 | 33.4 | 34.1 |
| 26. St-TC137193 | 48.6 | | 96.8 | 58.4 | 38.8 | 36.1 | 41.9 | 95.0 | 64.3 | 62.5 | 50.2 | 50.2 |
| 27. St-TC133385 | 48.6 | 96.8 | | 59.7 | 38.1 | 35.8 | 40.1 | 92.8 | 63.9 | 60.9 | 50.7 | 50.9 |
| 28. St-TC140088 | 47.9 | 72.4 | 71.9 | | 38.8 | 33.2 | 39.8 | 58.7 | 54.1 | 53.8 | 46.3 | 45.6 |
| 29. Pa-TC14656 | 49.7 | 51.6 | 51.6 | 54.2 | | 38.6 | 88.1 | 38.7 | 42.1 | 40.3 | 33.5 | 33.5 |
| 30. Pta-TC59178 | 46.0 | 44.9 | 44.6 | 43.3 | 54.3 | | 39.4 | 36.9 | 36.4 | 38.4 | 32.5 | 31.4 |
| 31. Pta-TC67974 | 49.3 | 52.2 | 51.4 | 52.9 | 92.8 | 53.2 | | 41.7 | 42.5 | 42.8 | 35.0 | 36.1 |
| 32. Le-TC178827 | 48.3 | 97.2 | 95.0 | 71.6 | 50.9 | 45.2 | 51.4 | | 64.6 | 62.2 | 49.5 | 49.5 |
| 33. Pt-III.589 | 49.7 | 73.5 | 72.3 | 65.5 | 54.2 | 44.9 | 56.5 | 75.2 | | 83.3 | 50.4 | 48.7 |
| 34. Pt-V.543 | 50.3 | 73.6 | 71.5 | 64.0 | 54.2 | 46.8 | 56.2 | 73.1 | 88.8 | | 50.8 | 48.3 |
| 35. Pt-XIV.94 | 47.9 | 63.1 | 63.2 | 62.7 | 47.7 | 39.7 | 51.1 | 61.0 | 63.4 | 62.0 | | 86.1 |
| 36. Pt-II105 | 47.2 | 63.1 | 64.6 | 60.4 | 48.4 | 39.4 | 51.8 | 61.5 | 62.2 | 59.9 | 91.8 | |
| 37. Pt-123.86 | 70.9 | 48.0 | 46.6 | 48.6 | 49.7 | 47.1 | 50.7 | 46.6 | 49.0 | 50.0 | 43.9 | 42.9 |
| 38. Pt-X180 | 61.0 | 51.9 | 51.9 | 53.5 | 46.2 | 41.6 | 46.7 | 53.5 | 58.5 | 58.3 | 50.2 | 48.5 |
| 39. Pt-XII.481 | 60.0 | 50.5 | 49.5 | 46.9 | 49.5 | 41.6 | 50.0 | 50.9 | 49.5 | 49.5 | 45.1 | 42.1 |
| 40. Ck-LOB-DQ787782 | 47.2 | 72.3 | 71.4 | 68.8 | 53.4 | 44.1 | 54.7 | 71.9 | 79.4 | 78.9 | 60.6 | 62.3 |
| 41. Bn-AAP37970 | 65.2 | 52.3 | 50.8 | 47.0 | 47.7 | 44.1 | 50.0 | 50.8 | 49.6 | 51.5 | 50.4 | 49.2 |
| 42. Mt-ABE82505 | 50.3 | 70.4 | 69.1 | 67.0 | 57.4 | 46.8 | 55.1 | 71.2 | 76.9 | 78.9 | 62.7 | 62.2 |
| 43. Mt-ABE78739 | 67.2 | 50.0 | 50.0 | 50.7 | 54.7 | 43.3 | 53.2 | 48.2 | 49.6 | 48.6 | 50.0 | 48.9 |
| 44. At-LBD37-Q9FN11 | 51.4 | 66.8 | 65.6 | 64.4 | 54.2 | 47.9 | 52.9 | 67.2 | 76.0 | 74.0 | 60.8 | 60.4 |
| 45. At-LBD38-Q9SN23 | 49.7 | 65.6 | 64.4 | 60.3 | 52.3 | 44.9 | 53.6 | 66.0 | 75.3 | 72.1 | 58.7 | 61.5 |
| 46. At-LBD39-Q9SZE8 | 51.0 | 66.3 | 64.6 | 62.1 | 54.5 | 46.8 | 53.3 | 67.9 | 71.7 | 71.1 | 59.6 | 60.8 |
| 47. At-LBD40-Q9ZW96 | 61.7 | 56.7 | 55.4 | 54.9 | 49.1 | 41.0 | 49.6 | 55.8 | 55.9 | 55.4 | 50.6 | 51.5 |
| 48. At-LBD41-Q9M886 | 66.9 | 50.6 | 49.8 | 48.7 | 50.5 | 45.5 | 49.3 | 52.1 | 48.7 | 51.3 | 49.0 | 47.1 |
| 49. At-LBD42-Q9CA30 | 58.3 | 53.6 | 54.1 | 55.4 | 47.7 | 41.3 | 48.2 | 54.5 | 54.6 | 56.2 | 54.9 | 54.5 |

| | 37 | 38 | 39 | 40 | 41 | 42 | 43 | 44 | 45 | 46 |
|---|---|---|---|---|---|---|---|---|---|---|
| 1. Os-Os01g03890 | 44.0 | 39.2 | 34.8 | 35.5 | 44.4 | 34.9 | 40.5 | 31.9 | 33.3 | 32.4 |
| 2. Os-Os01g32770 | 45.3 | 38.5 | 32.3 | 32.1 | 42.7 | 33.9 | 44.8 | 30.2 | 32.1 | 29.7 |
| 3. Os-Os03g33090 | 34.7 | 36.9 | 32.7 | 53.0 | 36.4 | 54.9 | 35.1 | 50.0 | 51.2 | 49.6 |
| 4. Os-Os03g41330 | 33.7 | 35.2 | 31.2 | 43.6 | 36.2 | 44.6 | 32.6 | 43.9 | 42.4 | 40.7 |
| 5. Os-Os07g40000 | 32.8 | 35.7 | 31.9 | 49.8 | 32.6 | 51.3 | 33.1 | 49.2 | 47.4 | 48.1 |
| 6. Nb-TC9404 | 33.8 | 37.3 | 32.6 | 58.6 | 32.5 | 61.1 | 35.1 | 56.9 | 54.0 | 56.3 |
| 7. Gm-TC227562 | 32.8 | 36.4 | 32.4 | 76.6 | 34.7 | 74.9 | 37.1 | 56.2 | 57.8 | 57.3 |
| 8. Gm-TC216138 | 31.6 | 36.7 | 31.6 | 74.3 | 36.0 | 74.0 | 34.9 | 53.9 | 55.0 | 54.1 |
| 9. Hv-TC147776 | 31.1 | 38.2 | 33.5 | 50.6 | 34.3 | 50.6 | 34.5 | 49.2 | 48.6 | 45.8 |
| 10. Sb-TC104758 | 33.1 | 35.2 | 31.3 | 48.7 | 33.7 | 50.2 | 33.1 | 50.6 | 48.2 | 46.7 |
| 11. Aq-TC18561 | 33.4 | 36.2 | 32.5 | 56.7 | 36.0 | 57.8 | 36.0 | 51.6 | 55.6 | 57.1 |
| 12. Vv-TC60668 | 33.4 | 39.7 | 34.3 | 65.3 | 33.7 | 66.8 | 36.0 | 54.2 | 57.4 | 58.8 |
| 13. Lj-TC15459 | 32.1 | 35.9 | 31.0 | 70.4 | 32.8 | 70.7 | 35.3 | 52.8 | 53.0 | 54.8 |
| 14. Gh-TC30552 | 33.7 | 38.6 | 33.7 | 66.2 | 35.2 | 69.0 | 32.9 | 62.4 | 63.5 | 58.7 |
| 15. Ta-TC235711 | 45.3 | 42.7 | 36.2 | 35.4 | 46.0 | 38.3 | 46.6 | 34.1 | 35.9 | 33.7 |
| 16. St-TC133081 | 55.7 | 45.6 | 37.3 | 36.5 | 55.8 | 37.2 | 54.9 | 37.1 | 35.8 | 36.5 |
| 17. Mt-TC107091 | 63.7 | 43.0 | 37.3 | 34.2 | 54.3 | 33.9 | 60.6 | 33.4 | 37.2 | 34.2 |
| 18. Hv-TC147808 | 45.0 | 41.9 | 34.3 | 34.3 | 45.2 | 37.4 | 46.8 | 32.4 | 35.4 | 32.8 |
| 19. Vv-TC55931 | 52.0 | 51.4 | 40.9 | 36.8 | 49.8 | 38.7 | 48.9 | 37.4 | 34.9 | 36.7 |
| 20. St-TC162239 | 41.6 | 45.1 | 39.7 | 36.3 | 41.8 | 36.0 | 42.9 | 35.4 | 35.7 | 35.0 |
| 21. Pta-TC69225 | 37.6 | 33.4 | 29.4 | 33.4 | 34.7 | 32.3 | 38.0 | 31.7 | 31.1 | 32.5 |
| 22. Pta-TC67269 | 38.8 | 36.8 | 31.2 | 30.4 | 35.7 | 31.2 | 38.1 | 31.3 | 32.3 | 31.3 |
| 23. Gm-TC220806 | 48.7 | 48.8 | 40.1 | 39.7 | 48.1 | 37.9 | 51.3 | 36.5 | 38.0 | 38.4 |
| 24. Ta-TC270332 | 31.8 | 33.3 | 29.3 | 42.5 | 32.5 | 44.2 | 32.4 | 41.5 | 40.4 | 43.7 |
| 25. Aq-TC18329 | 61.1 | 48.3 | 40.6 | 33.4 | 51.2 | 36.6 | 55.9 | 35.1 | 32.8 | 34.4 |
| 26. St-TC137193 | 34.1 | 37.3 | 35.7 | 60.9 | 33.0 | 60.0 | 34.4 | 57.3 | 55.7 | 54.1 |
| 27. St-TC133385 | 34.0 | 37.3 | 36.4 | 60.1 | 34.5 | 59.6 | 33.8 | 56.5 | 54.9 | 53.1 |
| 28. St-TC140088 | 33.3 | 36.9 | 33.5 | 53.0 | 31.4 | 51.9 | 31.9 | 48.8 | 47.0 | 49.6 |
| 29. Pa-TC14656 | 32.1 | 33.3 | 33.2 | 41.1 | 29.3 | 41.7 | 32.8 | 39.0 | 40.0 | 39.9 |
| 30. Pta-TC59178 | 34.0 | 30.3 | 28.3 | 37.4 | 32.8 | 38.4 | 30.2 | 36.5 | 35.7 | 37.6 |
| 31. Pta-TC67974 | 33.2 | 34.2 | 34.1 | 42.2 | 31.9 | 41.9 | 34.2 | 40.9 | 42.6 | 40.4 |
| 32. Le-TC178827 | 32.8 | 38.9 | 36.5 | 60.8 | 34.1 | 60.7 | 33.1 | 56.5 | 55.5 | 55.3 |
| 33. Pt-III.589 | 34.7 | 37.9 | 33.5 | 67.1 | 35.5 | 65.7 | 32.7 | 64.1 | 63.9 | 57.5 |
| 34. Pt-V.543 | 34.3 | 36.6 | 33.3 | 67.4 | 34.5 | 66.7 | 32.0 | 62.6 | 62.9 | 59.3 |
| 35. Pt-XIV.94 | 31.8 | 35.8 | 30.4 | 49.4 | 36.0 | 51.7 | 37.1 | 45.0 | 45.3 | 46.5 |
| 36. Pt-II105 | 32.4 | 35.0 | 30.4 | 48.5 | 36.7 | 51.1 | 35.6 | 43.6 | 45.7 | 48.1 |
| 37. Pt-123.86 | | 45.8 | 39.3 | 33.1 | 56.2 | 34.3 | 55.9 | 34.8 | 34.1 | 33.0 |
| 38. Pt-X180 | 56.4 | | 43.1 | 34.6 | 42.3 | 34.9 | 44.1 | 37.2 | 37.3 | 39.9 |
| 39. Pt-XII.481 | 55.4 | 56.8 | | 32.5 | 37.5 | 33.6 | 37.4 | 31.9 | 30.5 | 29.9 |
| 40. Ck-LOB-DQ787782 | 49.0 | 53.9 | 47.3 | | 35.5 | 80.9 | 33.8 | 54.0 | 55.8 | 57.8 |
| 41. Bn-AAP37970 | 70.3 | 56.8 | 55.7 | 51.5 | | 37.7 | 51.6 | 33.2 | 34.1 | 34.3 |
| 42. Mt-ABE82505 | 47.0 | 55.2 | 47.3 | 87.1 | 51.9 | | 36.7 | 55.6 | 57.3 | 56.7 |
| 43. Mt-ABE78739 | 67.6 | 57.9 | 55.4 | 50.7 | 64.7 | 51.4 | | 31.9 | 32.7 | 34.3 |
| 44. At-LBD37-Q9FN11 | 53.0 | 53.2 | 48.0 | 69.2 | 50.4 | 66.8 | 50.0 | | 76.9 | 55.3 |
| 45. At-LBD38-Q9SN23 | 47.0 | 55.9 | 49.5 | 70.0 | 51.5 | 69.2 | 48.6 | 86.4 | | 56.8 |
| 46. At-LBD39-Q9SZE8 | 49.0 | 57.3 | 49.1 | 72.5 | 53.8 | 71.3 | 51.1 | 67.2 | 68.8 | |
| 47. At-LBD40-Q9ZW96 | 65.5 | 62.7 | 52.0 | 55.8 | 68.9 | 56.2 | 66.2 | 55.2 | 55.5 | 56.3 |
| 48. At-LBD41-Q9M886 | 70.9 | 58.6 | 57.1 | 47.9 | 90.5 | 50.2 | 65.5 | 53.2 | 51.7 | 52.1 |
| 49. At-LBD42-Q9CA30 | 55.4 | 62.7 | 52.0 | 58.4 | 56.8 | 55.8 | 57.2 | 53.6 | 52.6 | 52.9 |

| | 47 | 48 | 49 |
|---|---|---|---|
| 1. Os-Os01g03890 | 41.5 | 41.3 | 38.2 |
| 2. Os-Os01g32770 | 41.5 | 44.8 | 39.0 |
| 3. Os-Os03g33090 | 40.4 | 35.0 | 40.8 |
| 4. Os-Os03g41330 | 38.2 | 33.7 | 36.0 |
| 5. Os-Os07g40000 | 40.2 | 33.8 | 36.3 |
| 6. Nb-TC9404 | 39.4 | 34.6 | 40.7 |
| 7. Gm-TC227562 | 40.8 | 35.7 | 39.8 |
| 8. Gm-TC216138 | 36.5 | 34.0 | 35.5 |
| 9. Hv-TC147776 | 38.2 | 34.6 | 37.7 |
| 10. Sb-TC104758 | 36.2 | 34.5 | 38.6 |
| 11. Aq-TC18561 | 42.1 | 37.2 | 36.6 |
| 12. Vv-TC60668 | 40.2 | 36.1 | 38.2 |
| 13. Lj-TC15459 | 38.1 | 35.1 | 36.4 |
| 14. Gh-TC30552 | 39.8 | 34.6 | 36.6 |
| 15. Ta-TC235711 | 44.3 | 44.3 | 41.0 |
| 16. St-TC133081 | 50.7 | 55.9 | 45.7 |
| 17. Mt-TC107091 | 52.6 | 56.1 | 40.2 |
| 18. Hv-TC147808 | 42.1 | 46.5 | 39.7 |
| 19. Vv-TC55931 | 49.2 | 49.3 | 47.3 |
| 20. St-TC162239 | 46.5 | 44.5 | 47.5 |
| 21. Pta-TC69225 | 36.4 | 36.2 | 34.1 |
| 22. Pta-TC67269 | 36.0 | 36.3 | 33.3 |
| 23. Gm-TC220806 | 52.8 | 50.8 | 49.8 |
| 24. Ta-TC270332 | 39.2 | 30.4 | 36.3 |
| 25. Aq-TC18329 | 49.8 | 53.2 | 43.7 |
| 26. St-TC137193 | 39.2 | 34.2 | 38.6 |
| 27. St-TC133385 | 40.1 | 34.1 | 38.4 |
| 28. St-TC140088 | 37.8 | 35.7 | 36.6 |
| 29. Pa-TC14656 | 33.8 | 30.4 | 31.3 |
| 30. Pta-TC59178 | 30.2 | 32.5 | 27.2 |
| 31. Pta-TC67974 | 34.8 | 31.8 | 33.0 |
| 32. Le-TC178827 | 39.0 | 34.6 | 38.5 |
| 33. Pt-III.589 | 36.6 | 35.0 | 35.3 |
| 34. Pt-V.543 | 38.2 | 35.4 | 39.2 |
| 35. Pt-XIV.94 | 37.8 | 33.8 | 38.3 |
| 36. Pt-II105 | 40.2 | 35.7 | 37.4 |
| 37. Pt-123.86 | 53.8 | 59.1 | 43.6 |
| 38. Pt-X180 | 46.8 | 41.5 | 49.6 |
| 39. Pt-XII.481 | 38.5 | 37.6 | 36.4 |
| 40. Ck-LOB-DQ787782 | 40.2 | 31.6 | 38.2 |
| 41. Bn-AAP37970 | 55.5 | 83.5 | 42.1 |
| 42. Mt-ABE82505 | 40.3 | 33.8 | 36.8 |
| 43. Mt-ABE78739 | 53.3 | 51.6 | 43.8 |
| 44. At-LBD37-Q9FN11 | 37.5 | 34.5 | 34.9 |
| 45. At-LBD38-Q9SN23 | 40.5 | 33.5 | 34.8 |
| 46. At-LBD39-Q9SZE8 | 38.3 | 35.0 | 37.1 |
| 47. At-LBD40-Q9ZW96 | | 56.7 | 46.5 |
| 48. At-LBD41-Q9M886 | 68.4 | | 42.6 |
| 49. At-LBD42-Q9CA30 | 61.8 | 56.3 | |

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 2 are presented in Table A3.

TABLE A3
InterPro scan results (major accession numbers) of the
polypeptide sequence as represented by SEQ ID NO: 2.

| | | | Amino acid |
|---|---|---|---|
| | Accession | Accession | coordinates on |
| Database | number | name | SEQ ID NO 2 |
| PFAM | PF03195 | DUF260 | 2-107 |
| PROFILE | PS50891 | LOB | 1-107 |

### Example 5

### Topology Prediction of the Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 2 are presented Table A4. The “plant” organism group has been selected, no cutoffs defined, and the predicted length of the transit peptide requested,

TABLE A4
TargetP 1.1 analysis of the polypeptide sequence as
represented by SEQ ID NO: 2

| | |
|---|---|
| Length (AA) | 250 |
| Chloroplastic transit peptide | 0.026 |
| Mitochondrial transit peptide | 0.401 |
| Secretory pathway signal peptide | 0.019 |
| Other subcellular targeting | 0.573 |
| Predicted Location | / |
| Reliability class | 5 |
| Predicted transit peptide length | / |

The subcellular localization of the polypeptide sequence as represented by SEQ ID NO: 2 may be the cytoplasm or nucleus, no transit peptide is predicted. SubLoc (Hua & Sun, Bioinformatics 17, 721-728, 2001) predicts a nuclear localisation (reliability index: 2, accuracy: 74%); this prediction is in agreement with the data from Liu et al (2005).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 6

### Cloning of the LSD Nucleic Acid Sequence Used in the Methods of the Invention

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm009067 (SEQ ID NO: 3; sense, start codon in bold):

5′-ggggacaagtttgtacaaaaaagcaggcttaaacaatgagctgc
aatggttgc-3′

and prm009068 (SEQ ID NO: 4; reverse, complementary):

5′-ggggaccactttgtacaagaaagctgggtactaactctgagaaaa
ccgcc-3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pLBD. Plasmid pDONR201 was purchased from invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 10) for root specific expression was located upstream of this Gateway cassette.

After the LR recombination step, the resulting expression vector pGOS2::LBD (FIG. 4) was transformed into Agrobacterium strain LBA4044 according to methods well known in the art.

### Example 7

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were subcultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K₂SO₄, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton (Gossypium hirsutum L) transformation is performed using Agrobacterium tumefaciens, on hypocotyls explants. The commercial cultivars such as Coker 130 or Coker 312 (SeedCo, Lubbock, Tex.) are standard varieties used for transformation, but other varieties can also be used. The seeds are surface sterilized and germinated in the dark. Hypocotyl explants are cut from the germinated seedlings to lengths of about 1-1.5 centimeter. The hypotocyl explant is submersed in the Agrobacterium tumefaciens inoculum containing the expression vector, for 5 minutes then co-cultivated for about 48 hours on MS+1.8 mg/l KNO3+2% glucose at 24° C., in the dark. The explants are transferred the same medium containing appropriate bacterial and plant selectable markers (renewed several times), until embryogenic calli is seen. The calli are separated and subcultured until somatic embryos appear. Plantlets derived from the somatic embryos are matured on rooting medium until roots develop. The rooted shoots are transplanted to potting soil in the greenhouse. T1 seeds are produced from plants that exhibit tolerance to the selection agent and that contain a single copy of the T-DNA insert.

### Example 8

### Phenotypic Evaluation Procedure

### 8.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are supplied with water at regular intervals to ensure that water and nutrients are not limiting to satisfy plant needs to complete growth and development.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### 8.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

### 8.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The seed fill rate as defined in the present invention is the proportion (expressed as a %) of the number of filled seeds over the total number of seeds (or florets).

### Example 9

### Results of the Phenotypic Evaluation of the Transgenic Plants Comprising SEQ ID NO: 1

The evaluation of transgenic rice plants expressing a LBD nucleic acid under non-stress conditions showed that there was an increase of more than 5% for aboveground biomass (AreaMax), total seed yield, number of filled seeds, fill rate, harvest index, and more than 3% for thousand kernel weight.

The evaluation of transgenic rice plants expressing a LBD nucleic acid in the nitrogen use efficiency screen revealed an increase of more than 5% for emergence vigour (early vigour).

### Example 10

### Results of the Phenotypic Evaluation of the Transgenic Plants Comprising SEQ ID NO: 71

The coding region comprised in SEQ ID NO: 71 was cloned under the control of the rice GOS2 promoter into a rice transformation vector as described in Example 6. Transgenic rice plants comprising the coding region of SEQ ID NO: 71 were generated following the procedures of Example 7. Plants were evaluated according to the procedure described in Example 8. SEQ ID NO: 71 encodes the LBD protein represented by SEQ ID NO: 25.

The evaluation of transgenic rice plants expressing SEQ ID NO: 71 under non-stress conditions showed that there was an increase of more than 5% for aboveground biomass (AreaMax), the number of flowers per panicle, the total number of seeds per plant, and more than 3% for thousand kernel weight.

### Example 11

### Results of the Phenotypic Evaluation of the Transgenic Plants Comprising SEQ ID NO: 72

The coding region comprised in SEQ ID NO: 72 was cloned under the control of the rice GOS2 promoter into a rice transformation vector as described in Example 6. Transgenic rice plants comprising the coding region of SEQ ID NO: 72 were generated following the procedures of Example 7. Plants were evaluated according to the procedure described in Example 8. SEQ ID NO: 72 encodes the LBD protein represented by SEQ ID NO: 52.

The evaluation of transgenic rice plants expressing SEQ ID NO: 72 under non-stress conditions showed that there was an increase of more than 5% for aboveground biomass (AreaMax), seed weight per plant, number of filled seeds, the number of flowers per panicle, the total number of seeds per plant, harvest index and more than 3% for thousand kernel weight.

The evaluation of transgenic rice plants expressing SEQ ID NO: 72 in the nitrogen use efficiency screen revealed an increase of more than 5% for the aboveground biomass (AreaMax).

### II. JMJC (JUMONJI-C) Polypeptide

### Example 12

### Identification of Sequences Related to the JMJC Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCB)) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length.

Table 81 provides a list of nucleic acid sequences related to the JMJC nucleic acid sequence used in the methods of the present invention. Polypeptides with an accession extended by a dot and one digit represent splice variants.

TABLE B1
Examples of JMJC polypeptides

| | Genbank | | Protein |
|---|---|---|---|
| | accession | Nucleic acid | SEQ ID |
| Plant Source | (or locus) number | SEQ ID NO: | NO: |
| Arabidopsis thaliana | AT3G20810 | 73 | 74 |
| Arabidopsis thaliana | AT3G20810 | 83 | 84 |
| Arabidopsis thaliana | AT5G19840 | 85 | 86 |
| Arabidopsis thaliana | AT3G45880 | 87 | 88 |
| Medicago truncatula | ABE92082 | 89 | 90 |
| Brachypodium sylvaticum | CAJ26373 | 91 | 92 |
| Oryza sativa | Os09g0483600 | 93 | 94 |
| Arabidopsis thaliana | AT1G08620 | 95 | 96 |
| Arabidopsis thaliana | AT1G09060 | 97 | 98 |
| Arabidopsis thaliana | AT1G09060 | 99 | 100 |
| Arabidopsis thaliana | AT1G09060 | 101 | 102 |
| Arabidopsis thaliana | AT1G11950 | 103 | 104 |
| Arabidopsis thaliana | AT1G30810 | 105 | 106 |
| Arabidopsis thaliana | AT1G62310 | 107 | 108 |
| Arabidopsis thaliana | AT1G63490 | 109 | 110 |
| Arabidopsis thaliana | AT1G78280 | 111 | 112 |
| Arabidopsis thaliana | AT2G34880 | 113 | 114 |
| Arabidopsis thaliana | AT2G38950 | 115 | 116 |
| Arabidopsis thaliana | AT3G07610 | 117 | 118 |
| Arabidopsis thaliana | AT3G48430 | 119 | 120 |
| Arabidopsis thaliana | AT4G00990 | 121 | 122 |
| Arabidopsis thaliana | AT4G20400 | 123 | 124 |
| Arabidopsis thaliana | AT4G20400 | 125 | 126 |
| Arabidopsis thaliana | AT5G04240 | 127 | 128 |
| Arabidopsis thaliana | AT5G06550 | 129 | 130 |
| Arabidopsis thaliana | AT5G46910 | 131 | 132 |
| Arabidopsis thaliana | AT5G63080 | 133 | 134 |
| Oryza sativa | Os01g36630 | 135 | 136 |
| Oryza sativa | Os01g67970 | 137 | 138 |
| Oryza sativa | Os02g01940 | 139 | 140 |
| Oryza sativa | Os02g58210 | 141 | 142 |
| Oryza sativa | Os02g58210 | 143 | 144 |
| Oryza sativa | Os03g05680 | 145 | 146 |
| Oryza sativa | Os03g22540 | 147 | 148 |
| Oryza sativa | Os03g27250 | 149 | 150 |
| Oryza sativa | Os03g31594 | 151 | 152 |
| Oryza sativa | Os03g31594 | 153 | 154 |
| Oryza sativa | Os05g10770 | 155 | 156 |
| Oryza sativa | Os05g23670 | 157 | 158 |
| Oryza sativa | Os09g22540 | 159 | 160 |
| Oryza sativa | Os10g42690 | 161 | 162 |
| Oryza sativa | Os11g36450 | 163 | 164 |
| Oryza sativa | Os12g18149 | 165 | 166 |
| Oryza sativa | Os12g18150 | 167 | 168 |
| Glycine max | Gm_JMJ_1 | 169 | 170 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### Example 13

### Alignment of JMJC Polypeptide Sequences

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0,1 and the selected weight matrix is Blosum 62 (if polypeptides are aligned). Minor manual editing may be done to further optimise the alignment. Sequence conservation among JMJC polypeptides is mostly in the JmjC domain of the polypeptides. The region corresponding to the motifs represented by SEQ ID NO: 79 and by SEQ ID NO: 81 is more conserved than that of motif 8. A consensus sequence is given. Amino acid residues in the consensus sequences are highly conserved. Blanks in the conserved sequences represent any amino acid. The JMJC polypeptides are aligned in FIG. 7.

### Example 14

### Calculation of Global Percentage Identity Between JMJC Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B2 for the global similarity and identity over the full length of the polypeptide sequences. Percentage identity is given above the diagonal in bold and percentage similarity is given below the diagonal (normal face).

The percentage identity between the JMJC polypeptide sequences useful in performing the methods of the invention can be as low as 15% amino acid identity compared to SEQ ID NO: 74.

TABLE B2
MatGAT results for global similarity and identity over the full length of the
polypeptide sequences.

| | | SEQ ID | SEQ ID | SEQ ID | SEQ ID | SEQ ID | SEQ ID | SEQ ID |
|---|---|---|---|---|---|---|---|---|
| | | NO: 74 | NO: 84 | NO: 86 | NO: 90 | NO: 92 | NO: 94 | NO: 136 |
| AT3G20810.1 | SEQ ID NO: 74 | | 97.4 | 16.1 | 15.5 | 16.9 | 17.3 | 15.5 |
| AT3G20810.2 | SEQ ID NO: 84 | 97.4 | | 15.5 | 14.5 | 17.5 | 17.1 | 14.9 |
| AT5G19840 | SEQ ID NO: 86 | 30.3 | 30.7 | | 16.9 | 17 | 16.9 | 16.9 |
| ABE92082 | SEQ ID NO: 90 | 28.2 | 27.7 | 28.5 | | 51.8 | 51.7 | 17.3 |
| CAJ26373 | SEQ ID NO: 92 | 30.1 | 29.6 | 27.7 | 67.6 | | 82.5 | 12.4 |
| Os09g0483600 | SEQ ID NO: 94 | 30.1 | 30.1 | 27.5 | 67.3 | 89.6 | | 16.1 |
| Os01g36630 | SEQ ID NO: 136 | 33.7 | 31.2 | 32.9 | 34.2 | 29.1 | 29.6 | |

### Example 15

### Identification of Domains Comprised in JMJC Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

Table B3 shows the settings (Gathering cut off, trusted cut off and noise cut off) as described in the Pfam database that were used to produce the HMMs_fs for the different domains.

TABLE B3
HMMs_fs settings.

| Domain | Gathering cutoff | Trusted cutoff | Noise cutoff |
|---|---|---|---|
| PF02373 | 16 | 16.1 | 15.9 |
| PF02375 | 15 | 16.8 | 13.8 |
| PF02928 | 25 | 44.4 | 21.1 |
| PF00646 | 17.7 | 17.7 | 17.6 |
| PF00096 | 22.5 | 22.5 | 22.4 |
| PF04967 | 15.4 | 15.4 | 3 |

The results of the Pfam scan for representative AUG polypeptides of plant origin are presented in Table B4. The amino acid coordinates for each domain in the sequence of reference is indicated in the columns Start and End. The E-value of the alignment is also given. The InterPro ID accession number of each domain identified is also provided.

TABLE B4
Pfam scan results (major accession numbers) of representative JMJC polypeptides
of plant origin.

| | Genbank (genomic | | | | | | | |
|---|---|---|---|---|---|---|---|---|
| Protein | locus) accession | | | | | | | InterPro |
| SEQ ID NO: | number (name) | Database | Entry | E_value | Start | End | InterPro ID | Description |
| 96 | AT1G08620 | Pfam | PF02373 | 1.10E−62 | 368 | 484 | IPR003347 | Transcription |
| | | | | | | | | factor jumonji, |
| | | | | | | | | jmjC |
| 96 | AT1G08620 | Pfam | PF02375 | 1.50E−16 | 127 | 165 | IPR003349 | Transcription |
| | | | | | | | | factor jumonji, |
| | | | | | | | | JmjN |
| 96 | AT1G08620 | Pfam | PF02928 | 9.10E−22 | 591 | 644 | IPR004198 | Zn-finger, |
| | | | | | | | | C5HC2 type |
| 96 | AT1G08620 | SMART | SM00541 | 1.10E−17 | 962 | 1006 | IPR003888 | FY-rich domain, |
| | | | | | | | | N-terminal |
| 96 | AT1G08620 | SMART | SM00542 | 1.30E−43 | 1012 | 1106 | IPR003889 | FY-rich domain, |
| | | | | | | | | C-terminal |
| 98 | AT1G09060 | Pfam | PF02373 | 6.80E−06 | 644 | 856 | IPR003347 | Transcription |
| | | | | | | | | factor jumonji, |
| | | | | | | | | jmjC |
| 104 | AT1G11950 | Pfam | PF02373 | 1.00E−23 | 645 | 825 | IPR003347 | Transcription |
| | | | | | | | | factor jumonji, |
| | | | | | | | | jmjC |
| 106 | AT1G30810 | Pfam | PF02373 | 5.00E−30 | 294 | 378 | IPR003347 | Transcription |
| | | | | | | | | factor jumonji, |
| | | | | | | | | jmjC |
| 106 | AT1G30810 | Pfam | PF02375 | 3.10E−24 | 58 | 104 | IPR003349 | Transcription |
| | | | | | | | | factor jumonji, |
| | | | | | | | | JmjN |
| 106 | AT1G30810 | Pfam | PF02928 | 4.50E−30 | 487 | 540 | IPR004198 | Zn-finger, |
| | | | | | | | | C5HC2 type |
| 106 | AT1G30810 | SMART | SM00541 | 1.30E−17 | 626 | 670 | IPR003888 | FY-rich domain, |
| | | | | | | | | N-terminal |
| 106 | AT1G30810 | SMART | SM00542 | 1.20E−39 | 676 | 762 | IPR003889 | FY-rich domain, |
| | | | | | | | | C-terminal |
| 108 | AT1G62310 | Pfam | PF02373 | 2.00E−27 | 733 | 846 | IPR003347 | Transcription |
| | | | | | | | | factor jumonji, |
| | | | | | | | | jmjC |
| 108 | AT1G62310 | ProSite | PS50089 | 10.173 | 209 | 256 | IPR001841 | Zn-finger, RING |
| 110 | AT1G63490 | Pfam | PF00628 | 5.40E−08 | 1009 | 1053 | IPR001965 | Zn-finger-like, |
| | | | | | | | | PHD finger |
| 110 | AT1G63490 | Pfam | PF02373 | 3.10E−60 | 66 | 182 | IPR003347 | Transcription |
| | | | | | | | | factor jumonji, |
| | | | | | | | | jmjC |
| 110 | AT1G63490 | Pfam | PF02928 | 4.80E−33 | 276 | 329 | IPR004198 | Zn-finger, |
| | | | | | | | | C5HC2 type |
| 112 | AT1G78280 | Pfam | PF00646 | 0.00012 | 15 | 62 | IPR001810 | Cyclin-like F- |
| | | | | | | | | box |
| 112 | AT1G78280 | Pfam | PF02373 | 4.10E−12 | 249 | 362 | IPR003347 | Transcription |
| | | | | | | | | factor jumonji, |
| | | | | | | | | jmjC |
| 112 | AT1G78280 | ProSite | PS50181 | 9.603 | 14 | 60 | IPR001810 | Cyclin-like F- |
| | | | | | | | | box |
| 114 | AT2G34880 | Pfam | PF02373 | 1.50E−67 | 294 | 410 | IPR003347 | Transcription |
| | | | | | | | | factor jumonji, |
| | | | | | | | | jmjC |
| 114 | AT2G34880 | Pfam | PF02375 | 3.70E−29 | 60 | 106 | IPR003349 | Transcription |
| | | | | | | | | factor jumonji, |
| | | | | | | | | JmjN |
| 114 | AT2G34880 | Pfam | PF02928 | 1.40E−23 | 514 | 567 | IPR004198 | Zn-finger, |
| | | | | | | | | C5HC2 type |
| 114 | AT2G34880 | SMART | SM00541 | 9.90E−17 | 643 | 687 | IPR003888 | FY-rich domain, |
| | | | | | | | | N-terminal |
| 114 | AT2G34880 | SMART | SM00542 | 4.60E−39 | 693 | 781 | IPR003889 | FY-rich domain, |
| | | | | | | | | C-terminal |
| 116 | AT2G38950 | Pfam | PF02373 | 4.60E−47 | 321 | 437 | IPR003347 | Transcription |
| | | | | | | | | factor jumonji, |
| | | | | | | | | jmjC |
| 116 | AT2G38950 | Pfam | PF02375 | 9.30E−28 | 107 | 153 | IPR003349 | Transcription |
| | | | | | | | | factor jumonji, |
| | | | | | | | | JmjN |
| 116 | AT2G38950 | Pfam | PF02928 | 5.90E−27 | 544 | 597 | IPR004198 | Zn-finger, |
| | | | | | | | | C5HC2 type |
| 118 | AT3G07610 | Pfam | PF02373 | 3.40E−15 | 726 | 843 | IPR003347 | Transcription |
| | | | | | | | | factor jumonji, |
| | | | | | | | | jmjC |
| 120 | AT3G48430 | Pfam | PF00096 | 0.014 | 1243 | 1268 | IPR007087 | Zn-finger, C2H2 |
| | | | | | | | | type |
| 120 | AT3G48430 | Pfam | PF00096 | 0.0022 | 1296 | 1320 | IPR007087 | Zn-finger, C2H2 |
| | | | | | | | | type |
| 120 | AT3G48430 | Pfam | PF00096 | 0.00026 | 1326 | 1352 | IPR007087 | Zn-finger, C2H2 |
| | | | | | | | | type |
| 120 | AT3G48430 | Pfam | PF02373 | 9.00E−52 | 233 | 352 | IPR003347 | Transcription |
| | | | | | | | | factor jumonji, |
| | | | | | | | | jmjC |
| 120 | AT3G48430 | Pfam | PF02375 | 1.30E−07 | 19 | 62 | IPR003349 | Transcription |
| | | | | | | | | factor jumonji, |
| | | | | | | | | JmjN |
| 120 | AT3G48430 | ProSite | PS00028 | 0.00008 | 1268 | 1290 | IPR007087 | Zn-finger, C2H2 |
| | | | | | | | | type |
| 122 | AT4G00990 | Pfam | PF02373 | 2.80E−16 | 607 | 781 | IPR003347 | Transcription |
| | | | | | | | | factor jumonji, |
| | | | | | | | | jmjC |
| 124 | AT4G20400 | Pfam | PF02373 | 4.60E−68 | 239 | 355 | IPR003347 | Transcription |
| | | | | | | | | factor jumonji, |
| | | | | | | | | jmjC |
| 124 | AT4G20400 | Pfam | PF02375 | 9.60E−11 | 7 | 44 | IPR003349 | Transcription |
| | | | | | | | | factor jumonji, |
| | | | | | | | | JmjN |
| 124 | AT4G20400 | Pfam | PF02928 | 5.70E−30 | 462 | 515 | IPR004198 | Zn-finger, |
| | | | | | | | | C5HC2 type |
| 124 | AT4G20400 | SMART | SM00541 | 4.40E−15 | 683 | 727 | IPR003888 | FY-rich domain, |
| | | | | | | | | N-terminal |
| 124 | AT4G20400 | SMART | SM00542 | 7.40E−41 | 733 | 830 | IPR003889 | FY-rich domain, |
| | | | | | | | | C-terminal |
| 128 | AT5G04240 | Pfam | PF00096 | 0.41 | 1228 | 1253 | IPR007087 | Zn-finger, C2H2 |
| | | | | | | | | type |
| 128 | AT5G04240 | Pfam | PF00096 | 0.0028 | 1281 | 1305 | IPR007087 | Zn-finger, C2H2 |
| | | | | | | | | type |
| 128 | AT5G04240 | Pfam | PF00096 | 0.0016 | 1311 | 1337 | IPR007087 | Zn-finger, C2H2 |
| | | | | | | | | type |
| 128 | AT5G04240 | Pfam | PF02373 | 1.10E−48 | 292 | 411 | IPR003347 | Transcription |
| | | | | | | | | factor jumonji, |
| | | | | | | | | jmjC |
| 128 | AT5G04240 | Pfam | PF02375 | 1.00E−09 | 15 | 58 | IPR003349 | Transcription |
| | | | | | | | | factor jumonji, |
| | | | | | | | | JmjN |
| 130 | AT5G06550 | Pfam | PF00646 | 0.0013 | 81 | 128 | IPR001810 | Cyclin-like F- |
| | | | | | | | | box |
| 130 | AT5G06550 | Pfam | PF02373 | 3.20E−07 | 311 | 422 | IPR003347 | Transcription |
| | | | | | | | | factor jumonji, |
| | | | | | | | | jmjC |
| 130 | AT5G46910 | Pfam | PF02373 | 1.00E−49 | 199 | 322 | IPR003347 | Transcription |
| | | | | | | | | factor jumonji, |
| | | | | | | | | jmjC |
| 132 | AT5G46910 | Pfam | PF02375 | 2.90E−09 | 21 | 62 | IPR003349 | Transcription |
| | | | | | | | | factor jumonji, |
| | | | | | | | | JmjN |
| 134 | AT5G63080 | Pfam | PF02373 | 3.90E−16 | 164 | 270 | IPR003347 | Transcription |
| | | | | | | | | factor jumonji, |
| | | | | | | | | jmjC |
| 74 | AT3G20810 | Pfam | PF02373 | 0.00013 | 378 | 409 | IPR003347 | Transcription |
| | | | | | | | | factor jumonji, |
| | | | | | | | | jmjC |
| 84 | AT5G19840 | Pfam | PF02373 | 0.036 | 173 | 281 | IPR003347 | Transcription |
| | | | | | | | | factor jumonji, |
| | | | | | | | | jmjC |
| 86 | AT5G19840 | Pfam | PF04967 | 0.47 | 295 | 307 | IPR007050 | HTH DNA |
| | | | | | | | | binding domain |
| 170 | Gm_JMJ_1 | Pfam | PF02373.11 | 9.70E−06 | 297 | 404 | IPR003347 | JmjC domain |

### Example 16

### Cloning of the JMJC Nucleic Acid Sequence Used in the Methods of the Invention

The nucleic acid sequence used in the methods of the invention was amplified by PCR using as template a custom-made Arabidopsis thaliana seedlings cDNA library (in pGMV Sport 6.0; Invitrogen, Paisley, UK). PCR was performed using Hifi Taq DNA polymerase in standard conditions, using 200 ng of template in a 50 μl PCR mix. An upstream and downstream primer as represented by SEQ ID NO: 75 and SEQ ID NO: 76 respectively were used to amplify by PCR (Polymerase Chain Reaction) the coding region of JMJC as represented by SEQ ID NO: 73. The primers include the AttB sites for Gateway recombination to facilitate the cloning of the amplified PCR DNA fragment into a Gateway cloning vector.

The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pJMJC. Plasmid pDONR201 was purchased from Invitrogen, as part of the Gateway® technology.

The entry clone comprising SEQ ID NO: 73 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 77) for constitutive expression was located upstream of this Gateway cassette.

After the LR recombination step, the resulting expression vector pGOS2::JMJC (FIG. 9) was transformed into Agrobacterium strain LBA4044 according to methods well known in the art.

### Example 17

### Plant Transformation

Transformation of plants was carried out according to the procedure outlined in Example 7

### Example 18

### Phenotypic Evaluation Procedure

### 18.1 Evaluation Setup

Approximately 35 independent T0 rice transformants were generated. The primary transformants were transferred from a tissue culture chamber to a greenhouse for growing and harvest of T1 seed. Eight events: of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are supplied with water at regular intervals to ensure that water and nutrients are not limiting to satisfy plant needs to complete growth and development.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### 18.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P values were obtained by comparing likelihood ratio test to chi square distributions.

### 18.3 Parameters Measured

### Biomass-Related Parameter Measurement

From the stage of sowing until the stage of maturity the plants were passed several times through a digital imaging cabinet. At each time digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁸. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 19

### Results of the Phenotypic Evaluation of the Transgenic Plants

The results of the evaluation of transgenic rice plants expressing the JMJC nucleic acid as represented by SEQ ID NO: 73 under non-stress conditions are presented below. An increase of at least 5% was observed for emergence vigour (early vigour), root/shoot index, total seed yield, harvest index, and of at least 3% for thousand kernel weight in the transgenic plants of when compared to the control nullizygote plants.

The results of the evaluation of transgenic rice plants expressing SEQ ID NO: 73 under drought-stress conditions are presented hereunder. An increase of at least 5% was observed for root/shoot index, total seed weight, number of filled seeds, fill rate, harvest index and of at least 3% for thousand kernel weight in the transgenic plants of when compared to the control nullizygote plants.

### III. Casein Kinase I

### Example 20

### Identification of Sequences Related to the CKI Nucleic Acid Sequence Used in the Methods of the Invention

Sequences (full length cDNA, ESTs or genomic) related to the nucleic acid sequence used in the methods of the present invention are identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acids used in the present invention are used for the TBLASTN algorithm, with default settings and the filter to ignore low complexity sequences set off. The output of the analysis is viewed by pairwise comparison, and ranked according to the probability score (E-value), where the score reflects the probability that a particular alignment occurs by chance (the lower the E-value, the more significant the hit). In addition to E-values, comparisons are also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

In some instances, related sequences may tentatively be assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### Example 21

### Alignment of GRP Polypeptide Sequences

Alignment of polypeptide sequences is performed using the AlignX programme from the Vector NTI package (Invitrogen) which is based on the popular Clustal W algorithm of progressive alignment (Thompson et al. (1997) Nucleic Acids Res 25:4876-4882; Chema at al. (2003). Nucleic Acids Res 31:3497-3500). Default values are for the gap open penalty of 10, for the gap extension penalty of 0,1 and the selected weight matrix is Blosum 62 (if polypeptides are aligned). Minor manual editing may be done to further optimise the alignment.

A phylogenetic tree of GRP polypeptides is constructed using a neighbour-joining clustering algorithm as provided in the AlignX programme from Vector NTI (Invitrogen).

### Example 22

### Calculation of Global Percentage Identity Between Polypeptide Sequences Useful in Performing the Methods of the Invention

Global percentages of similarity and identity between full length polypeptide sequences useful in performing the methods of the invention are determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

A MATGAT table for local alignment of a specific domain, or data on % identity/similarity between specific domains may also be generated.

### Example 23

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The protein sequences representing the GRP are used as query to search the InterPro database.

### Example 24

### Topology Prediction of the Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The protein sequences representing the GRP are used to query TargetP 1.1. The “plant” organism group is selected, no cutoffs defined, and the predicted length of the transit peptide requested.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 25

### Cloning of the Nucleic Acid Sequence Used in the Methods of the Invention

### Cloning of SEQ ID NO: 171:

The nucleic acid sequence SEQ ID NO: 171 used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm8667 (SEQ ID NO: 177; sense, start codon in bold):

5′-ggggacaagtttgtacaaaaaagcaggcttaaacaatggactgc
aacatggtatct-3′

and prm8668 (SEQ ID NO: 178; reverse, complementary):

5′-ggggaccactttgtacaagaaagctgggtcacattacttactcatc
tattttgg-3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

### Cloning of SEQ ID NO: 173:

A cDNA-AFLP experiment was performed on a synchronized tobacco BY2 cell culture (Nicotiana tabacum L. cv. Bright Yellow-2), and BY2 expressed sequence tags that were cell cycle modulated were elected for further cloning. The expressed sequence tags were used to screen a tobacco cDNA library and to isolate the full-length cDNA of interest, namely one coding for SEQ ID NO: 173.

A tobacco BY2 (Nicotiana tabacum L. cv. Bright Yellow-2) cultured cell suspension was synchronized by blocking cells in early S-phase with aphidicolin as follows. The cell suspension of Nicotiana tabacum L. cv. Bright Yellow 2 was maintained as described (Nagata et al. Int. Rev. Cytol. 132, 1-30, 1992). For synchronization, a 7-day-old stationary culture was diluted 10-fold in fresh medium supplemented with aphidicolin (Sigma-Aldrich, St. Louis, Mo.; 5 mg/l). After 24 h, cells were released from the block by several washings with fresh medium after which their cell cycle progression resumed.

Total RNA was prepared using LiCl precipitation and poly(A⁺) RNA was extracted from 500 μg of total RNA using Oligotex columns (Qiagen, Hilden, Germany) according to the manufacturer's instructions. Starting from 1 μg of poly(A⁺) RNA, first-strand cDNA was synthesized by reverse transcription with a biotinylated oligo-dT₂₅ primer (Genset, Paris, France) and Superscript II (Life Technologies, Gaithersburg, Md.). Second-strand synthesis was done by strand displacement with Escherichia coli ligase (Life Technologies), DNA polymerase I (USB, Cleveland, Ohio) and RNAse-H (USB).

Five hundred ng of double-stranded cDNA was used for AFLP analysis as described (Vos et al., Nucleic Acids Res. 23 (21) 4407-4414, 1995; Bachem et al., Plant J. 9 (5) 745-53, 1996) with modifications. The restriction enzymes used were BstYI and MseI (Biolabs) and the digestion was done in two separate steps. After the first restriction digest with one of the enzymes, the 3′ end fragments were trapped on Dyna beads (Dynal, Oslo, Norway) by means of their biotinylated tail, while the other fragments were washed away. After digestion with the second enzyme, the released restriction fragments were collected and used as templates in the subsequent AFLP steps. For pre-amplifications, a MseI primer without selective nucleotides was combined with a BstYI primer containing either a T or a C as 3′ most nucleotide. PCR conditions were as described (Vos et al., 1995). The obtained amplification mixtures were diluted 600-fold and 5 μl was used for selective amplifications using a P³³-labeled BstYI primer and the Amplitaq-Gold polymerase (Roche Diagnostics, Brussels, Belgium). Amplification products were separated on 5% polyacrylamide gels using the Sequigel system (Biorad). Dried gels were exposed to Kodak Biomax films as well as scanned in a PhosphorImager (Amersham Pharmacia Biotech, Little Chalfont, UK).

Bands corresponding to differentially expressed transcripts, among which the (partial) transcript corresponding to SEQ ID NO: 173, were isolated from the gel and eluted DNA was re-amplified under the same conditions as for selective amplification. Sequence information was obtained either by direct sequencing of the re-amplified polymerase chain reaction product with the selective BstYI primer or after cloning the fragments in pGEM-T easy (Promega, Madison, Wis.) and sequencing of individual clones. The obtained sequences were compared against nucleotide and protein sequences present in the publicly available databases by BLAST sequence alignments (Altschul et al., Nucleic Acids Res. 25 (17) 3389-3402 1997). When available, tag sequences were replaced with longer EST or isolated cDNA sequences to increase the chance of finding significant homology. The physical cDNA clone corresponding to SEQ ID NO 173 was subsequently amplified from a commercial tobacco cDNA library as follows:

A c-DNA library with an average size of inserts of 1,400 by was prepared from poly(A⁺) RNA isolated from actively dividing, non-synchronized BY2 tobacco cells. These library-inserts were cloned in the vector pCMVSPORT6.0, comprising an attB Gateway cassette (Life Technologies). From this library, 46,000 clones were selected, arrayed in 384-well microtiter plates, and subsequently spotted in duplicate on nylon filters. The arrayed clones were screened using pools of several hundreds of radioactively labelled tags as probes (including the BY2-tag corresponding to the sequence SEQ ID NO: 173). Positive clones were isolated (among which the clone corresponding to SEQ ID NO: 173), sequenced, and aligned with the tag sequence. Where the hybridisation with the tag failed, the full-length cDNA corresponding to the tag was selected by PCR amplification: tag-specific primers were designed using software commonly available and used in combination with a common vector primer to amplify partial cDNA inserts. Pools of DNA from 50,000, 100,000, 150,000, and 300,000 cDNA clones were used as templates in the PCR amplifications. Amplification products were then isolated from agarose gels, cloned, sequenced and their sequence aligned with those of the tags. Next, the full-length cDNA corresponding to the nucleotide sequence of SEQ ID NO 173 was cloned from the pCMVsport6.0 library vector into pDONR201, a Gateway® donor vector (Invitrogen, Paisley, UK) via a LR reaction, resulting in an entry clone.

The entry clone comprising SEQ ID NO: 171 or SEQ ID NO: 173 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice WSI18 promoter (SEQ ID NO: 175) for seed specific expression was located upstream of this Gateway cassette.

After the LR recombination step, the resulting expression vector pWSI18::GRP (FIG. 11) was transformed into Agrobacterium strain LBA4044 according to methods well known in the art.

In an alternative construct the rice GOS2 promoter (SEQ ID NO: 176) was used, resulting in the expression vector pGOS2::GRP.

### Example 26

### Plant Transformation

Transformation of plants was carried out according to the procedure outlined in Example 7.

### Example 27

### Phenotypic Evaluation Procedure

### 27.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are supplied with water at regular intervals to ensure that water and nutrients are not limiting to satisfy plant needs to complete growth and development.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds were in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### 27.2 Statistical Analysis: F Test

A two factor ANOVA (analysis of variants) was used as a statistical model for the overall evaluation of plant phenotypic characteristics. An F test was carried out an all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P values were obtained by comparing likelihood ratio test to chi square distributions.

### 27.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass.

The early vigour is the plant (seedling) aboveground area three weeks post-germination. Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 28

### Results of the Phenotypic Evaluation of the Transgenic Plants

The transgenic rice plants expressing the GRP nucleic acid represented by SEQ ID NO: 171 under control of the WSI18 promoter showed an increase of more than 5% for biomass, total weight of seeds, number of filled seeds, fill rate, and harvest index, when grown under conditions of drought stress. When grown under non-stress conditions, there was an increase of more than 5% observed for biomass, number of filled seeds, total weight of seeds, and number of first panicles.

For the construct with SEQ ID NO: 171 under control of the GOS2 promoter, an increase was observed in the transgenic plants for early vigour and for flowers per panicle, and for each these parameters, the increase was more than 5%.

Transgenic plants expressing SEQ ID NO: 173 under control of the WSI18 promoter and grown under conditions of drought stress, showed an increase in total weight of seeds, number of filled seeds, number of flowers per panicle, harvest index, and Thousand Kernel Weight. For Thousand Kernel Weight the observed increase was at least 3.5% and for the other parameters the increase was more than 5%.

### IV. Plant Homeodomain Finger-Homeodomain (PHDf-HD) Polypeptide

### Example 29

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid sequence or polypeptide sequences to sequence databases and by calculating the statistical significance of matches. For example, the polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid sequence (or polypeptide) sequences over a particular length. In some instances, the default parameters may be adjusted to modify the stringency of the search. For example the E-value may be increased to show less stringent matches. This way, short nearly exact matches may be identified.

Table D1 provides a list of nucleic acid sequences related to the PHDf-HDnucleic acid sequence used in the methods of the present invention.

TABLE D1
Examples of PHDf-HD polypeptides:

| | | Nucleic acid | Polypeptide | Database | |
|---|---|---|---|---|---|
| Name | Source organism | SEQ ID NO: | SEQ ID NO: | accession # | Status |
| Orysa_PHDf_HD | Oryza sativa | 179 | 180 | Os02g05450.1 | FL |
| | | | | NM_001052422 | |
| Arath_PHDf_HD_PRHA | Arabidopsis thaliana | 181 | 182 | At4g029940 | FL |
| | | | | NM_119140 | |
| Eucgr_PHDf_HD | Eucalyptus grandis | 183 | 184 | ADW17964 | FL |
| Medtr_PHDf_HD | Medicago truncatula | 185 | 186 | AC123547 | FL |
| Pinra_PHDf_HD | Pinus radiata | 187 | 188 | ADW18458 | FL |
| Poptr_PHDf_HD | Populus tremuloides | 189 | 190 | scaff_VI.625 | FL |
| Sacof_PHDf_HD | Saccharum officinarum | 191 | 192 | CA157855.1, | FL |
| | | | | CA261734.1, | |
| | | | | CA253314.1, | |
| | | | | CA220753.1, | |
| | | | | CA201958.1 | |
| Vitvi_PHDf_HD | Vitis vinifera | 193 | 184 | AM477372.2, | FL |
| | | | | AM488059.1 | |
| Zeama_PHDf_HD | Zea mays | 185 | 186 | EE162310, | FL |
| | | | | DN204182, | |
| | | | | CF057937 | |
| Arath_PHDf_HD_HAT3.1 | Arabidopsis thaliana | 187 | 188 | AT3G19510 | FL |
| | | | | NM_112838 | |
| Lotja_PHDf_HD | Lotus japonicus | 189 | 190 | AP006117.1 | FL |
| Orysa_PHDf_HD_HAZ1 | Oryza sativa | 191 | 192 | AB081340 | FL |
| | | | | Os06g12400.1 | |
| Petcr_PHDf_HD_PRHP | Petroselinum crispum | 193 | 194 | L21975 | FL |
| Zeama_PHDf_HD_HOX1a | Zea mays | 195 | 196 | X67561 | FL |
| Zeama_PHDf_HD_HOX1b | Zea mays | 197 | 198 | X92428 | FL |
| Zeama_PHDf_HD_HOX2a | Zea mays | 199 | 200 | X89760.1 | FL |
| Zeama_PHDf_HD_HOX2b | Zea mays | 201 | 202 | X89761 | FL |
| Vitvi_PHDf_HD_II | Vitis vinifera | 203 | 204 | AM464161.2 | FL |
| | | | | AM478203.2 | |
| Poptr_PHDf_HD_II | Populus tremuloides | 205 | 206 | scaff_IX.730 | FL |
| Poptr_PHDf_HD_III | Populus tremuloides | 207 | 208 | LG_I002624 | FL |
| Poptr_PHDf_HD_IV | Populus tremuloides | 209 | 210 | LG_XVIII1192 | FL |
| Ostta_PHDf_HD | Ostreococcus tauri | 211 | 212 | CR954214.4 | FL |
| Aqufor_PHDf_HD partial | Aquilegia formosa x | 213 | 214 | DR914726.1, | Partial |
| | Aquilegia pubescens | | | DR941696.1, | |
| | | | | DR943570.1 | |
| Glyma_PHDf_HD | Glycine max | 215 | 216 | Contig | Partial |
| | | | | GM06LC25006 | |
| Lotco_PHDf_HD | Lotus corniculatus | 217 | 218 | AP004517 | Partial |
| Sorpr_PHDf_HD 3′ | Sorghum propinquum | 219 | 220 | BF656332 | Partial |
| Sorpr_PHDf_HD 5′ | Sorghum propinquum | 221 | 222 | BF704605 | Partial |
| Phypa_PHDf_HD | Physcomitrella patens | 238 | 239 | XM_001762483 | FL |
| Phypa_PHDf_HD | Physcomitrella patens | 240 | 241 | XM_001779822 | FL |
| Zeama_PHDf_HD | Zea mays | 242 | 243 | | FL |

### Example 30

### Alignment of PHDf-HD Polypeptide Sequences

The University of Potsdam, Germany, has created plant transcription factor databases, including for Orysa sativa named riceTFOB. The polypeptide sequences corresponding to the transcription factors belonging to the HD family (120 gene models (91 loci) identified so far) were all downloaded, including the two PHDf-HD polypeptides (Os02g05450.1 and Os06g12400.1) identified to date. The polypeptide sequences of Table D1 of the present application were added (when full length, i.e. 21 polypeptide sequences) to the set of the HD family.

Alignment of all the polypeptide sequences was performed the Clustal algorithm (1.83) of progressive alignment, using default values (Thompson et al. (1997) Nucleic Acids Res 25:4876-4882; Chema et al. (2003). Nucleic Acids Res 31:3497-3500). A neighbour-joining tree was constructed thereafter, and is represented in FIG. 13 of the present application. The group of interest, comprising the two rice paralogs (Os02g05450.1 and Os06g12400.1) has been circled. Any polypeptide falling within this HD group (after a new multiple alignment step as described hereinabove) is considered to be useful in performing the methods of the invention as described herein.

In a multiple sequence alignment of the full length PHDf-HD polypeptides of Table D1, a number of features can be identified, and are marked in FIG. 17. From the N-terminus to the C-terminus of the polypeptides are: (i) a predicted nuclear localisation signal (NLS); (ii) a leucine zipper (ZIP), with four heptads (boxed in which usually a leucine (occasionally an isoleucine, a valine, or a methionine)) appears every seventh amino acid; (iii) a PHD finger (PHDf), with the typical C4HC3 (four cysteines, one histidine, three cysteines) with a characteristics cysteine spacing; (iv) an acidic stretch (rich in acidic amino acids D and E); (v) basic stretches (rich in basic amino acids K and R); (vi) a homeodomain (HD).

### Example 31

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table D2 for the global similarity and identity over the full length of the polypeptide sequences (excluding the partial polypeptide sequences). Percentage identity is given above the diagonal and percentage similarity is given below the diagonal.

The percentage identity between the PHDf-HD polypeptide sequences useful in performing the methods of the invention can be as low as 15% amino acid identity compared to SEQ ID NO: 180.

TABLE D2
MatGAT results for global similarity and identity over the full length of the
polypeptide sequences.

| | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|
| 1. Arath_PHDf_HD_PRHA | | 25 | 31 | 40 | 19 | 38 | 26 | 17 | 32 | 40 | 20 | 19 | 40 | 30 | 40 | 23 | 31 | 25 | 25 | 18 | 19 |
| 2. Arath_PHDf-HD_HAT3 | 44 | | 23 | 27 | 33 | 26 | 34 | 28 | 27 | 26 | 34 | 35 | 27 | 23 | 28 | 34 | 24 | 36 | 35 | 18 | 20 |
| 3. Orysa_PHDf_HD | 46 | 39 | | 35 | 18 | 34 | 20 | 18 | 31 | 34 | 17 | 18 | 33 | 56 | 36 | 21 | 57 | 21 | 23 | 15 | 15 |
| 4. Eucgr_PHDf_HD | 57 | 45 | 51 | | 21 | 47 | 27 | 20 | 36 | 50 | 22 | 21 | 51 | 32 | 52 | 25 | 32 | 26 | 27 | 17 | 17 |
| 5. Lotja_PHDf-HD | 36 | 47 | 31 | 35 | | 22 | 29 | 32 | 23 | 20 | 39 | 40 | 20 | 18 | 21 | 34 | 18 | 31 | 32 | 20 | 22 |
| 6. Medtr_PHDf_HD | 55 | 44 | 51 | 64 | 36 | | 26 | 19 | 35 | 50 | 20 | 20 | 49 | 30 | 50 | 25 | 32 | 27 | 26 | 17 | 17 |
| 7. Orysa_PHDf-HD_HAZ1 | 42 | 50 | 35 | 47 | 45 | 43 | | 26 | 25 | 25 | 29 | 29 | 26 | 21 | 26 | 32 | 21 | 50 | 46 | 29 | 30 |
| 8. Petcr_PHDf_HD_PRHP | 31 | 39 | 28 | 32 | 48 | 31 | 38 | | 22 | 20 | 32 | 33 | 20 | 18 | 20 | 30 | 18 | 26 | 25 | 19 | 21 |
| 9. Pinra_PHDf_HD | 53 | 44 | 47 | 51 | 42 | 50 | 44 | 39 | | 36 | 23 | 23 | 37 | 28 | 39 | 26 | 30 | 25 | 27 | 19 | 19 |
| 10. Poptr_PHDf_HD_I | 58 | 45 | 52 | 67 | 36 | 66 | 45 | 33 | 52 | | 20 | 21 | 84 | 30 | 55 | 25 | 31 | 25 | 25 | 17 | 17 |
| 11. Poptr_PHDf_HD_III | 36 | 47 | 30 | 34 | 56 | 35 | 44 | 46 | 41 | 36 | | 80 | 21 | 19 | 22 | 37 | 18 | 31 | 31 | 20 | 20 |
| 12. Poptr_PHDf_HD_II | 36 | 49 | 31 | 34 | 57 | 34 | 46 | 47 | 42 | 35 | 86 | | 21 | 18 | 22 | 38 | 18 | 30 | 30 | 20 | 21 |
| 13. Poptr_PHDf_HD_IV | 59 | 46 | 51 | 68 | 37 | 67 | 44 | 33 | 54 | 91 | 35 | 35 | | 30 | 57 | 25 | 32 | 25 | 25 | 17 | 17 |
| 14. Sacof_PHDf_HD | 48 | 43 | 69 | 51 | 31 | 50 | 38 | 29 | 45 | 51 | 34 | 33 | 51 | | 34 | 22 | 83 | 21 | 22 | 15 | 16 |
| 15. Vitvi_PHDf_HD_I | 58 | 47 | 52 | 69 | 36 | 67 | 43 | 32 | 52 | 74 | 36 | 35 | 75 | 52 | | 23 | 34 | 27 | 25 | 16 | 17 |
| 16. Vitvi_PHDf_HD_II | 39 | 47 | 36 | 39 | 56 | 39 | 45 | 47 | 43 | 40 | 56 | 57 | 40 | 36 | 41 | | 22 | 32 | 31 | 23 | 24 |
| 17. Zeama_PHDf_HD | 48 | 41 | 70 | 51 | 32 | 52 | 38 | 30 | 45 | 54 | 33 | 32 | 53 | 87 | 53 | 34 | | 22 | 22 | 15 | 15 |
| 18. Zeama_hox1a | 43 | 54 | 39 | 44 | 43 | 44 | 63 | 38 | 43 | 44 | 44 | 43 | 45 | 42 | 43 | 46 | 42 | | 83 | 24 | 24 |
| 19. Zeama_hox1b | 42 | 53 | 40 | 44 | 44 | 42 | 60 | 36 | 43 | 44 | 44 | 43 | 45 | 41 | 44 | 45 | 41 | 88 | | 23 | 23 |
| 20. Zeama_hox2a | 27 | 27 | 24 | 25 | 32 | 25 | 36 | 31 | 31 | 26 | 30 | 31 | 27 | 25 | 26 | 34 | 24 | 31 | 30 | | 81 |
| 21. Zeama_Hox2b | 29 | 28 | 23 | 27 | 33 | 27 | 37 | 34 | 31 | 27 | 32 | 33 | 26 | 25 | 27 | 35 | 25 | 32 | 30 | 86 | |

The percentage identity can be substantially increased if the identity calculation is performed between the conserved ZIP/PHDf domain (conserved leucine zipper/plant homeodomain finger domain, comprising the Zip and PHDf domains) of SEQ ID NO: 180 (as represented by SEQ ID NO: 233) and the conserved ZIP/PHDf domain of the polypeptides useful in performing the invention. The conserved ZIP/PHDf of SEQ ID NO: 233 is in total 180 contiguous amino acids long. Percentage identity over the conserved ZIP/PHDf domain amongst the polypeptide sequences useful in performing the methods of the invention ranges between 30% and 75% amino acid identity, as shown in Table D3.

TABLE D3
MatGAT results for global similarity and identity over the conserved ZIP/PHDf
domain amongst of the polypeptide sequences.

| | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|
| 1. ZIP/PHDf_Arath_PHDf_HD_PRHA | | 52 | 50 | 77 | 48 | 74 | 53 | 48 | 64 | 73 | 48 | 46 | 72 | 51 | 76 | 50 | 52 | 50 | 50 | 53 | 53 |
| 2. ZIP/PHDf_Arath_PHDf-HD_HAT3 | 70 | | 44 | 52 | 73 | 54 | 70 | 69 | 56 | 52 | 75 | 75 | 51 | 43 | 53 | 78 | 46 | 65 | 67 | 64 | 65 |
| 3. ZIP/PHDf_Orysa_PHDf_HD | 69 | 60 | | 56 | 39 | 54 | 37 | 39 | 56 | 53 | 39 | 38 | 53 | 70 | 59 | 41 | 71 | 37 | 38 | 34 | 33 |
| 4. ZIP/PHDf_Eucgr_PHDf_HD | 90 | 70 | 73 | | 52 | 79 | 48 | 50 | 71 | 82 | 51 | 49 | 81 | 55 | 84 | 52 | 56 | 48 | 48 | 49 | 48 |
| 5. ZIP/Lotja_PHDf-HD | 67 | 89 | 59 | 69 | | 52 | 64 | 69 | 52 | 47 | 76 | 76 | 47 | 37 | 50 | 78 | 38 | 64 | 66 | 61 | 61 |
| 6. ZIP/Medtr_PHDf_HD | 86 | 70 | 72 | 87 | 68 | | 52 | 49 | 66 | 74 | 49 | 48 | 75 | 50 | 79 | 51 | 53 | 51 | 51 | 52 | 52 |
| 7. ZIP/PHDf_Orysa_PHDf-HD_HAZ1 | 69 | 83 | 54 | 67 | 83 | 68 | | 64 | 51 | 47 | 68 | 67 | 47 | 35 | 47 | 68 | 37 | 79 | 80 | 82 | 84 |
| 8. ZIP/PHDf_Petcr_PHDf_HD_PRHP | 67 | 80 | 54 | 66 | 83 | 65 | 78 | | 53 | 47 | 71 | 73 | 45 | 39 | 48 | 74 | 42 | 62 | 63 | 62 | 62 |
| 9. ZIP/PHDf_Pinra_PHDf_HD | 82 | 74 | 72 | 83 | 73 | 82 | 70 | 69 | | 66 | 53 | 52 | 67 | 52 | 73 | 55 | 54 | 53 | 52 | 53 | 52 |
| 10. ZIP/PHDf_Poptr_PHDf_HD_I | 87 | 70 | 72 | 92 | 69 | 88 | 67 | 66 | 83 | | 48 | 47 | 93 | 53 | 82 | 50 | 55 | 49 | 48 | 49 | 49 |
| 11. ZIP/PHDf_Poptr_PHDf_HD_III | 66 | 87 | 57 | 67 | 90 | 65 | 82 | 82 | 70 | 67 | | 93 | 48 | 36 | 51 | 82 | 39 | 67 | 70 | 65 | 64 |
| 12. ZIP/PHDf_Poptr_PHDf_HD_II | 65 | 88 | 56 | 66 | 90 | 65 | 81 | 84 | 69 | 66 | 96 | | 46 | 35 | 49 | 83 | 38 | 67 | 69 | 64 | 64 |
| 13. ZIP/PHDf_Poptr_PHDf_HD_IV | 86 | 70 | 72 | 91 | 69 | 88 | 67 | 66 | 82 | 97 | 67 | 66 | | 52 | 82 | 48 | 54 | 47 | 47 | 48 | 47 |
| 14. ZIP/PHDf_Sacof_PHDf_HD | 71 | 60 | 82 | 73 | 57 | 69 | 55 | 57 | 70 | 72 | 56 | 55 | 72 | | 57 | 39 | 92 | 35 | 35 | 33 | 33 |
| 15. ZIP/PHDf_Vitvi_PHDf_HD_I | 89 | 71 | 73 | 90 | 70 | 88 | 67 | 64 | 84 | 91 | 67 | 66 | 89 | 73 | | 51 | 58 | 48 | 48 | 48 | 48 |
| 16. ZIP/PHDf_Vitvi_PHDf_HD_II | 67 | 88 | 58 | 69 | 91 | 68 | 84 | 84 | 73 | 69 | 90 | 93 | 67 | 57 | 69 | | 42 | 65 | 67 | 65 | 64 |
| 17. ZIP/PHDf_Zeama_PHDf_HD | 74 | 61 | 83 | 73 | 58 | 70 | 56 | 58 | 69 | 73 | 57 | 55 | 72 | 96 | 73 | 57 | | 38 | 37 | 35 | 34 |
| 18. ZIP/PHDf_Zeama_hox1a | 71 | 84 | 59 | 71 | 85 | 70 | 88 | 79 | 74 | 72 | 83 | 82 | 71 | 61 | 70 | 85 | 62 | | 94 | 74 | 76 |
| 19. ZIP/PHDf_Zeama_hox1b | 70 | 84 | 58 | 69 | 85 | 68 | 89 | 79 | 74 | 70 | 84 | 83 | 70 | 60 | 70 | 85 | 60 | 98 | | 75 | 78 |
| 20. ZIP/PHDf_Zeama_hox2a | 67 | 80 | 54 | 67 | 83 | 67 | 89 | 76 | 70 | 67 | 80 | 79 | 66 | 54 | 66 | 82 | 56 | 87 | 87 | | 92 |
| 21. ZIP/PHDf_Zeama_Hox2b | 67 | 82 | 53 | 66 | 83 | 67 | 90 | 77 | 71 | 66 | 80 | 79 | 67 | 53 | 67 | 82 | 55 | 88 | 88 | 97 | |

The percentage identity can also be calculated between the conserved HD of SEQ ID NO: 180 (as represented by SEQ ID NO: 234) and the conserved HD of the polypeptides useful in performing the invention. Percentage identity over the conserved HD amongst the polypeptide sequences useful in performing the methods of the invention ranges between 25% and 70% amino acid identity, as shown in Table D4.

TABLE D4
MatGAT results for global similarity and identity over the conserved HD amongst of
the polypeptide sequences.

| | | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|---|
| 1. | HD_Arath_PHDf_HD_PRHA | | 34 | 54 | 72 | 36 | 72 | 38 | 36 | 58 | 68 | 38 | 36 | 70 | 54 | 70 | 34 | 54 | 36 | 33 | 29 | 27 |
| 2. | HD_Arath_PHDf-HD_HAT3 | 56 | | 28 | 32 | 54 | 34 | 40 | 58 | 30 | 32 | 54 | 58 | 32 | 28 | 32 | 58 | 28 | 42 | 39 | 40 | 39 |
| 3. | HD_Orysa_PHDf_HD | 76 | 60 | | 68 | 42 | 58 | 42 | 36 | 46 | 64 | 38 | 40 | 66 | 94 | 62 | 42 | 92 | 38 | 35 | 35 | 33 |
| 4. | HD_Eucgr_PHDf_HD | 86 | 58 | 78 | | 42 | 72 | 44 | 42 | 60 | 82 | 38 | 38 | 84 | 66 | 76 | 42 | 66 | 44 | 41 | 35 | 33 |
| 5. | HD_Lotja_PHDf-HD | 60 | 72 | 66 | 64 | | 38 | 52 | 58 | 36 | 42 | 64 | 68 | 42 | 42 | 40 | 68 | 40 | 56 | 55 | 50 | 46 |
| 6. | HD_Medtr_PHDf_HD | 78 | 60 | 70 | 82 | 66 | | 36 | 36 | 62 | 68 | 38 | 36 | 70 | 58 | 66 | 36 | 58 | 40 | 37 | 27 | 27 |
| 7. | HD_Orysa_PHDf-HD_HAZ1 | 56 | 54 | 64 | 62 | 66 | 58 | | 44 | 26 | 44 | 50 | 48 | 44 | 42 | 40 | 46 | 42 | 70 | 65 | 73 | 71 |
| 8. | HD_Petcr_PHDf_HD_PRHP | 58 | 74 | 58 | 56 | 66 | 58 | 58 | | 36 | 42 | 54 | 58 | 42 | 36 | 38 | 62 | 36 | 50 | 45 | 40 | 39 |
| 9. | HD_Pinra_PHDf_HD | 74 | 54 | 64 | 72 | 58 | 72 | 50 | 52 | | 58 | 30 | 32 | 60 | 44 | 56 | 36 | 44 | 28 | 28 | 21 | 19 |
| 10. | HD_Poptr_PHDf_HD_I | 86 | 56 | 78 | 92 | 64 | 84 | 64 | 56 | 74 | | 36 | 36 | 98 | 64 | 72 | 42 | 64 | 42 | 39 | 35 | 33 |
| 11. | HD_Poptr_PHDf_HD_III | 54 | 72 | 62 | 60 | 82 | 58 | 66 | 66 | 54 | 58 | | 88 | 36 | 38 | 38 | 72 | 38 | 52 | 49 | 46 | 44 |
| 12. | HD_Poptr_PHDf_HD_II | 56 | 72 | 64 | 62 | 82 | 60 | 68 | 66 | 56 | 60 | 100 | | 36 | 40 | 38 | 78 | 40 | 50 | 47 | 46 | 44 |
| 13. | HD_Poptr_PHDf_HD_IV | 88 | 56 | 80 | 94 | 64 | 86 | 64 | 56 | 76 | 98 | 58 | 60 | | 66 | 72 | 42 | 66 | 42 | 39 | 35 | 33 |
| 14. | HD_Sacof_PHDf_HD | 74 | 60 | 94 | 76 | 64 | 72 | 62 | 56 | 62 | 76 | 60 | 62 | 78 | | 64 | 42 | 98 | 36 | 33 | 35 | 33 |
| 15. | HD_Vitvi_PHDf_HD_I | 82 | 60 | 80 | 86 | 70 | 80 | 60 | 58 | 74 | 88 | 62 | 64 | 88 | 82 | | 38 | 64 | 40 | 37 | 29 | 27 |
| 16. | HD_Vitvi_PHDf_HD_II | 64 | 76 | 66 | 66 | 82 | 68 | 64 | 74 | 56 | 66 | 84 | 84 | 66 | 64 | 68 | | 40 | 48 | 43 | 44 | 44 |
| 17. | HD_Zeama_PHDf_HD | 74 | 60 | 94 | 76 | 64 | 72 | 62 | 56 | 62 | 76 | 60 | 62 | 78 | 100 | 82 | 64 | | 36 | 33 | 35 | 33 |
| 18. | HD_Zeama_hox1a | 56 | 60 | 62 | 60 | 66 | 60 | 78 | 60 | 52 | 62 | 62 | 64 | 62 | 58 | 60 | 68 | 56 | | 86 | 60 | 56 |
| 19. | HD_Zeama_hox1b | 57 | 63 | 61 | 61 | 69 | 61 | 80 | 63 | 51 | 63 | 67 | 69 | 63 | 57 | 61 | 71 | 55 | 92 | | 59 | 53 |
| 20. | HD_Zeama_hox2a | 58 | 60 | 60 | 60 | 65 | 64 | 89 | 60 | 52 | 62 | 64 | 64 | 62 | 60 | 62 | 64 | 58 | 77 | 81 | | 90 |
| 21. | HD_Zeama_Hox2b | 56 | 56 | 56 | 56 | 62 | 58 | 85 | 54 | 48 | 58 | 60 | 60 | 58 | 56 | 58 | 62 | 54 | 71 | 77 | 92 | |

### Example 32

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Interpro is hosted at the European Bioinformatics Institute in the United Kingdom.

The results of the InterPro scan of the polypeptide sequence as represented by SEQ ID NO: 180 are presented in Table D5.

TABLE D5
InterPro scan results of the polypeptide sequence as represented by SEQ ID NO: 180

| | | | | Amino acid |
|---|---|---|---|---|
| | Integrated | Integrated | | coordinates on |
| InterPro | database | database | Integrated database | SEQ ID NO: |
| accession number | name | accession number | accession name | 180 |
| IPR0013556 | ProDom | PD000010 | PRH_ARATH_P48785 | 439-497 |
| Homeobox | | | | |
| IPR0013556 | PFAM | PF00046 | Homeobox | 439-495 |
| Homeobox | | | | |
| IPR0013556 | SMART | SM00389 | HOX | 438-500 |
| Homeobox | | | | |
| IPR0013556 | Profile | PS50071 | Homeobox_2 | 436-496 |
| Homeobox | | | | |
| IPR00195 | PFAM | PF00628 | PHD | 197-251 |
| Zinc-finger, PHD- | | | | |
| type | | | | |
| IPR00195 | SMART | SM00249 | PHD | 197-249 |
| Zinc-finger, PHD- | | | | |
| type | | | | |
| IPR00195 | Profile | PS50016 | ZF_PHD_2 | 195-251 |
| Zinc-finger, PHD- | | | | |
| type | | | | |
| IPR012287 | Gene3D | G3DSA:1.10.10.60 | No description | 436-501 |
| Homeodomain- | | | | |
| related | | | | |
| IPR013256 | SMART | SM00784 | No description | 78-154 |
| Chromatin SPT2 | | | | |
| Untegrated IPR | PANTHER | PTHR19418 | Homeobox protein | 397-414 |
| | | | | 431-590 |

The results of the InterPro scan clearly identifes the essential features of a PHDf-HD polypeptide, i.e., a PHDf zinc finger and homeobox, as represented for example respectively by Pfam entries PF00628 and PF00046.

HDs are known to have canonical residues. The HD of PHDf-HD polypeptides is highly divergent in sequence even at positions that are almost invariable among homeodomains. The sequence logo of the HD of the PHDf-HD polypeptides of Table D1, is shown in FIG. 15. Sequence logos are a graphical representation of an amino acid or nucleic acid multiple sequence alignment. Each logo consists of stacks of symbols, one stack for each position in the sequence. The overall height of the stack indicates the sequence conservation at that position, while the height of symbols within the stack indicates the relative frequency of each amino or nucleic acid at that position. In general, a sequence logo provides a richer and more precise description of, for example, a binding site, than would a consensus sequence. The algorithm (WebLogo) to produce such logos is available at the server of the University of California, Berkeley. The HD as represented by SEQ ID NO: 234, and comprised in SEQ ID NO: 180, is in accordance with the sequence logo as represented in FIG. 15. Polypeptides useful in performing the methods according to the invention comprise an HD comprised in the sequence logo as shown in FIG. 15.

### Example 33

### Prediction of Secondary Structure Features of the Polypeptide Sequences Useful in Performing the Methods of the Invention

Coiled coils usually contain a repeated seven amino acid residue pattern called heptad repeats. Coiled coils are important to identify for protein-protein interactions, such as oligomerization, either of identical proteins, of proteins of the same family, or of unrelated proteins. Recently much progress has been made in computational prediction of coiled coils from sequence data. Many algorithms well known to a person skilled in the art are available at the ExPASy Proteomics tools. One of them, COILS, is a program that compares a sequence to a database of known parallel two-stranded coiled-coils and derives a similarity score. By comparing this score to the distribution of scores in globular and coiled-coil proteins, the program then calculates the probability that the sequence will adopt a coiled-coil conformation.

The PHDf-HD polypeptide as represented by SEQ ID NO: 180, has two N-terminal predicted coiled coil domains, with a high probability, in all three windows (14, 21 and 28) examined. In Table D6, the residue coordinates, residues, the three windows and corresponding probability values are shown. In FIG. 16, is the graphical output of the COILS algorithm on the polypeptide as represented by SEQ ID NO: 180, where the two predicted coiled coils are clearly visible in the N-terminal half of the polypeptide, in all three windows (as represented by the three lines).

TABLE D6
Numerical output of the COILS algorithm on the polypeptide as represented
by SEQ ID NO: 180. The residue coordinates (#), residues, the three
windows and corresponding probability values are shown. Probabilities
above 0.09 are shown in grey.

| # | Residue | Window = 14 | Prob | Window = 21 | Prob | Window = 28 | Prob |
|---|---|---|---|---|---|---|---|
| 87 | P | d | 0.001 | e | 0.001 | E | 0.001 |
| 88 | T | e | 0.063 | f | 0.274 | F | 0.099 |
| 89 | R | f | 0.063 | g | 0.567 | G | 0.099 |
| 90 | R | g | 0.063 | a | 0.567 | A | 0.099 |
| 91 | K | b | 0.066 | b | 0.642 | B | 0.188 |
| 92 | H | c | 0.066 | c | 0.642 | C | 0.188 |
| 93 | K | d | 0.069 | d | 0.642 | D | 0.591 |
| 94 | Q | e | 0.178 | e | 0.642 | E | 0.672 |
| 95 | K | f | 0.335 | f | 0.642 | F | 0.672 |
| 96 | R | g | 0.335 | g | 0.642 | G | 0.772 |
| 97 | K | a | 0.335 | a | 0.642 | A | 0.772 |
| 98 | N | b | 0.347 | b | 0.642 | B | 0.772 |
| 99 | D | c | 0.347 | c | 0.642 | C | 0.772 |
| 100 | E | d | 0.347 | d | 0.642 | D | 0.772 |
| 101 | S | e | 0.347 | e | 0.642 | E | 0.808 |
| 102 | D | f | 0.347 | f | 0.642 | F | 0.808 |
| 103 | E | g | 0.347 | g | 0.642 | g | 0.808 |
| 104 | V | a | 0.347 | a | 0.642 | a | 0.808 |
| 105 | S | b | 0.347 | b | 0.642 | b | 0.808 |
| 106 | R | c | 0.347 | c | 0.642 | c | 0.808 |
| 107 | M | d | 0.347 | d | 0.642 | d | 0.808 |
| 108 | E | e | 0.347 | e | 0.642 | e | 0.808 |
| 109 | K | f | 0.347 | f | 0.642 | F | 0.808 |
| 110 | R | g | 0.347 | g | 0.642 | g | 0.808 |
| 111 | A | a | 0.347 | a | 0.642 | a | 0.808 |
| 112 | R | b | 0.307 | b | 0.514 | b | 0.808 |
| 113 | Y | c | 0.066 | c | 0.503 | c | 0.808 |
| 114 | L | d | 0.262 | d | 0.503 | d | 0.808 |
| 115 | L | e | 0.262 | e | 0.503 | e | 0.808 |
| 116 | I | f | 0.262 | f | 0.503 | F | 0.808 |
| 117 | K | g | 0.262 | g | 0.503 | g | 0.808 |
| 118 | I | a | 0.262 | a | 0.503 | a | 0.808 |
| 119 | K | b | 0.262 | b | 0.503 | b | 0.808 |
| 120 | Q | c | 0.262 | c | 0.503 | c | 0.808 |
| 121 | E | d | 0.262 | d | 0.503 | d | 0.808 |
| 122 | Q | e | 0.262 | e | 0.503 | e | 0.808 |
| 123 | N | f | 0.262 | f | 0.503 | F | 0.808 |
| 124 | L | g | 0.262 | g | 0.434 | g | 0.808 |
| 125 | L | a | 0.262 | a | 0.434 | a | 0.808 |
| 126 | D | b | 0.262 | b | 0.434 | b | 0.808 |
| 127 | A | c | 0.262 | c | 0.304 | c | 0.808 |
| 128 | Y | d | 0.145 | d | 0.304 | d | 0.808 |
| 129 | S | e | 0.145 | e | 0.044 | e | 0.808 |
| 130 | G | f | 0.119 | f | 0.009 | F | 0.367 |
| 131 | D | g | 0.078 | g | 0.007 | g | 0.136 |
| 132 | G | a | 0.002 | a | 0.001 | a | 0.005 |
| 133 | W | b | 0 | a | 0.001 | a | 0.001 |
| 134 | N | b | 0.001 | b | 0.045 | b | 0.057 |
| 135 | G | c | 0.001 | c | 0.045 | c | 0.057 |
| 136 | H | d | 0.003 | d | 0.07 | d | 0.219 |
| 137 | S | e | 0.008 | e | 0.132 | e | 0.231 |
| 138 | R | f | 0.025 | f | 0.357 | F | 0.231 |
| 139 | E | g | 0.025 | g | 0.357 | g | 0.231 |
| 140 | K | a | 0.025 | a | 0.357 | a | 0.231 |
| 141 | I | b | 0.028 | b | 0.357 | b | 0.231 |
| 142 | K | c | 0.07 | c | 0.357 | c | 0.462 |
| 143 | P | d | 0.07 | d | 0.357 | d | 0.462 |
| 144 | E | e | 0.998 | e | 0.997 | e | 0.974 |
| 145 | K | f | 0.998 | f | 0.997 | f | 0.974 |
| 146 | E | g | 0.998 | g | 0.997 | g | 0.974 |
| 147 | L | a | 0.998 | a | 0.997 | a | 0.974 |
| 148 | Q | b | 0.998 | b | 0.997 | b | 0.974 |
| 149 | R | c | 0.998 | c | 0.997 | c | 0.974 |
| 150 | A | d | 0.998 | d | 0.997 | d | 0.974 |
| 151 | K | e | 0.998 | e | 0.997 | e | 0.974 |
| 152 | K | f | 0.998 | f | 0.997 | f | 0.974 |
| 153 | Q | g | 0.998 | g | 0.997 | g | 0.974 |
| 154 | I | a | 0.998 | a | 0.997 | a | 0.974 |
| 155 | M | b | 0.998 | b | 0.997 | b | 0.974 |
| 156 | K | c | 0.998 | c | 0.997 | c | 0.974 |
| 157 | Y | d | 0.998 | d | 0.997 | d | 0.974 |
| 158 | K | e | 0.989 | e | 0.997 | e | 0.974 |
| 159 | I | f | 0.896 | f | 0.997 | f | 0.974 |
| 160 | A | g | 0.486 | g | 0.997 | g | 0.974 |
| 161 | I | a | 0.409 | a | 0.997 | a | 0.974 |
| 162 | R | b | 0.274 | b | 0.997 | b | 0.974 |
| 163 | D | c | 0.255 | c | 0.997 | c | 0.974 |
| 164 | V | d | 0.095 | d | 0.997 | d | 0.974 |
| 165 | I | e | 0.025 | e | 0.871 | e | 0.974 |
| 166 | H | f | 0.025 | f | 0.622 | f | 0.974 |
| 167 | Q | g | 0.025 | g | 0.496 | g | 0.974 |
| 168 | L | a | 0.025 | a | 0.496 | a | 0.974 |
| 169 | D | b | 0.025 | b | 0.468 | b | 0.974 |
| 170 | L | c | 0.005 | c | 0.111 | c | 0.974 |
| 171 | C | d | 0.002 | d | 0.02 | d | 0.974 |
| 172 | S | e | 0.001 | e | 0.007 | e | 0.719 |
| 173 | S | f | 0.001 | f | 0.003 | f | 0.482 |
| 174 | S | g | 0.001 | g | 0.001 | g | 0.066 |
| 175 | G | a | 0 | a | 0 | a | 0.001 |

Another coiled coil is predicted in the C-terminal half of the PHDf-HD polypeptide as represented by SEQ ID NO: 180, with a lower probability than the two coiled domains comprised in the N-terminal half of the polypeptide.

### Example 34

### Subcellular Localisation Prediction of the Polypeptide Sequences Useful in Performing the Methods of the Invention

LOCtree is an algorithm that can predict the subcellular localization and DNA-binding propensity of non-membrane proteins in non-plant and plant eukaryotes as well as prokaryotes. LOCtree classifies eukaryotic animal proteins into one of five subcellular classes, while plant proteins are classified into one of six classes and prokaryotic proteins are classified into one of three classes.

Whenever available, LOCtree also reports predictions based on the following: 1) Nuclear localization signals found by the PredictNLS algorithm, 2) Localization inferred using Prosite motifs and Pfam domains found in the protein, and 3) SWISS-PROT keywords associated with a protein. Localization is inferred in the last two cases using the entropy-based LOCkey algorithm. The software is hosted at the University of Columbia, USA.

Motif and keyword based prediction of subcellular localization of a PHDf-HD polypeptide as represented by SEQ ID NO: 180, using LOCkey:

| Predicted | | Alternative | SWISS-PROT keywords used |
|---|---|---|---|
| Localization | Confidence | prediction | to assign localization |
| Nuclear | 100 | — | Homeobox, DNA-binding, |
| | | | Transcription regulation, |
| | | | Nuclear protein, |
| | | | Transcription, Zinc-finger |

Prediction of a nuclear localisation signal (NLS) is done using PredictNLS algorithm, for example. The algorithm is also hosted by the server at the University of Columbia, USA. In the Table below, prediction of NLS on the polypeptide of SEQ ID NO: 180 using the PredictNLS algorithm, is shown.

| | | Coordinates on |
|---|---|---|
| Generalized motif ([KR]{3,5} | Hit on polypeptide of SEQ ID NO: | polypeptide of |
| between 3 and 5 times K or R) | 180 | SEQ ID NO: 180 |
| K[RK]{3,5}x{11,18}[RK]Kx{2,3}K | KRRRGSDAATGKSATGPTRRKHKQK | 71-95 |
| [KR]{4}x{20,24}K{1,4}xK | KRRRGSDAATGKSATGPTRRKHKQKRK | 71-97 |

In the polypeptide sequence below (SEQ ID NO: 180), the position of the predicted NLS is shown in bold and underlined twice:

MNTPEKKPLCYTSRRALQQRTESSSELISVSKRATRQNTPRKPDSPPK
RTTRSSANLAKCIENKHHSSPLKRRRGSDAATGKSATGPTRRKHK
QKRKNDESDEVSRMEKRARYLLIKIKQEQNLLDAYSGDGWNGHSRE
KIKPEKELQRAKKQIMKYKIAIRDVIHQLDLCSSSGSKDDSVIPPDGCHE
SVNPEHTICSRCKSHESFPDNNIIFCEGGCKLACHQKCLEPPFDKILPT
TRHGRLCKHCSSKMKILDAINAHLGTSFTVKCPSSDIFKEAAEHFNSDD
GLGQDWLSEYSGDEDYDPEENEASSSGEENKSADSNCSGSPLYSPNDD
IPDFISADFNDAEGFCRESSNLGIDFGEDGLAEILTHQRPRRDVDYTQLN
EQMFGEPIGNDEQSEDEDWGLNKRKKRRTGSTGVGTNSVEGRSDVKS
NKKAQPRRKLFRIPPAAVEVLRKAFAENELPARSVKENLSTELGISFEK
IDKWFKNTRCAALRDRKGESRYSGPSKRSRTSIEKAETSAKVDQMDNSCF
LPLSEIINVPTRLQKGLDKKPKSINSPPRPQDNETCLSPTDKTKEGTPPT
IKPSITDSSQLMNNDIGTEETAVSWVDTWASDALHFLDVSDDEHFFDV
IEKVCGLENRLQRLKENMLSSSSSTDNNVAAESGLQNEVVLVPAAEL
KDKAS

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 35

### Cloning of Nucleic Acid Sequence as Represented by SEQ ID NO: 179

Unless otherwise stated, recombinant DNA techniques were performed according to standard protocols described in (Sambrook (2001) Molecular Cloning: a laboratory manual, 3rd Edition Cold Spring Harbor Laboratory Press, CSI-1, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Oryza sativa PHDf-HD gene was amplified by PCR using as template a rice cDNA bank synthesized from mRNA extracted from mixed plant tissues. Primer prm09687 (SEQ ID NO: 236; sense: 5′-ggggacaagtttgtacaaaaaagcaggcttaaacaatgaataccccagaaaa gaaa-3′) and primer prm09688 SEQ ID NO: 237; reverse, complementary: 5′-ggggaccacfttgtacaagaaagctgggtgatgcaaggttaagatgcttt-3′), which include the AttB sites for Gateway recombination, were used for PCR amplification. PCR was performed using Hifi Tag DNA polymerase in standard conditions. A PCR fragment of the expected length (including attB sites) was amplified and purified also using standard methods. The first step of the Gateway procedure, the BP reaction, was then performed, during which the PCR fragment recombined in vivo with the pDONR201 plasmid to produce, according to the Gateway terminology, an “entry clone”. Plasmid pDONR201 was purchased from Invitrogen, as part of the Gateway® technology.

### Example 36

### Expression Vector Construction Using the Nucleic Acid Sequence as Represented by SEQ ID NO: 179

The entry clone comprising SEQ ID NO: 179 was subsequently used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 235) for constitutive expression was located upstream of this Gateway cassette.

After the LR recombination step, the resulting expression vector pGOS2::PHDf-HD and (FIG. 18) was transformed into Agrobacterium strain LBA4044 according to methods well known in the art.

### Example 37

### Plant Transformation

### Rice Transformation

The Agrobacterium containing the expression vectors were used independently to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were subcultured on fresh medium 3 days before co-cultivation (to boost cell division activity).

Agrobacterium strain LBA4404 containing each individual expression vector was used independently for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for each construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al., 1994).

### Example 38

### Phenotypic Evaluation Procedure

### 38.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are supplied with water at regular intervals to ensure that water and nutrients are not limiting to satisfy plant needs to complete growth and development.

From the stage of sowing until the stage of maturity the plants were passed several times through a digital imaging cabinet. At each time point digital images (2048×1536 pixels, 16 million colours) were taken of each plant from at least 6 different angles.

### 38.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

38.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles. The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed weight per plant was measured by weighing all filled husks harvested from one plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed weight per plant and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

Reduced nutrient (nitrogen) availability screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Example 39

### Results of the Phenotypic Evaluation of the Transgenic Rice Plants

The results of the evaluation of transgenic rice plants expressing the nucleic acid sequence encoding a PHDf-HD polypeptide as represented by SEQ ID NO: 180, under the control of the GOS2 promoter for constitutive expression, are presented below.

There was a significant increase in the number of panicles, in the total seed yield per plant, in the total number of filled seeds, in the total number of seeds, in the Thousand Kernel Weight (TKW), and in the harvest index of the transgenic plants compared to corresponding nullizygotes (controls), as shown in Table D7.

TABLE D7
Results of the evaluation of transgenic rice plants expressing the
nucleic acid sequence encoding a PHDf-HD polypeptide as
represented by SEQ ID NO: 180, under the control of the GOS2
promoter for constitutive expression.

| | Average % increase in 3 |
|---|---|
| | events in the T1 generation |
| Number of first panicles | 22% |
| Total seed yield per plant | 30% |
| Total number of filled seeds | 24% |
| Total number of seeds | 24% |
| TKW | 4% |
| Harvest index | 17% |

### Example 40

### Results of the Phenotypic Evaluation of the Transgenic Rice Plants Grown Under Reduced Nutrient Availability

The results of the evaluation of transgenic rice plants expressing the nucleic acid sequence encoding a PHDf-HD polypeptide as represented by SEQ ID NO: 180, under the control of the GOS2 promoter for constitutive expression, and grown under reduced nutrient availability, are presented below.

There was a significant increase in biomass, in emergeance vigor, in the total seed yield per plant, in the total number of filled seeds, in the seed filling rate, in the total number of seeds, in the Thousand Kernel Weight (TKW), and in the harvest index of the transgenic plants compared to corresponding nullizygotes (controls), as shown in Table D8.

TABLE D8
Results of the evaluation of transgenic rice plants expressing the
nucleic acid sequence encoding a PHDf-HD polypeptide as represented
by SEQ ID NO: 180, under the control of the GOS2 promoter for
constitutive expression, and grown under reduced nutrient availability.

| | Overall average % increase |
|---|---|
| | (6 events in the T2 generation) |
| Biomass | 8% |
| Emergeance vigor | 20% |
| Total seed yield per plant | 17% |
| Total number of filled seeds | 12% |
| Seed filling rate | 3% |
| Total number of seeds | 9% |
| TKW | 5% |
| Harvest index | 9% |

### Example 41

### Examples of Transformation of Other Crops

Transformation of corn, wheat, soybean, rapeseed/canola, alfalfa and cotton was carried out as outlined in Example 7

### Example 42

### Examples of Abiotic Stress Screens

### Drought Screen

Plants from a selected number of events are grown in potting soil under normal conditions until they approached the heading stage. They are then transferred to a “dry” section where irrigation is withheld. Humidity probes are inserted in randomly chosen pots to monitor the soil water content (SWC). When SWC go below certain thresholds, the plants are automatically re-watered continuously until a normal level is reached again. The plants are then re-transferred to normal conditions. The rest of the cultivation (plant maturation, seed harvest) is the same as for plants not grown under abiotic stress conditions. Growth and yield parameters are recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants were harvested. Growth and yield parameters are recorded as detailed for growth under normal conditions.

VIII. bHLH11-Like (Basic Helix-Loop-Helix 11) Protein

### Example 43

### Identification of Sequences Related to the bHLH11-Like Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified. Table E1 provides a list of nucleic acid sequences related to the nucleic acid sequence used in the methods of the present invention.

TABLE E1
Examples of bHLH11-like polypeptides:

| | Nucleic acid | Protein |
|---|---|---|
| Plant Source | SEQ ID NO: | SEQ ID NO: |
| Triticum aestivum | 244 | 245 |
| Allium cepa | 258 | 327 |
| Arabidopsis thaliana | 259 | 328 |
| Arabidopsis thaliana | 260 | 329 |
| Arabidopsis thaliana | 261 | 330 |
| Arabidopsis thaliana | 262 | 331 |
| Arabidopsis thaliana | 263 | 332 |
| Aquilegia vulgaris | 264 | 333 |
| Aquilegia vulgaris | 265 | 334 |
| Brassica napus | 266 | 335 |
| Citrus clementina | 267 | 336 |
| Curcuma longa | 268 | 337 |
| Citrus paridisi hybrid | 269 | 338 |
| Citrus sinensis | 270 | 339 |
| Eucalyptus grandis | 271 | 340 |
| Eucalyptus grandis | 272 | 341 |
| Gossypium hirsutum | 273 | 342 |
| Gossypium hirsutum | 274 | 343 |
| Gossypium hirsutum | 275 | 344 |
| Gossypium hirsutum | 276 | 345 |
| Gossypium hirsutum | 277 | 346 |
| Gossypium hirsutum | 278 | 347 |
| Glycine max | 279 | 348 |
| Glycine max | 280 | 349 |
| Glycine max | 281 | 350 |
| Glycine max | 282 | 351 |
| Gossypium raimondii | 283 | 352 |
| Helianthus petiolaris | 284 | 353 |
| Hordeum vulgare | 285 | 354 |
| Lactuca perennis | 286 | 355 |
| Nicotiana benthamiana | 287 | 356 |
| Nicotiana benthamiana | 288 | 357 |
| Nicotiana benthamiana | 289 | 358 |
| Nicotiana benthamiana | 290 | 359 |
| Nicotiana tabacum | 291 | 360 |
| Oryza sativa | 292 | 361 |
| Oryza sativa | 293 | 362 |
| Oryza sativa | 294 | 363 |
| Oryza sativa | 295 | 364 |
| Oryza sativa | 296 | 365 |
| Oryza sativa | 297 | 366 |
| Oryza sativa | 298 | 367 |
| Picea abies | 299 | 368 |
| Populus deltoides | 300 | 369 |
| Pinus radiata | 301 | 370 |
| Picea sitchensis | 302 | 371 |
| Pinus taeda | 303 | 372 |
| Populus trichocarpa | 304 | 373 |
| Populus trichocarpa | 305 | 374 |
| Populus trichocarpa | 306 | 375 |
| Populus trichocarpa | 307 | 376 |
| Populus trichocarpa | 308 | 377 |
| Poncirus trifoliata | 309 | 378 |
| Ricinus communis | 310 | 379 |
| Ricinus communis | 311 | 380 |
| Sorghum bicolor | 312 | 381 |
| Solanum lycopersicum | 313 | 382 |
| Solanum lycopersicum | 314 | 383 |
| Solanum lycopersicum | 315 | 384 |
| Solanum tuberosum | 316 | 385 |
| Solanum tuberosum | 317 | 386 |
| Solanum tuberosum | 318 | 387 |
| Solanum tuberosum | 319 | 388 |
| Triticum aestivum | 320 | 389 |
| Vitis vinifera | 321 | 390 |
| Vitis vinifera | 322 | 391 |
| Vitis vinifera | 323 | 392 |
| Vitis vinifera | 324 | 393 |
| Zea mays | 325 | 394 |
| Zea mays | 326 | 395 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### Example 44

### Alignment of bHLH11-Like Polypeptide Sequences

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0,1 and the selected weight matrix is Blosum 62 (if polypeptides are aligned). Minor manual editing was done to further optimise the alignment. Sequence conservation among bHLH11-like polypeptides is essentially in the C-terminal bHLH domain of the polypeptides, the N-terminal domain usually being more variable in sequence length and composition. The bHLH11-like polypeptides are aligned in FIG. 21.

A phylogenetic tree of bHLH11-like polypeptides (FIG. 22) was constructed using “CLUSTALX”, and a neighbour-joining tree was calculated. The circular cladogram was drawn using Dendroscope (Huson et al., 2007).

### Example 45

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table E2 for the global similarity and identity over the full length of the polypeptide sequences. Percentage identity is given above the diagonal and percentage similarity is given below the diagonal. SEQ ID NO: 245 is represented as TabHLH11.

The percentage identity between the bHLH11-like polypeptide sequences useful in performing the methods of the invention can be as low as 20% amino acid identity compared to SEQ ID NO: 245. The identity is however much higher when the HLH domains are compared (Table E3).

TABLE E2
MatGAT results for global similarity and identity over the full length of the bHLH11-like polypeptide sequences.

| | | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| 1. | TC3698 | | 51.1 | 53.5 | 15.9 | 39.5 | 37.1 | 49.4 | 19.5 | 53.8 | 45.8 | 36.3 | 53.7 | 30.6 | 52.0 | 46.5 | 51.0 |
| 2. | AT1G03040 | 63.2 | | 81.0 | 16.3 | 37.8 | 36.3 | 52.4 | 19.9 | 72.3 | 40.5 | 35.9 | 64.6 | 31.1 | 50.7 | 59.5 | 45.9 |
| 3. | AT4G02590 | 64.2 | 89.0 | | 17.0 | 37.0 | 35.1 | 55.2 | 18.8 | 82.4 | 43.0 | 35.4 | 69.5 | 33.8 | 53.5 | 64.6 | 47.6 |
| 4. | AT2G24260 | 21.7 | 22.0 | 22.3 | | 27.7 | 19.7 | 16.4 | 12.7 | 15.6 | 17.0 | 19.0 | 15.8 | 23.0 | 15.8 | 15.8 | 16.8 |
| 5. | AT4G30980 | 53.2 | 53.9 | 52.6 | 30.6 | | 46.4 | 36.0 | 22.0 | 35.5 | 38.7 | 40.6 | 35.1 | 41.3 | 36.3 | 32.6 | 36.2 |
| 6. | AT5G58010 | 53.9 | 55.0 | 52.6 | 23.8 | 58.1 | | 32.6 | 24.2 | 34.0 | 35.4 | 37.1 | 36.0 | 35.7 | 31.6 | 32.3 | 35.5 |
| 7. | TC15501 | 58.4 | 64.1 | 65.5 | 22.1 | 49.9 | 48.1 | | 17.5 | 52.8 | 47.0 | 37.0 | 56.0 | 35.1 | 57.8 | 59.2 | 52.8 |
| 8. | TC19278 | 31.9 | 31.1 | 30.0 | 15.6 | 33.2 | 35.0 | 26.8 | | 18.8 | 21.1 | 16.4 | 19.5 | 18.9 | 17.8 | 17.6 | 17.5 |
| 9. | TC10015_part | 64.9 | 80.5 | 85.8 | 19.9 | 50.6 | 52.2 | 60.4 | 31.7 | | 41.9 | 35.6 | 72.5 | 30.8 | 52.5 | 59.7 | 47.1 |
| 10. | DY268946 | 56.8 | 55.9 | 59.1 | 23.8 | 52.5 | 46.4 | 61.0 | 31.6 | 53.0 | | 34.5 | 42.7 | 32.9 | 52.7 | 43.2 | 50.8 |
| 11. | TA2544 | 47.3 | 46.8 | 47.8 | 26.1 | 49.3 | 46.3 | 49.0 | 23.9 | 45.6 | 47.6 | | 35.0 | 44.9 | 38.4 | 35.9 | 36.4 |
| 12. | TA3392 | 68.1 | 76.8 | 79.4 | 21.7 | 51.9 | 52.2 | 63.0 | 32.7 | 84.0 | 56.2 | 43.7 | | 30.9 | 53.0 | 68.7 | 49.4 |
| 13. | TA12416 | 38.0 | 38.2 | 40.7 | 30.4 | 48.4 | 43.3 | 46.3 | 25.8 | 37.3 | 43.7 | 54.6 | 40.3 | | 32.2 | 32.4 | 33.1 |
| 14. | WO051050seqID77 | 61.3 | 63.8 | 66.3 | 20.8 | 51.3 | 50.9 | 66.4 | 28.1 | 61.9 | 65.8 | 45.4 | 61.3 | 39.4 | | 53.2 | 58.7 |
| 15. | WO051050seqID1671 | 56.9 | 70.5 | 74.9 | 22.3 | 46.5 | 47.1 | 70.9 | 27.5 | 68.5 | 59.0 | 47.3 | 75.4 | 42.0 | 64.5 | | 46.8 |
| 16. | TA55042 | 62.5 | 59.5 | 62.8 | 22.4 | 51.7 | 52.0 | 65.8 | 27.8 | 58.0 | 67.0 | 48.8 | 58.9 | 42.4 | 69.2 | 62.7 | |
| 17. | TC207545 | 60.7 | 66.6 | 68.1 | 20.1 | 51.0 | 51.5 | 57.0 | 38.5 | 71.6 | 53.9 | 42.9 | 74.7 | 36.7 | 56.9 | 64.5 | 56.8 |
| 18. | TA13791 | 63.7 | 80.5 | 82.6 | 22.1 | 52.6 | 54.8 | 68.1 | 31.7 | 78.2 | 59.4 | 48.3 | 83.5 | 43.1 | 65.0 | 78.0 | 63.1 |
| 19. | CO123623 | 61.4 | 57.3 | 58.7 | 19.3 | 49.7 | 53.9 | 58.1 | 32.8 | 62.6 | 64.3 | 42.9 | 65.1 | 38.2 | 65.6 | 55.2 | 65.6 |
| 20. | DT543504 | 52.0 | 55.2 | 54.8 | 22.3 | 51.3 | 52.6 | 47.3 | 37.9 | 49.7 | 50.4 | 42.7 | 55.2 | 39.7 | 51.9 | 51.4 | 50.5 |
| 21. | TC60118 | 63.4 | 80.2 | 82.3 | 22.0 | 52.3 | 54.5 | 67.8 | 31.7 | 77.9 | 59.1 | 48.0 | 83.2 | 42.9 | 64.7 | 77.7 | 62.8 |
| 22. | TC60119 | 65.1 | 78.9 | 82.9 | 21.1 | 51.9 | 53.3 | 67.0 | 30.6 | 77.3 | 58.6 | 47.6 | 82.6 | 40.5 | 65.6 | 76.6 | 60.7 |
| 23. | TC61833 | 63.6 | 78.4 | 81.9 | 21.9 | 54.2 | 55.4 | 66.1 | 30.2 | 77.0 | 56.8 | 47.6 | 82.3 | 40.7 | 65.0 | 76.9 | 62.5 |
| 24. | TC67603 | 40.3 | 43.3 | 42.4 | 28.8 | 48.7 | 45.9 | 47.5 | 26.9 | 40.7 | 45.7 | 56.7 | 42.9 | 67.0 | 43.1 | 42.9 | 45.7 |
| 25. | TC229602 | 56.8 | 60.3 | 61.0 | 18.9 | 45.8 | 49.2 | 50.7 | 45.2 | 63.3 | 49.9 | 38.0 | 65.1 | 33.5 | 52.5 | 57.5 | 52.3 |
| 26. | TC205173 | 50.4 | 51.0 | 48.7 | 25.3 | 58.5 | 54.6 | 53.3 | 34.3 | 47.2 | 52.5 | 52.2 | 47.5 | 54.6 | 51.3 | 48.0 | 54.6 |
| 27. | TA1140 | 63.4 | 64.1 | 64.5 | 21.5 | 55.2 | 53.9 | 64.7 | 31.0 | 62.7 | 55.1 | 45.1 | 63.1 | 42.2 | 65.3 | 60.1 | 64.0 |
| 28. | TC140470 | 37.6 | 38.5 | 38.0 | 26.6 | 35.8 | 36.4 | 41.2 | 22.2 | 34.9 | 37.0 | 43.7 | 37.4 | 48.0 | 38.0 | 40.3 | 40.3 |
| 29. | TA3490_part | 67.6 | 64.6 | 64.2 | 22.0 | 53.2 | 54.5 | 62.1 | 32.4 | 65.2 | 58.0 | 45.6 | 67.9 | 41.4 | 63.4 | 59.5 | 63.4 |
| 30. | CK293938 | 67.6 | 64.2 | 65.2 | 20.6 | 53.9 | 54.2 | 61.5 | 32.1 | 65.2 | 60.0 | 45.6 | 66.2 | 41.4 | 68.4 | 60.1 | 64.0 |
| 31. | TC8633 | 64.6 | 78.8 | 79.0 | 22.5 | 53.9 | 56.3 | 68.1 | 31.8 | 74.5 | 56.5 | 45.1 | 75.5 | 41.8 | 65.3 | 71.1 | 62.5 |
| 32. | TC7102 | 39.7 | 42.4 | 42.9 | 30.0 | 49.2 | 44.2 | 46.3 | 25.1 | 39.7 | 44.7 | 56.9 | 40.6 | 69.7 | 43.6 | 44.2 | 43.6 |
| 33. | TC7103 | 45.0 | 46.0 | 46.3 | 26.7 | 53.2 | 51.2 | 48.8 | 27.9 | 45.0 | 46.5 | 57.1 | 45.2 | 65.0 | 46.3 | 50.4 | 49.4 |
| 34. | TC12771 | 66.1 | 64.9 | 65.5 | 21.2 | 54.8 | 54.4 | 61.5 | 30.9 | 64.4 | 59.7 | 45.6 | 65.1 | 40.9 | 67.2 | 59.8 | 64.0 |
| 35. | Os03g58330 | 67.7 | 61.9 | 61.3 | 21.3 | 53.9 | 54.2 | 58.7 | 32.7 | 62.6 | 55.9 | 45.4 | 61.6 | 39.2 | 62.2 | 53.8 | 61.9 |
| 36. | Os06g08500 | 43.6 | 43.6 | 45.0 | 23.6 | 45.0 | 48.2 | 47.7 | 28.6 | 42.0 | 43.3 | 51.5 | 44.1 | 46.5 | 44.1 | 45.2 | 46.6 |
| 37. | Os06g09370 | 37.7 | 37.4 | 38.1 | 26.8 | 35.1 | 34.1 | 40.0 | 22.2 | 34.1 | 40.6 | 44.1 | 38.5 | 44.4 | 38.5 | 40.6 | 42.7 |
| 38. | Os02g55250 | 40.6 | 42.0 | 41.5 | 28.5 | 43.5 | 44.4 | 45.1 | 25.2 | 39.2 | 46.0 | 55.8 | 40.8 | 56.3 | 44.0 | 41.7 | 46.3 |
| 39. | Os02g35660 | 35.7 | 35.1 | 36.6 | 29.2 | 36.5 | 34.4 | 39.3 | 22.9 | 33.6 | 38.5 | 43.1 | 34.9 | 45.4 | 36.6 | 39.5 | 37.0 |
| 40. | Os07g08440 | 70.3 | 60.3 | 65.5 | 20.8 | 52.3 | 56.9 | 58.4 | 33.8 | 63.8 | 57.1 | 44.6 | 63.8 | 38.8 | 62.5 | 57.8 | 61.9 |
| 41. | Os09g25040 | 37.3 | 35.7 | 38.5 | 30.0 | 40.1 | 38.5 | 41.5 | 24.4 | 36.1 | 38.7 | 54.3 | 37.7 | 60.7 | 39.3 | 41.5 | 39.3 |
| 42. | Pt_scaff_II.416 | 62.7 | 79.5 | 81.9 | 22.4 | 53.5 | 53.7 | 66.7 | 31.3 | 78.7 | 58.3 | 47.3 | 84.3 | 42.0 | 65.6 | 78.3 | 62.2 |
| 43. | Pt_scaff_70.65 | 60.7 | 62.2 | 65.9 | 21.9 | 52.3 | 49.2 | 65.8 | 27.5 | 60.7 | 69.9 | 44.9 | 60.7 | 40.3 | 71.9 | 64.5 | 74.6 |
| 44. | Pt_scaff_XIII.403 | 59.9 | 62.0 | 62.3 | 21.2 | 50.6 | 49.7 | 66.1 | 28.1 | 58.7 | 67.0 | 45.4 | 59.0 | 42.2 | 69.2 | 65.9 | 73.1 |
| 45. | Pt_scaff28.86 | 39.5 | 39.3 | 40.8 | 27.2 | 39.3 | 38.8 | 42.5 | 29.4 | 36.2 | 43.4 | 45.6 | 39.9 | 48.0 | 40.1 | 42.1 | 42.3 |
| 46. | Pt_TC63334 | 65.0 | 79.8 | 81.6 | 22.1 | 54.8 | 52.3 | 68.7 | 31.3 | 78.7 | 58.0 | 47.6 | 84.3 | 42.6 | 65.0 | 77.2 | 62.5 |
| 47. | TA14134 | 46.2 | 49.8 | 50.8 | 24.7 | 50.5 | 50.2 | 49.3 | 37.5 | 43.7 | 52.5 | 44.6 | 45.2 | 41.2 | 50.2 | 47.1 | 51.4 |
| 48. | TA5414 | 38.0 | 38.5 | 41.2 | 30.1 | 48.5 | 42.7 | 45.9 | 25.9 | 37.8 | 42.9 | 55.8 | 41.2 | 98.9 | 41.2 | 42.1 | 42.7 |
| 49. | TA3263 | 65.3 | 79.5 | 81.6 | 22.3 | 54.8 | 54.0 | 68.4 | 31.7 | 79.0 | 58.3 | 48.3 | 84.7 | 42.6 | 65.0 | 77.2 | 62.8 |
| 50. | TA2825 | 65.2 | 80.1 | 83.9 | 22.0 | 53.2 | 54.2 | 66.7 | 32.1 | 80.3 | 55.9 | 47.3 | 86.0 | 41.6 | 66.6 | 79.5 | 63.7 |
| 51. | TA1616 | 61.0 | 65.3 | 66.3 | 22.3 | 52.1 | 50.3 | 70.4 | 28.8 | 60.4 | 70.4 | 47.8 | 62.9 | 41.6 | 74.2 | 63.9 | 73.4 |
| 52. | TA21665 | 66.2 | 65.2 | 63.9 | 21.3 | 52.3 | 53.2 | 60.4 | 31.1 | 63.5 | 58.3 | 44.4 | 67.6 | 42.9 | 64.4 | 59.5 | 61.0 |
| 53. | TC172581 | 64.5 | 63.8 | 67.1 | 21.1 | 52.9 | 53.9 | 61.0 | 30.6 | 62.2 | 60.0 | 46.6 | 66.1 | 41.8 | 66.3 | 61.3 | 63.4 |
| 54. | AK247217 | 64.6 | 80.5 | 80.3 | 22.9 | 52.3 | 52.0 | 70.1 | 31.5 | 75.2 | 56.2 | 45.1 | 78.1 | 42.0 | 67.8 | 72.5 | 64.4 |
| 55. | TC104646 | 69.5 | 61.6 | 61.9 | 20.7 | 51.0 | 53.5 | 58.7 | 35.0 | 67.3 | 55.7 | 42.0 | 63.3 | 39.2 | 60.3 | 56.1 | 59.8 |
| 56. | DV982110 | 59.3 | 55.6 | 53.5 | 22.6 | 55.8 | 57.9 | 51.0 | 38.0 | 55.8 | 51.3 | 44.9 | 61.6 | 41.6 | 53.4 | 49.1 | 51.7 |
| 57. | WO051050seqID516 | 40.2 | 42.5 | 42.3 | 28.0 | 44.1 | 42.3 | 46.4 | 27.0 | 39.3 | 46.9 | 53.3 | 42.5 | 52.5 | 44.1 | 43.6 | 43.8 |
| 58. | TC68930 | 48.3 | 49.2 | 49.5 | 25.9 | 49.5 | 53.5 | 47.0 | 38.4 | 45.6 | 51.3 | 47.3 | 47.4 | 42.0 | 50.5 | 46.2 | 52.0 |
| 59. | TA30646 | 64.6 | 79.8 | 80.0 | 22.1 | 52.6 | 52.3 | 68.7 | 31.5 | 75.8 | 55.7 | 44.6 | 78.1 | 41.2 | 67.2 | 72.8 | 64.4 |
| 60. | TA28621 | 65.8 | 64.1 | 67.1 | 21.3 | 53.2 | 52.3 | 61.5 | 30.3 | 62.8 | 60.9 | 46.8 | 67.1 | 41.6 | 65.6 | 61.8 | 64.4 |
| 61. | TA34455 | 40.3 | 41.6 | 41.2 | 29.5 | 49.7 | 43.4 | 45.9 | 23.0 | 38.0 | 45.6 | 59.3 | 41.4 | 67.0 | 43.4 | 43.8 | 44.7 |
| 62. | TA37666 | 45.5 | 46.6 | 48.7 | 27.4 | 53.9 | 48.2 | 51.8 | 28.3 | 43.7 | 50.3 | 60.7 | 47.1 | 63.1 | 48.4 | 48.4 | 49.0 |
| 63. | TC253044 | 56.6 | 54.0 | 51.6 | 19.7 | 50.3 | 53.9 | 49.3 | 36.9 | 53.4 | 45.8 | 42.9 | 55.5 | 39.0 | 50.9 | 48.8 | 51.1 |
| 64. | TabHLH11 | 71.2 | 60.9 | 61.0 | 19.9 | 50.6 | 56.6 | 57.0 | 34.2 | 66.2 | 55.9 | 42.7 | 66.2 | 38.8 | 59.7 | 55.8 | 60.1 |
| 65. | TA46156 | 67.5 | 76.8 | 78.4 | 22.3 | 54.8 | 55.2 | 66.4 | 33.6 | 78.9 | 58.6 | 45.9 | 84.1 | 40.5 | 64.7 | 74.6 | 64.0 |
| 66. | GSVIVT00017237001 | 43.5 | 42.1 | 43.1 | 29.4 | 48.1 | 45.2 | 47.4 | 27.5 | 40.4 | 47.8 | 59.1 | 40.4 | 69.5 | 44.0 | 45.5 | 45.7 |
| 67. | TA46194 | 66.1 | 69.0 | 71.9 | 22.3 | 52.1 | 53.4 | 70.9 | 29.7 | 67.4 | 63.5 | 49.0 | 69.0 | 41.8 | 72.5 | 68.8 | 68.3 |
| 68. | GSVIVT00016367001 | 35.7 | 37.7 | 36.5 | 24.5 | 38.1 | 36.9 | 41.6 | 24.1 | 34.4 | 41.9 | 44.5 | 35.5 | 46.2 | 37.7 | 40.8 | 38.6 |
| 69. | TA139285 | 70.2 | 59.9 | 61.3 | 20.3 | 51.6 | 56.2 | 59.5 | 34.4 | 62.1 | 58.0 | 43.4 | 63.2 | 37.5 | 62.5 | 54.6 | 61.9 |
| 70. | TA126400 | 69.5 | 61.9 | 62.6 | 20.9 | 54.5 | 52.5 | 57.3 | 34.0 | 66.7 | 56.8 | 44.9 | 65.3 | 41.4 | 61.6 | 56.1 | 61.0 |
| | | 17 | 18 | 19 | 20 | 21 | 22 | 23 | 24 | 25 | 26 | 27 | 28 | 29 | 30 | 31 | 32 |
| 1. | TC3698 | 52.2 | 51.7 | 51.2 | 34.6 | 51.4 | 53.4 | 51.7 | 31.6 | 48.5 | 38.8 | 52.7 | 25.4 | 54.5 | 53.0 | 53.0 | 32.7 |
| 2. | AT1G03040 | 58.4 | 68.1 | 45.3 | 37.0 | 67.8 | 67.2 | 67.6 | 33.9 | 54.3 | 35.3 | 49.2 | 26.3 | 50.9 | 48.0 | 65.5 | 33.7 |
| 3. | AT4G02590 | 62.6 | 72.6 | 47.0 | 36.4 | 72.3 | 73.6 | 73.3 | 32.8 | 57.1 | 37.7 | 50.3 | 27.5 | 52.6 | 49.5 | 67.5 | 34.3 |
| 4. | AT2G24260 | 15.9 | 16.8 | 15.4 | 17.2 | 16.7 | 16.3 | 16.6 | 22.0 | 15.0 | 21.5 | 16.1 | 15.4 | 16.6 | 16.1 | 16.8 | 23.1 |
| 5. | AT4G30980 | 37.7 | 36.1 | 37.4 | 37.2 | 36.1 | 35.9 | 36.2 | 40.8 | 34.8 | 44.8 | 36.2 | 26.7 | 36.5 | 36.1 | 35.4 | 41.8 |
| 6. | AT5G58010 | 38.8 | 36.0 | 38.1 | 39.2 | 36.0 | 36.4 | 36.3 | 37.2 | 36.9 | 47.3 | 37.7 | 28.1 | 37.8 | 36.9 | 37.9 | 36.0 |
| 7. | TC15501 | 52.4 | 60.9 | 51.0 | 31.4 | 60.7 | 59.0 | 58.0 | 36.2 | 47.9 | 37.1 | 54.0 | 30.8 | 53.8 | 53.8 | 58.7 | 38.2 |
| 8. | TC19278 | 23.0 | 20.2 | 19.4 | 27.6 | 20.2 | 20.2 | 19.6 | 18.4 | 27.3 | 23.1 | 18.0 | 15.6 | 19.0 | 19.3 | 20.5 | 17.9 |
| 9. | TC10015_part | 61.4 | 68.0 | 51.4 | 35.2 | 67.6 | 69.0 | 68.9 | 31.7 | 55.3 | 38.5 | 51.3 | 25.5 | 53.4 | 50.0 | 64.1 | 32.8 |
| 10. | DY268946 | 46.9 | 45.0 | 57.6 | 35.6 | 44.7 | 43.8 | 44.2 | 32.7 | 43.2 | 38.9 | 44.6 | 24.4 | 45.8 | 45.4 | 43.5 | 34.2 |
| 11. | TA2544 | 33.4 | 36.1 | 34.6 | 31.1 | 36.1 | 35.7 | 36.5 | 41.8 | 29.6 | 41.3 | 35.3 | 30.2 | 35.4 | 33.8 | 35.1 | 45.9 |
| 12. | TA3392 | 63.1 | 77.0 | 52.2 | 36.8 | 76.7 | 76.1 | 75.2 | 33.9 | 56.2 | 36.2 | 52.2 | 25.6 | 54.3 | 51.6 | 64.5 | 33.0 |
| 13. | TA12416 | 29.7 | 33.3 | 32.1 | 31.3 | 33.3 | 33.3 | 34.1 | 56.2 | 27.5 | 48.0 | 33.7 | 30.2 | 33.3 | 31.2 | 32.6 | 56.3 |
| 14. | WO051050seqID77 | 50.0 | 54.6 | 58.1 | 31.5 | 54.3 | 54.0 | 52.5 | 33.3 | 47.2 | 36.7 | 54.2 | 27.7 | 54.3 | 56.0 | 53.4 | 34.7 |
| 15. | WO051050seqID1671 | 58.7 | 71.0 | 44.8 | 33.5 | 70.7 | 70.3 | 68.9 | 32.0 | 53.2 | 34.4 | 47.6 | 27.4 | 49.4 | 48.5 | 62.0 | 34.8 |
| 16. | TA55042 | 48.1 | 48.6 | 58.1 | 33.8 | 48.4 | 47.8 | 49.9 | 32.6 | 46.4 | 37.7 | 50.1 | 29.4 | 53.1 | 50.9 | 49.4 | 35.2 |
| 17. | TC207545 | | 64.7 | 56.0 | 37.4 | 64.4 | 65.1 | 65.6 | 30.9 | 77.6 | 37.8 | 52.6 | 23.9 | 55.6 | 52.0 | 61.8 | 32.0 |
| 18. | TA13791 | 71.6 | | 50.6 | 35.4 | 99.7 | 87.2 | 84.9 | 33.7 | 59.7 | 39.4 | 54.0 | 26.2 | 56.7 | 53.3 | 69.0 | 35.6 |
| 19. | CO123623 | 64.9 | 61.7 | | 36.8 | 50.5 | 49.5 | 49.2 | 30.9 | 52.2 | 39.3 | 51.1 | 25.4 | 53.2 | 50.3 | 47.7 | 31.5 |
| 20. | DT543504 | 52.6 | 54.9 | 50.0 | 35.7 | 34.8 | 36.1 | 34.0 | 34.0 | 36.3 | 34.2 | 31.0 | 36.1 | 37.1 | 35.7 | 31.9 | |
| 21. | TC60118 | 71.3 | 99.7 | 60.7 | 55.2 | | 86.8 | 84.6 | 33.7 | 59.4 | 39.1 | 53.7 | 26.2 | 56.4 | 53.0 | 68.7 | 35.6 |
| 22. | TC60119 | 71.1 | 91.1 | 58.6 | 53.6 | 90.8 | | 86.9 | 32.7 | 59.5 | 39.1 | 53.3 | 26.1 | 54.7 | 53.5 | 70.1 | 36.7 |
| 23. | TC61833 | 71.1 | 90.8 | 56.4 | 54.9 | 90.5 | 92.5 | | 33.5 | 59.3 | 37.7 | 52.6 | 26.5 | 55.9 | 53.3 | 71.1 | 36.4 |
| 24. | TC67603 | 38.6 | 42.9 | 40.0 | 41.2 | 42.6 | 42.2 | 42.2 | | 28.5 | 47.1 | 35.0 | 27.3 | 34.9 | 33.2 | 33.2 | 49.6 |
| 25. | TC229602 | 81.1 | 65.3 | 61.9 | 46.1 | 65.0 | 64.5 | 63.9 | 35.8 | | 38.2 | 47.7 | 22.5 | 50.5 | 50.7 | 58.2 | 29.0 |
| 26. | TC205173 | 49.0 | 51.0 | 49.6 | 49.9 | 50.7 | 51.9 | 51.6 | 56.4 | 45.7 | | 39.2 | 25.3 | 37.8 | 37.4 | 39.8 | 47.3 |
| 27. | TA1140 | 61.1 | 66.0 | 62.4 | 53.9 | 65.7 | 66.0 | 65.4 | 45.2 | 54.2 | 52.2 | | 27.1 | 84.2 | 66.0 | 51.2 | 35.8 |
| 28. | TC140470 | 33.5 | 37.8 | 32.8 | 43.9 | 37.6 | 37.4 | 37.8 | 42.8 | 29.7 | 36.4 | 38.0 | | 29.1 | 26.1 | 27.9 | 28.7 |
| 29. | TA3490_part | 63.1 | 68.6 | 63.1 | 52.6 | 68.3 | 66.4 | 66.6 | 44.7 | 57.2 | 49.6 | 87.3 | 37.4 | | 68.0 | 53.2 | 34.4 |
| 30. | CK293938 | 59.8 | 66.0 | 64.9 | 53.6 | 65.7 | 66.8 | 65.6 | 42.2 | 57.4 | 49.6 | 78.1 | 36.2 | 79.4 | | 51.5 | 34.2 |
| 31. | TC8633 | 69.9 | 80.9 | 59.3 | 54.9 | 80.5 | 81.6 | 82.3 | 44.3 | 63.2 | 51.3 | 67.3 | 38.5 | 68.9 | 67.5 | | 36.0 |
| 32. | TC7102 | 38.1 | 44.2 | 39.3 | 41.3 | 44.0 | 44.0 | 45.1 | 62.1 | 34.8 | 55.5 | 44.7 | 43.0 | 42.7 | 44.0 | 44.7 | |
| 33. | TC7103 | 42.9 | 49.1 | 41.6 | 45.5 | 48.8 | 49.1 | 48.3 | 65.8 | 40.1 | 62.5 | 48.8 | 41.0 | 47.5 | 47.0 | 46.5 | 72.9 |
| 34. | TC12771 | 59.7 | 66.0 | 65.8 | 54.9 | 65.7 | 65.8 | 64.6 | 43.1 | 55.7 | 50.7 | 78.1 | 37.4 | 78.5 | 98.0 | 67.5 | 44.5 |
| 35. | Os03g58330 | 59.2 | 63.7 | 62.9 | 51.3 | 63.4 | 63.5 | 60.3 | 43.6 | 56.1 | 50.1 | 64.7 | 36.8 | 67.0 | 66.6 | 66.9 | 42.7 |
| 36. | Os06g08500 | 40.3 | 43.3 | 39.0 | 43.3 | 43.3 | 45.2 | 47.4 | 46.4 | 37.1 | 51.2 | 47.7 | 36.2 | 44.4 | 42.5 | 42.5 | 49.2 |
| 37. | Os06g09370 | 33.1 | 38.9 | 36.2 | 40.8 | 39.1 | 37.4 | 38.1 | 41.4 | 28.7 | 36.6 | 36.4 | 81.5 | 37.2 | 38.7 | 38.3 | 43.9 |
| 38. | Os02g55250 | 38.1 | 41.5 | 39.0 | 43.5 | 41.3 | 41.3 | 42.2 | 56.0 | 35.1 | 50.1 | 42.0 | 45.3 | 44.0 | 41.5 | 44.0 | 60.5 |
| 39. | Os02g35660 | 32.3 | 37.4 | 34.0 | 39.7 | 37.2 | 36.3 | 35.9 | 44.3 | 29.6 | 36.5 | 37.4 | 49.8 | 36.5 | 35.9 | 35.9 | 44.1 |
| 40. | Os07g08440 | 58.4 | 63.7 | 65.9 | 51.0 | 63.4 | 62.8 | 63.9 | 41.0 | 55.3 | 49.6 | 63.4 | 35.6 | 64.5 | 67.6 | 66.6 | 41.8 |
| 41. | Os09g25040 | 33.9 | 38.3 | 35.7 | 37.7 | 38.1 | 38.1 | 38.7 | 54.5 | 31.1 | 47.9 | 38.7 | 42.5 | 36.3 | 36.3 | 37.5 | 58.5 |
| 42. | Pt_scaff_II.416 | 73.3 | 89.4 | 61.0 | 55.2 | 89.1 | 88.2 | 85.6 | 42.4 | 66.0 | 50.4 | 65.0 | 37.0 | 67.3 | 67.0 | 82.1 | 44.5 |
| 43. | Pt_scaff_70.65 | 56.5 | 63.4 | 66.2 | 50.8 | 63.1 | 63.7 | 63.7 | 43.3 | 52.3 | 50.7 | 63.7 | 38.7 | 63.4 | 65.6 | 63.1 | 44.5 |
| 44. | Pt_scaff_XIII.403 | 55.4 | 64.4 | 64.7 | 49.7 | 64.1 | 62.3 | 63.2 | 44.0 | 50.6 | 54.0 | 62.9 | 41.0 | 65.0 | 63.5 | 63.5 | 44.9 |
| 45. | Pt_scaff28.86 | 36.0 | 39.5 | 36.8 | 50.2 | 39.3 | 39.9 | 39.5 | 47.8 | 31.8 | 42.1 | 39.9 | 47.6 | 38.6 | 40.6 | 41.4 | 47.6 |
| 46. | Pt_TC63334 | 73.7 | 89.1 | 62.0 | 53.6 | 88.8 | 88.2 | 86.2 | 42.4 | 66.7 | 51.0 | 63.1 | 37.4 | 67.0 | 64.3 | 82.8 | 45.1 |
| 47. | TA14134 | 45.5 | 50.2 | 46.8 | 50.8 | 50.2 | 48.9 | 46.5 | 43.6 | 42.8 | 54.9 | 50.2 | 35.1 | 49.2 | 48.9 | 51.7 | 43.6 |
| 48. | TA5414 | 37.0 | 42.9 | 39.1 | 39.7 | 42.7 | 41.0 | 41.7 | 67.1 | 33.5 | 55.1 | 42.1 | 48.6 | 41.5 | 42.1 | 41.2 | 69.4 |
| 49. | TA3263 | 73.7 | 89.4 | 62.3 | 54.2 | 89.1 | 88.5 | 86.6 | 43.1 | 66.7 | 51.0 | 63.1 | 37.4 | 67.3 | 64.7 | 82.8 | 45.6 |
| 50. | TA2825 | 74.6 | 90.4 | 60.9 | 55.9 | 90.1 | 90.1 | 89.2 | 43.1 | 67.2 | 51.0 | 66.0 | 35.8 | 67.2 | 66.2 | 83.4 | 46.0 |
| 51. | TA1616 | 57.7 | 66.6 | 68.4 | 54.0 | 66.3 | 65.0 | 65.0 | 42.9 | 55.2 | 51.0 | 65.6 | 38.3 | 63.2 | 65.6 | 66.0 | 47.2 |
| 52. | TA21665 | 60.1 | 66.0 | 62.5 | 52.3 | 65.7 | 66.4 | 66.6 | 42.9 | 54.4 | 51.3 | 73.5 | 36.6 | 76.4 | 84.8 | 64.2 | 43.8 |
| 53. | TC172581 | 59.5 | 67.1 | 62.2 | 54.2 | 66.8 | 67.4 | 65.6 | 42.2 | 55.6 | 50.7 | 76.1 | 38.5 | 76.0 | 92.1 | 66.1 | 43.8 |
| 54. | AK247217 | 70.5 | 82.8 | 60.6 | 52.6 | 82.5 | 84.2 | 84.3 | 43.3 | 63.9 | 49.6 | 67.6 | 38.0 | 67.9 | 69.2 | 95.0 | 42.7 |
| 55. | TC104646 | 63.5 | 63.0 | 65.3 | 49.7 | 62.7 | 61.5 | 61.6 | 43.1 | 59.2 | 49.3 | 62.1 | 35.1 | 66.6 | 67.6 | 62.9 | 40.2 |
| 56. | DV982110 | 56.9 | 58.1 | 62.0 | 56.2 | 57.8 | 57.9 | 57.7 | 44.5 | 56.2 | 58.2 | 58.5 | 35.3 | 57.9 | 55.7 | 55.6 | 43.6 |
| 57. | WO051050seqID516 | 37.8 | 43.2 | 39.5 | 41.9 | 43.0 | 43.6 | 43.6 | 50.5 | 35.0 | 46.0 | 41.9 | 45.9 | 41.3 | 41.9 | 43.2 | 52.9 |
| 58. | TC68930 | 48.3 | 50.2 | 45.0 | 50.5 | 50.5 | 48.0 | 49.5 | 45.2 | 43.5 | 57.9 | 50.2 | 37.0 | 48.9 | 47.1 | 47.4 | 45.1 |
| 59. | TA30646 | 70.2 | 83.5 | 60.6 | 54.2 | 83.2 | 84.2 | 84.3 | 42.9 | 63.9 | 49.6 | 68.3 | 37.6 | 67.5 | 68.5 | 95.4 | 42.2 |
| 60. | TA28621 | 59.5 | 68.1 | 63.5 | 54.2 | 67.8 | 68.4 | 66.6 | 42.4 | 54.9 | 51.3 | 77.1 | 38.7 | 76.6 | 93.8 | 66.8 | 43.6 |
| 61. | TA34455 | 37.1 | 42.5 | 36.9 | 41.2 | 42.3 | 43.4 | 41.8 | 63.1 | 34.2 | 53.0 | 44.5 | 45.3 | 42.3 | 44.3 | 42.7 | 83.2 |
| 62. | TA37666 | 42.1 | 49.0 | 45.3 | 44.5 | 48.7 | 48.7 | 47.4 | 63.7 | 39.8 | 61.0 | 49.5 | 42.4 | 48.2 | 49.5 | 49.5 | 71.1 |
| 63. | TC253044 | 53.4 | 53.1 | 52.8 | 59.8 | 52.8 | 54.9 | 50.8 | 40.3 | 47.6 | 48.4 | 50.3 | 56.5 | 55.9 | 54.7 | 54.0 | 41.3 |
| 64. | TabHLH11 | 61.9 | 62.7 | 66.9 | 50.3 | 62.4 | 62.2 | 61.6 | 40.5 | 58.7 | 50.4 | 63.1 | 35.8 | 66.2 | 66.9 | 65.6 | 41.8 |
| 65. | TA46156 | 74.4 | 85.5 | 64.4 | 56.2 | 85.1 | 85.2 | 83.3 | 44.7 | 67.8 | 53.4 | 67.3 | 38.0 | 70.0 | 70.9 | 81.1 | 43.3 |
| 66. | GSVIVT00017237001 | 40.9 | 45.9 | 39.7 | 43.8 | 45.9 | 45.0 | 45.9 | 64.9 | 35.9 | 61.2 | 44.0 | 42.6 | 42.1 | 42.8 | 46.2 | 68.2 |
| 67. | TA46194 | 64.5 | 73.5 | 66.8 | 53.4 | 73.2 | 72.5 | 72.2 | 44.3 | 57.8 | 54.3 | 70.6 | 38.7 | 72.8 | 74.8 | 73.2 | 44.9 |
| 68. | GSVIVT00016367001 | 32.8 | 37.1 | 34.6 | 45.4 | 36.9 | 36.5 | 38.1 | 44.3 | 29.9 | 41.6 | 39.0 | 50.3 | 36.9 | 35.3 | 38.8 | 45.4 |
| 69. | TA139285 | 60.7 | 61.1 | 66.0 | 48.0 | 60.7 | 61.5 | 61.0 | 40.7 | 56.1 | 49.0 | 64.7 | 35.6 | 66.2 | 68.9 | 64.2 | 41.1 |
| 70. | TA126400 | 62.5 | 63.0 | 63.2 | 50.0 | 62.7 | 61.8 | 63.6 | 42.6 | 57.9 | 49.3 | 62.4 | 34.9 | 65.9 | 66.9 | 62.3 | 40.0 |
| | | 33 | 34 | 35 | 36 | 37 | 38 | 39 | 40 | 41 | 42 | 43 | 44 | 45 | 46 | 47 | 48 |
| 1. | TC3698 | 36.2 | 53.3 | 56.0 | 31.1 | 26.5 | 32.4 | 26.5 | 56.9 | 30.0 | 50.8 | 48.7 | 47.8 | 28.7 | 50.8 | 31.8 | 29.7 |
| 2. | AT1G03040 | 35.4 | 48.3 | 51.4 | 30.9 | 25.8 | 34.1 | 27.9 | 49.8 | 29.5 | 65.7 | 48.6 | 47.5 | 29.9 | 66.7 | 31.1 | 31.1 |
| 3. | AT4G02590 | 35.4 | 50.8 | 52.7 | 32.8 | 27.8 | 33.6 | 28.7 | 51.2 | 31.7 | 71.8 | 52.1 | 49.3 | 30.9 | 72.8 | 31.6 | 33.6 |
| 4. | AT2G24260 | 21.8 | 16.3 | 16.7 | 17.5 | 15.5 | 21.2 | 16.4 | 16.4 | 21.4 | 16.7 | 16.6 | 15.5 | 18.1 | 17.0 | 18.9 | 23.0 |
| 5. | AT4G30980 | 44.3 | 35.6 | 39.8 | 33.9 | 25.5 | 36.2 | 28.6 | 37.6 | 33.8 | 35.5 | 36.1 | 33.6 | 29.6 | 36.4 | 34.9 | 41.6 |
| 6. | AT5G58010 | 39.7 | 36.4 | 37.4 | 37.7 | 26.9 | 36.4 | 26.1 | 37.4 | 31.9 | 34.6 | 34.2 | 34.1 | 31.3 | 35.8 | 36.3 | 35.6 |
| 7. | TC15501 | 37.6 | 53.0 | 52.5 | 32.7 | 29.4 | 35.0 | 30.7 | 52.0 | 33.5 | 59.7 | 55.6 | 55.5 | 30.7 | 62.0 | 30.8 | 35.2 |
| 8. | TC19278 | 20.2 | 18.6 | 19.5 | 20.8 | 15.6 | 19.1 | 17.9 | 19.8 | 17.0 | 19.7 | 17.0 | 18.0 | 22.7 | 19.7 | 29.4 | 19.0 |
| 9. | TC10015_part | 35.7 | 50.6 | 51.8 | 32.9 | 26.0 | 32.9 | 26.7 | 52.8 | 30.6 | 67.5 | 50.4 | 48.7 | 27.4 | 68.5 | 30.9 | 31.0 |
| 10. | DY268946 | 33.5 | 45.1 | 46.9 | 31.6 | 26.5 | 32.9 | 26.6 | 47.9 | 31.5 | 42.9 | 54.2 | 51.6 | 29.6 | 44.0 | 36.8 | 33.1 |
| 11. | TA2544 | 42.6 | 35.0 | 35.1 | 39.3 | 31.1 | 42.5 | 32.1 | 35.3 | 43.3 | 36.2 | 36.9 | 36.4 | 33.1 | 36.4 | 32.5 | 45.2 |
| 12. | TA3392 | 35.2 | 52.1 | 53.0 | 30.4 | 28.9 | 32.7 | 26.2 | 51.5 | 30.8 | 76.3 | 49.3 | 48.6 | 28.3 | 78.0 | 30.2 | 31.0 |
| 13. | TA12416 | 55.6 | 31.8 | 32.2 | 36.9 | 28.7 | 45.2 | 30.6 | 31.4 | 47.5 | 32.7 | 32.7 | 33.3 | 32.5 | 33.5 | 31.2 | 98.3 |
| 14. | WO051050seqID77 | 35.6 | 55.9 | 52.0 | 31.9 | 28.8 | 34.5 | 27.4 | 51.6 | 32.3 | 54.0 | 63.0 | 61.3 | 29.3 | 54.6 | 32.1 | 32.0 |
| 15. | WO051050seqID1671 | 35.0 | 48.7 | 48.7 | 32.1 | 28.7 | 33.1 | 29.3 | 48.6 | 33.4 | 70.4 | 50.5 | 50.7 | 30.9 | 70.3 | 28.9 | 32.9 |
| 16. | TA55042 | 36.8 | 50.3 | 51.3 | 33.9 | 32.2 | 36.3 | 27.3 | 51.0 | 31.1 | 47.8 | 61.3 | 60.1 | 30.4 | 48.1 | 30.9 | 33.3 |
| 17. | TC207545 | 32.9 | 53.0 | 54.1 | 28.6 | 23.9 | 31.3 | 24.9 | 53.2 | 28.8 | 67.3 | 48.3 | 44.8 | 27.2 | 68.3 | 32.8 | 30.2 |
| 18. | TA13791 | 38.5 | 53.3 | 54.6 | 33.2 | 27.1 | 33.8 | 28.2 | 51.7 | 32.5 | 80.7 | 50.1 | 50.7 | 30.7 | 82.0 | 32.6 | 33.3 |
| 19. | CO123623 | 33.6 | 52.0 | 50.6 | 31.1 | 29.0 | 30.1 | 27.3 | 50.9 | 29.6 | 50.3 | 58.4 | 56.5 | 26.2 | 51.5 | 32.1 | 33.1 |
| 20. | DT543504 | 33.4 | 38.1 | 34.5 | 31.8 | 28.8 | 32.3 | 30.8 | 32.5 | 29.0 | 33.3 | 33.3 | 32.7 | 40.8 | 32.9 | 36.1 | 30.8 |
| 21. | TC60118 | 38.5 | 53.0 | 54.3 | 33.2 | 27.5 | 33.6 | 28.2 | 51.4 | 32.3 | 80.4 | 49.9 | 50.4 | 30.7 | 81.7 | 32.6 | 33.3 |
| 22. | TC60119 | 37.3 | 54.4 | 53.1 | 33.1 | 27.0 | 34.2 | 28.2 | 51.1 | 31.4 | 80.5 | 49.9 | 48.6 | 30.1 | 81.8 | 31.2 | 33.6 |
| 23. | TC61833 | 36.8 | 53.9 | 51.1 | 34.5 | 25.4 | 34.7 | 27.4 | 52.0 | 31.2 | 78.9 | 49.1 | 48.4 | 30.1 | 80.5 | 30.2 | 34.7 |
| 24. | TC67603 | 52.5 | 33.7 | 34.3 | 35.6 | 28.3 | 40.6 | 30.7 | 32.6 | 43.3 | 33.3 | 33.2 | 32.0 | 33.3 | 34.0 | 31.5 | 56.8 |
| 25. | TC229602 | 32.3 | 50.0 | 50.8 | 28.2 | 21.3 | 28.4 | 22.7 | 49.3 | 26.2 | 61.0 | 43.8 | 41.6 | 25.1 | 62.3 | 32.9 | 27.7 |
| 26. | TC205173 | 53.1 | 38.0 | 38.7 | 40.3 | 26.6 | 41.2 | 27.9 | 38.6 | 41.4 | 38.3 | 36.5 | 36.4 | 30.9 | 38.3 | 36.8 | 47.9 |
| 27. | TA1140 | 37.5 | 67.0 | 52.5 | 33.3 | 27.2 | 31.8 | 28.2 | 52.1 | 31.2 | 54.7 | 54.0 | 50.8 | 27.7 | 52.7 | 31.9 | 34.3 |
| 28. | TC140470 | 27.7 | 27.0 | 26.6 | 24.4 | 71.1 | 28.1 | 35.1 | 26.1 | 28.2 | 26.2 | 28.1 | 28.7 | 31.9 | 25.2 | 24.0 | 31.2 |
| 29. | TA3490_part | 36.5 | 68.5 | 54.0 | 32.7 | 28.3 | 33.3 | 27.6 | 53.7 | 30.3 | 54.9 | 53.0 | 52.6 | 29.0 | 54.3 | 31.7 | 33.3 |
| 30. | CK293938 | 37.5 | 96.3 | 53.8 | 32.4 | 28.0 | 33.9 | 27.5 | 53.5 | 30.0 | 53.0 | 52.7 | 50.9 | 28.6 | 52.0 | 30.9 | 31.2 |
| 31. | TC8633 | 34.9 | 52.5 | 53.8 | 33.0 | 29.2 | 33.6 | 26.9 | 52.6 | 31.4 | 69.9 | 52.7 | 50.7 | 30.5 | 71.2 | 34.0 | 32.1 |
| 32. | TC7102 | 64.6 | 34.5 | 35.9 | 37.5 | 28.8 | 47.3 | 32.6 | 34.1 | 48.4 | 35.3 | 33.5 | 34.4 | 32.9 | 35.7 | 32.8 | 56.1 |
| 33. | TC7103 | | 38.2 | 37.8 | 41.1 | 28.9 | 46.2 | 29.6 | 38.3 | 44.7 | 34.3 | 34.8 | 36.7 | 32.9 | 35.7 | 34.1 | 55.3 |
| 34. | TC12771 | 48.1 | | 54.6 | 32.2 | 28.6 | 34.9 | 26.9 | 54.7 | 30.0 | 53.6 | 53.0 | 50.6 | 28.8 | 51.7 | 31.4 | 31.9 |
| 35. | Os03g58330 | 46.0 | 66.1 | | 36.6 | 27.1 | 35.3 | 28.0 | 74.9 | 32.3 | 54.3 | 50.1 | 49.0 | 27.4 | 51.2 | 33.5 | 31.8 |
| 36. | Os06g08500 | 55.6 | 45.0 | 47.4 | | 24.0 | 46.8 | 29.1 | 36.2 | 38.0 | 32.9 | 32.1 | 31.3 | 27.7 | 34.3 | 32.3 | 37.9 |
| 37. | Os06g09370 | 41.2 | 38.5 | 35.8 | 37.4 | | 28.6 | 34.3 | 26.6 | 28.6 | 27.6 | 31.2 | 30.4 | 32.1 | 27.9 | 23.9 | 27.8 |
| 38. | Os02g55250 | 59.0 | 43.5 | 42.2 | 59.2 | 46.4 | | 31.8 | 35.3 | 46.4 | 33.4 | 34.2 | 34.4 | 32.8 | 33.7 | 33.5 | 45.8 |
| 39. | Os02g35660 | 40.3 | 36.5 | 36.1 | 36.6 | 47.9 | 45.6 | | 25.7 | 30.4 | 26.4 | 29.1 | 27.2 | 31.9 | 26.1 | 25.7 | 30.5 |
| 40. | Os07g08440 | 45.7 | 68.1 | 82.0 | 46.0 | 35.8 | 41.7 | 34.0 | 31.4 | 53.4 | 50.0 | 47.8 | 27.7 | 52.3 | 31.7 | 31.7 | |
| 41. | Os09g25040 | 54.9 | 37.1 | 37.3 | 47.3 | 43.7 | 56.3 | 44.8 | 37.5 | | 30.6 | 31.2 | 31.2 | 31.1 | 30.9 | 29.1 | 47.7 |
| 42. | Pt_scaff_II.416 | 48.3 | 66.7 | 63.3 | 44.7 | 39.5 | 43.3 | 36.6 | 64.3 | 38.3 | | 50.3 | 49.6 | 29.1 | 93.7 | 30.5 | 33.0 |
| 43. | Pt_scaff_70.65 | 47.0 | 65.9 | 60.7 | 44.7 | 42.9 | 44.2 | 37.8 | 60.7 | 38.1 | 63.1 | | 88.4 | 29.5 | 50.0 | 31.0 | 32.4 |
| 44. | Pt_scaff_XIII.403 | 47.5 | 63.2 | 59.9 | 43.6 | 41.8 | 44.9 | 38.2 | 59.0 | 38.5 | 63.2 | 90.7 | | 27.9 | 49.6 | 28.2 | 32.3 |
| 45. | Pt_scaff28.86 | 45.8 | 40.1 | 36.4 | 41.2 | 49.6 | 47.6 | 45.6 | 35.3 | 46.1 | 39.3 | 40.4 | 40.6 | | 29.8 | 31.8 | 33.2 |
| 46. | Pt_TC63334 | 48.8 | 64.7 | 64.0 | 44.1 | 38.3 | 43.5 | 35.3 | 65.0 | 38.1 | 95.3 | 63.4 | 61.4 | 40.4 | | 30.8 | 33.6 |
| 47. | TA14134 | 45.5 | 49.8 | 47.7 | 46.6 | 36.8 | 44.7 | 36.8 | 46.8 | 38.1 | 48.3 | 49.2 | 47.6 | 42.1 | 49.2 | | 30.1 |
| 48. | TA5414 | 63.9 | 40.8 | 38.5 | 47.2 | 44.8 | 57.1 | 45.4 | 39.3 | 60.5 | 42.1 | 41.0 | 42.1 | 47.9 | 42.7 | 39.7 | |
| 49. | TA3263 | 49.1 | 65.0 | 64.0 | 44.4 | 38.7 | 43.8 | 35.1 | 64.3 | 38.7 | 95.0 | 63.4 | 61.4 | 41.0 | 99.0 | 49.8 | 42.1 |
| 50. | TA2825 | 49.4 | 66.2 | 63.2 | 45.0 | 37.9 | 43.3 | 36.6 | 65.2 | 39.1 | 93.3 | 64.7 | 62.9 | 39.0 | 93.0 | 50.2 | 41.2 |
| 51. | TA1616 | 47.8 | 66.6 | 63.2 | 48.5 | 40.8 | 46.3 | 36.3 | 66.0 | 39.5 | 65.6 | 83.7 | 80.5 | 41.2 | 64.1 | 50.3 | 40.6 |
| 52. | TA21665 | 48.6 | 84.9 | 65.9 | 44.7 | 35.6 | 43.3 | 37.2 | 65.9 | 37.7 | 67.0 | 63.7 | 63.2 | 37.7 | 66.7 | 48.0 | 41.2 |
| 53. | TC172581 | 48.3 | 92.8 | 65.8 | 44.4 | 38.5 | 41.5 | 36.6 | 64.5 | 38.1 | 68.4 | 64.7 | 63.5 | 39.9 | 66.8 | 49.8 | 42.1 |
| 54. | AK247217 | 47.3 | 67.5 | 64.6 | 43.6 | 37.9 | 44.2 | 35.9 | 64.6 | 37.9 | 83.8 | 65.3 | 63.8 | 42.8 | 84.4 | 50.8 | 41.9 |
| 55. | TC104646 | 44.2 | 67.1 | 77.6 | 45.2 | 35.8 | 40.1 | 34.0 | 85.0 | 37.1 | 63.3 | 57.4 | 57.8 | 36.0 | 63.7 | 44.6 | 38.9 |
| 56. | DV982110 | 50.4 | 54.4 | 57.5 | 46.0 | 35.6 | 43.5 | 34.9 | 59.0 | 38.9 | 58.3 | 54.1 | 50.3 | 40.1 | 58.3 | 51.1 | 41.7 |
| 57. | WO051050seqID516 | 48.8 | 42.1 | 42.5 | 44.3 | 44.6 | 51.6 | 42.6 | 41.9 | 52.3 | 44.7 | 44.5 | 44.7 | 49.5 | 42.5 | 42.8 | 51.7 |
| 58. | TC68930 | 50.4 | 47.1 | 49.8 | 47.7 | 37.0 | 45.8 | 35.9 | 46.8 | 40.9 | 49.8 | 48.0 | 46.1 | 45.0 | 50.8 | 63.7 | 40.4 |
| 59. | TA30646 | 46.8 | 68.5 | 62.3 | 43.1 | 39.3 | 45.6 | 36.3 | 65.9 | 37.9 | 84.1 | 65.3 | 63.8 | 42.8 | 84.8 | 49.8 | 41.7 |
| 60. | TA28621 | 48.6 | 94.4 | 66.8 | 45.0 | 38.5 | 41.5 | 36.5 | 64.1 | 37.7 | 68.8 | 65.3 | 63.8 | 40.4 | 67.4 | 48.3 | 41.2 |
| 61. | TA34455 | 71.4 | 43.6 | 44.3 | 47.4 | 44.4 | 60.0 | 43.1 | 41.6 | 59.5 | 42.5 | 44.7 | 46.5 | 46.1 | 41.2 | 42.7 | 66.9 |
| 62. | TA37666 | 88.9 | 50.3 | 48.2 | 54.7 | 41.8 | 55.8 | 38.5 | 47.6 | 52.3 | 49.7 | 51.0 | 49.2 | 43.9 | 50.0 | 47.1 | 63.5 |
| 63. | TC253044 | 45.7 | 55.7 | 52.7 | 42.5 | 50.0 | 40.6 | 38.4 | 52.9 | 36.5 | 54.0 | 49.8 | 50.3 | 41.4 | 53.3 | 46.8 | 39.1 |
| 64. | TabHLH11 | 45.2 | 67.8 | 86.4 | 43.3 | 36.8 | 41.0 | 36.3 | 80.5 | 36.5 | 62.3 | 58.3 | 59.0 | 36.4 | 63.0 | 45.5 | 38.2 |
| 65. | TA46156 | 48.3 | 70.8 | 66.0 | 44.1 | 37.0 | 42.4 | 35.7 | 66.2 | 37.5 | 85.0 | 64.7 | 61.4 | 39.0 | 85.3 | 50.5 | 40.2 |
| 66. | GSVIVT00017237001 | 65.1 | 43.8 | 43.3 | 52.4 | 43.1 | 58.5 | 42.4 | 44.7 | 56.5 | 44.3 | 45.9 | 48.1 | 47.6 | 45.0 | 44.0 | 69.2 |
| 67. | TA46194 | 49.6 | 75.1 | 68.4 | 45.8 | 39.1 | 45.1 | 37.6 | 68.7 | 39.7 | 73.8 | 70.4 | 69.2 | 40.1 | 73.5 | 49.5 | 42.1 |
| 68. | GSVIVT00016367001 | 44.3 | 35.5 | 36.5 | 39.6 | 50.5 | 46.2 | 49.4 | 35.9 | 45.9 | 38.8 | 40.6 | 41.2 | 63.9 | 36.7 | 39.0 | 45.6 |
| 69. | TA139285 | 46.8 | 68.1 | 90.5 | 45.5 | 38.1 | 40.4 | 34.0 | 84.0 | 37.7 | 61.7 | 58.3 | 58.4 | 38.4 | 61.7 | 47.1 | 38.2 |
| 70. | TA126400 | 45.2 | 66.1 | 76.2 | 44.1 | 35.6 | 40.8 | 35.3 | 82.9 | 36.7 | 64.0 | 58.0 | 55.7 | 36.4 | 64.7 | 45.8 | 41.5 |

| | | 49 | 50 | 51 | 52 | 53 | 54 | 55 | 56 | 57 | 58 | 59 | 60 | 61 | 62 | 63 |
|---|---|---|---|---|---|---|---|---|---|---|---|---|---|---|---|---|
| 1. | TC3698 | 51.4 | 54.0 | 50.6 | 52.3 | 50.2 | 54.4 | 58.0 | 45.1 | 33.3 | 33.4 | 54.7 | 50.8 | 31.4 | 35.3 | 37.5 |
| 2. | AT1G03040 | 66.7 | 70.0 | 52.4 | 46.8 | 49.2 | 66.5 | 50.6 | 40.2 | 33.0 | 31.8 | 67.0 | 49.2 | 32.8 | 36.4 | 37.3 |
| 3. | AT4G02590 | 73.1 | 75.2 | 54.0 | 49.4 | 53.2 | 70.2 | 53.4 | 43.1 | 33.3 | 32.5 | 69.9 | 52.7 | 33.4 | 38.9 | 37.0 |
| 4. | AT2G24260 | 17.0 | 16.4 | 17.3 | 15.6 | 15.4 | 17.0 | 16.6 | 18.5 | 20.8 | 19.9 | 16.5 | 15.7 | 20.6 | 21.8 | 14.8 |
| 5. | AT4G30980 | 36.8 | 37.5 | 36.4 | 36.1 | 36.9 | 36.4 | 41.0 | 42.2 | 34.9 | 36.4 | 36.6 | 35.7 | 41.3 | 43.2 | 35.3 |
| 6. | AT5G58010 | 35.8 | 35.1 | 33.9 | 36.2 | 35.1 | 35.4 | 38.3 | 46.5 | 33.5 | 39.9 | 36.2 | 34.7 | 36.6 | 40.5 | 36.6 |
| 7. | TC15501 | 61.5 | 61.2 | 59.3 | 51.0 | 51.4 | 61.1 | 51.6 | 41.5 | 36.4 | 30.0 | 60.3 | 51.7 | 37.0 | 37.5 | 34.0 |
| 8. | TC19278 | 20.5 | 20.3 | 18.3 | 18.8 | 18.4 | 19.8 | 21.0 | 25.1 | 20.2 | 30.4 | 20.1 | 18.4 | 17.0 | 20.6 | 24.1 |
| 9. | TC10015_part | 68.8 | 70.7 | 53.3 | 48.7 | 50.0 | 65.9 | 51.8 | 42.4 | 32.8 | 31.8 | 66.2 | 50.0 | 32.7 | 35.1 | 36.8 |
| 10. | DY268946 | 44.0 | 43.7 | 57.3 | 42.4 | 43.9 | 41.7 | 47.0 | 40.7 | 35.5 | 35.9 | 44.5 | 44.9 | 34.0 | 34.6 | 33.1 |
| 11. | TA2544 | 36.6 | 36.1 | 37.0 | 33.5 | 35.9 | 35.7 | 35.1 | 37.6 | 40.8 | 34.2 | 34.5 | 36.1 | 46.6 | 45.3 | 31.7 |
| 12. | TA3392 | 78.0 | 76.7 | 52.6 | 53.2 | 52.5 | 68.3 | 51.8 | 42.8 | 33.4 | 31.3 | 68.0 | 53.2 | 34.1 | 36.5 | 37.5 |
| 13. | TA12416 | 32.8 | 33.5 | 33.5 | 30.3 | 32.2 | 32.6 | 32.9 | 35.2 | 37.7 | 31.7 | 32.2 | 32.2 | 53.7 | 52.9 | 28.8 |
| 14. | WO051050seqID77 | 54.6 | 54.3 | 63.7 | 54.0 | 54.0 | 55.3 | 52.0 | 41.4 | 34.8 | 31.2 | 55.0 | 53.5 | 35.8 | 33.9 | 36.7 |
| 15. | WO051050seqID1671 | 70.6 | 73.4 | 51.6 | 46.5 | 48.3 | 65.7 | 48.6 | 38.5 | 33.5 | 29.4 | 66.0 | 48.6 | 33.8 | 34.8 | 33.8 |
| 16. | TA55042 | 48.1 | 50.0 | 62.5 | 47.4 | 50.3 | 49.2 | 51.5 | 40.4 | 31.6 | 33.0 | 49.5 | 50.9 | 35.3 | 36.8 | 35.1 |
| 17. | TC207545 | 68.7 | 69.8 | 48.5 | 50.7 | 51.6 | 64.1 | 56.7 | 49.3 | 31.8 | 35.2 | 64.4 | 51.3 | 29.6 | 32.8 | 39.7 |
| 18. | TA13791 | 82.7 | 82.1 | 54.9 | 53.4 | 54.2 | 71.8 | 54.9 | 43.3 | 35.4 | 32.8 | 72.4 | 54.2 | 36.0 | 38.0 | 36.3 |
| 19. | CO123623 | 51.5 | 48.1 | 61.3 | 47.6 | 48.1 | 45.8 | 53.4 | 47.8 | 32.2 | 33.1 | 46.1 | 49.7 | 29.8 | 33.3 | 36.7 |
| 20. | DT543504 | 33.5 | 35.1 | 33.1 | 35.0 | 36.4 | 34.1 | 34.6 | 41.2 | 31.0 | 37.2 | 35.3 | 37.2 | 32.0 | 33.9 | 41.4 |
| 21. | TC60118 | 82.4 | 81.8 | 54.7 | 53.1 | 53.9 | 71.5 | 54.6 | 43.0 | 35.2 | 32.3 | 72.1 | 53.9 | 36.0 | 38.0 | 36.3 |
| 22. | TC60119 | 82.4 | 82.8 | 53.3 | 53.8 | 54.1 | 73.5 | 51.7 | 42.9 | 35.1 | 32.4 | 73.8 | 55.0 | 34.8 | 37.5 | 36.4 |
| 23. | TC61833 | 81.2 | 81.6 | 53.3 | 53.1 | 55.0 | 74.3 | 52.3 | 43.2 | 35.2 | 33.3 | 74.3 | 54.9 | 34.0 | 36.7 | 34.1 |
| 24. | TC67603 | 33.9 | 32.8 | 34.2 | 32.5 | 33.0 | 32.7 | 33.9 | 37.6 | 36.3 | 33.2 | 32.5 | 33.6 | 46.9 | 51.7 | 30.2 |
| 25. | TC229602 | 62.7 | 64.2 | 47.9 | 47.0 | 49.0 | 59.8 | 52.5 | 48.2 | 29.4 | 34.5 | 60.1 | 49.0 | 27.7 | 31.3 | 34.7 |
| 26. | TC205173 | 38.3 | 39.6 | 36.5 | 37.0 | 38.1 | 38.2 | 38.2 | 48.5 | 36.9 | 40.7 | 38.0 | 37.3 | 42.1 | 48.7 | 35.1 |
| 27. | TA1140 | 52.7 | 52.9 | 53.7 | 61.7 | 64.3 | 55.4 | 52.8 | 41.1 | 31.4 | 32.8 | 54.0 | 64.9 | 34.5 | 37.4 | 36.7 |
| 28. | TC140470 | 25.3 | 24.9 | 27.4 | 27.6 | 27.4 | 29.0 | 26.1 | 27.3 | 27.7 | 25.0 | 28.6 | 27.4 | 30.3 | 28.1 | 55.1 |
| 29. | TA3490_part | 54.9 | 55.0 | 53.7 | 66.0 | 67.0 | 55.5 | 56.8 | 45.3 | 32.3 | 33.6 | 55.5 | 67.3 | 33.6 | 36.7 | 36.5 |
| 30. | CK293938 | 52.6 | 54.0 | 54.1 | 76.4 | 87.9 | 53.3 | 56.8 | 41.2 | 32.7 | 32.7 | 53.5 | 88.9 | 34.3 | 37.7 | 36.6 |
| 31. | TC8633 | 71.5 | 72.4 | 52.6 | 51.5 | 51.9 | 90.8 | 52.7 | 43.9 | 35.0 | 31.9 | 91.1 | 51.9 | 33.7 | 36.8 | 35.2 |
| 32. | TC7102 | 35.7 | 36.7 | 36.8 | 33.2 | 35.6 | 35.7 | 34.3 | 36.2 | 38.0 | 33.4 | 35.2 | 35.3 | 71.8 | 63.0 | 31.4 |
| 33. | TC7103 | 36.2 | 37.2 | 37.7 | 36.6 | 37.8 | 36.2 | 37.0 | 41.0 | 37.6 | 37.0 | 36.0 | 38.5 | 62.4 | 83.3 | 33.6 |
| 34. | TC12771 | 52.3 | 54.0 | 54.7 | 77.7 | 89.2 | 54.1 | 57.4 | 40.9 | 32.1 | 33.1 | 53.8 | 90.5 | 34.6 | 38.1 | 36.6 |
| 35. | Os03g58330 | 51.2 | 55.0 | 52.8 | 51.1 | 52.1 | 53.6 | 72.9 | 44.2 | 34.1 | 34.6 | 54.0 | 52.7 | 36.6 | 37.6 | 34.9 |
| 36. | Os06g08500 | 34.6 | 33.9 | 34.5 | 29.6 | 33.1 | 33.1 | 33.3 | 36.6 | 32.8 | 32.0 | 32.8 | 32.7 | 39.0 | 40.0 | 30.1 |
| 37. | Os06g09370 | 28.1 | 26.7 | 30.1 | 26.3 | 28.6 | 28.5 | 27.4 | 28.2 | 27.5 | 25.4 | 28.4 | 27.6 | 30.3 | 28.4 | 45.0 |
| 38. | Os02g55250 | 34.7 | 34.1 | 35.8 | 33.3 | 34.2 | 33.5 | 34.0 | 36.1 | 36.8 | 34.3 | 34.6 | 34.0 | 46.1 | 44.2 | 31.3 |
| 39. | Os02g35660 | 26.4 | 28.2 | 27.7 | 25.5 | 27.8 | 27.3 | 26.5 | 27.9 | 29.1 | 26.6 | 27.6 | 28.1 | 31.3 | 29.8 | 29.5 |
| 40. | Os07g08440 | 52.3 | 56.2 | 55.8 | 51.9 | 50.8 | 51.4 | 81.3 | 45.6 | 33.6 | 31.9 | 52.6 | 50.8 | 33.6 | 36.3 | 35.3 |
| 41. | Os09g25040 | 31.7 | 31.5 | 31.6 | 29.4 | 30.5 | 31.7 | 31.1 | 32.5 | 37.2 | 31.3 | 31.5 | 30.4 | 45.6 | 43.6 | 27.9 |
| 42. | Pt_scaff_II.416 | 93.0 | 88.9 | 52.1 | 52.5 | 54.9 | 72.7 | 53.4 | 44.4 | 36.5 | 32.4 | 73.0 | 54.5 | 34.1 | 35.6 | 35.5 |
| 43. | Pt_scaff_70.65 | 49.7 | 51.6 | 73.2 | 51.5 | 51.7 | 52.3 | 49.4 | 42.3 | 34.6 | 31.6 | 52.0 | 52.0 | 34.1 | 37.6 | 35.0 |
| 44. | Pt_scaff_XIII.403 | 49.3 | 49.6 | 71.3 | 49.9 | 50.4 | 50.6 | 48.8 | 40.0 | 34.8 | 30.5 | 50.6 | 51.3 | 34.7 | 36.5 | 34.2 |
| 45. | Pt_scaff28.86 | 30.3 | 28.9 | 29.8 | 28.7 | 27.9 | 30.1 | 27.6 | 31.1 | 33.6 | 32.2 | 30.1 | 28.5 | 32.2 | 32.3 | 31.2 |
| 46. | Pt_TC63334 | 98.7 | 89.2 | 52.3 | 52.5 | 53.9 | 74.0 | 53.7 | 44.6 | 35.2 | 33.1 | 74.3 | 53.9 | 33.9 | 37.2 | 34.8 |
| 47. | TA14134 | 30.8 | 31.4 | 31.4 | 29.6 | 30.5 | 33.2 | 31.4 | 39.1 | 33.7 | 49.4 | 33.4 | 30.0 | 30.4 | 35.1 | 32.5 |
| 48. | TA5414 | 32.7 | 33.8 | 32.4 | 30.3 | 32.5 | 32.4 | 31.9 | 35.3 | 38.1 | 30.2 | 31.8 | 32.5 | 52.7 | 53.3 | 29.2 |
| 49. | TA3263 | | 89.2 | 52.6 | 53.1 | 54.5 | 74.3 | 54.3 | 44.9 | 35.8 | 33.6 | 74.6 | 54.5 | 33.8 | 37.7 | 34.8 |
| 50. | TA2825 | 93.3 | | 53.6 | 53.6 | 54.9 | 76.3 | 54.0 | 44.8 | 35.0 | 32.0 | 76.3 | 54.9 | 34.6 | 35.8 | 35.6 |
| 51. | TA1616 | 64.1 | 66.3 | | 51.1 | 52.4 | 54.1 | 54.8 | 42.5 | 35.7 | 32.1 | 54.1 | 53.6 | 35.0 | 36.8 | 34.7 |
| 52. | TA21665 | 67.0 | 66.6 | 65.0 | | 75.2 | 52.3 | 53.8 | 43.0 | 32.0 | 32.5 | 52.0 | 76.5 | 32.3 | 36.3 | 35.6 |
| 53. | TC172581 | 67.1 | 68.4 | 65.6 | 82.2 | | 53.3 | 54.0 | 43.5 | 34.0 | 31.8 | 52.1 | 96.7 | 34.2 | 37.7 | 37.1 |
| 54. | AK247217 | 84.4 | 86.1 | 66.9 | 65.6 | 66.8 | | 52.8 | 44.3 | 35.0 | 32.8 | 98.3 | 53.8 | 34.2 | 36.4 | 34.4 |
| 55. | TC104646 | 64.0 | 62.9 | 61.7 | 65.9 | 65.8 | 62.9 | | 48.5 | 34.2 | 34.4 | 53.5 | 55.0 | 34.4 | 36.3 | 35.7 |
| 56. | DV982110 | 58.7 | 57.5 | 53.7 | 57.4 | 53.3 | 55.0 | 60.3 | | 54.2 | 40.7 | 45.0 | 43.2 | 34.7 | 38.2 | 41.9 |
| 57. | WO051050seqID516 | 43.0 | 43.0 | 46.0 | 41.7 | 43.0 | 42.3 | 42.3 | 55.7 | | 37.2 | 34.6 | 33.7 | 38.0 | 37.3 | 29.6 |
| 58. | TC68930 | 51.1 | 48.9 | 50.2 | 48.3 | 50.2 | 49.5 | 47.7 | 53.2 | 47.9 | | 32.9 | 31.7 | 31.5 | 36.6 | 35.7 |
| 59. | TA30646 | 84.8 | 85.8 | 66.9 | 65.2 | 66.8 | 99.0 | 63.6 | 56.6 | 42.1 | 49.2 | | 53.0 | 32.0 | 36.7 | 34.4 |
| 60. | TA28621 | 67.8 | 69.1 | 66.0 | 83.6 | 97.7 | 67.1 | 65.5 | 53.6 | 43.8 | 48.3 | 67.4 | 33.6 | 39.1 | 37.4 | 51.4 |
| 61. | TA34455 | 40.9 | 42.5 | 43.0 | 40.9 | 43.2 | 41.4 | 40.0 | 42.5 | 53.8 | 44.5 | 41.2 | 42.7 | | 61.4 | 30.3 |
| 62. | TA37666 | 50.3 | 47.6 | 48.2 | 49.7 | 48.4 | 47.6 | 44.5 | 46.1 | 48.8 | 51.0 | 48.2 | 49.5 | 70.7 | | 32.2 |
| 63. | TC253044 | 54.0 | 54.5 | 49.1 | 54.4 | 53.6 | 51.7 | 50.0 | 55.2 | 40.0 | 51.7 | 51.0 | 53.9 | 40.9 | 47.1 | |
| 64. | TabHLH11 | 63.3 | 63.2 | 59.5 | 65.9 | 66.1 | 65.6 | 83.3 | 60.5 | 43.0 | 46.2 | 65.2 | 64.5 | 41.6 | 45.5 | 51.4 |
| 65. | TA46156 | 85.7 | 87.0 | 65.0 | 66.9 | 67.8 | 82.5 | 65.1 | 60.2 | 42.5 | 48.9 | 82.5 | 69.4 | 41.6 | 47.6 | 53.8 |
| 66. | GSVIVT00017237001 | 45.2 | 44.7 | 45.0 | 43.1 | 44.7 | 44.7 | 41.4 | 45.7 | 55.9 | 47.4 | 44.5 | 45.2 | 67.1 | 64.4 | 43.5 |
| 67. | TA46194 | 73.8 | 73.5 | 74.8 | 72.5 | 73.2 | 74.4 | 66.8 | 56.9 | 44.3 | 50.5 | 74.4 | 73.5 | 45.2 | 50.5 | 51.1 |
| 68. | GSVIVT00016367001 | 36.3 | 37.9 | 37.9 | 36.7 | 37.3 | 38.8 | 34.6 | 37.3 | 44.5 | 40.6 | 38.6 | 37.1 | 46.8 | 42.7 | 41.4 |
| 69. | TA139285 | 61.7 | 61.2 | 64.1 | 66.2 | 65.5 | 64.2 | 82.5 | 59.6 | 42.8 | 48.6 | 64.6 | 65.8 | 43.2 | 44.0 | 54.8 |
| 70. | TA126400 | 65.0 | 64.9 | 62.0 | 66.2 | 64.5 | 61.9 | 92.3 | 59.3 | 42.5 | 48.0 | 61.9 | 65.5 | 40.7 | 46.1 | 53.8 |

| | | 64 | 65 | 66 | 67 | 68 | 69 | 70 |
|---|---|---|---|---|---|---|---|---|
| 1. | TC3698 | 56.1 | 53.5 | 34.9 | 54.6 | 25.8 | 58.1 | 55.7 |
| 2. | AT1G03040 | 50.6 | 63.2 | 33.5 | 53.4 | 28.0 | 50.8 | 49.4 |
| 3. | AT4G02590 | 53.7 | 69.0 | 34.8 | 56.3 | 28.5 | 53.7 | 50.7 |
| 4. | AT2G24260 | 17.0 | 16.3 | 22.1 | 16.5 | 16.3 | 16.8 | 16.5 |
| 5. | AT4G30980 | 39.5 | 38.6 | 39.7 | 37.3 | 29.5 | 40.2 | 40.5 |
| 6. | AT5G58010 | 37.3 | 36.1 | 36.2 | 35.9 | 28.6 | 38.4 | 37.5 |
| 7. | TC15501 | 51.1 | 59.6 | 34.8 | 64.3 | 30.7 | 53.4 | 49.7 |
| 8. | TC19278 | 19.9 | 21.3 | 20.0 | 20.1 | 19.5 | 20.2 | 20.1 |
| 9. | TC10015_part | 53.9 | 68.5 | 33.2 | 54.4 | 26.9 | 51.8 | 51.6 |
| 10. | DY268946 | 49.0 | 45.3 | 36.4 | 50.0 | 30.2 | 46.1 | 45.5 |
| 11. | TA2544 | 32.9 | 36.3 | 42.9 | 37.1 | 33.9 | 36.2 | 35.0 |
| 12. | TA3392 | 53.7 | 73.3 | 34.2 | 57.6 | 28.0 | 54.4 | 52.6 |
| 13. | TA12416 | 32.9 | 33.3 | 58.2 | 33.3 | 31.9 | 31.8 | 33.3 |
| 14. | WO051050seqID77 | 49.2 | 56.2 | 33.4 | 60.3 | 26.9 | 52.6 | 51.2 |
| 15. | WO051050seqID1671 | 48.9 | 66.8 | 34.0 | 57.0 | 29.8 | 49.6 | 48.9 |
| 16. | TA55042 | 49.0 | 51.4 | 34.2 | 56.0 | 28.4 | 52.0 | 51.2 |
| 17. | TC207545 | 54.5 | 67.5 | 33.2 | 55.4 | 26.1 | 54.6 | 55.6 |
| 18. | TA13791 | 54.7 | 75.6 | 36.5 | 59.8 | 29.1 | 54.6 | 53.7 |
| 19. | CO123623 | 53.0 | 49.4 | 30.9 | 55.6 | 27.3 | 52.5 | 51.9 |
| 20. | DT543504 | 37.9 | 36.0 | 31.4 | 34.5 | 37.8 | 34.8 | 35.2 |
| 21. | TC60118 | 54.4 | 75.2 | 36.5 | 59.5 | 29.1 | 54.2 | 53.4 |
| 22. | TC60119 | 53.9 | 75.7 | 35.6 | 59.4 | 29.0 | 53.9 | 51.2 |
| 23. | TC61833 | 53.1 | 73.4 | 35.2 | 59.2 | 28.6 | 53.8 | 52.5 |
| 24. | TC67603 | 32.6 | 34.9 | 51.4 | 34.0 | 31.0 | 32.4 | 33.9 |
| 25. | TC229602 | 51.4 | 63.3 | 29.4 | 53.2 | 23.3 | 51.4 | 50.0 |
| 26. | TC205173 | 38.1 | 39.7 | 52.3 | 40.4 | 30.7 | 37.6 | 38.8 |
| 27. | TA1140 | 52.1 | 54.3 | 35.5 | 61.0 | 27.7 | 54.6 | 52.8 |
| 28. | TC140470 | 25.1 | 24.9 | 29.7 | 28.1 | 34.4 | 25.2 | 25.5 |
| 29. | TA3490_part | 54.6 | 58.5 | 34.0 | 61.3 | 26.3 | 55.1 | 54.5 |
| 30. | CK293938 | 52.6 | 55.9 | 34.3 | 65.0 | 27.8 | 55.8 | 54.2 |
| 31. | TC8633 | 53.7 | 71.7 | 36.0 | 59.3 | 29.4 | 54.1 | 50.9 |
| 32. | TC7102 | 36.8 | 34.2 | 54.6 | 35.6 | 30.0 | 34.8 | 34.0 |
| 33. | TC7103 | 39.1 | 36.0 | 53.1 | 38.0 | 29.6 | 38.5 | 36.9 |
| 34. | TC12771 | 53.7 | 57.2 | 33.9 | 66.2 | 28.7 | 55.8 | 54.2 |
| 35. | Os03g58330 | 80.3 | 53.5 | 34.5 | 58.3 | 27.8 | 87.2 | 69.4 |
| 36. | Os06g08500 | 34.1 | 32.6 | 40.3 | 33.5 | 27.5 | 35.9 | 32.3 |
| 37. | Os06g09370 | 26.8 | 27.4 | 29.2 | 27.7 | 34.1 | 28.0 | 25.9 |
| 38. | Os02g55250 | 33.8 | 33.6 | 46.3 | 34.4 | 30.3 | 34.6 | 34.8 |
| 39. | Os02g35660 | 27.1 | 25.9 | 31.3 | 27.7 | 33.7 | 27.2 | 27.6 |
| 40. | Os07g08440 | 74.9 | 53.0 | 35.8 | 57.1 | 27.0 | 79.7 | 76.2 |
| 41. | Os09g25040 | 31.1 | 30.5 | 47.3 | 31.6 | 30.4 | 32.3 | 31.4 |
| 42. | Pt_scaff_II.416 | 53.9 | 74.1 | 34.4 | 61.5 | 28.9 | 55.3 | 52.2 |
| 43. | Pt_scaff_70.65 | 48.8 | 53.6 | 35.0 | 58.9 | 28.0 | 49.5 | 50.1 |
| 44. | Pt_scaff_XIII.403 | 47.5 | 50.3 | 35.8 | 57.4 | 29.5 | 47.6 | 48.1 |
| 45. | Pt_scaff28.86 | 28.1 | 28.3 | 34.3 | 31.0 | 49.3 | 29.8 | 26.4 |
| 46. | Pt_TC63334 | 54.8 | 76.6 | 35.1 | 60.0 | 28.5 | 55.6 | 53.1 |
| 47. | TA14134 | 32.0 | 32.4 | 33.7 | 32.5 | 29.4 | 32.9 | 32.2 |
| 48. | TA5414 | 32.9 | 33.8 | 57.9 | 33.8 | 32.1 | 32.6 | 32.8 |
| 49. | TA3263 | 55.1 | 76.9 | 35.6 | 60.0 | 28.3 | 55.6 | 53.4 |
| 50. | TA2825 | 55.8 | 77.7 | 35.8 | 62.0 | 28.0 | 55.5 | 53.7 |
| 51. | TA1616 | 51.4 | 56.6 | 34.4 | 62.5 | 29.2 | 55.0 | 54.4 |
| 52. | TA21665 | 52.1 | 52.9 | 33.2 | 60.5 | 27.6 | 53.6 | 52.7 |
| 53. | TC172581 | 51.9 | 57.3 | 35.3 | 64.2 | 28.6 | 53.1 | 51.6 |
| 54. | AK247217 | 54.0 | 73.9 | 35.3 | 60.2 | 30.1 | 53.3 | 50.9 |
| 55. | TC104646 | 74.4 | 54.6 | 34.2 | 58.1 | 26.3 | 77.6 | 91.2 |
| 56. | DV982110 | 45.8 | 45.4 | 37.3 | 43.8 | 29.7 | 46.7 | 45.9 |
| 57. | WO051050seqID516 | 33.4 | 35.4 | 40.4 | 36.1 | 29.6 | 34.8 | 33.3 |
| 58. | TC68930 | 32.2 | 32.1 | 35.4 | 32.3 | 30.7 | 34.3 | 34.6 |
| 59. | TA30646 | 54.7 | 73.9 | 35.5 | 60.2 | 29.5 | 54.9 | 51.2 |
| 60. | TA28621 | 56.2 | 35.3 | 63.5 | 28.7 | 53.4 | 52.2 | |
| 61. | TA34455 | 35.9 | 33.5 | 50.6 | 35.4 | 32.2 | 34.9 | 34.6 |
| 62. | TA37666 | 39.2 | 36.2 | 52.3 | 38.4 | 30.4 | 37.4 | 37.9 |
| 63. | TC253044 | 34.6 | 36.5 | 33.0 | 37.2 | 30.6 | 35.8 | 35.8 |
| 64. | TabHLH11 | 52.5 | 33.3 | 55.7 | 27.7 | 82.7 | 70.7 | |
| 65. | TA46156 | 66.4 | | 35.1 | 65.7 | 27.0 | 55.3 | 52.9 |
| 66. | GSVIVT00017237001 | 41.9 | 45.9 | | 36.0 | 31.6 | 32.5 | 35.6 |
| 67. | TA46194 | 66.1 | 75.7 | 45.0 | | 29.3 | 57.5 | 56.5 |
| 68. | GSVIVT00016367001 | 35.1 | 36.1 | 44.5 | 37.5 | | 26.9 | 26.1 |
| 69. | TA139285 | 89.5 | 66.4 | 40.0 | 68.1 | 37.3 | | 72.8 |
| 70. | TA126400 | 79.6 | 65.1 | 41.9 | 66.5 | 35.7 | 80.4 | |

TABLE E3
MatGAT results for global similarity and identity over the HLH domains of the bHLH11-like polypeptide sequences.

| | | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|
| 1. | AT2G24260 | | 98.0 | 92.2 | 98.0 | 98.0 | 98.0 | 98.0 | 98.0 | 98.0 | 94.1 | 94.1 | 96.1 | 86.3 | 98.0 | 88.9 | 96.1 | 98.0 |
| 2. | AT4G30980 | 100.0 | | 94.1 | 96.1 | 96.1 | 96.1 | 96.1 | 96.1 | 96.1 | 96.1 | 96.1 | 94.1 | 88.2 | 96.1 | 87.0 | 94.1 | 96.1 |
| 3. | AT5G58010 | 98.0 | 98.0 | | 92.2 | 92.2 | 92.2 | 92.2 | 92.2 | 92.2 | 96.1 | 96.1 | 90.2 | 84.3 | 92.2 | 83.3 | 90.2 | 92.2 |
| 4. | TA12416 | 98.0 | 98.0 | 98.0 | | 100.0 | 100.0 | 100.0 | 100.0 | 100.0 | 96.1 | 96.1 | 98.0 | 88.2 | 100.0 | 90.7 | 98.0 | 98.0 |
| 5. | TA5414 | 98.0 | 98.0 | 98.0 | 100.0 | | 100.0 | 100.0 | 100.0 | 100.0 | 96.1 | 96.1 | 98.0 | 88.2 | 100.0 | 90.7 | 98.0 | 98.0 |
| 6. | TC205173 | 98.0 | 98.0 | 98.0 | 100.0 | 100.0 | | 100.0 | 100.0 | 100.0 | 96.1 | 96.1 | 98.0 | 88.2 | 100.0 | 90.7 | 98.0 | 98.0 |
| 7. | GSVIVT00017237001 | 98.0 | 98.0 | 98.0 | 100.0 | 100.0 | 100.0 | | 100.0 | 100.0 | 96.1 | 96.1 | 98.0 | 88.2 | 100.0 | 90.7 | 98.0 | 98.0 |
| 8. | TC7102 | 98.0 | 98.0 | 98.0 | 100.0 | 100.0 | 100.0 | 100.0 | | 100.0 | 96.1 | 96.1 | 98.0 | 88.2 | 100.0 | 90.7 | 98.0 | 98.0 |
| 9. | TA34455 | 98.0 | 98.0 | 98.0 | 100.0 | 100.0 | 100.0 | 100.0 | 100.0 | | 96.1 | 96.1 | 98.0 | 88.2 | 100.0 | 90.7 | 98.0 | 98.0 |
| 10. | TC7103 | 98.0 | 98.0 | 98.0 | 100.0 | 100.0 | 100.0 | 100.0 | 100.0 | 100.0 | | 100.0 | 94.1 | 88.2 | 96.1 | 87.0 | 94.1 | 94.1 |
| 11. | TA37666 | 98.0 | 98.0 | 98.0 | 100.0 | 100.0 | 100.0 | 100.0 | 100.0 | 100.0 | 100.0 | | 94.1 | 88.2 | 96.1 | 87.0 | 94.1 | 94.1 |
| 12. | TC67603 | 96.1 | 96.1 | 96.1 | 98.0 | 98.0 | 98.0 | 98.0 | 98.0 | 98.0 | 98.0 | 98.0 | | 86.3 | 98.0 | 88.9 | 96.1 | 96.1 |
| 13. | Os06g08500 | 90.2 | 90.2 | 90.2 | 92.2 | 92.2 | 92.2 | 92.2 | 92.2 | 92.2 | 92.2 | 92.2 | 90.2 | | 88.2 | 79.6 | 86.3 | 86.3 |
| 14. | Os02g55250 | 98.0 | 98.0 | 98.0 | 100.0 | 100.0 | 100.0 | 100.0 | 100.0 | 100.0 | 100.0 | 100.0 | 98.0 | 92.2 | | 90.7 | 98.0 | 98.0 |
| 15. | Os09g25040 | 88.9 | 88.9 | 88.9 | 90.7 | 90.7 | 90.7 | 90.7 | 90.7 | 90.7 | 90.7 | 90.7 | 88.9 | 83.3 | 90.7 | | 88.9 | 88.9 |
| 16. | TA2544 | 98.0 | 98.0 | 98.0 | 100.0 | 100.0 | 100.0 | 100.0 | 100.0 | 100.0 | 100.0 | 100.0 | 98.0 | 92.2 | 100.0 | 90.7 | | 96.1 |
| 17. | DV982110 | 98.0 | 98.0 | 100.0 | 100.0 | 100.0 | 100.0 | 100.0 | 100.0 | 100.0 | 100.0 | 100.0 | 98.0 | 92.2 | 100.0 | 90.7 | 100.0 | |
| 18. | WO051050seqID516 | 98.0 | 98.0 | 100.0 | 100.0 | 100.0 | 100.0 | 100.0 | 100.0 | 100.0 | 100.0 | 100.0 | 98.0 | 92.2 | 100.0 | 90.7 | 100.0 | 100.0 |
| 19. | AT4G02590 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 20. | TC10015_part | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 21. | AT1G03040 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 22. | TA13791 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 23. | TC60118 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 24. | TC60119 | 94.1 | 94.1 | 94.1 | 94.1 | 94.1 | 94.1 | 94.1 | 94.1 | 94.1 | 94.1 | 94.1 | 92.2 | 86.3 | 94.1 | 85.2 | 94.1 | 94.1 |
| 25. | TC61833 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 26. | TA3392 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 27. | WO051050seqID1671 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 28. | Pt_TC63334 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 29. | TA3263 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 30. | Pt_scaff_II.416 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 31. | TA2825 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 32. | TC207545 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 33. | TC229602 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 34. | TA46156 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 35. | AK247217 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 36. | TA30646 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 37. | TC8633 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 38. | TC15501 | 98.0 | 98.0 | 100.0 | 100.0 | 100.0 | 100.0 | 100.0 | 100.0 | 100.0 | 100.0 | 100.0 | 98.0 | 92.2 | 100.0 | 90.7 | 100.0 | 100.0 |
| 39. | TA1140 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 40. | TA3490_part | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 41. | CK293938 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 42. | TC12771 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 43. | TC172581 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 44. | TA28621 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 45. | TA21665 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 46. | TA46194 | 98.0 | 98.0 | 100.0 | 100.0 | 100.0 | 100.0 | 100.0 | 100.0 | 100.0 | 100.0 | 100.0 | 98.0 | 92.2 | 100.0 | 90.7 | 100.0 | 100.0 |
| 47. | DY268946 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 48. | CO123623 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 49. | Pt_scaff_70.65 | 92.2 | 92.2 | 92.2 | 92.2 | 92.2 | 92.2 | 92.2 | 92.2 | 92.2 | 92.2 | 92.2 | 94.1 | 84.3 | 92.2 | 83.3 | 92.2 | 92.2 |
| 50. | Pt_scaff_XIII.403 | 92.2 | 92.2 | 92.2 | 92.2 | 92.2 | 92.2 | 92.2 | 92.2 | 92.2 | 92.2 | 92.2 | 94.1 | 84.3 | 92.2 | 83.3 | 92.2 | 92.2 |
| 51. | TA1616 | 96.1 | 96.1 | 96.1 | 98.0 | 98.0 | 98.0 | 98.0 | 98.0 | 98.0 | 98.0 | 98.0 | 96.1 | 90.2 | 98.0 | 88.9 | 98.0 | 98.0 |
| 52. | TA55042 | 98.0 | 98.0 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 53. | WO051050seqID77 | 96.1 | 96.1 | 96.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 54. | TC3698 | 96.1 | 96.1 | 98.0 | 96.1 | 96.1 | 96.1 | 96.1 | 96.1 | 96.1 | 96.1 | 96.1 | 94.1 | 88.2 | 96.1 | 87.0 | 96.1 | 98.0 |
| 55. | Os03g58330 | 98.0 | 98.0 | 100.0 | 98.0 | 98.0 | 98.0 | 98.0 | 98.0 | 98.0 | 98.0 | 98.0 | 96.1 | 90.2 | 98.0 | 88.9 | 98.0 | 100.0 |
| 56. | TA139285 | 98.0 | 98.0 | 100.0 | 98.0 | 98.0 | 98.0 | 98.0 | 98.0 | 98.0 | 98.0 | 98.0 | 96.1 | 90.2 | 98.0 | 88.9 | 98.0 | 100.0 |
| 57. | TabHLH11 | 96.1 | 96.1 | 98.0 | 96.1 | 96.1 | 96.1 | 96.1 | 96.1 | 96.1 | 96.1 | 96.1 | 94.1 | 88.2 | 96.1 | 87.0 | 96.1 | 98.0 |
| 58. | TC104646 | 98.0 | 98.0 | 100.0 | 98.0 | 98.0 | 98.0 | 98.0 | 98.0 | 98.0 | 98.0 | 98.0 | 96.1 | 90.2 | 98.0 | 88.9 | 98.0 | 100.0 |
| 59. | TA126400 | 98.0 | 98.0 | 100.0 | 98.0 | 98.0 | 98.0 | 98.0 | 98.0 | 98.0 | 98.0 | 98.0 | 96.1 | 90.2 | 98.0 | 88.9 | 98.0 | 100.0 |
| 60. | Os07g08440 | 98.0 | 98.0 | 100.0 | 98.0 | 98.0 | 98.0 | 98.0 | 98.0 | 98.0 | 98.0 | 98.0 | 96.1 | 90.2 | 98.0 | 88.9 | 98.0 | 100.0 |
| 61. | Os02g35660 | 96.1 | 96.1 | 98.0 | 98.0 | 98.0 | 98.0 | 98.0 | 98.0 | 98.0 | 98.0 | 98.0 | 96.1 | 90.2 | 98.0 | 88.9 | 98.0 | 98.0 |
| 62. | DT543504 | 94.1 | 96.1 | 98.0 | 96.1 | 96.1 | 96.1 | 96.1 | 96.1 | 96.1 | 98.0 | 98.0 | 94.1 | 90.2 | 96.1 | 87.0 | 96.1 | 96.1 |
| 63. | Pt_scaff28.86 | 94.1 | 96.1 | 98.0 | 96.1 | 96.1 | 96.1 | 96.1 | 96.1 | 96.1 | 98.0 | 98.0 | 94.1 | 90.2 | 96.1 | 88.9 | 96.1 | 96.1 |
| 64. | GSVIVT00016367001 | 96.1 | 98.0 | 100.0 | 98.0 | 98.0 | 98.0 | 98.0 | 98.0 | 98.0 | 100.0 | 100.0 | 96.1 | 92.2 | 98.0 | 88.9 | 98.0 | 98.0 |
| 65. | TC19278 | 68.6 | 70.6 | 72.5 | 70.6 | 70.6 | 70.6 | 70.6 | 70.6 | 70.6 | 72.5 | 72.5 | 70.6 | 72.5 | 70.6 | 63.0 | 70.6 | 70.6 |
| 66. | TA14134 | 94.1 | 94.1 | 94.1 | 96.1 | 96.1 | 96.1 | 96.1 | 96.1 | 96.1 | 96.1 | 96.1 | 94.1 | 88.2 | 96.1 | 87.0 | 96.1 | 96.1 |
| 67. | TC68930 | 98.0 | 98.0 | 100.0 | 100.0 | 100.0 | 100.0 | 100.0 | 100.0 | 100.0 | 100.0 | 100.0 | 98.0 | 92.2 | 100.0 | 90.7 | 100.0 | 100.0 |
| 68. | TC140470 | 94.1 | 96.1 | 98.0 | 96.1 | 96.1 | 96.1 | 96.1 | 96.1 | 96.1 | 98.0 | 98.0 | 94.1 | 90.2 | 96.1 | 87.0 | 96.1 | 96.1 |
| 69. | TC253044 | 94.1 | 96.1 | 98.0 | 96.1 | 96.1 | 96.1 | 96.1 | 96.1 | 96.1 | 98.0 | 98.0 | 94.1 | 90.2 | 96.1 | 87.0 | 96.1 | 96.1 |
| 70. | Os06g09370 | 92.2 | 94.1 | 96.1 | 94.1 | 94.1 | 94.1 | 94.1 | 94.1 | 94.1 | 96.1 | 96.1 | 92.2 | 88.2 | 94.1 | 85.2 | 94.1 | 94.1 |
| | | 18 | 19 | 20 | 21 | 22 | 23 | 24 | 25 | 26 | 27 | 28 | 29 | 30 | 31 | 32 | 33 | 34 |
| 1. | AT2G24260 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 2. | AT4G30980 | 96.1 | 80.4 | 80.4 | 82.4 | 82.4 | 82.4 | 80.4 | 82.4 | 82.4 | 80.4 | 80.4 | 82.4 | 80.4 | 82.4 | 82.4 | 82.4 | 82.4 |
| 3. | AT5G58010 | 92.2 | 76.5 | 76.5 | 78.4 | 78.4 | 78.4 | 76.5 | 78.4 | 78.4 | 76.5 | 76.5 | 78.4 | 76.5 | 78.4 | 78.4 | 78.4 | 78.4 |
| 4. | TA12416 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 5. | TA5414 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 6. | TC205173 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 7. | GSVIVT00017237001 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 8. | TC7102 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 9. | TA34455 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 10. | TC7103 | 94.1 | 78.4 | 78.4 | 80.4 | 80.4 | 80.4 | 78.4 | 80.4 | 80.4 | 78.4 | 78.4 | 80.4 | 78.4 | 80.4 | 80.4 | 80.4 | 80.4 |
| 11. | TA37666 | 94.1 | 78.4 | 78.4 | 80.4 | 80.4 | 80.4 | 78.4 | 80.4 | 80.4 | 78.4 | 78.4 | 80.4 | 78.4 | 80.4 | 80.4 | 80.4 | 80.4 |
| 12. | TC67603 | 96.1 | 80.4 | 80.4 | 78.4 | 82.4 | 82.4 | 80.4 | 82.4 | 82.4 | 78.4 | 80.4 | 82.4 | 80.4 | 82.4 | 82.4 | 82.4 | 82.4 |
| 13. | Os06g08500 | 86.3 | 70.6 | 70.6 | 72.5 | 72.5 | 72.5 | 70.6 | 72.5 | 72.5 | 70.6 | 70.6 | 72.5 | 70.6 | 72.5 | 72.5 | 72.5 | 72.5 |
| 14. | Os02g55250 | 98.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 15. | Os09g25040 | 88.9 | 74.1 | 74.1 | 72.2 | 75.9 | 75.9 | 74.1 | 75.9 | 75.9 | 72.2 | 74.1 | 75.9 | 75.9 | 75.9 | 75.9 | 75.9 | 75.9 |
| 16. | TA2544 | 96.1 | 80.4 | 80.4 | 78.4 | 82.4 | 82.4 | 80.4 | 82.4 | 82.4 | 78.4 | 80.4 | 82.4 | 80.4 | 82.4 | 82.4 | 82.4 | 82.4 |
| 17. | DV982110 | 100.0 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 |
| 18. | WO051050seqID516 | 82.4 | 82.4 | 80.4 | 84.3 | 84.3 | 82.4 | 84.3 | 84.3 | 80.4 | 82.4 | 84.3 | 82.4 | 84.3 | 84.3 | 84.3 | 84.3 | |
| 19. | AT4G02590 | 96.1 | | 100.0 | 98.0 | 96.1 | 96.1 | 92.2 | 94.1 | 96.1 | 92.2 | 92.2 | 94.1 | 94.1 | 96.1 | 96.1 | 96.1 | 96.1 |
| 20. | TC10015_part | 96.1 | 100.0 | | 98.0 | 96.1 | 96.1 | 92.2 | 94.1 | 96.1 | 92.2 | 92.2 | 94.1 | 94.1 | 96.1 | 96.1 | 96.1 | 96.1 |
| 21. | AT1G03040 | 96.1 | 100.0 | 100.0 | | 94.1 | 94.1 | 90.2 | 92.2 | 94.1 | 92.2 | 90.2 | 92.2 | 92.2 | 94.1 | 94.1 | 94.1 | 94.1 |
| 22. | TA13791 | 98.0 | 100.0 | 100.0 | 100.0 | | 100.0 | 96.1 | 98.0 | 100.0 | 96.1 | 96.1 | 98.0 | 98.0 | 100.0 | 100.0 | 100.0 | 100.0 |
| 23. | TC60118 | 98.0 | 100.0 | 100.0 | 100.0 | 100.0 | | 96.1 | 98.0 | 100.0 | 96.1 | 96.1 | 98.0 | 98.0 | 100.0 | 100.0 | 100.0 | 100.0 |
| 24. | TC60119 | 94.1 | 96.1 | 96.1 | 96.1 | 96.1 | 96.1 | | 98.0 | 96.1 | 92.2 | 94.1 | 96.1 | 94.1 | 96.1 | 96.1 | 96.1 | 96.1 |
| 25. | TC61833 | 96.1 | 98.0 | 98.0 | 98.0 | 98.0 | 98.0 | 98.0 | | 98.0 | 94.1 | 96.1 | 98.0 | 96.1 | 98.0 | 98.0 | 98.0 | 98.0 |
| 26. | TA3392 | 98.0 | 100.0 | 100.0 | 100.0 | 100.0 | 100.0 | 96.1 | 98.0 | | 96.1 | 96.1 | 98.0 | 98.0 | 100.0 | 100.0 | 100.0 | 100.0 |
| 27. | WO051050seqID1671 | 96.1 | 98.0 | 98.0 | 98.0 | 98.0 | 98.0 | 94.1 | 96.1 | 98.0 | | 92.2 | 94.1 | 94.1 | 96.1 | 96.1 | 96.1 | 96.1 |
| 28. | Pt_TC63334 | 96.1 | 96.1 | 96.1 | 96.1 | 96.1 | 96.1 | 94.1 | 96.1 | 96.1 | 94.1 | | 98.0 | 94.1 | 96.1 | 96.1 | 96.1 | 96.1 |
| 29. | TA3263 | 98.0 | 98.0 | 98.0 | 98.0 | 98.0 | 98.0 | 96.1 | 98.0 | 98.0 | 96.1 | 98.0 | | 96.1 | 98.0 | 98.0 | 98.0 | 98.0 |
| 30. | Pt_scaff_II.416 | 98.0 | 100.0 | 100.0 | 100.0 | 100.0 | 100.0 | 96.1 | 98.0 | 100.0 | 98.0 | 96.1 | 98.0 | | 98.0 | 98.0 | 98.0 | 98.0 |
| 31. | TA2825 | 98.0 | 100.0 | 100.0 | 100.0 | 100.0 | 100.0 | 96.1 | 98.0 | 100.0 | 98.0 | 96.1 | 98.0 | 100.0 | | 100.0 | 100.0 | 100.0 |
| 32. | TC207545 | 98.0 | 100.0 | 100.0 | 100.0 | 100.0 | 100.0 | 96.1 | 98.0 | 100.0 | 98.0 | 96.1 | 98.0 | 100.0 | 100.0 | | 100.0 | 100.0 |
| 33. | TC229602 | 98.0 | 100.0 | 100.0 | 100.0 | 100.0 | 100.0 | 96.1 | 98.0 | 100.0 | 98.0 | 96.1 | 98.0 | 100.0 | 100.0 | 100.0 | | 100.0 |
| 34. | TA46156 | 98.0 | 100.0 | 100.0 | 100.0 | 100.0 | 100.0 | 96.1 | 98.0 | 100.0 | 98.0 | 96.1 | 98.0 | 100.0 | 100.0 | 100.0 | 100.0 | |
| 35. | AK247217 | 96.1 | 98.0 | 98.0 | 98.0 | 98.0 | 98.0 | 98.0 | 100.0 | 98.0 | 96.1 | 96.1 | 98.0 | 98.0 | 98.0 | 98.0 | 98.0 | 98.0 |
| 36. | TA30646 | 96.1 | 98.0 | 98.0 | 98.0 | 98.0 | 98.0 | 98.0 | 100.0 | 98.0 | 96.1 | 96.1 | 98.0 | 98.0 | 98.0 | 98.0 | 98.0 | 98.0 |
| 37. | TC8633 | 96.1 | 96.1 | 96.1 | 96.1 | 98.0 | 98.0 | 98.0 | 100.0 | 98.0 | 96.1 | 96.1 | 98.0 | 98.0 | 98.0 | 98.0 | 98.0 | 98.0 |
| 38. | TC15501 | 100.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 39. | TA1140 | 98.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 40. | TA3490_part | 98.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 41. | CK293938 | 98.0 | 98.0 | 98.0 | 98.0 | 98.0 | 98.0 | 96.1 | 96.1 | 98.0 | 96.1 | 94.1 | 96.1 | 98.0 | 98.0 | 98.0 | 98.0 | 98.0 |
| 42. | TC12771 | 98.0 | 98.0 | 98.0 | 98.0 | 98.0 | 98.0 | 96.1 | 96.1 | 98.0 | 96.1 | 94.1 | 96.1 | 98.0 | 98.0 | 98.0 | 98.0 | 98.0 |
| 43. | TC172581 | 98.0 | 98.0 | 98.0 | 98.0 | 98.0 | 98.0 | 96.1 | 96.1 | 98.0 | 96.1 | 94.1 | 96.1 | 98.0 | 98.0 | 98.0 | 98.0 | 98.0 |
| 44. | TA28621 | 98.0 | 98.0 | 98.0 | 98.0 | 98.0 | 98.0 | 96.1 | 96.1 | 98.0 | 96.1 | 94.1 | 96.1 | 98.0 | 98.0 | 98.0 | 98.0 | 98.0 |
| 45. | TA21665 | 98.0 | 98.0 | 98.0 | 98.0 | 98.0 | 98.0 | 96.1 | 96.1 | 98.0 | 96.1 | 94.1 | 96.1 | 98.0 | 98.0 | 98.0 | 98.0 | 98.0 |
| 46. | TA46194 | 100.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 47. | DY268946 | 98.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 48. | CO123623 | 98.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 49. | Pt_scaff_70.65 | 92.2 | 94.1 | 94.1 | 94.1 | 94.1 | 94.1 | 94.1 | 92.2 | 94.1 | 92.2 | 90.2 | 92.2 | 94.1 | 94.1 | 94.1 | 94.1 | 94.1 |
| 50. | Pt_scaff_XIII.403 | 92.2 | 94.1 | 94.1 | 94.1 | 94.1 | 94.1 | 94.1 | 92.2 | 94.1 | 92.2 | 90.2 | 92.2 | 94.1 | 94.1 | 94.1 | 94.1 | 94.1 |
| 51. | TA1616 | 98.0 | 98.0 | 98.0 | 98.0 | 98.0 | 98.0 | 94.1 | 96.1 | 98.0 | 96.1 | 94.1 | 96.1 | 98.0 | 98.0 | 98.0 | 98.0 | 98.0 |
| 52. | TA55042 | 98.0 | 100.0 | 100.0 | 100.0 | 100.0 | 100.0 | 96.1 | 98.0 | 100.0 | 98.0 | 96.1 | 98.0 | 100.0 | 100.0 | 100.0 | 100.0 | 100.0 |
| 53. | WO051050seqID77 | 96.1 | 96.1 | 96.1 | 96.1 | 96.1 | 96.1 | 94.1 | 94.1 | 96.1 | 94.1 | 92.2 | 94.1 | 96.1 | 96.1 | 96.1 | 96.1 | 96.1 |
| 54. | TC3698 | 98.0 | 96.1 | 96.1 | 96.1 | 96.1 | 96.1 | 96.1 | 98.0 | 96.1 | 94.1 | 94.1 | 96.1 | 96.1 | 96.1 | 96.1 | 96.1 | 96.1 |
| 55. | Os03g58330 | 100.0 | 96.1 | 96.1 | 96.1 | 98.0 | 98.0 | 94.1 | 96.1 | 98.0 | 96.1 | 94.1 | 96.1 | 98.0 | 98.0 | 98.0 | 98.0 | 98.0 |
| 56. | TA139285 | 100.0 | 96.1 | 96.1 | 96.1 | 98.0 | 98.0 | 94.1 | 96.1 | 98.0 | 96.1 | 94.1 | 96.1 | 98.0 | 98.0 | 98.0 | 98.0 | 98.0 |
| 57. | TabHLH11 | 98.0 | 94.1 | 94.1 | 94.1 | 96.1 | 96.1 | 96.1 | 98.0 | 96.1 | 94.1 | 94.1 | 96.1 | 96.1 | 96.1 | 96.1 | 96.1 | 96.1 |
| 58. | TC104646 | 100.0 | 96.1 | 96.1 | 96.1 | 98.0 | 98.0 | 94.1 | 96.1 | 98.0 | 96.1 | 94.1 | 96.1 | 98.0 | 98.0 | 98.0 | 98.0 | 98.0 |
| 59. | TA126400 | 100.0 | 96.1 | 96.1 | 96.1 | 98.0 | 98.0 | 94.1 | 96.1 | 98.0 | 96.1 | 94.1 | 96.1 | 98.0 | 98.0 | 98.0 | 98.0 | 98.0 |
| 60. | Os07g08440 | 100.0 | 96.1 | 96.1 | 96.1 | 98.0 | 98.0 | 94.1 | 96.1 | 98.0 | 96.1 | 94.1 | 96.1 | 98.0 | 98.0 | 98.0 | 98.0 | 98.0 |
| 61. | Os02g35660 | 98.0 | 94.1 | 94.1 | 94.1 | 96.1 | 96.1 | 94.1 | 94.1 | 96.1 | 96.1 | 94.1 | 96.1 | 96.1 | 96.1 | 96.1 | 96.1 | 96.1 |
| 62. | DT543504 | 96.1 | 92.2 | 92.2 | 94.1 | 94.1 | 94.1 | 90.2 | 92.2 | 94.1 | 94.1 | 92.2 | 94.1 | 94.1 | 94.1 | 94.1 | 94.1 | 94.1 |
| 63. | Pt_scaff28.86 | 96.1 | 92.2 | 92.2 | 94.1 | 94.1 | 94.1 | 90.2 | 92.2 | 94.1 | 94.1 | 92.2 | 94.1 | 94.1 | 94.1 | 94.1 | 94.1 | 94.1 |
| 64. | GSVIVT00016367001 | 98.0 | 94.1 | 94.1 | 96.1 | 96.1 | 96.1 | 92.2 | 94.1 | 96.1 | 96.1 | 94.1 | 96.1 | 96.1 | 96.1 | 96.1 | 96.1 | 96.1 |
| 65. | TC19278 | 70.6 | 66.7 | 66.7 | 68.6 | 68.6 | 68.6 | 66.7 | 66.7 | 68.6 | 68.6 | 66.7 | 68.6 | 68.6 | 68.6 | 68.6 | 68.6 | 68.6 |
| 66. | TA14134 | 96.1 | 92.2 | 92.2 | 92.2 | 94.1 | 94.1 | 90.2 | 92.2 | 94.1 | 92.2 | 92.2 | 94.1 | 94.1 | 94.1 | 94.1 | 94.1 | 94.1 |
| 67. | TC68930 | 100.0 | 96.1 | 96.1 | 96.1 | 98.0 | 98.0 | 94.1 | 96.1 | 98.0 | 96.1 | 96.1 | 98.0 | 98.0 | 98.0 | 98.0 | 98.0 | 98.0 |
| 68. | TC140470 | 96.1 | 92.2 | 92.2 | 94.1 | 94.1 | 94.1 | 92.2 | 92.2 | 94.1 | 94.1 | 92.2 | 94.1 | 94.1 | 94.1 | 94.1 | 94.1 | 94.1 |
| 69. | TC253044 | 96.1 | 92.2 | 92.2 | 94.1 | 94.1 | 94.1 | 92.2 | 92.2 | 94.1 | 94.1 | 92.2 | 94.1 | 94.1 | 94.1 | 94.1 | 94.1 | 94.1 |
| 70. | Os06g09370 | 94.1 | 90.2 | 90.2 | 92.2 | 92.2 | 92.2 | 90.2 | 90.2 | 92.2 | 92.2 | 90.2 | 92.2 | 92.2 | 92.2 | 92.2 | 92.2 | 92.2 |
| | | 35 | 36 | 37 | 38 | 39 | 40 | 41 | 42 | 43 | 44 | 45 | 46 | 47 | 48 | 49 | 50 | 51 |
| 1. | AT2G24260 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 86.3 |
| 2. | AT4G30980 | 80.4 | 82.4 | 84.3 | 86.3 | 86.3 | 86.3 | 82.4 | 82.4 | 82.4 | 82.4 | 82.4 | 86.3 | 82.4 | 86.3 | 80.4 | 78.4 | 84.3 |
| 3. | AT5G58010 | 76.5 | 78.4 | 80.4 | 82.4 | 82.4 | 82.4 | 78.4 | 78.4 | 78.4 | 78.4 | 78.4 | 82.4 | 78.4 | 82.4 | 76.5 | 74.5 | 80.4 |
| 4. | TA12416 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 5. | TA5414 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 6. | TC205173 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 7. | GSVIVT00017237001 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 8. | TC7102 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 9. | TA34455 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 10. | TC7103 | 78.4 | 80.4 | 82.4 | 84.3 | 84.3 | 84.3 | 80.4 | 80.4 | 80.4 | 80.4 | 80.4 | 84.3 | 80.4 | 84.3 | 78.4 | 76.5 | 84.3 |
| 11. | TA37666 | 78.4 | 80.4 | 82.4 | 84.3 | 84.3 | 84.3 | 80.4 | 80.4 | 80.4 | 80.4 | 80.4 | 84.3 | 80.4 | 84.3 | 78.4 | 76.5 | 84.3 |
| 12. | TC67603 | 80.4 | 82.4 | 84.3 | 86.3 | 86.3 | 86.3 | 82.4 | 82.4 | 82.4 | 82.4 | 82.4 | 86.3 | 82.4 | 86.3 | 84.3 | 82.4 | 86.3 |
| 13. | Os06g08500 | 70.6 | 72.5 | 74.5 | 76.5 | 76.5 | 76.5 | 72.5 | 72.5 | 72.5 | 72.5 | 72.5 | 76.5 | 72.5 | 76.5 | 70.6 | 68.6 | 76.5 |
| 14. | Os02g55250 | 82.4 | 84.3 | 86.3 | 88.2 | 88.2 | 88.2 | 84.3 | 84.3 | 84.3 | 84.3 | 84.3 | 88.2 | 84.3 | 88.2 | 82.4 | 80.4 | 88.2 |
| 15. | Os09g25040 | 74.1 | 75.9 | 77.8 | 79.6 | 79.6 | 79.6 | 75.9 | 75.9 | 75.9 | 75.9 | 75.9 | 79.6 | 75.9 | 79.6 | 74.1 | 72.2 | 79.6 |
| 16. | TA2544 | 82.4 | 82.4 | 84.3 | 86.3 | 86.3 | 86.3 | 82.4 | 82.4 | 82.4 | 82.4 | 82.4 | 86.3 | 82.4 | 86.3 | 80.4 | 78.4 | 86.3 |
| 17. | DV982110 | 82.4 | 84.3 | 86.3 | 90.2 | 88.2 | 88.2 | 84.3 | 84.3 | 84.3 | 84.3 | 84.3 | 90.2 | 84.3 | 88.2 | 82.4 | 80.4 | 86.3 |
| 18. | WO051050seqID516 | 82.4 | 84.3 | 86.3 | 90.2 | 88.2 | 88.2 | 84.3 | 84.3 | 84.3 | 84.3 | 84.3 | 90.2 | 84.3 | 88.2 | 82.4 | 80.4 | 86.3 |
| 19. | AT4G02590 | 92.2 | 94.1 | 94.1 | 90.2 | 88.2 | 88.2 | 88.2 | 88.2 | 88.2 | 88.2 | 88.2 | 90.2 | 90.2 | 90.2 | 88.2 | 86.3 | 94.1 |
| 20. | TC10015_part | 92.2 | 94.1 | 94.1 | 90.2 | 88.2 | 88.2 | 88.2 | 88.2 | 88.2 | 88.2 | 88.2 | 90.2 | 90.2 | 90.2 | 88.2 | 86.3 | 94.1 |
| 21. | AT1G03040 | 90.2 | 92.2 | 92.2 | 88.2 | 86.3 | 86.3 | 86.3 | 86.3 | 86.3 | 86.3 | 86.3 | 88.2 | 88.2 | 88.2 | 86.3 | 84.3 | 92.2 |
| 22. | TA13791 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 23. | TC60118 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 24. | TC60119 | 96.1 | 98.0 | 94.1 | 90.2 | 88.2 | 88.2 | 90.2 | 90.2 | 90.2 | 90.2 | 90.2 | 90.2 | 90.2 | 90.2 | 88.2 | 86.3 | 86.3 |
| 25. | TC61833 | 98.0 | 100.0 | 96.1 | 92.2 | 90.2 | 90.2 | 90.2 | 90.2 | 90.2 | 90.2 | 90.2 | 92.2 | 92.2 | 92.2 | 86.3 | 84.3 | 88.2 |
| 26. | TA3392 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 27. | WO051050seqID1671 | 92.2 | 94.1 | 92.2 | 90.2 | 88.2 | 88.2 | 88.2 | 88.2 | 88.2 | 88.2 | 88.2 | 90.2 | 90.2 | 90.2 | 84.3 | 82.4 | 86.3 |
| 28. | Pt_TC63334 | 94.1 | 96.1 | 94.1 | 90.2 | 88.2 | 88.2 | 88.2 | 88.2 | 88.2 | 88.2 | 88.2 | 90.2 | 90.2 | 90.2 | 84.3 | 82.4 | 86.3 |
| 29. | TA3263 | 96.1 | 98.0 | 96.1 | 92.2 | 90.2 | 90.2 | 90.2 | 90.2 | 90.2 | 90.2 | 90.2 | 92.2 | 92.2 | 92.2 | 86.3 | 84.3 | 88.2 |
| 30. | Pt_scaff_II.416 | 94.1 | 96.1 | 94.1 | 92.2 | 90.2 | 90.2 | 90.2 | 90.2 | 90.2 | 90.2 | 90.2 | 92.2 | 92.2 | 92.2 | 86.3 | 84.3 | 88.2 |
| 31. | TA2825 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 32. | TC207545 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 33. | TC229602 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 34. | TA46156 | 96.1 | 98.0 | 96.1 | 94.1 | 92.2 | 92.2 | 92.2 | 92.2 | 92.2 | 92.2 | 92.2 | 94.1 | 94.1 | 94.1 | 88.2 | 86.3 | 90.2 |
| 35. | AK247217 | | 98.0 | 94.1 | 90.2 | 88.2 | 88.2 | 88.2 | 88.2 | 88.2 | 88.2 | 88.2 | 90.2 | 90.2 | 90.2 | 84.3 | 82.4 | 86.3 |
| 36. | TA30646 | 100.0 | 96.1 | 92.2 | 90.2 | 90.2 | 90.2 | 90.2 | 90.2 | 90.2 | 90.2 | 92.2 | 92.2 | 92.2 | 86.3 | 84.3 | 88.2 | |
| 37. | TC8633 | 100.0 | 100.0 | | 90.2 | 88.2 | 88.2 | 88.2 | 88.2 | 88.2 | 88.2 | 88.2 | 90.2 | 90.2 | 90.2 | 86.3 | 84.3 | 88.2 |
| 36. | TC15501 | 96.1 | 96.1 | 96.1 | | 92.2 | 92.2 | 92.2 | 92.2 | 92.2 | 92.2 | 92.2 | 100.0 | 94.1 | 98.0 | 90.2 | 88.2 | 94.1 |
| 39. | TA1140 | 96.1 | 96.1 | 96.1 | 98.0 | | 100.0 | 92.2 | 92.2 | 92.2 | 92.2 | 92.2 | 92.2 | 94.1 | 94.1 | 86.3 | 84.3 | 88.2 |
| 40. | TA3490_part | 96.1 | 96.1 | 96.1 | 98.0 | 100.0 | | 92.2 | 92.2 | 92.2 | 92.2 | 92.2 | 92.2 | 94.1 | 94.1 | 86.3 | 84.3 | 88.2 |
| 41. | CK293938 | 96.1 | 96.1 | 96.1 | 98.0 | 100.0 | 100.0 | | 100.0 | 100.0 | 100.0 | 100.0 | 92.2 | 90.2 | 94.1 | 90.2 | 88.2 | 88.2 |
| 42. | TC12771 | 96.1 | 96.1 | 96.1 | 98.0 | 100.0 | 100.0 | 100.0 | | 100.0 | 100.0 | 100.0 | 92.2 | 90.2 | 94.1 | 90.2 | 88.2 | 88.2 |
| 43. | TC172581 | 96.1 | 96.1 | 96.1 | 98.0 | 100.0 | 100.0 | 100.0 | 100.0 | | 100.0 | 100.0 | 92.2 | 90.2 | 94.1 | 90.2 | 88.2 | 88.2 |
| 44. | TA28621 | 96.1 | 96.1 | 96.1 | 98.0 | 100.0 | 100.0 | 100.0 | 100.0 | 100.0 | | 100.0 | 92.2 | 90.2 | 94.1 | 90.2 | 88.2 | 88.2 |
| 45. | TA21665 | 96.1 | 96.1 | 96.1 | 98.0 | 100.0 | 100.0 | 100.0 | 100.0 | 100.0 | 100.0 | | 92.2 | 90.2 | 94.1 | 90.2 | 88.2 | 88.2 |
| 46. | TA46194 | 96.1 | 96.1 | 96.1 | 100.0 | 98.0 | 98.0 | 98.0 | 98.0 | 98.0 | 98.0 | 98.0 | | 94.1 | 98.0 | 90.2 | 88.2 | 94.1 |
| 47. | DY268946 | 96.1 | 96.1 | 96.1 | 98.0 | 100.0 | 100.0 | 100.0 | 100.0 | 100.0 | 100.0 | 100.0 | 98.0 | | 96.1 | 88.2 | 86.3 | 90.2 |
| 48. | CO123623 | 96.1 | 96.1 | 96.1 | 98.0 | 100.0 | 100.0 | 100.0 | 100.0 | 100.0 | 100.0 | 100.0 | 98.0 | 100.0 | | 92.2 | 90.2 | 94.1 |
| 49. | Pt_scaff_70.65 | 92.2 | 92.2 | 90.2 | 94.1 | 96.1 | 96.1 | 98.0 | 98.0 | 98.0 | 98.0 | 98.0 | 94.1 | 96.1 | 96.1 | | 98.0 | 90.2 |
| 50. | Pt_scaff_XIII.403 | 92.2 | 92.2 | 90.2 | 94.1 | 96.1 | 96.1 | 98.0 | 98.0 | 98.0 | 98.0 | 98.0 | 94.1 | 96.1 | 96.1 | 100.0 | | 88.2 |
| 51. | TA1616 | 96.1 | 96.1 | 94.1 | 100.0 | 98.0 | 98.0 | 98.0 | 98.0 | 98.0 | 98.0 | 98.0 | 100.0 | 98.0 | 98.0 | 94.1 | 94.1 | |
| 52. | TA55042 | 98.0 | 98.0 | 98.0 | 98.0 | 98.0 | 98.0 | 98.0 | 98.0 | 98.0 | 98.0 | 98.0 | 98.0 | 98.0 | 98.0 | 94.1 | 94.1 | 98.0 |
| 53. | WO051050seqID77 | 94.1 | 94.1 | 94.1 | 96.1 | 98.0 | 98.0 | 100.0 | 100.0 | 100.0 | 100.0 | 100.0 | 96.1 | 98.0 | 98.0 | 96.1 | 96.1 | 96.1 |
| 54. | TC3698 | 98.0 | 98.0 | 98.0 | 98.0 | 96.1 | 96.1 | 96.1 | 96.1 | 96.1 | 96.1 | 96.1 | 98.0 | 96.1 | 96.1 | 92.2 | 92.2 | 96.1 |
| 55. | Os03g58330 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 96.1 |
| 56. | TA139285 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 96.1 |
| 57. | TabHLH11 | 98.0 | 98.0 | 98.0 | 98.0 | 96.1 | 96.1 | 96.1 | 96.1 | 96.1 | 96.1 | 96.1 | 98.0 | 96.1 | 96.1 | 90.2 | 90.2 | 94.1 |
| 58. | TC104646 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 96.1 |
| 59. | TA126400 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 96.1 |
| 60. | Os07g08440 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 96.1 |
| 61. | Os02g35660 | 94.1 | 94.1 | 94.1 | 98.0 | 96.1 | 96.1 | 96.1 | 96.1 | 96.1 | 96.1 | 96.1 | 98.0 | 96.1 | 96.1 | 92.2 | 92.2 | 96.1 |
| 62. | DT543504 | 92.2 | 92.2 | 92.2 | 96.1 | 94.1 | 94.1 | 94.1 | 94.1 | 94.1 | 94.1 | 94.1 | 96.1 | 94.1 | 94.1 | 88.2 | 88.2 | 94.1 |
| 63. | Pt_scaff28.86 | 92.2 | 92.2 | 92.2 | 96.1 | 94.1 | 94.1 | 94.1 | 94.1 | 94.1 | 94.1 | 94.1 | 96.1 | 94.1 | 94.1 | 88.2 | 88.2 | 94.1 |
| 64. | GSVIVT00016367001 | 94.1 | 94.1 | 94.1 | 98.0 | 96.1 | 96.1 | 96.1 | 96.1 | 96.1 | 96.1 | 96.1 | 98.0 | 96.1 | 96.1 | 90.2 | 90.2 | 96.1 |
| 65. | TC19278 | 66.7 | 66.7 | 66.7 | 70.6 | 68.6 | 68.6 | 68.6 | 68.6 | 68.6 | 68.6 | 68.6 | 70.6 | 68.6 | 68.6 | 66.7 | 66.7 | 68.6 |
| 66. | TA14134 | 92.2 | 92.2 | 92.2 | 96.1 | 94.1 | 94.1 | 94.1 | 94.1 | 94.1 | 94.1 | 94.1 | 96.1 | 92.2 | 94.1 | 88.2 | 88.2 | 94.1 |
| 67. | TC68930 | 96.1 | 96.1 | 96.1 | 100.0 | 98.0 | 98.0 | 98.0 | 98.0 | 98.0 | 98.0 | 98.0 | 100.0 | 98.0 | 98.0 | 92.2 | 92.2 | 98.0 |
| 68. | TC140470 | 92.2 | 92.2 | 92.2 | 96.1 | 94.1 | 94.1 | 94.1 | 94.1 | 94.1 | 94.1 | 94.1 | 96.1 | 94.1 | 94.1 | 90.2 | 90.2 | 94.1 |
| 69. | TC253044 | 92.2 | 92.2 | 92.2 | 96.1 | 94.1 | 94.1 | 94.1 | 94.1 | 94.1 | 94.1 | 94.1 | 96.1 | 94.1 | 94.1 | 90.2 | 90.2 | 94.1 |
| 70. | Os06g09370 | 90.2 | 90.2 | 90.2 | 94.1 | 92.2 | 92.2 | 92.2 | 92.2 | 92.2 | 92.2 | 92.2 | 94.1 | 92.2 | 92.2 | 88.2 | 88.2 | 92.2 |

| | | 52 | 53 | 54 | 55 | 56 | 57 | 58 | 59 | 60 | 61 | 62 | 63 |
|---|---|---|---|---|---|---|---|---|---|---|---|---|---|
| 1. | AT2G24260 | 88.2 | 86.3 | 82.4 | 88.2 | 88.2 | 88.2 | 88.2 | 88.2 | 86.3 | 88.2 | 90.2 | 90.2 |
| 2. | AT4G30980 | 86.3 | 84.3 | 80.4 | 86.3 | 86.3 | 86.3 | 86.3 | 86.3 | 84.3 | 88.2 | 90.2 | 90.2 |
| 3. | AT5G58010 | 82.4 | 80.4 | 82.4 | 84.3 | 84.3 | 84.3 | 84.3 | 84.3 | 82.4 | 84.3 | 88.2 | 90.2 |
| 4. | TA12416 | 88.2 | 86.3 | 82.4 | 88.2 | 88.2 | 88.2 | 88.2 | 88.2 | 86.3 | 88.2 | 90.2 | 90.2 |
| 5. | TA5414 | 88.2 | 86.3 | 82.4 | 88.2 | 88.2 | 88.2 | 88.2 | 88.2 | 86.3 | 88.2 | 90.2 | 90.2 |
| 6. | TC205173 | 88.2 | 86.3 | 82.4 | 88.2 | 88.2 | 88.2 | 88.2 | 88.2 | 86.3 | 88.2 | 90.2 | 90.2 |
| 7. | GSVIVT00017237001 | 88.2 | 86.3 | 82.4 | 88.2 | 88.2 | 88.2 | 88.2 | 88.2 | 86.3 | 88.2 | 90.2 | 90.2 |
| 8. | TC7102 | 88.2 | 86.3 | 82.4 | 88.2 | 88.2 | 88.2 | 88.2 | 88.2 | 86.3 | 88.2 | 90.2 | 90.2 |
| 9. | TA34455 | 88.2 | 86.3 | 82.4 | 88.2 | 88.2 | 88.2 | 88.2 | 88.2 | 86.3 | 88.2 | 90.2 | 90.2 |
| 10. | TC7103 | 84.3 | 82.4 | 78.4 | 84.3 | 84.3 | 84.3 | 84.3 | 84.3 | 82.4 | 86.3 | 88.2 | 88.2 |
| 11. | TA37666 | 84.3 | 82.4 | 78.4 | 84.3 | 84.3 | 84.3 | 84.3 | 84.3 | 82.4 | 86.3 | 88.2 | 88.2 |
| 12. | TC67603 | 86.3 | 84.3 | 80.4 | 86.3 | 86.3 | 86.3 | 86.3 | 86.3 | 84.3 | 86.3 | 88.2 | 88.2 |
| 13. | Os06g08500 | 76.5 | 74.5 | 70.6 | 76.5 | 76.5 | 76.5 | 76.5 | 76.5 | 74.5 | 78.4 | 80.4 | 80.4 |
| 14. | Os02g55250 | 88.2 | 86.3 | 82.4 | 88.2 | 88.2 | 88.2 | 88.2 | 88.2 | 86.3 | 88.2 | 90.2 | 90.2 |
| 15. | Os09g25040 | 79.6 | 77.8 | 74.1 | 79.6 | 79.6 | 79.6 | 79.6 | 79.6 | 77.8 | 79.6 | 81.5 | 81.5 |
| 16. | TA2544 | 86.3 | 84.3 | 80.4 | 86.3 | 86.3 | 86.3 | 86.3 | 86.3 | 84.3 | 86.3 | 88.2 | 88.2 |
| 17. | DV982110 | 88.2 | 86.3 | 82.4 | 88.2 | 88.2 | 88.2 | 88.2 | 88.2 | 86.3 | 90.2 | 92.2 | 92.2 |
| 18. | WO051050seqID516 | 88.2 | 86.3 | 82.4 | 88.2 | 88.2 | 88.2 | 88.2 | 88.2 | 86.3 | 90.2 | 92.2 | 92.2 |
| 19. | AT4G02590 | 90.2 | 88.2 | 90.2 | 90.2 | 90.2 | 88.2 | 90.2 | 90.2 | 88.2 | 72.5 | 74.5 | 74.5 |
| 20. | TC10015_part | 90.2 | 88.2 | 90.2 | 90.2 | 90.2 | 88.2 | 90.2 | 90.2 | 88.2 | 72.5 | 74.5 | 74.5 |
| 21. | AT1G03040 | 88.2 | 86.3 | 88.2 | 88.2 | 88.2 | 86.3 | 88.2 | 88.2 | 86.3 | 72.5 | 74.5 | 74.5 |
| 22. | TA13791 | 94.1 | 92.2 | 94.1 | 92.2 | 92.2 | 90.2 | 92.2 | 92.2 | 90.2 | 74.5 | 76.5 | 76.5 |
| 23. | TC60118 | 94.1 | 92.2 | 94.1 | 92.2 | 92.2 | 90.2 | 92.2 | 92.2 | 90.2 | 74.5 | 76.5 | 76.5 |
| 24. | TC60119 | 90.2 | 90.2 | 94.1 | 88.2 | 88.2 | 90.2 | 88.2 | 88.2 | 86.3 | 74.5 | 74.5 | 74.5 |
| 25. | TC61833 | 92.2 | 90.2 | 96.1 | 90.2 | 90.2 | 92.2 | 90.2 | 90.2 | 88.2 | 74.5 | 76.5 | 76.5 |
| 26. | TA3392 | 94.1 | 92.2 | 94.1 | 92.2 | 92.2 | 90.2 | 92.2 | 92.2 | 90.2 | 74.5 | 76.5 | 76.5 |
| 27. | WO051050seqID1671 | 90.2 | 88.2 | 90.2 | 88.2 | 88.2 | 86.3 | 88.2 | 88.2 | 90.2 | 72.5 | 74.5 | 74.5 |
| 28. | Pt_TC63334 | 90.2 | 88.2 | 92.2 | 88.2 | 88.2 | 88.2 | 88.2 | 88.2 | 86.3 | 72.5 | 74.5 | 74.5 |
| 29. | TA3263 | 92.2 | 90.2 | 94.1 | 90.2 | 90.2 | 90.2 | 90.2 | 90.2 | 88.2 | 74.5 | 76.5 | 76.5 |
| 30. | Pt_scaff_II.416 | 92.2 | 90.2 | 92.2 | 90.2 | 90.2 | 88.2 | 90.2 | 90.2 | 88.2 | 72.5 | 74.5 | 74.5 |
| 31. | TA2825 | 94.1 | 92.2 | 94.1 | 92.2 | 92.2 | 90.2 | 92.2 | 92.2 | 90.2 | 74.5 | 76.5 | 76.5 |
| 32. | TC207545 | 94.1 | 92.2 | 94.1 | 92.2 | 92.2 | 90.2 | 92.2 | 92.2 | 90.2 | 74.5 | 76.5 | 76.5 |
| 33. | TC229602 | 94.1 | 92.2 | 94.1 | 92.2 | 92.2 | 90.2 | 92.2 | 92.2 | 90.2 | 74.5 | 76.5 | 76.5 |
| 34. | TA46156 | 94.1 | 92.2 | 94.1 | 92.2 | 92.2 | 90.2 | 92.2 | 92.2 | 90.2 | 74.5 | 76.5 | 76.5 |
| 35. | AK247217 | 90.2 | 88.2 | 94.1 | 88.2 | 88.2 | 90.2 | 88.2 | 88.2 | 86.3 | 72.5 | 74.5 | 74.5 |
| 36. | TA30646 | 92.2 | 90.2 | 96.1 | 90.2 | 90.2 | 92.2 | 90.2 | 90.2 | 88.2 | 74.5 | 76.5 | 76.5 |
| 37. | TC8633 | 90.2 | 88.2 | 92.2 | 92.2 | 92.2 | 92.2 | 92.2 | 92.2 | 90.2 | 76.5 | 78.4 | 78.4 |
| 38. | TC15501 | 98.0 | 96.1 | 90.2 | 92.2 | 92.2 | 90.2 | 92.2 | 92.2 | 90.2 | 80.4 | 82.4 | 82.4 |
| 39. | TA1140 | 92.2 | 92.2 | 88.2 | 94.1 | 94.1 | 92.2 | 94.1 | 94.1 | 92.2 | 78.4 | 80.4 | 80.4 |
| 40. | TA3490_part | 92.2 | 92.2 | 88.2 | 94.1 | 94.1 | 92.2 | 94.1 | 94.1 | 92.2 | 78.4 | 80.4 | 80.4 |
| 41. | CK293938 | 92.2 | 94.1 | 88.2 | 90.2 | 90.2 | 88.2 | 90.2 | 90.2 | 88.2 | 78.4 | 76.5 | 76.5 |
| 42. | TC12771 | 92.2 | 94.1 | 88.2 | 90.2 | 90.2 | 88.2 | 90.2 | 90.2 | 88.2 | 78.4 | 76.5 | 76.5 |
| 43. | TC172581 | 92.2 | 94.1 | 88.2 | 90.2 | 90.2 | 88.2 | 90.2 | 90.2 | 88.2 | 78.4 | 76.5 | 76.5 |
| 44. | TA28621 | 92.2 | 94.1 | 88.2 | 90.2 | 90.2 | 88.2 | 90.2 | 90.2 | 88.2 | 78.4 | 76.5 | 76.5 |
| 45. | TA21665 | 92.2 | 94.1 | 88.2 | 90.2 | 90.2 | 88.2 | 90.2 | 90.2 | 88.2 | 78.4 | 76.5 | 76.5 |
| 46. | TA46194 | 98.0 | 96.1 | 90.2 | 92.2 | 92.2 | 90.2 | 92.2 | 92.2 | 90.2 | 80.4 | 82.4 | 82.4 |
| 47. | DY268946 | 94.1 | 94.1 | 90.2 | 92.2 | 92.2 | 90.2 | 92.2 | 92.2 | 90.2 | 78.4 | 76.5 | 76.5 |
| 48. | CO123623 | 98.0 | 98.0 | 90.2 | 92.2 | 92.2 | 90.2 | 92.2 | 92.2 | 90.2 | 78.4 | 80.4 | 80.4 |
| 49. | Pt_scaff_70.65 | 90.2 | 92.2 | 84.3 | 86.3 | 86.3 | 84.3 | 86.3 | 86.3 | 84.3 | 74.5 | 74.5 | 74.5 |
| 50. | Pt_scaff_XIII.403 | 88.2 | 90.2 | 82.4 | 84.3 | 84.3 | 82.4 | 84.3 | 84.3 | 82.4 | 72.5 | 72.5 | 72.5 |
| 51. | TA1616 | 94.1 | 92.2 | 86.3 | 90.2 | 90.2 | 88.2 | 90.2 | 90.2 | 88.2 | 76.5 | 78.4 | 78.4 |
| 52. | TA55042 | | 96.1 | 90.2 | 92.2 | 92.2 | 90.2 | 92.2 | 92.2 | 90.2 | 78.4 | 80.4 | 80.4 |
| 53. | WO051050seqID77 | 96.1 | | 88.2 | 90.2 | 90.2 | 88.2 | 90.2 | 90.2 | 88.2 | 78.4 | 78.4 | 78.4 |
| 54. | TC3698 | 96.1 | 94.1 | 90.2 | 90.2 | 92.2 | 90.2 | 90.2 | 88.2 | 72.5 | 76.5 | 78.4 | 80.4 |
| 55. | Os03g58330 | 98.0 | 96.1 | 98.0 | | 100.0 | 98.0 | 100.0 | 100.0 | 98.0 | 78.4 | 80.4 | 80.4 |
| 56. | TA139285 | 98.0 | 96.1 | 98.0 | 100.0 | | 98.0 | 100.0 | 100.0 | 98.0 | 78.4 | 80.4 | 80.4 |
| 57. | TabHLH11 | 96.1 | 94.1 | 100.0 | 98.0 | 98.0 | | 98.0 | 98.0 | 96.1 | 78.4 | 80.4 | 80.4 |
| 58. | TC104646 | 98.0 | 96.1 | 98.0 | 100.0 | 100.0 | 98.0 | | 100.0 | 98.0 | 78.4 | 80.4 | 80.4 |
| 59. | TA126400 | 98.0 | 96.1 | 98.0 | 100.0 | 100.0 | 98.0 | 100.0 | | 98.0 | 78.4 | 80.4 | 80.4 |
| 60. | Os07g08440 | 98.0 | 96.1 | 98.0 | 100.0 | 100.0 | 98.0 | 100.0 | 100.0 | | 76.5 | 78.4 | 78.4 |
| 61. | Os02g35660 | 96.1 | 96.1 | 96.1 | 98.0 | 98.0 | 96.1 | 98.0 | 98.0 | 98.0 | | 88.2 | 88.2 |
| 62. | DT543504 | 94.1 | 92.2 | 94.1 | 96.1 | 96.1 | 94.1 | 96.1 | 96.1 | 96.1 | 98.0 | | 94.1 |
| 63. | Pt_scaff28.86 | 94.1 | 92.2 | 94.1 | 96.1 | 96.1 | 94.1 | 96.1 | 96.1 | 96.1 | 98.0 | 96.1 | |
| 64. | GSVIVT00016367001 | 96.1 | 94.1 | 96.1 | 98.0 | 98.0 | 96.1 | 98.0 | 98.0 | 98.0 | 100.0 | 98.0 | 98.0 |
| 65. | TC19278 | 68.6 | 68.6 | 68.6 | 70.6 | 70.6 | 68.6 | 70.6 | 70.6 | 70.6 | 72.5 | 70.6 | 70.6 |
| 66. | TA14134 | 94.1 | 92.2 | 92.2 | 94.1 | 94.1 | 92.2 | 94.1 | 94.1 | 94.1 | 92.2 | 94.1 | 94.1 |
| 67. | TC68930 | 98.0 | 96.1 | 98.0 | 100.0 | 100.0 | 98.0 | 100.0 | 100.0 | 100.0 | 98.0 | 98.0 | 98.0 |
| 68. | TC140470 | 94.1 | 94.1 | 94.1 | 96.1 | 96.1 | 94.1 | 96.1 | 96.1 | 96.1 | 98.0 | 96.1 | 98.0 |
| 69. | TC253044 | 94.1 | 94.1 | 94.1 | 96.1 | 96.1 | 94.1 | 96.1 | 96.1 | 96.1 | 98.0 | 96.1 | 98.0 |
| 70. | Os06g09370 | 92.2 | 92.2 | 92.2 | 94.1 | 94.1 | 92.2 | 94.1 | 94.1 | 94.1 | 96.1 | 94.1 | 96.1 |

| | | 64 | 65 | 66 | 67 | 68 | 69 | 70 |
|---|---|---|---|---|---|---|---|---|
| 1. | AT2G24260 | 92.2 | 64.7 | 88.2 | 94.1 | 86.3 | 86.3 | 88.2 |
| 2. | AT4G30980 | 92.2 | 64.7 | 90.2 | 96.1 | 86.3 | 86.3 | 88.2 |
| 3. | AT5G58010 | 92.2 | 68.6 | 86.3 | 96.1 | 82.4 | 82.4 | 84.3 |
| 4. | TA12416 | 92.2 | 64.7 | 90.2 | 94.1 | 86.3 | 86.3 | 88.2 |
| 5. | TA5414 | 92.2 | 64.7 | 90.2 | 94.1 | 86.3 | 86.3 | 88.2 |
| 6. | TC205173 | 92.2 | 64.7 | 90.2 | 94.1 | 86.3 | 86.3 | 88.2 |
| 7. | GSVIVT00017237001 | 92.2 | 64.7 | 90.2 | 94.1 | 86.3 | 86.3 | 88.2 |
| 8. | TC7102 | 92.2 | 64.7 | 90.2 | 94.1 | 86.3 | 86.3 | 88.2 |
| 9. | TA34455 | 92.2 | 64.7 | 90.2 | 94.1 | 86.3 | 86.3 | 88.2 |
| 10. | TC7103 | 90.2 | 66.7 | 90.2 | 94.1 | 84.3 | 84.3 | 86.3 |
| 11. | TA37666 | 90.2 | 66.7 | 90.2 | 94.1 | 84.3 | 84.3 | 86.3 |
| 12. | TC67603 | 90.2 | 64.7 | 88.2 | 92.2 | 84.3 | 84.3 | 86.3 |
| 13. | Os06g08500 | 82.4 | 64.7 | 82.4 | 86.3 | 76.5 | 76.5 | 78.4 |
| 14. | Os02g55250 | 92.2 | 64.7 | 90.2 | 94.1 | 86.3 | 86.3 | 88.2 |
| 15. | Os09g25040 | 83.3 | 57.4 | 81.5 | 85.2 | 77.8 | 77.8 | 79.6 |
| 16. | TA2544 | 90.2 | 62.7 | 88.2 | 92.2 | 84.3 | 84.3 | 86.3 |
| 17. | DV982110 | 94.1 | 66.7 | 88.2 | 96.1 | 88.2 | 88.2 | 90.2 |
| 18. | WO051050seqID516 | 94.1 | 66.7 | 88.2 | 96.1 | 88.2 | 88.2 | 90.2 |
| 19. | AT4G02590 | 76.5 | 49.0 | 74.5 | 78.4 | 70.6 | 70.6 | 72.5 |
| 20. | TC10015_part | 76.5 | 49.0 | 74.5 | 78.4 | 70.6 | 70.6 | 72.5 |
| 21. | AT1G03040 | 76.5 | 49.0 | 76.5 | 80.4 | 70.6 | 70.6 | 72.5 |
| 22. | TA13791 | 78.4 | 51.0 | 76.5 | 80.4 | 72.5 | 72.5 | 74.5 |
| 23. | TC60118 | 78.4 | 51.0 | 76.5 | 80.4 | 72.5 | 72.5 | 74.5 |
| 24. | TC60119 | 76.5 | 51.0 | 74.5 | 78.4 | 72.5 | 72.5 | 74.5 |
| 25. | TC61833 | 78.4 | 51.0 | 76.5 | 80.4 | 72.5 | 72.5 | 74.5 |
| 26. | TA3392 | 78.4 | 51.0 | 76.5 | 80.4 | 72.5 | 72.5 | 74.5 |
| 27. | WO051050seqID1671 | 76.5 | 49.0 | 74.5 | 78.4 | 74.5 | 74.5 | 74.5 |
| 28. | Pt_TC63334 | 76.5 | 49.0 | 74.5 | 78.4 | 70.6 | 70.6 | 72.5 |
| 29. | TA3263 | 78.4 | 51.0 | 76.5 | 80.4 | 72.5 | 72.5 | 74.5 |
| 30. | Pt_scaff_II.416 | 76.5 | 49.0 | 74.5 | 78.4 | 70.6 | 70.6 | 72.5 |
| 31. | TA2825 | 78.4 | 51.0 | 76.5 | 80.4 | 72.5 | 72.5 | 74.5 |
| 32. | TC207545 | 78.4 | 51.0 | 76.5 | 80.4 | 72.5 | 72.5 | 74.5 |
| 33. | TC229602 | 78.4 | 51.0 | 76.5 | 80.4 | 72.5 | 72.5 | 74.5 |
| 34. | TA46156 | 78.4 | 51.0 | 76.5 | 80.4 | 72.5 | 72.5 | 74.5 |
| 35. | AK247217 | 76.5 | 49.0 | 74.5 | 78.4 | 70.6 | 70.6 | 72.5 |
| 36. | TA30646 | 78.4 | 51.0 | 76.5 | 80.4 | 72.5 | 72.5 | 74.5 |
| 37. | TC8633 | 80.4 | 52.9 | 78.4 | 82.4 | 74.5 | 74.5 | 76.5 |
| 38. | TC15501 | 84.3 | 56.9 | 78.4 | 86.3 | 78.4 | 78.4 | 80.4 |
| 39. | TA1140 | 82.4 | 54.9 | 78.4 | 84.3 | 76.5 | 76.5 | 78.4 |
| 40. | TA3490_part | 82.4 | 54.9 | 78.4 | 84.3 | 76.5 | 76.5 | 78.4 |
| 41. | CK293938 | 78.4 | 52.9 | 76.5 | 80.4 | 76.5 | 76.5 | 78.4 |
| 42. | TC12771 | 78.4 | 52.9 | 76.5 | 80.4 | 76.5 | 76.5 | 78.4 |
| 43. | TC172581 | 78.4 | 52.9 | 76.5 | 80.4 | 76.5 | 76.5 | 78.4 |
| 44. | TA28621 | 78.4 | 52.9 | 76.5 | 80.4 | 76.5 | 76.5 | 78.4 |
| 45. | TA21665 | 78.4 | 52.9 | 76.5 | 80.4 | 76.5 | 76.5 | 78.4 |
| 46. | TA46194 | 84.3 | 56.9 | 78.4 | 86.3 | 78.4 | 78.4 | 80.4 |
| 47. | DY268946 | 78.4 | 52.9 | 76.5 | 80.4 | 72.5 | 72.5 | 74.5 |
| 48. | CO123623 | 82.4 | 54.9 | 78.4 | 84.3 | 76.5 | 76.5 | 78.4 |
| 49. | Pt_scaff_70.65 | 76.5 | 52.9 | 74.5 | 78.4 | 72.5 | 72.5 | 74.5 |
| 50. | Pt_scaff_XIII.403 | 74.5 | 51.0 | 72.5 | 76.5 | 70.6 | 70.6 | 72.5 |
| 51. | TA1616 | 80.4 | 52.9 | 78.4 | 82.4 | 74.5 | 74.5 | 76.5 |
| 52. | TA55042 | 82.4 | 54.9 | 78.4 | 84.3 | 76.5 | 76.5 | 78.4 |
| 53. | WO051050seqID77 | 80.4 | 54.9 | 76.5 | 82.4 | 76.5 | 76.5 | 78.4 |
| 54. | TC3698 | 52.9 | 74.5 | 82.4 | 70.6 | 70.6 | 72.5 | |
| 55. | Os03g58330 | 82.4 | 54.9 | 78.4 | 84.3 | 76.5 | 76.5 | 78.4 |
| 56. | TA139285 | 82.4 | 54.9 | 78.4 | 84.3 | 76.5 | 76.5 | 78.4 |
| 57. | TabHLH11 | 82.4 | 54.9 | 78.4 | 84.3 | 76.5 | 76.5 | 78.4 |
| 58. | TC104646 | 82.4 | 54.9 | 78.4 | 84.3 | 76.5 | 76.5 | 78.4 |
| 59. | TA126400 | 82.4 | 54.9 | 78.4 | 84.3 | 76.5 | 76.5 | 78.4 |
| 60. | Os07g08440 | 80.4 | 52.9 | 76.5 | 82.4 | 78.4 | 78.4 | 78.4 |
| 61. | Os02g35660 | 90.2 | 64.7 | 82.4 | 88.2 | 88.2 | 88.2 | 90.2 |
| 62. | DT543504 | 96.1 | 66.7 | 82.4 | 92.2 | 88.2 | 88.2 | 90.2 |
| 63. | Pt_scaff28.86 | 98.0 | 68.6 | 82.4 | 94.1 | 90.2 | 90.2 | 92.2 |
| 64. | GSVIVT00016367001 | | 70.6 | 84.3 | 96.1 | 90.2 | 90.2 | 92.2 |
| 65. | TC19278 | 72.5 | | 62.7 | 68.6 | 62.7 | 62.7 | 64.7 |
| 66. | TA14134 | 96.1 | 72.5 | | 88.2 | 78.4 | 78.4 | 80.4 |
| 67. | TC68930 | 100.0 | 72.5 | 96.1 | | 86.3 | 86.3 | 88.2 |
| 68. | TC140470 | 98.0 | 70.6 | 94.1 | 98.0 | 100.0 | 96.1 | |
| 69. | TC253044 | 98.0 | 70.6 | 94.1 | 98.0 | 100.0 | | 96.1 |
| 70. | Os06g09370 | 96.1 | 68.6 | 92.2 | 96.1 | 98.0 | 98.0 | |

### Example 46

### Identification of Domains Comprised in Polypeptide Sequences Useful in Performing the Methods of the Invention

The Integrated Resource of Protein Families, Domains and Sites (interPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 245 are presented in Table E4.

TABLE E4
InterPro scan results (major accession numbers) of the polypeptide
sequence as represented by SEQ ID NO: 245.

| | | | Amino |
|---|---|---|---|
| | | | acid coordinates |
| | Accession | | on SEQ ID NO 245; |
| Database | number | Accession name | e-value |
| InterPro | IPR001092 | Basic helix-loop-helix | |
| | | dimerisation region | |
| | | bHLH | |
| HMMPfam | PF00010.14 | Helix-loop-helix | T[127-176] 1.6e−06 |
| | | DNA-binding domain | |
| HMMSmart | SM00353 | no description | T[132-181] 1.5e−09 |
| ProfileScan | PS50888 | HLH | T[120-176] 12.451 |
| InterPro | IPR011598 | Helix-loop-helix | |
| | | DNA-binding | |
| superfamily | SSF47459 | Helix-loop-helix | T[122-195] 1.8e−14 |
| | | DNA-binding domain | |

### Example 47

### Topology Prediction of the Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 245 are presented Table E5. The “plant” organism group has been selected, no cutoffs defined, and the predicted length of the transit peptide requested. The subcellular localization of the polypeptide sequence as represented by SEQ ID NO: 245 may be the cytoplasm or nucleus, no transit peptide is predicted.

TABLE E5
TargetP 1.1 analysis of the polypeptide sequence as
represented by SEQ ID NO: 245

| | |
|---|---|
| Length (AA) | 281 |
| Chloroplastic transit peptide | 0.094 |
| Mitochondrial transit peptide | 0.166 |
| Secretory pathway signal peptide | 0.034 |
| Other subcellular targeting | 0.855 |
| Predicted Location | / |
| Reliability class | 2 |
| Predicted transit peptide length | / |

When analysed with PLOC (Park and Kanehisa, Bioinformatics, 19, 1656-1663, 2003) or with PSORT (URL: psort.org), the protein is predicted to have a nuclear localisation.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark;

### Example 48

### Functional Assay for the bHLH11-Like Polypeptide

A DNA-binding assay for AtbHLH6 is provided in Dombrecht et al. (2007), this approach can be used for any bHLH11-like protein. Briefly, a 1000-bp fragment of the AtbHLH6 coding sequence encompassing codons 285 to 623 was amplified from genomic DNA and cloned into the Nhet-BamHI-digested pTacLCELD6•His vector (Xue, Plant J. 41, 638-649, 2005). The resulting construct encoded the last 338 amino acids of AtbHLH6 (including the bHLH region) in-frame with the reporter protein CelD and a 6×His tag. Correct amplification and cloning were verified by DNA sequencing.

Determination of the consensus sequence of the MYC2 DNA binding motif and the relative binding affinity of these sites was done according to Xue (2005), wherein a fusion of a DNA-binding protein (DBP) to 6His-tagged cellulase D (GELD) serves both as a means for affinity purification of DBP-DNA complex in the selection of binding sites from a pool of biotinylated random-sequence oligonucleotides and as a reporter for measurement of DNA-binding activity.

For the selection of binding-sites using cellulose paper as an affinity matrix, DBP-CELD was incubated at room temperature for 1 h with 20 ng of a biotin-labelled Bio-RS-Oligo in 40 μl of binding/washing buffer (described above) containing 1 mM EDTA, 0.25 μg μl⁻¹ poly d(AC-TG), 1 mg ml⁻¹ BSA and 10% glycerol. An appropriate amount of crude DBP-GELD used for binding-site selection was that achieving 20-30% relative cellulose binding efficiency (percentage cellulose activity bound to cellulose after washing). DBP-CELD/Bio-RS-Oligo mixtures were transferred to 96-well microplate wells containing Whatman 1 filter paper (4 mm) which was pre-soaked with 10 μl blocking solution [binding/washing buffer containing 0.5 μg μl⁻¹ poly d(AC-TG) and 10% glycerol]. After incubation at 0° C. for 1 hr with gentle shaking, the cellulose paper was washed six times with binding/washing buffer containing 1 mM EDTA and 0.1 mg ml⁻¹ BSA. The use of extensive washing in the target site selection was based on observations of the relatively high stability of DBP-oligonucleotide complex immobilized on solid matrix. DBP-CELD carrying target binding sites were eluted at 40° C. for 15 min with 40 μl of cellulase eluting buffer [10 mM HEPES, pH7, 50 mM KCl, 0.2 mM EDTA, 4 mM cellobiose, 0.05 mg ml⁻¹ BSA and 10 μg ml⁻¹ of a sense sequence-specific primer SP-S]. The eluate was used for PCR amplification of selected oligonucleotides. The PCR product (0.1 μl) without purification was used for the next round of site selection.

Alternatively, 6-His tagged DBP-GELD (10-25 μg crude protein) was incubated in 60 μl of PNT buffer (50 mM sodium phosphate, pH8.0, 300 mM NaCl and 0.05% Tween 20) containing 10 mM imidazole and 350 μg Ni-NTA magnetic agarose beads (Qiagen) at room temperature for 45-60 min with gentle shaking in a micro-tube mixer (Torry Seiko Co., Tokyo, Japan). The Ni-NTA magnetic beads were collected at the side of the tubes by placing tubes in a 12-tube magnet (Qiagen). Unbound proteins were removed by washing twice with 150-200 μl of PNT containing 20 mM imidazole and once with 60 μl of binding/washing buffer containing 2 mM MgCl₂, 1 mg ml⁻¹ BSA and 10% glycerol. Washed beads were suspended in 40 μl of binding/washing buffer containing 2 mM MgCl₂, 0.25 μg μl⁻¹ poly d(AC-TG), 1 mg ml⁻¹ BSA, 0.0025% Nonidet P-40, 10% glycerol and biotin-labelled oligonucleotides (50 ng Bio-RS-Oligo or 1 μl of PCR product amplified from previously selected oligonucleotides). The suspension was incubated at room temperature for 1.5 h with gentle shaking in the micro-tube mixer. After washing four times (3-4 min each washing) with 150-200 μl of binding/washing buffer containing 2 mM MgCl₂, 0.1 mg ml⁻¹ BSA and 0.0025% Nonidet P-40, the beads carrying target site-bound BDP-GELD were resuspended in 8 μl of 5 mM Tris-Cl (pH 8.0)/0.5 mM EDTA containing 5 μg ml⁻¹ of a sense sequence-specific primer (SP-S) and the suspension was transferred to a clean tube and used for PCR amplification of selected oligonucleotides. The PCR product (1 μl) without purification was used for the next round of site selection.

For EMSA assays, 6•His-tagged DBP-GELD proteins were purified using Ni-NTA magnetic agarose beads using a high stringent binding buffer (50 mMsodium phosphate, pH8.0, 1 MNaCl, 10% glycerol, 1% Tween 20 and 10 mM imidazole) and the rest of the procedure followed the manufacturer's instruction. Double-stranded synthetic oligonucleotides (30-45 fmol) labelled with digoxigenin at the 3′-end were incubated with a purified DBP (30-75 ng) in 15 μl of binding buffer [25 mM HEPES/KOH, pH 7.0, 50 mM KCl, 0.5 mM DTT, 2 mM MgCl₂, 0.2 μg μl⁻¹ poly d(AC-TG), 0.3 mg ml⁻¹ BSA and 10% glycerol]. After incubation at room temperature for 30 min, DBP/DNA complexes were separated from free probes on a 6% polyacrylamide gel in a 40-mM Tris-acetate buffer (pH 7.5) containing 5 mM Na acetate, 0.5 mM EDTA and 5% glycerol. The DBP/DNA complexes and free probes in the gels after electrophoresis were transferred to a Hybond N [symbol] membrane. Alkaline phosphatase-conjugated anti-digoxigenin antibody and a chemiluminescent substrate, COP-Star (Roche Diagnostics) was used for detection of digoxigenin according to the manufacturer's instructions.

Further details are provided in Xue (2005).

### Example 49

### Cloning of the Nucleic Acid Sequence Used in the Methods of the Invention

The nucleic acid sequence used in the methods of the invention and comprising SEQ ID NO: 244 was amplified by PCR using as template a custom-made Triticum aestivum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm009718 (SEQ ID NO: 254; sense, start codon in bold):

5′-ggggacaagtttgtacaaaaaagcaggcttaaacaatggcgggg
canccg-3′

and prm009719 (SEQ ID NO: 255; reverse, complementary):

5′-ggggaccactttgtacaagaaagctgggtatgggtgttgcagct
gctgtt-3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pbHLH11-like. Plasmid pDONR201 was purchased from Invitrogen, as part of the Gateway® technology.

The entry clone comprising SEQ ID NO: 244 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 256) for constitutive expression was located upstream of this Gateway cassette.

After the LR recombination step, the resulting expression vector pGOS2::bHLH11-like (FIG. 23) was transformed into Agrobacterium strain LBA4044 according to methods well known in the art.

### Example 50

### Plant Transformation

Transformation of plants was carried out according to the procedure outlined in Example 7.

### Example 51

### Phenotypic Evaluation Procedure

### 51.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are watered at regular intervals to ensure that water and nutrients are not limiting to satisfy plant needs to complete growth and development.

From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approach the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution are used during the first two weeks after transplanting the plantlets in the greenhouse. After the first two weeks, 25 mM of salt (NaCl) is added to the nutrient solution, until the plants are harvested. Seed-related parameters are then measured.

### 51.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

### 51.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 52

### Results of the Phenotypic Evaluation of the Transgenic Plants

The results of the evaluation of transgenic rice plants expressing a bHLH11-like nucleic acid under non-stress conditions are presented below. An increase of more than 5% in at least 2 lines was observed for total seed yield, number of filled seeds, fill rate, harvest index, number of first panicles and of at least 3% for thousand kernel weight. Data on the overall increase (measured over all tested lines) for each parameter are presented in Table E6:

TABLE E6

| Parameter | Overall increase (%) |
|---|---|
| Total weight of seeds (total seed yield) | 20 |
| Number of filled seeds | 20 |
| Fill rate | 18 |
| Harvest Index | 21 |

### VI. ASR (Abscisic Acid-, Stress-, and Ripening-Induced) Protein

### Example 53

### Identification of Sequences Related to the ASR Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program was used to find regions of local similarity between sequences by comparing nucleic acid or polypeptide sequences to sequence databases and by calculating the statistical significance of matches. For example, the polypeptide encoded by the nucleic acid used in the present invention were used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length.

Table F1 provides a list of nucleic acid and polypeptide sequences related to the nucleic acid sequence used in the methods of the present invention.

TABLE F1
Examples of ASR nucleic acids and polypeptides.

| | | Nucleic acid | Protein |
|---|---|---|---|
| Name | Plant origin | SEQ ID NO: | SEQ ID NO: |
| Orysa_ASR_1 | Oryza sativa | 396 | 397 |
| Zeama_ASR_1 | Zea mays | 401 | 402 |
| Zeama_ASR_2 | Zea mays | 403 | 404 |
| Zeama_ASR_3 | Zea mays | 405 | 406 |
| Zeama_ASR_4 | Zea mays | 407 | 408 |
| Zeama_ASR_5 | Zea mays | 409 | 410 |
| Zeama_ASR_6 | Zea mays | 411 | 412 |
| Zeama_ASR_7 | Zea mays | 413 | 414 |
| Zeama_ASR_8 | Zea mays | 415 | 416 |
| Zeama_ASR_9 | Zea mays | 417 | 418 |
| Lyces_ASR_1 | Lycopersicon esculentum | 419 | 420 |
| Lyces_ASR_2 | Lilium longiflorum | 421 | 422 |
| Sacof_ASR_1 | Saccharum officinarum | 423 | 424 |
| Zeama_ASR_10 | Zea mays | 425 | 426 |

### Example 54

### Alignment of ASR Polypeptide Sequences

Alignment of polypeptide sequences is performed using the AlignX programme from the Vector NTI package (Invitrogen) which is based on the popular Cluster W algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0,1 and the selected weight matrix is Blosum 62 (if polypeptides are aligned). Minor manual editing may be done to further optimise the alignment.

A phylogenetic tree of GRP polypeptides is constructed using a neighbour-joining clustering algorithm as provided in the AlignX programme from Vector NTI (Invitrogen).

### Example 55

### Calculation of Global Percentage Identity Between ASR Polypeptide Sequences Useful in Performing the Methods of the Invention

Global percentages of similarity and identity between full length polypeptide sequences useful in performing the methods of the invention are determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

A MATGAT table for local alignment of a specific domain, or data on % identity/similarity between specific domains may also be generated.

### Example 56

### Identification of Domains Comprised in ASR Polypeptide Sequences Useful in Performing the Methods of the Invention

The Integrated Resource of Protein Families, Domains and Sites (InterPro) database is an integrated interlace for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The protein sequences representing the GRP are used as query to search the InterPro database.

### Example 57

### Topology Prediction of ASR Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained by the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The protein sequences representing the GRP are used to query TargetP 1.1. The “plant” organism group is selected, no cutoffs defined, and the predicted length of the transit peptide requested.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 58

### Cloning of an ASR Nucleic Acid Sequence Used in the Methods of the Invention

### Cloning of SEQ ID NO: 396:

The nucleic acid sequence SEQ ID NO: 396 used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm06442 (SEQ ID NO: 399; sense, start codon in bold):
5′-ggggacaagtttgtacaaaaaagcaggcttaaacaatggcggaggagaagca-3′
   and prm06443 (SEQ ID NO: 400; reverse, complementary):
5′-ggggaccactttgtacaagaaagctgggtcggcgacgttgtgatga-3%
   which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 396 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 398) for seed specific expression was located upstream of this Gateway cassette.

After the LR recombination step, the resulting expression vector pGOS2::GRP (FIG. 25) was transformed into Agrobacterium strain LBA4044 according to methods well known in the art.

### Example 59

### Plant Transformation

Transformation of plants was carried out according to the procedure outlined in Example 7

### Example 60

### Phenotypic Evaluation Procedure

### 60.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event.

From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions. Plants grown under non-stress conditions are supplied with water at regular intervals to ensure that water and nutrients are not limiting to satisfy plant needs to complete growth and development.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### 60.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis is performed. This is useful to check consistency of the effects over the two experiments, and if this is the case, to accumulate evidence from both experiments in order to increase confidence in the conclusion. The method used is a mixed-model approach that takes into account the multilevel structure of the data (i.e. experiment—event—segregants). P values were obtained by comparing likelihood ratio test to chi square distributions.

### 60.3 Parameters Measured

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 61

### Results of the Phenotypic Evaluation of the Transgenic Plants

The transgenic rice plants expressing the GRP nucleic acid represented by SEQ ID NO: 396 under control of the GOS2 promoter showed an increase of more than 5% for total weight of seeds, number of filled seeds, and harvest index, when grown under non-stress conditions.

### VII. Squamosa Promoter Binding Protein-Like 11 (SPL11)

### Example 62

### Identification of Sequences Related to the SPL 11 Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI). The sequences search was also carried out in other public and proprietary databases. Sequence search tools were used, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table G1 provides a list of nucleic acid sequences related to the nucleic acid sequence used in the methods of the present invention. Polypeptides with an accession extended by a dot and one digit represent splice variants.

TABLE G1
Examples of SPL11 nucleic acids and polypeptides

| | | | Nucleic acid | Protein SEQ |
|---|---|---|---|---|
| Name | Alias | Plant origin | SEQ ID NO: | ID NO: |
| Arath_SPL11a | At1g27360 | Arabidopsis thaliana | 427 | 428 |
| Arath_SPL11aa | NM_202191 | Arabidopsis thaliana | 429 | 428 |
| | (splicing variant) | | | |
| Arath_SPL11aaa | NM_001084135 | Arabidopsis thaliana | 430 | 428 |
| Arath_SPL11aaaa | SEQID NO: 1 Variant | Synthetic | 431 | 428 |
| | MiR156 insensitive | | | |
| Arath_SPL11b | At1g27370 | Arabidopsis thaliana | 432 | 433 |
| Arath_SPL11c | At5g43270 | Arabidopsis thaliana | 434 | 435 |
| Orysa_SPL11a | LOC_Os02g04680 | Oryza sativa | 436 | 437 |
| Orysa_SPL11b | LOC_Os06g49010 | Oryza sativa | 438 | 439 |
| Popth_SPL11a | Populus SPL11a | Populus Species | 440 | 441 |
| Popth_SPL11b | Populus SPL11b | Populus Species | 442 | 443 |
| Popth_SPL11c | Populus SPL11c | Populus Species | 444 | 445 |
| Brara_SPL11a | Br_AC189413 | Brassica rapa | 446 | 447 |
| Zeama_SPL11a | ZM_60752693 | Zea mays | 448 | 449 |
| Zeama_SPL11b | ZM_SPL11b | Zea mays | 450 | 451 |
| Triae_SPL11a | Ta_SPL11a | Triticum aestivum | 452 | 453 |
| Vitvi_SPL11a | Vv_SPL11a | Vitis vinifera | 454 | 455 |

### Example 63

### Alignment of SPL11 Polypeptide Sequences

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0,1 and the selected weight matrix is Blosum 62 (if polypeptides are aligned). Minor manual editing was done to further optimise the alignment. A consensus sequence comprising highly conserved representative amino acids at each position is given; blanks in between given amino acids in the consensus sequence represent any amino acid. High sequence conservation among SPL11 polypeptides is observed mostly along the SBP domain of the polypeptides. The position of other conserved sequence motifs outside of the SBP domain herein represented by Motif 1 to Motif 4 (SEQ ID NO: 466 to SEQ ID NO: 472) is indicated over the consensus sequence.

FIG. 28 provides an alignment of representative SPL11 polypeptides.

### Example 64

### Calculation of Global Percentage Identity Between SPL11 Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table G2 for the global similarity and identity over the full length of the polypeptide sequences and Table G3 for the similarity on the SBP domain. Percentage identity is given above the diagonal in bold and percentage similarity is given below the diagonal (normal face).

The percentage identity between the SPL11 polypeptide sequences useful in performing the methods of the invention can be as low as 20% amino acid identity compared to SEQ ID NO: 428. Typically, SPL11 polypeptides have a global (along the sequence of the entire polypeptide) sequence identity in the range of 40%.

The percentage identity between SBP domains comprised in SPL11 polypeptide sequences useful in performing the methods of the invention can be as low as 30% amino acid identity compared to SEQ ID NO: 456 (SBP domain comprised in Arath_SPL11a or SEQ ID NO: 428). The SBP domains comprised in the SPL11 polypeptides given in Table G1 have a sequence identity in the range of 31 to 93.6%.

TABLE G2
MatGAT results for global similarity and identity over the full length of the
polypeptide sequences.

| Name sequence | 1 | 2 | 3 | 4 | 5 | 6 | 7 | 8 | 9 | 10 | 11 | 12 | 13 |
|---|---|---|---|---|---|---|---|---|---|---|---|---|---|
| 1. Arath_SPL11a | | 75.3 | 37.2 | 31.2 | 30.9 | 38.4 | 34.3 | 29.9 | 31 | 27.5 | 30.7 | 31.4 | 31.5 |
| 2. Arath_SPL11b | 82.3 | | 36.6 | 33.2 | 32.1 | 37.9 | 36.4 | 29.7 | 31.3 | 29.5 | 30.7 | 31.2 | 33.2 |
| 3. Arath_SPL11c | 52.5 | 52 | | 33.6 | 34.9 | 40.4 | 38.6 | 33.7 | 48.5 | 32.9 | 31.8 | 32.4 | 34.4 |
| 4. Orysa_SPL11a | 46.5 | 44.6 | 46.7 | | 54.3 | 36.6 | 38.9 | 38.1 | 25.6 | 46.9 | 49.2 | 66.2 | 34.5 |
| 5. Orysa_SPL11b | 43.8 | 43.4 | 47.8 | 68.2 | | 35.4 | 36.7 | 38.4 | 25.2 | 44.3 | 55.1 | 55.2 | 32.6 |
| 6. Popth_SPL11a | 54.5 | 52 | 54.7 | 49 | 49.1 | | 66.5 | 35.4 | 36.4 | 30 | 32.3 | 35.4 | 44.1 |
| 7. Popth_SPL11b | 46.7 | 48.6 | 52.3 | 55.9 | 53.9 | 71.2 | | 37.7 | 32.7 | 30.7 | 34.7 | 37 | 42.5 |
| 8. Popth_SPL11c | 44.7 | 44.7 | 50.4 | 55.9 | 55.6 | 48.9 | 55.7 | | 25.8 | 33.3 | 34.2 | 35.7 | 37.6 |
| 9. Brara_SPL11a | 41.7 | 42.4 | 55.6 | 34.1 | 34.3 | 46.6 | 40 | 35.2 | | 22.2 | 24.1 | 24.8 | 29.4 |
| 10. Zeama_SPL11a | 42.1 | 42.5 | 44.6 | 61.4 | 58.3 | 42.7 | 45.9 | 51 | 31.5 | | 41.6 | 49.4 | 28.6 |
| 11. Zeama_SPL11b | 44.2 | 44.4 | 46.7 | 60.6 | 66.7 | 47.6 | 50.5 | 50.4 | 35.1 | 53.1 | | 49.5 | 32.1 |
| 12. Triae_SPL11a | 44 | 44 | 46.3 | 77 | 69.5 | 48 | 52 | 53.9 | 33.2 | 62.2 | 61.1 | | 32.8 |
| 13. Vitvi_SPL11a | 44.6 | 43.5 | 47.2 | 50.2 | 50.4 | 53.8 | 55.8 | 55.8 | 36.7 | 46.4 | 46.4 | 50.2 | |

TABLE G3
MatGAT results for similarity and identity over the SBP domain of polypeptide
sequences.

| Name sequence | 1 | 2 | 3 | 4 | 5 | 6 | 7 | 8 | 9 | 10 | 11 | 12 |
|---|---|---|---|---|---|---|---|---|---|---|---|---|
| 1. Arath_SPL11a | | 93.7 | 77.2 | 73.4 | 75.9 | 58.3 | 62.2 | 49.5 | 46.6 | 73.4 | 73.4 | 68.2 |
| 2. Arath_SPL11b | 96.2 | | 79.7 | 73.4 | 73.4 | 59.3 | 63.3 | 49.5 | 48.9 | 75.9 | 77.2 | 71.8 |
| 3. Arath_SPL11c | 86.1 | 88.6 | | 81 | 77.2 | 63 | 66.3 | 54.2 | 59.1 | 81 | 78.5 | 74.1 |
| 4. Orysa_SPL11a | 84.8 | 86.1 | 89.9 | | 84.8 | 61.5 | 66.3 | 53.3 | 50 | 88.6 | 83.5 | 80 |
| 5. Orysa_SPL11b | 88.6 | 91.1 | 91.1 | 91.1 | | 59.3 | 64.3 | 50.5 | 47.7 | 87.3 | 83.5 | 78.8 |
| 6. Popth_SPL11a | 64.8 | 67.6 | 68.5 | 65.7 | 66.7 | | 60.6 | 52 | 45.4 | 62.4 | 57.4 | 60.2 |
| 7. Popth_SPL11b | 69.4 | 72.4 | 72.4 | 70.4 | 72.4 | 71.3 | | 45.2 | 41.3 | 66.3 | 62.2 | 61.5 |
| 8. Popth_SPL11c | 63.3 | 64.3 | 65.3 | 64.3 | 66.3 | 66.7 | 65.3 | | 31 | 53.3 | 50.5 | 56.1 |
| 9. Brara_SPL11a | 62 | 64.6 | 68.4 | 63.3 | 64.6 | 56.5 | 55.1 | 43.9 | | 51.1 | 51.2 | 46.8 |
| 10. Zeama_SPL11a | 86.1 | 88.6 | 91.1 | 93.7 | 96.2 | 66.7 | 70.4 | 67.3 | 63.3 | | 88.6 | 84.7 |
| 11. Zeama_SPL11b | 88.6 | 91.1 | 89.9 | 88.6 | 93.7 | 63.9 | 69.4 | 64.3 | 64.6 | 92.4 | | 90.6 |
| 12. Triae_SPL11a | 82.4 | 84.7 | 84.7 | 84.7 | 88.2 | 67.6 | 72.4 | 71.4 | 60 | 88.2 | 90.6 | |
| 13. Vitvi_SPL11a | 90.1 | 93.8 | 90.1 | 87.7 | 90.1 | 70.4 | 75.5 | 67.3 | 64.2 | 88.9 | 87.7 | 87.1 |

### Example 65

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pram is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

Table G4 shows the settings (Gathering cut off, trusted cut off and noise cut off) as described in the Pfam database that were used to build the HMMs_fs for SBP different domains.

TABLE G4
HMMs_build information in domain database Pfam Version 21.0

| HMMER | Pfam_ls | |
|---|---|---|
| build information | [Download HMM] | Pfam_fs [Download HMM] |
| Gathering cutoff | 25.0 25.0; | 25.0 25.0 |
| Trusted cutoff | 41.4 41.4; | 26.2 54.1 |
| Noise cutoff | 20.8 20.8; | 18.0 11.2 |
| Build method of | hmmbuild -F | hmmbuild -f -F HMM_fs SEED |
| HMM | HMM_ls SEED | hmmcalibrate --seed 0 HMM_fs |
| | hmmcalibrate --seed | |
| | 0 HMM_ls | |

The results of the Pfam scan for representative SPL11 polypeptides of plant origin are presented in Table G5. The amino acid coordinates for SBP domain in the sequence of reference is indicated in the corresponding column. The E-value of the alignment is also given.

TABLE G5
Pfam scan results for the SBP domain (Pfam Reference PF03110)
of representative SPL11 polypeptides as perform on Pfam Version 21.0.

| | Amino acid coordinates | e-value of the |
|---|---|---|
| Name sequence | of the SBP domain | alignment |
| Arath_SPL11a | 174-252 | 1.10E−50 |
| Arath_SPL11b | 175-253 | 1.61E−47 |
| Arath_SPL11c | 168-246 | 1.90E−52 |
| Orysa_SPL11a | 181-259 | 5.10E−50 |
| Orysa_SPL11b | 179-257 | 3.90E−47 |
| Popth_SPL11a | 170-277 | 170-277 |
| Popth_SPL11b | 180-277 | 1.50E−41 |
| Popth_SPL11c | 171-249 | 7.80E−52 |
| Brara_SPL11a | 180-279 | 5.20E−44 |
| Zeama_SPL11a | 161-239 | 2.20E−51 |
| Zeama_SPL11b | 159-237 | 1.10E−48 |
| Triae_SPL11a | 184-262 | 2.90E−52 |
| Vitvi_SPL11a | 171-249 | 7.80E−52 |

### Example 66

### Cloning of the Nucleic Acid Sequence Used in the Methods of the Invention

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. An upstream and downstream primer as represented by SEQ ID NO: 473 and SEQ ID NO: 474 respectively were used to amplify by PCR (Polymerase Chain Reaction) the coding region of a SPL11 nucleic acid as represented by SEQ ID NO: 427. The primers include the AttB sites for Gateway recombination to facilitate the cloning of the amplified PCR DNA fragment into a Gateway cloning vector.

The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pSPL11. Plasmid pDONR201 was purchased from Invitrogen, as part of the Gateway® technology.

The entry clone comprising SEQ ID NO: 427 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 476) for constitutive expression was located upstream of this Gateway cassette in the vector pGOS2::SPL11. The SPL11 coding sequence was also cloned in an expression vector comprising a rice WSI18 promoter (SEQ ID NO: 477) for seed specific (ABA inducible) expression (pWSI18::SPL11).

After the LR recombination step, the resulting expression vectors pGOS2::SPL11 and pWSI18::SPL11 (FIG. 31) were transformed into Agrobacterium strain LBA4044 according to methods well known in the art.

### Example 67

### Plant Transformation

Transformation of plants was carried out according to the procedure outlined in Example 7

### Example 68

### Phenotypic Evaluation Procedure

### 68.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are supplied with water at regular intervals to ensure that water and nutrients are not limiting to satisfy plant needs to complete growth and development.

Four T1 events were further evaluated in the T2 generation following the same evaluation procedure as for the T1 generation but with more individuals per event. From the stage of sowing until the stage of maturity the plants were passed several times through a digital imaging cabinet. At each time digital images (2048×1536 pixels, 16 million colours) were taken of each plant from at least 6 different angles.

### Drought Screen

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### 68.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value to a gene effect, meaning that it is not only the mere presence or position of the gene that is causing the differences in phenotype.

When two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P values were obtained by comparing likelihood ratio test to chi square distributions.

### 68.3 Parameters Measured

### Biomass-Related Parameter Measurement

From the stage of sowing until the stage of maturity the plants were passed several times through a digital imaging cabinet. At each time digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time from the different angles and was converted to a physical surface value expressed in square mm by calibration. Experiments show that the aboveground plant area measured this way correlates with the biomass of plant parts above ground. The above ground area is the area measured at the time at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Emergence vigour was determined by counting the total number of pixels from aboveground plant parts discriminated from the background. This value was averaged for the pictures taken on the same time from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 69

### Results of the Phenotypic Evaluation of the Transgenic Plants

The results of the evaluation of transgenic rice plants expressing a SPL11 nucleic acid corresponding to the construct pGOS2::SPL11 and pWSI18::SPL11 whether under drought stress or non-stress conditions are given herein. An increase of at least 5% was observed for one or more of the following parameters, emergence vigour (seedling early vigour), total seed yield (seed weight), the seed fill rate (seed filling rate), the number of filled seeds, the number of flowers (seeds) per panicle and the harvest index, and of at least 3% for thousand-kernel (1000-seed) weight in the transgenic plants of when compared to the control nullizygote plants in at least one of the growth conditions.

1-169. (canceled)

170. A method for enhancing yield-related traits in plants relative to control plants, comprising modulating expression in a plant of a nucleic acid encoding an LBD polypeptide, wherein said LBD polypeptide comprises a DUF206 domain.

171. The method of claim 170, wherein said LBD polypeptide comprises one or more of the following motifs:

| | |
|---|---|
| (i) Motif 1: MSCNGCRXLRKGCX, | (SEQ ID NO: 5) |
| (ii) Motif 2: QXXATXFXAKFXGR, | (SEQ ID NO: 6) |
| and | |
| (iii) Motif 3: FXSLLXEAXG | (SEQ ID NO: 7) |

172. The method of claim 170, wherein said modulated expression is effected by introducing and expressing in a plant a nucleic acid encoding a LBD polypeptide.

173. The method of claim 172, wherein said nucleic acid encoding a LBD polypeptide encodes any one of the proteins listed in Table A1 or is a portion of such a nucleic acid, or a nucleic acid capable of hybridizing with such a nucleic acid.

174. The method of claim 172, wherein said nucleic acid sequence encodes an orthologue or paralogue of any of the proteins given in Table A1.

175. The method of claim 170, wherein said enhanced yield-related traits comprise increased yield.

176. The method of claim 175, wherein said increased yield comprises increased biomass and/or increased seed yield relative to control plants.

177. The method of claim 170, wherein said enhanced yield-related traits are obtained under non-stress conditions.

178. The method of claim 170, wherein said enhanced yield-related traits are obtained under conditions of nitrogen deficiency.

179. The method of claim 172, wherein said nucleic acid is operably linked to a constitutive promoter.

180. The method of claim 179, wherein said constitutive promoter is a GOS2 promoter, preferably the GOS2 promoter from rice.

181. The method of claim 172, wherein said nucleic acid encoding a LBD polypeptide is of plant origin.

182. The method of claim 181, wherein said plant is a dicotyledonous plant.

183. The method of claim 182, wherein said dicotyledonous plant is from the family Brassicaceae.

184. The method of claim 183, wherein said dicotyledonous plant is Arabidopsis thaliana.

185. An isolated nucleic acid molecule comprising:
(i) the nucleic acid sequence represented by SEQ ID NO: 69;
(ii) a nucleic acid sequence or fragment thereof that is complementary to the sequence of SEQ ID NO: 69;
(iii) a nucleic acid sequence encoding a LBD polypeptide having at least 60% sequence identity to the sequence of SEQ ID NO: 70; or
(iv) a nucleic acid sequence capable of hybridizing under stringent conditions to any one of the nucleic acid sequences given in (i), (ii) and (iii) above.

186. An isolated polypeptide comprising:
(i) the amino acid sequence represented by SEQ ID NO: 70;
(ii) an amino acid sequence having at least 60% sequence identity to the amino acid sequence of SEQ ID NO: 70; or
(iii) derivatives of any of the amino acid sequences given in (i) and (ii).

187. A plant or part thereof, including seeds, obtainable by the method of claim 172, wherein said plant or part thereof comprises a recombinant nucleic acid encoding an LBD polypeptide.

188. A construct comprising:
(i) a nucleic acid sequence encoding an LBD polypeptide;
(ii) one or more control sequences capable of driving expression of the nucleic acid sequence of (i); and optionally
(iii) a transcription termination sequence,
wherein the LBD polypeptide comprises:
(a) a DUF206 domain;
(b) one or more of the motifs as defined in claim 171;
(c) the amino acid sequence represented by SEQ ID NO: 70;
(d) an amino acid sequence having at least 60% sequence identity to the amino acid sequence of SEQ ID NO: 70; or
(e) derivatives of any of the amino acid sequences given in (c) and (d).

189. The construct of claim 188, wherein one of said control sequences is a constitutive promoter.

190. The construct of claim 189, wherein said constitutive promoter is a GOS2 promoter, preferably the GOS2 promoter from rice.

191. A method for enhancing yield-related traits in plants relative to control plants, comprising using the construct of claim 188 to modulate expression in a plant of a nucleic acid encoding an LBD polypeptide, wherein said LBD polypeptide comprises a DUF206 domain.

192. A method for the production of a transgenic plant having increased yield, particularly increased biomass and/or increased seed yield relative to control plants, comprising:
(i) introducing and expressing in a plant a nucleic acid encoding an LBD polypeptide; and
(ii) cultivating the plant cell under conditions promoting plant growth and development,
wherein the nucleic acid is of plant origin and the LBD polypeptide comprises:
(a) a DUF206 domain; or
(b) one or more of the motifs as defined in claim 171.

193. The plant of claim 187 having increased yield resulting from increased expression of the nucleic acid encoding an LBD polypeptide, or a transgenic plant cell derived from said plant, wherein the nucleic acid is of plant origin and the LBD polypeptide comprises:
(a) a DUF206 domain; or
(b) one or more of the following motifs:

| | |
|---|---|
| (1) Motif 1: MSCNGCRXLRKGCX, | (SEQ ID NO: 5) |
| (2) Motif 2: QXXATXFXAKFXGR, | (SEQ ID NO: 6) |
| and | |
| (3) Motif 3: FXSLLXEAXG. | (SEQ ID NO: 7) |

194. The plant of claim 187, wherein said increased yield is increased biomass and/or increased seed yield, relative to control plants.

195. The plant of claim 187, or a transgenic plant cell derived thereof, wherein said plant is a crop plant or a monocot or a cereal, such as rice, maize, wheat, barley, millet, rye, triticale, sorghum and oats.
